SEQUENCE LISTING (2 sequences)

SEQ ID NO: 1 - DNA, 21 bp, Severe acute respiratory syndrome-related coronavirus
  source 1..21: mol_type = other DNA
cgcacggttatgtggaccctg

SEQ ID NO: 2 - RNA, 51 nt, synthetic construct
  source 1..51: mol_type = other RNA
ttttttttttttttttttttttttttttgtcattctcctaagaagctatta

(19) United States
(12) Patent Application Publication
(10) Pub. No.: US 2024/0327334 A1
(43) Pub. Date: Oct. 3, 2024

(54) Covalent Inhibitors As Antiviral Agents

(71) Applicant: Enanta Pharmaceuticals, Inc., Watertown, MA (US)

(72) Inventors: Xuri GAO, Newtonville, MA (US); Bin WANG, Newton, MA (US); Hui CAO, Belmont, MA (US); Jiajun ZHANG, Cambridge, MA (US); Yuk Ming SIU, Watertown, MA (US); Jiang LONG, Wayland, MA (US); Wei LI, Lexington, MA (US); Matthew C. RHODES, Boston, MA (US); Xuechao XING, Wilmington, MA (US); Jianming YU, Plainsboro, NJ (US); Scott MITCHELL, Woburn, MA (US); Yat Sun OR, Waltham, MA (US)

(21) Appl. No.: 18/390,287

(22) Filed: Dec. 20, 2023

Related U.S. Application Data
(60) Provisional application No. 63/434,190, filed on Dec. 21, 2022.

(51) Int. Cl. C07C 233/56; A61K 31/166; A61K 31/277; A61K 31/337; A61K 31/365; A61K 31/381; A61K 31/397; A61K 31/40; A61K 31/403; A61K 31/415; A61K 31/4152; A61K 31/416; A61K 31/4192; A61K 31/42; A61K 31/421; A61K 31/426; A61K 31/427; A61K 31/4402; A61K 31/4439; A61K 31/47; A61K 31/4709; A61K 31/495; A61K 31/4965; A61K 31/505; A61K 31/506; A61K 31/535; A61K 31/662; A61P 31/14; C07C 243/30; C07C 255/46; C07C 255/66; C07D 205/04; C07D 207/16; C07D 209/52; C07D 209/56; C07D 213/40; C07D 215/12; C07D 231/08; C07D 231/12; C07D 231/56; C07D 239/26; C07D 241/12; C07D 249/06; C07D 263/32; C07D 263/34; C07D 265/02; C07D 277/28; C07D 295/185; C07D 305/08; C07D 307/58; C07D 333/20; C07D 333/24; C07D 401/04; C07D 401/12; C07D 403/12; C07D 417/12; C07D 417/14; C07F 9/40

(52) U.S. Cl. CPC C07C 233/56; A61K 31/166; A61K 31/277; A61K 31/337; A61K 31/365; A61K 31/381; A61K 31/397; A61K 31/40; A61K 31/403; A61K 31/415; A61K 31/4152; A61K 31/416; A61K 31/4192; A61K 31/42; A61K 31/421; A61K 31/426; A61K 31/427; A61K 31/4402; A61K 31/4439; A61K 31/47; A61K 31/4709; A61K 31/495; A61K 31/4965; A61K 31/505; A61K 31/506; A61K 31/535; A61K 31/662; A61P 31/14; C07C 243/30; C07C 255/46; C07C 255/66; C07D 205/04; C07D 207/16; C07D 209/52; C07D 209/56; C07D 213/40; C07D 215/12; C07D 231/08; C07D 231/12; C07D 231/56; C07D 239/26; C07D 241/12; C07D 249/06; C07D 263/32; C07D 263/34; C07D 265/02; C07D 277/28; C07D 295/185; C07D 305/08; C07D 307/58; C07D 333/20; C07D 333/24; C07D 401/04; C07D 401/12; C07D 403/12; C07D 417/12; C07D 417/14; C07F 9/4015

(57) ABSTRACT

The present invention discloses compounds of Formula (I), and pharmaceutically acceptable salts, thereof:

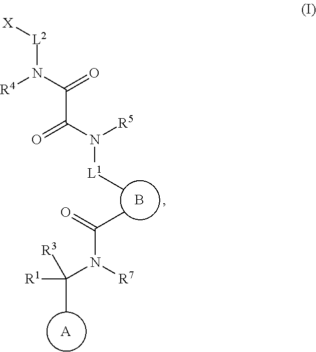

which inhibit coronavirus replication activity. The invention further relates to pharmaceutical compositions comprising a compound of Formula (I) or a pharmaceutically acceptable salt thereof, and methods of treating or preventing a coronavirus infection in a subject in need thereof, comprising administering to the subject a therapeutically effective amount of a compound of Formula (I) or a pharmaceutically acceptable salt thereof.

### RELATED APPLICATION

This application claims the benefit of U.S. Provisional Application No. 63/434,190, filed on Dec. 21, 2022. The entire teachings of the above application are incorporated herein by reference.

### REFERENCE TO AN ELECTRONIC SEQUENCE LISTING

The contents of the electronic Sequence Listing titled 40141390 US1 Seq List.xml (Size 2,966 bytes; and Date of Creation: Apr. 3, 2024) is herein incorporated by reference in its entirety.

### TECHNICAL FIELD

The invention relates to compounds and methods for treating or preventing a coronavirus infection. The invention further relates to pharmaceutical compositions comprising a compound of the invention.

### BACKGROUND OF THE INVENTION

Coronaviruses are enveloped, positive-sense, single-stranded RNA viruses. The genomic RNA of CoVs has a 5′-cap structure and 3′-poly-A tail and contains at least 6 open reading frames (ORFs). The first ORF (ORF 1a/b) directly translates two polyproteins: pp1a and pp1ab. These polyproteins are processed by two essential proteases 3C-Like protease (3CLpro), also known as the main protease (Mpro), and Papain-Like protease (PLpro) into 16 non-structural proteins. These non-structural proteins engage in the production of subgenomic RNAs that encode four structural proteins, namely envelope, membrane, spike, and nucleocapsid proteins, among other accessory proteins. As a result, it is understood that both 3CLpro and PLpro have critical roles in the coronavirus life cycle.

In addition, PLpro is involved in antagonizing the host's immune response upon viral infection. PLpro has deubiquitinating and deISGylating activities and removes ubiquitin and ISG15 modifications from host proteins, leading to suppression of the innate immune response and promotion of viral replication. The deubiquitinating and deISGylating activities of PLpro are indispensable in antagonizing the host's immune response. Recent studies showed that SARS-CoV-2 infection of human macrophages triggers the release of extracellular free ISG15 through the viral PLpro, leading to the subsequent secretion of proinflammatory cytokines and chemokines, which recapitulates the cytokine storm of COVID-19. This finding suggests that inhibiting the PLpro activity might alleviate the hyper-inflammation in COVID patients. Thus, targeting PLpro is expected to not only suppress viral replication but also restore antiviral immunity in the host.

There are two types of PLpros: PL1pro and PL2pro. They have distinct substrate specificities in different coronaviruses. The coronaviruses HCoV-220E, HCoV-NL63, HCoV-HKU1, and HCoV-OC43 encode both PL1pro and PL2pro. In contrast, SARS-CoV, MERS-CoV, and SARS-CoV-2 comprise only one functional PL2pro. The SARS-CoV-2 PLpro is part of nsp3, a 215-kDa multidomain viral protein. It specifically recognizes a consensus cleavage motif, LXGG↓(N/L/X), which is present in between nsp1/2, nsp2/3, and nsp3/4 at the viral polyprotein as well as the C-terminal sequences of ubiquitin and ISG15 with an isopeptide bond.

PLpro is a cysteine protease, containing four domains: the thumb, palm, zinc-finger domain, and an N-terminal ubiquitin-like domain. The catalytic triad consists of Cys, His and Asp, which are located at the interface of the palm and thumb domains. The zinc-finger motif comprises four cysteines coordinating with a zinc ion and is vital for the structural integrity and protease activity of PLpro. The flexible BL2 loop undergoes conformational changes from open to closed upon substrate binding. This site is also the drug binding site for GRL-0617 and its analogs which have been reported to inhibit PLpro activity (refer to WO2021/189046, WO2022/070048, WO2022/072975, WO2022/169891, WO2022/189810, WO2022/192665, WO 2023/028286, WO 2023/064493, and WO 2023/223055). However, no PLpro inhibitor has been advanced to the clinic.

Although Paxlovid (3CLpro inhibitor) and Lagevrio (RdRp inhibitor) have been approved as the first-generation oral antiviral therapies via EUA, more effective oral therapies with different MOAs for coronavirus infections are needed because the new therapies could overcome the potential drug resistance of current therapies. This invention describes the methods to prepare and methods for use of compounds that are believed to inhibit the coronavirus lifecycle. Compounds of this type might be used to treat coronavirus infections and decrease the occurrence of disease complications such as organ failure or death.

There is a need in the art for novel therapeutic agents that treat, ameliorate or prevent coronavirus infection. Administration of these therapeutic agents to a coronavirus infected patient, either as monotherapy or in combination with other coronavirus treatments or ancillary treatments, will lead to significantly improved prognosis, diminished progression of the disease, and enhanced seroconversion rates.

### SUMMARY OF THE INVENTION

The present invention relates to novel antiviral compounds, pharmaceutical compositions comprising such compounds, and methods for treating or preventing a viral (particularly coronavirus) infection in a subject in need of such therapy with said compounds. In addition, the present invention provides processes for the preparation of said compounds.

Compounds of the present invention inhibit the coronavirus Papain-Like protease (PLpro), thus interfering with the life cycle of the coronavirus and restoring host antiviral immunity.

The present invention provides compounds represented by Formula (I), and pharmaceutically acceptable salts, esters and prodrugs thereof,

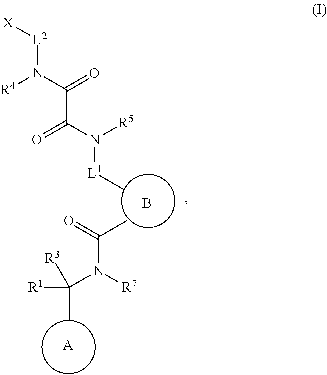

wherein:

A is selected from the group consisting of optionally substituted aryl, optionally substituted heteroaryl, optionally substituted —C₃-C₈ cycloalkyl, and optionally substituted 3- to 8-membered heterocycloalkyl;

B is selected from the group consisting of optionally substituted aryl, optionally substituted heteroaryl, optionally substituted —C₃-C₈ cycloalkyl, and optionally substituted 3- to 8-membered heterocycloalkyl;

R¹ and R³ are each independently selected from the group consisting of hydrogen, optionally substituted —C₁-C₄ alkyl, optionally substituted —C₂-C₄ alkenyl, optionally substituted —C₂-C₄ alkynyl, and optionally substituted —C₃-C₆ cycloalkyl;

alternatively, R¹ and R³ are taken together with the carbon atom to which they are attached to form an optionally substituted 3- to 8-membered carbocyclic or 3- to 8-membered heterocyclic ring;

R⁴, R⁵, and R⁷ are each independently selected from the group consisting of hydrogen, optionally substituted —C₁-C₆ alkyl, optionally substituted —C₂-C₆ alkenyl, optionally substituted —C₂-C₆ alkynyl, -optionally substituted —C₃-C₈ cycloalkyl, -optionally substituted 3- to 8-membered heterocycloalkyl, -optionally substituted aryl, and -optionally substituted heteroaryl;

L¹ and L² are independently selected from the group consisting of —(CR²¹R²³)_{q}, —CR²¹═CR²²—, —C≡C—, —(CR²¹R²³)_{q}—O—, —(CR²¹R²³)_{q}—(CR²¹R²³)_{q}—NR¹²—,

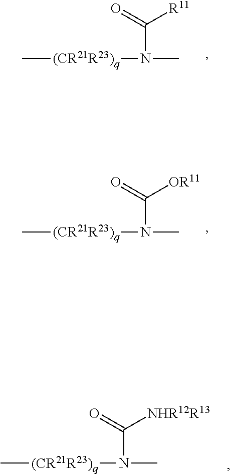

—(CR²¹R²³)_{q}—NR¹²C(O)—, —(CR²¹R²³)_{q}NR¹²C(O)O—, —(CR²¹R²³)_{q}NR¹²C(O)NR¹³, —(CR²¹R²³)_{q}—C(O)N(R¹²)—, —(CR²¹R²³)_{q}— N(R¹²)C(O)—, —(CR²¹R²³)_{q}—C(O)—, —(CR²¹R²³)_{q}—OC(O)—, —(CR²¹R²³)_{q}—S(O)₂—, —(CR²¹R²³)_{q}—S(O)—, —(CR²¹R²³)_{q}—S(O)(NR¹²)—, —(CR²¹R²³)_{q}—(NR¹²)S(O)—, —(CR²¹R²³)_{q}—S(O)₂NR¹²—, —(CR²¹R²³)_{q}—NR¹²S(O)₂—, optionally substituted —C₃-C₁₂ cycloalkyl, optionally substituted —C₃-C₁₂ cycloalkenyl, optionally substituted 3- to 12-membered heterocycloalkyl, optionally substituted aryl, optionally substituted arylalkyl, optionally substituted heteroaryl, and optionally substituted heteroarylalkyl;

L¹ connects to B through a carbon, nitrogen, sulfur, or oxygen atom;

q is 0, 1, 2, 3 or 4;

each R¹¹ is independently selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;

R¹² and R¹³ at each occurrence are independently selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; alternatively R¹² and R¹³ are taken together with the nitrogen atom to which they are attached to form an optionally substituted 3- to 8-membered heterocyclic ring;

R²¹ and R²² at each occurrence are independently selected from the group consisting of hydrogen, halogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; in certain embodiments, R^{2′} and R²² are both hydrogen;

R²³ at each occurrence is independently selected from the group consisting of hydrogen, halogen, —OR¹¹, —OC(O)R¹¹, —OC(O)OR¹¹, —OC(O)NR¹²R¹³, —NR¹²R¹³, —NR¹²C(O)R¹¹, —NR¹²C(O)OR¹³, —NR¹²C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; in certain embodiments, R²³ is hydrogen;

alternatively, R⁴ and L² or a substituent thereof are taken together with the intervening atoms to form an optionally substituted 3- to 8-membered heterocyclic ring;

X is selected from the group consisting of hydrogen, halogen, —CN, —C(O)R²⁵, —CH(OH)SO₃R²⁶, —C(O)NR²⁷R²⁸, —C(O)OR²⁷, —C(O)C(O)OR²⁷, —C(O)C(O)NR²⁷R²⁸, —C(O)(CR²¹R²¹)C(O)OR²⁷, —C(O)(CR²¹R²³)C(O)NR²⁷R²⁸, —C(O)S(O)₂NR²⁷R²⁸, —C(O)S(O)NR²⁷R²⁸,

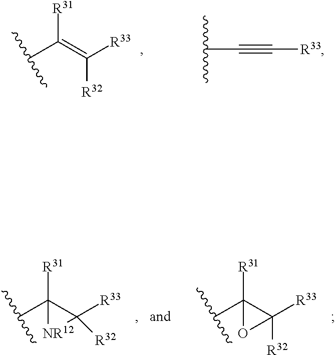

R²⁵ is hydrogen, halogen, hydroxy, or optionally substituted —C₁-C₈ alkyl; preferably R²⁵ is —CH₂OR¹¹, —CH₂F, or —CH₂Cl; wherein R¹¹ is as previously defined and is preferably hydrogen;

R²⁶ is hydrogen or Na⁺;

R²⁷ and R²⁸ at each occurrence are each independently selected from the group consisting of hydrogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;

alternatively, R²⁷ and R²⁸ are taken together with the nitrogen atom to which they are attached to form an optionally substituted 3- to 8-membered heterocyclic ring;

R³¹, R³² and R³³ are each independently selected from the group consisting of hydrogen, halogen, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted heteroaryl, —CN, —NO₂, —NO, —C(O)R²⁵, —CH(OH)SO₃R²⁶; —C(O)NR²⁷R²⁸, —C(O)OR²⁷, —C(O)SR²⁷, —S(O)₂R²⁷, —S(O)₂OR²⁷, —S(O)R²⁷, —S(O)OR²⁷, —S(O)₂NR²⁷R²⁸, —S(O)NR²⁷R²⁸, —P(O)R²⁷R²⁸, —P(O)OR²⁷R²⁸, —P(O)OR²⁷OR²⁸, —P(O)NR¹²R²⁷R²⁸, —P(O)NR¹²R¹³NR²⁷R²⁸, —P(O)NR¹²R¹³OR²⁸, —C(O)C(O)NR²⁷R²⁸, —C(O)C(O)OR²⁷, —C(O)S(O)₂NR²⁷R²⁸, and —C(O)S(O)NR²⁷R²⁸;
alternatively, R³¹ and R³³ are taken together with the carbon atoms to which they are attached to form an optionally substituted C₄-C₈ cycloalkyl, optionally substituted C₄-C₈ cycloalkenyl or optionally substituted 4- to 8-membered heterocyclic ring;
alternatively, R³² and R³³ are taken together with the carbon atom to which they are attached to form an optionally substituted C₃-C₈ cycloalkyl or optionally substituted 3- to 8-membered heterocyclic ring;
alternatively, R³¹ and L² or a substituent thereof are taken together with the nitrogen, carbon, sulfur, or oxygen atom to which they are attached to form an optionally substituted 3- to 8-membered heterocyclic ring;
alternatively, R³² and L² or a substituent thereof are taken together with the intervening atoms to form an optionally substituted 4- to 8-membered heterocyclic ring.

### DETAILED DESCRIPTION OF THE INVENTION

In one embodiment of the present invention is a compound of Formula (I) as described above, or a pharmaceutically acceptable salt thereof.

In certain embodiments of the compounds of Formula (I), R¹ is methyl or —CD₃.

In certain embodiments of the compounds of Formula (I), R³ is hydrogen.

In certain embodiments of the compounds of Formula (I), R¹ is methyl or —CD₃, and R³ is hydrogen.

In certain embodiments of the compounds of Formula (I), R¹ and R³ are taken together with the carbon atom to which they are attached to form an optionally substituted cyclopropyl.

In certain embodiments of the compounds of Formula (I), R⁴ is hydrogen or optionally substituted methyl.

In certain embodiments of the compounds of Formula (I), R⁵ is hydrogen or optionally substituted methyl.

In certain embodiments of the compounds of Formula (I), R⁷ is hydrogen or optionally substituted methyl.

In certain embodiments of the compounds of Formula (I), R⁴ is hydrogen, R⁵ is hydrogen, and R⁷ is hydrogen.

In certain embodiments of the compounds of Formula (I), A is optionally substituted aryl or optionally substituted heteroaryl.

In certain embodiments of the compounds of Formula (I), A is optionally substituted bicyclic aryl or optionally substituted bicyclic heteroaryl. In certain embodiments A is optionally substituted naphthyl, such as optionally substituted 1-naphthyl, optionally substituted 5,6-fused bicyclic heteroaryl, optionally substituted 6,6-fused bicyclic heteroaryl, optionally substituted benzo-fused 5-membered heteroaryl, or optionally substituted benzo-fused 6-membered heteroaryl.

In certain embodiments of the compounds of Formula (I), A is derived from one of the following by removal of one hydrogen atom and is optionally substituted:

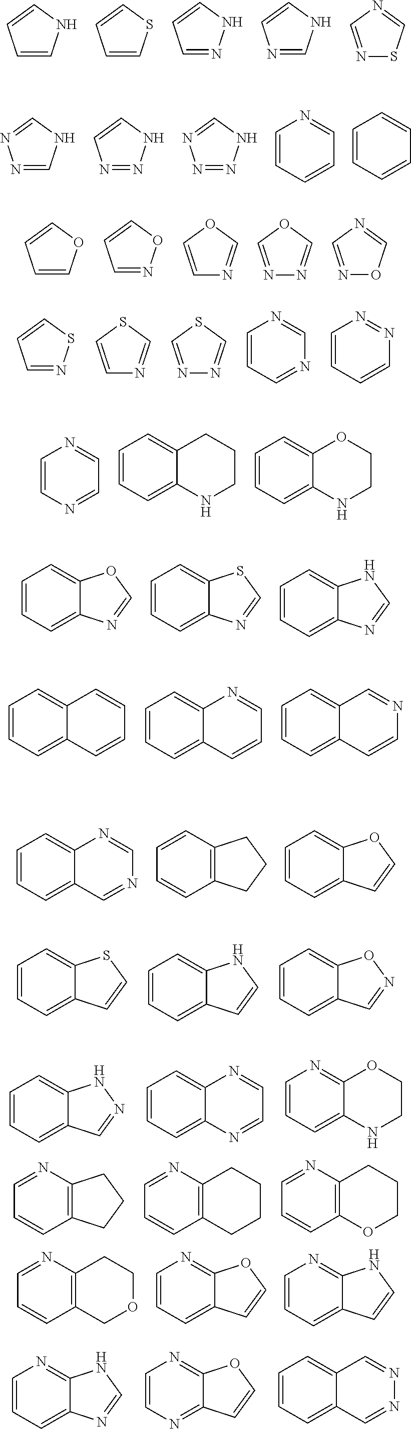

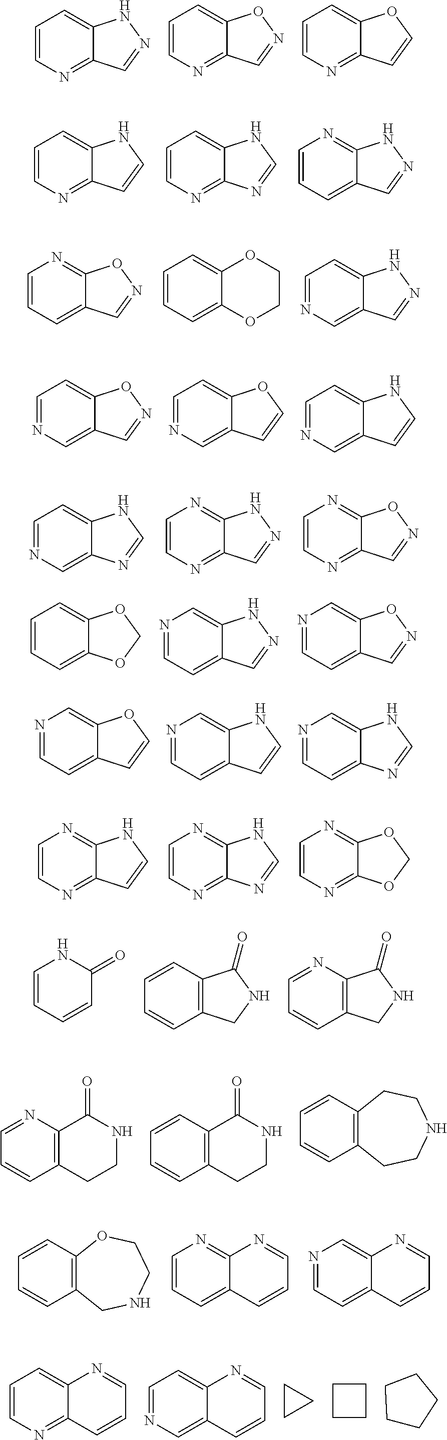

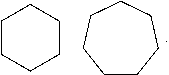

In certain embodiments of the compounds of Formula (I), A is optionally substituted phenyl, optionally substituted biphenyl, or optionally substituted naphthyl, such as optionally substituted 1-naphthyl or 2-naphthyl.

In certain embodiments of the compounds of Formula (I), B is optionally substituted aryl or optionally substituted heteroaryl.

In certain embodiments of the compounds of Formula (I), B is derived from one of the following by removal of two hydrogen atoms and is optionally substituted:

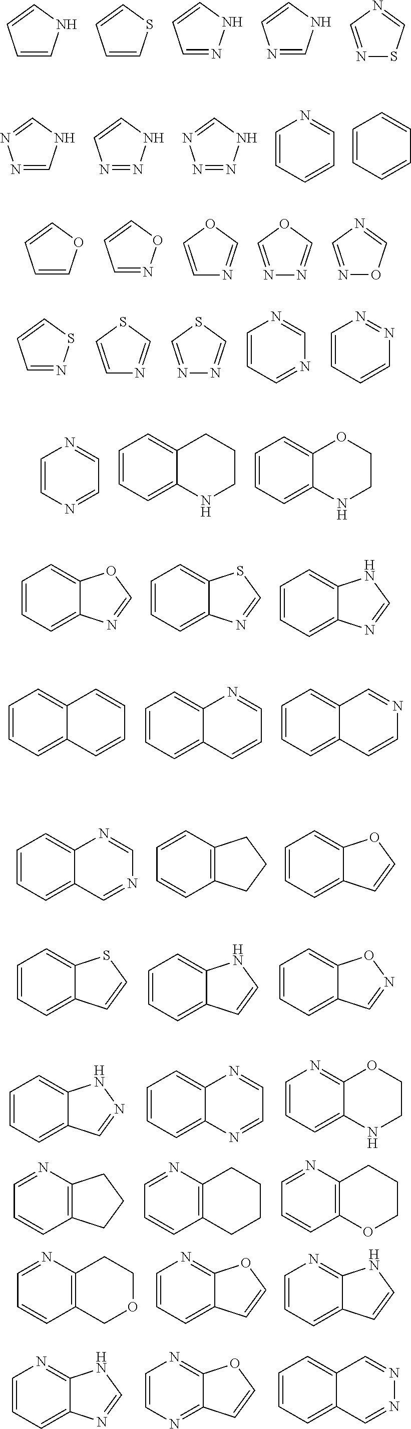

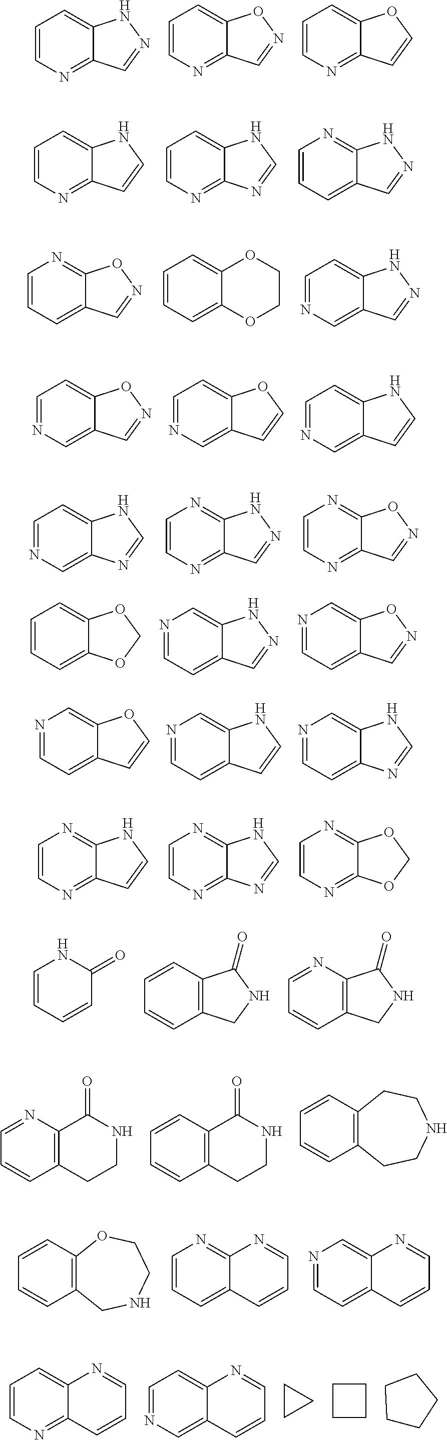

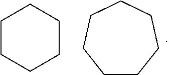

In certain embodiments of the compounds of Formula (I), B is optionally substituted phenyl, optionally substituted pyridyl, or optionally substituted cycloalkyl.

In certain embodiments, B is attached to L¹ and the amide group via two adjacent carbon atoms.

In certain embodiments of the compounds of Formula (I), L¹ is —CH₂—, —CH₂—CH₂—, —CH═CH—, —C(O)NR¹²—, —CH₂—NR¹²—, —CH₂—NR¹²C(O)—, or —O—CH₂—.

In certain embodiments of the compounds of Formula (I), L¹ is —CH₂—, or —CH₂—CH₂—.

In certain embodiments of the compounds of Formula (I), L² is-CH₂—, —CH₂—CH₂—, —CH═CH—, —C(O)NR¹²—, —O—CH₂—, —NR¹²—CH₂—, optionally substituted heteroaryl, or optionally substituted heterocycloalkyl. In one embodiment L² connects to X and the nitrogen atom to which it is attached in Formula (I) independently through a carbon, nitrogen, or oxygen atom.

In certain embodiments of the compounds of Formula (I), L² is —CH₂—, or —CH₂—CH₂—.

In certain embodiments of the compounds of Formula (I), X is —CN, —C(O)C(O)OR²⁷, —C(O)C(O)NR²⁷R²⁸, —C(O)CR²¹R²³C(O)OR²⁷, —C(O)CR²¹R²³C(O)NR²⁷R²⁸,

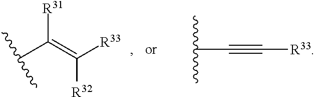

In certain embodiments of the compounds of Formula (I), X is

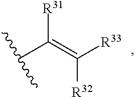

where R³¹, R³² and R³³ are as previously defined. Preferably no more than one of R³¹, R³² and R³³ is —CN, —NO₂, —NO, —C(O)R²⁵, —CH(OH)SO₃R²⁶; —C(O)NR²⁷R²⁸, —C(O)OR²⁷, —C(O)SR²⁷, —S(O)₂R²⁷, —S(O)₂OR²⁷, —S(O)R²⁷, —S(O)OR²⁷, —S(O)₂NR²⁷R²⁸, —S(O)NR²⁷R²⁸, —P(O)R²⁷R²⁸, —P(O)OR²⁷R²⁸, —P(O)OR²⁷OR²⁸, —P(O)NR¹²R²⁷R²⁸, —P(O)NR¹²R¹³NR²⁷R²⁸, —P(O)NR¹²R¹³OR²⁸, —C(O)C(O)NR²⁷R²⁸, —C(O)C(O)OR²⁷, —C(O)S(O)₂NR²⁷R²⁸, or —C(O)S(O)NR²⁷R²⁸. In certain embodiments R³¹ and R³³ or R³² and R³³ are not —CN, —NO₂, —NO, —C(O)R²⁵, —CH(OH)SO₃R²⁶; —C(O)NR²⁷R²⁸, —C(O)OR²⁷, —C(O)SR²⁷, —S(O)₂R²⁷, —S(O)₂OR²⁷, —S(O)R²⁷, —S(O)OR²⁷, —S(O)₂NR²⁷R²⁸, —S(O)NR²⁷R²⁸, —P(O)R²⁷R²⁸, —P(O)OR²⁷R²⁸, —P(O)OR²⁷OR²⁸, —P(O)NR¹²R²⁷R²⁸, —P(O)NR¹²R¹³NR²⁷R²⁸, —P(O)NR¹²R¹³OR²⁸, —C(O)C(O)NR²⁷R²⁸, —C(O)C(O)OR²⁷, —C(O)S(O)₂NR²⁷R²⁸, or —C(O)S(O)NR²⁷R²⁸. In certain embodiments, R³¹ and R³³ are hydrogen. In certain embodiments, R³² and R³³ are hydrogen.

In certain embodiments of the compounds of Formula (I), X is —CN,

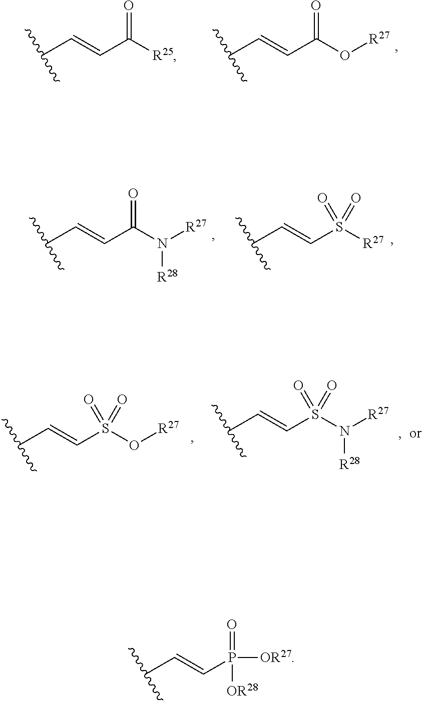

In certain embodiments, the compound of Formula (I) is represented by Formula (II):

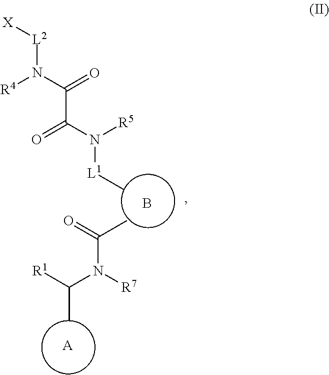

wherein A, B, R¹, R⁴, R⁵, R⁷, L¹, L², and X are as previously defined.

In certain embodiments, the compound of Formula (I) is represented by Formula (II-a):

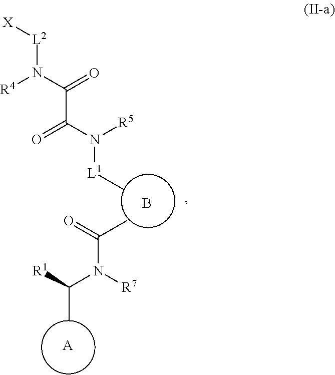

wherein A, B, R¹, R⁴, R⁵, R⁷, L¹, L², and X are as previously defined.

In certain embodiments, the compound of Formula (I) is represented by Formula (III):

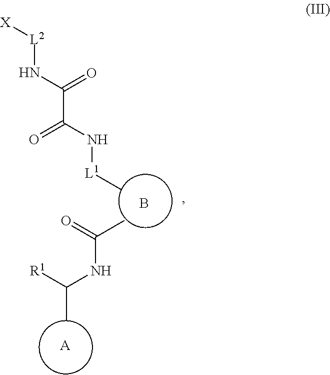

wherein A, B, R¹, L¹, L², and X are as previously defined.

In certain embodiments, the compound of Formula (I) is represented by Formula (IV):

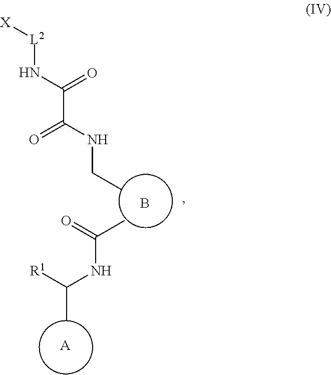

wherein A, B, R¹, L², and X are as previously defined.

In certain embodiments, the compound of Formula (I) is represented by Formula (V-1) or Formula (V-2):

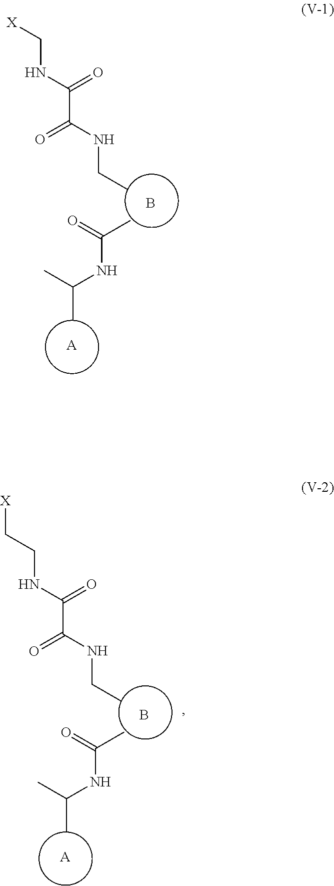

wherein A, B, and X are as previously defined.

In certain embodiments, the compound of Formula (I) is represented by Formula (V-1a) or Formula (V-2a):

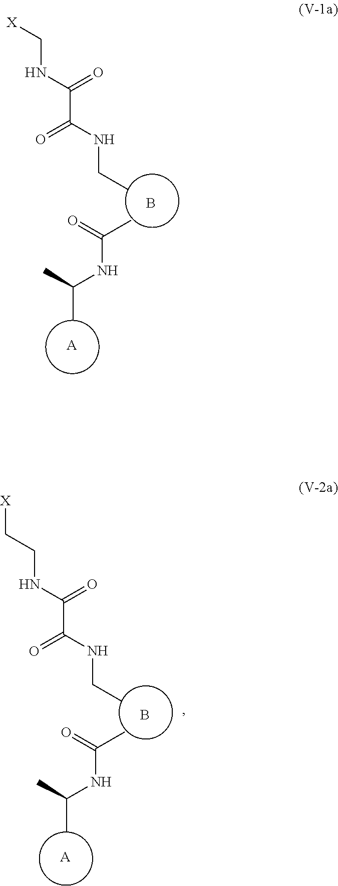

wherein A, B, and X are as previously defined.

In certain embodiments, the compound of Formula (I) is represented by one of Formulas (VI-1)˜(VI-3):

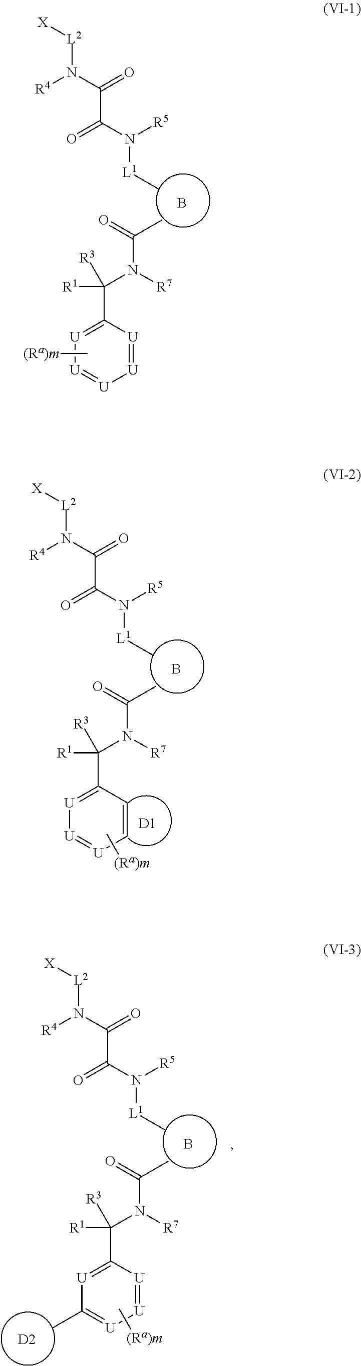

wherein one U is N or CH and the other U's are CH; D1 is selected from the group consisting of optionally substituted aryl, optionally substituted heteroaryl, optionally substituted —C₃-C₈ cycloalkenyl, and optionally substituted 3- to 8-membered unsaturated heterocycloalkyl; D2 is selected from the group consisting of optionally substituted aryl, optionally substituted heteroaryl, optionally substituted —C₃-C₈ cycloalkyl, optionally substituted —C₃-C₈ cycloalkenyl, and optionally substituted 3- to 8-membered heterocycloalkyl; R^{a} is selected from the group consisting of halogen, —OR¹¹, —OC(O)R¹¹, —C(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; and each m is independently 0, 1, 2, 3; or R¹¹, R¹², R¹³, B, R¹, R³, R⁴, R⁵, R⁷, L¹, L², and X are as previously defined. Preferably, D is phenyl or pyridyl, and m is 0 or 1.

In certain embodiments, the compound of Formula (I) is represented by one of Formulas (VI-1a)˜(VI-4a):

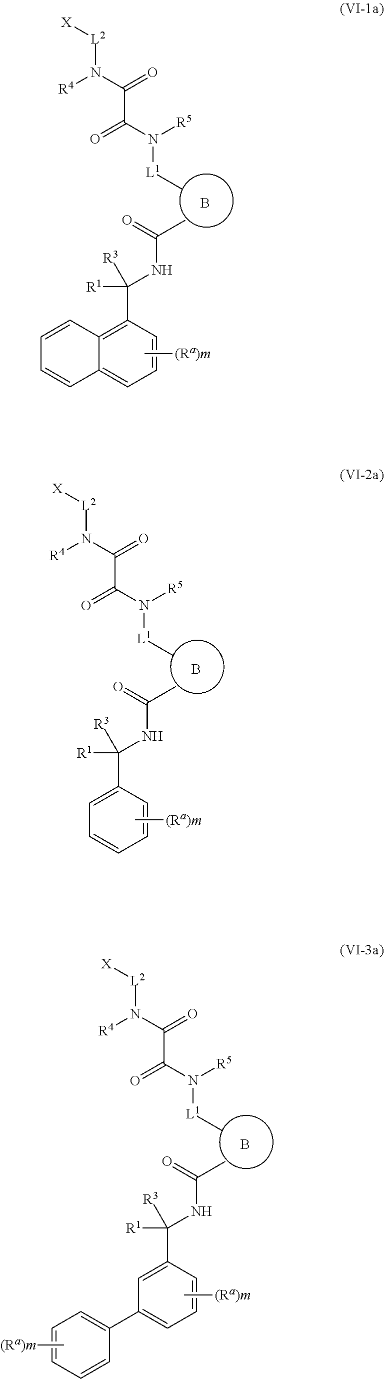

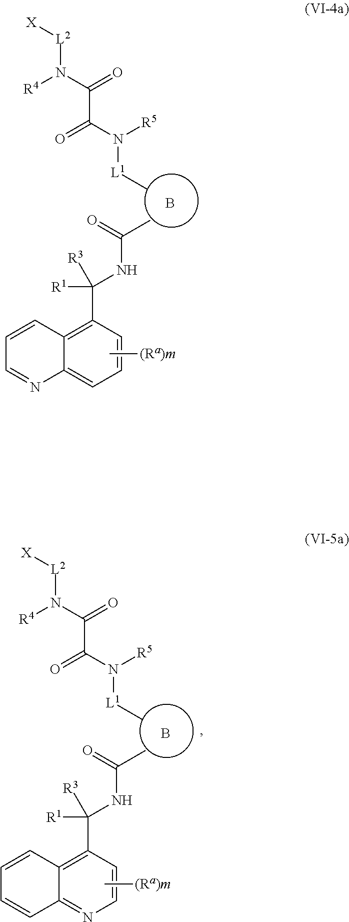

wherein U, R^{a}, m, B, R¹, R³, R⁴, R⁵, L¹, L², and X are as previously defined.

In certain embodiments, the compound of Formula (I) is represented by one of Formulas (VII-1)˜(VII-2):

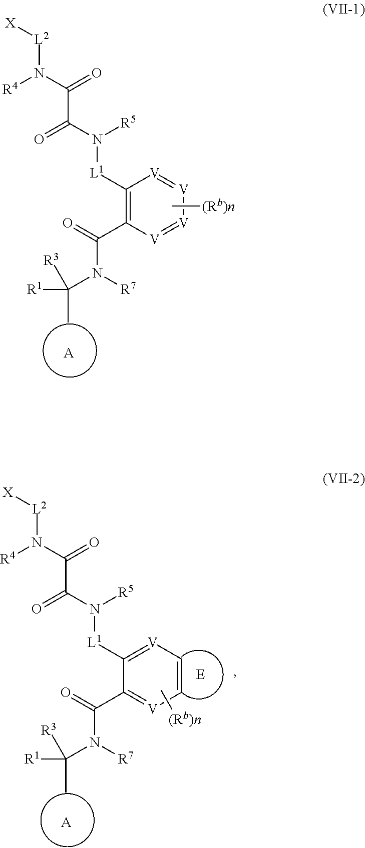

wherein one V is N or CH, the other Vs are CH; R^{b} selected from the group consisting of halogen, —OR¹¹, —OC(O)R¹¹, —C(O)OR¹¹, —OC(O)NR¹²R¹³, —NR¹²R¹³—NR¹²C(O)R¹¹, —NR¹²C(O)OR¹³, —NR¹²C(O)NR¹²R¹³, —C(O)NR¹²R¹³, —N₃, —CN, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; E is selected from the group consisting of optionally substituted aryl, optionally substituted heteroaryl, optionally substituted —C₃-C₈ cycloalkenyl, and optionally substituted 3- to 8-membered unsaturated heterocycloalkyl; each n is independently 0, 1, 2, 3; or A, R¹¹, R¹², R¹³, B, R¹, R³, R⁴, R⁵, R⁷, L¹, L², and X are as previously defined. Preferably, n is 0 or 1.

Alternatively, R⁵ and R^{b} are taken together with the intervening atoms to which they are attached form an optionally substituted 3- to 8-membered carbocyclic or 3- to 8-membered heterocyclic ring.

Alternatively, R^{b} and L¹ or a substituent thereof are taken together with the intervening atoms to form an optionally substituted 3- to 8-membered carbocyclic or 3- to 8-membered heterocyclic ring.

In certain embodiments, the compound of Formula (I) is represented by one of Formula (VII-1a)˜(VII-3a):

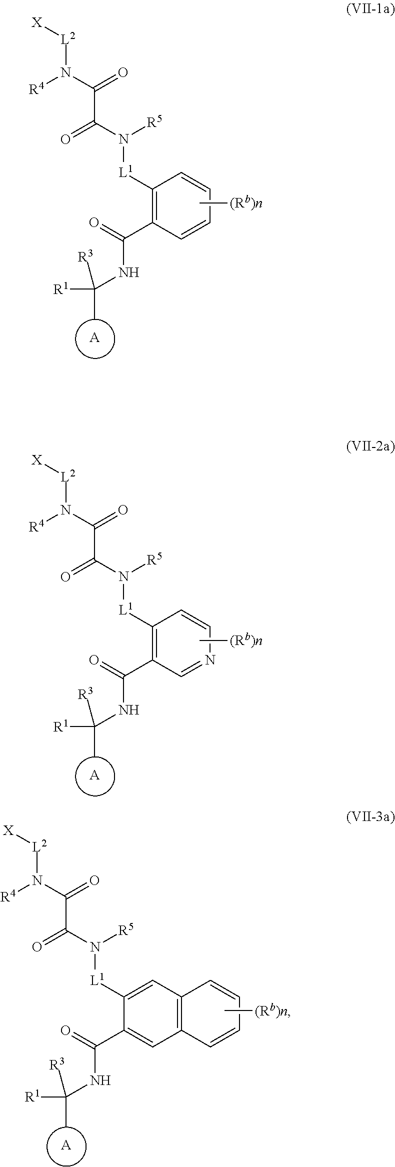

wherein R^{b}, n, A, R¹, R³, R⁴, R⁵, R⁷, L¹, L², and X are as previously defined.

In certain embodiments, the compound of Formula (I) is represented by Formula (VIII-1) or Formula (VIII-2):

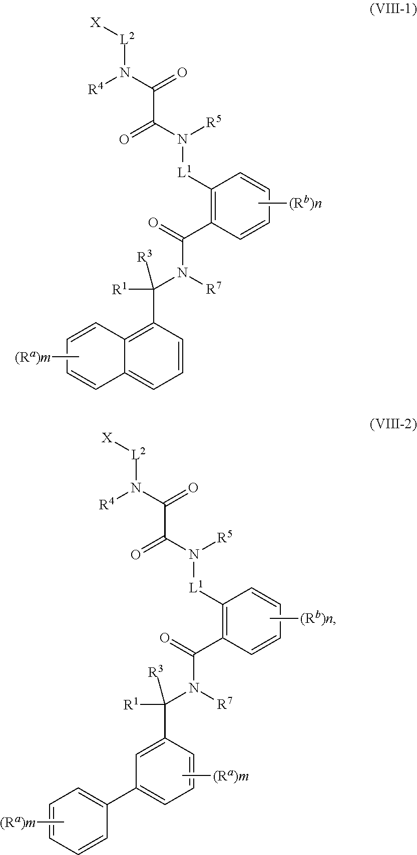

wherein m, n, R^{a}, R^{b}, R¹, R³, R⁴, R⁵, R⁷, L¹, L², and X are as previously defined.

In certain embodiments, the compound of Formula (I) is represented by Formula (IX-1) or Formula (IX-2):

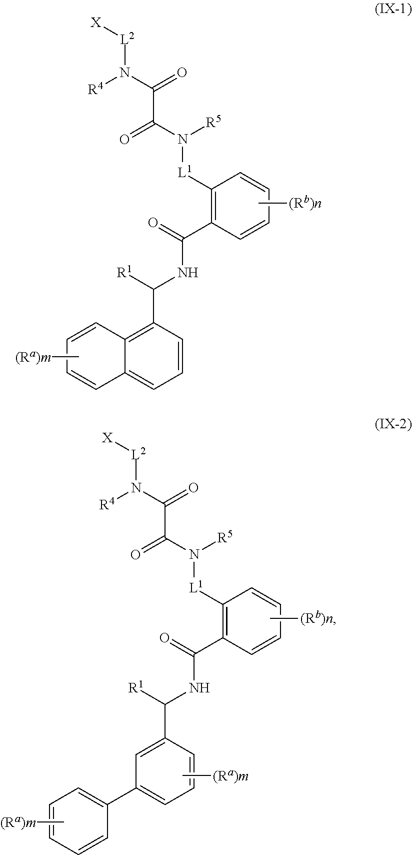

wherein m, n, R^{a}, R^{b}, R¹, R⁴, R⁵, L¹, L², and X are as previously defined.

In certain embodiments, the compound of Formula (I) is represented by Formula (X-1) or Formula (X-2):

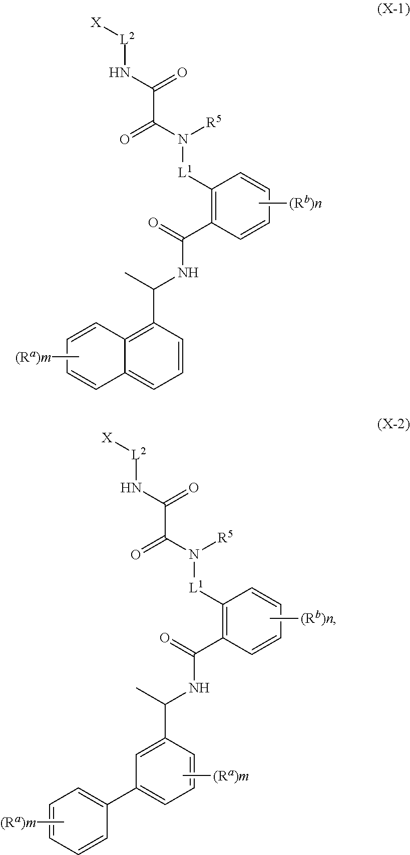

wherein m, n, R^{a}, R^{b}, L¹, L², and X are as previously defined.

In certain embodiments, the compound of Formula (I) is represented by one Formulae (XI-1)˜(XI-4):

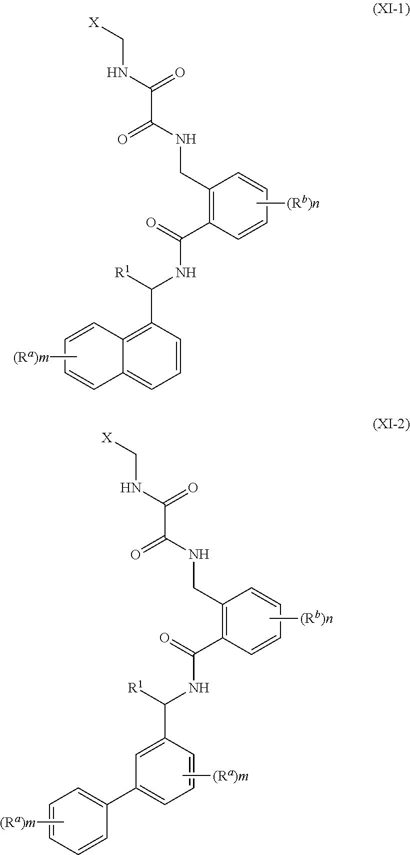

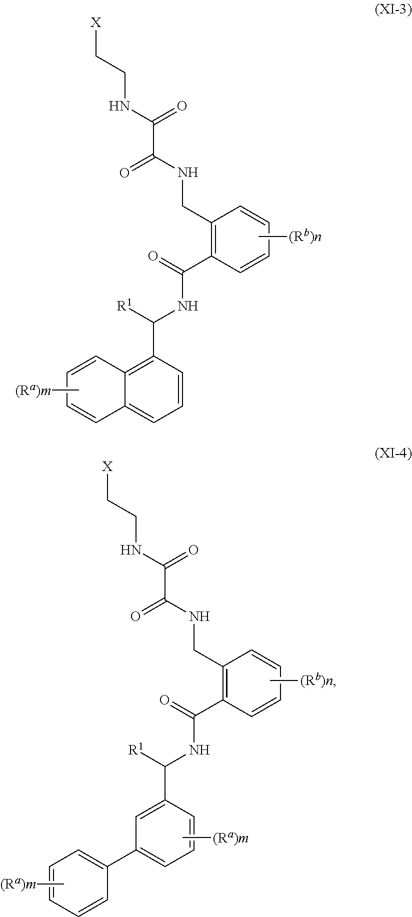

wherein m, n, R¹, R^{a}, R^{b}, and X are as previously defined. Preferably, X is —CN, —C(O)C(O)OR²⁷, —C(O)C(O)NR²⁷R²⁸, —C(O)CR²¹R²³C(O)OR²⁷, —C(O)CR²¹R²³C(O)NR²⁷R²⁸,

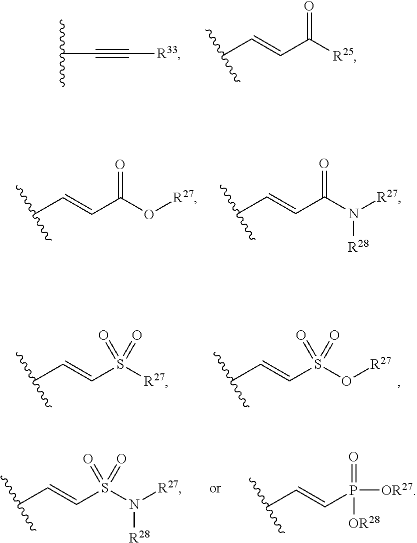

In certain embodiments, the compound of Formula (I) is represented by one of Formulas (XI-1a)˜(XI-4a):

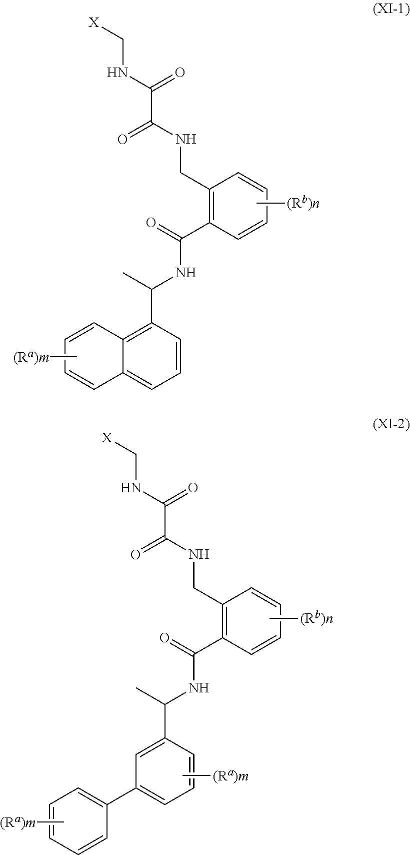

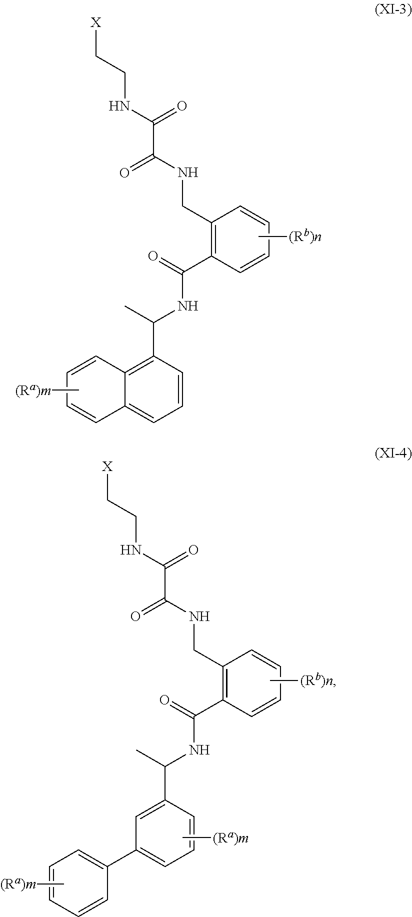

wherein m, n, R^{a}, R^{b}, and X are as previously defined. Preferably, X is —CN, —C(O)C(O)OR²⁷, —C(O)C(O)NR²⁷R²⁸, —C(O)CR²¹R²³C(O)OR²⁷, —C(O)CR²¹R²³C(O)NR²⁷R²⁸,

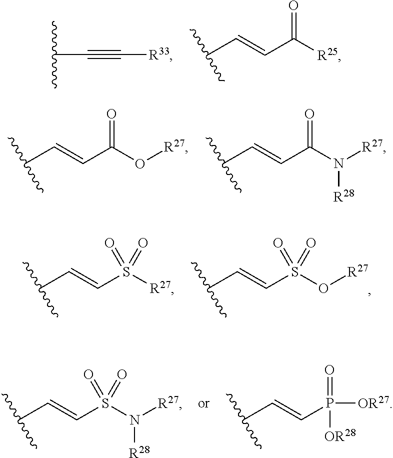

In certain embodiments, the compound of Formula (I) is represented by Formula (XII-1) or Formula (XII-2):

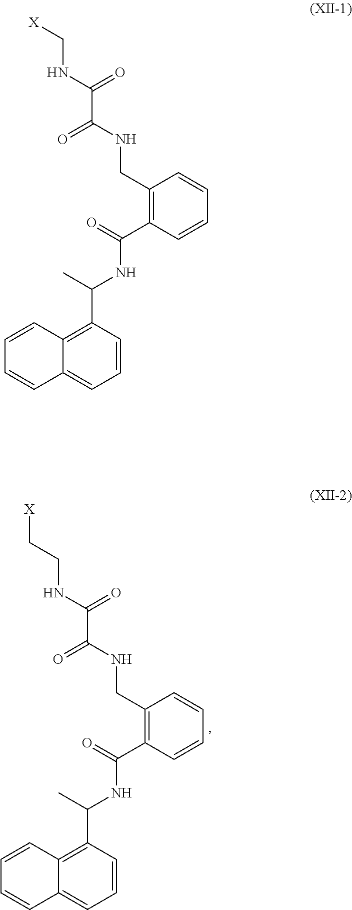

wherein X is as previously defined. Preferably, X is —CN, —C(O)C(O)OR²⁷, —C(O)C(O)NR²⁷R²¹, —C(O)CR²¹R²³C(O)OR²⁷, —C(O)CR²¹R²³C(O)NR²⁷R²⁸,

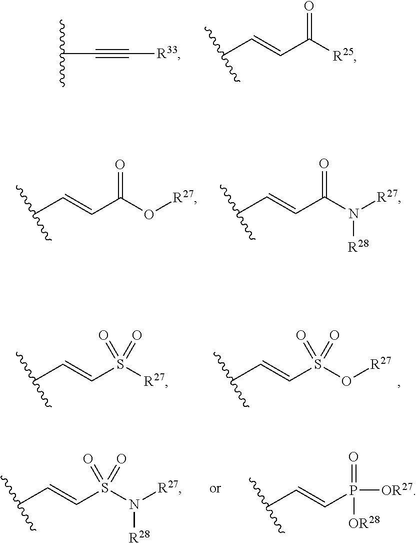

In certain embodiments, the compound of Formula (I) is represented by Formula (XIII):

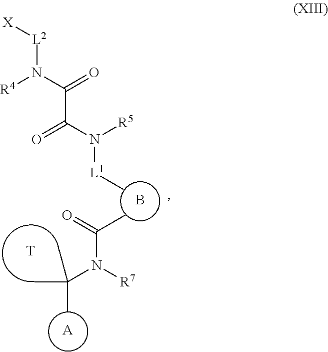

wherein T is selected from the group consisting of optionally substituted —C₃-C₈ cycloalkyl, optionally substituted 3- to 8-membered heterocycloalkyl, and optionally substituted —C₃-C₈ cycloalkenyl; A, B, R⁴, R⁵, R⁷, L¹, L², and X are as previously defined.

In certain embodiments, the compound of Formula (I) is represented by Formula (XIV):

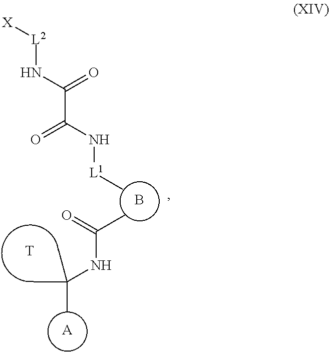

wherein T, A, B, L¹, L², and X are as previously defined.

In certain embodiments, the compound of Formula (I) is represented by Formula (XV-1) or Formula (XV-2):

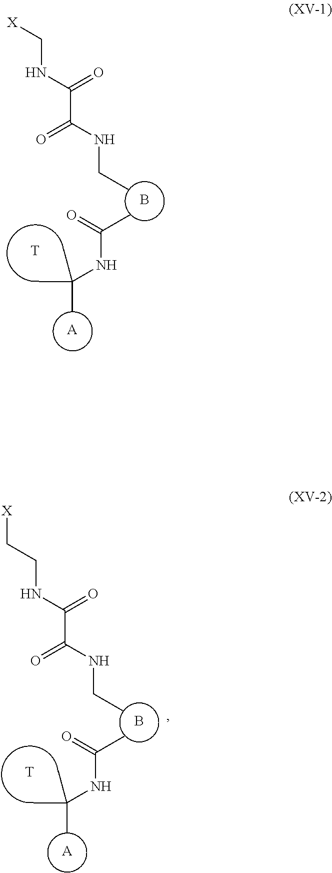

wherein T, A, B, and X are as previously defined.

In certain embodiments, the compound of Formula (I) is represented by one of Formulas (XVI-1)˜(XVI-3):

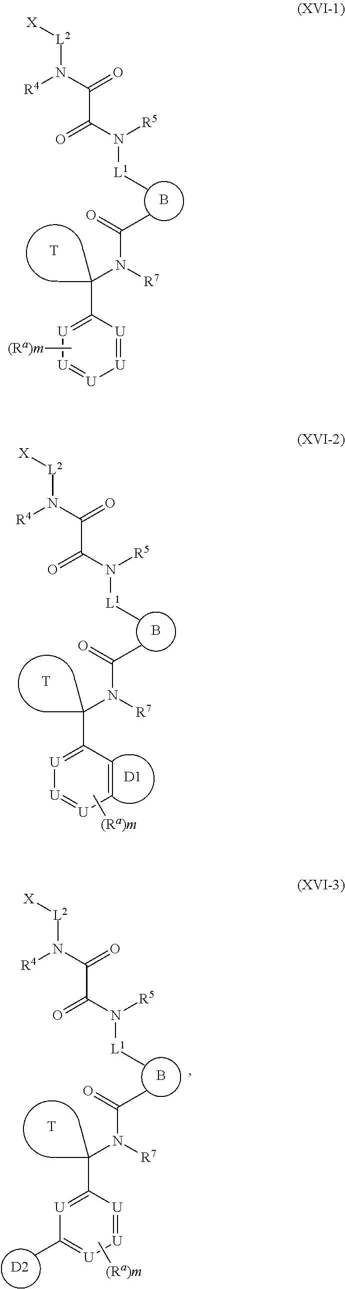

wherein R^{a}, m, U, D1, D2, T, B, R⁴, R⁵, R⁷, L¹, L², and X are as previously defined.

In certain embodiments, the compound of Formula (I) is represented by Formula (XVII-1) or Formula (XVII-2):

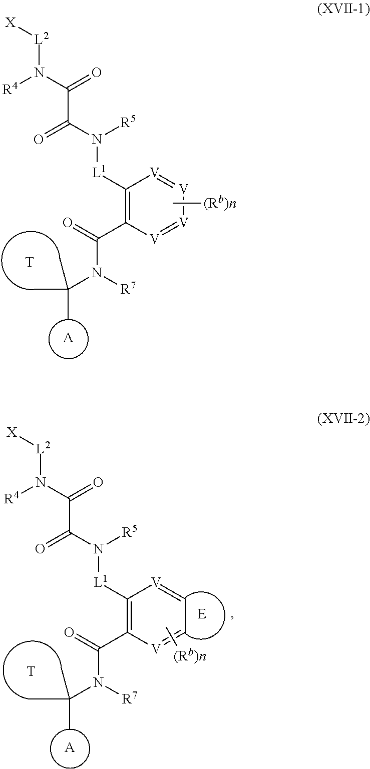

wherein R^{b}, n, E, V. T, A, R⁴, R⁵, R⁷, L¹, L², and X are as previously defined.

In certain embodiments, the compound of Formula (I) is represented by Formula (XVIII-1) or Formula (XVIII-2):

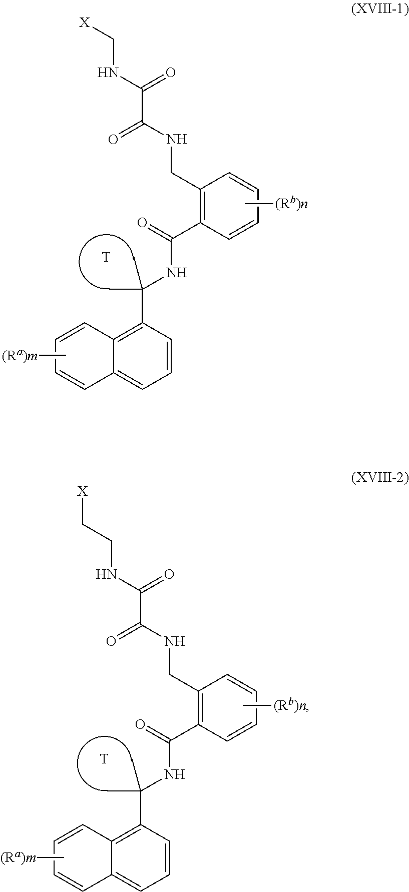

wherein R^{a}, m, R^{b}, n, T and X are as previously defined.

In certain embodiments, the compound of Formula (I) is represented by Formula (XIX-1) or Formula (XIX-2):

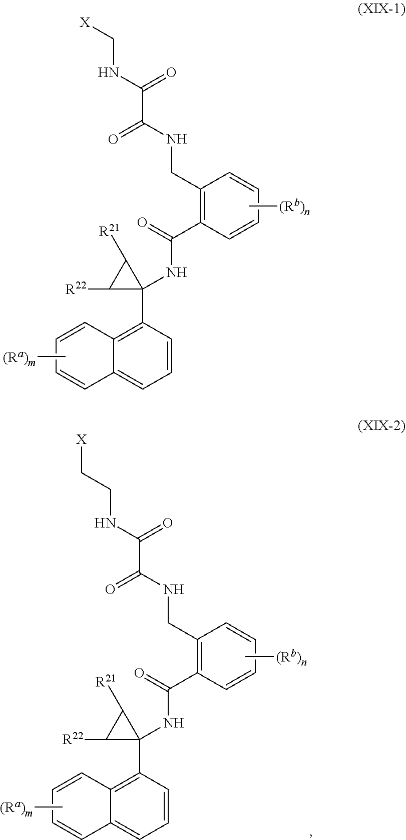

wherein R^{a}, m, R^{b}, n, R²¹, R²², and X are as previously defined. Preferably, R²¹ is hydrogen, R²² is hydrogen, and X is —CN, —C(O)C(O)OR²⁷, —C(O)C(O)NR²⁷R²⁸, —C(O)CR²¹R²³C(O)OR²⁷, —C(O)CR²¹R²³C(O)NR²⁷R²⁸,

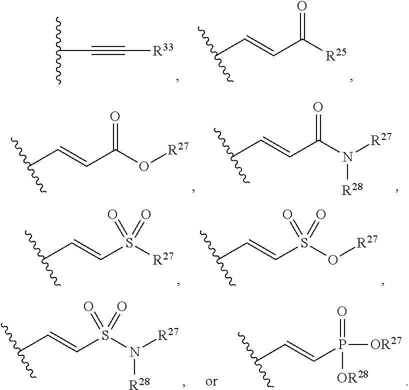

In certain embodiments, the compound of Formula (I) is represented by Formula (XX):

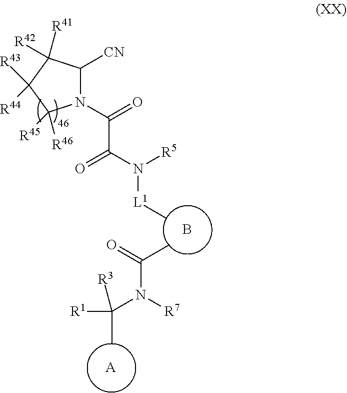

wherein R⁴¹, R⁴², R⁴³, R⁴⁴, R⁴⁵, and R⁴⁶ are independently selected from the group consisting of hydrogen, halogen, —OR²⁷, —SR²⁷, —NR²⁷R²⁸, —OC(O)NR²⁷R²⁸, optionally substituted —C₁-C₆ alkyl, optionally substituted —C₃-C₈ cycloalkyl, optionally substituted 3- to 8-membered hetereocycloalkyl, optionally substituted aryl, and optionally substituted heteroaryl; r is 0, 1, 2, or 3; A, B, R¹, R³, R⁵, R⁷, R²⁷, R²⁸, and L¹ are as previously defined;
alternatively, R⁴¹ and R⁴² are taken together to form 3- to 8-membered optionally substituted spiro ring;
alternatively, R⁴³ and R⁴⁴ are taken together to form 3- to 8-membered optionally substituted spiro ring;
alternatively, R⁴⁵ and R⁴⁶ are taken together to form 3- to 8-membered optionally substituted spiro ring;
alternatively, R⁴² and R⁴³ are taken together to form 3- to 8-membered optionally substituted fused ring;
alternatively, R⁴⁴ and R⁴⁵ are taken together to form 3- to 8-membered optionally substituted fused ring;
alternatively, R⁴² and R⁴⁵ are taken together to form 4- to 8-membered optionally substituted bridged ring;

In certain embodiments, the compound of Formula (I) is represented by Formula (XXI-1) or Formula (XXI-2):

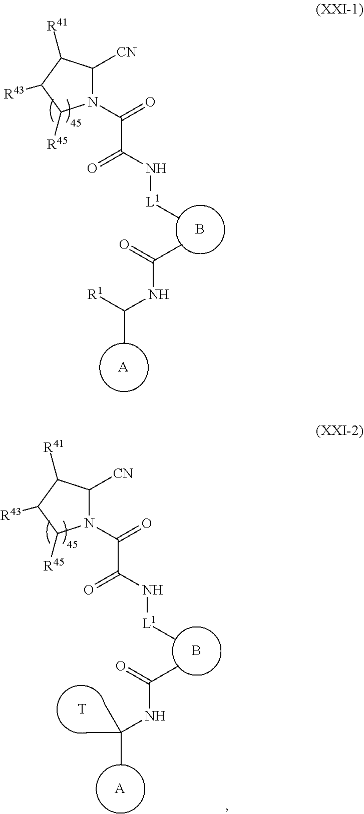

wherein A, B, T, R¹, R⁴¹, R⁴³, R⁴⁵, r, and L¹ are as previously defined.

In certain embodiments, the compound of Formula (I) is represented by one of Formulas (XXII-1)˜(XXII-4):

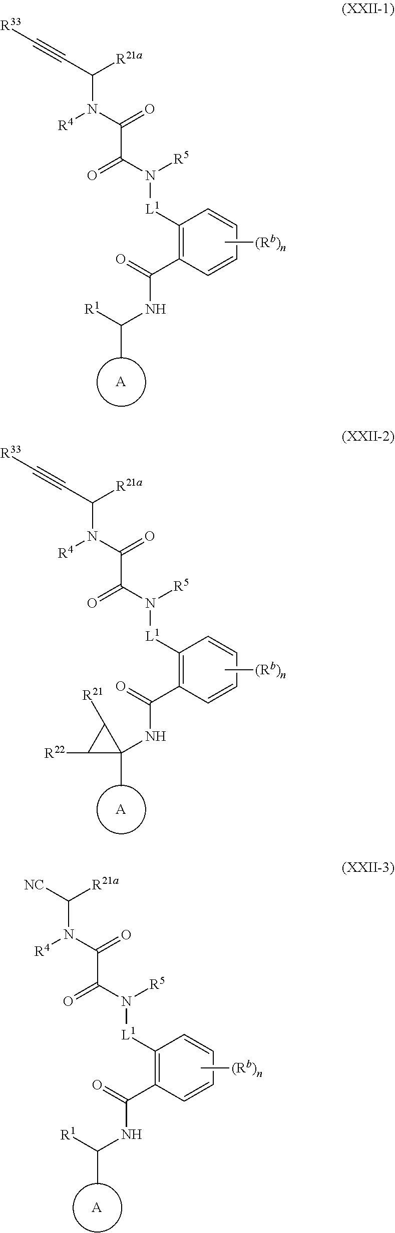

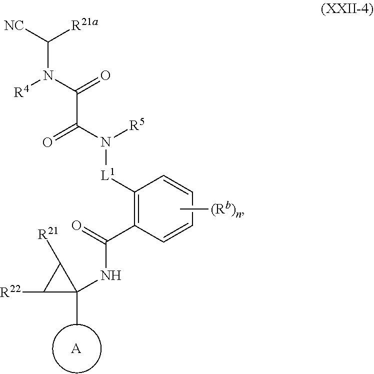

wherein A, R¹, R²¹, R²², R^{b}, n, L¹, R⁴, R⁵, and R³³ are as previously defined and R^{21a} is hydrogen or optionally substituted —C₁-C₆ alkyl.

In certain embodiments, the compound of Formula (I) is represented by one of Formulas (XXIII-1)˜(XXIII-4):

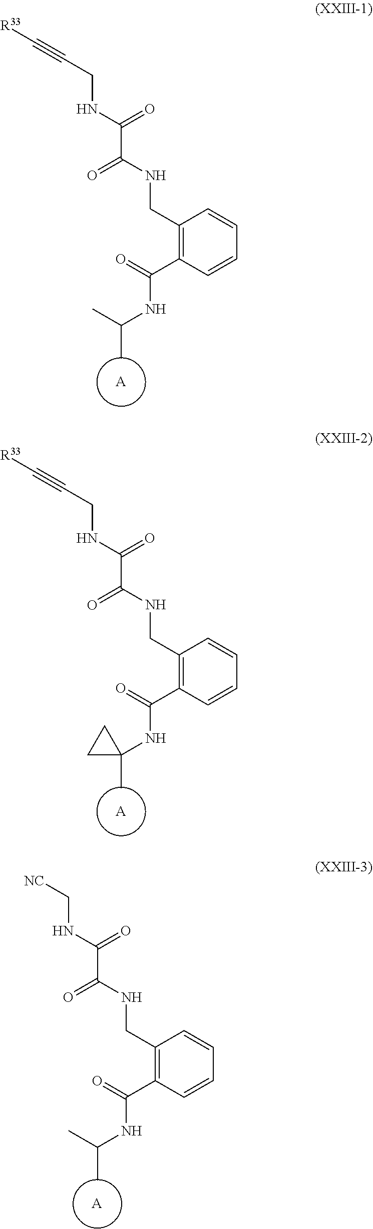

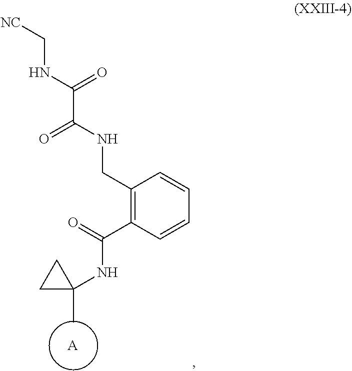

wherein A and R³³ are as previously defined. Preferably, R³³ is hydrogen, optionally substituted aryl, or optionally substituted heteroaryl.

In certain embodiments of the compounds of Formula (I) is represented by one of Formulas (XXII-1)˜(XXII-4) and Formulas (XXIII-1)˜(XXIII-4), wherein A is derived from one of the following by removal of one hydrogen atom and is optionally substituted:

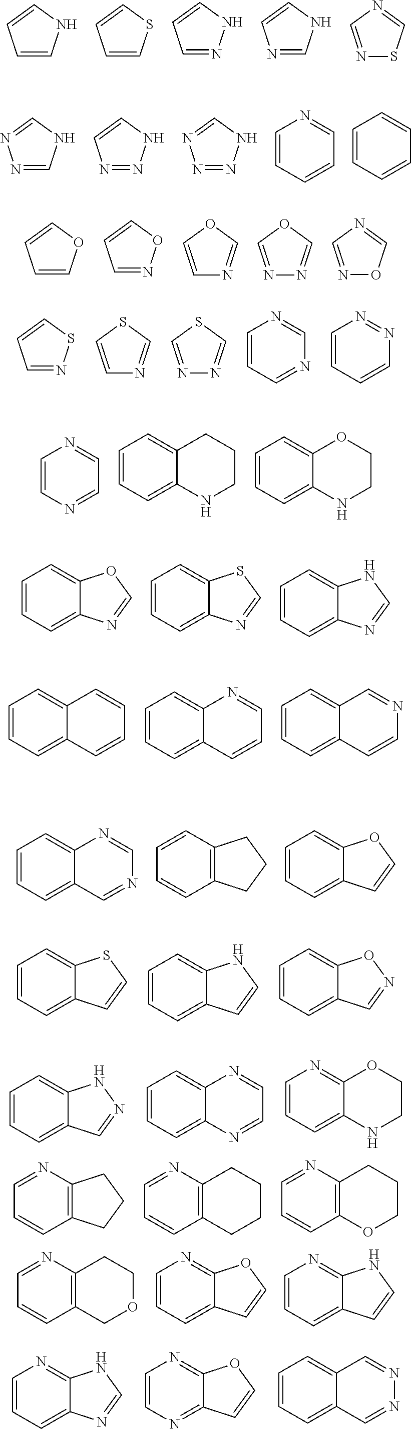

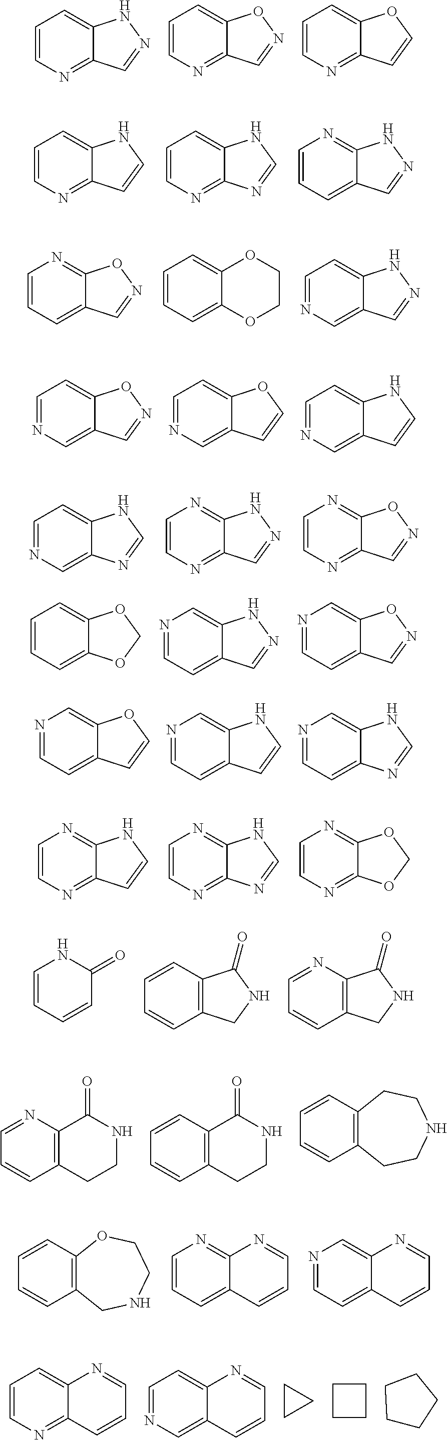

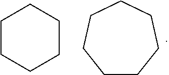

Preferably, A is selected from the groups below and is optionally substituted.

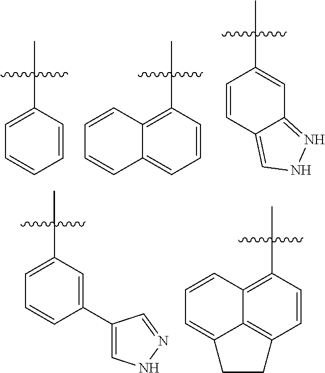

The present invention provides a pharmaceutical composition comprising a biologically active compound of the invention for the treatment of coronavirus in a mammal containing an amount of a coronavirus PLpro inhibitor that is effective in treating coronavirus and a pharmaceutically acceptable carrier or expedient.

The present invention provides a method of inhibiting the activity of a coronavirus PLpro, comprising contacting the coronavirus PLpro with an effective amount of a coronavirus PLpro inhibitor compound or agent.

The present invention also provides a method of targeting coronavirus inhibition as a means of treating indications caused by coronavirus related viral infections.

The present invention provides a method of treating or preventing a coronavirus infection in a subject in need thereof, comprising administering to the subject a therapeutically effective amount of a compound or a combination of compounds.

The present invention provides a method of treating or preventing a coronavirus infection in a subject in need thereof, further comprising administering to the subject an additional therapeutic agent selected from the group consisting of a coronavirus protease inhibitor, interferon, viral entry inhibitor, viral maturation inhibitor, inducer of cellular viral RNA sensor, therapeutic vaccine, and agents of distinct or unknown mechanism, and a combination thereof.

The present invention provides a method of reducing viral load in the subject to a greater extent compared to the administering of a compound selected from the group consisting of a coronavirus protease inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

The present invention provides a method resulting in a lower incidence of viral mutation and/or viral resistance than the treatment with a compound selected from the group consisting of a coronavirus protease inhibitor, interferon, viral entry inhibitor, viral maturation inhibitor, distinct capsid assembly modulator, inducer of cellular viral RNA sensor, therapeutic vaccine, antiviral compounds of distinct or unknown mechanism, and combination thereof.

In certain embodiments, the invention provides a method of treating or preventing a coronavirus infection in a subject, such as a human, in need thereof, comprising the step of administering to the subject a therapeutically effective amount of a compound of Formula (I) or a pharmaceutically acceptable salt thereof. The coronavirus can be an alpha, beta, gamma or delta coronavirus. In certain embodiments, the coronavirus is one which infects humans, such as coronavirus 229E, coronavirus NL63, coronavirus OC43, coronavirus HKU1, SARS-CoV-1, SARS-CoV-2, and MERS-CoV. In certain embodiments, the coronavirus is SARS-CoV-1, SARS-CoV-2, or MERS-CoV. Preferably the coronavirus is SARS-CoV-2.

Embodiments of the present invention provide administration of a compound to a healthy or virus-infected patient, either as a single agent or in combination with (1) another agent that is effective in treating or preventing coronavirus infections, (2) another agent that improves immune response and robustness, or (3) another agent that reduces inflammation and/or pain.

In a further aspect, this invention provides for a method of treating a respiratory disorder in a subject in need thereof, comprising administering to the subject a therapeutically effective amount of a compound of Formula (I) or a pharmaceutically acceptable salt thereof. Such respiratory disorders include, but are not limited to, an acute airway disease or a chronic airway disease. Examples of such respiratory disorders include acute asthma, lung disease secondary to environmental exposures, acute lung infection, and chronic lung infection.

It will be appreciated that the description of the present invention herein should be construed in congruity with the laws and principles of chemical bonding. In some instances, it may be necessary to remove a hydrogen atom in order to accommodate a substituent at any given location.

It will be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing coronavirus.

It should be understood that the compounds encompassed by the present invention are those that are suitably stable for use as pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy is C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One of skill in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, —CO₂—C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH— heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)— heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH— heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂—C₁-C₁₂-alkyl, —SO₂—C₂-C₈-alkenyl, —SO₂—C₂-C₈-alkynyl, —SO₂—C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S— heterocycloalkyl, or methylthio-methyl. In certain embodiments, the substituents are independently selected from halo, preferably C₁ and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, N Y, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, N J (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically acceptable carriers, adjuvants, or vehicles. In some cases, the pH of the formulation may be adjusted with pharmaceutically acceptable acids, bases, or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Combination and Alternation Therapy

The compounds of the present invention may be used in combination with one or more antiviral therapeutic agents or anti-inflammatory agents useful in the prevention or treatment of viral diseases or associated pathophysiology. Thus, the compounds of the present invention and their salts, solvates, or other pharmaceutically acceptable derivatives thereof, may be employed alone or in combination with other antiviral or anti-inflammatory therapeutic agents. The compounds herein and pharmaceutically acceptable salts thereof may be used in combination with one or more other agents which may be useful in the prevention or treatment of respiratory disease, inflammatory disease, autoimmune disease, for example; anti-histamines, corticosteroids, (e.g., fluticasone propionate, fluticasone furoate, beclomethasone dipropionate, budesonide, ciclesonide, mometasone furoate, triamcinolone, flunisolide), NSAIDs, Ieukotriene modulators (e.g., montelukast, zafirlukast.pranlukast), tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (Zyflo), DP1 antagonists, DP2 antagonists, PI3K delta inhibitors, ITK inhibitors, LP (Lysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-ethylpyridin-2-yl)methoxy)-1H-indol-2-yl)-2,2-dimethylpropanoate), bronchodilators (e.g., muscarinic antagonists, beta-2 agonists), methotrexate, and similar agents; monoclonal antibody therapy such as anti-IgE, anti-TNF, anti-IL-5, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents), suitable anti-infective agents including antibiotic agents, antifungal agents, antheimintic agents, antimalarial agents, antiprotozoal agents, antitubercuiosis agents, and antiviral agents, including those listed at https://www.drugs.com/drug-class/anti-infectives.html. In general, combination therapy is typically preferred over alternation therapy because it induces multiple simultaneous stresses on the virus.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The “additional therapeutic or prophylactic agents” include but are not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

Although the invention has been described with respect to various preferred embodiments, it is not intended to be limited thereto, but rather those skilled in the art will recognize that variations and modifications may be made therein which are within the spirit of the invention and the scope of the appended claims.

### Antiviral Activity

In certain embodiments, the present invention provides a method of treating or preventing a viral infection in a subject in need thereof, comprising administering to the subject a therapeutically effective amount of a compound of Formula (I) or a pharmaceutically acceptable salt thereof. The viral infection is preferably a coronavirus infection. In certain embodiments, the coronavirus is SARS-CoV-1, SARS-CoV-2, or MERS-CoV. Preferably the coronavirus is SARS-CoV-2.

A viral inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, viral infections are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). A therapeutically effective amount of the compound described above may range, for example, from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Abbreviations

Abbreviations which may be used in the descriptions of the scheme and the examples that follow are: Ac for acetyl; AcOH or HOAc for acetic acid; ACN or MeCN or CH₃CN for acetonitrile; BF₃·OEt₂ for boron trifluoride diethyl etherate; Boc₂O for di-tert-butyl-dicarbonate; Boc for t-butoxycarbonyl; Bz for benzoyl; Bn for benzyl; t-BuOK for potassium tert-butoxide; Brine for sodium chloride solution in water; CbzCl or Cbz-Cl for benzyl chloroformate; CDI for carbonyldiimidazole; DCM or CH₂Cl₂ for dichloromethane; CH₃ for methyl; (COCl)₂ for oxalyl chloride; Cl₂CHCN for dichloroacetonitrile; Cs₂CO₃ for cesium carbonate; CuCl for copper (I) chloride; CuI for copper (I) iodide; CuSO₄ for copper (II) sulfate; dba for dibenzylidene acetone; DBU for 1,8-diazabicyclo[5.4.0]-undec-7-ene; DCC for N,N′-dicyclohexylcarbodiimide; DCE for 1,2-dichloroethane; DIBAL-H for diisobutylaluminum hydride; DIPEA or (i-Pr)₂EtN for N,N-diisopropylethyl amine; DMP or Dess-Martin periodinane for 1,1,2-tris(acetyloxy)-1,2-dihydro-1,2-benziodoxol-3-(1H)-one; DMAP for 4-dimethylamino-pyridine; DME for 1,2-dimethoxyethane; DMF for N,N-dimethylformamide; DMSO for dimethyl sulfoxide; EDC for 1-ethyl-3-(3-dimethylaminopropyl)carbodiimide; EtOAc for ethyl acetate; EtOH for ethanol; Et₂O for diethyl ether; H₂ for hydrogen, HATU for O-(7-azabenzotriazol-2-yl)-N,N,N′,N′-tetramethyluronium Hexafluoro-phosphate; HCl for hydrogen chloride; K₂CO₃ for potassium carbonate; n-BuLi for n-butyl lithium; KHMDS for potassium bis(trimethylsilyl)amide; IBX for 2-iodoxybenzoic acid; In for indium; LDA for lithium diisopropylamide; Li for lithium; LiBH₄ for lithium borohydride; LiBr for lithium bromide; LiHMDS for lithium bis(trimethylsilyl)amide; LiOH for lithium hydroxide; LiTMP for lithium 2,2,6,6-tetramethyl-piperidinate; MeOH for methanol; Mg for magnesium; MOM for methoxymethyl; Ms for mesyl or —SO₂—CH₃; NaHMDS for sodium bis(trimethylsilyl)amide; NaCl for sodium chloride; NaBH₄ for sodium borohydride; NaBH(OAc)₃ for sodium triacetoxyborohydride; NaH for sodium hydride; NaHCO₃ for sodium bicarbonate or sodium hydrogen carbonate; Na₂CO₃ sodium carbonate; NaOH for sodium hydroxide; Na₂SO₄ for sodium sulfate; NaHSO₃ for sodium bisulfate or sodium hydrogen sulfite; Na₂S₂O₃ for sodium thiosulfate; NBS for N-bromosuccinimide; NH₃ for ammonia; NH₄OH for ammonium hydroxide; NH₂NH₂ for hydrazine; NH₄Cl for ammonium chloride; Ni for nickel; NMM for N-methylmorpholine; n-PrOH for 1-propanol; OH for hydroxyl; OsO₄ for osmium tetroxide; OTf for triflate; PPA for polyphophoric acid; PTSA or PTSOH for p-toluenesulfonic acid; PPTS for pyridinium p-toluenesulfonate; SiliaMetS DMT for the silica-bound equivalent of 2,4,6-trimercaptotriazine (trithiocyanuric acid, TMT); SO₃ for sulfur trioxide; TBAF for tetrabutylammonium fluoride; TEA or Et₃N or NEt₃ for triethylamine; TFA for trifluoroacetic acid; TFAA for trifluoroacetic anhydride; THE for tetrahydrofuran; T₃P for propylphosphonic anhydride; TPP or PPh₃ for triphenyl-phosphine; Tos or Ts for tosyl or —SO₂—C₆H₄CH₃; Ts₂O for tolylsulfonic anhydride or tosyl-anhydride; TsOH for p-tolylsulfonic acid; Pd for palladium; Pd/C for palladium on carbon; Ph for phenyl; Pd₂(dba)₃ for tris(diben-zylideneacetone) dipalladium (0); Pd(PPh₃)₄ for tetrakis(triphenylphosphine)-palladium (0); PdCl₂(PPh₃)₂ for trans-dichlorobis-(triphenylphosphine)palladium (II); PdCl₂(dppf) for [1,1′-bis(diphenylphosphino)ferrocene]palladium(II) dichloride; Pd(TFA)₂ for palladium(II) trifluoroacetate; for Pt for platinum; Rh for rhodium; rt for room temperature; Ru for ruthenium; TBS for tert-butyl dimethylsilyl; TMS for trimethylsilyl; or TMSCl for trimethylsilyl chloride; TMSOTf for trimethylsilyl trifluoromethanesulfonate; Zhan 1B cat. for dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II).

### Synthetic Methods

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

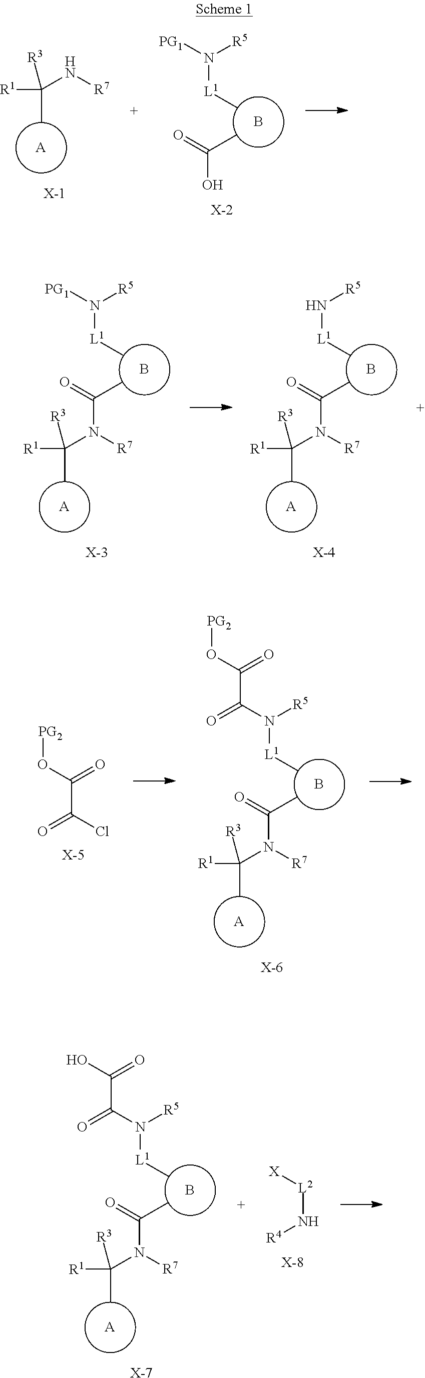

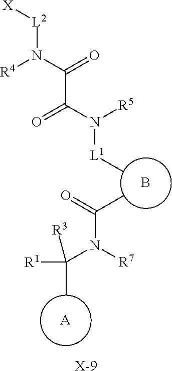

Scheme 1 illustrates a general method to prepare the compound (X-9) of formula I from the amine (X-1), wherein A, R¹, R³ and R⁷ are as previously defined. Amide coupling of X-1 with the carboxylic acid (X-2), wherein PG1 is a suitable amine protecting group, e.g. Boc, Cbz, Fmoc, etc., B, L¹, and R⁵ are as previously defined, provides the amide (X-3). Removal of PG₁ using the appropriate conditions, e.g. HCl, TFA, Pd/C with H₂, etc, gives the amine (X-4). Treatment of X-4 with the acid chloride (X-5), wherein PG₂ is a suitable ester protecting group, e.g. methyl, ether, Bn, etc., afford the amide (X-6), whose PG₂ is deprotected by using the appropriate conditions, e.g. LiOH, Pd/C with H₂, etc, to give the acid (X-7). Amide coupling of X-7 with the amine (X-8), wherein L², R⁴ and X are as previously defined, generated the final product (X-9).

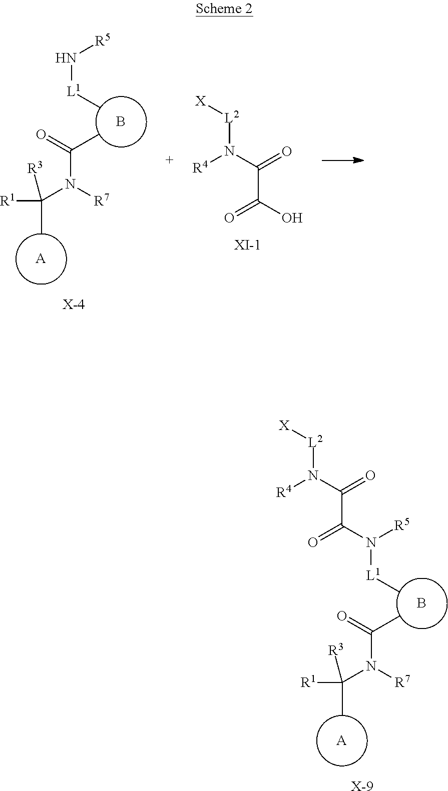

Scheme 2 illustrates an alternative method to prepare the compound (X-9) of formula I from the common intermediate (X-4), wherein A, B, R¹, R³, R⁵, R⁷ and L¹ are as previously defined. Amide coupling of the amine (X-4) with the carboxylic acid (XI-1), wherein R⁴, L², and X are as previously defined, affords the final product X-9.

### EXAMPLES

The compounds and processes of the present invention will be better understood in connection with the following examples, which are intended as an illustration only and not limiting of the scope of the invention. Starting materials were either available from a commercial vendor or produced by methods well known to those skilled in the art.

While this invention has been particularly shown and described with references to preferred embodiments thereof, it will be understood by those skilled in the art that various changes in form and details may be made therein without departing from the scope of the invention encompassed by the appended claims.

### Example 1

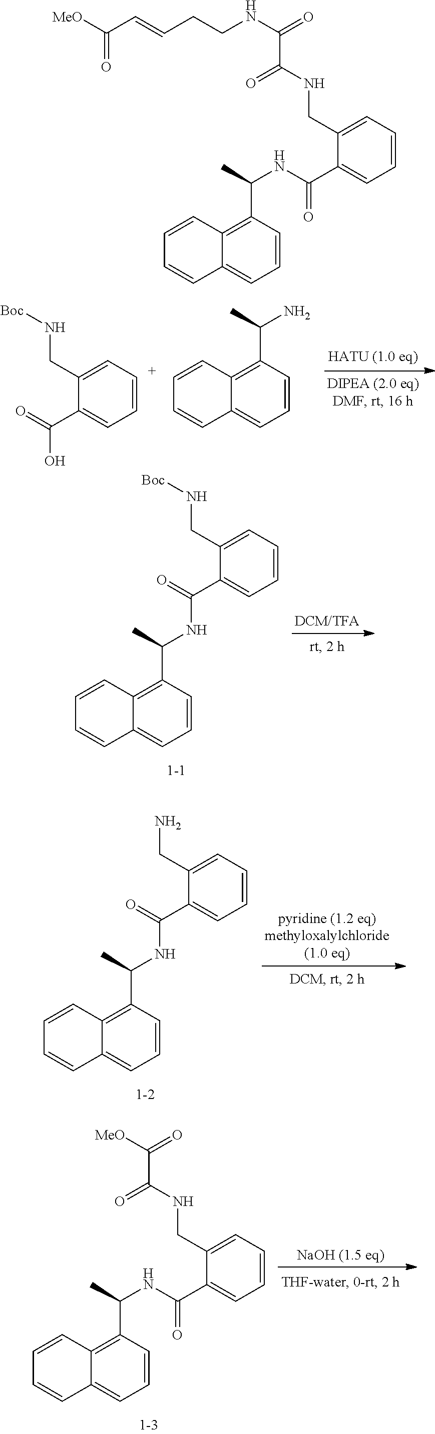

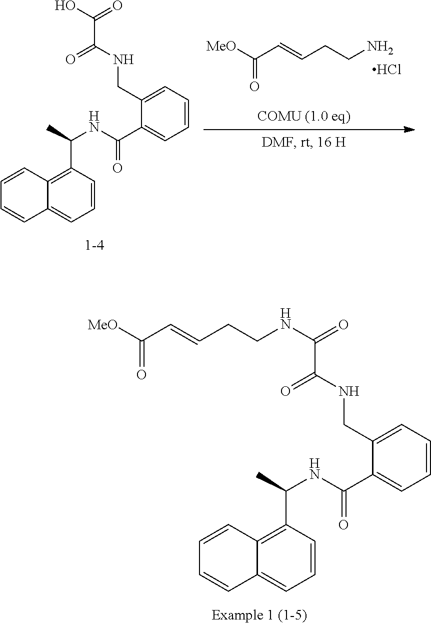 [SMILES: C[C@@H](NC(=O)c1ccccc1CNC(=O)C(=O)O)c1cccc2ccccc12 ; COC(=O)/C=C/CCN ; Cl ; COC(=O)/C=C/CCNC(=O)C(=O)NCc1ccccc1C(=O)N[C@H](C)c1cccc2ccccc12]

### Step 1-1:

A mixture of 2-(Boc-aminomethyl)benzoic acid (1.00 eq, 503 mg, 2.00 mmol), (1R)-1-(1-naphthyl)ethanamine (1.10 eq, 377 mg, 2.20 mmol), DIPEA (2.50 eq, 0.87 mL, 5.00 mmol) and HATU (1.10 eq, 837 mg, 2.20 mmol) in DMF/DCM (2 mL/2 mL) was stirred at rt for 20 h. The mixture was then diluted with EtOAc, washed with water and brine, dried over Na₂SO₄, filtered, and concentrated on a column to give the desired product (720 mg, 1.78 mmol, 89% yield). ESI MS m/z=427.19 [M+Na]⁺.

### Step 1-2:

A mixture of the compound from Step 1-1 (1.00 eq, 720 mg, 1.78 mmol) in DCM/TFA (10/10 mL) was stirred at rt for 2 h. It was conc. diluted with DCM, basified with Na₂CO₃ solution, extracted with DCM, dry over Na₂SO₄, filtered, conc to give the desired product (500 mg, 1.64 mmol, 92% yield). ESI MS m/z=327.15 [M+Na]⁺.

### Step 1-3:

To a solution of pyridine (1.50 eq, 0.20 mL, 2.46 mmol) and the compound from Step 1-2 (1.00 eq, 500 mg, 1.64 mmol) in DCM (10 mL) at 0° C. was treated with methyl oxalyl chloride (1.00 eq, 0.15 mL, 1.64 mmol), after 1 h. it was quenched with water, extracted with DCM, washed with 1N HCl, followed by a mixture of NaHCO₃ and brine, dried over Na₂SO₄, filtered, and concentrated to give the desired product (500 mg, 1.28 mmol, 78% yield). ESI MS m/z=413.15 [M+Na]⁺.

### Step 1-4:

To a mixture of the compound from Step 1-3 (1.00 eq, 480 mg, 1.23 mmol) in THE (4 mL) at 0° C. was treated with 1N NaOH (1.50 eq, 1.8 mL, 1.84 mmol), after 2 h, it was treated with 1 N HCl to PH=3, extracted with EtOAc, filtered, and concentrated to give the desired product (450 mg, 1.20 mmol, 97% yield). ESI MS m/z=377.29 [M+H]⁺.

### Step 1-5:

A mixture of the compound from Step 1-4 (1.00 eq, 75 mg, 0.200 mmol), DIPEA (3.50 eq, 0.12 mL, 0.700 mmol), methyl (E)-5-aminopent-2-enoate hydrochloride (1.00 eq, 33 mg, 0.200 mmol) and COW (1.00 eq, 86 mg, 0.200 mmol) in DMF (1.5 mL) was stirred at rt for 16 h. It was diluted with DMSO and purified with prep-HPLC to give Example 1 (22 mg, 0.0451 mmol, 23% yield). ESI MS m/z=488.22 [M+H]⁺.

### Example 436

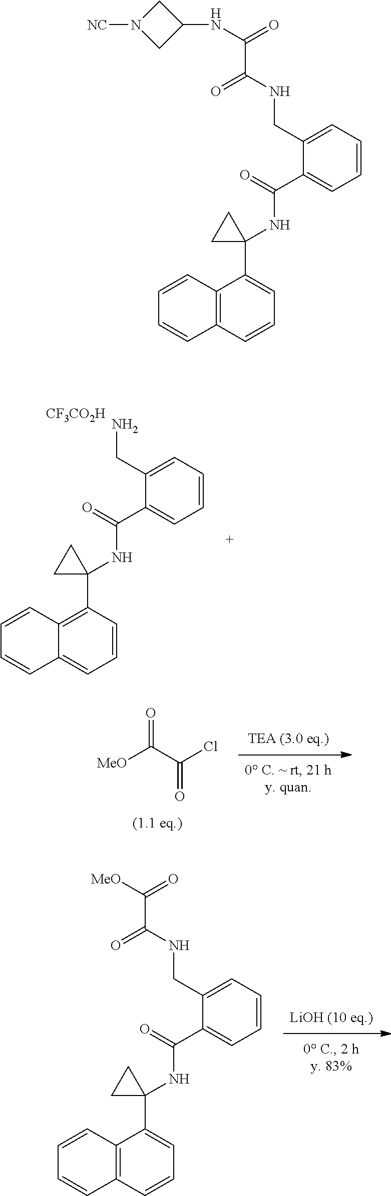 [SMILES: N#CN1CC(NC(=O)C(=O)NCc2ccccc2C(=O)NC2(c3cccc4ccccc34)CC2)C1 ; NCc1ccccc1C(=O)NC1(c2cccc3ccccc23)CC1 ; "CF3CO2H" ; COC(=O)C(=O)Cl ; COC(=O)C(=O)NCc1ccccc1C(=O)NC1(c2cccc3ccccc23)CC1]

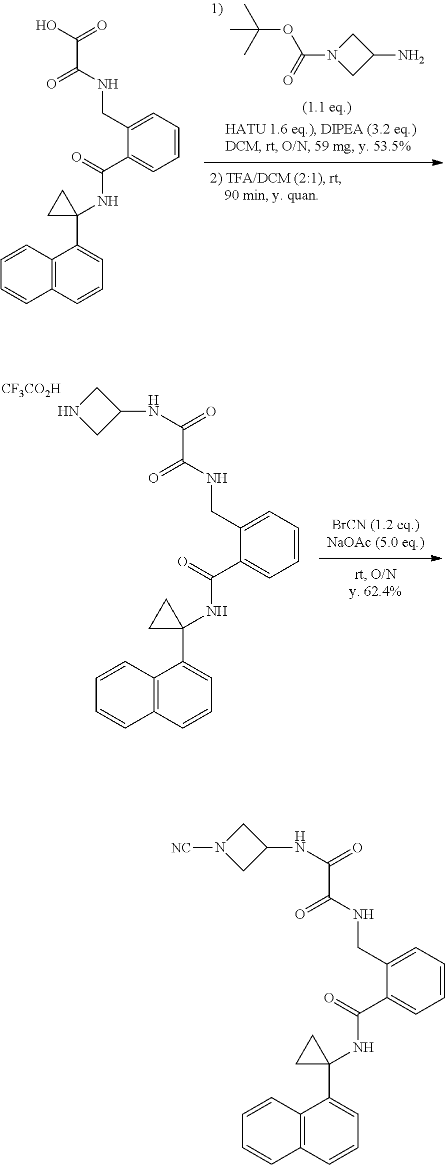 [SMILES: O=C(O)C(=O)NCc1ccccc1C(=O)NC1(c2cccc3ccccc23)CC1 ; CC(C)(C)OC(=O)N1CC(N)C1 ; "CF3CO2H" ; O=C(NCc1ccccc1C(=O)NC1(c2cccc3ccccc23)CC1)C(=O)NC1CNC1 ; N#CN1CC(NC(=O)C(=O)NCc2ccccc2C(=O)NC2(c3cccc4ccccc34)CC2)C1]

To a 40 mL vial containing 2-(aminomethyl)-N—(1-(naphthalen-1-yl)cyclopropyl)benzamide TFA salt (211 mg, 0.490 mmol) were added DCM (4.9 mL) and the solution was cooled to 0° C. followed by addition of TEA (205 μL, 1.471 mmol) and methyl 2-chloro-2-oxoacetate (49.6 μL, 0.539 mmol). After stirred at 0° C. ˜rt for 21 h, the rxn was diluted with DCM. Washed with half-brine, dried, filtered, concentrated to give methyl 2-((2-((1-(naphthalen-1-yl)cyclopropyl)carbamoyl)benzyl)amino)-2-oxoacetate (203 mg, quan. yield) as an off-white solid. Used directly without purification, (M+H)⁺: 403.17.

To a 50 mL round-bottomed flask containing methyl 2-((2-((1-(naphthalen-1-yl)cyclopropyl)carbamoyl)benzyl)amino)-2-oxoacetate (197 mg, 0.490 mmol) was added THE (6.53 mL) and the solution was cooled to 0° C. followed by addition of a solution of LiOH (117 mg, 4.90 mmol) in water (3.26 mL) slowly. After stirred at 0° C. for 2 h, slowly quenched by 3 N HCl (1632 μL, 4.90 mmol). The suspension was diluted with DCM, washed with brine, dried, filtered, concentrated to give 2-((2-((1-(naphthalen-1-yl)cyclopropyl)carbamoyl)benzyl)amino)-2-oxoacetic acid (158 mg, 83% yield) as an off-white solid, (M+H)⁺: 389.15.

To a 2-dram vial containing 2-((2-((1-(naphthalen-1-yl)cyclopropyl)carbamoyl)benzyl)amino)-2-oxoacetic acid (79 mg, 0.203 mmol) were added tert-butyl 3-aminoazetidine-1-carboxylate (33.5 μL, 0.224 mmol), HATU (124 mg, 0.325 mmol), DCM (1.0 mL), and Hunig's base (114 μL, 0.651 mmol) respectively and the suspension was stirred at rt overnight. Diluted with DCM, washed with 10% citric acid, Sat. NaHCO₃, and brine respectively. Dried, filtered, concentrated and purified by CombiFlash (Ace/c-Hex: 0˜50%) to give tert-butyl 3-(2-((2-((1-(naphthalen-1-yl)cyclopropyl)carbamoyl)benzyl)amino)-2-oxoacetamido)azetidine-1-carboxylate (59 mg, 0.109 mmol, 53.5% yield) as a colorless oil, (M−H)⁻: 541.24.

To a 40 mL vial containing tert-butyl 3-(2-((2-((1-(naphthalen-1-yl)cyclopropyl)carbamoyl)benzyl)amino)-2-oxoacetamido)azetidine-1-carboxylate (59 mg, 0.109 mmol) were added DCM (1.5 mL) and TFA (3 mL) and the solution was stirred at rt for 90 min. Concentrated to give N1-(azetidin-3-yl)-N2-(2-((1-(naphthalen-1-yl)cyclopropyl)carbamoyl)benzyl)oxalamide as an off-white powder (61 mg, y. quan.), (M+H)⁺: 443.21.

To a 1-dram vial were added N1-(azetidin-3-yl)-N2-(2-((1-(naphthalen-1-yl)cyclopropyl)carbamoyl)benzyl)oxalamide (14.5 mg, 0.026 mmol), sodium acetate (10.69 mg, 0.130 mmol), DMF (0.5 mL), and cyanic bromide in ACN (6.25 μL, 0.031 mmol) respectively and the solution was stirred at rt overnight. Without workup, diluted into DMSO, and purified by Prep HPLC (40˜80% ACN/water over 35 min) to give N1-(1-cyanoazetidin-3-yl)-N2-(2-((1-(naphthalen-1-yl)cyclopropyl)carbamoyl)benzyl)oxalamide as Example 436, (7.6 mg, 62.4% yield) as a white solid, (M−H)⁻: 466.19.

### Example 441

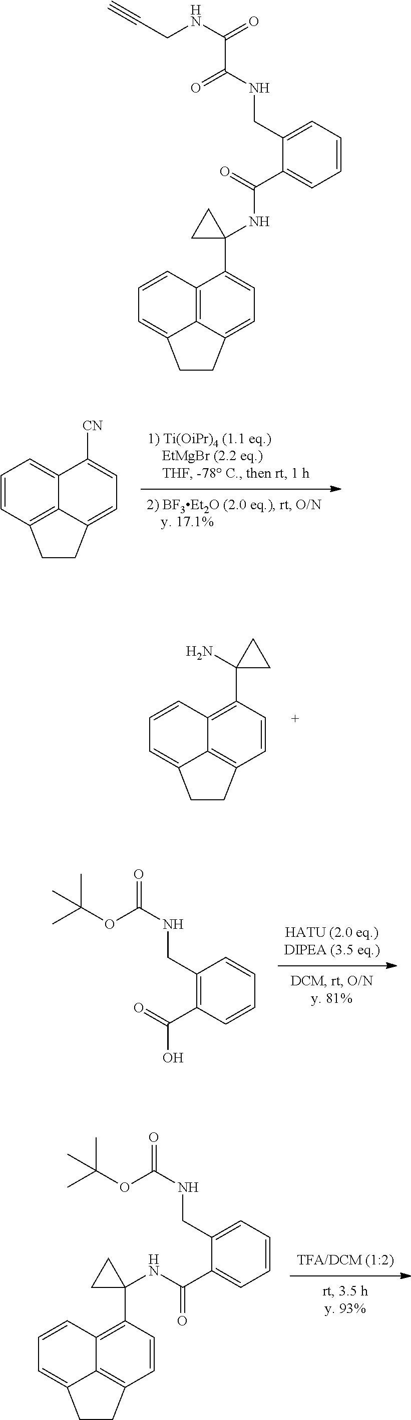 [SMILES: C#CCNC(=O)C(=O)NCc1ccccc1C(=O)NC1(c2ccc3c4c(cccc24)CC3)CC1 ; N#Cc1ccc2c3c(cccc13)CC2 ; NC1(c2ccc3c4c(cccc24)CC3)CC1 ; CC(C)(C)OC(=O)NCc1ccccc1C(=O)O ; CC(C)(C)OC(=O)NCc1ccccc1C(=O)NC1(c2ccc3c4c(cccc24)CC3)CC1]

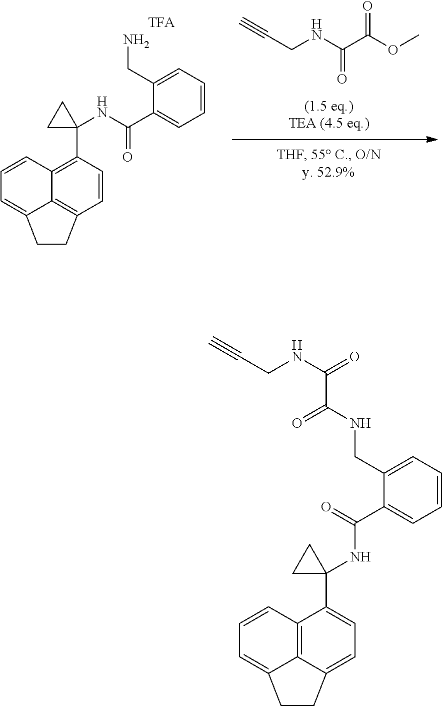

To a 100 mL round-bottomed flask were added 1,2-dihydroacenaphthylene-5-carbonitrile (1 g, 5.58 mmol) and THE (18.6 mL) and the solution was cooled to −78° C. under N₂. Tetraisopropoxytitanium (1.8 mL, 6.14 mmol) was added quickly followed by the addition of ethylmagnesium bromide in 2-MeTHF (3.84 mL, 12.28 mmol) dropwise. Stirred at −78° C. for 70 min and then the suspension was stirred at rt for 1 h. BF₃·OEt₂ (2.1 mL, 16.74 mmol) was added dropwise at rt and the reaction was stirred at rt overnight. Cooled to 0° C., quenched with 25 mL of 1 N HCl. The mixture was diluted with 50 mL of water and then extracted with MTBE (50 mL×2). The aqueous layers were combined and cooled to 0° C. followed by the addition of 25 mL of 2 N NaOH dropwise. The mixture was dissolved in 350 mL of DCM, washed with brine and dried over Na₂SO₄. Filtered and concentrated to give 1-(1,2-dihydroacenaphthylen-5-yl)cyclopropan-1-amine (200 mg, 17.1% yield) as an orange oil, (M-NH₂)⁺: 193.10.

To a 40 mL vial containing 1-(1,2-dihydroacenaphthylen-5-yl)cyclopropan-1-amine (150 mg, 0.717 mmol) were added 2-(((tert-butoxycarbonyl)amino)methyl)benzoic acid (216 mg, 0.860 mmol)), HATU (545 mg, 1.433 mmol), DCM (3.58 mL), and DIPEA (438 μL, 2.508 mmol) respectively and the yellow solution was stirred at rt for 20 h. Diluted with DCM, washed with 10% citric acid, Sat. NaHCO₃, and brine respectively. Dried, filtered, concentrated and purified by CombiFlash (EA/c-Hex: 0˜30%) to give tert-butyl (2-((1-(1,2-dihydroacenaphthylen-5-yl)cyclopropyl)carbamoyl)benzyl)carbamate (258.2 mg, 81% yield) as a colorless oil, (M+Na)⁺: 465.21.

To a 20 mL vial containing tert-butyl (2-((1-(1,2-dihydroacenaphthylen-5-yl)cyclopropyl)carbamoyl)benzyl)carbamate (258.2 mg, 0.583 mmol) were added DCM (3 mL) and TFA (1.5 mL, 19.47 mmol) and the light brown solution was stirred at rt for 3.5 h. Concentrated to give 2-(aminomethyl)-N-(1-(1,2-dihydroacenaphthylen-5-yl)cyclopropyl)benzamide TFA salt (247 mg, 93% yield) as an off-white powder, (M+H)⁺: 343.18.

To a 1-dram vial were added 2-(aminomethyl)-N-(1-(1,2-dihydroacenaphthylen-5-yl)cyclopropyl)benzamide TFA salt (30 mg, 0.066 mmol), methyl 2-oxo-2-(prop-2-yn-1-ylamino)acetate (13.91 mg, 0.099 mmol), THE (1 mL), and triethylamine (41.2 μL, 0.296 mmol) respectively and the solution was heated at 55° C. overnight. The solution was concentrated and purified by CombiFlash (Ace/c-Hex: 0˜100%) to give N1-(2-((1-(1,2-dihydroacenaphthylen-5-yl)cyclopropyl)carbamoyl)benzyl)-N2-(prop-2-yn-1-yl)oxalamide as Example 441, (15.7 mg, 52.9% yield) as a white solid, (M−H)⁻: 450.18.

### Example 457

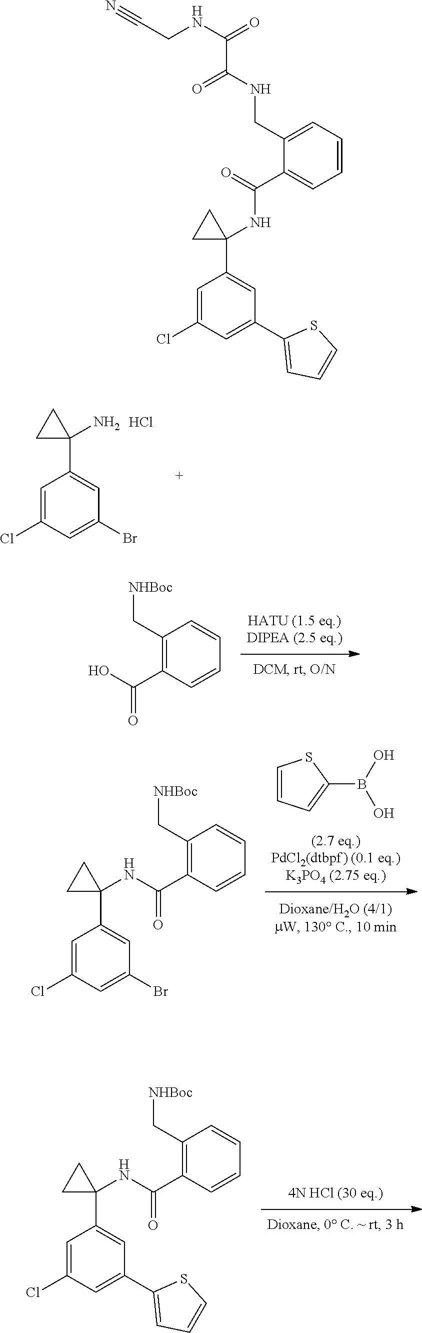

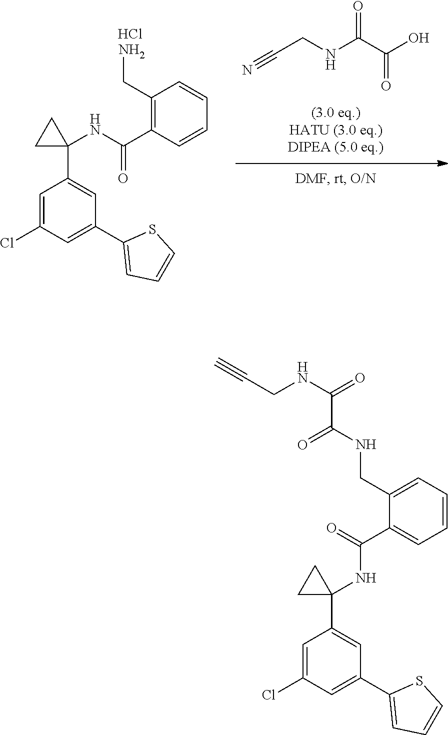 [SMILES: NCc1ccccc1C(=O)NC1(c2cc(Cl)cc(-c3cccs3)c2)CC1 ; Cl ; N#CCNC(=O)C(=O)O ; C#CCNC(=O)C(=O)NCc1ccccc1C(=O)NC1(c2cc(Cl)cc(-c3cccs3)c2)CC1]

To a 100 mL round-bottomed flask were added 1-(3-bromo-5-chlorophenyl)cyclopropan-1-amine hydrochloride (1.92 g, 6.77 mmol), 2-(((tert-butoxycarbonyl)amino)methyl)benzoic acid (1.7 g, 6.77 mmol), HATU (3.86 g, 10.15 mmol), DCM (22.6 mL) respectively and the suspension was cooled to 0° C. under N₂ followed by addition of DIPEA (2.95 mL, 16.91 mmol). The rxn was stirred at 0° C.˜rt overnight. Diluted with DCM, washed with 10% citric acid, sat. NaHCO₃ and brine respectively. The organic layer was dried, filtered, concentrated, purified by CombiFlash (Ace/c-Hex: 0˜50%) to give tert-butyl (2-((1-(3-bromo-5-chlorophenyl)cyclopropyl)carbamoyl)benzyl)carbamate (1.9 g, 58.5% yield) as a light orange solid, (M−H)⁻: 477.06.

To a 5 mL microwave vial were added thiophen-2-ylboronic acid (144 mg, 1.125 mmol), tert-butyl (2-((1-(3-bromo-5-chlorophenyl)cyclopropyl)carbamoyl)benzyl)carbamate (200 mg, 0.417 mmol), K₃PO₄ (243 mg, 1.146 mmol), and PdCl₂(dtbpf) (27.2 mg, 0.042 mmol) respectively and the flask was sealed. After internal atmosphere was switched to N2, Dioxane (3.33 mL) and Water (0.834 mL) were added and the suspension was microwave irradiated at 130° C. for 10 min. Diluted with EtOAc, washed with water and brine. Dried, filtered, and concentrated to give a brown residue. Purified by CombiFlash (Ace/c-Hex: 0˜30%) to give tert-butyl (2-((1-(3-chloro-5-(thiophen-2-yl)phenyl)cyclopropyl)carbamoyl)benzyl)carbamate (63.1 mg, 31.3% yield) as a white solid, (M−H)⁻: 481.14.

To a 2-dram vial were added a solution of tert-butyl (2-((1-(3-chloro-5-(thiophen-2-yl)phenyl)cyclopropyl)carbamoyl)benzyl)carbamate (63.1 mg, 0.131 mmol) in DCM (2 mL) and the solution was cooled to 0° C. followed by addition of 4 N HCl in dioxane (980 μL, 3.9 mmol). The rxn was stirred at rt for 1 h and then concentrated in vacuo to give 2-(aminomethyl)-N-(1-(3-chloro-5-(thiophen-2-yl)phenyl)cyclopropyl)benzamide hydrochloride (54.7 mg, 100% yield) as a yellow powder, (M+H)⁺: 383.10.

To a 25 mL round-bottomed flask containing 2-(aminomethyl)-N—(1-(3-chloro-5-(thiophen-2-yl)phenyl)cyclopropyl)benzamide hydrochloride (0.146 g, 0.349 mmol) were added methyl 2-((cyanomethyl)amino)-2-oxoacetate (0.064 g, 0.454 mmol), THE (3.49 mL), and triethylamine (0.219 mL, 1.571 mmol) respectively and the solution was stirred at rt for 17 h. The reaction mixture was diluted with DCM and washed with half brine. The organic layer was dried, filtered, and concentrated. The crude product was purified by Prep HPLC (ACN/water, 40˜80%, 35 min) to give N1-(2-((1-(3-chloro-5-(thiophen-2-yl)phenyl)cyclopropyl)carbamoyl)benzyl)-N2-(cyanomethyl)oxalamide (47.4 mg, 27.6% yield) as Example 457, as a white solid, (M+H)⁺: 493.11.

### Example 518

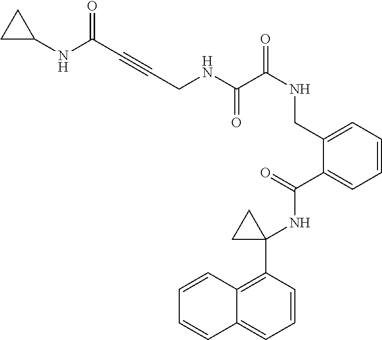 [SMILES: O=C(C#CCNC(=O)C(=O)NCc1ccccc1C(=O)NC1(c2cccc3ccccc23)CC1)NC1CC1]

Step 1: To a solution of 4-((tert-butoxycarbonyl)amino)but-2-ynoic acid (102 mg, 0.514 mmol) and cyclopropanamine (44 mg, 0.771 mmol) in DMF (5.14 ml) at rt was added iPr₂NEt (269 μl, 1.541 mmol) and HATU (195 mg, 0.514 mmol). Then the reaction mixture was stirred at rt for 16 hours. The reaction was quenched with NaHCO₃ aqueous solution, and the reaction mixture was extracted with EtOAc for 3 times. The combined organic phases were washed with water and brine, dried over Na₂SO₄, concentrated. The resulting crude product was purified by flash column chromatography with the eluent of EtOAc/hexanes to give the desired product (61 mg, 50%).). ESI MS m/z=261.10 [M+Na]⁺.

Step 2 To a solution of the Compound from Step 1 (61 mg, 0.256 mmol) in CH₂Cl₂ (1.280 ml) at 0° C. was added TFA (986 μl, 12.80 mmol), then the reaction mixture was stirred at 0° C. for 15 minutes, then concentrated to give the desired product without further purification.). ESI MS m/z=139.09 [M+H]⁺.

Step 3 To a solution of 2-((2-((1-(naphthalen-1-yl)cyclopropyl)carbamoyl)benzyl)amino)-2-oxoacetic acid (0.099 g, 0.256 mmol) and the Compound from Step 2 (0.045 g, 0.256 mmol) in DMF (2.56 ml) at rt was added iPr₂NEt (0.134 ml, 0.768 mmol) and HATU (0.097 g, 0.256 mmol). Then the reaction mixture was stirred at rt for 16 hours. The reaction was quenched with NaHCO₃ aqueous solution, and the reaction mixture was extracted with EtOAc for 3 times. The combined organic phases were washed with water and brine, dried over Na₂SO₄, concentrated. The resulting crude product was purified by flash column chromatography with the eluent of EtOAc/hexanes to give the desired product (13.3 mg, 10%). ESI MS m/z=507.30 [M−H]⁻. ¹H NMR (400 MHz, MeOD) δ 8.62 (d, J=8.5 Hz, 1H), 7.96-7.88 (m, 2H), 7.82 (d, J 8.3 Hz, 1H), 7.65-7.55 (m, 1H), 7.53-7.43 (m, 2H), 7.41-7.35 (m, 1H), 7.31 (d, J=7.5 Hz, 1H), 7.30-7.24 (m, 1H), 7.24-7.19 (m, 1H), 6.19 (d, J=1.4 Hz, 1H), 4.28 (s, 2H), 4.11 (d, J=1.4 Hz, 2H), 1.58-1.41 (m, 3H), 1.37-1.29 (m, 2H), 0.76 (td, J=7.1, 5.1 Hz, 2H), 0.60-0.41 (m, 2H).

### Example 538

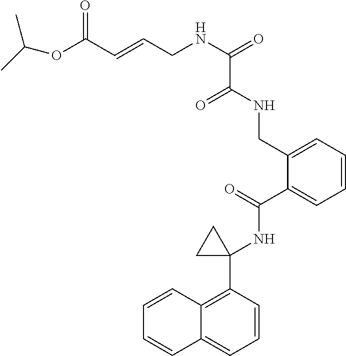 [SMILES: CC(C)OC(=O)/C=C/CNC(=O)C(=O)NCc1ccccc1C(=O)NC1(c2cccc3ccccc23)CC1]

Step 1. To a stirred solution of tert-butyl (2-oxoethyl)carbamate (1.6 g, 10.051 mmol, 1 equiv) and methyl 2-(dimethoxyphosphoryl)acetate (2.75 g, 15.076 mmol, 1.5 equiv) in THE (40 mL) at 0° C. was added NaH (0.48 g, 20.102 mmol, 2 equiv) in portions. The solution was stirred for 2 h at 0° C., quenched with saturated NH₄Cl solution, extracted with EtOAc (3×40 mL). The combined organic phases were washed with brine, dried with Na₂SO₄ and concentrated under vacuum to give the desired crude product (2 g, 92.44%) as a yellow oil, which was used in the next step directly without further purification.

Step 2. A solution of the Compound from Step 1 (2 g, 9.292 mmol, 1 equiv) in 4N HCl/1,4-dioxane (20 mL) was stirred at rt for 1 h. The reaction solution was concentrated under vacuum to give the desired product (1.0 g, 93.48%) as a brown solid, which was used in the next step directly without further purification.

Step 3. To a solution of 2-((2-((1-(naphthalen-1-yl)cyclopropyl)carbamoyl)benzyl)amino)-2-oxoacetic acid (1 g, 2.575 mmol, 1 equiv), the Compound from Step 2 (0.30 g, 2.575 mmol, 1 equiv) in DCM (8 mL) and DMF (8 mL) at rt was added PyBOP (1.47 g, 2.833 mmol, 1.1 equiv) and iPr₂NEt (1.00 g, 7.725 mmol, 3 equiv). Then the reaction mixture was stirred at rt for 1 h. The reaction solution was concentrated under vacuum and the residue was purified by reversed-phase flash chromatography with C18 silica gel column and H₂O/MeCN as eluents to give the desired product (1.0 g, 80.00%) as a yellow solid. (ES, m/z): [M+H]⁺=486.15

Step 4 To a solution of the compound from Step 3 (250 mg, 0.515 mmol, 1 equiv) in MeOH (6 mL) and H₂O (3 mL) at rt was added NaOH (61.78 mg, 1.545 mmol, 3 equiv).l Then the reaction mixture was stirred at rt for 30 min. The solution was concentrated under vacuum to MeOH and the residue was acidified to pH 3 with conc. HCl solution. The mixture was concentrated under vacuum to give the desired product (230 mg, 94.74%) as an off-white solid, which was used in the next step directly without further purification. (ES, m/z): [M+H]⁺=472.10.

Step 5 To a stirred solution of the compound from Step 4 (100 mg, 0.212 mmol, 1 equiv), EDCI (60.98 mg, 0.318 mmol, 1.5 equiv) and DMAP (2.59 mg, 0.021 mmol, 0.1 equiv) in DCM (2 mL) and DMF (2 mL) at rt was added 2-propanol (12.75 mg, 0.212 mmol, 1 equiv) and DIEA (82.23 mg, 0.636 mmol, 3 equiv) dropwise. The solution was stirred overnight at rt and then concentrated under vacuum. The residue was purified Prep-HPLC to give the title product (3.0 mg, 2.72%) as a white solid. (ES, m/z): [M+Na]⁺=536.25. ¹H NMR (400 MHz, DMSO-d6) δ 10.01 (d, J=10.5 Hz, 1H). 9.35 (s, 1H), 9.09 (t, J=6.4 Hz, 1H), 8.68 (d, J=8.4 Hz, 1H), 7.79-8.00 (m, 3H), 7.15-7.64 (m, 7H), 6.61 (tt, J=10.5, 1.7 Hz, 1H), 4.81-5.13 (m, 2H), 4.29 (d, J=6.4 Hz, 2H), 3.27 (dd, J=7.4, 1.7 Hz, 2H), 1.41 (q, J=4.7, 4.3 Hz, 2H), 1.18 (d, J=6.3 Hz, 8H).

TABLE 1
The following examples were prepared employing methods similar to those described above.

| Example | Structure | MS [M + H]⁺ |
|---|---|---|
| 2 | 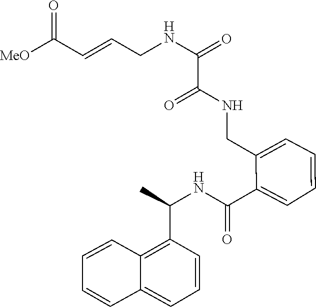 [SMILES: COC(=O)/C=C/CNC(=O)C(=O)NCc1ccccc1C(=O)N[C@H](C)c1cccc2ccccc12] | 474.24 |
| 3 | 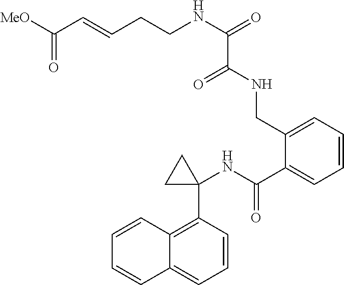 [SMILES: COC(=O)/C=C/CCNC(=O)C(=O)NCc1ccccc1C(=O)NC1(c2cccc3ccccc23)CC1] | 500.44 |
| 4 | 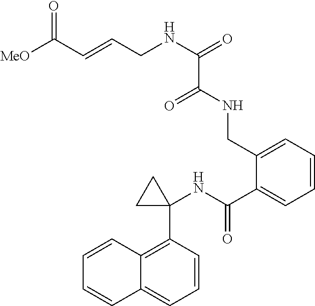 [SMILES: COC(=O)/C=C/CNC(=O)C(=O)NCc1ccccc1C(=O)NC1(c2cccc3ccccc23)CC1] | 486.41 |
| 5 | 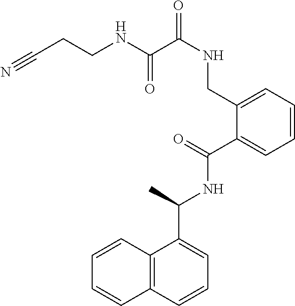 [SMILES: C[C@@H](NC(=O)c1ccccc1CNC(=O)C(=O)NCCC#N)c1cccc2ccccc12] | 427.07 [M − H]⁻ |
| 6 | 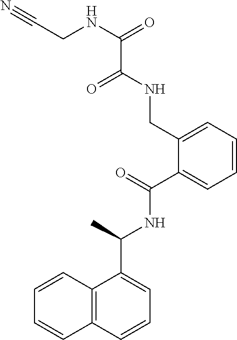 [SMILES: C[C@@H](NC(=O)c1ccccc1CNC(=O)C(=O)NCC#N)c1cccc2ccccc12] | 415.10 |
| 7 | 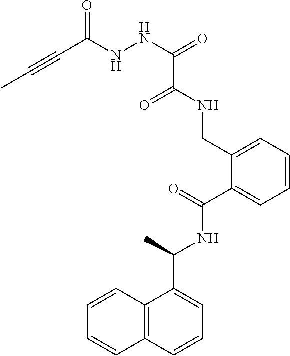 [SMILES: CC#CC(=O)NNC(=O)C(=O)NCc1ccccc1C(=O)N[C@H](C)c1cccc2ccccc12] | 457.11 |
| 8 | 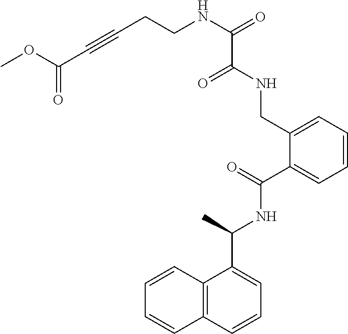 [SMILES: COC(=O)C#CCCNC(=O)C(=O)NCc1ccccc1C(=O)N[C@H](C)c1cccc2ccccc12] | 486.13 |
| 9 | 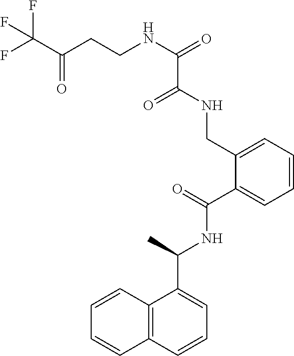 [SMILES: C[C@@H](NC(=O)c1ccccc1CNC(=O)C(=O)NCCC(=O)C(F)(F)F)c1cccc2ccccc12] | 501.22 |
| 10 | 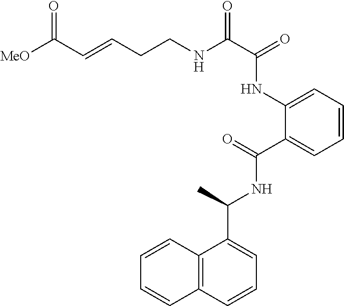 [SMILES: COC(=O)/C=C/CCNC(=O)C(=O)Nc1ccccc1C(=O)N[C@H](C)c1cccc2ccccc12] | 474.13 |
| 11 | 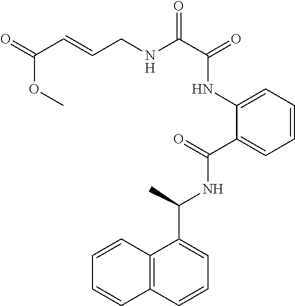 [SMILES: COC(=O)/C=C/CNC(=O)C(=O)Nc1ccccc1C(=O)N[C@H](C)c1cccc2ccccc12] | 460.12 |
| 12 | 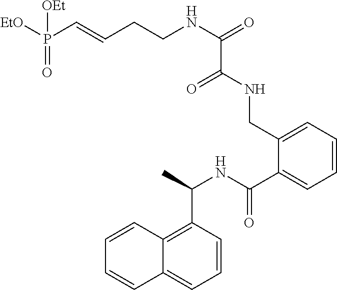 [SMILES: CCOP(=O)(/C=C/CCNC(=O)C(=O)NCc1ccccc1C(=O)N[C@H](C)c1cccc2ccccc12)OCC] | 566.44 |
| 13 | 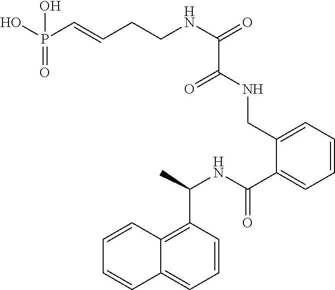 [SMILES: C[C@@H](NC(=O)c1ccccc1CNC(=O)C(=O)NCC/C=C/P(=O)(O)O)c1cccc2ccccc12] | 510.28 |
| 14 | 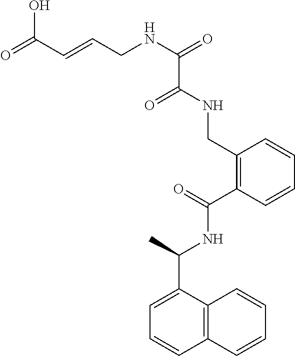 [SMILES: C[C@@H](NC(=O)c1ccccc1CNC(=O)C(=O)NC/C=C/C(=O)O)c1cccc2ccccc12] | 460.20 |
| 15 | 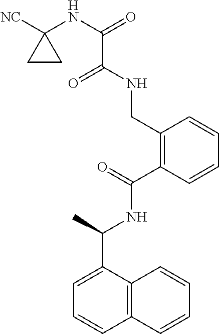 [SMILES: C[C@@H](NC(=O)c1ccccc1CNC(=O)C(=O)NC1(C#N)CC1)c1cccc2ccccc12] | 441.20 |
| 16 | 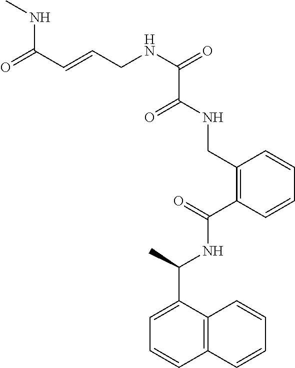 [SMILES: CNC(=O)/C=C/CNC(=O)C(=O)NCc1ccccc1C(=O)N[C@H](C)c1cccc2ccccc12] | 473.22 |
| 17 | 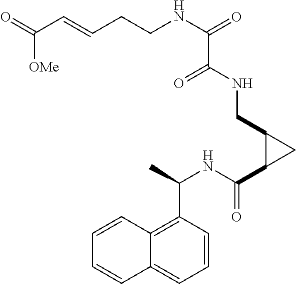 | 452.14 |
| 18 | 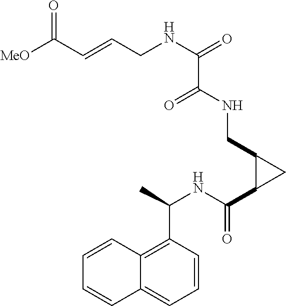 | 438.21 |
| 19 | 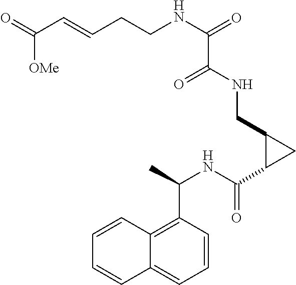 | 452.14 |
| 20 | 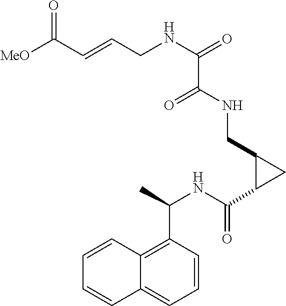 | 438.21 |
| 21 | 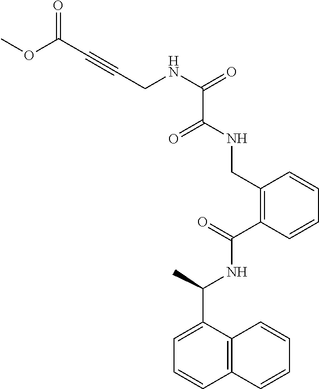 [SMILES: COC(=O)C#CCNC(=O)C(=O)NCc1ccccc1C(=O)N[C@H](C)c1cccc2ccccc12] | 473.40 |
| 22 | 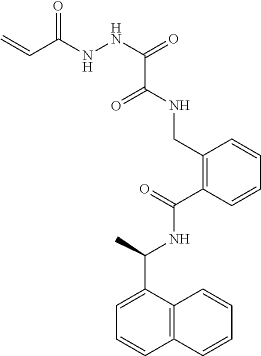 [SMILES: C=CC(=O)NNC(=O)C(=O)NCc1ccccc1C(=O)N[C@H](C)c1cccc2ccccc12] | 445.19 |
| 23 | 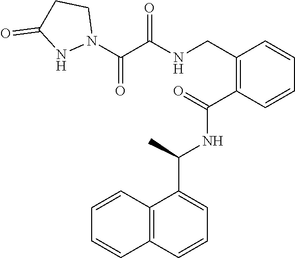 [SMILES: C[C@@H](NC(=O)c1ccccc1CNC(=O)C(=O)N1CCC(=O)N1)c1cccc2ccccc12] | 445.19 |
| 24 | 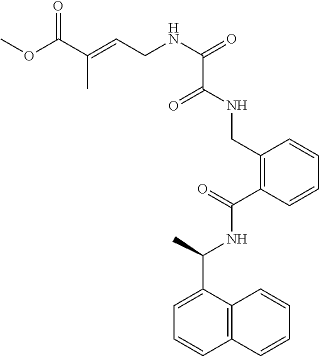 [SMILES: COC(=O)/C(C)=C/CNC(=O)C(=O)NCc1ccccc1C(=O)N[C@H](C)c1cccc2ccccc12] | 488.23 |
| 25 | 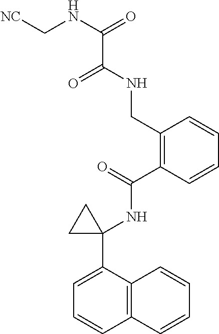 [SMILES: N#CCNC(=O)C(=O)NCc1ccccc1C(=O)NC1(c2cccc3ccccc23)CC1] | 427.19 |
| 26 | 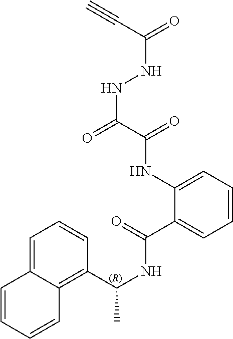 | 427.05 [M − H]⁻ |
| 27 | 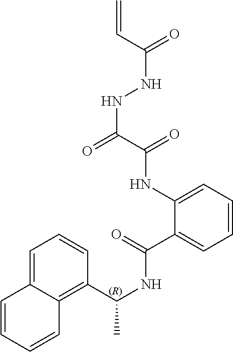 | 431.15 |
| 28 | 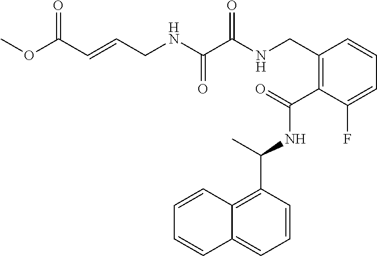 [SMILES: COC(=O)/C=C/CNC(=O)C(=O)NCc1cccc(F)c1C(=O)N[C@H](C)c1cccc2ccccc12] | 492.33 |
| 29 | 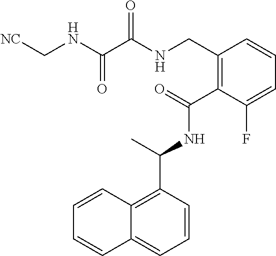 [SMILES: C[C@@H](NC(=O)c1c(F)cccc1CNC(=O)C(=O)NCC#N)c1cccc2ccccc12] | 433.27 |
| 30 | 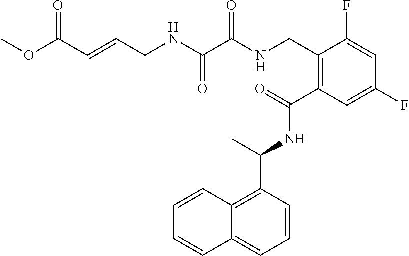 [SMILES: COC(=O)/C=C/CNC(=O)C(=O)NCc1c(F)cc(F)cc1C(=O)N[C@H](C)c1cccc2ccccc12] | 510.37 |
| 31 | 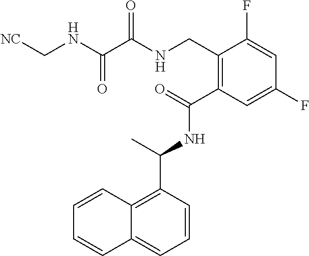 [SMILES: C[C@@H](NC(=O)c1cc(F)cc(F)c1CNC(=O)C(=O)NCC#N)c1cccc2ccccc12] | 451.29 |
| 32 | 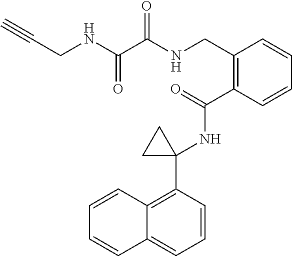 [SMILES: C#CCNC(=O)C(=O)NCc1ccccc1C(=O)NC1(c2cccc3ccccc23)CC1] | 426.33 |
| 33 | 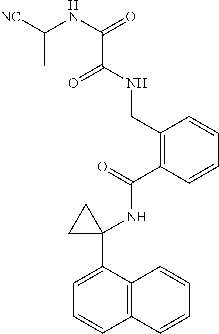 [SMILES: CC(C#N)NC(=O)C(=O)NCc1ccccc1C(=O)NC1(c2cccc3ccccc23)CC1] | 441.20 |
| 34 | 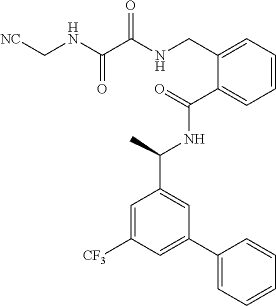 [SMILES: C[C@@H](NC(=O)c1ccccc1CNC(=O)C(=O)NCC#N)c1cc(-c2ccccc2)cc(C(F)(F)F)c1] | 507.54 [M − H]⁻ |
| 35 | 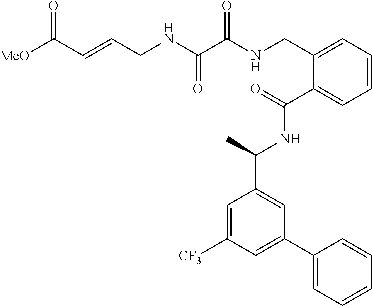 [SMILES: COC(=O)/C=C/CNC(=O)C(=O)NCc1ccccc1C(=O)N[C@H](C)c1cc(-c2ccccc2)cc(C(F)(F)F)c1] | 566.14 [M − H]⁻ |
| 36 | 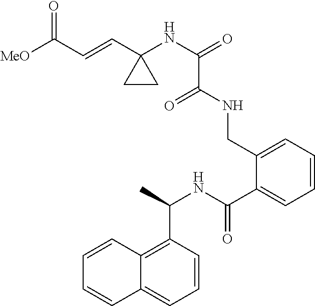 [SMILES: COC(=O)/C=C/C1(NC(=O)C(=O)NCc2ccccc2C(=O)N[C@H](C)c2cccc3ccccc23)CC1] | 500.20 |
| 37 | 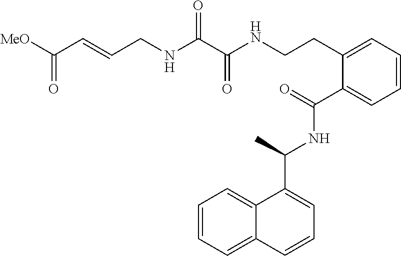 [SMILES: COC(=O)/C=C/CNC(=O)C(=O)NCCc1ccccc1C(=O)N[C@H](C)c1cccc2ccccc12] | 488.20 |
| 38 | 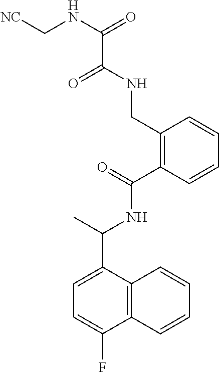 [SMILES: CC(NC(=O)c1ccccc1CNC(=O)C(=O)NCC#N)c1ccc(F)c2ccccc12] | 488.22 |
| 39 | 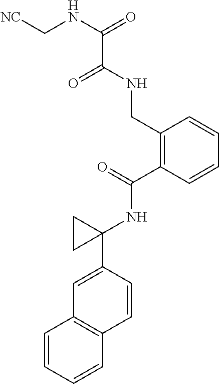 [SMILES: N#CCNC(=O)C(=O)NCc1ccccc1C(=O)NC1(c2ccc3ccccc3c2)CC1] | 427.18 |
| 40 | 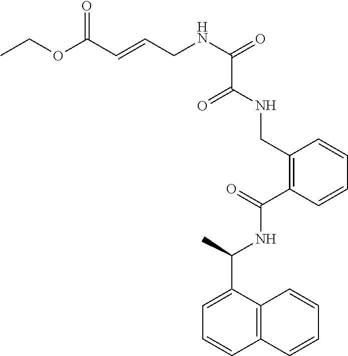 [SMILES: CCOC(=O)/C=C/CNC(=O)C(=O)NCc1ccccc1C(=O)N[C@H](C)c1cccc2ccccc12] | 433.17 |
| 41 | 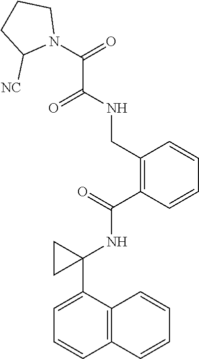 [SMILES: N#CC1CCCN1C(=O)C(=O)NCc1ccccc1C(=O)NC1(c2cccc3ccccc23)CC1] | 467.21 |
| 42 | 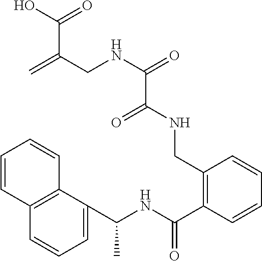 [SMILES: C=C(CNC(=O)C(=O)NCc1ccccc1C(=O)N[C@H](C)c1cccc2ccccc12)C(=O)O] | 460.19 |
| 43 | 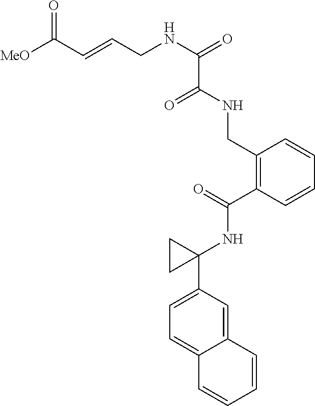 [SMILES: COC(=O)/C=C/CNC(=O)C(=O)NCc1ccccc1C(=O)NC1(c2ccc3ccccc3c2)CC1] | 486.21 |
| 44 | 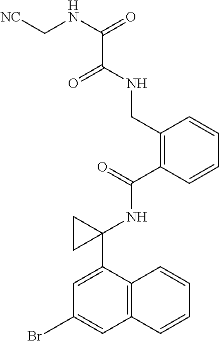 | 505.09 |
| 45 | 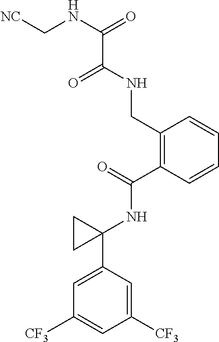 [SMILES: N#CCNC(=O)C(=O)NCc1ccccc1C(=O)NC1(c2cc(C(F)(F)F)cc(C(F)(F)F)c2)CC1] | 513.14 |
| 46 | 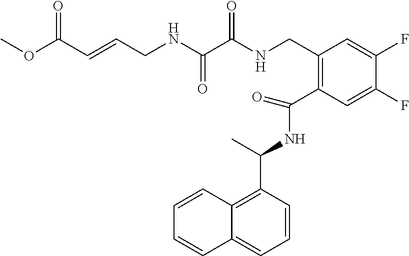 [SMILES: COC(=O)/C=C/CNC(=O)C(=O)NCc1cc(F)c(F)cc1C(=O)N[C@H](C)c1cccc2ccccc12] | 510.29 |
| 47 | 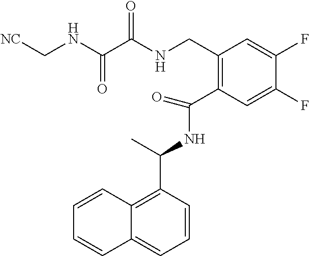 [SMILES: C[C@@H](NC(=O)c1cc(F)c(F)cc1CNC(=O)C(=O)NCC#N)c1cccc2ccccc12] | 451.24 |
| 48 | 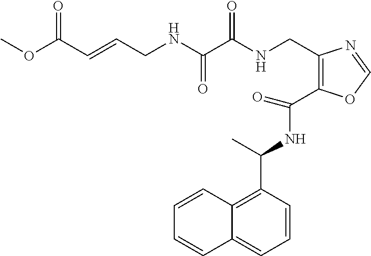 [SMILES: COC(=O)/C=C/CNC(=O)C(=O)NCc1ncoc1C(=O)N[C@H](C)c1cccc2ccccc12] | 465.26 |
| 49 | 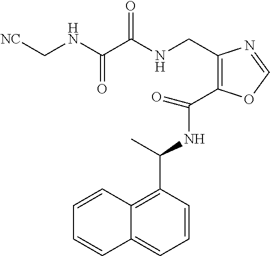 [SMILES: C[C@@H](NC(=O)c1ocnc1CNC(=O)C(=O)NCC#N)c1cccc2ccccc12] | 406.17 |
| 50 | 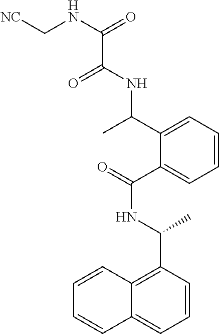 [SMILES: CC(NC(=O)C(=O)NCC#N)c1ccccc1C(=O)N[C@H](C)c1cccc2ccccc12] | 429.19 |
| 51 | 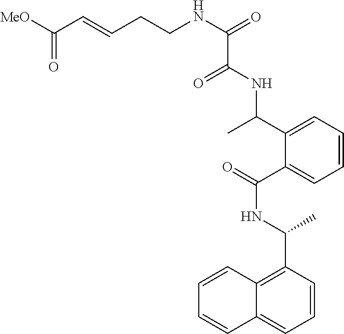 [SMILES: COC(=O)/C=C/CCNC(=O)C(=O)NC(C)c1ccccc1C(=O)N[C@H](C)c1cccc2ccccc12] | 502.26 |
| 52 | 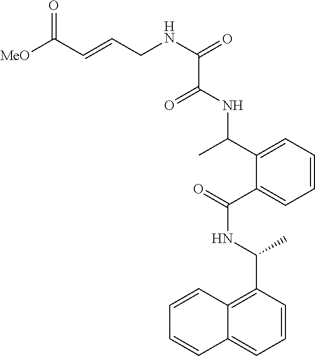 [SMILES: COC(=O)/C=C/CNC(=O)C(=O)NC(C)c1ccccc1C(=O)N[C@H](C)c1cccc2ccccc12] | 488.24 |
| 53 | 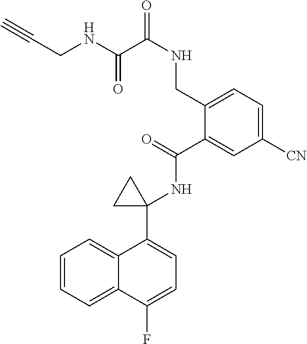 [SMILES: C#CCNC(=O)C(=O)NCc1ccc(C#N)cc1C(=O)NC1(c2ccc(F)c3ccccc23)CC1] | 469.04 |
| 54 | 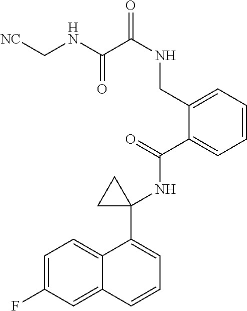 [SMILES: N#CCNC(=O)C(=O)NCc1ccccc1C(=O)NC1(c2cccc3cc(F)ccc23)CC1] | 445.00 |
| 55 | 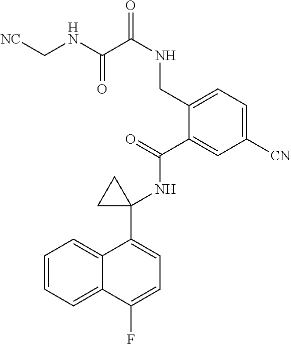 [SMILES: N#CCNC(=O)C(=O)NCc1ccc(C#N)cc1C(=O)NC1(c2ccc(F)c3ccccc23)CC1] | 469.96 |
| 56 | 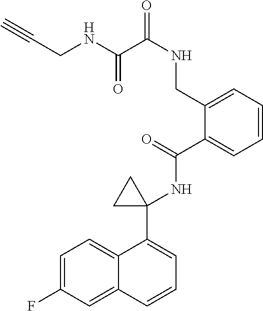 [SMILES: C#CCNC(=O)C(=O)NCc1ccccc1C(=O)NC1(c2cccc3cc(F)ccc23)CC1] | 444.04 |
| 57 | 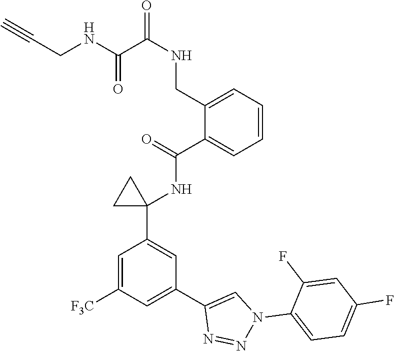 [SMILES: C#CCNC(=O)C(=O)NCc1ccccc1C(=O)NC1(c2cc(-c3cn(-c4ccc(F)cc4F)nn3)cc(C(F)(F)F)c2)CC1] | 623.23 |
| 58 | 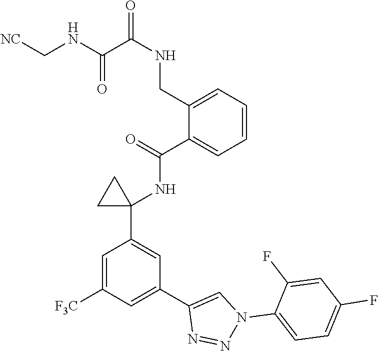 [SMILES: N#CCNC(=O)C(=O)NCc1ccccc1C(=O)NC1(c2cc(-c3cn(-c4ccc(F)cc4F)nn3)cc(C(F)(F)F)c2)CC1] | 624.19 |
| 59 | 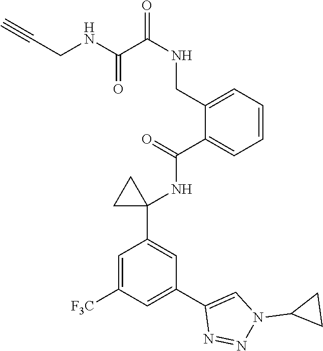 [SMILES: C#CCNC(=O)C(=O)NCc1ccccc1C(=O)NC1(c2cc(-c3cn(C4CC4)nn3)cc(C(F)(F)F)c2)CC1] | 551.17 |
| 60 | 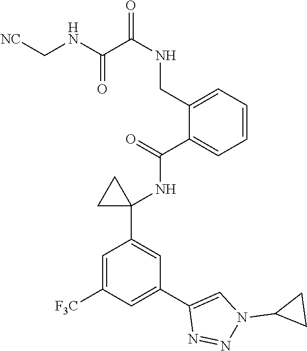 [SMILES: N#CCNC(=O)C(=O)NCc1ccccc1C(=O)NC1(c2cc(-c3cn(C4CC4)nn3)cc(C(F)(F)F)c2)CC1] | 552.24 |
| 61 | 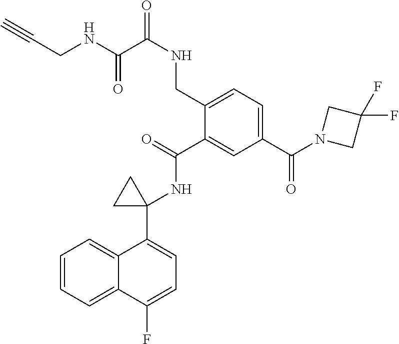 [SMILES: C#CCNC(=O)C(=O)NCc1ccc(C(=O)N2CC(F)(F)C2)cc1C(=O)NC1(c2ccc(F)c3ccccc23)CC1] | 563.22 |
| 62 | 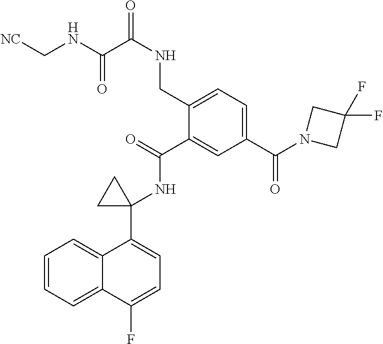 [SMILES: N#CCNC(=O)C(=O)NCc1ccc(C(=O)N2CC(F)(F)C2)cc1C(=O)NC1(c2ccc(F)c3ccccc23)CC1] | 564.14 |
| 63 | 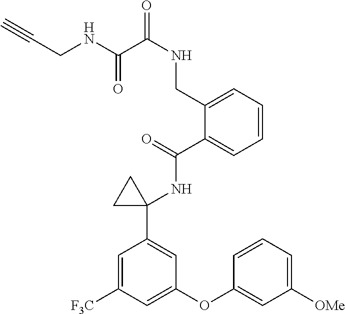 [SMILES: C#CCNC(=O)C(=O)NCc1ccccc1C(=O)NC1(c2cc(Oc3cccc(OC)c3)cc(C(F)(F)F)c2)CC1] | 566.30 |
| 64 | 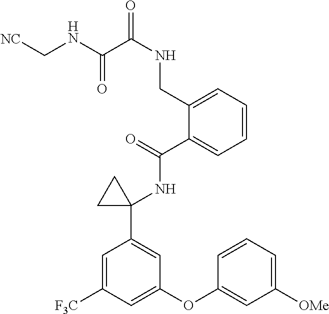 [SMILES: COc1cccc(Oc2cc(C(F)(F)F)cc(C3(NC(=O)c4ccccc4CNC(=O)C(=O)NCC#N)CC3)c2)c1] | 567.26 |
| 65 | 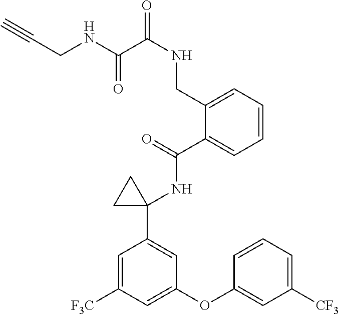 [SMILES: C#CCNC(=O)C(=O)NCc1ccccc1C(=O)NC1(c2cc(Oc3cccc(C(F)(F)F)c3)cc(C(F)(F)F)c2)CC1] | 604.27 |
| 66 | 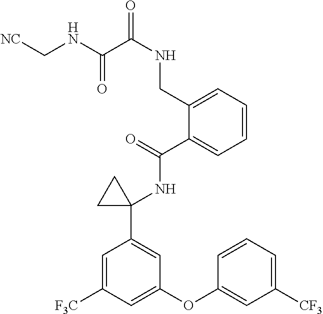 [SMILES: N#CCNC(=O)C(=O)NCc1ccccc1C(=O)NC1(c2cc(Oc3cccc(C(F)(F)F)c3)cc(C(F)(F)F)c2)CC1] | 605.23 |
| 67 | 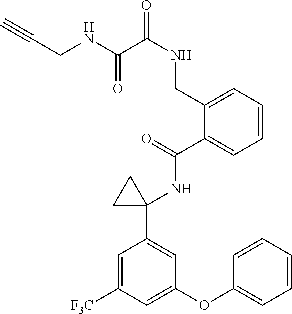 [SMILES: C#CCNC(=O)C(=O)NCc1ccccc1C(=O)NC1(c2cc(Oc3ccccc3)cc(C(F)(F)F)c2)CC1] | 536.25 |
| 68 | 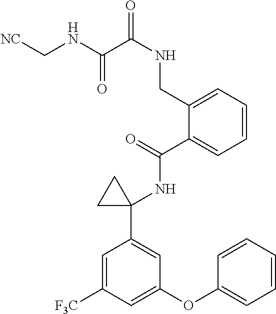 [SMILES: N#CCNC(=O)C(=O)NCc1ccccc1C(=O)NC1(c2cc(Oc3ccccc3)cc(C(F)(F)F)c2)CC1] | 537.31 |
| 69 | 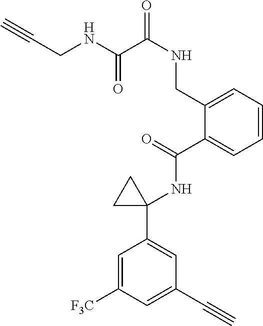 [SMILES: C#CCNC(=O)C(=O)NCc1ccccc1C(=O)NC1(c2cc(C#C)cc(C(F)(F)F)c2)CC1] | 468.14 |
| 70 | 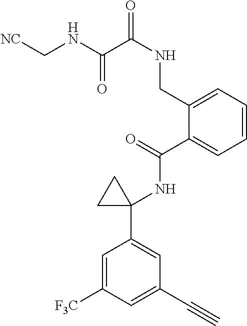 [SMILES: C#Cc1cc(C(F)(F)F)cc(C2(NC(=O)c3ccccc3CNC(=O)C(=O)NCC#N)CC2)c1] | 469.10 |
| 71 | 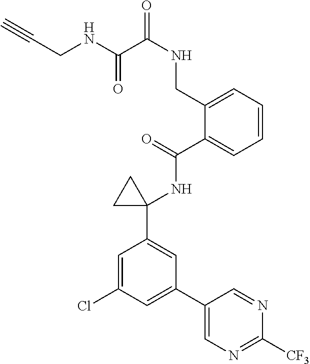 [SMILES: C#CCNC(=O)C(=O)NCc1ccccc1C(=O)NC1(c2cc(Cl)cc(-c3cnc(C(F)(F)F)nc3)c2)CC1] | 556.12 |
| 72 | 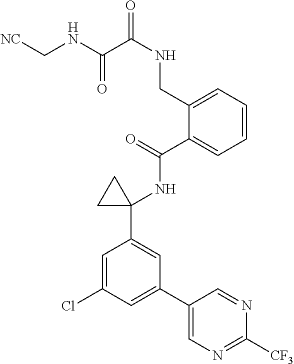 [SMILES: N#CCNC(=O)C(=O)NCc1ccccc1C(=O)NC1(c2cc(Cl)cc(-c3cnc(C(F)(F)F)nc3)c2)CC1] | 557.18 |
| 73 | 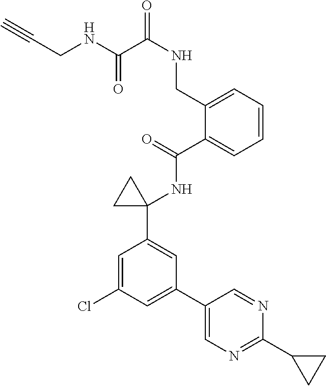 [SMILES: C#CCNC(=O)C(=O)NCc1ccccc1C(=O)NC1(c2cc(Cl)cc(-c3cnc(C4CC4)nc3)c2)CC1] | 528.28 |
| 74 | 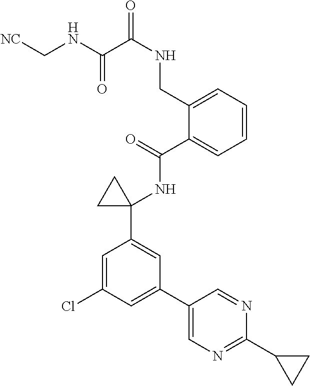 [SMILES: N#CCNC(=O)C(=O)NCc1ccccc1C(=O)NC1(c2cc(Cl)cc(-c3cnc(C4CC4)nc3)c2)CC1] | 529.29 |
| 75 | 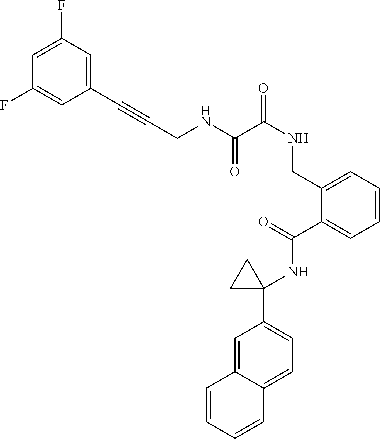 [SMILES: O=C(NCC#Cc1cc(F)cc(F)c1)C(=O)NCc1ccccc1C(=O)NC1(c2ccc3ccccc3c2)CC1] | 538.02 |
| 76 | 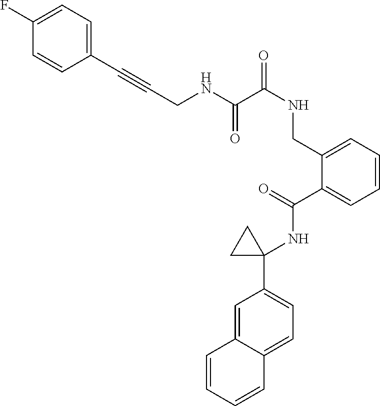 [SMILES: O=C(NCC#Cc1ccc(F)cc1)C(=O)NCc1ccccc1C(=O)NC1(c2ccc3ccccc3c2)CC1] | 520.06 |
| 77 | 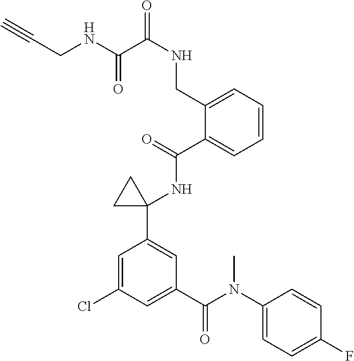 [SMILES: C#CCNC(=O)C(=O)NCc1ccccc1C(=O)NC1(c2cc(Cl)cc(C(=O)N(C)c3ccc(F)cc3)c2)CC1] | 561.16 |
| 78 | 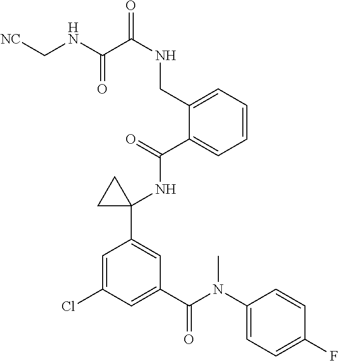 [SMILES: CN(C(=O)c1cc(Cl)cc(C2(NC(=O)c3ccccc3CNC(=O)C(=O)NCC#N)CC2)c1)c1ccc(F)cc1] | 562.22 |
| 79 | 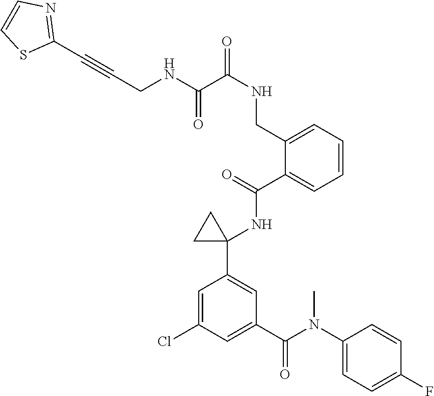 [SMILES: CN(C(=O)c1cc(Cl)cc(C2(NC(=O)c3ccccc3CNC(=O)C(=O)NCC#Cc3nccs3)CC2)c1)c1ccc(F)cc1] | 644.21 |
| 80 | 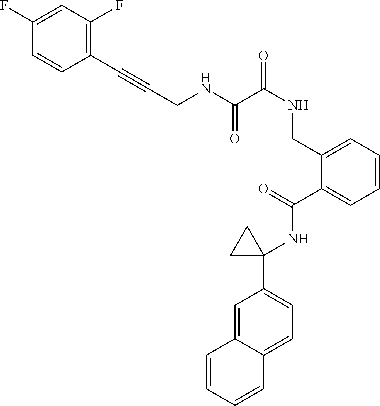 [SMILES: O=C(NCC#Cc1ccc(F)cc1F)C(=O)NCc1ccccc1C(=O)NC1(c2ccc3ccccc3c2)CC1] | 538.07 |
| 81 | 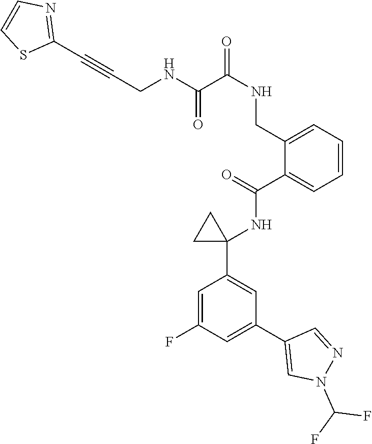 [SMILES: O=C(NCC#Cc1nccs1)C(=O)NCc1ccccc1C(=O)NC1(c2cc(F)cc(-c3cnn(C(F)F)c3)c2)CC1] | 593.07 |
| 82 | 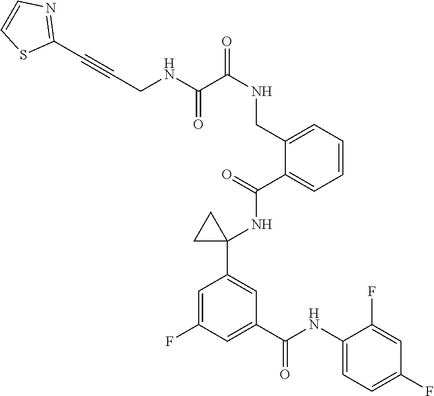 [SMILES: O=C(NCC#Cc1nccs1)C(=O)NCc1ccccc1C(=O)NC1(c2cc(F)cc(C(=O)Nc3ccc(F)cc3F)c2)CC1] | 632.05 |
| 83 | 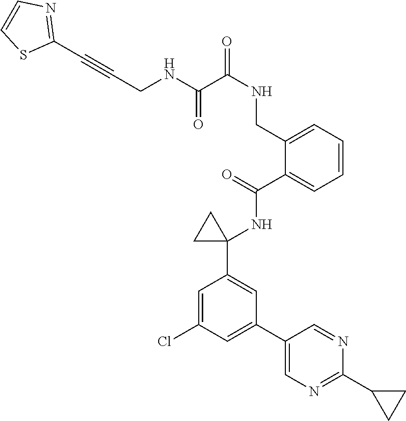 [SMILES: O=C(NCC#Cc1nccs1)C(=O)NCc1ccccc1C(=O)NC1(c2cc(Cl)cc(-c3cnc(C4CC4)nc3)c2)CC1] | 611.21 |
| 84 | 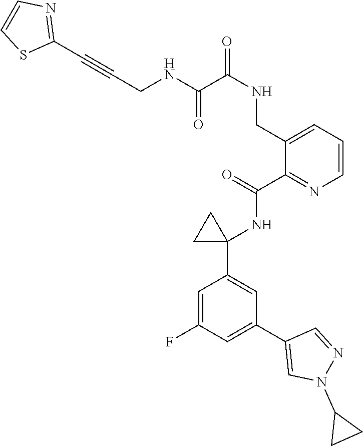 [SMILES: O=C(NCC#Cc1nccs1)C(=O)NCc1cccnc1C(=O)NC1(c2cc(F)cc(-c3cnn(C4CC4)c3)c2)CC1] | 584.19 |
| 85 | 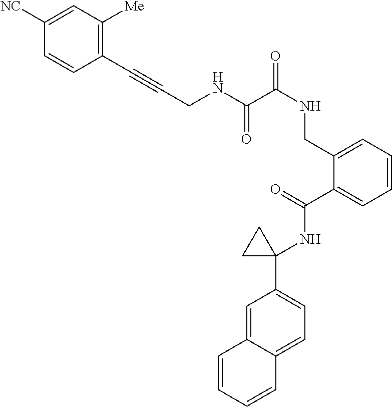 [SMILES: Cc1cc(C#N)ccc1C#CCNC(=O)C(=O)NCc1ccccc1C(=O)NC1(c2ccc3ccccc3c2)CC1] | 541.23 |
| 86 | 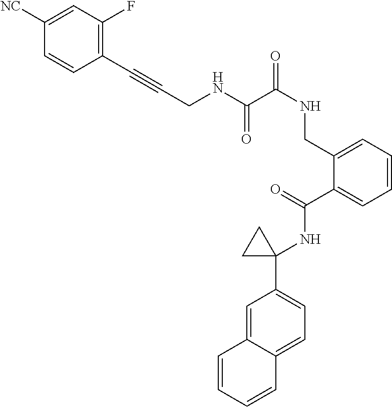 [SMILES: N#Cc1ccc(C#CCNC(=O)C(=O)NCc2ccccc2C(=O)NC2(c3ccc4ccccc4c3)CC2)c(F)c1] | 545.12 |
| 87 | 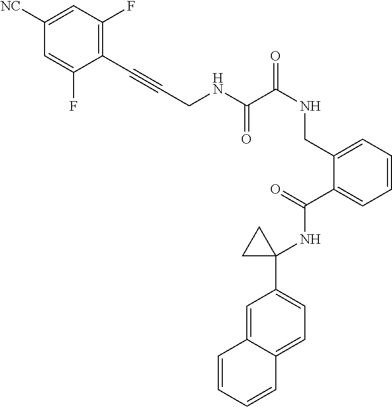 [SMILES: N#Cc1cc(F)c(C#CCNC(=O)C(=O)NCc2ccccc2C(=O)NC2(c3ccc4ccccc4c3)CC2)c(F)c1] | 563.02 |
| 88 | 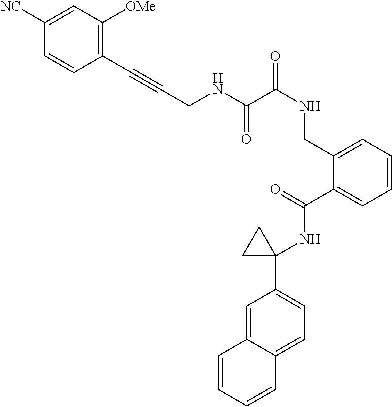 [SMILES: COc1cc(C#N)ccc1C#CCNC(=O)C(=O)NCc1ccccc1C(=O)NC1(c2ccc3ccccc3c2)CC1] | 557.07 |
| 89 | 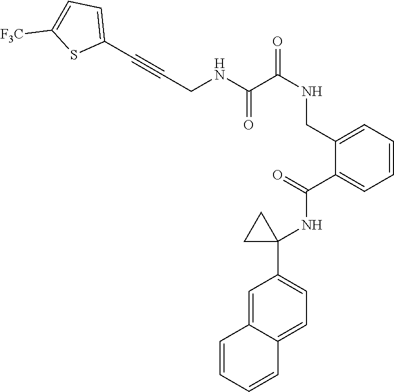 [SMILES: O=C(NCC#Cc1ccc(C(F)(F)F)s1)C(=O)NCc1ccccc1C(=O)NC1(c2ccc3ccccc3c2)CC1] | 576.03 |
| 90 | 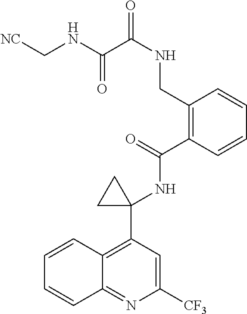 [SMILES: N#CCNC(=O)C(=O)NCc1ccccc1C(=O)NC1(c2cc(C(F)(F)F)nc3ccccc23)CC1] | 496.16 |
| 91 | 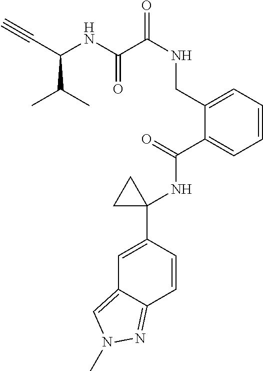 [SMILES: C#C[C@@H](NC(=O)C(=O)NCc1ccccc1C(=O)NC1(c2ccc3nn(C)cc3c2)CC1)C(C)C] | 472.31 |
| 92 | 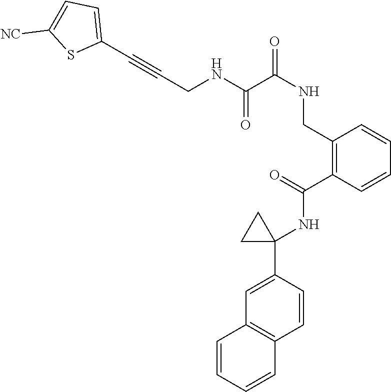 [SMILES: N#Cc1ccc(C#CCNC(=O)C(=O)NCc2ccccc2C(=O)NC2(c3ccc4ccccc4c3)CC2)s1] | 533.12 |
| 93 | 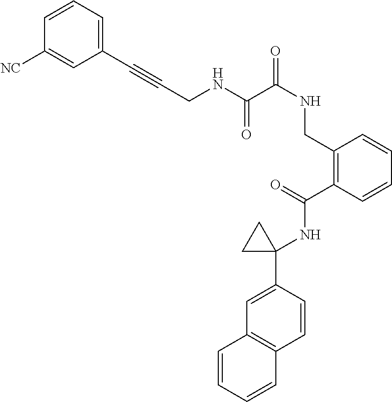 [SMILES: N#Cc1cccc(C#CCNC(=O)C(=O)NCc2ccccc2C(=O)NC2(c3ccc4ccccc4c3)CC2)c1] | 527.17 |
| 94 | 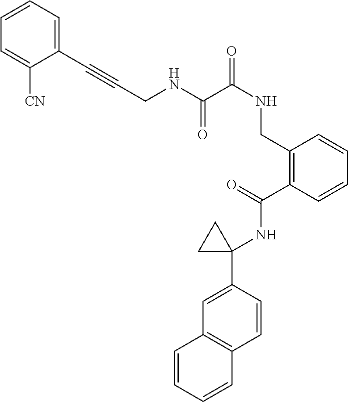 [SMILES: N#Cc1ccccc1C#CCNC(=O)C(=O)NCc1ccccc1C(=O)NC1(c2ccc3ccccc3c2)CC1] | 527.02 |
| 95 | 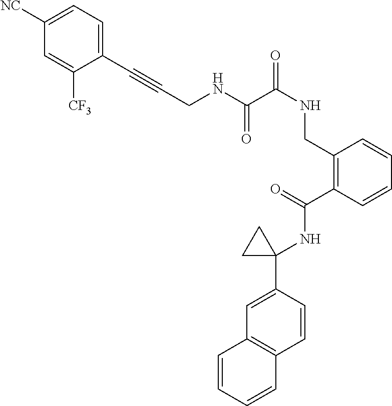 [SMILES: N#Cc1ccc(C#CCNC(=O)C(=O)NCc2ccccc2C(=O)NC2(c3ccc4ccccc4c3)CC2)c(C(F)(F)F)c1] | 595.09 |
| 96 | 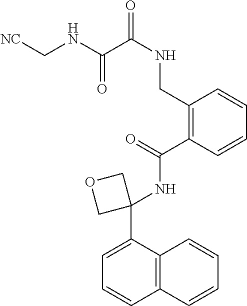 [SMILES: N#CCNC(=O)C(=O)NCc1ccccc1C(=O)NC1(c2cccc3ccccc23)COC1] | 443.27 |
| 97 | 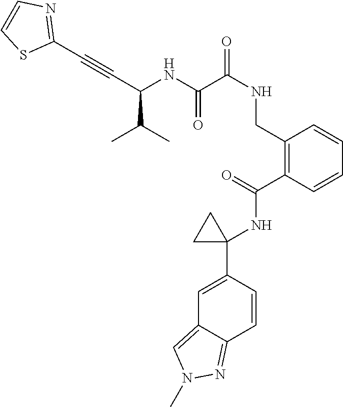 [SMILES: CC(C)[C@@H](C#Cc1nccs1)NC(=O)C(=O)NCc1ccccc1C(=O)NC1(c2ccc3nn(C)cc3c2)CC1] | 555.15 |
| 98 | 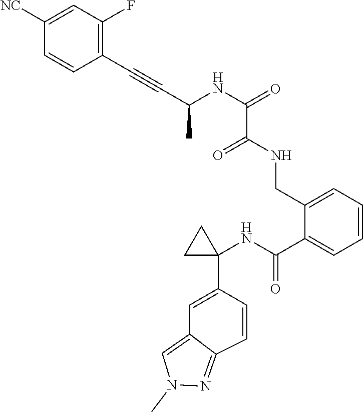 [SMILES: C[C@@H](C#Cc1ccc(C#N)cc1F)NC(=O)C(=O)NCc1ccccc1C(=O)NC1(c2ccc3nn(C)cc3c2)CC1] | 563.21 |
| 99 | 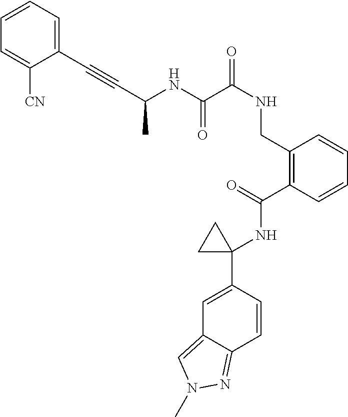 [SMILES: C[C@@H](C#Cc1ccccc1C#N)NC(=O)C(=O)NCc1ccccc1C(=O)NC1(c2ccc3nn(C)cc3c2)CC1] | 545.21 |
| 100 | 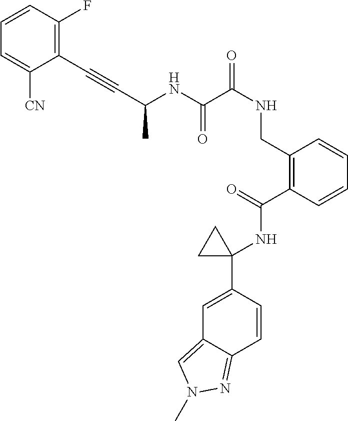 [SMILES: C[C@@H](C#Cc1c(F)cccc1C#N)NC(=O)C(=O)NCc1ccccc1C(=O)NC1(c2ccc3nn(C)cc3c2)CC1] | 563.21 |
| 101 | 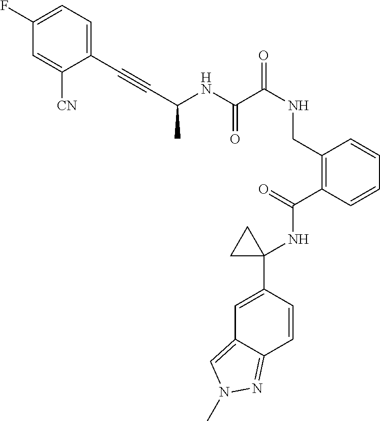 [SMILES: C[C@@H](C#Cc1ccc(F)cc1C#N)NC(=O)C(=O)NCc1ccccc1C(=O)NC1(c2ccc3nn(C)cc3c2)CC1] | 563.21 |
| 102 | 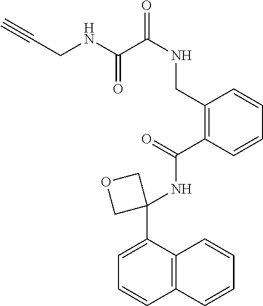 [SMILES: C#CCNC(=O)C(=O)NCc1ccccc1C(=O)NC1(c2cccc3ccccc23)COC1] | 442.26 |
| 103 | 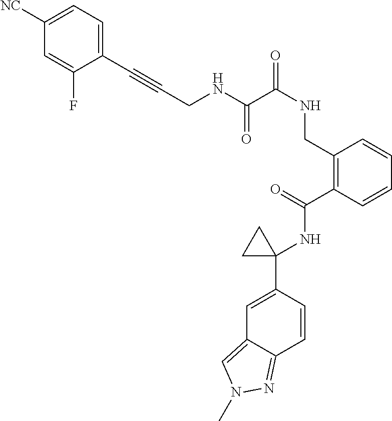 [SMILES: Cn1cc2cc(C3(NC(=O)c4ccccc4CNC(=O)C(=O)NCC#Cc4ccc(C#N)cc4F)CC3)ccc2n1] | 549.25 |
| 104 | 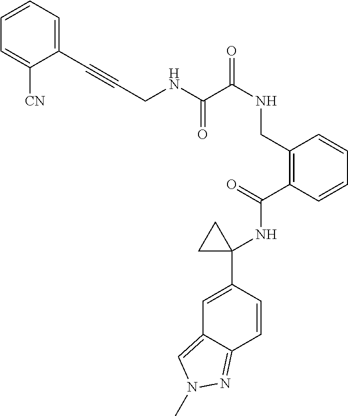 [SMILES: Cn1cc2cc(C3(NC(=O)c4ccccc4CNC(=O)C(=O)NCC#Cc4ccccc4C#N)CC3)ccc2n1] | 531.10 |
| 105 | 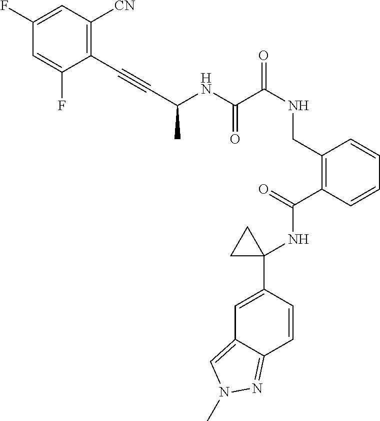 [SMILES: C[C@@H](C#Cc1c(F)cc(F)cc1C#N)NC(=O)C(=O)NCc1ccccc1C(=O)NC1(c2ccc3nn(C)cc3c2)CC1] | 581.21 |
| 106 | 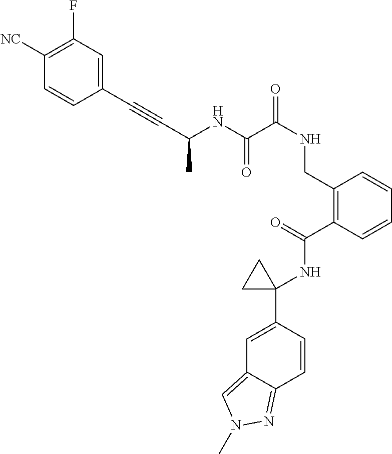 [SMILES: C[C@@H](C#Cc1ccc(C#N)c(F)c1)NC(=O)C(=O)NCc1ccccc1C(=O)NC1(c2ccc3nn(C)cc3c2)CC1] | 563.17 |
| 107 | 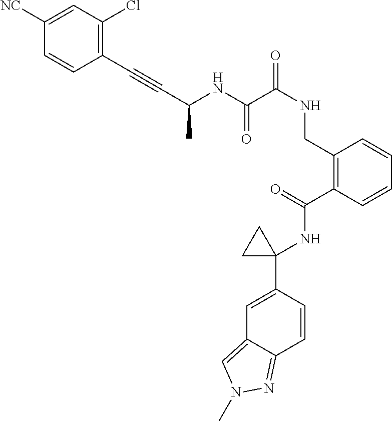 [SMILES: C[C@@H](C#Cc1ccc(C#N)cc1Cl)NC(=O)C(=O)NCc1ccccc1C(=O)NC1(c2ccc3nn(C)cc3c2)CC1] | 579.10 |
| 108 | 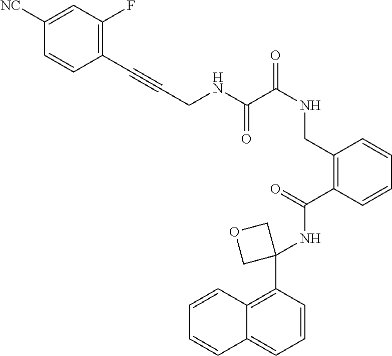 [SMILES: N#Cc1ccc(C#CCNC(=O)C(=O)NCc2ccccc2C(=O)NC2(c3cccc4ccccc34)COC2)c(F)c1] | 561.21 |
| 109 | 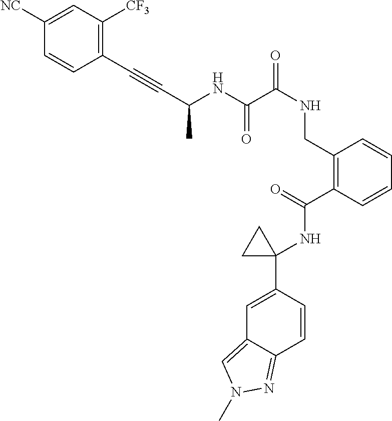 [SMILES: C[C@@H](C#Cc1ccc(C#N)cc1C(F)(F)F)NC(=O)C(=O)NCc1ccccc1C(=O)NC1(c2ccc3nn(C)cc3c2)CC1] | 613.13 |
| 110 | 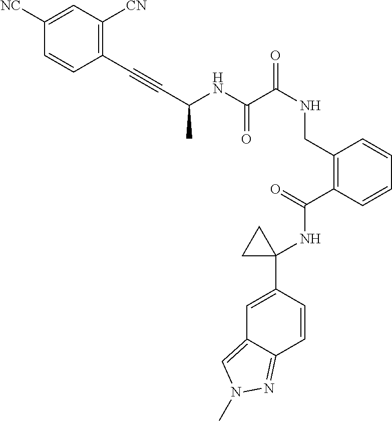 [SMILES: C[C@@H](C#Cc1ccc(C#N)cc1C#N)NC(=O)C(=O)NCc1ccccc1C(=O)NC1(c2ccc3nn(C)cc3c2)CC1] | 570.08 |
| 111 | 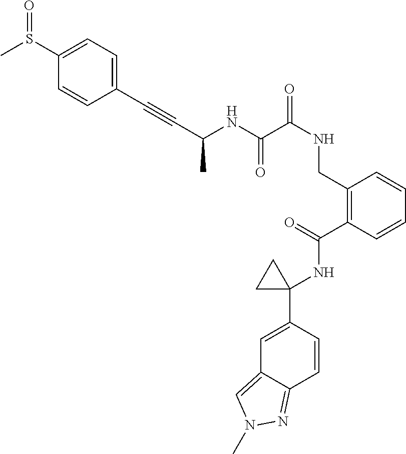 [SMILES: C[C@@H](C#Cc1ccc(S(C)=O)cc1)NC(=O)C(=O)NCc1ccccc1C(=O)NC1(c2ccc3nn(C)cc3c2)CC1] | 582.13 |
| 112 | 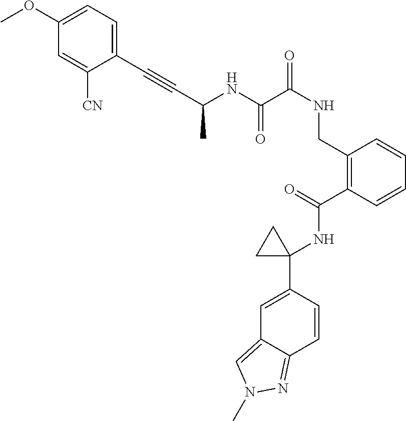 [SMILES: COc1ccc(C#C[C@H](C)NC(=O)C(=O)NCc2ccccc2C(=O)NC2(c3ccc4nn(C)cc4c3)CC2)c(C#N)c1] | 575.21 |
| 113 | 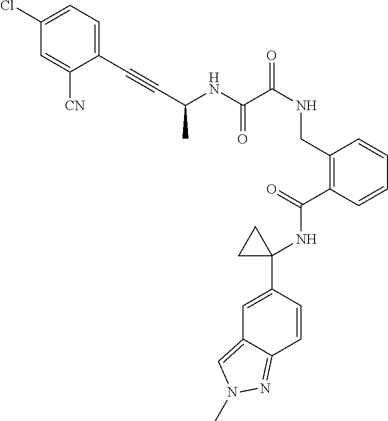 [SMILES: C[C@@H](C#Cc1ccc(Cl)cc1C#N)NC(=O)C(=O)NCc1ccccc1C(=O)NC1(c2ccc3nn(C)cc3c2)CC1] | 579.15 |
| 114 | 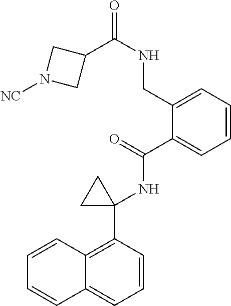 [SMILES: N#CN1CC(C(=O)NCc2ccccc2C(=O)NC2(c3cccc4ccccc34)CC2)C1] | 425.28 |
| 115 | 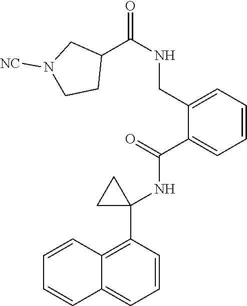 [SMILES: N#CN1CCC(C(=O)NCc2ccccc2C(=O)NC2(c3cccc4ccccc34)CC2)C1] | 439.29 |
| 116 | 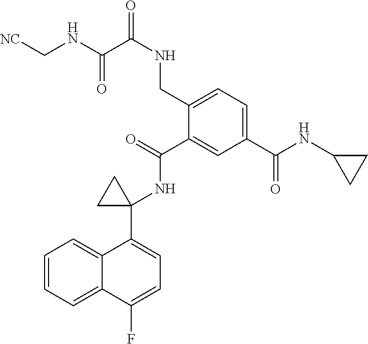 [SMILES: N#CCNC(=O)C(=O)NCc1ccc(C(=O)NC2CC2)cc1C(=O)NC1(c2ccc(F)c3ccccc23)CC1] | 528.03 |
| 117 | 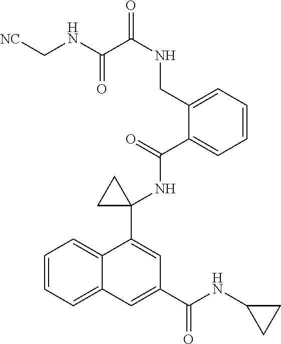 [SMILES: N#CCNC(=O)C(=O)NCc1ccccc1C(=O)NC1(c2cc(C(=O)NC3CC3)cc3ccccc23)CC1] | 510.08 |
| 118 | 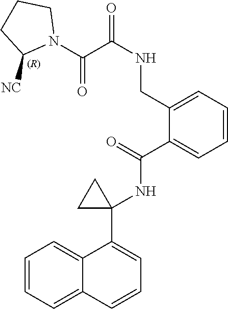 | 467.32 |
| 119 | 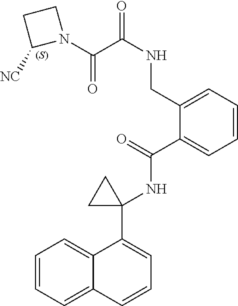 | 453.29 |
| 120 | 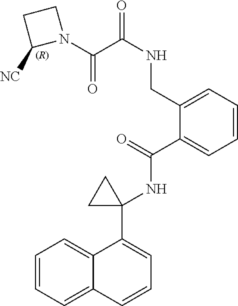 | 453.29 |
| 121 | 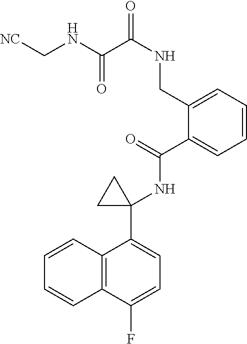 [SMILES: N#CCNC(=O)C(=O)NCc1ccccc1C(=O)NC1(c2ccc(F)c3ccccc23)CC1] | 445.20 |
| 122 | 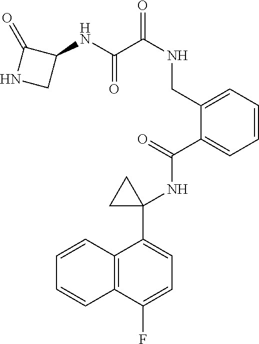 [SMILES: O=C(NCc1ccccc1C(=O)NC1(c2ccc(F)c3ccccc23)CC1)C(=O)N[C@H]1CNC1=O] | 475.19 |
| 123 | 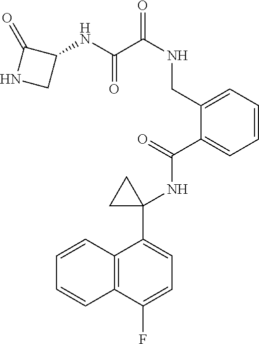 [SMILES: O=C(NCc1ccccc1C(=O)NC1(c2ccc(F)c3ccccc23)CC1)C(=O)N[C@@H]1CNC1=O] | 475.24 |
| 124 | 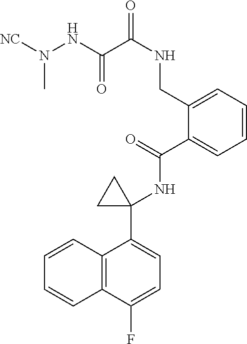 [SMILES: CN(C#N)NC(=O)C(=O)NCc1ccccc1C(=O)NC1(c2ccc(F)c3ccccc23)CC1] | 460.34 |
| 125 | 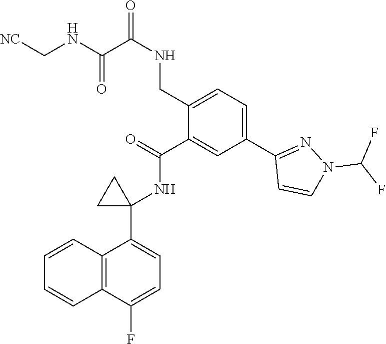 [SMILES: N#CCNC(=O)C(=O)NCc1ccc(-c2ccn(C(F)F)n2)cc1C(=O)NC1(c2ccc(F)c3ccccc23)CC1] | 561.24 |
| 126 | 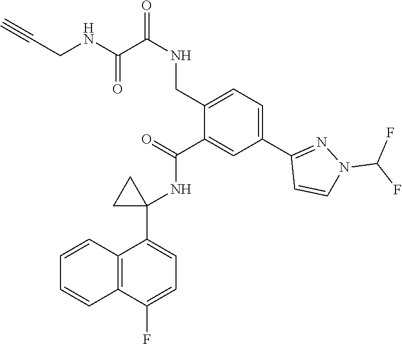 [SMILES: C#CCNC(=O)C(=O)NCc1ccc(-c2ccn(C(F)F)n2)cc1C(=O)NC1(c2ccc(F)c3ccccc23)CC1] | 560.25 |
| 127 | 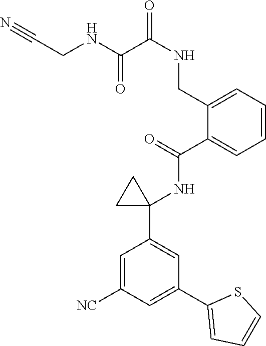 [SMILES: N#CCNC(=O)C(=O)NCc1ccccc1C(=O)NC1(c2cc(C#N)cc(-c3cccs3)c2)CC1] | 484.10 |
| 128 | 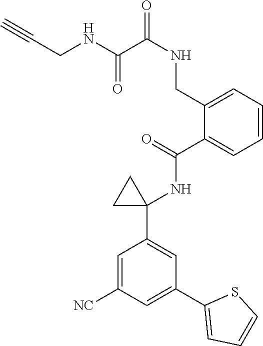 [SMILES: C#CCNC(=O)C(=O)NCc1ccccc1C(=O)NC1(c2cc(C#N)cc(-c3cccs3)c2)CC1] | 483.19 |
| 129 | 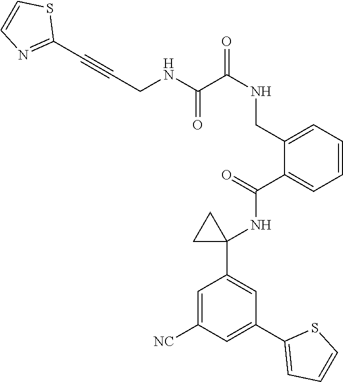 [SMILES: N#Cc1cc(-c2cccs2)cc(C2(NC(=O)c3ccccc3CNC(=O)C(=O)NCC#Cc3nccs3)CC2)c1] | 566.19 |
| 130 | 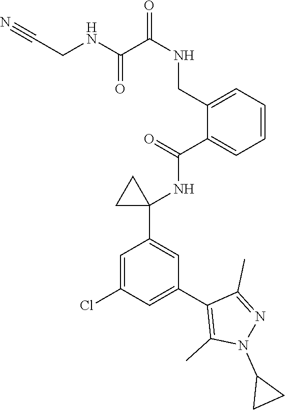 [SMILES: Cc1nn(C2CC2)c(C)c1-c1cc(Cl)cc(C2(NC(=O)c3ccccc3CNC(=O)C(=O)NCC#N)CC2)c1] | 545.31 |
| 131 | 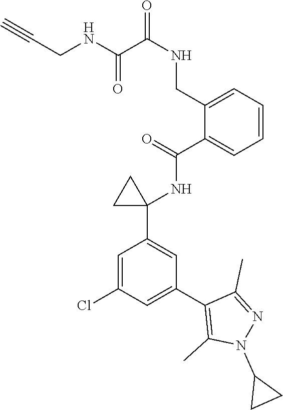 [SMILES: C#CCNC(=O)C(=O)NCc1ccccc1C(=O)NC1(c2cc(Cl)cc(-c3c(C)nn(C4CC4)c3C)c2)CC1] | 544.30 |
| 132 | 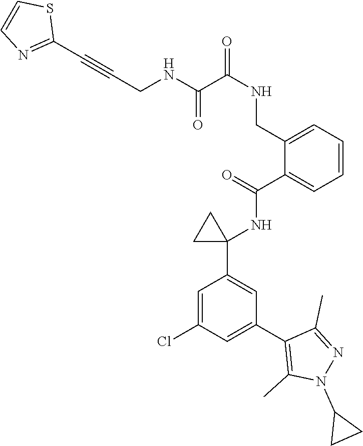 [SMILES: Cc1nn(C2CC2)c(C)c1-c1cc(Cl)cc(C2(NC(=O)c3ccccc3CNC(=O)C(=O)NCC#Cc3nccs3)CC2)c1] | 627.29 |
| 133 | 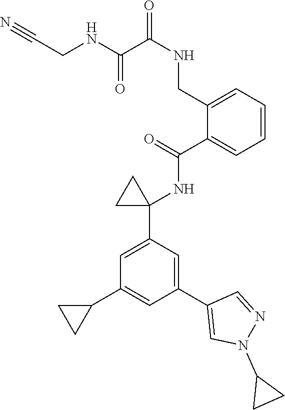 [SMILES: N#CCNC(=O)C(=O)NCc1ccccc1C(=O)NC1(c2cc(-c3cnn(C4CC4)c3)cc(C3CC3)c2)CC1] | 523.30 |
| 134 | 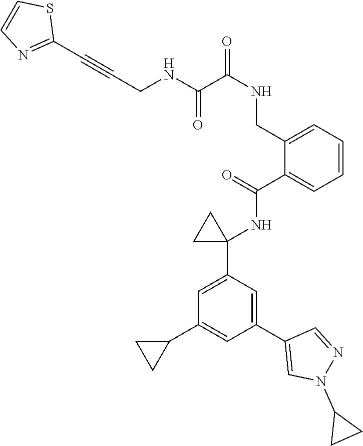 [SMILES: O=C(NCC#Cc1nccs1)C(=O)NCc1ccccc1C(=O)NC1(c2cc(-c3cnn(C4CC4)c3)cc(C3CC3)c2)CC1] | 605.28 |
| 135 | 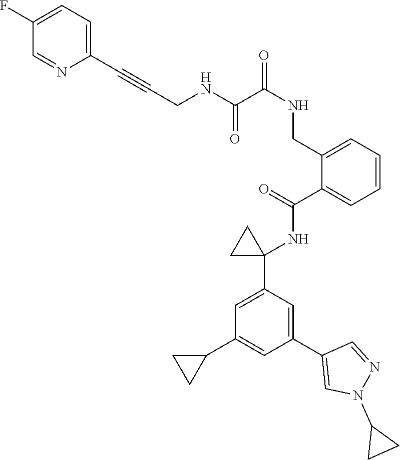 [SMILES: O=C(NCC#Cc1ccc(F)cn1)C(=O)NCc1ccccc1C(=O)NC1(c2cc(-c3cnn(C4CC4)c3)cc(C3CC3)c2)CC1] | 617.32 |
| 136 | 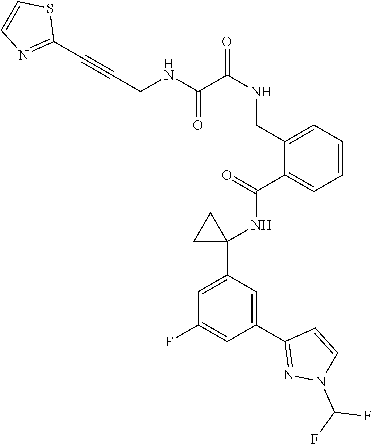 [SMILES: O=C(NCC#Cc1nccs1)C(=O)NCc1ccccc1C(=O)NC1(c2cc(F)cc(-c3ccn(C(F)F)n3)c2)CC1] | 593.16 |
| 137 | 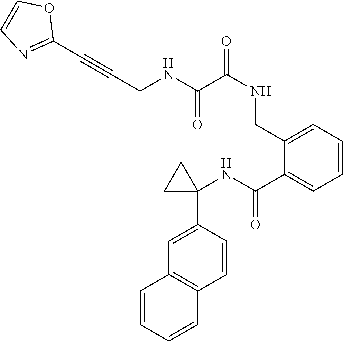 [SMILES: O=C(NCC#Cc1ncco1)C(=O)NCc1ccccc1C(=O)NC1(c2ccc3ccccc3c2)CC1] | 493.19 |
| 138 | 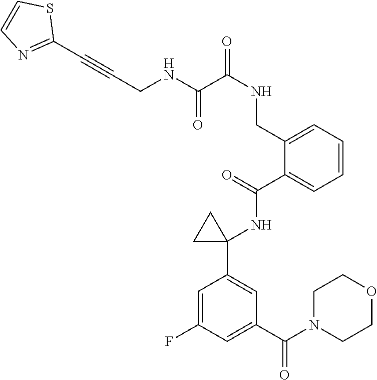 [SMILES: O=C(NCC#Cc1nccs1)C(=O)NCc1ccccc1C(=O)NC1(c2cc(F)cc(C(=O)N3CCOCC3)c2)CC1] | 590.14 |
| 139 | 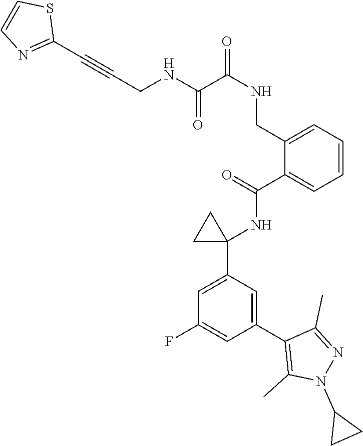 [SMILES: Cc1nn(C2CC2)c(C)c1-c1cc(F)cc(C2(NC(=O)c3ccccc3CNC(=O)C(=O)NCC#Cc3nccs3)CC2)c1] | 611.24 |
| 140 | 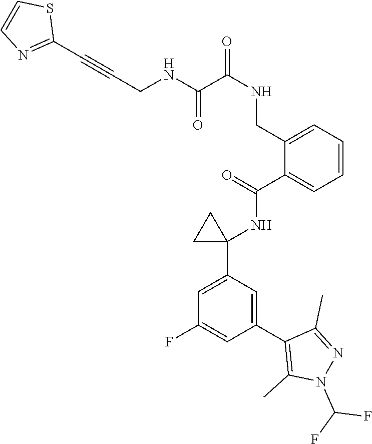 [SMILES: Cc1nn(C(F)F)c(C)c1-c1cc(F)cc(C2(NC(=O)c3ccccc3CNC(=O)C(=O)NCC#Cc3nccs3)CC2)c1] | 621.17 |
| 141 | 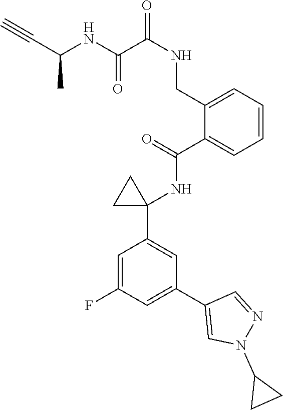 [SMILES: C#C[C@H](C)NC(=O)C(=O)NCc1ccccc1C(=O)NC1(c2cc(F)cc(-c3cnn(C4CC4)c3)c2)CC1] | 514.25 |
| 142 | 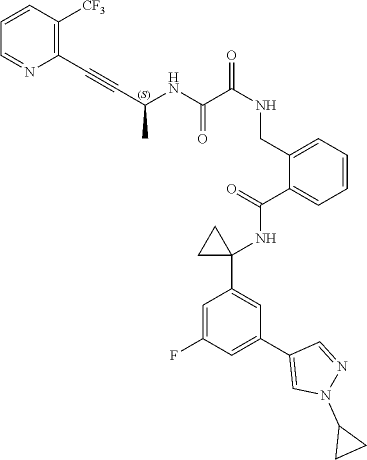 | 659.26 |
| 143 | 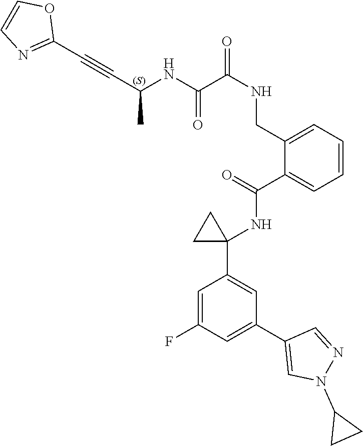 | 581.38 |
| 144 | 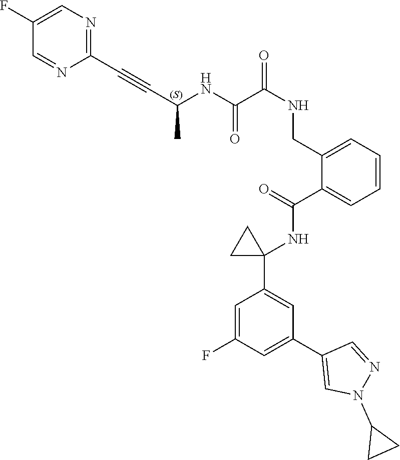 | 610.33 |
| 145 | 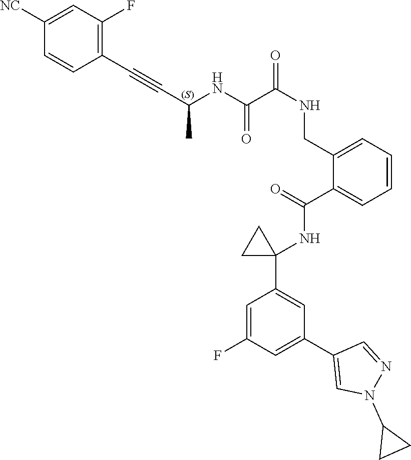 | 633.27 |
| 146 | 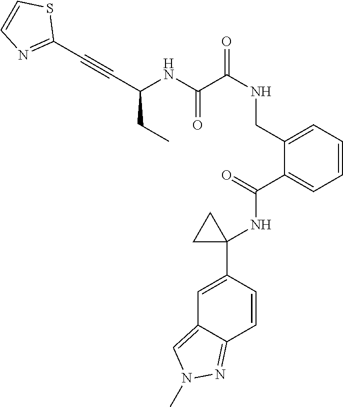 [SMILES: CC[C@@H](C#Cc1nccs1)NC(=O)C(=O)NCc1ccccc1C(=O)NC1(c2ccc3nn(C)cc3c2)CC1] | 541.33 |
| 147 | 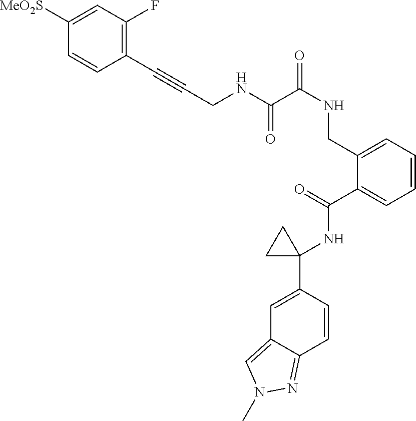 [SMILES: Cn1cc2cc(C3(NC(=O)c4ccccc4CNC(=O)C(=O)NCC#Cc4ccc(S(C)(=O)=O)cc4F)CC3)ccc2n1] | 602.40 |
| 148 | 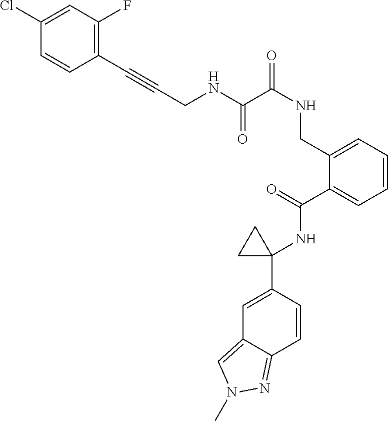 [SMILES: Cn1cc2cc(C3(NC(=O)c4ccccc4CNC(=O)C(=O)NCC#Cc4ccc(Cl)cc4F)CC3)ccc2n1] | 558.31 |
| 149 | 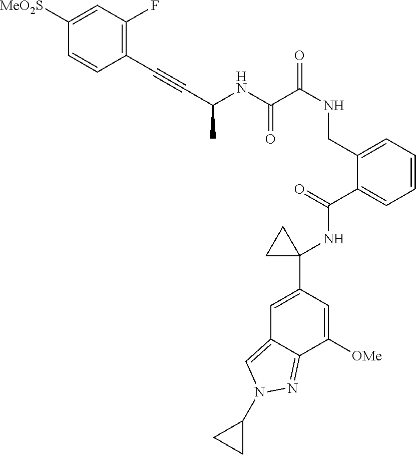 [SMILES: COc1cc(C2(NC(=O)c3ccccc3CNC(=O)C(=O)N[C@@H](C)C#Cc3ccc(S(C)(=O)=O)cc3F)CC2)cc2cn(C3CC3)nc12] | 672.28 |
| 150 | 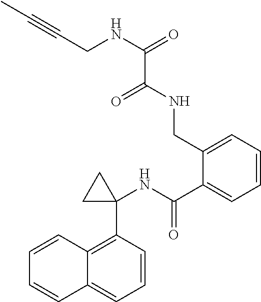 [SMILES: CC#CCNC(=O)C(=O)NCc1ccccc1C(=O)NC1(c2cccc3ccccc23)CC1] | 440.35 |
| 151 | 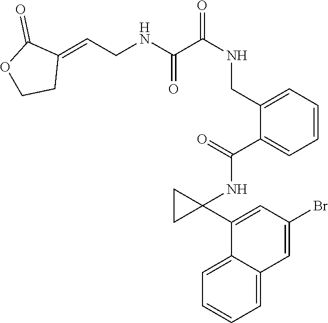 [SMILES: O=C(NC/C=C1\CCOC1=O)C(=O)NCc1ccccc1C(=O)NC1(c2cc(Br)cc3ccccc23)CC1] | 576.14 |
| 152 | 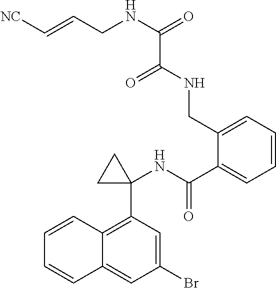 [SMILES: N#C/C=C/CNC(=O)C(=O)NCc1ccccc1C(=O)NC1(c2cc(Br)cc3ccccc23)CC1] | 531.21 |
| 153 | 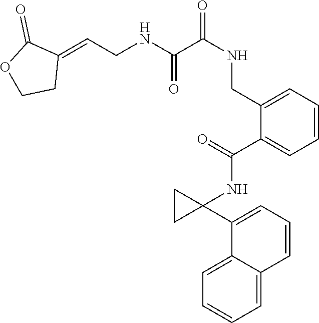 [SMILES: O=C(NC/C=C1\CCOC1=O)C(=O)NCc1ccccc1C(=O)NC1(c2cccc3ccccc23)CC1] | 498.38 |
| 154 | 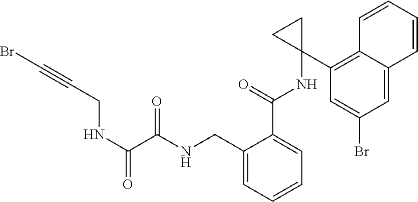 [SMILES: O=C(NCC#CBr)C(=O)NCc1ccccc1C(=O)NC1(c2cc(Br)cc3ccccc23)CC1] | 581.90 |
| 155 | 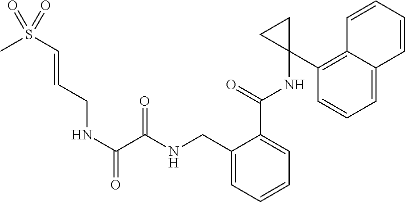 [SMILES: CS(=O)(=O)/C=C/CNC(=O)C(=O)NCc1ccccc1C(=O)NC1(c2cccc3ccccc23)CC1] | 506.11 |
| 156 | 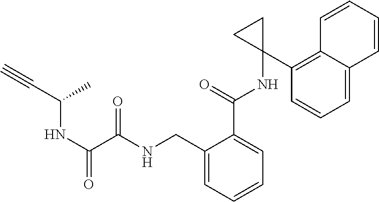 [SMILES: C#C[C@H](C)NC(=O)C(=O)NCc1ccccc1C(=O)NC1(c2cccc3ccccc23)CC1] | 440.25 |
| 157 | 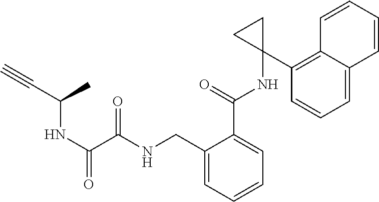 [SMILES: C#C[C@@H](C)NC(=O)C(=O)NCc1ccccc1C(=O)NC1(c2cccc3ccccc23)CC1] | 440.30 |
| 158 | 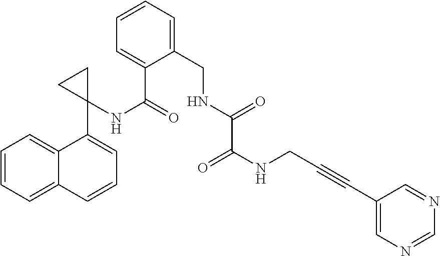 [SMILES: O=C(NCC#Cc1cncnc1)C(=O)NCc1ccccc1C(=O)NC1(c2cccc3ccccc23)CC1] | 504.28 |
| 159 | 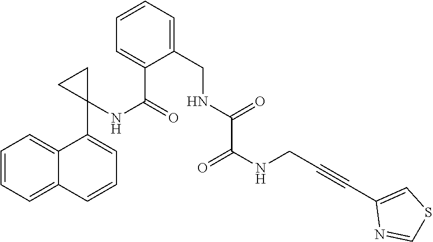 [SMILES: O=C(NCC#Cc1cscn1)C(=O)NCc1ccccc1C(=O)NC1(c2cccc3ccccc23)CC1] | 509.18 |
| 160 | 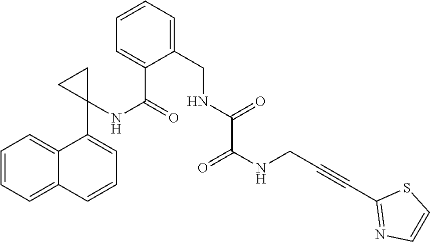 [SMILES: O=C(NCC#Cc1nccs1)C(=O)NCc1ccccc1C(=O)NC1(c2cccc3ccccc23)CC1] | 509.23 |
| 161 | 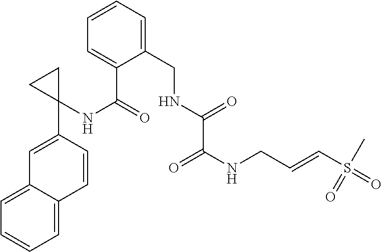 [SMILES: CS(=O)(=O)/C=C/CNC(=O)C(=O)NCc1ccccc1C(=O)NC1(c2ccc3ccccc3c2)CC1] | 506.25 |
| 162 | 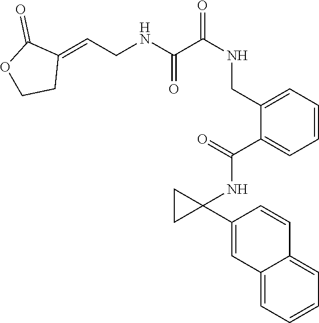 [SMILES: O=C(NC/C=C1\CCOC1=O)C(=O)NCc1ccccc1C(=O)NC1(c2ccc3ccccc3c2)CC1] | 498.23 |
| 163 | 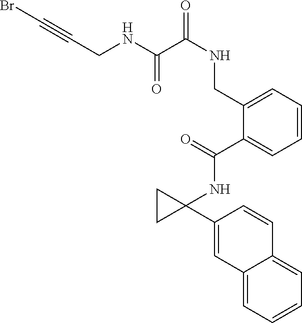 [SMILES: O=C(NCC#CBr)C(=O)NCc1ccccc1C(=O)NC1(c2ccc3ccccc3c2)CC1] | 504.09 |
| 164 | 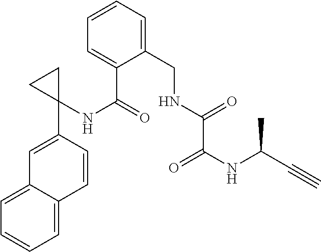 [SMILES: C#C[C@H](C)NC(=O)C(=O)NCc1ccccc1C(=O)NC1(c2ccc3ccccc3c2)CC1] | 440.20 |
| 165 | 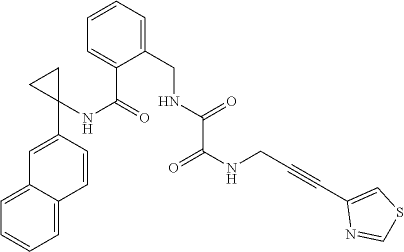 [SMILES: O=C(NCC#Cc1cscn1)C(=O)NCc1ccccc1C(=O)NC1(c2ccc3ccccc3c2)CC1] | 509.23 |
| 166 | 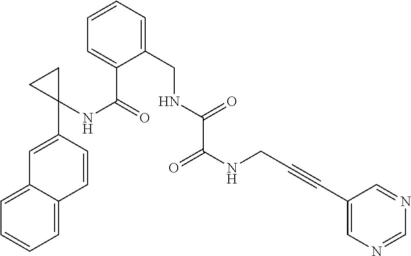 [SMILES: O=C(NCC#Cc1cncnc1)C(=O)NCc1ccccc1C(=O)NC1(c2ccc3ccccc3c2)CC1] | 504.19 |
| 167 | 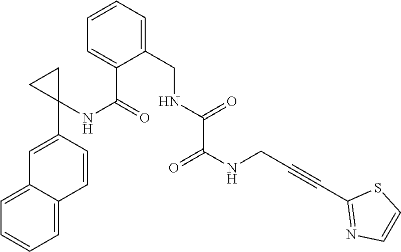 [SMILES: O=C(NCC#Cc1nccs1)C(=O)NCc1ccccc1C(=O)NC1(c2ccc3ccccc3c2)CC1] | 509.23 |
| 168 | 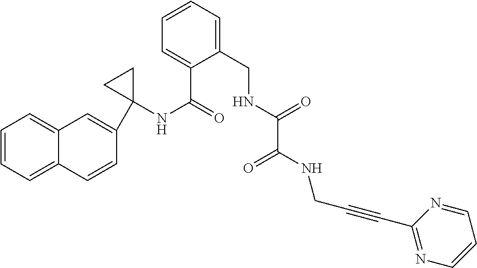 [SMILES: O=C(NCC#Cc1ncccn1)C(=O)NCc1ccccc1C(=O)NC1(c2ccc3ccccc3c2)CC1] | 504.19 |
| 169 | 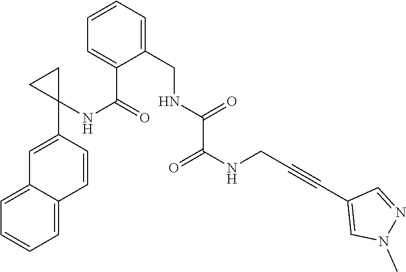 [SMILES: Cn1cc(C#CCNC(=O)C(=O)NCc2ccccc2C(=O)NC2(c3ccc4ccccc4c3)CC2)cn1] | 506.30 |
| 170 | 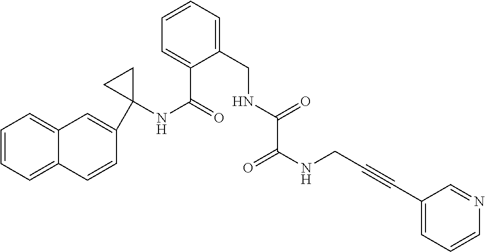 [SMILES: O=C(NCC#Cc1cccnc1)C(=O)NCc1ccccc1C(=O)NC1(c2ccc3ccccc3c2)CC1] | 503.28 |
| 171 | 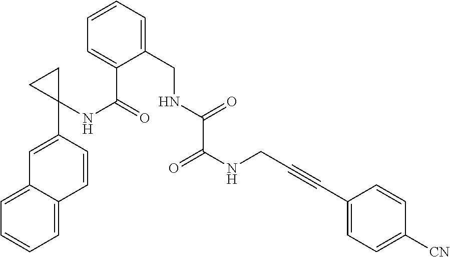 [SMILES: N#Cc1ccc(C#CCNC(=O)C(=O)NCc2ccccc2C(=O)NC2(c3ccc4ccccc4c3)CC2)cc1] | 527.13 |
| 172 | 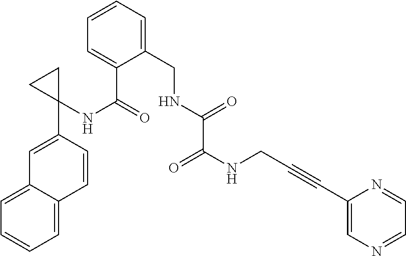 [SMILES: O=C(NCC#Cc1cnccn1)C(=O)NCc1ccccc1C(=O)NC1(c2ccc3ccccc3c2)CC1] | 504.19 |
| 173 | 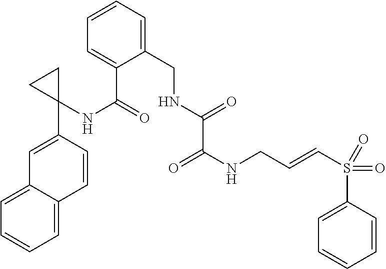 [SMILES: O=C(NC/C=C/S(=O)(=O)c1ccccc1)C(=O)NCc1ccccc1C(=O)NC1(c2ccc3ccccc3c2)CC1] | 568.22 |
| 174 | 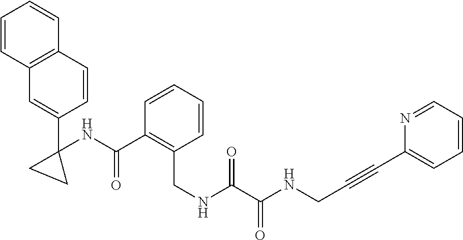 [SMILES: O=C(NCC#Cc1ccccn1)C(=O)NCc1ccccc1C(=O)NC1(c2ccc3ccccc3c2)CC1] | 503.37 |
| 175 | 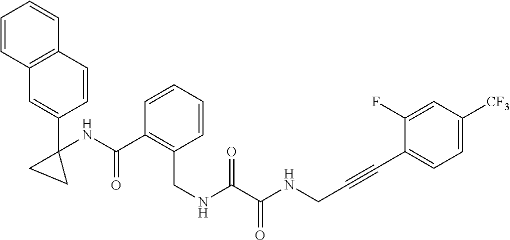 [SMILES: O=C(NCC#Cc1ccc(C(F)(F)F)cc1F)C(=O)NCc1ccccc1C(=O)NC1(c2ccc3ccccc3c2)CC1] | 588.24 |
| 176 | 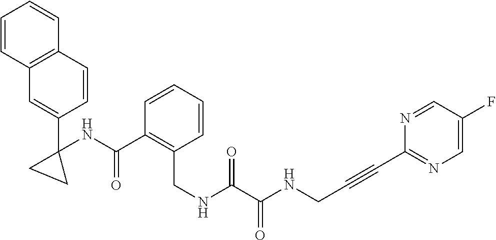 [SMILES: O=C(NCC#Cc1ncc(F)cn1)C(=O)NCc1ccccc1C(=O)NC1(c2ccc3ccccc3c2)CC1] | 522.09 |
| 177 | 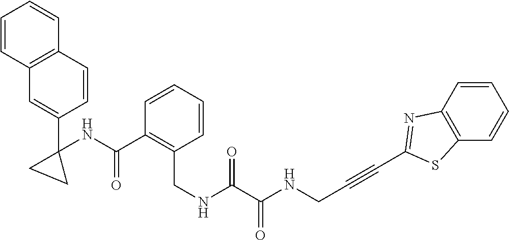 [SMILES: O=C(NCC#Cc1nc2ccccc2s1)C(=O)NCc1ccccc1C(=O)NC1(c2ccc3ccccc3c2)CC1] | 559.20 |
| 178 | 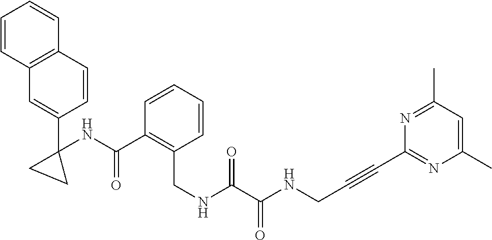 [SMILES: Cc1cc(C)nc(C#CCNC(=O)C(=O)NCc2ccccc2C(=O)NC2(c3ccc4ccccc4c3)CC2)n1] | 532.32 |
| 179 | 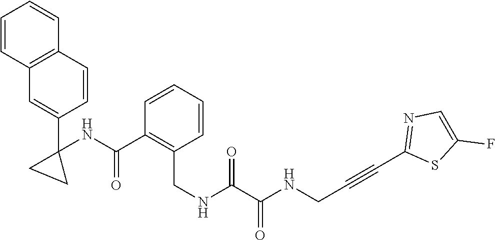 [SMILES: O=C(NCC#Cc1ncc(F)s1)C(=O)NCc1ccccc1C(=O)NC1(c2ccc3ccccc3c2)CC1] | 527.18 |
| 180 | 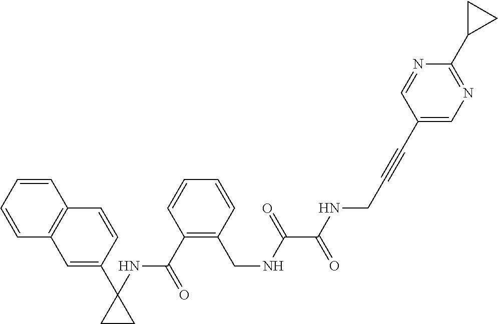 [SMILES: O=C(NCC#Cc1cnc(C2CC2)nc1)C(=O)NCc1ccccc1C(=O)NC1(c2ccc3ccccc3c2)CC1] | 544.32 |
| 181 | 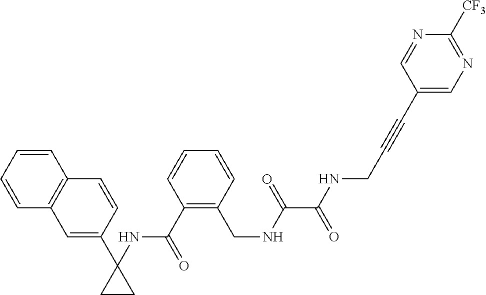 [SMILES: O=C(NCC#Cc1cnc(C(F)(F)F)nc1)C(=O)NCc1ccccc1C(=O)NC1(c2ccc3ccccc3c2)CC1] | 572.20 |
| 182 | 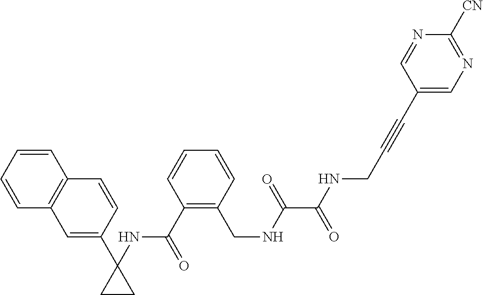 [SMILES: N#Cc1ncc(C#CCNC(=O)C(=O)NCc2ccccc2C(=O)NC2(c3ccc4ccccc4c3)CC2)cn1] | 529.15 |
| 183 | 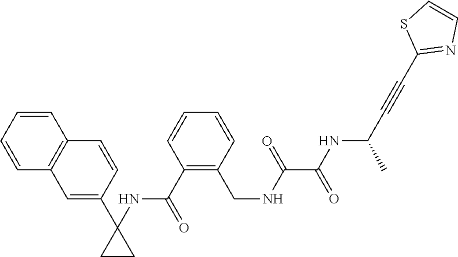 [SMILES: C[C@@H](C#Cc1nccs1)NC(=O)C(=O)NCc1ccccc1C(=O)NC1(c2ccc3ccccc3c2)CC1] | 523.15 |
| 184 | 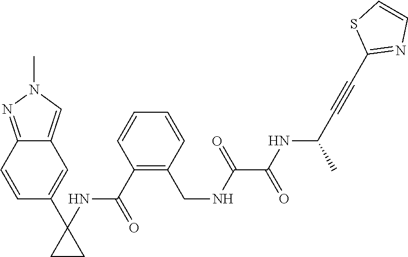 [SMILES: C[C@@H](C#Cc1nccs1)NC(=O)C(=O)NCc1ccccc1C(=O)NC1(c2ccc3nn(C)cc3c2)CC1] | 527.32 |
| 185 | 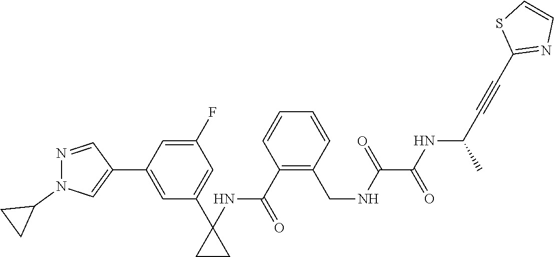 [SMILES: C[C@@H](C#Cc1nccs1)NC(=O)C(=O)NCc1ccccc1C(=O)NC1(c2cc(F)cc(-c3cnn(C4CC4)c3)c2)CC1] | 597.26 |
| 186 | 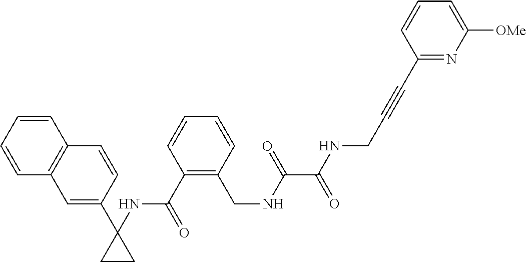 [SMILES: COc1cccc(C#CCNC(=O)C(=O)NCc2ccccc2C(=O)NC2(c3ccc4ccccc4c3)CC2)n1] | 533.32 |
| 187 | 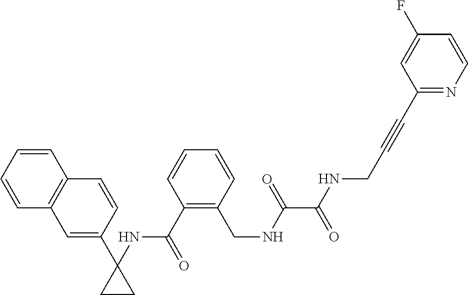 [SMILES: O=C(NCC#Cc1cc(F)ccn1)C(=O)NCc1ccccc1C(=O)NC1(c2ccc3ccccc3c2)CC1] | 521.32 |
| 188 | 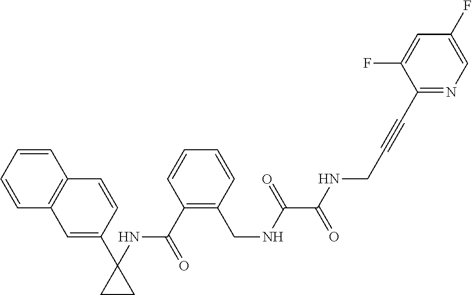 [SMILES: O=C(NCC#Cc1ncc(F)cc1F)C(=O)NCc1ccccc1C(=O)NC1(c2ccc3ccccc3c2)CC1] | 539.37 |
| 189 | 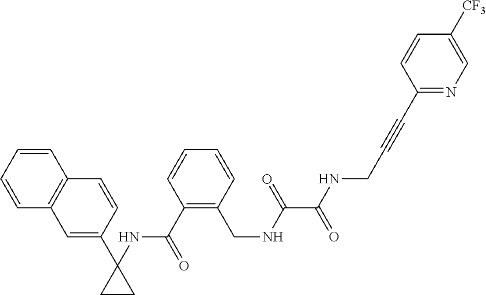 [SMILES: O=C(NCC#Cc1ccc(C(F)(F)F)cn1)C(=O)NCc1ccccc1C(=O)NC1(c2ccc3ccccc3c2)CC1] | 571.24 |
| 190 | 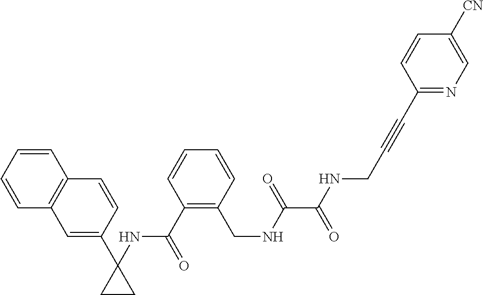 [SMILES: N#Cc1ccc(C#CCNC(=O)C(=O)NCc2ccccc2C(=O)NC2(c3ccc4ccccc4c3)CC2)nc1] | 528.28 |
| 191 | 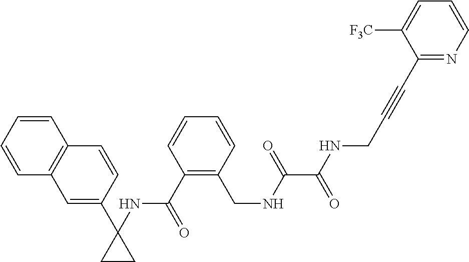 [SMILES: O=C(NCC#Cc1ncccc1C(F)(F)F)C(=O)NCc1ccccc1C(=O)NC1(c2ccc3ccccc3c2)CC1] | 571.24 |
| 192 | 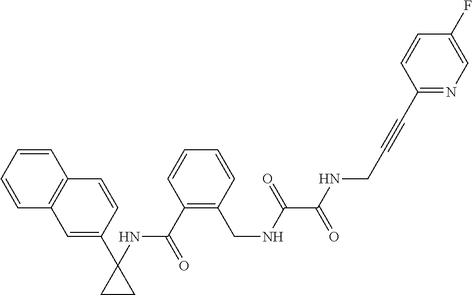 [SMILES: O=C(NCC#Cc1ccc(F)cn1)C(=O)NCc1ccccc1C(=O)NC1(c2ccc3ccccc3c2)CC1] | 521.23 |
| 193 | 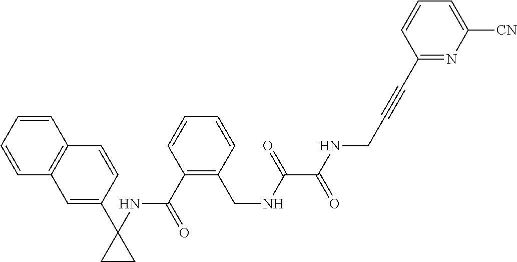 [SMILES: N#Cc1cccc(C#CCNC(=O)C(=O)NCc2ccccc2C(=O)NC2(c3ccc4ccccc4c3)CC2)n1] | 528.14 |
| 194 | 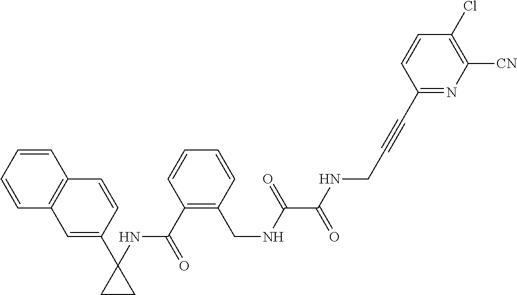 [SMILES: N#Cc1nc(C#CCNC(=O)C(=O)NCc2ccccc2C(=O)NC2(c3ccc4ccccc4c3)CC2)ccc1Cl] | 564.09 |
| 195 | 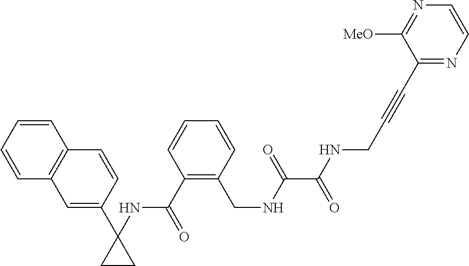 [SMILES: COc1nccnc1C#CCNC(=O)C(=O)NCc1ccccc1C(=O)NC1(c2ccc3ccccc3c2)CC1] | 534.28 |
| 196 | 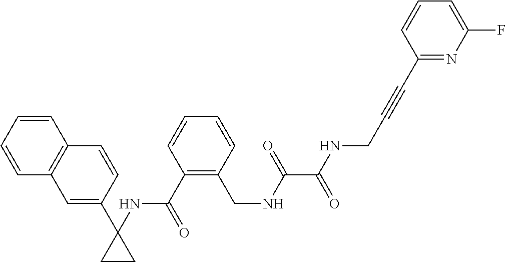 [SMILES: O=C(NCC#Cc1cccc(F)n1)C(=O)NCc1ccccc1C(=O)NC1(c2ccc3ccccc3c2)CC1] | 521.23 |
| 197 | 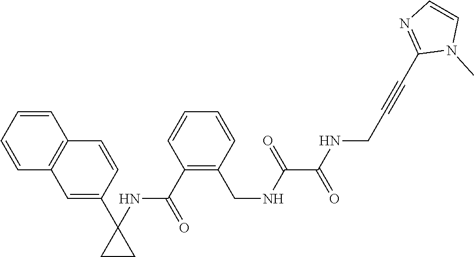 [SMILES: Cn1ccnc1C#CCNC(=O)C(=O)NCc1ccccc1C(=O)NC1(c2ccc3ccccc3c2)CC1] | 506.23 |
| 198 | 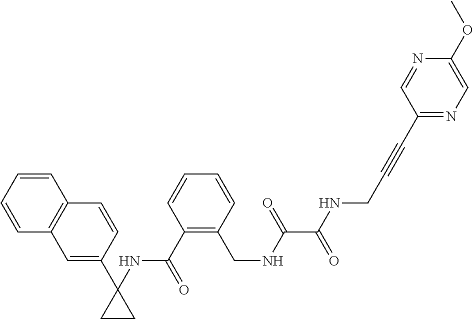 [SMILES: COc1cnc(C#CCNC(=O)C(=O)NCc2ccccc2C(=O)NC2(c3ccc4ccccc4c3)CC2)cn1] | 534.33 |
| 199 | 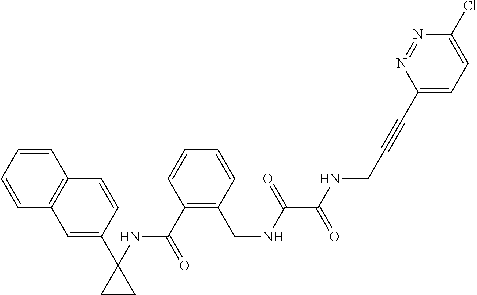 [SMILES: O=C(NCC#Cc1ccc(Cl)nn1)C(=O)NCc1ccccc1C(=O)NC1(c2ccc3ccccc3c2)CC1] | 540.14 |
| 200 | 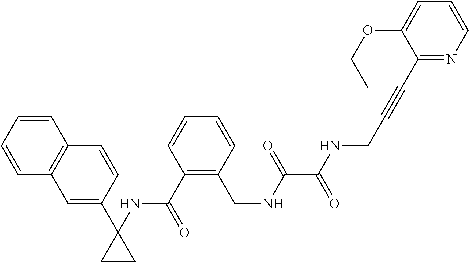 [SMILES: CCOc1cccnc1C#CCNC(=O)C(=O)NCc1ccccc1C(=O)NC1(c2ccc3ccccc3c2)CC1] | 547.34 |
| 201 | 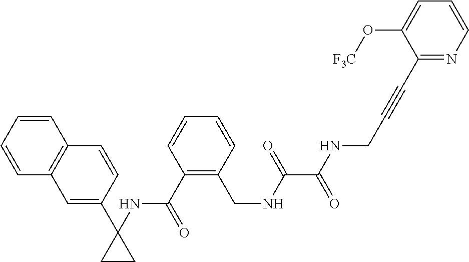 [SMILES: O=C(NCC#Cc1ncccc1OC(F)(F)F)C(=O)NCc1ccccc1C(=O)NC1(c2ccc3ccccc3c2)CC1] | 587.28 |
| 202 | 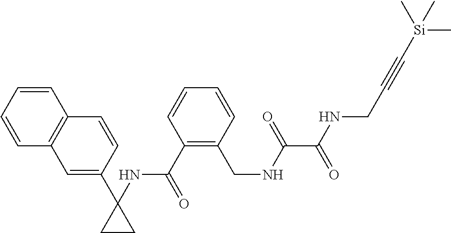 [SMILES: C[Si](C)(C)C#CCNC(=O)C(=O)NCc1ccccc1C(=O)NC1(c2ccc3ccccc3c2)CC1] | 498.14 |
| 203 | 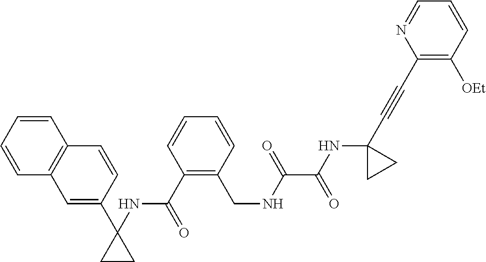 [SMILES: CCOc1cccnc1C#CC1(NC(=O)C(=O)NCc2ccccc2C(=O)NC2(c3ccc4ccccc4c3)CC2)CC1] | 573.21 |
| 204 | 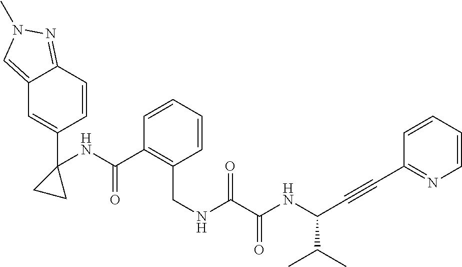 [SMILES: CC(C)[C@@H](C#Cc1ccccn1)NC(=O)C(=O)NCc1ccccc1C(=O)NC1(c2ccc3nn(C)cc3c2)CC1] | 549.21 |
| 205 | 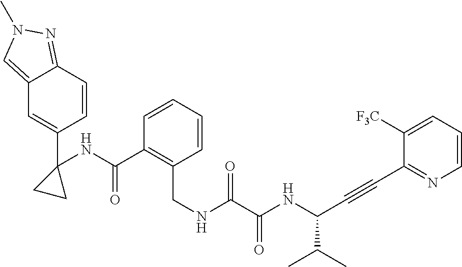 [SMILES: CC(C)[C@@H](C#Cc1ncccc1C(F)(F)F)NC(=O)C(=O)NCc1ccccc1C(=O)NC1(c2ccc3nn(C)cc3c2)CC1] | 617.33 |
| 206 | 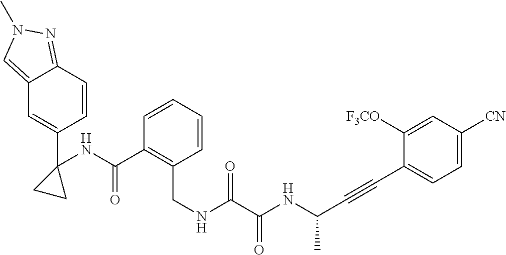 [SMILES: C[C@@H](C#Cc1ccc(C#N)cc1OC(F)(F)F)NC(=O)C(=O)NCc1ccccc1C(=O)NC1(c2ccc3nn(C)cc3c2)CC1] | 629.28 |
| 207 | 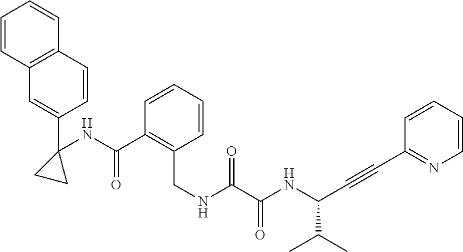 [SMILES: CC(C)[C@@H](C#Cc1ccccn1)NC(=O)C(=O)NCc1ccccc1C(=O)NC1(c2ccc3ccccc3c2)CC1] | 545.32 |
| 208 | 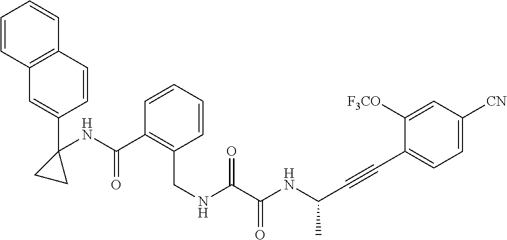 [SMILES: C[C@@H](C#Cc1ccc(C#N)cc1OC(F)(F)F)NC(=O)C(=O)NCc1ccccc1C(=O)NC1(c2ccc3ccccc3c2)CC1] | 625.29 |
| 209 | 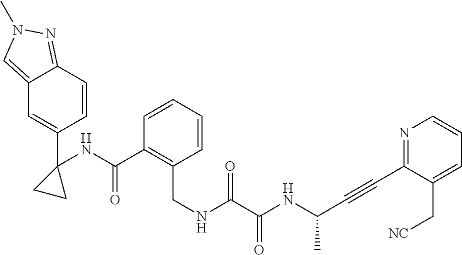 [SMILES: C[C@@H](C#Cc1ncccc1CC#N)NC(=O)C(=O)NCc1ccccc1C(=O)NC1(c2ccc3nn(C)cc3c2)CC1] | 560.30 |
| 210 | 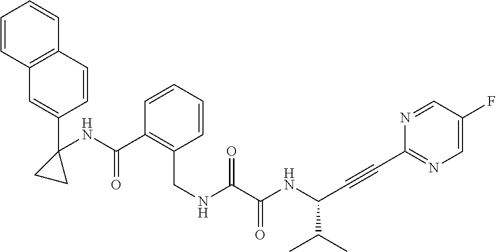 [SMILES: CC(C)[C@@H](C#Cc1ncc(F)cn1)NC(=O)C(=O)NCc1ccccc1C(=O)NC1(c2ccc3ccccc3c2)CC1] | 564.33 |
| 211 | 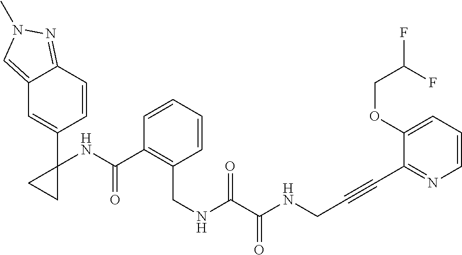 [SMILES: Cn1cc2cc(C3(NC(=O)c4ccccc4CNC(=O)C(=O)NCC#Cc4ncccc4OCC(F)F)CC3)ccc2n1] | 587.28 |
| 212 | 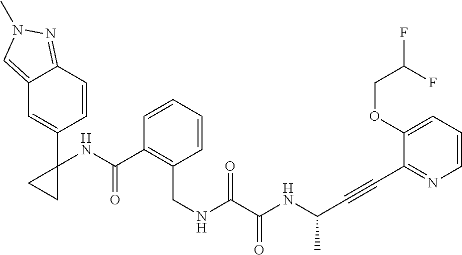 [SMILES: C[C@@H](C#Cc1ncccc1OCC(F)F)NC(=O)C(=O)NCc1ccccc1C(=O)NC1(c2ccc3nn(C)cc3c2)CC1] | 601.39 |
| 213 | 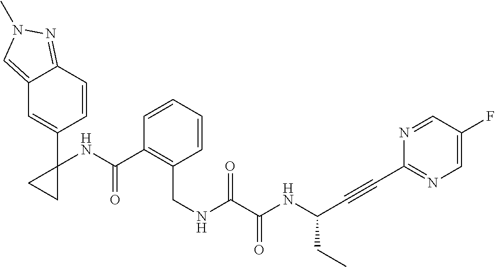 [SMILES: CC[C@@H](C#Cc1ncc(F)cn1)NC(=O)C(=O)NCc1ccccc1C(=O)NC1(c2ccc3nn(C)cc3c2)CC1] | 554.35 |
| 214 | 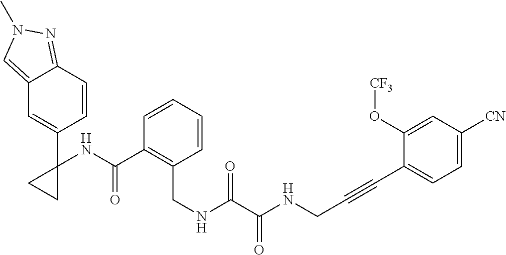 [SMILES: Cn1cc2cc(C3(NC(=O)c4ccccc4CNC(=O)C(=O)NCC#Cc4ccc(C#N)cc4OC(F)(F)F)CC3)ccc2n1] | 615.26 |
| 215 | 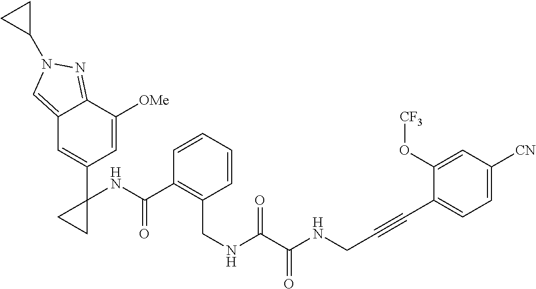 [SMILES: COc1cc(C2(NC(=O)c3ccccc3CNC(=O)C(=O)NCC#Cc3ccc(C#N)cc3OC(F)(F)F)CC2)cc2cn(C3CC3)nc12] | 671.33 |
| 216 | 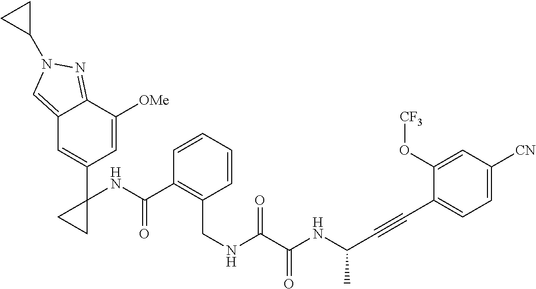 [SMILES: COc1cc(C2(NC(=O)c3ccccc3CNC(=O)C(=O)N[C@@H](C)C#Cc3ccc(C#N)cc3OC(F)(F)F)CC2)cc2cn(C3CC3)nc12] | 685.25 |
| 217 | 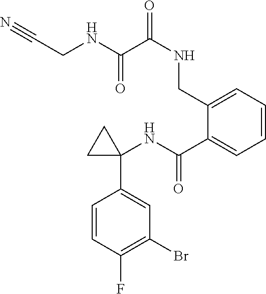 [SMILES: N#CCNC(=O)C(=O)NCc1ccccc1C(=O)NC1(c2ccc(F)c(Br)c2)CC1] | 473.05 |
| 218 | 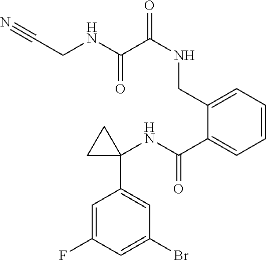 [SMILES: N#CCNC(=O)C(=O)NCc1ccccc1C(=O)NC1(c2cc(F)cc(Br)c2)CC1] | 473.05 |
| 219 | 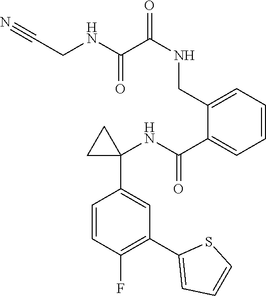 [SMILES: N#CCNC(=O)C(=O)NCc1ccccc1C(=O)NC1(c2ccc(F)c(-c3cccs3)c2)CC1] | 477.18 |
| 220 | 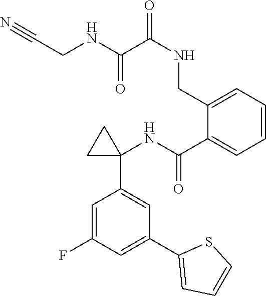 [SMILES: N#CCNC(=O)C(=O)NCc1ccccc1C(=O)NC1(c2cc(F)cc(-c3cccs3)c2)CC1] | 477.15 |
| 221 | 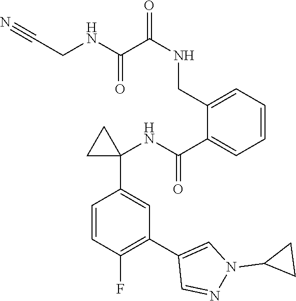 [SMILES: N#CCNC(=O)C(=O)NCc1ccccc1C(=O)NC1(c2ccc(F)c(-c3cnn(C4CC4)c3)c2)CC1] | 501.20 |
| 222 | 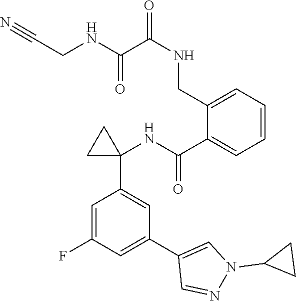 [SMILES: N#CCNC(=O)C(=O)NCc1ccccc1C(=O)NC1(c2cc(F)cc(-c3cnn(C4CC4)c3)c2)CC1] | 501.19 |
| 223 | 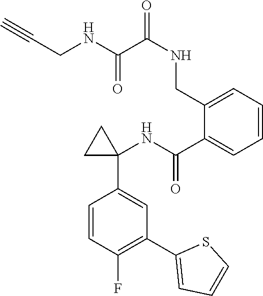 [SMILES: C#CCNC(=O)C(=O)NCc1ccccc1C(=O)NC1(c2ccc(F)c(-c3cccs3)c2)CC1] | 476.17 |
| 224 | 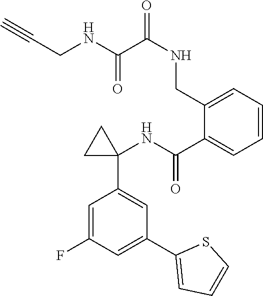 [SMILES: C#CCNC(=O)C(=O)NCc1ccccc1C(=O)NC1(c2cc(F)cc(-c3cccs3)c2)CC1] | 476.16 |
| 225 | 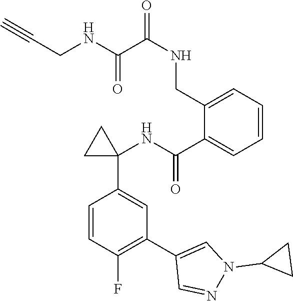 [SMILES: C#CCNC(=O)C(=O)NCc1ccccc1C(=O)NC1(c2ccc(F)c(-c3cnn(C4CC4)c3)c2)CC1] | 500.19 |
| 226 | 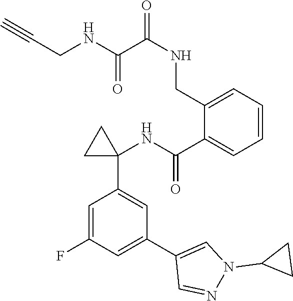 [SMILES: C#CCNC(=O)C(=O)NCc1ccccc1C(=O)NC1(c2cc(F)cc(-c3cnn(C4CC4)c3)c2)CC1] | 500.20 |
| 227 | 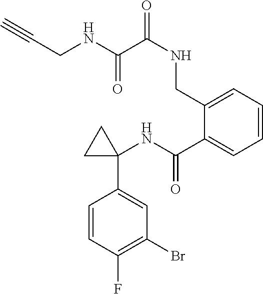 [SMILES: C#CCNC(=O)C(=O)NCc1ccccc1C(=O)NC1(c2ccc(F)c(Br)c2)CC1] | 472.05 |
| 228 | 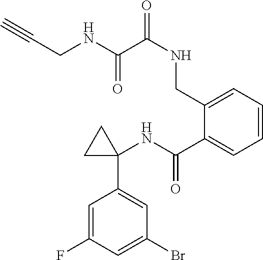 [SMILES: C#CCNC(=O)C(=O)NCc1ccccc1C(=O)NC1(c2cc(F)cc(Br)c2)CC1] | 472.05 |
| 229 | 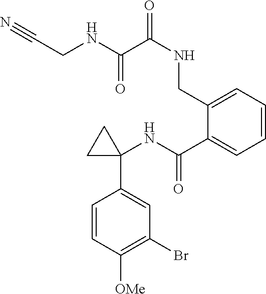 [SMILES: COc1ccc(C2(NC(=O)c3ccccc3CNC(=O)C(=O)NCC#N)CC2)cc1Br] | 485.08 |
| 230 | 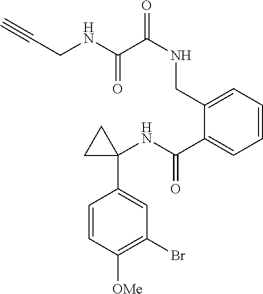 [SMILES: C#CCNC(=O)C(=O)NCc1ccccc1C(=O)NC1(c2ccc(OC)c(Br)c2)CC1] | 484.09 |
| 231 | 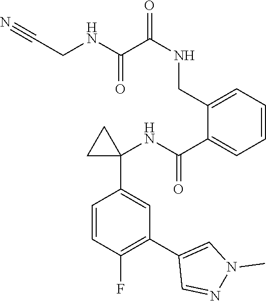 [SMILES: Cn1cc(-c2cc(C3(NC(=O)c4ccccc4CNC(=O)C(=O)NCC#N)CC3)ccc2F)cn1] | 475.18 |
| 232 | 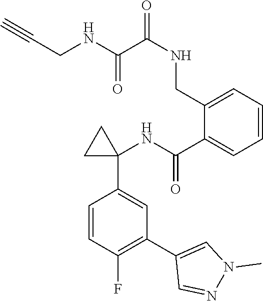 [SMILES: C#CCNC(=O)C(=O)NCc1ccccc1C(=O)NC1(c2ccc(F)c(-c3cnn(C)c3)c2)CC1] | 474.18 |
| 233 | 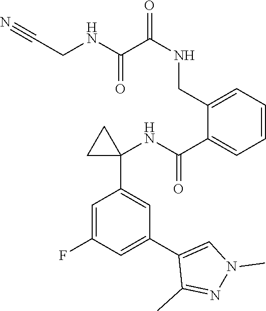 [SMILES: Cc1nn(C)cc1-c1cc(F)cc(C2(NC(=O)c3ccccc3CNC(=O)C(=O)NCC#N)CC2)c1] | 489.19 |
| 234 | 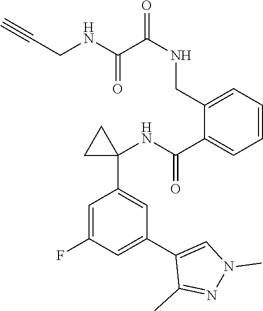 [SMILES: C#CCNC(=O)C(=O)NCc1ccccc1C(=O)NC1(c2cc(F)cc(-c3cn(C)nc3C)c2)CC1] | 488.20 |
| 235 | 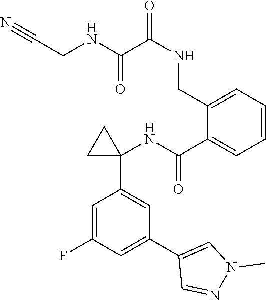 [SMILES: Cn1cc(-c2cc(F)cc(C3(NC(=O)c4ccccc4CNC(=O)C(=O)NCC#N)CC3)c2)cn1] | 475.18 |
| 236 | 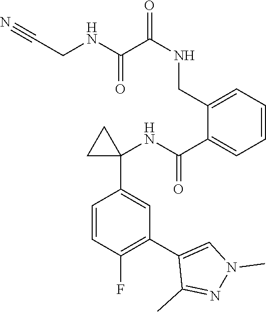 [SMILES: Cc1nn(C)cc1-c1cc(C2(NC(=O)c3ccccc3CNC(=O)C(=O)NCC#N)CC2)ccc1F] | 489.17 |
| 237 | 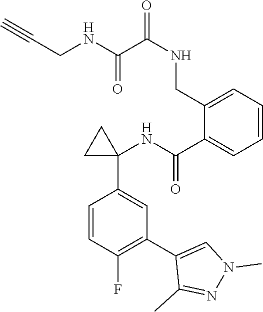 [SMILES: C#CCNC(=O)C(=O)NCc1ccccc1C(=O)NC1(c2ccc(F)c(-c3cn(C)nc3C)c2)CC1] | 488.20 |
| 238 | 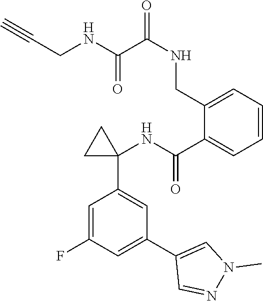 [SMILES: C#CCNC(=O)C(=O)NCc1ccccc1C(=O)NC1(c2cc(F)cc(-c3cnn(C)c3)c2)CC1] | 474.19 |
| 239 | 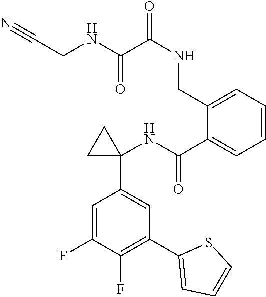 [SMILES: N#CCNC(=O)C(=O)NCc1ccccc1C(=O)NC1(c2cc(F)c(F)c(-c3cccs3)c2)CC1] | 495.13 |
| 240 | 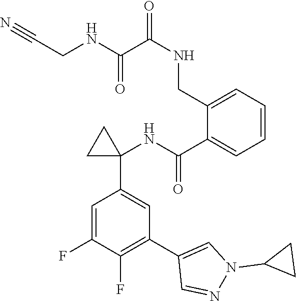 [SMILES: N#CCNC(=O)C(=O)NCc1ccccc1C(=O)NC1(c2cc(F)c(F)c(-c3cnn(C4CC4)c3)c2)CC1] | 519.18 |
| 241 | 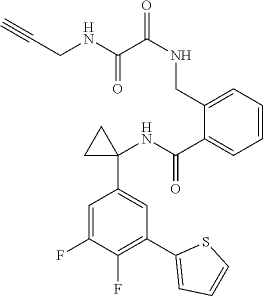 [SMILES: C#CCNC(=O)C(=O)NCc1ccccc1C(=O)NC1(c2cc(F)c(F)c(-c3cccs3)c2)CC1] | 494.13 |
| 242 | 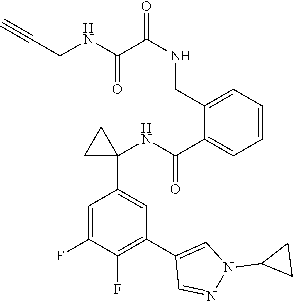 [SMILES: C#CCNC(=O)C(=O)NCc1ccccc1C(=O)NC1(c2cc(F)c(F)c(-c3cnn(C4CC4)c3)c2)CC1] | 518.18 |
| 243 | 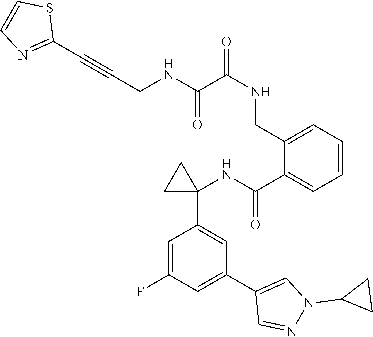 [SMILES: O=C(NCC#Cc1nccs1)C(=O)NCc1ccccc1C(=O)NC1(c2cc(F)cc(-c3cnn(C4CC4)c3)c2)CC1] | 583.17 |
| 244 | 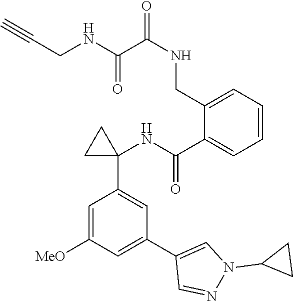 [SMILES: C#CCNC(=O)C(=O)NCc1ccccc1C(=O)NC1(c2cc(OC)cc(-c3cnn(C4CC4)c3)c2)CC1] | 512.22 |
| 245 | 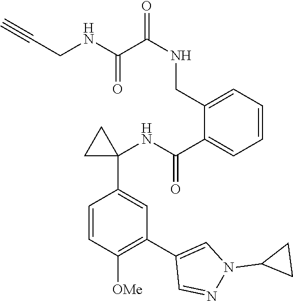 [SMILES: C#CCNC(=O)C(=O)NCc1ccccc1C(=O)NC1(c2ccc(OC)c(-c3cnn(C4CC4)c3)c2)CC1] | 512.22 |
| 246 | 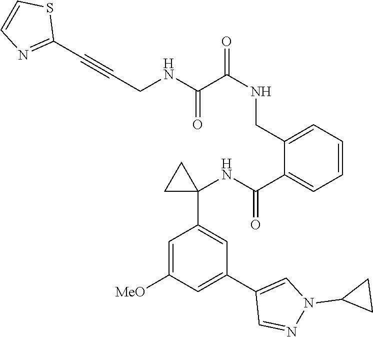 [SMILES: COc1cc(-c2cnn(C3CC3)c2)cc(C2(NC(=O)c3ccccc3CNC(=O)C(=O)NCC#Cc3nccs3)CC2)c1] | 595.19 |
| 247 | 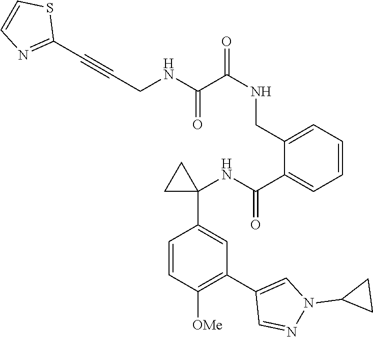 [SMILES: COc1ccc(C2(NC(=O)c3ccccc3CNC(=O)C(=O)NCC#Cc3nccs3)CC2)cc1-c1cnn(C2CC2)c1] | 595.19 |
| 248 | 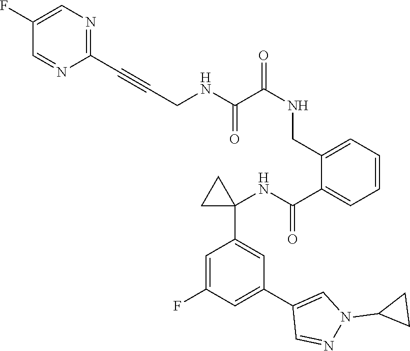 [SMILES: O=C(NCC#Cc1ncc(F)cn1)C(=O)NCc1ccccc1C(=O)NC1(c2cc(F)cc(-c3cnn(C4CC4)c3)c2)CC1] | 596.20 |
| 249 | 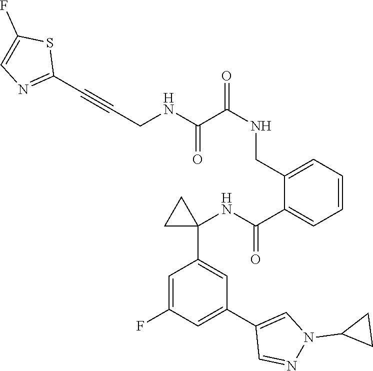 [SMILES: O=C(NCC#Cc1ncc(F)s1)C(=O)NCc1ccccc1C(=O)NC1(c2cc(F)cc(-c3cnn(C4CC4)c3)c2)CC1] | 601.17 |
| 250 | 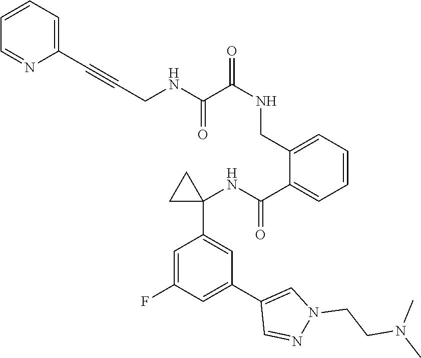 [SMILES: CN(C)CCn1cc(-c2cc(F)cc(C3(NC(=O)c4ccccc4CNC(=O)C(=O)NCC#Cc4ccccn4)CC3)c2)cn1] | 608.32 |
| 251 | 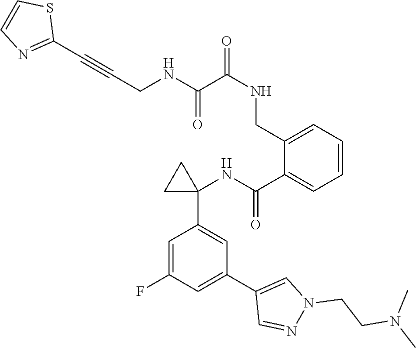 [SMILES: CN(C)CCn1cc(-c2cc(F)cc(C3(NC(=O)c4ccccc4CNC(=O)C(=O)NCC#Cc4nccs4)CC3)c2)cn1] | 614.21 |
| 252 | 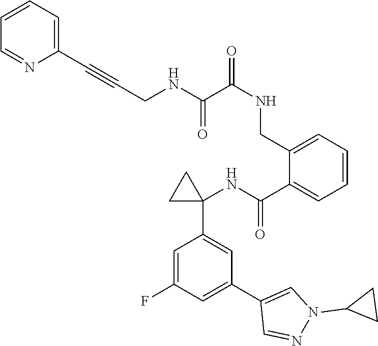 [SMILES: O=C(NCC#Cc1ccccn1)C(=O)NCc1ccccc1C(=O)NC1(c2cc(F)cc(-c3cnn(C4CC4)c3)c2)CC1] | 577.21 |
| 253 | 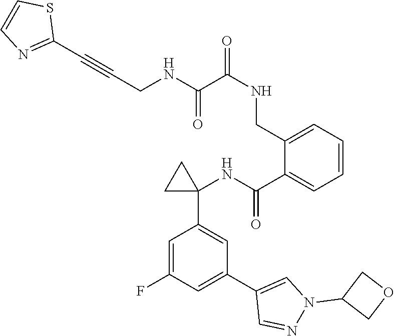 [SMILES: O=C(NCC#Cc1nccs1)C(=O)NCc1ccccc1C(=O)NC1(c2cc(F)cc(-c3cnn(C4COC4)c3)c2)CC1] | 599.18 |
| 254 | 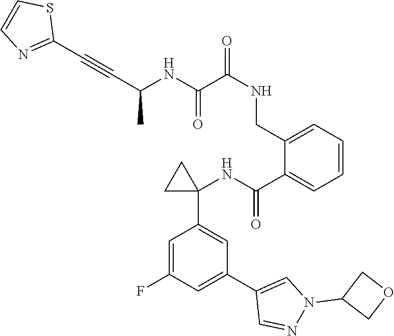 [SMILES: C[C@@H](C#Cc1nccs1)NC(=O)C(=O)NCc1ccccc1C(=O)NC1(c2cc(F)cc(-c3cnn(C4COC4)c3)c2)CC1] | 613.19 |
| 255 | 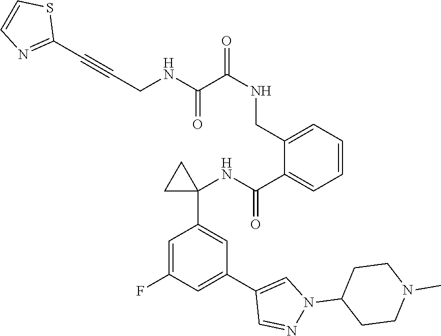 [SMILES: CN1CCC(n2cc(-c3cc(F)cc(C4(NC(=O)c5ccccc5CNC(=O)C(=O)NCC#Cc5nccs5)CC4)c3)cn2)CC1] | 640.22 |
| 256 | 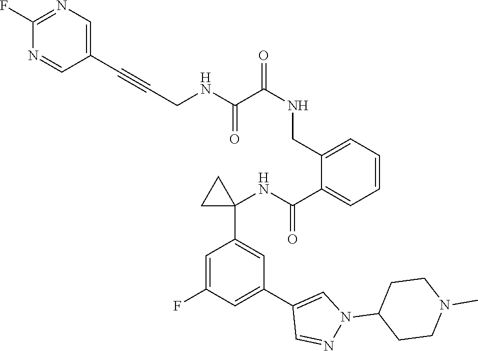 [SMILES: CN1CCC(n2cc(-c3cc(F)cc(C4(NC(=O)c5ccccc5CNC(=O)C(=O)NCC#Cc5cnc(F)nc5)CC4)c3)cn2)CC1] | 651.28 (M − H)⁻ |
| 257 | 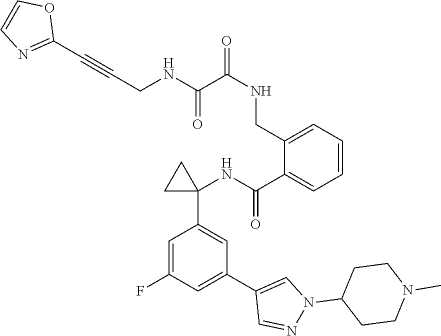 [SMILES: CN1CCC(n2cc(-c3cc(F)cc(C4(NC(=O)c5ccccc5CNC(=O)C(=O)NCC#Cc5ncco5)CC4)c3)cn2)CC1] | 624.24 |
| 258 | 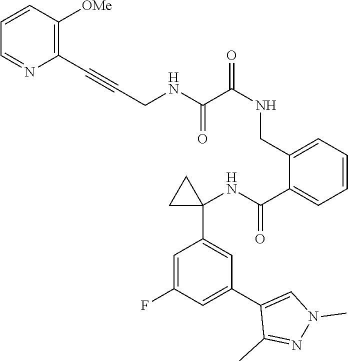 [SMILES: COc1cccnc1C#CCNC(=O)C(=O)NCc1ccccc1C(=O)NC1(c2cc(F)cc(-c3cn(C)nc3C)c2)CC1] | 595.20 |
| 259 | 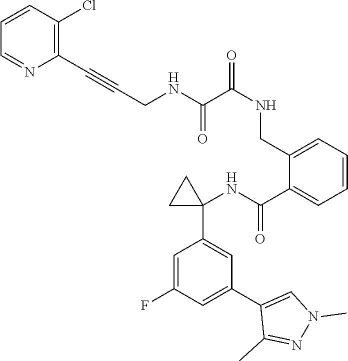 [SMILES: Cc1nn(C)cc1-c1cc(F)cc(C2(NC(=O)c3ccccc3CNC(=O)C(=O)NCC#Cc3ncccc3Cl)CC2)c1] | 599.19 |
| 260 | 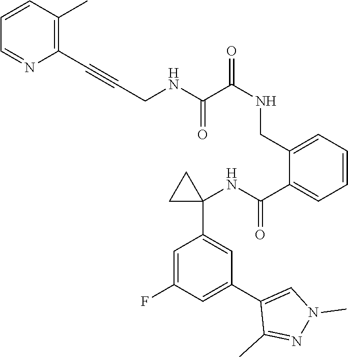 [SMILES: Cc1cccnc1C#CCNC(=O)C(=O)NCc1ccccc1C(=O)NC1(c2cc(F)cc(-c3cn(C)nc3C)c2)CC1] | 579.22 |
| 261 | 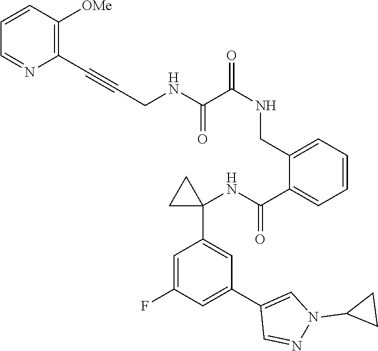 [SMILES: COc1cccnc1C#CCNC(=O)C(=O)NCc1ccccc1C(=O)NC1(c2cc(F)cc(-c3cnn(C4CC4)c3)c2)CC1] | 607.23 |
| 262 | 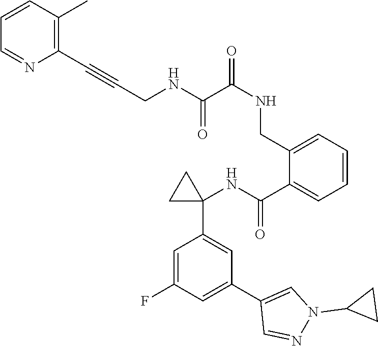 [SMILES: Cc1cccnc1C#CCNC(=O)C(=O)NCc1ccccc1C(=O)NC1(c2cc(F)cc(-c3cnn(C4CC4)c3)c2)CC1] | 591.22 |
| 263 | 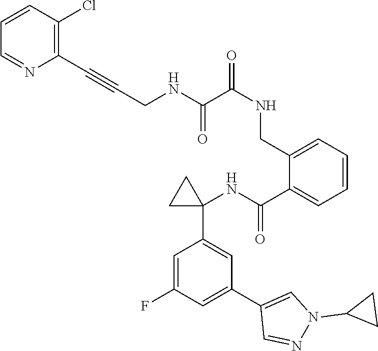 [SMILES: O=C(NCC#Cc1ncccc1Cl)C(=O)NCc1ccccc1C(=O)NC1(c2cc(F)cc(-c3cnn(C4CC4)c3)c2)CC1] | 612.20 |
| 264 | 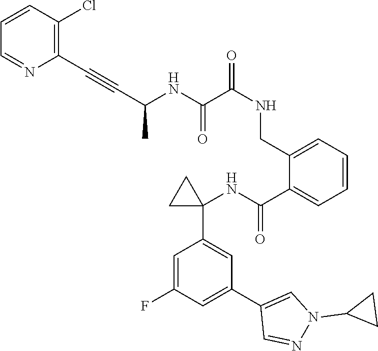 [SMILES: C[C@@H](C#Cc1ncccc1Cl)NC(=O)C(=O)NCc1ccccc1C(=O)NC1(c2cc(F)cc(-c3cnn(C4CC4)c3)c2)CC1] | 625.19 |
| 265 | 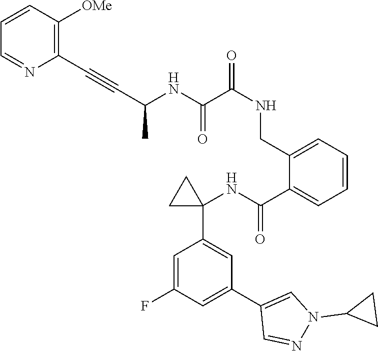 [SMILES: COc1cccnc1C#C[C@H](C)NC(=O)C(=O)NCc1ccccc1C(=O)NC1(c2cc(F)cc(-c3cnn(C4CC4)c3)c2)CC1] | 621.23 |
| 266 | 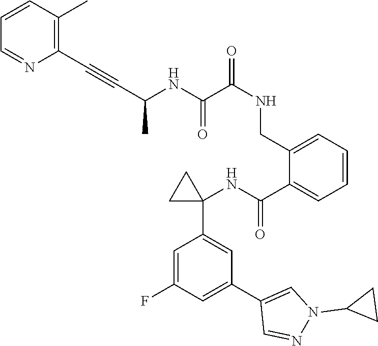 [SMILES: Cc1cccnc1C#C[C@H](C)NC(=O)C(=O)NCc1ccccc1C(=O)NC1(c2cc(F)cc(-c3cnn(C4CC4)c3)c2)CC1] | 605.24 |
| 267 | 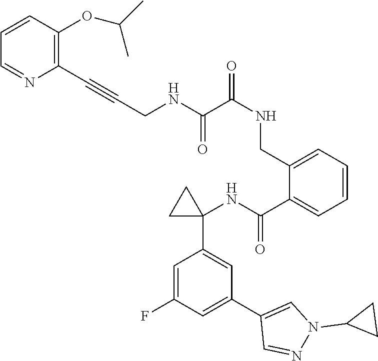 [SMILES: CC(C)Oc1cccnc1C#CCNC(=O)C(=O)NCc1ccccc1C(=O)NC1(c2cc(F)cc(-c3cnn(C4CC4)c3)c2)CC1] | 635.53 |
| 268 | 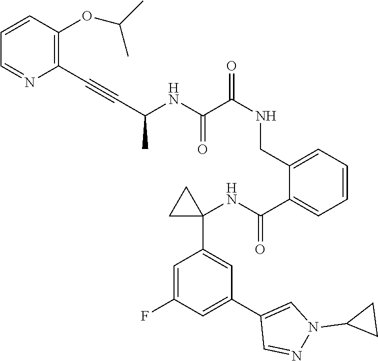 [SMILES: CC(C)Oc1cccnc1C#C[C@H](C)NC(=O)C(=O)NCc1ccccc1C(=O)NC1(c2cc(F)cc(-c3cnn(C4CC4)c3)c2)CC1] | 649.53 |
| 269 | 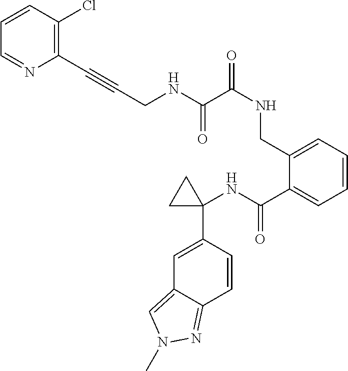 [SMILES: Cn1cc2cc(C3(NC(=O)c4ccccc4CNC(=O)C(=O)NCC#Cc4ncccc4Cl)CC3)ccc2n1] | 541.16 |
| 270 | 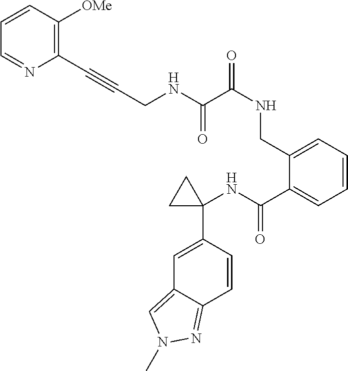 [SMILES: COc1cccnc1C#CCNC(=O)C(=O)NCc1ccccc1C(=O)NC1(c2ccc3nn(C)cc3c2)CC1] | 537.26 |
| 271 | 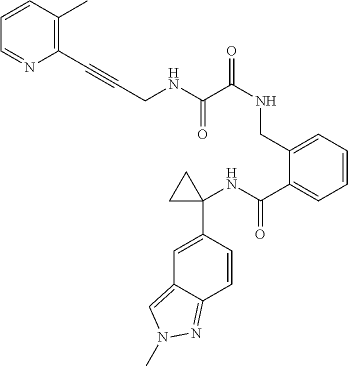 [SMILES: Cc1cccnc1C#CCNC(=O)C(=O)NCc1ccccc1C(=O)NC1(c2ccc3nn(C)cc3c2)CC1] | 521.26 |
| 272 | 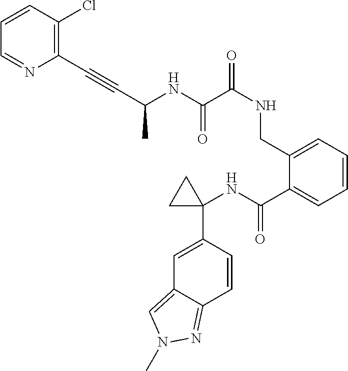 [SMILES: C[C@@H](C#Cc1ncccc1Cl)NC(=O)C(=O)NCc1ccccc1C(=O)NC1(c2ccc3nn(C)cc3c2)CC1] | 555.17 |
| 273 | 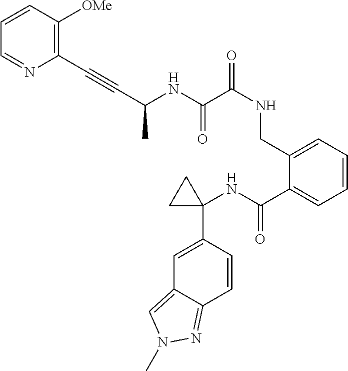 [SMILES: COc1cccnc1C#C[C@H](C)NC(=O)C(=O)NCc1ccccc1C(=O)NC1(c2ccc3nn(C)cc3c2)CC1] | 551.23 |
| 274 | 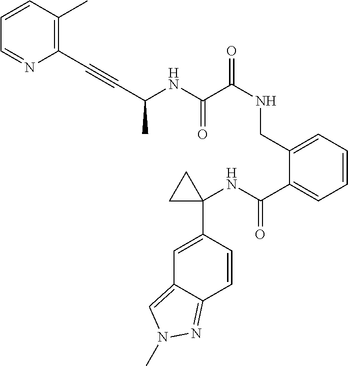 [SMILES: Cc1cccnc1C#C[C@H](C)NC(=O)C(=O)NCc1ccccc1C(=O)NC1(c2ccc3nn(C)cc3c2)CC1] | 535.28 |
| 275 | 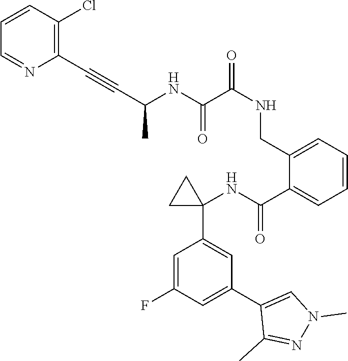 [SMILES: Cc1nn(C)cc1-c1cc(F)cc(C2(NC(=O)c3ccccc3CNC(=O)C(=O)N[C@@H](C)C#Cc3ncccc3Cl)CC2)c1] | 613.21 |
| 276 | 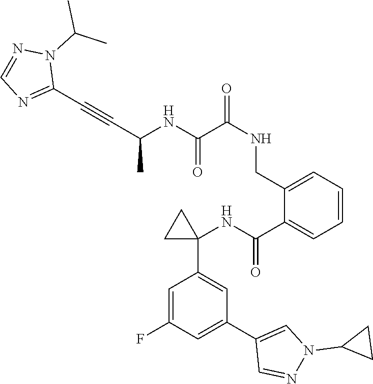 [SMILES: CC(C)n1ncnc1C#C[C@H](C)NC(=O)C(=O)NCc1ccccc1C(=O)NC1(c2cc(F)cc(-c3cnn(C4CC4)c3)c2)CC1] | 623.26 |
| 277 | 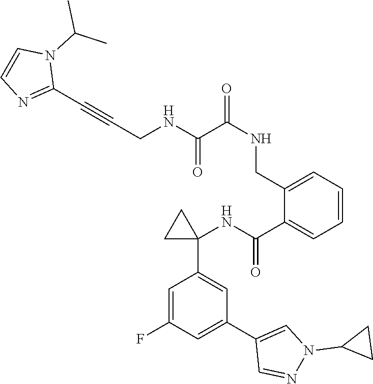 [SMILES: CC(C)n1ccnc1C#CCNC(=O)C(=O)NCc1ccccc1C(=O)NC1(c2cc(F)cc(-c3cnn(C4CC4)c3)c2)CC1] | 608.24 |
| 278 | 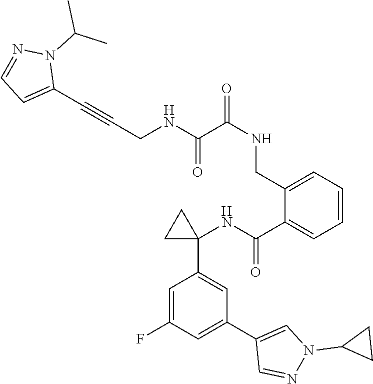 | 608.28 |
| 279 | 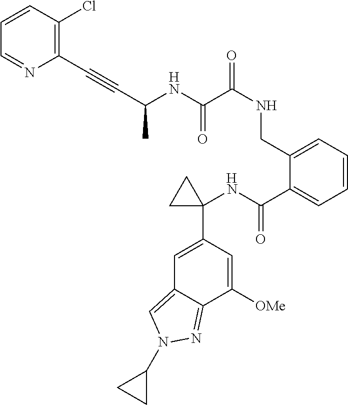 | 611.18 |
| 280 | 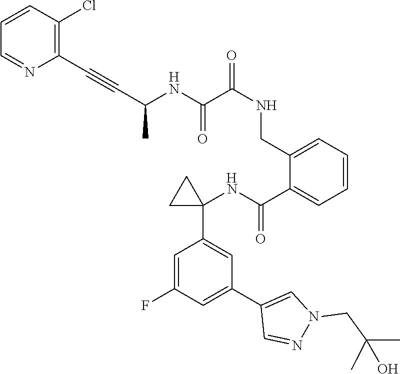 | 657.23 |
| 281 | 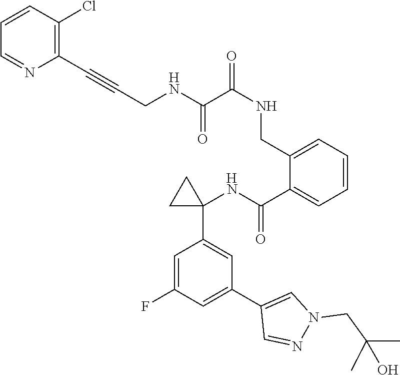 | 643.20 |
| 282 | 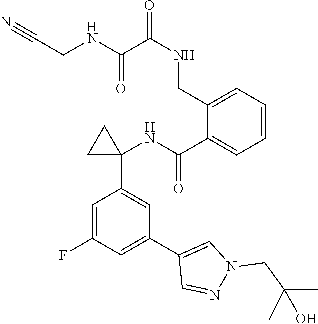 | 533.49 |
| 283 | 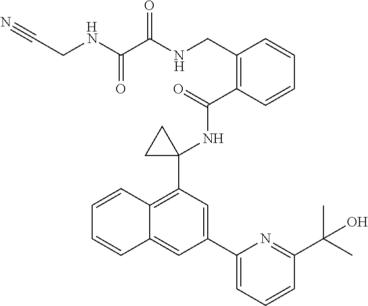 | 562.24 |
| 284 | 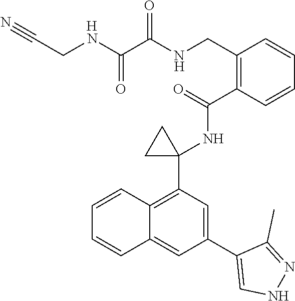 | 507.19 |
| 285 | 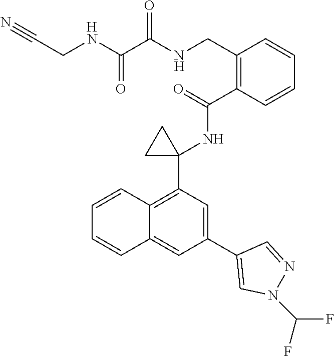 | 543.19 |
| 286 | 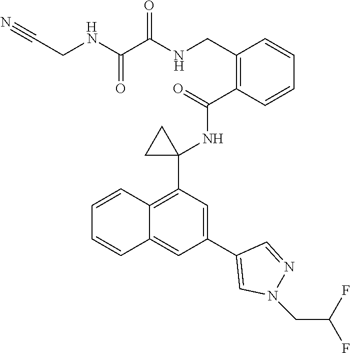 | 557.20 |
| 287 | 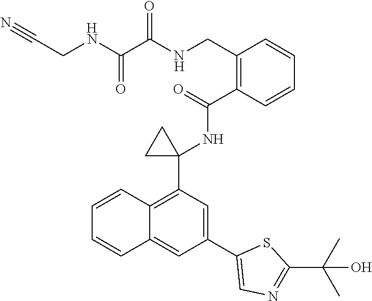 | 568.19 |
| 288 | 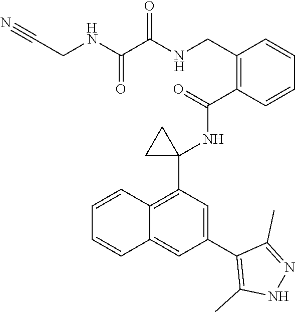 | 521.20 |
| 289 | 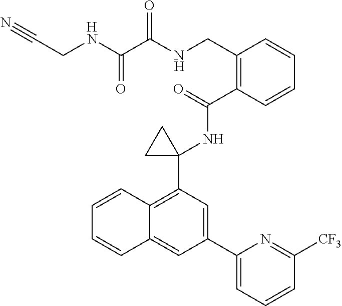 | 533.18 |
| 290 | 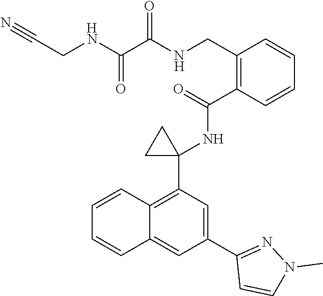 | 507.19 |
| 291 | 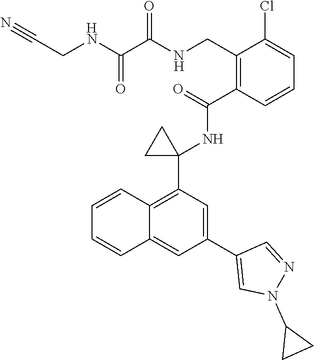 | 567.17 |
| 292 | 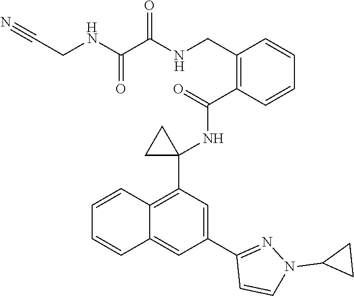 | 572.18 |
| 293 | 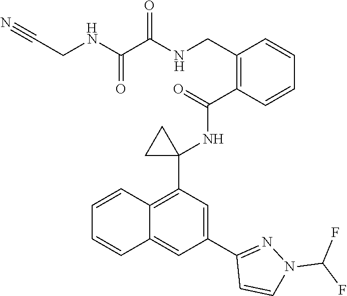 | 543.20 |
| 294 | 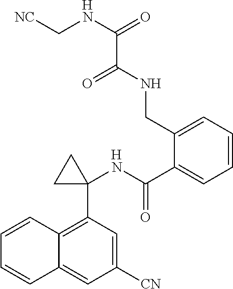 | 452.17 |
| 295 | 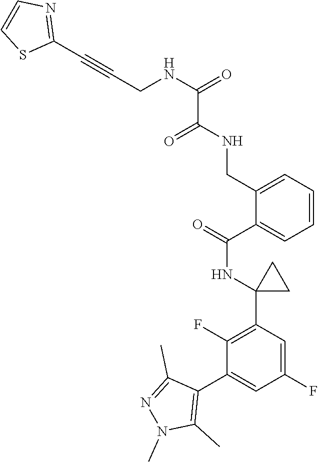 | 603.18 |
| 296 | 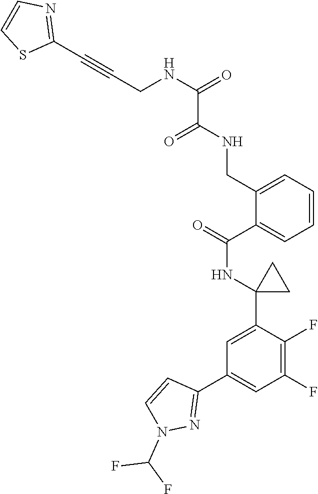 | 611.13 |
| 297 | 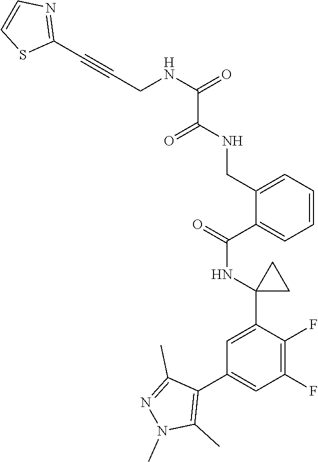 | 603.19 |
| 298 | 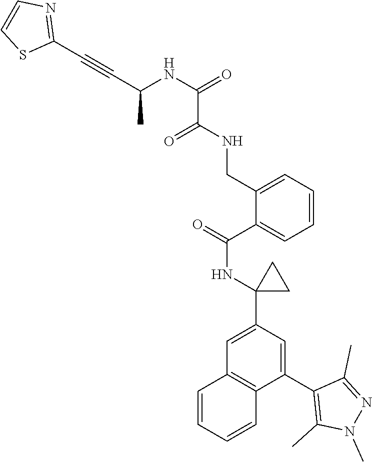 | 631.25 |
| 299 | 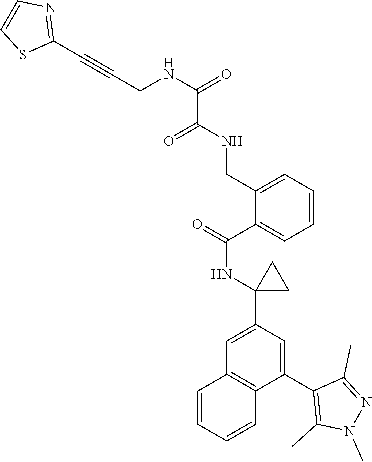 | 617.24 |
| 300 | 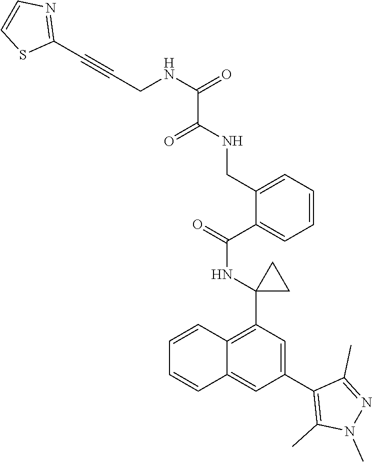 | 617.23 |
| 301 | 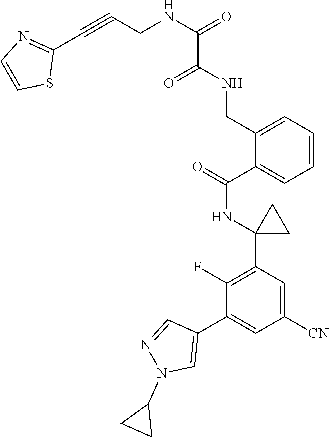 | 608.25 |
| 302 | 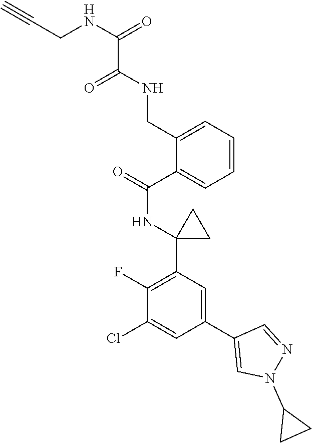 | 534.16 |
| 303 | 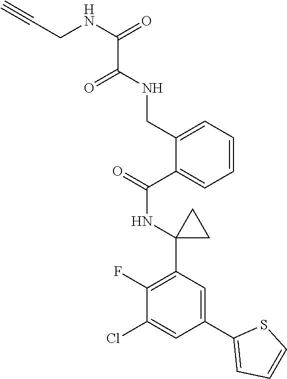 | 510.13 |
| 304 | 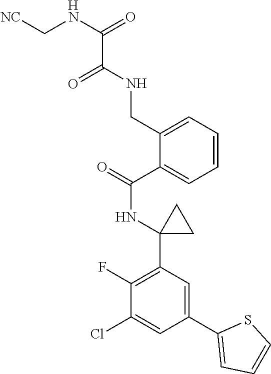 | 511.10 |
| 305 | 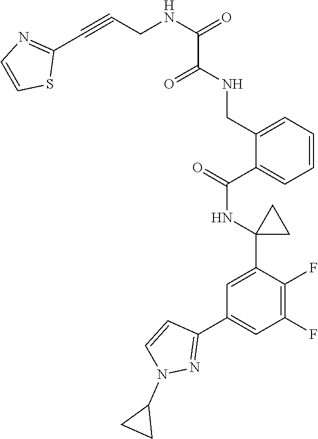 | 601.24 |
| 306 | 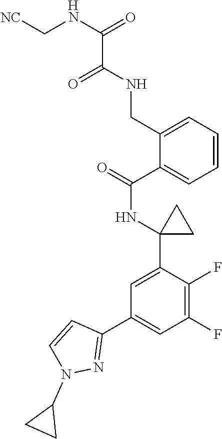 | 519.17 |
| 307 | 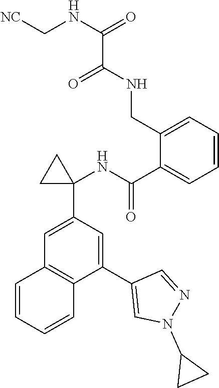 | 533.22 |
| 308 | 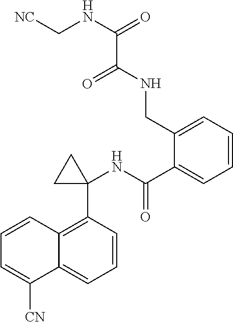 | 452.17 |
| 309 | 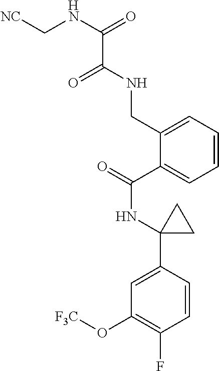 | 479.12 |
| 310 | 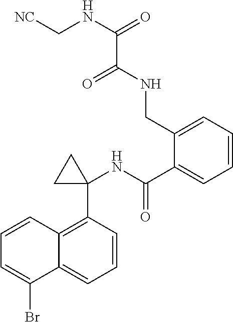 | 505.09 |
| 311 | 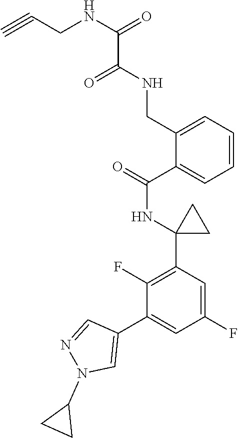 | 518.19 |
| 312 | 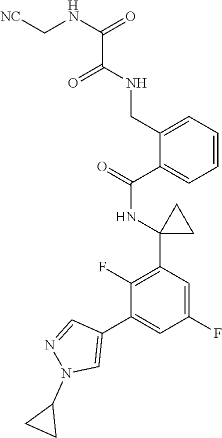 | 519.09 |
| 313 | 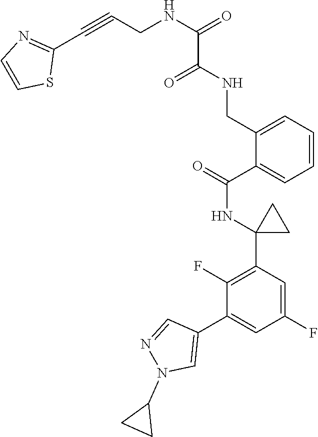 | 601.18 |
| 314 | 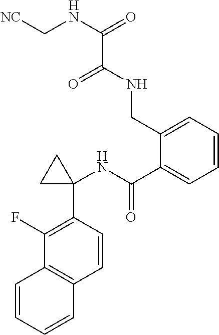 | 455.16 |
| 315 | 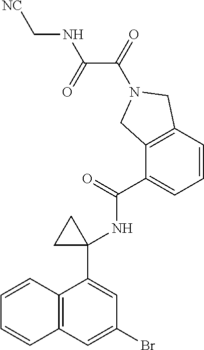 [SMILES: N#CCNC(=O)C(=O)N1Cc2cccc(C(=O)NC3(c4cc(Br)cc5ccccc45)CC3)c2C1] | 517.09 |
| 316 | 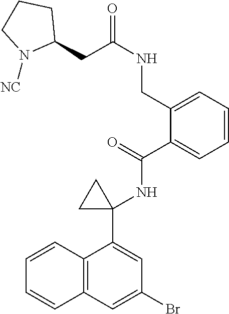 [SMILES: N#CN1CCC[C@H]1CC(=O)NCc1ccccc1C(=O)NC1(c2cc(Br)cc3ccccc23)CC1] | 531.19 |
| 317 | 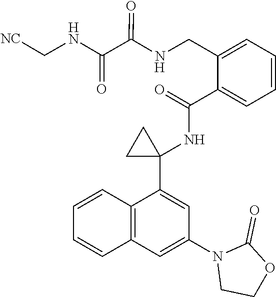 | 512.19 |
| 318 | 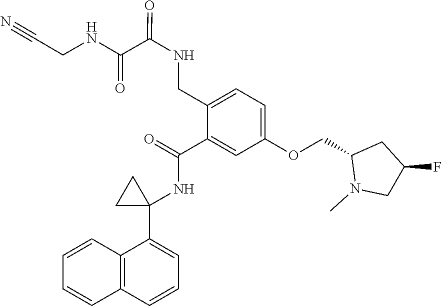 | 558.35 [M − H]⁻ |
| 319 | 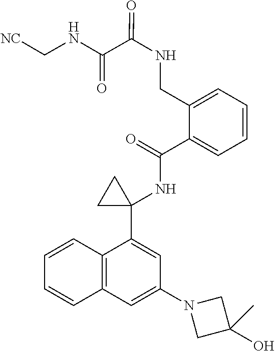 | 512.45 |
| 320 | 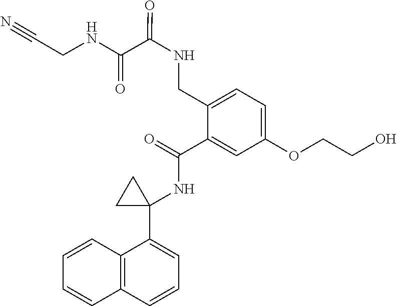 | 487.22 |
| 321 | 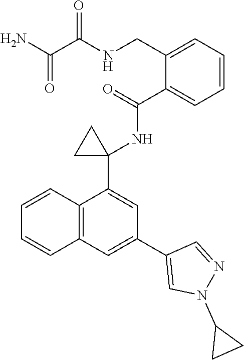 | 494.35 |
| 322 | 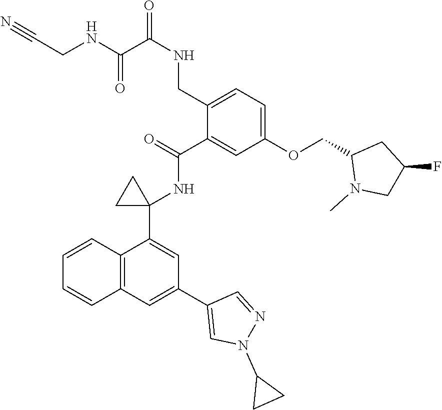 | 664.38 |
| 323 | 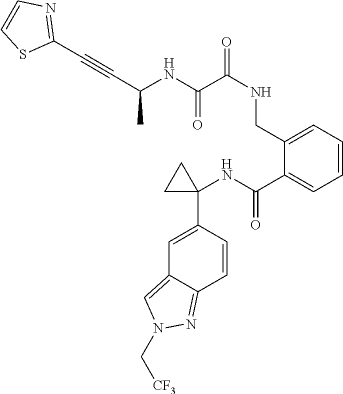 | 595.2 |
| 324 | 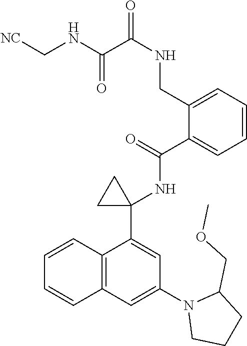 | 540.22 |
| 325 | 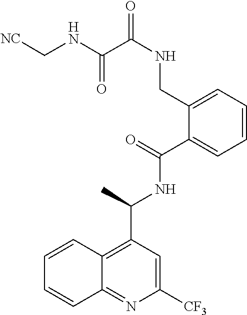 | 484.16 |
| 326 | 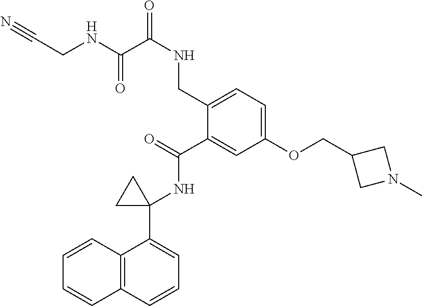 | 526.48 [M − H]⁻ |
| 327 | 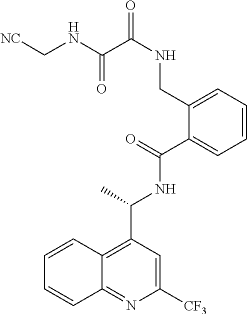 | 484.16 [M − H]⁻ |
| 328 | 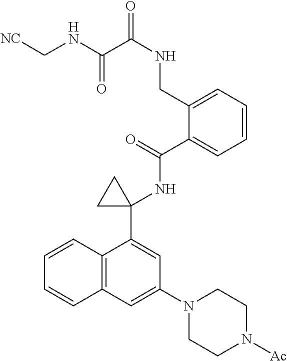 | 553.24 |
| 329 | 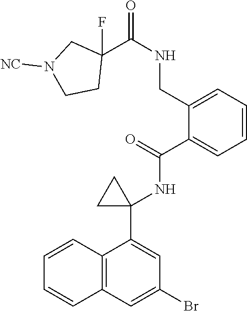 [SMILES: N#CN1CCC(F)(C(=O)NCc2ccccc2C(=O)NC2(c3cc(Br)cc4ccccc34)CC2)C1] | 535.15 |
| 330 | 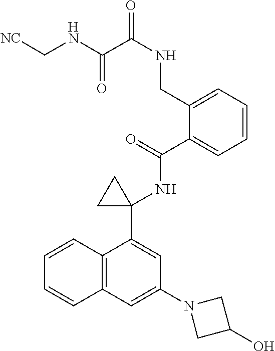 | 498.32 |
| 331 | 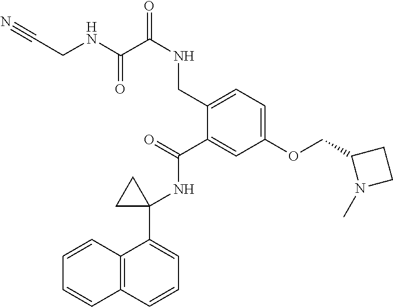 | 526.41 |
| 332 | 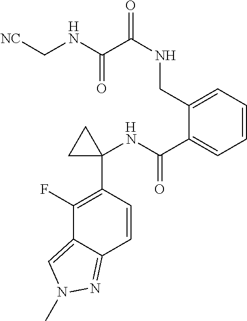 | 449.29 |
| 333 | 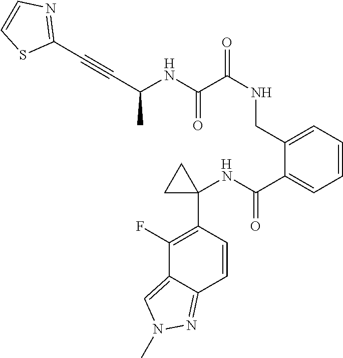 | 545.26 |
| 334 | 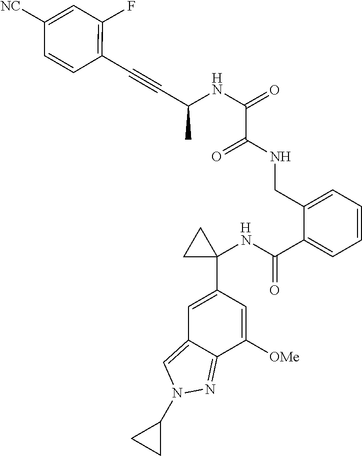 | 619.3 |
| 335 | 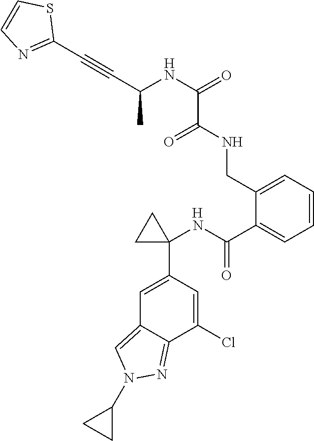 | 587.3 |
| 336 | 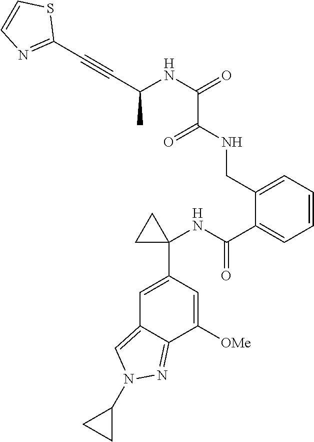 | 583.1 |
| 337 | 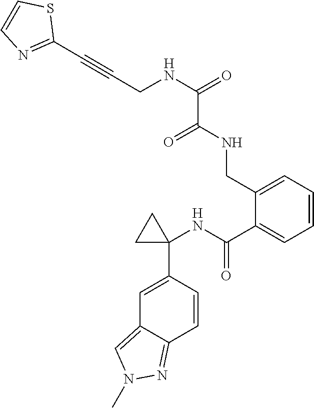 | 513.17 |
| 338 | 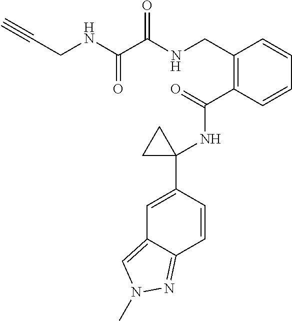 | 430.19 |
| 339 | 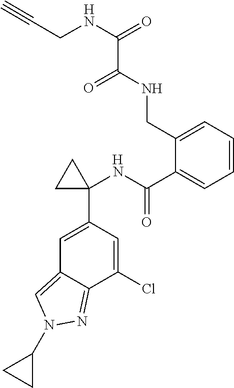 | 490.2 |
| 340 | 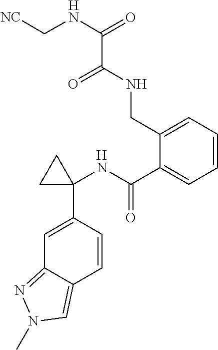 | 431.2 |
| 341 | 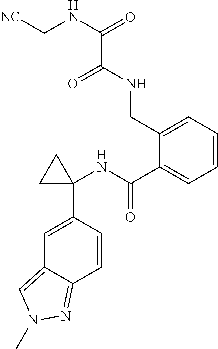 | 431.4 |
| 342 | 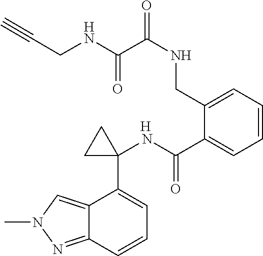 | 430.3 |
| 343 | 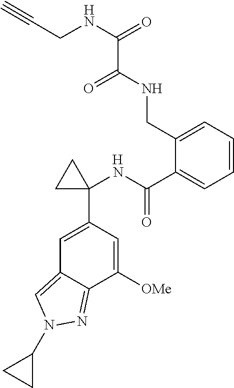 | 486.2 |
| 344 | 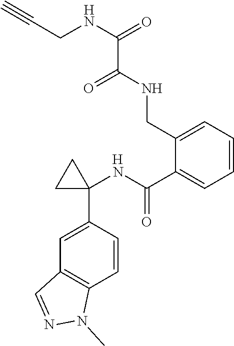 | 430.3 |
| 345 | 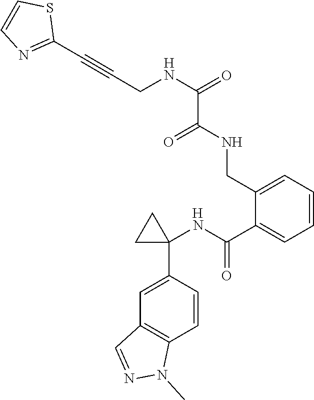 | 513.2 |
| 346 | 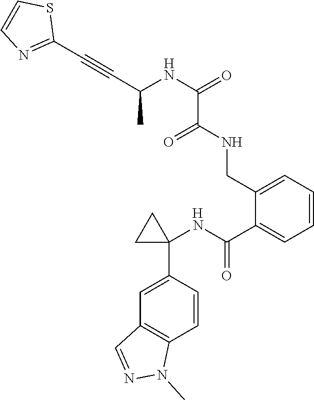 | 527.3 |
| 347 | 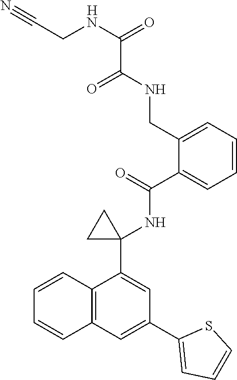 | 509.16 |
| 348 | 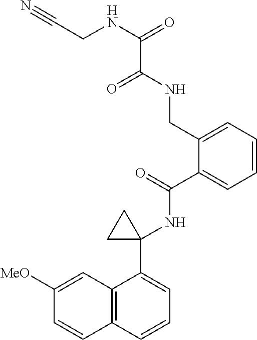 | 457.19 |
| 349 | 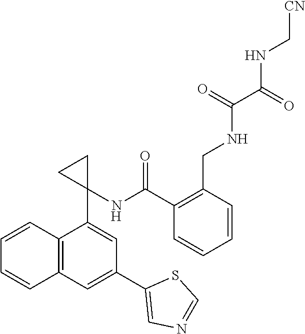 | 510.15 |
| 350 | 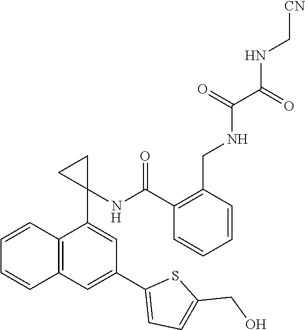 | 537.16 |
| 351 | 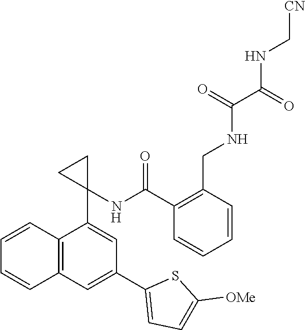 | 539.21 |
| 352 | 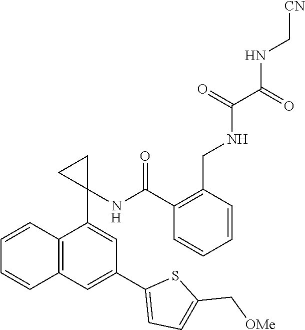 | 575.17 [M + Na]⁺ |
| 353 | 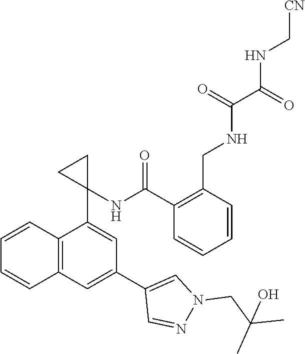 [SMILES: CC(C)(O)Cn1cc(-c2cc(C3(NC(=O)c4ccccc4CNC(=O)C(=O)NCC#N)CC3)c3ccccc3c2)cn1] | 565.24 |
| 354 | 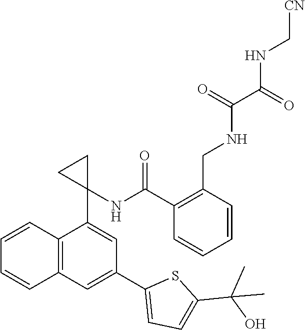 [SMILES: CC(C)(O)c1ccc(-c2cc(C3(NC(=O)c4ccccc4CNC(=O)C(=O)NCC#N)CC3)c3ccccc3c2)s1] | 589.19 [M + Na]⁺ |
| 355 | 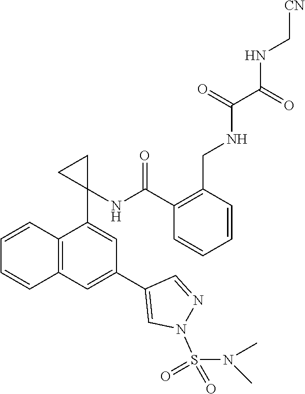 [SMILES: CN(C)S(=O)(=O)n1cc(-c2cc(C3(NC(=O)c4ccccc4CNC(=O)C(=O)NCC#N)CC3)c3ccccc3c2)cn1] | 600.21 |
| 356 | 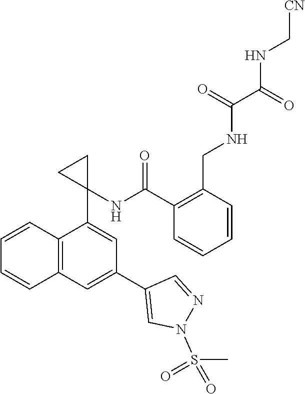 [SMILES: CS(=O)(=O)n1cc(-c2cc(C3(NC(=O)c4ccccc4CNC(=O)C(=O)NCC#N)CC3)c3ccccc3c2)cn1] | 571.18 |
| 357 | 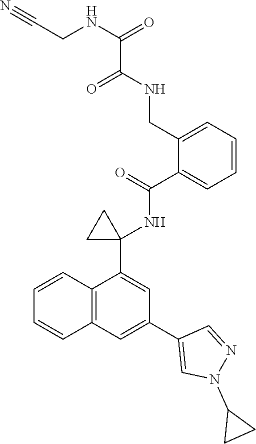 [SMILES: N#CCNC(=O)C(=O)NCc1ccccc1C(=O)NC1(c2cc(-c3cnn(C4CC4)c3)cc3ccccc23)CC1] | 533.25 |
| 358 | 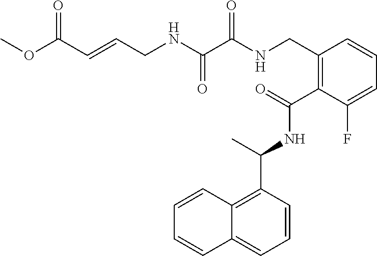 [SMILES: COC(=O)/C=C/CNC(=O)C(=O)NCc1cccc(F)c1C(=O)N[C@H](C)c1cccc2ccccc12] | 492.33 |
| 359 | 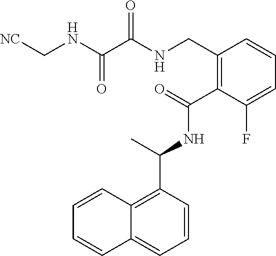 [SMILES: C[C@@H](NC(=O)c1c(F)cccc1CNC(=O)C(=O)NCC#N)c1cccc2ccccc12] | 433.27 |
| 360 | 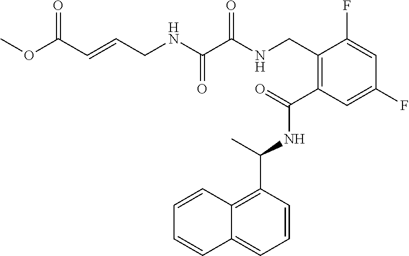 [SMILES: COC(=O)/C=C/CNC(=O)C(=O)NCc1c(F)cc(F)cc1C(=O)N[C@H](C)c1cccc2ccccc12] | 510.37 |
| 361 | 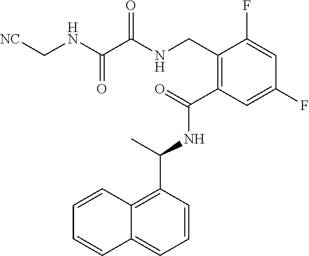 [SMILES: C[C@@H](NC(=O)c1cc(F)cc(F)c1CNC(=O)C(=O)NCC#N)c1cccc2ccccc12] | 451.29 |
| 362 | 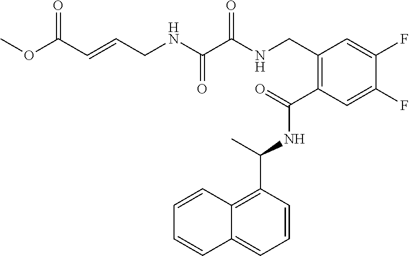 [SMILES: COC(=O)/C=C/CNC(=O)C(=O)NCc1cc(F)c(F)cc1C(=O)N[C@H](C)c1cccc2ccccc12] | 510.29 |
| 363 | 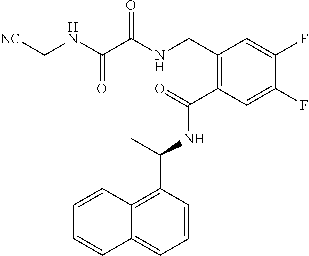 [SMILES: C[C@@H](NC(=O)c1cc(F)c(F)cc1CNC(=O)C(=O)NCC#N)c1cccc2ccccc12] | 451.24 |
| 364 | 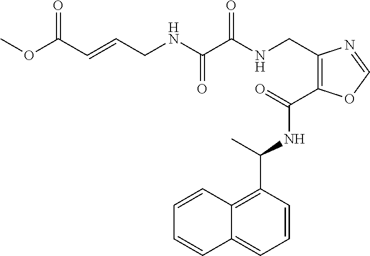 [SMILES: COC(=O)/C=C/CNC(=O)C(=O)NCc1ncoc1C(=O)N[C@H](C)c1cccc2ccccc12] | 465.26 |
| 365 | 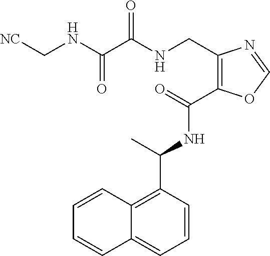 [SMILES: C[C@@H](NC(=O)c1ocnc1CNC(=O)C(=O)NCC#N)c1cccc2ccccc12] | 406.17 |
| 366 | 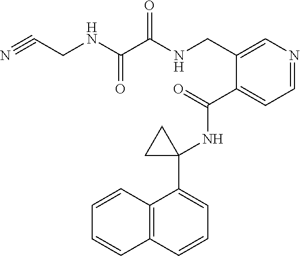 [SMILES: N#CCNC(=O)C(=O)NCc1cnccc1C(=O)NC1(c2cccc3ccccc23)CC1] | 428.30 |
| 367 | 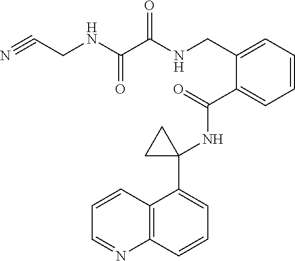 [SMILES: N#CCNC(=O)C(=O)NCc1ccccc1C(=O)NC1(c2cccc3ncccc23)CC1] | 482.24 |
| 368 | 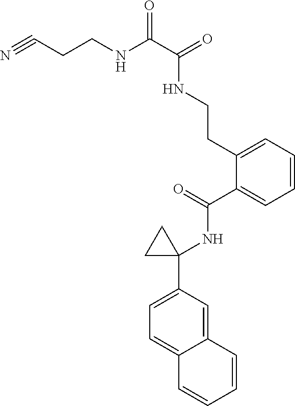 [SMILES: N#CCCNC(=O)C(=O)NCCc1ccccc1C(=O)NC1(c2ccc3ccccc3c2)CC1] | 455.23 |
| 369 | 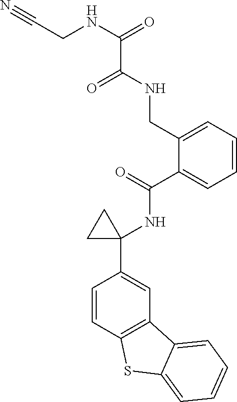 [SMILES: N#CCNC(=O)C(=O)NCc1ccccc1C(=O)NC1(c2ccc3sc4ccccc4c3c2)CC1] | 483.21 |
| 370 | 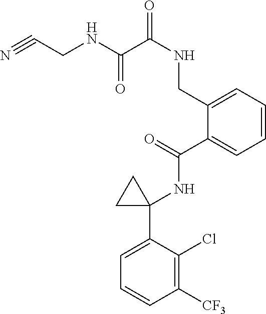 [SMILES: N#CCNC(=O)C(=O)NCc1ccccc1C(=O)NC1(c2cccc(C(F)(F)F)c2Cl)CC1] | 479.12 |
| 371 | 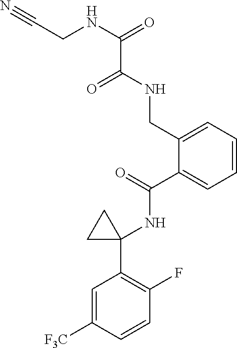 [SMILES: N#CCNC(=O)C(=O)NCc1ccccc1C(=O)NC1(c2cc(C(F)(F)F)ccc2F)CC1] | 463.13 |
| 372 | 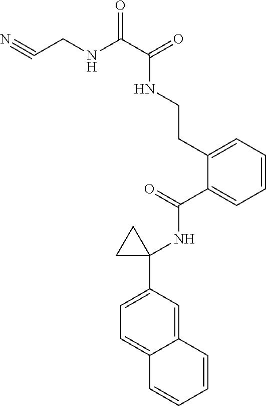 [SMILES: N#CCNC(=O)C(=O)NCCc1ccccc1C(=O)NC1(c2ccc3ccccc3c2)CC1] | 441.22 |
| 373 | 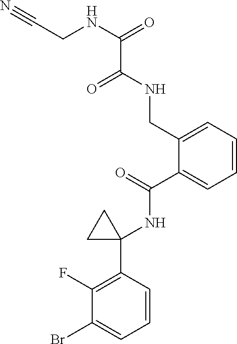 [SMILES: N#CCNC(=O)C(=O)NCc1ccccc1C(=O)NC1(c2cccc(Br)c2F)CC1] | 473.03 |
| 374 | 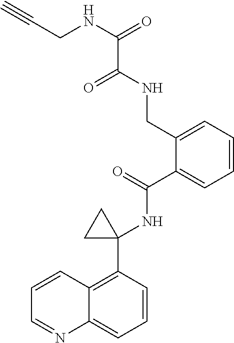 [SMILES: C#CCNC(=O)C(=O)NCc1ccccc1C(=O)NC1(c2cccc3ncccc23)CC1] | 427.14 |
| 375 | 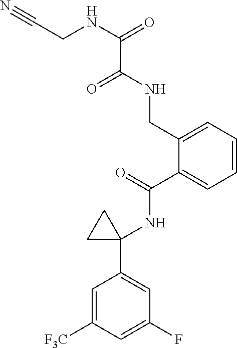 [SMILES: N#CCNC(=O)C(=O)NCc1ccccc1C(=O)NC1(c2cc(F)cc(C(F)(F)F)c2)CC1] | 463.07 |
| 376 | 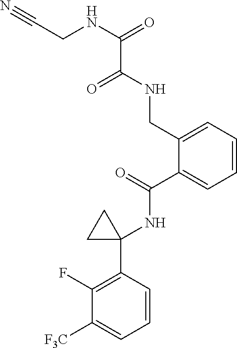 [SMILES: N#CCNC(=O)C(=O)NCc1ccccc1C(=O)NC1(c2cccc(C(F)(F)F)c2F)CC1] | 463.08 |
| 377 | 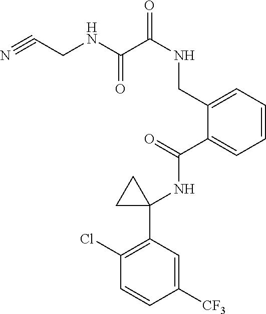 [SMILES: N#CCNC(=O)C(=O)NCc1ccccc1C(=O)NC1(c2cc(C(F)(F)F)ccc2Cl)CC1] | 479.04 |
| 378 | 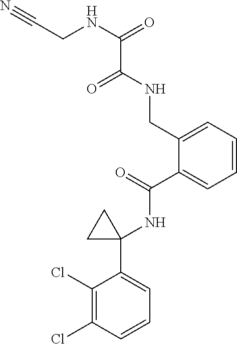 [SMILES: N#CCNC(=O)C(=O)NCc1ccccc1C(=O)NC1(c2cccc(Cl)c2Cl)CC1] | 445.02 |
| 379 | 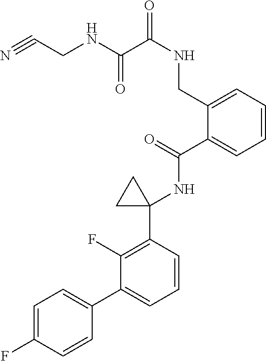 [SMILES: N#CCNC(=O)C(=O)NCc1ccccc1C(=O)NC1(c2cccc(-c3ccc(F)cc3)c2F)CC1] | 489.18 |
| 380 | 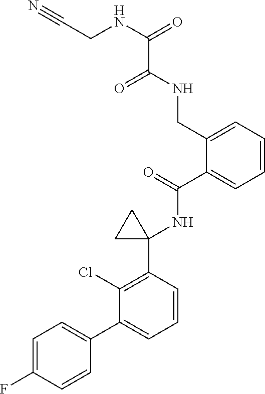 [SMILES: N#CCNC(=O)C(=O)NCc1ccccc1C(=O)NC1(c2cccc(-c3ccc(F)cc3)c2Cl)CC1] | 505.21 |
| 381 | 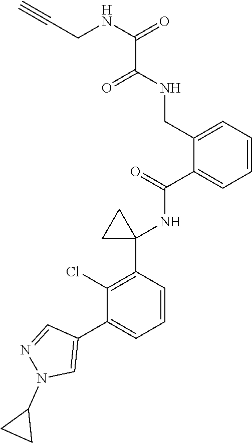 [SMILES: C#CCNC(=O)C(=O)NCc1ccccc1C(=O)NC1(c2cccc(-c3cnn(C4CC4)c3)c2Cl)CC1] | 516.18 |
| 382 | 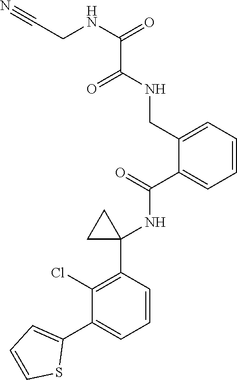 [SMILES: N#CCNC(=O)C(=O)NCc1ccccc1C(=O)NC1(c2cccc(-c3cccs3)c2Cl)CC1] | 493.19 |
| 383 | 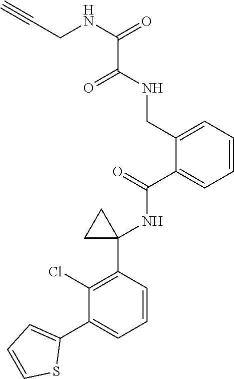 [SMILES: C#CCNC(=O)C(=O)NCc1ccccc1C(=O)NC1(c2cccc(-c3cccs3)c2Cl)CC1] | 492.18 |
| 384 | 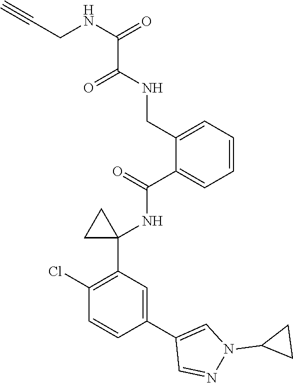 [SMILES: C#CCNC(=O)C(=O)NCc1ccccc1C(=O)NC1(c2cc(-c3cnn(C4CC4)c3)ccc2Cl)CC1] | 516.23 |
| 385 | 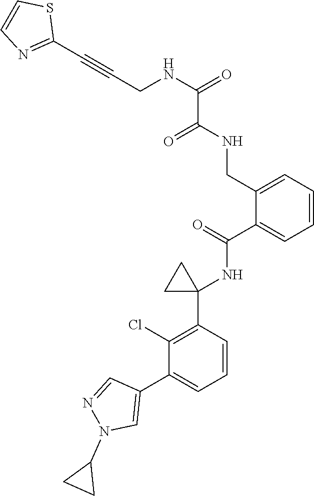 [SMILES: O=C(NCC#Cc1nccs1)C(=O)NCc1ccccc1C(=O)NC1(c2cccc(-c3cnn(C4CC4)c3)c2Cl)CC1] | 599.18 |
| 386 | 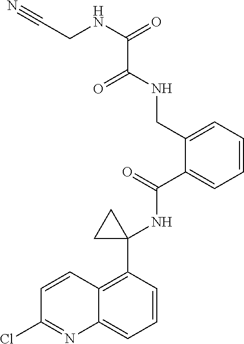 [SMILES: N#CCNC(=O)C(=O)NCc1ccccc1C(=O)NC1(c2cccc3nc(Cl)ccc23)CC1] | 462.15 |
| 387 | 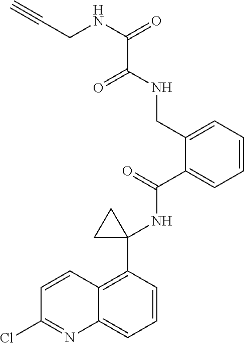 [SMILES: C#CCNC(=O)C(=O)NCc1ccccc1C(=O)NC1(c2cccc3nc(Cl)ccc23)CC1] | 461.16 |
| 388 | 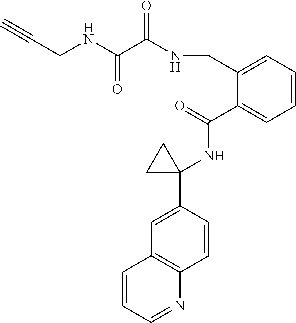 [SMILES: C#CCNC(=O)C(=O)NCc1ccccc1C(=O)NC1(c2ccc3ncccc3c2)CC1] | 427.34 |
| 389 | 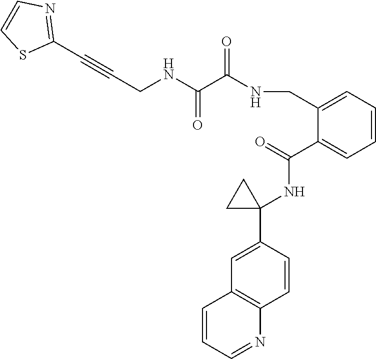 [SMILES: O=C(NCC#Cc1nccs1)C(=O)NCc1ccccc1C(=O)NC1(c2ccc3ncccc3c2)CC1] | 510.11 |
| 390 | 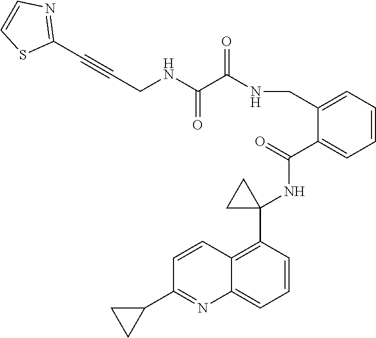 [SMILES: O=C(NCC#Cc1nccs1)C(=O)NCc1ccccc1C(=O)NC1(c2cccc3nc(C4CC4)ccc23)CC1] | 550.31 |
| 391 | 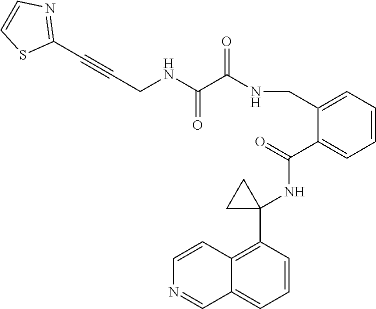 [SMILES: O=C(NCC#Cc1nccs1)C(=O)NCc1ccccc1C(=O)NC1(c2cccc3cnccc23)CC1] | 510.15 |
| 392 | 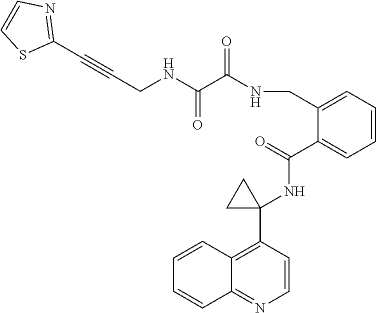 [SMILES: O=C(NCC#Cc1nccs1)C(=O)NCc1ccccc1C(=O)NC1(c2ccnc3ccccc23)CC1] | 510.15 |
| 393 | 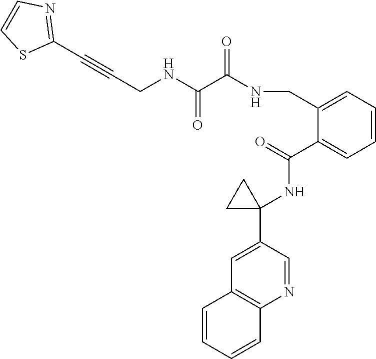 [SMILES: O=C(NCC#Cc1nccs1)C(=O)NCc1ccccc1C(=O)NC1(c2cnc3ccccc3c2)CC1] | 510.21 |
| 394 | 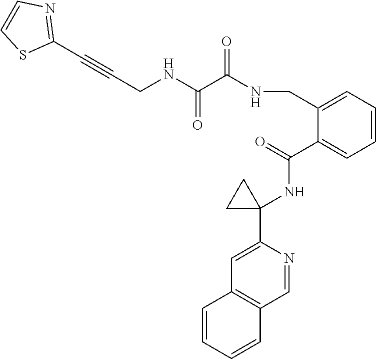 [SMILES: O=C(NCC#Cc1nccs1)C(=O)NCc1ccccc1C(=O)NC1(c2cc3ccccc3cn2)CC1] | 510.20 |
| 395 | 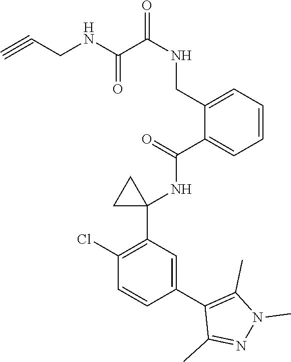 [SMILES: C#CCNC(=O)C(=O)NCc1ccccc1C(=O)NC1(c2cc(-c3c(C)nn(C)c3C)ccc2Cl)CC1] | 518.39 |
| 396 | 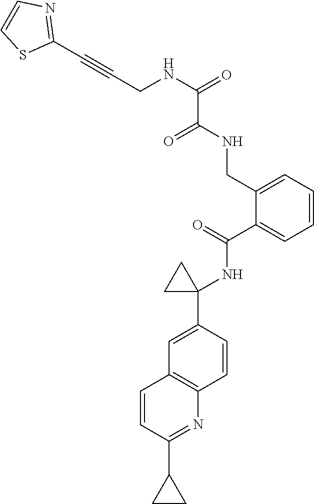 [SMILES: O=C(NCC#Cc1nccs1)C(=O)NCc1ccccc1C(=O)NC1(c2ccc3nc(C4CC4)ccc3c2)CC1] | 550.23 |
| 397 | 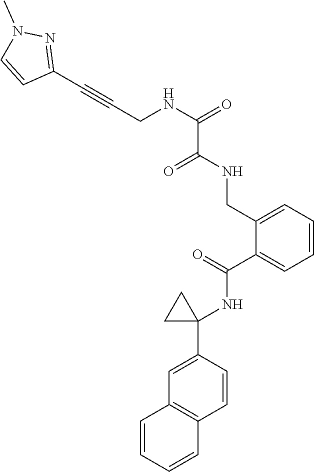 [SMILES: Cn1ccc(C#CCNC(=O)C(=O)NCc2ccccc2C(=O)NC2(c3ccc4ccccc4c3)CC2)n1] | 506.21 |
| 398 | 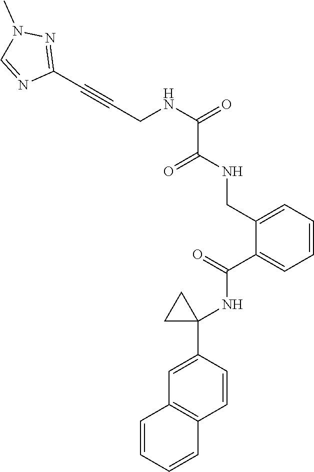 [SMILES: Cn1cnc(C#CCNC(=O)C(=O)NCc2ccccc2C(=O)NC2(c3ccc4ccccc4c3)CC2)n1] | 507.18 |
| 399 | 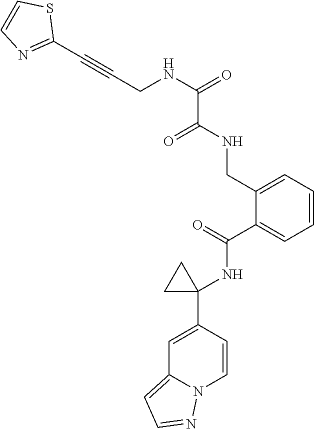 [SMILES: O=C(NCC#Cc1nccs1)C(=O)NCc1ccccc1C(=O)NC1(c2ccn3nccc3c2)CC1] | 499.13 |
| 400 | 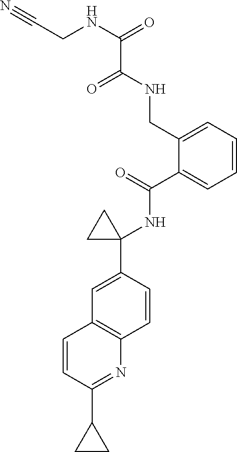 [SMILES: N#CCNC(=O)C(=O)NCc1ccccc1C(=O)NC1(c2ccc3nc(C4CC4)ccc3c2)CC1] | 468.2 |
| 401 | 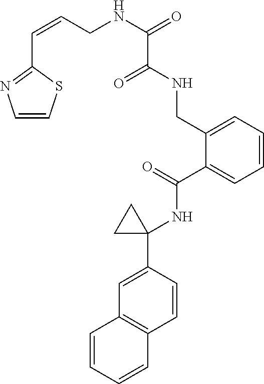 [SMILES: O=C(NC/C=C\c1nccs1)C(=O)NCc1ccccc1C(=O)NC1(c2ccc3ccccc3c2)CC1] | 511.25 |
| 402 | 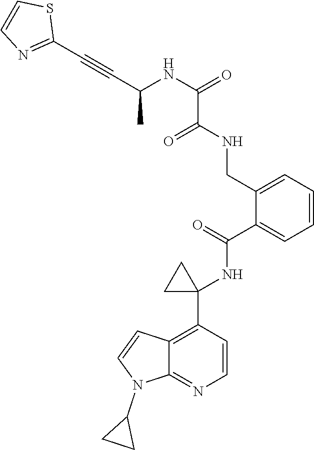 [SMILES: C[C@@H](C#Cc1nccs1)NC(=O)C(=O)NCc1ccccc1C(=O)NC1(c2ccnc3c2ccn3C2CC2)CC1] | 553.33 |
| 403 | 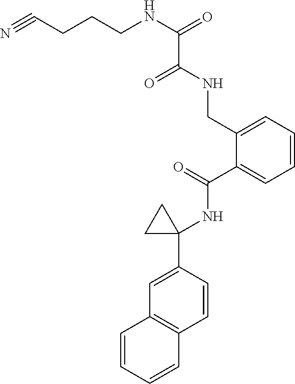 [SMILES: N#CCCCNC(=O)C(=O)NCc1ccccc1C(=O)NC1(c2ccc3ccccc3c2)CC1] | 455.23 |
| 404 | 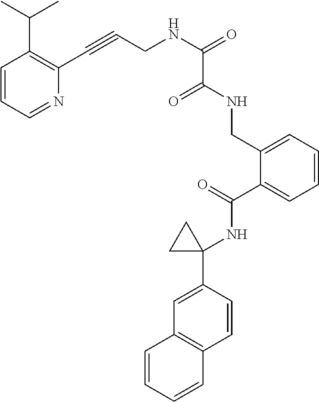 [SMILES: CC(C)c1cccnc1C#CCNC(=O)C(=O)NCc1ccccc1C(=O)NC1(c2ccc3ccccc3c2)CC1] | 545.42 |
| 405 | 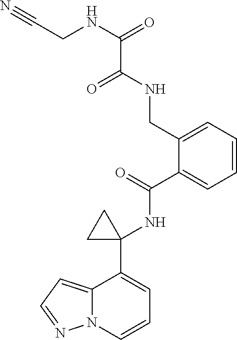 [SMILES: N#CCNC(=O)C(=O)NCc1ccccc1C(=O)NC1(c2cccn3nccc23)CC1] | 417.18 |
| 406 | 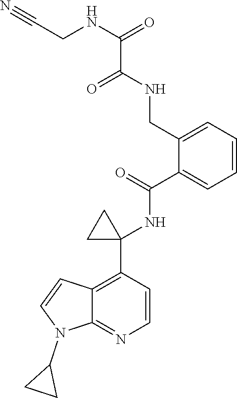 [SMILES: N#CCNC(=O)C(=O)NCc1ccccc1C(=O)NC1(c2ccnc3c2ccn3C2CC2)CC1] | 457.36 |
| 407 | 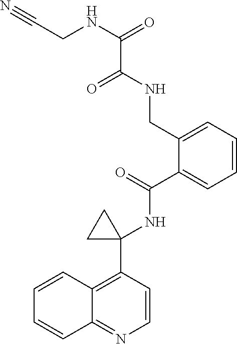 [SMILES: N#CCNC(=O)C(=O)NCc1ccccc1C(=O)NC1(c2ccnc3ccccc23)CC1] | 428.15 |
| 408 | 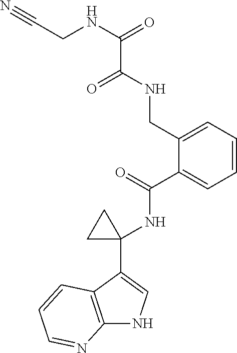 [SMILES: N#CCNC(=O)C(=O)NCc1ccccc1C(=O)NC1(c2c[nH]c3ncccc23)CC1] | 417.27 |
| 409 | 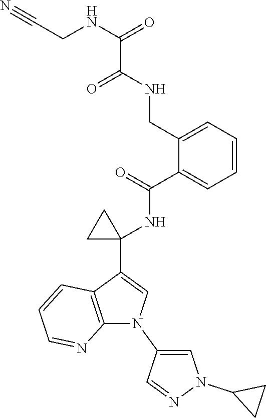 [SMILES: N#CCNC(=O)C(=O)NCc1ccccc1C(=O)NC1(c2cn(-c3cnn(C4CC4)c3)c3ncccc23)CC1] | 523.25 |
| 410 | 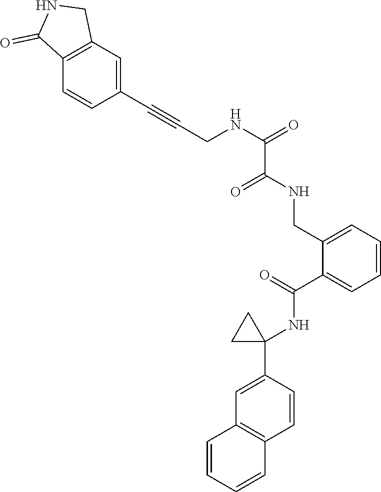 [SMILES: O=C(NCC#Cc1ccc2c(c1)CNC2=O)C(=O)NCc1ccccc1C(=O)NC1(c2ccc3ccccc3c2)CC1] | 557.35 |
| 411 | 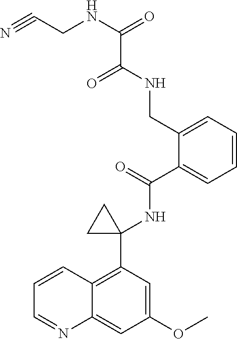 [SMILES: COc1cc(C2(NC(=O)c3ccccc3CNC(=O)C(=O)NCC#N)CC2)c2cccnc2c1] | 458.24 |
| 412 | 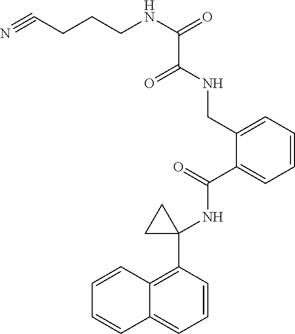 [SMILES: N#CCCCNC(=O)C(=O)NCc1ccccc1C(=O)NC1(c2cccc3ccccc23)CC1] | 455.23 |
| 413 | 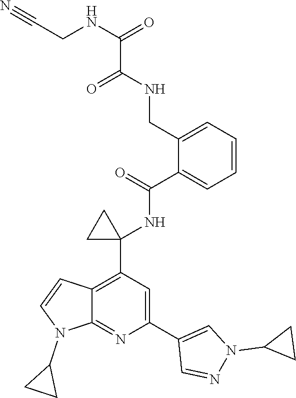 [SMILES: N#CCNC(=O)C(=O)NCc1ccccc1C(=O)NC1(c2cc(-c3cnn(C4CC4)c3)nc3c2ccn3C2CC2)CC1] | 563.32 |
| 414 | 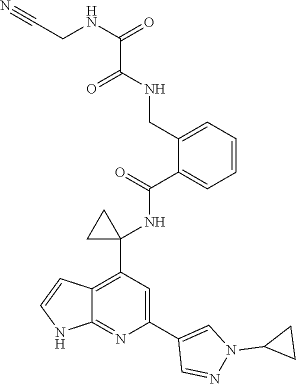 [SMILES: N#CCNC(=O)C(=O)NCc1ccccc1C(=O)NC1(c2cc(-c3cnn(C4CC4)c3)nc3[nH]ccc23)CC1] | 523.26 |
| 415 | 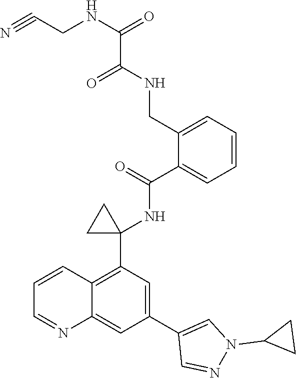 [SMILES: N#CCNC(=O)C(=O)NCc1ccccc1C(=O)NC1(c2cc(-c3cnn(C4CC4)c3)cc3ncccc23)CC1] | 534.32 |
| 416 | 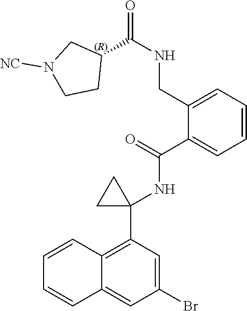 | 517.09 |
| 417 | 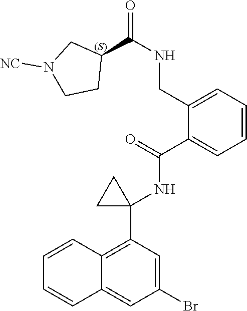 | 517.11 |
| 418 | 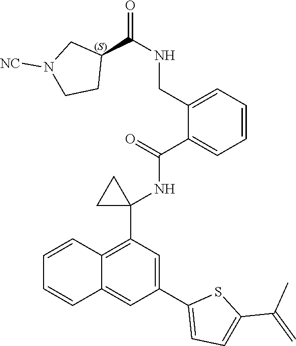 | 561.40 |
| 419 | 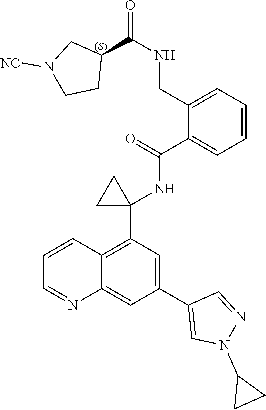 | 546.36 |
| 420 | 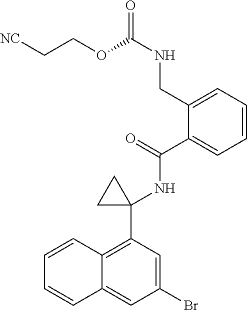 | 492.09 |
| 421 | 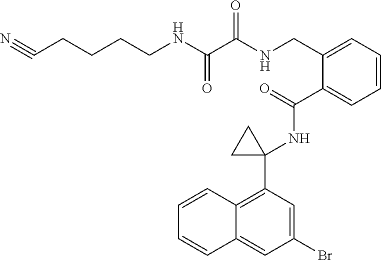 [SMILES: N#CCCCCNC(=O)C(=O)NCc1ccccc1C(=O)NC1(c2cc(Br)cc3ccccc23)CC1] | 547.16 |
| 422 | 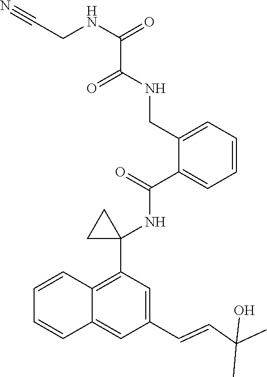 [SMILES: CC(C)(O)/C=C/c1cc(C2(NC(=O)c3ccccc3CNC(=O)C(=O)NCC#N)CC2)c2ccccc2c1] | 493.30 (M + H⁺ − H₂O) |
| 423 | 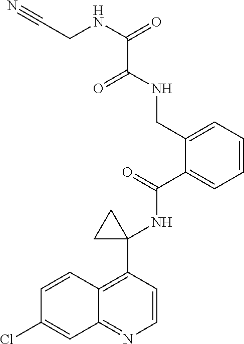 [SMILES: N#CCNC(=O)C(=O)NCc1ccccc1C(=O)NC1(c2ccnc3cc(Cl)ccc23)CC1] | 462.26 |
| 424 | 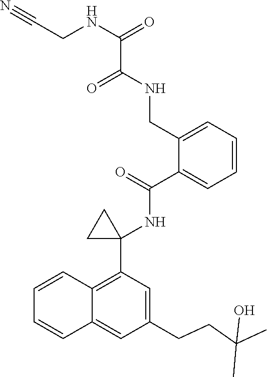 [SMILES: CC(C)(O)CCc1cc(C2(NC(=O)c3ccccc3CNC(=O)C(=O)NCC#N)CC2)c2ccccc2c1] | 511.40 (M − H)⁻ |
| 425 | 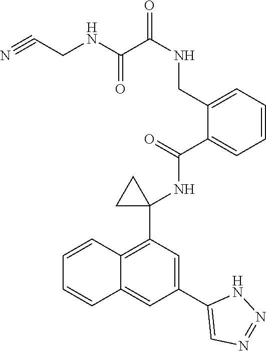 [SMILES: N#CCNC(=O)C(=O)NCc1ccccc1C(=O)NC1(c2cc(-c3cnn[nH]3)cc3ccccc23)CC1] | 494.24 |
| 426 | 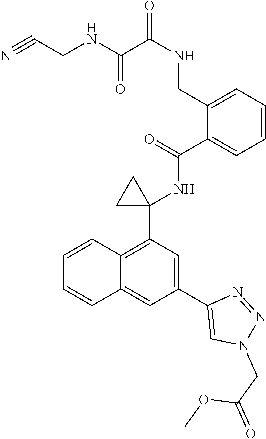 [SMILES: COC(=O)Cn1cc(-c2cc(C3(NC(=O)c4ccccc4CNC(=O)C(=O)NCC#N)CC3)c3ccccc3c2)nn1] | 566.40 |
| 427 | 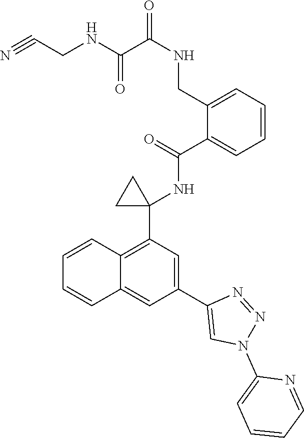 [SMILES: N#CCNC(=O)C(=O)NCc1ccccc1C(=O)NC1(c2cc(-c3cn(-c4ccccn4)nn3)cc3ccccc23)CC1] | 571.42 |
| 428 | 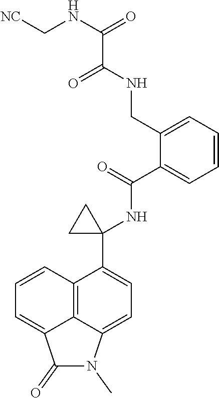 [SMILES: CN1C(=O)c2cccc3c(C4(NC(=O)c5ccccc5CNC(=O)C(=O)NCC#N)CC4)ccc1c23] | 482.18 |
| 429 | 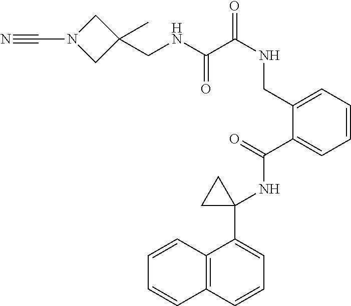 [SMILES: CC1(CNC(=O)C(=O)NCc2ccccc2C(=O)NC2(c3cccc4ccccc34)CC2)CN(C#N)C1] | 496.24 |
| 430 | 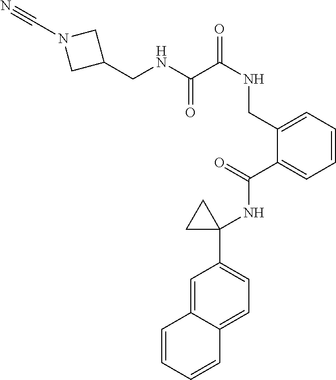 [SMILES: N#CN1CC(CNC(=O)C(=O)NCc2ccccc2C(=O)NC2(c3ccc4ccccc4c3)CC2)C1] | 482.22 |
| 431 | 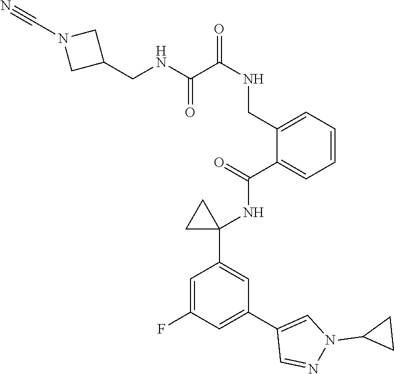 [SMILES: N#CN1CC(CNC(=O)C(=O)NCc2ccccc2C(=O)NC2(c3cc(F)cc(-c4cnn(C5CC5)c4)c3)CC2)C1] | 556.25 |
| 432 | 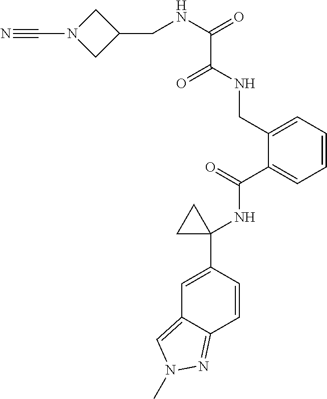 [SMILES: Cn1cc2cc(C3(NC(=O)c4ccccc4CNC(=O)C(=O)NCC4CN(C#N)C4)CC3)ccc2n1] | 486.23 |
| 433 | 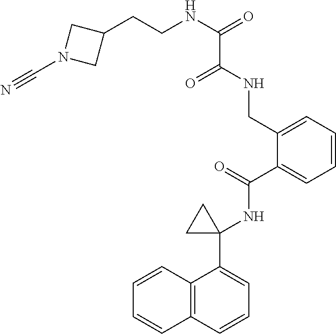 [SMILES: N#CN1CC(CCNC(=O)C(=O)NCc2ccccc2C(=O)NC2(c3cccc4ccccc34)CC2)C1] | 494.22 (M − H)⁻ |
| 434 | 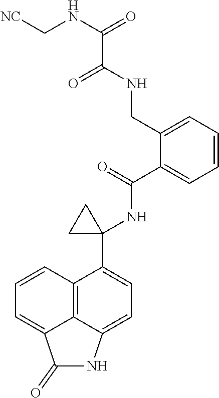 [SMILES: N#CCNC(=O)C(=O)NCc1ccccc1C(=O)NC1(c2ccc3c4c(cccc24)C(=O)N3)CC1] | 466.15 (M − H)⁻ |
| 435 | 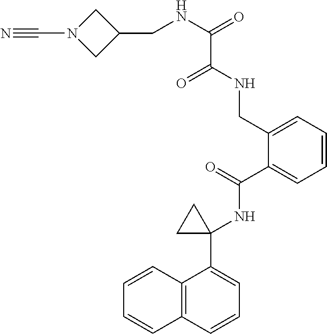 [SMILES: N#CN1CC(CNC(=O)C(=O)NCc2ccccc2C(=O)NC2(c3cccc4ccccc34)CC2)C1] | 480.20 (M − H)⁻ |
| 437 | 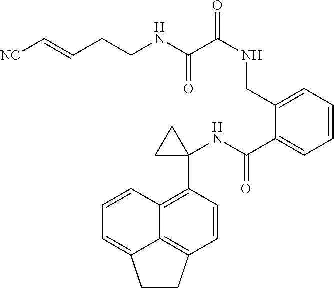 [SMILES: N#C/C=C/CCNC(=O)C(=O)NCc1ccccc1C(=O)NC1(c2ccc3c4c(cccc24)CC3)CC1] | 491.21 (M − H)⁻ |
| 438 | 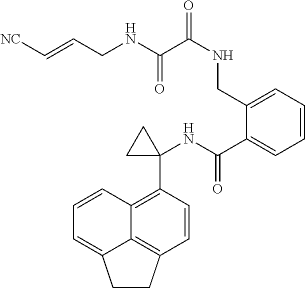 [SMILES: N#C/C=C/CNC(=O)C(=O)NCc1ccccc1C(=O)NC1(c2ccc3c4c(cccc24)CC3)CC1] | 477.19 (M − H)⁻ |
| 439 | 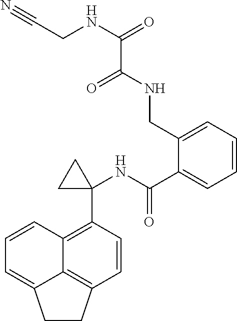 [SMILES: N#CCNC(=O)C(=O)NCc1ccccc1C(=O)NC1(c2ccc3c4c(cccc24)CC3)CC1] | 451.23 (M − H)⁻ |
| 440 | 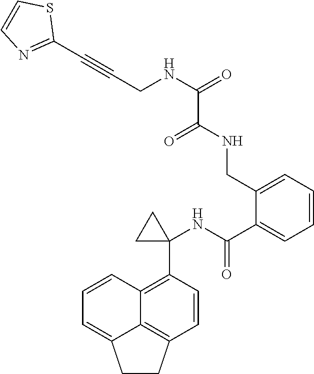 [SMILES: O=C(NCC#Cc1nccs1)C(=O)NCc1ccccc1C(=O)NC1(c2ccc3c4c(cccc24)CC3)CC1] | 533.17 (M − H)⁻ |
| 442 | 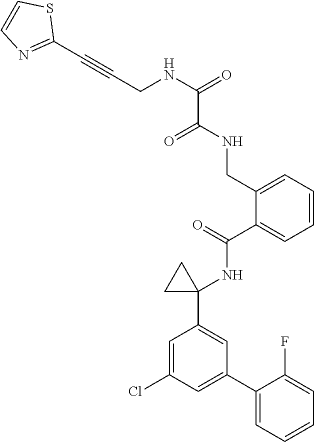 [SMILES: O=C(NCC#Cc1nccs1)C(=O)NCc1ccccc1C(=O)NC1(c2cc(Cl)cc(-c3ccccc3F)c2)CC1] | 585.12 (M − H)⁻ |
| 443 | 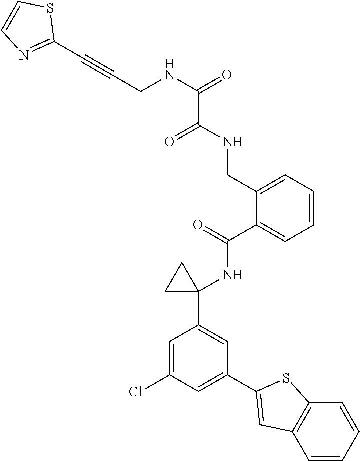 [SMILES: O=C(NCC#Cc1nccs1)C(=O)NCc1ccccc1C(=O)NC1(c2cc(Cl)cc(-c3cc4ccccc4s3)c2)CC1] | 623.10 (M − H)⁻ |
| 444 | 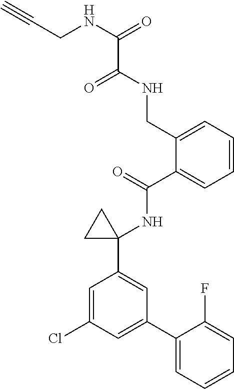 [SMILES: C#CCNC(=O)C(=O)NCc1ccccc1C(=O)NC1(c2cc(Cl)cc(-c3ccccc3F)c2)CC1] | 502.13 (M − H)⁻ |
| 445 | 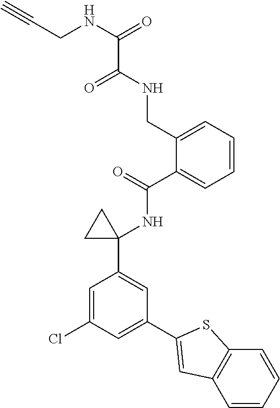 [SMILES: C#CCNC(=O)C(=O)NCc1ccccc1C(=O)NC1(c2cc(Cl)cc(-c3cc4ccccc4s3)c2)CC1] | 540.12 (M − H)⁻ |
| 446 | 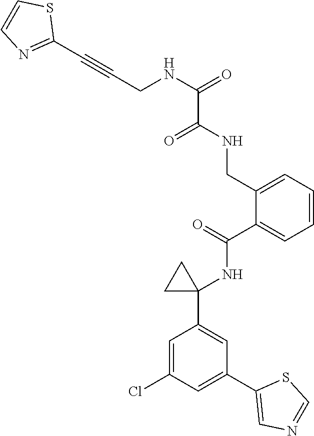 [SMILES: O=C(NCC#Cc1nccs1)C(=O)NCc1ccccc1C(=O)NC1(c2cc(Cl)cc(-c3cncs3)c2)CC1] | 574.08 (M − H)⁻ |
| 447 | 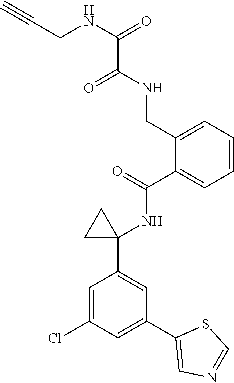 [SMILES: C#CCNC(=O)C(=O)NCc1ccccc1C(=O)NC1(c2cc(Cl)cc(-c3cncs3)c2)CC1] | 491.09 (M − H)⁻ |
| 448 | 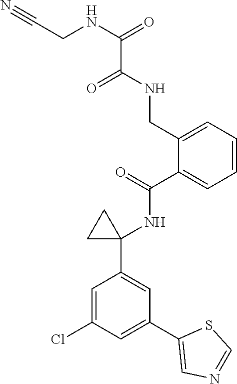 [SMILES: N#CCNC(=O)C(=O)NCc1ccccc1C(=O)NC1(c2cc(Cl)cc(-c3cncs3)c2)CC1] | 492.09 (M − H)⁻ |
| 449 | 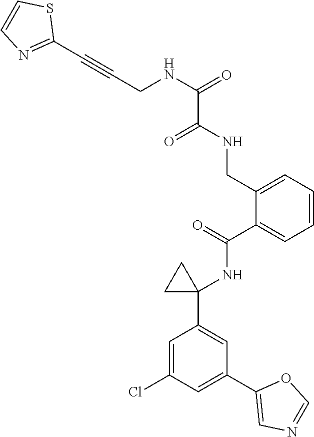 [SMILES: O=C(NCC#Cc1nccs1)C(=O)NCc1ccccc1C(=O)NC1(c2cc(Cl)cc(-c3cnco3)c2)CC1] | 560.12 |
| 450 | 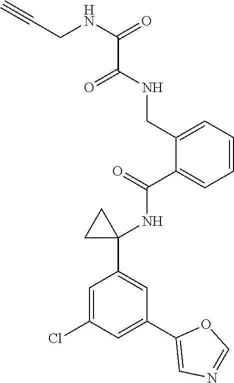 [SMILES: C#CCNC(=O)C(=O)NCc1ccccc1C(=O)NC1(c2cc(Cl)cc(-c3cnco3)c2)CC1] | 477.13 |
| 451 | 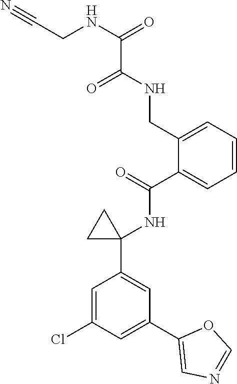 [SMILES: N#CCNC(=O)C(=O)NCc1ccccc1C(=O)NC1(c2cc(Cl)cc(-c3cnco3)c2)CC1] | 478.13 |
| 452 | 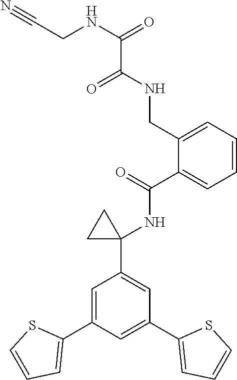 [SMILES: N#CCNC(=O)C(=O)NCc1ccccc1C(=O)NC1(c2cc(-c3cccs3)cc(-c3cccs3)c2)CC1] | 539.12 (M − H)⁻ |
| 453 | 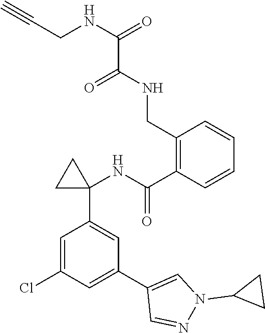 [SMILES: C#CCNC(=O)C(=O)NCc1ccccc1C(=O)NC1(c2cc(Cl)cc(-c3cnn(C4CC4)c3)c2)CC1] | 514.35 (M − H)⁻ |
| 454 | 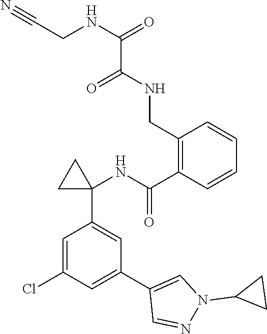 [SMILES: N#CCNC(=O)C(=O)NCc1ccccc1C(=O)NC1(c2cc(Cl)cc(-c3cnn(C4CC4)c3)c2)CC1] | 515.33 (M − H)⁻ |
| 455 | 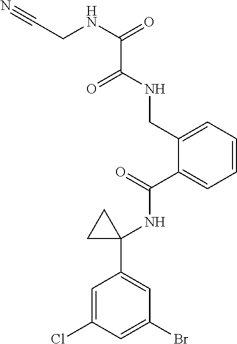 [SMILES: N#CCNC(=O)C(=O)NCc1ccccc1C(=O)NC1(c2cc(Cl)cc(Br)c2)CC1] | 487.02 (M − H)⁻ |
| 456 | 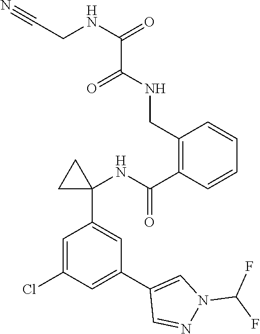 [SMILES: N#CCNC(=O)C(=O)NCc1ccccc1C(=O)NC1(c2cc(Cl)cc(-c3cnn(C(F)F)c3)c2)CC1] | 527.14 |
| 458 | 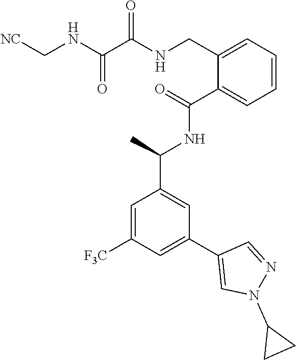 [SMILES: C[C@@H](NC(=O)c1ccccc1CNC(=O)C(=O)NCC#N)c1cc(-c2cnn(C3CC3)c2)cc(C(F)(F)F)c1] | 539.2 |
| 459 | 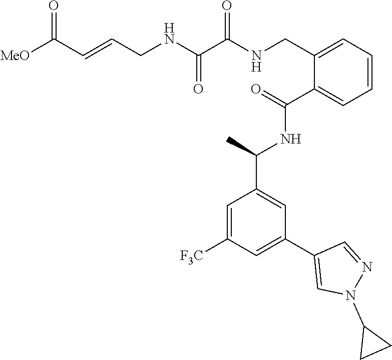 [SMILES: COC(=O)/C=C/CNC(=O)C(=O)NCc1ccccc1C(=O)N[C@H](C)c1cc(-c2cnn(C3CC3)c2)cc(C(F)(F)F)c1] | 598.2 |
| 460 | 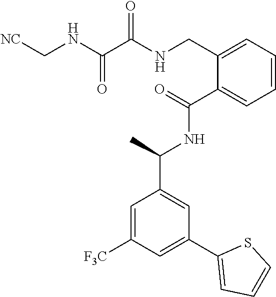 [SMILES: C[C@@H](NC(=O)c1ccccc1CNC(=O)C(=O)NCC#N)c1cc(-c2cccs2)cc(C(F)(F)F)c1] | 515.1 |
| 461 | 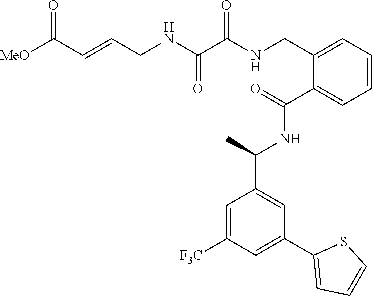 [SMILES: COC(=O)/C=C/CNC(=O)C(=O)NCc1ccccc1C(=O)N[C@H](C)c1cc(-c2cccs2)cc(C(F)(F)F)c1] | 574.1 |
| 462 | 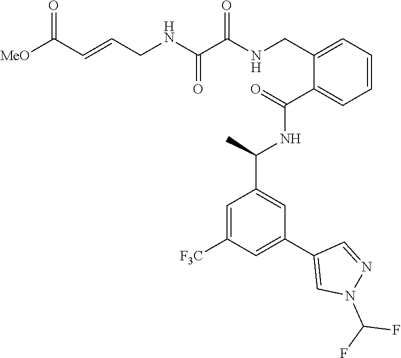 [SMILES: COC(=O)/C=C/CNC(=O)C(=O)NCc1ccccc1C(=O)N[C@H](C)c1cc(-c2cnn(C(F)F)c2)cc(C(F)(F)F)c1] | 608.1 |
| 463 | 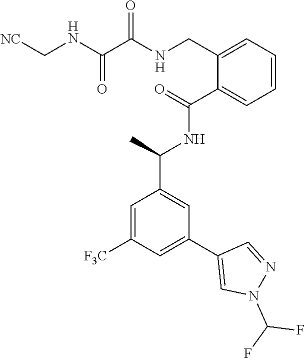 [SMILES: C[C@@H](NC(=O)c1ccccc1CNC(=O)C(=O)NCC#N)c1cc(-c2cnn(C(F)F)c2)cc(C(F)(F)F)c1] | 549.1 |
| 464 | 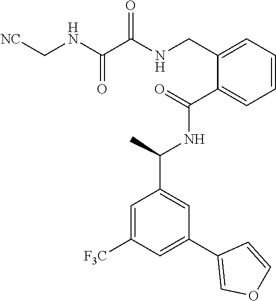 [SMILES: C[C@@H](NC(=O)c1ccccc1CNC(=O)C(=O)NCC#N)c1cc(-c2ccoc2)cc(C(F)(F)F)c1] | 499.0 |
| 465 | 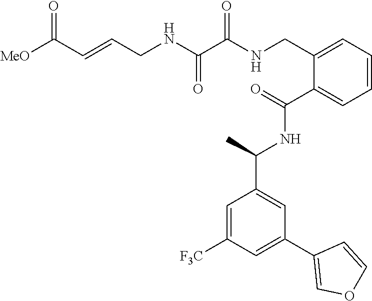 [SMILES: COC(=O)/C=C/CNC(=O)C(=O)NCc1ccccc1C(=O)N[C@H](C)c1cc(-c2ccoc2)cc(C(F)(F)F)c1] | 558.1 |
| 466 | 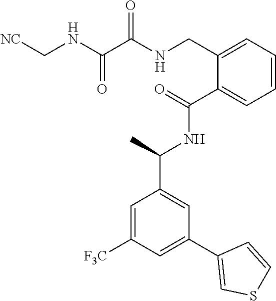 [SMILES: C[C@@H](NC(=O)c1ccccc1CNC(=O)C(=O)NCC#N)c1cc(-c2ccsc2)cc(C(F)(F)F)c1] | 515.1 |
| 467 | 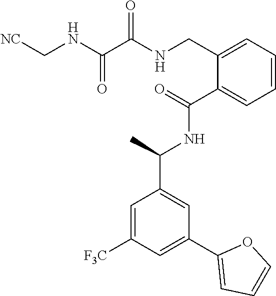 [SMILES: C[C@@H](NC(=O)c1ccccc1CNC(=O)C(=O)NCC#N)c1cc(-c2ccco2)cc(C(F)(F)F)c1] | 499.0 |
| 468 | 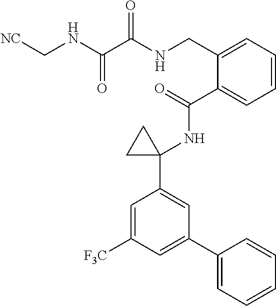 [SMILES: N#CCNC(=O)C(=O)NCc1ccccc1C(=O)NC1(c2cc(-c3ccccc3)cc(C(F)(F)F)c2)CC1] | 521.1 |
| 469 | 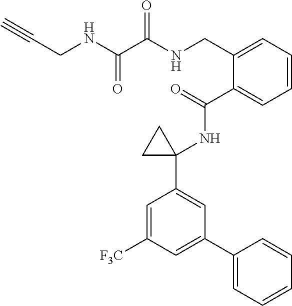 [SMILES: C#CCNC(=O)C(=O)NCc1ccccc1C(=O)NC1(c2cc(-c3ccccc3)cc(C(F)(F)F)c2)CC1] | 520.1 |
| 470 | 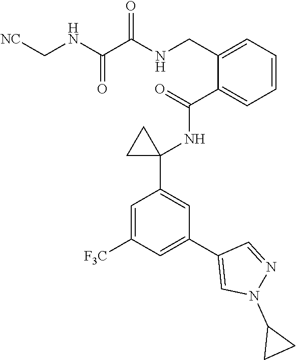 [SMILES: N#CCNC(=O)C(=O)NCc1ccccc1C(=O)NC1(c2cc(-c3cnn(C4CC4)c3)cc(C(F)(F)F)c2)CC1] | 551.1 |
| 471 | 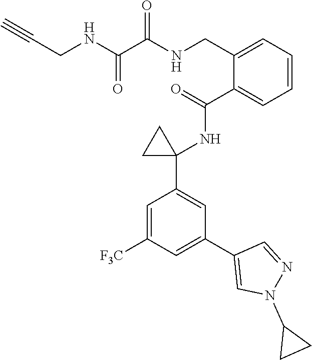 [SMILES: C#CCNC(=O)C(=O)NCc1ccccc1C(=O)NC1(c2cc(-c3cnn(C4CC4)c3)cc(C(F)(F)F)c2)CC1] | 550.0 |
| 472 | 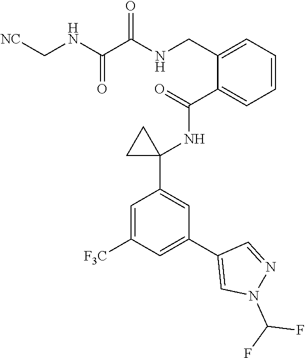 [SMILES: N#CCNC(=O)C(=O)NCc1ccccc1C(=O)NC1(c2cc(-c3cnn(C(F)F)c3)cc(C(F)(F)F)c2)CC1] | 561.1 |
| 473 | 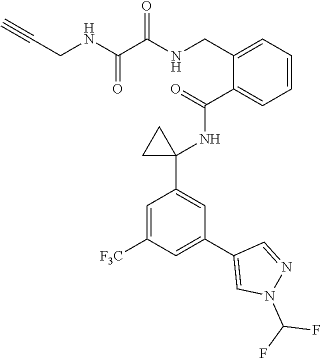 [SMILES: C#CCNC(=O)C(=O)NCc1ccccc1C(=O)NC1(c2cc(-c3cnn(C(F)F)c3)cc(C(F)(F)F)c2)CC1] | 560.1 |
| 474 | 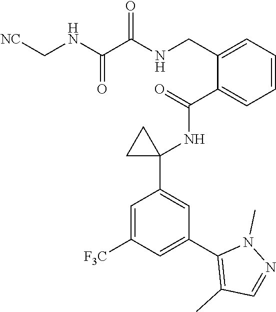 [SMILES: Cc1cnn(C)c1-c1cc(C(F)(F)F)cc(C2(NC(=O)c3ccccc3CNC(=O)C(=O)NCC#N)CC2)c1] | 539.3 |
| 475 | 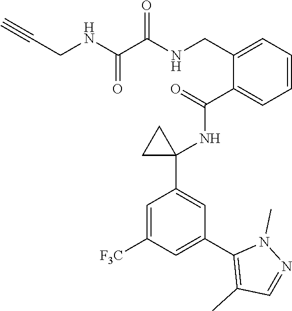 [SMILES: C#CCNC(=O)C(=O)NCc1ccccc1C(=O)NC1(c2cc(-c3c(C)cnn3C)cc(C(F)(F)F)c2)CC1] | 538.3 |
| 476 | 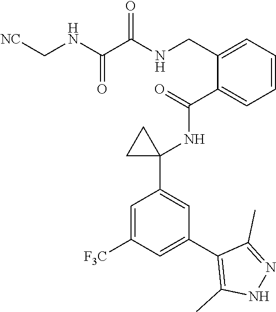 [SMILES: Cc1n[nH]c(C)c1-c1cc(C(F)(F)F)cc(C2(NC(=O)c3ccccc3CNC(=O)C(=O)NCC#N)CC2)c1] | 539.2 |
| 477 | 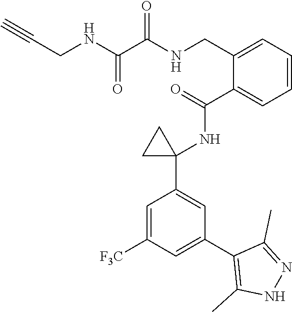 [SMILES: C#CCNC(=O)C(=O)NCc1ccccc1C(=O)NC1(c2cc(-c3c(C)n[nH]c3C)cc(C(F)(F)F)c2)CC1] | 538.3 |
| 478 | 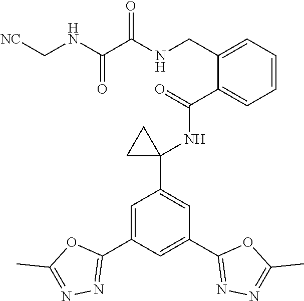 [SMILES: Cc1nnc(-c2cc(-c3nnc(C)o3)cc(C3(NC(=O)c4ccccc4CNC(=O)C(=O)NCC#N)CC3)c2)o1] | 541.1 |
| 479 | 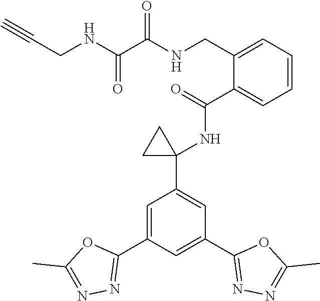 [SMILES: C#CCNC(=O)C(=O)NCc1ccccc1C(=O)NC1(c2cc(-c3nnc(C)o3)cc(-c3nnc(C)o3)c2)CC1] | 540.0 |
| 480 | 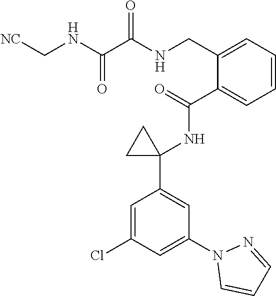 [SMILES: N#CCNC(=O)C(=O)NCc1ccccc1C(=O)NC1(c2cc(Cl)cc(-n3cccn3)c2)CC1] | 477.0 |
| 481 | 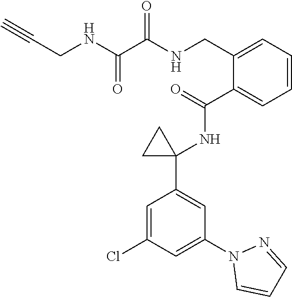 [SMILES: C#CCNC(=O)C(=O)NCc1ccccc1C(=O)NC1(c2cc(Cl)cc(-n3cccn3)c2)CC1] | 476.0 |
| 482 | 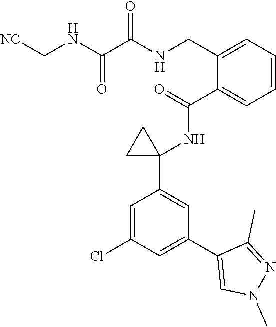 [SMILES: Cc1nn(C)cc1-c1cc(Cl)cc(C2(NC(=O)c3ccccc3CNC(=O)C(=O)NCC#N)CC2)c1] | 505.0 |
| 483 | 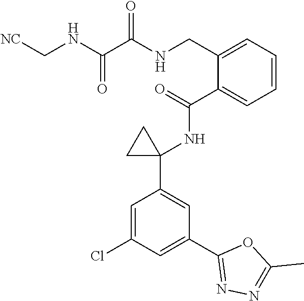 [SMILES: Cc1nnc(-c2cc(Cl)cc(C3(NC(=O)c4ccccc4CNC(=O)C(=O)NCC#N)CC3)c2)o1] | 492.9 |
| 484 | 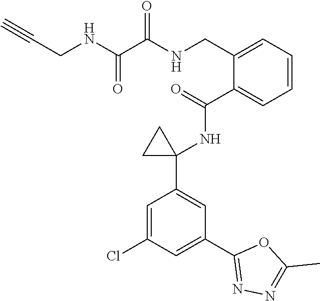 [SMILES: C#CCNC(=O)C(=O)NCc1ccccc1C(=O)NC1(c2cc(Cl)cc(-c3nnc(C)o3)c2)CC1] | 492.0 |
| 485 | 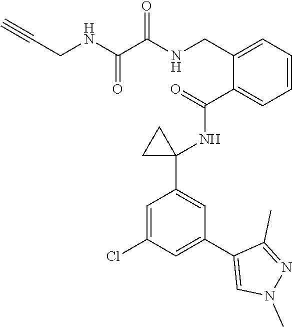 [SMILES: C#CCNC(=O)C(=O)NCc1ccccc1C(=O)NC1(c2cc(Cl)cc(-c3cn(C)nc3C)c2)CC1] | 504.2 |
| 486 | 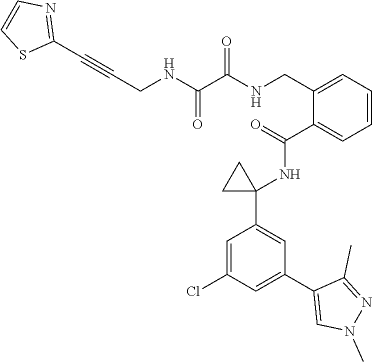 [SMILES: Cc1nn(C)cc1-c1cc(Cl)cc(C2(NC(=O)c3ccccc3CNC(=O)C(=O)NCC#Cc3nccs3)CC2)c1] | 588.9 |
| 487 | 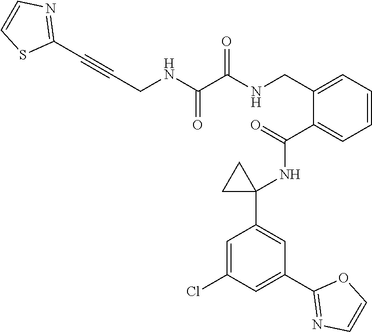 [SMILES: O=C(NCC#Cc1nccs1)C(=O)NCc1ccccc1C(=O)NC1(c2cc(Cl)cc(-c3ncco3)c2)CC1] | 561.9 |
| 488 | 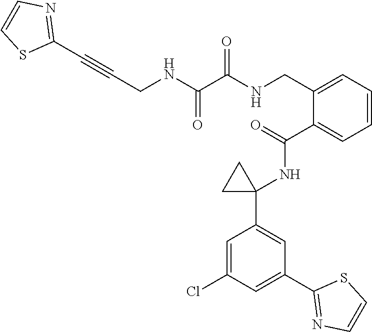 [SMILES: O=C(NCC#Cc1nccs1)C(=O)NCc1ccccc1C(=O)NC1(c2cc(Cl)cc(-c3nccs3)c2)CC1] | 577.8 |
| 489 | 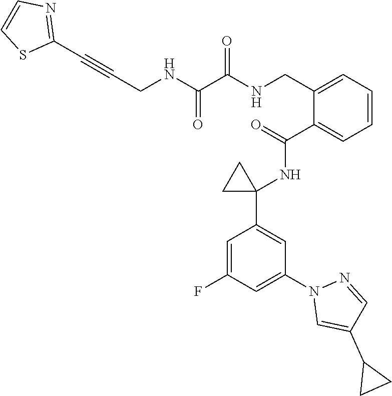 [SMILES: O=C(NCC#Cc1nccs1)C(=O)NCc1ccccc1C(=O)NC1(c2cc(F)cc(-n3cc(C4CC4)cn3)c2)CC1] | — |
| 490 | 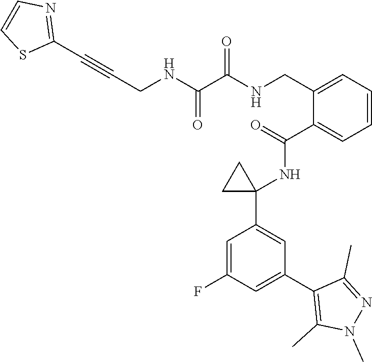 [SMILES: Cc1nn(C)c(C)c1-c1cc(F)cc(C2(NC(=O)c3ccccc3CNC(=O)C(=O)NCC#Cc3nccs3)CC2)c1] | 585.1 |
| 491 | 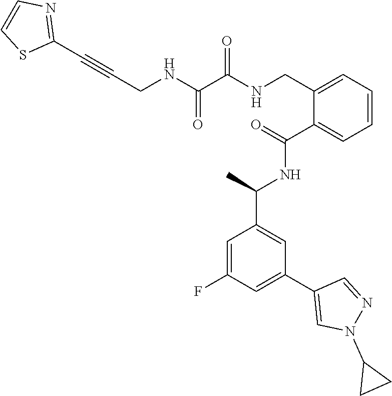 [SMILES: C[C@@H](NC(=O)c1ccccc1CNC(=O)C(=O)NCC#Cc1nccs1)c1cc(F)cc(-c2cnn(C3CC3)c2)c1] | 571.1 |
| 492 | 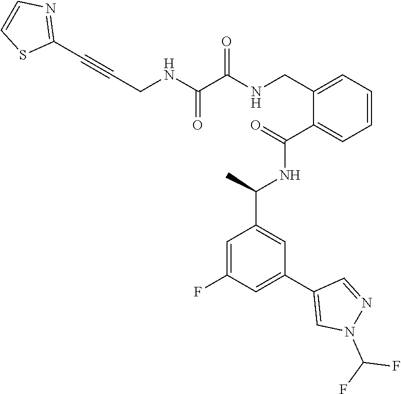 [SMILES: C[C@@H](NC(=O)c1ccccc1CNC(=O)C(=O)NCC#Cc1nccs1)c1cc(F)cc(-c2cnn(C(F)F)c2)c1] | 581.0 |
| 493 | 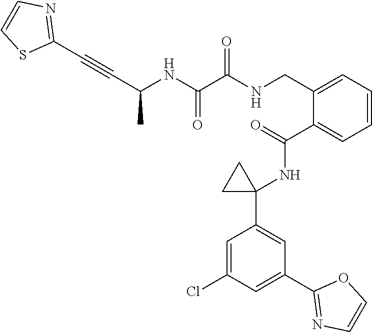 [SMILES: C[C@@H](C#Cc1nccs1)NC(=O)C(=O)NCc1ccccc1C(=O)NC1(c2cc(Cl)cc(-c3ncco3)c2)CC1] | 575.9 |
| 494 | 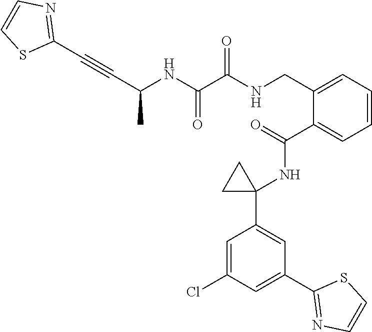 [SMILES: C[C@@H](C#Cc1nccs1)NC(=O)C(=O)NCc1ccccc1C(=O)NC1(c2cc(Cl)cc(-c3nccs3)c2)CC1] | 592.0 |
| 495 | 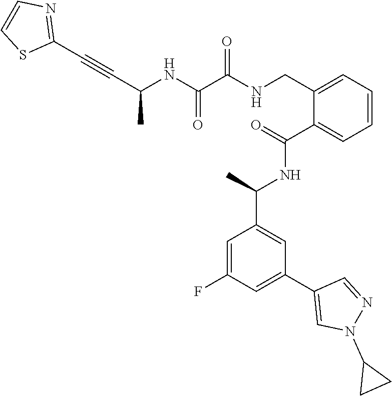 [SMILES: C[C@@H](C#Cc1nccs1)NC(=O)C(=O)NCc1ccccc1C(=O)N[C@H](C)c1cc(F)cc(-c2cnn(C3CC3)c2)c1] | 585.3 |
| 496 | 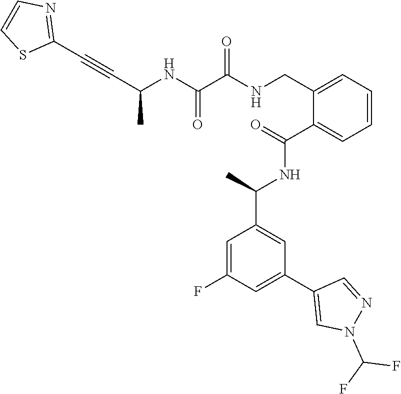 [SMILES: C[C@@H](C#Cc1nccs1)NC(=O)C(=O)NCc1ccccc1C(=O)N[C@H](C)c1cc(F)cc(-c2cnn(C(F)F)c2)c1] | 595.2 |
| 497 | 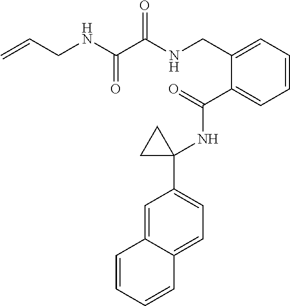 [SMILES: C=CCNC(=O)C(=O)NCc1ccccc1C(=O)NC1(c2ccc3ccccc3c2)CC1] | 428.2 |
| 498 | 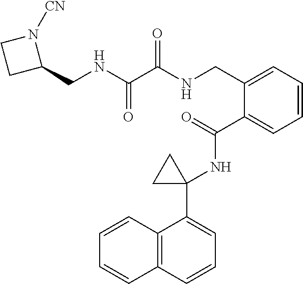 [SMILES: N#CN1CC[C@@H]1CNC(=O)C(=O)NCc1ccccc1C(=O)NC1(c2cccc3ccccc23)CC1] | 482.1 |
| 499 | 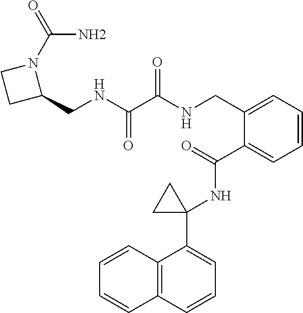 [SMILES: NC(=O)N1CC[C@@H]1CNC(=O)C(=O)NCc1ccccc1C(=O)NC1(c2cccc3ccccc23)CC1] | 500.2 |
| 500 | 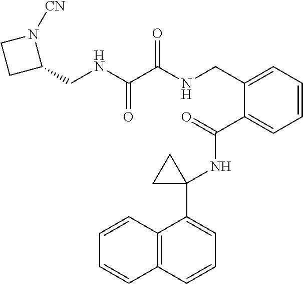 [SMILES: N#CN1CC[C@H]1CNC(=O)C(=O)NCc1ccccc1C(=O)NC1(c2cccc3ccccc23)CC1] | 482.2 |
| 501 | 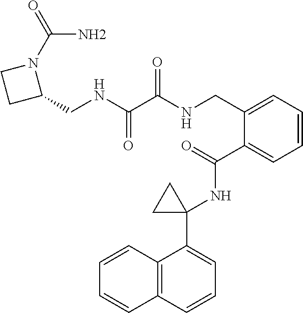 [SMILES: NC(=O)N1CC[C@H]1CNC(=O)C(=O)NCc1ccccc1C(=O)NC1(c2cccc3ccccc23)CC1] | 500.2 |
| 502 | 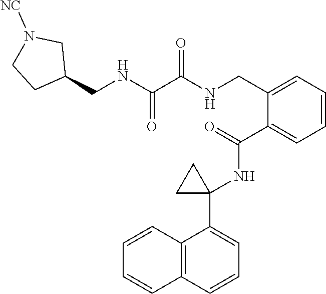 [SMILES: N#CN1CC[C@H](CNC(=O)C(=O)NCc2ccccc2C(=O)NC2(c3cccc4ccccc34)CC2)C1] | 496.2 |
| 503 | 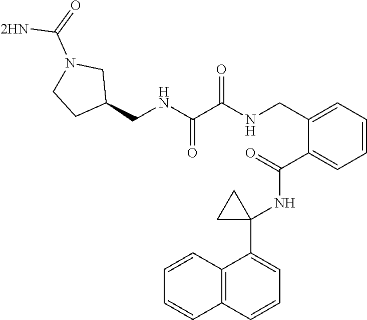 | 514.3 |
| 504 | 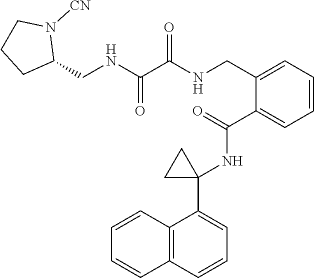 [SMILES: N#CN1CCC[C@H]1CNC(=O)C(=O)NCc1ccccc1C(=O)NC1(c2cccc3ccccc23)CC1] | 496.1 |
| 505 | 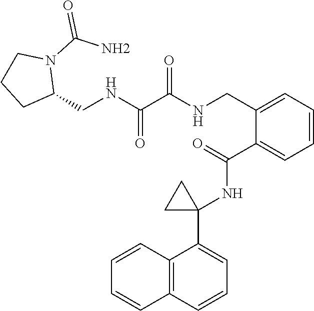 [SMILES: NC(=O)N1CCC[C@H]1CNC(=O)C(=O)NCc1ccccc1C(=O)NC1(c2cccc3ccccc23)CC1] | 514.2 |
| 506 | 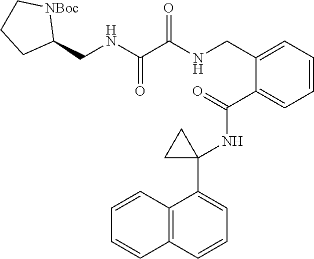 [SMILES: CC(C)(C)OC(=O)N1CCC[C@@H]1CNC(=O)C(=O)NCc1ccccc1C(=O)NC1(c2cccc3ccccc23)CC1] | 571.2 |
| 507 | 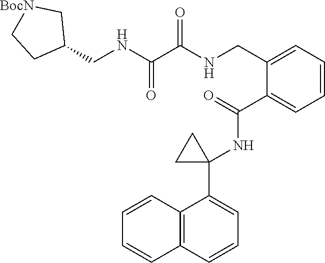 [SMILES: CC(C)(C)OC(=O)N1CC[C@@H](CNC(=O)C(=O)NCc2ccccc2C(=O)NC2(c3cccc4ccccc34)CC2)C1] | 571.2 |
| 508 | 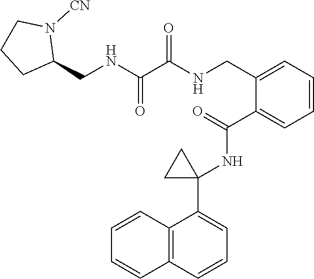 [SMILES: N#CN1CCC[C@@H]1CNC(=O)C(=O)NCc1ccccc1C(=O)NC1(c2cccc3ccccc23)CC1] | 496.3 |
| 509 | 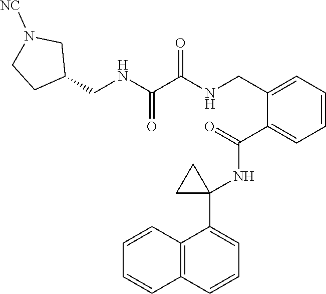 [SMILES: N#CN1CC[C@@H](CNC(=O)C(=O)NCc2ccccc2C(=O)NC2(c3cccc4ccccc34)CC2)C1] | 496.3 |
| 510 | 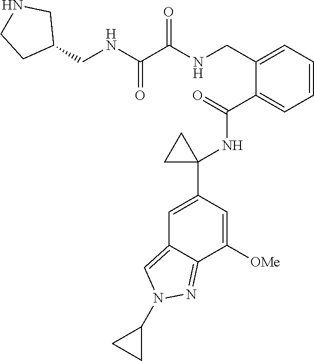 [SMILES: COc1cc(C2(NC(=O)c3ccccc3CNC(=O)C(=O)NC[C@@H]3CCNC3)CC2)cc2cn(C3CC3)nc12] | 531.1 |
| 511 | 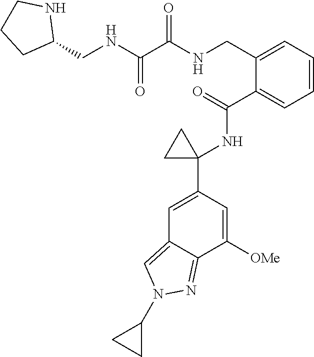 [SMILES: COc1cc(C2(NC(=O)c3ccccc3CNC(=O)C(=O)NC[C@@H]3CCCN3)CC2)cc2cn(C3CC3)nc12] | 531.2 |
| 512 | 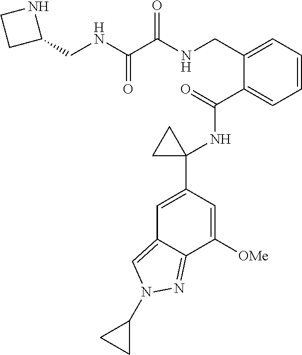 [SMILES: COc1cc(C2(NC(=O)c3ccccc3CNC(=O)C(=O)NC[C@@H]3CCN3)CC2)cc2cn(C3CC3)nc12] | 517.1 |
| 513 | 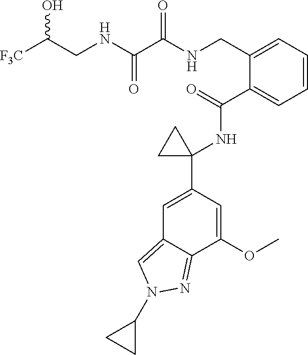 [SMILES: COc1cc(C2(NC(=O)c3ccccc3CNC(=O)C(=O)NCC(O)C(F)(F)F)CC2)cc2cn(C3CC3)nc12] | 560.2 |
| 514 | 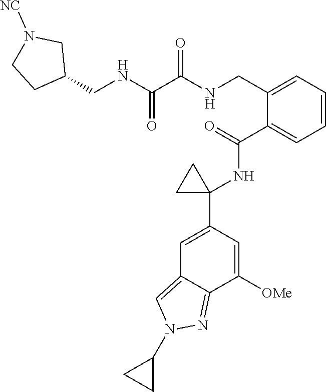 [SMILES: COc1cc(C2(NC(=O)c3ccccc3CNC(=O)C(=O)NC[C@@H]3CCN(C#N)C3)CC2)cc2cn(C3CC3)nc12] | — |
| 515 | 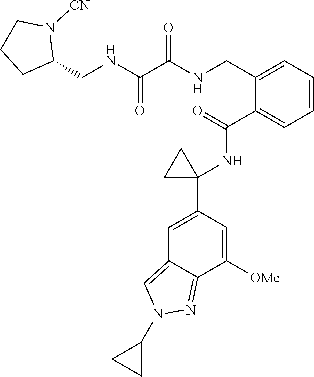 [SMILES: COc1cc(C2(NC(=O)c3ccccc3CNC(=O)C(=O)NC[C@@H]3CCCN3C#N)CC2)cc2cn(C3CC3)nc12] | — |
| 516 | 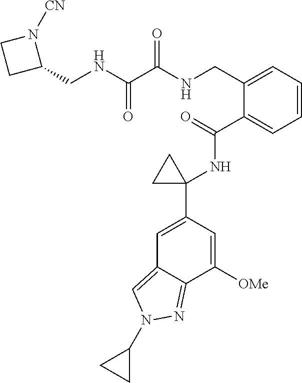 [SMILES: COc1cc(C2(NC(=O)c3ccccc3CNC(=O)C(=O)NC[C@@H]3CCN3C#N)CC2)cc2cn(C3CC3)nc12] | 542.3 |
| 517 | 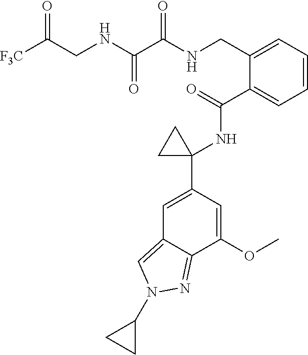 [SMILES: COc1cc(C2(NC(=O)c3ccccc3CNC(=O)C(=O)NCC(=O)C(F)(F)F)CC2)cc2cn(C3CC3)nc12] | — |
| 519 | 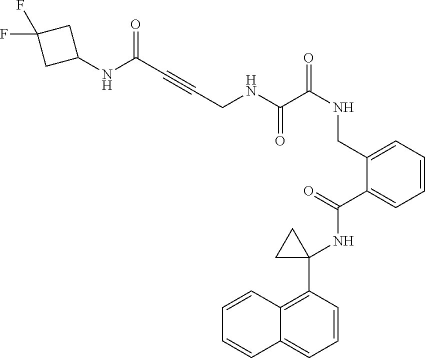 [SMILES: O=C(C#CCNC(=O)C(=O)NCc1ccccc1C(=O)NC1(c2cccc3ccccc23)CC1)NC1CC(F)(F)C1] | 557.19 [M − H]⁻ |
| 520 | 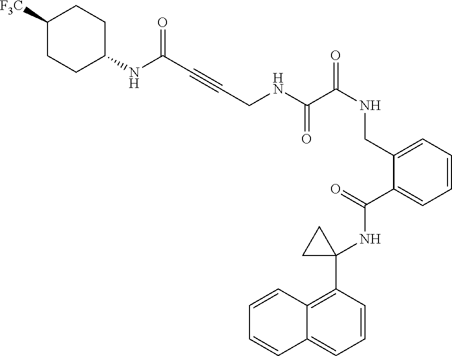 [SMILES: O=C(C#CCNC(=O)C(=O)NCc1ccccc1C(=O)NC1(c2cccc3ccccc23)CC1)N[C@H]1CC[C@H](C(F)(F)F)CC1] | 619.20 |
| 521 | 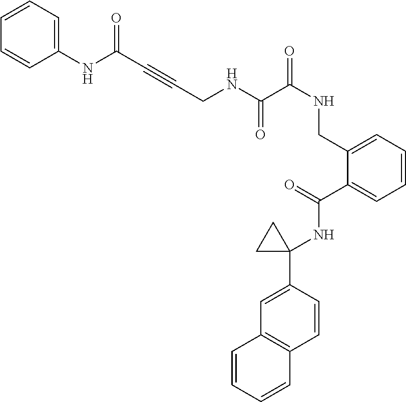 [SMILES: O=C(C#CCNC(=O)C(=O)NCc1ccccc1C(=O)NC1(c2ccc3ccccc3c2)CC1)Nc1ccccc1] | 545.19 |
| 522 | 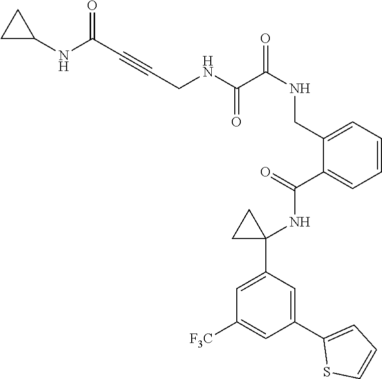 [SMILES: O=C(C#CCNC(=O)C(=O)NCc1ccccc1C(=O)NC1(c2cc(-c3cccs3)cc(C(F)(F)F)c2)CC1)NC1CC1] | 609.21 |
| 523 | 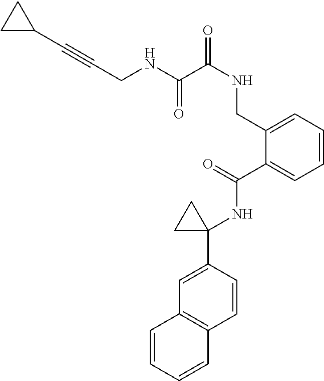 [SMILES: O=C(NCC#CC1CC1)C(=O)NCc1ccccc1C(=O)NC1(c2ccc3ccccc3c2)CC1] | 466.20 |
| 524 | 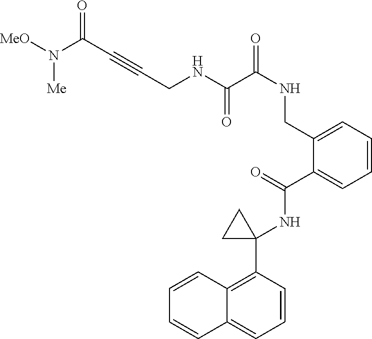 [SMILES: CON(C)C(=O)C#CCNC(=O)C(=O)NCc1ccccc1C(=O)NC1(c2cccc3ccccc23)CC1] | 513.25 |
| 525 | 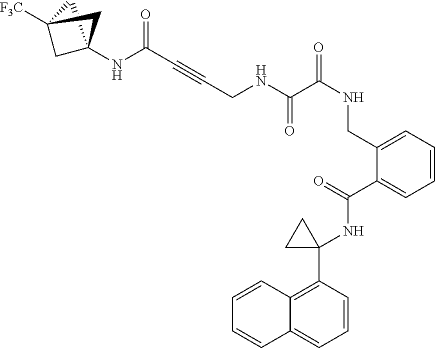 [SMILES: O=C(C#CCNC(=O)C(=O)NCc1ccccc1C(=O)NC1(c2cccc3ccccc23)CC1)N[C@]12C[C@](C(F)(F)F)(C1)C2] | 603.33 |
| 526 | 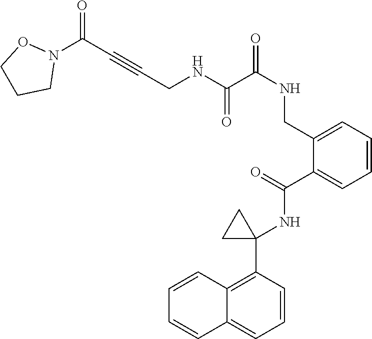 [SMILES: O=C(NCC#CC(=O)N1CCCO1)C(=O)NCc1ccccc1C(=O)NC1(c2cccc3ccccc23)CC1] | 525.31 |
| 527 | 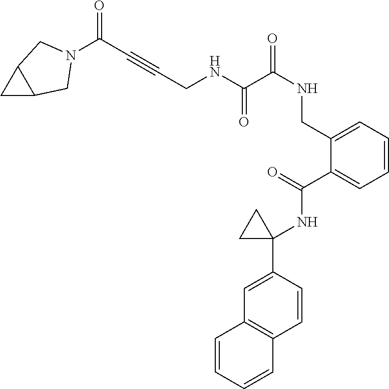 [SMILES: O=C(NCC#CC(=O)N1CC2CC2C1)C(=O)NCc1ccccc1C(=O)NC1(c2ccc3ccccc3c2)CC1] | 535.33 |
| 528 | 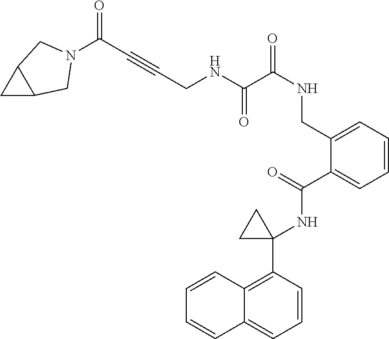 [SMILES: O=C(NCC#CC(=O)N1CC2CC2C1)C(=O)NCc1ccccc1C(=O)NC1(c2cccc3ccccc23)CC1] | 533.28 |
| 529 | 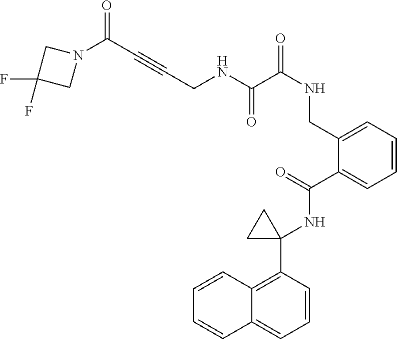 [SMILES: O=C(NCC#CC(=O)N1CC(F)(F)C1)C(=O)NCc1ccccc1C(=O)NC1(c2cccc3ccccc23)CC1] | 545.29 |
| 530 | 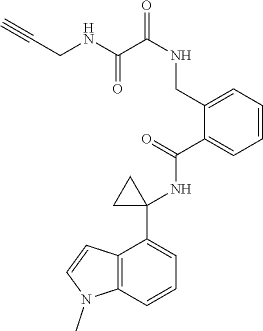 [SMILES: C#CCNC(=O)C(=O)NCc1ccccc1C(=O)NC1(c2cccc3c2ccn3C)CC1] | 429.15 |
| 531 | 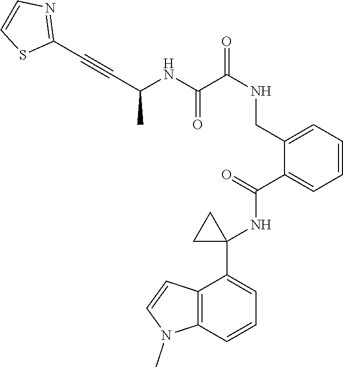 [SMILES: C[C@@H](C#Cc1nccs1)NC(=O)C(=O)NCc1ccccc1C(=O)NC1(c2cccc3c2ccn3C)CC1] | 526.20 |
| 532 | 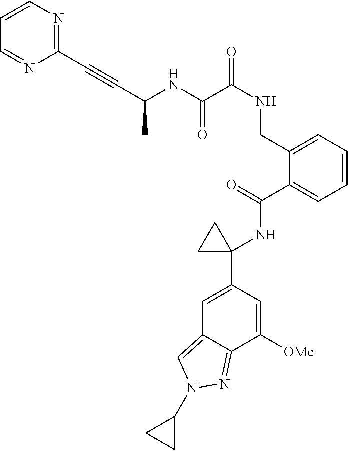 [SMILES: COc1cc(C2(NC(=O)c3ccccc3CNC(=O)C(=O)N[C@@H](C)C#Cc3ncccn3)CC2)cc2cn(C3CC3)nc12] | 578.30 |
| 533 | 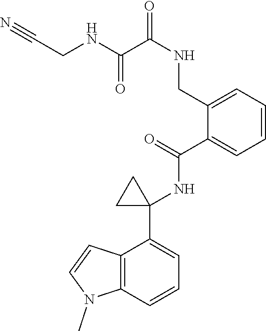 [SMILES: Cn1ccc2c(C3(NC(=O)c4ccccc4CNC(=O)C(=O)NCC#N)CC3)cccc21] | 430.22 |
| 534 | 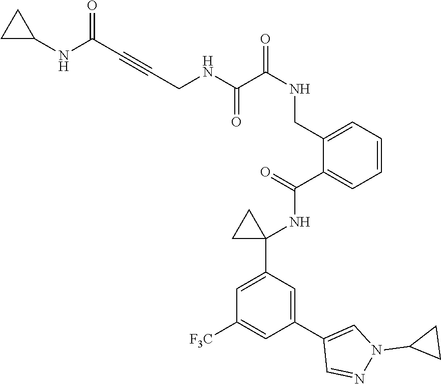 [SMILES: O=C(C#CCNC(=O)C(=O)NCc1ccccc1C(=O)NC1(c2cc(-c3cnn(C4CC4)c3)cc(C(F)(F)F)c2)CC1)NC1CC1] | 633.26 |
| 535 | 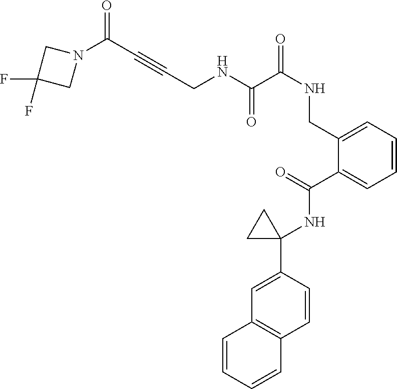 [SMILES: O=C(NCC#CC(=O)N1CC(F)(F)C1)C(=O)NCc1ccccc1C(=O)NC1(c2ccc3ccccc3c2)CC1] | 545.19 |
| 536 | 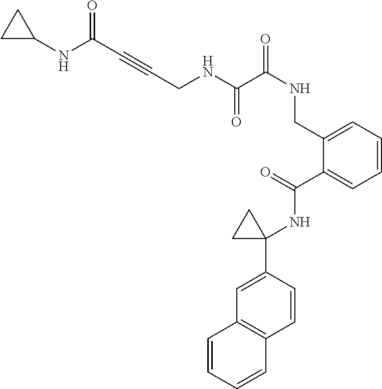 [SMILES: O=C(C#CCNC(=O)C(=O)NCc1ccccc1C(=O)NC1(c2ccc3ccccc3c2)CC1)NC1CC1] | 509.22 |
| 537 | 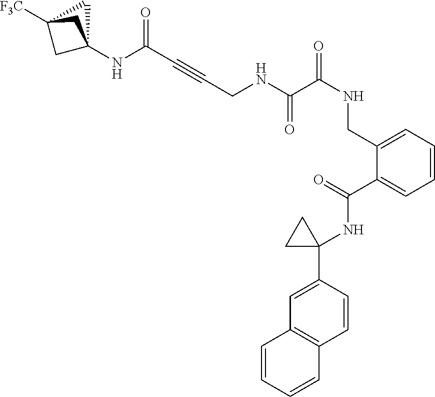 [SMILES: O=C(C#CCNC(=O)C(=O)NCc1ccccc1C(=O)NC1(c2ccc3ccccc3c2)CC1)N[C@]12C[C@](C(F)(F)F)(C1)C2] | 603.25 |
| 539 | 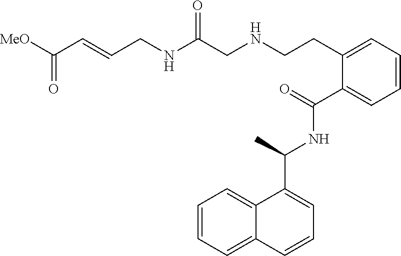 [SMILES: COC(=O)/C=C/CNC(=O)CNCCc1ccccc1C(=O)N[C@H](C)c1cccc2ccccc12] | 488.20 |
| 540 | 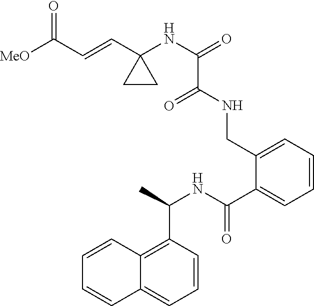 [SMILES: COC(=O)/C=C/C1(NC(=O)C(=O)NCc2ccccc2C(=O)N[C@H](C)c2cccc3ccccc23)CC1] | 500.20 |
| 541 | 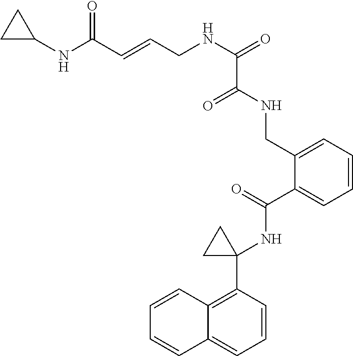 [SMILES: O=C(/C=C/CNC(=O)C(=O)NCc1ccccc1C(=O)NC1(c2cccc3ccccc23)CC1)NC1CC1] | 511.20 |
| 542 | 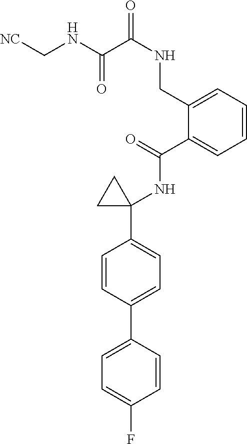 [SMILES: N#CCNC(=O)C(=O)NCc1ccccc1C(=O)NC1(c2ccc(-c3ccc(F)cc3)cc2)CC1] | 493.10 [M + Na]⁺ |
| 543 | 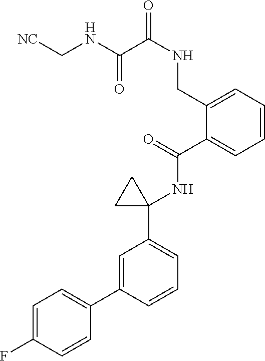 [SMILES: N#CCNC(=O)C(=O)NCc1ccccc1C(=O)NC1(c2cccc(-c3ccc(F)cc3)c2)CC1] | 471.25 |
| 544 | 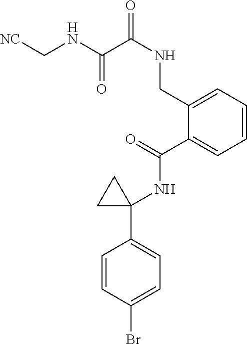 [SMILES: N#CCNC(=O)C(=O)NCc1ccccc1C(=O)NC1(c2ccc(Br)cc2)CC1] | 454.95 |
| 545 | 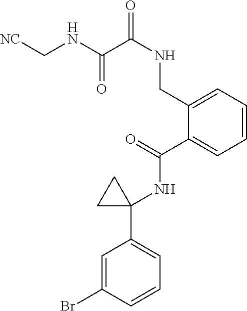 [SMILES: N#CCNC(=O)C(=O)NCc1ccccc1C(=O)NC1(c2cccc(Br)c2)CC1] | 455.15, |
| 546 | 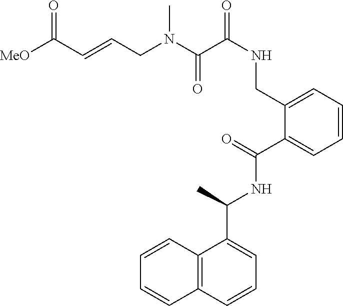 [SMILES: COC(=O)/C=C/CN(C)C(=O)C(=O)NCc1ccccc1C(=O)N[C@H](C)c1cccc2ccccc12] | 488.30 |
| 547 | 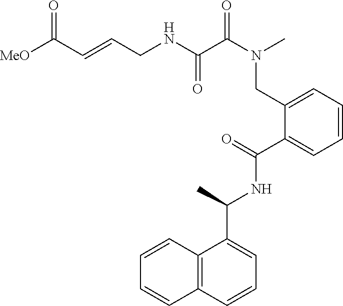 [SMILES: COC(=O)/C=C/CNC(=O)C(=O)N(C)Cc1ccccc1C(=O)N[C@H](C)c1cccc2ccccc12] | 488.30 |
| 548 | 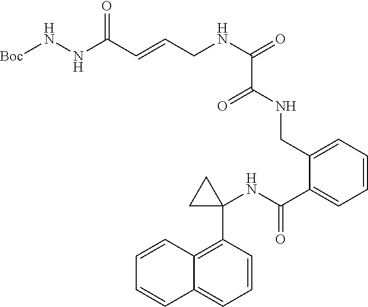 [SMILES: CC(C)(C)OC(=O)NNC(=O)/C=C/CNC(=O)C(=O)NCc1ccccc1C(=O)NC1(c2cccc3ccccc23)CC1] | 608.25 [M + Na]⁺ |
| 549 | 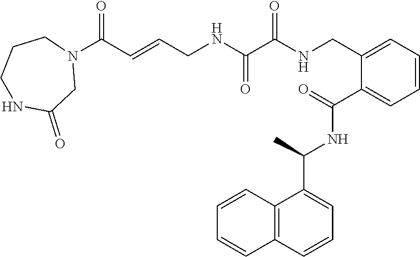 [SMILES: C[C@@H](NC(=O)c1ccccc1CNC(=O)C(=O)NC/C=C/C(=O)N1CCCNC(=O)C1)c1cccc2ccccc12] | 556.10 |
| 550 | 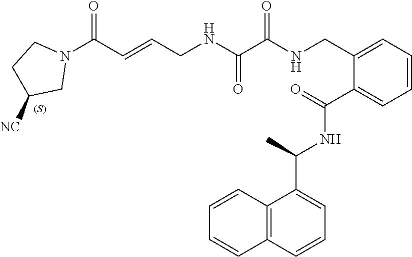 | 538.25 |
| 551 | 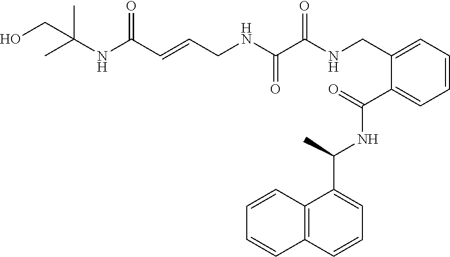 [SMILES: C[C@@H](NC(=O)c1ccccc1CNC(=O)C(=O)NC/C=C/C(=O)NC(C)(C)CO)c1cccc2ccccc12] | 531.10 |
| 552 | 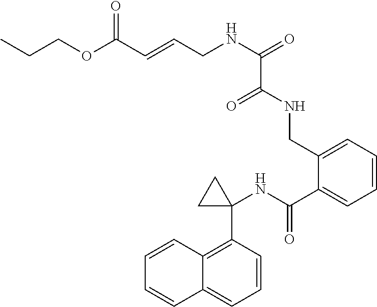 [SMILES: CCCOC(=O)/C=C/CNC(=O)C(=O)NCc1ccccc1C(=O)NC1(c2cccc3ccccc23)CC1] | 514.20 |
| 553 | 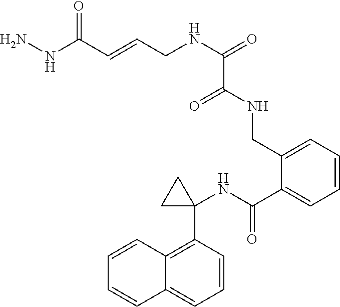 [SMILES: NNC(=O)/C=C/CNC(=O)C(=O)NCc1ccccc1C(=O)NC1(c2cccc3ccccc23)CC1] | 486.20 |
| 554 | 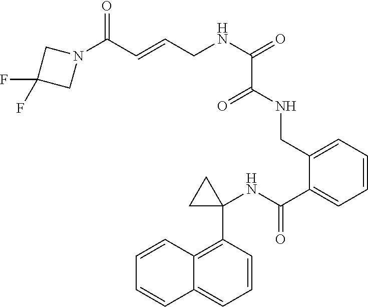 [SMILES: O=C(NC/C=C/C(=O)N1CC(F)(F)C1)C(=O)NCc1ccccc1C(=O)NC1(c2cccc3ccccc23)CC1] | 547.25 |
| 555 | 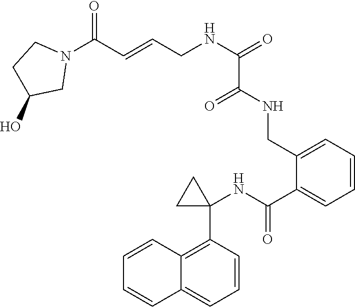 [SMILES: O=C(NC/C=C/C(=O)N1CC[C@H](O)C1)C(=O)NCc1ccccc1C(=O)NC1(c2cccc3ccccc23)CC1] | 541.25 |
| 556 | 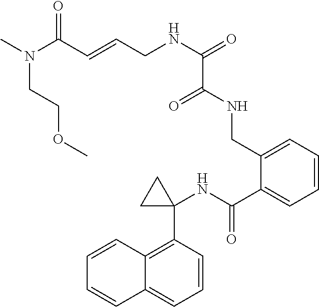 [SMILES: COCCN(C)C(=O)/C=C/CNC(=O)C(=O)NCc1ccccc1C(=O)NC1(c2cccc3ccccc23)CC1] | 543.30 |
| 557 | 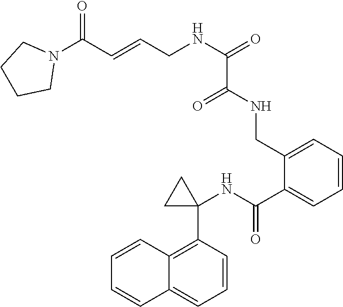 [SMILES: O=C(NC/C=C/C(=O)N1CCCC1)C(=O)NCc1ccccc1C(=O)NC1(c2cccc3ccccc23)CC1] | 525.30 |
| 558 | 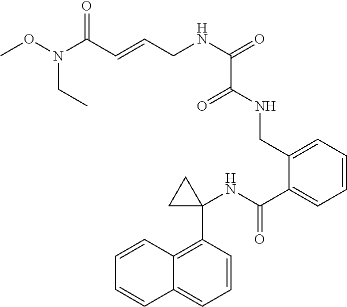 [SMILES: CCN(OC)C(=O)/C=C/CNC(=O)C(=O)NCc1ccccc1C(=O)NC1(c2cccc3ccccc23)CC1] | 529.25 |
| 559 | 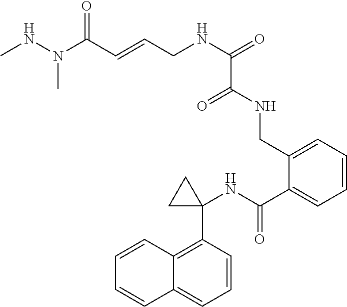 [SMILES: CNN(C)C(=O)/C=C/CNC(=O)C(=O)NCc1ccccc1C(=O)NC1(c2cccc3ccccc23)CC1] | 514.25 |
| 560 | 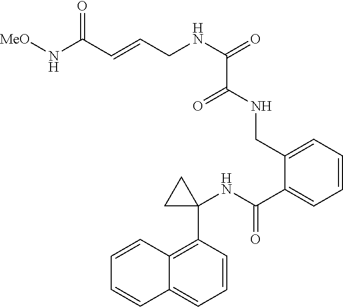 [SMILES: CONC(=O)/C=C/CNC(=O)C(=O)NCc1ccccc1C(=O)NC1(c2cccc3ccccc23)CC1] | 501.25 |
| 561 | 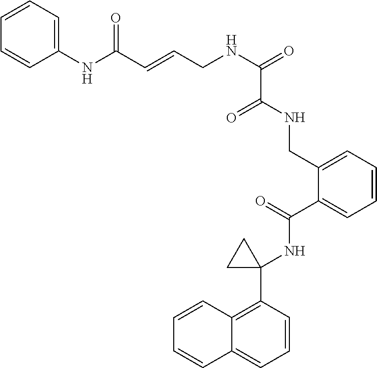 [SMILES: O=C(/C=C/CNC(=O)C(=O)NCc1ccccc1C(=O)NC1(c2cccc3ccccc23)CC1)Nc1ccccc1] | 547.25 |
| 562 | 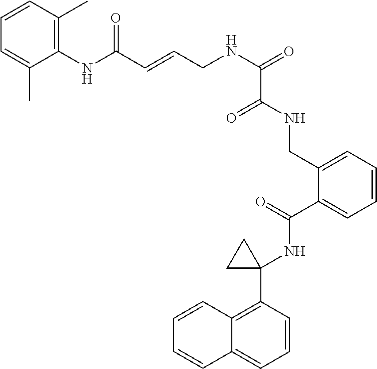 [SMILES: Cc1cccc(C)c1NC(=O)/C=C/CNC(=O)C(=O)NCc1ccccc1C(=O)NC1(c2cccc3ccccc23)CC1] | 575.25 |
| 563 | 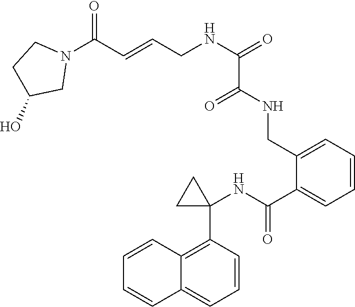 [SMILES: O=C(NC/C=C/C(=O)N1CC[C@@H](O)C1)C(=O)NCc1ccccc1C(=O)NC1(c2cccc3ccccc23)CC1] | 541.25 |
| 564 | 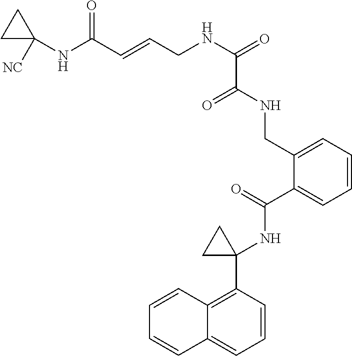 [SMILES: N#CC1(NC(=O)/C=C/CNC(=O)C(=O)NCc2ccccc2C(=O)NC2(c3cccc4ccccc34)CC2)CC1] | 536.25 |
| 565 | 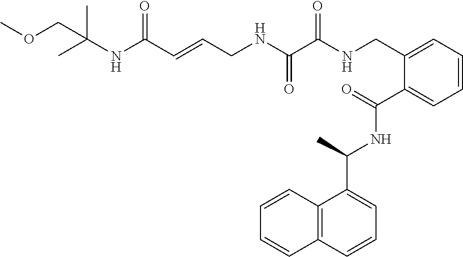 [SMILES: COCC(C)(C)NC(=O)/C=C/CNC(=O)C(=O)NCc1ccccc1C(=O)N[C@H](C)c1cccc2ccccc12] | 545.30 |
| 566 | 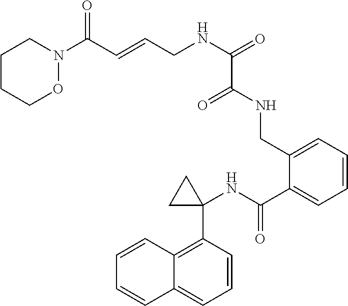 [SMILES: O=C(NC/C=C/C(=O)N1CCCCO1)C(=O)NCc1ccccc1C(=O)NC1(c2cccc3ccccc23)CC1] | 563.25 [M + Na]+ |
| 567 | 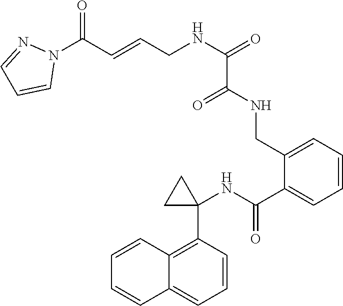 [SMILES: O=C(NC/C=C/C(=O)n1cccn1)C(=O)NCc1ccccc1C(=O)NC1(c2cccc3ccccc23)CC1] | 522.25 |
| 568 | 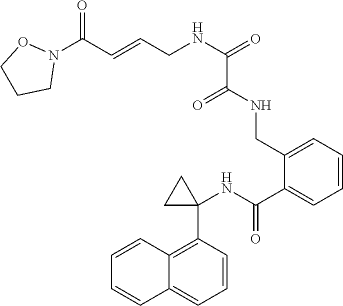 [SMILES: O=C(NC/C=C/C(=O)N1CCCO1)C(=O)NCc1ccccc1C(=O)NC1(c2cccc3ccccc23)CC1] | 527.30 |
| 569 | 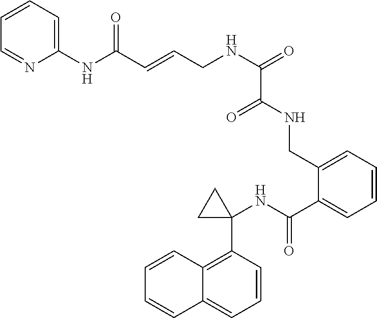 [SMILES: O=C(/C=C/CNC(=O)C(=O)NCc1ccccc1C(=O)NC1(c2cccc3ccccc23)CC1)Nc1ccccn1] | 548.30 |
| 571 | 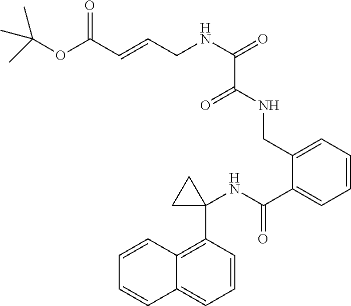 [SMILES: CC(C)(C)OC(=O)/C=C/CNC(=O)C(=O)NCc1ccccc1C(=O)NC1(c2cccc3ccccc23)CC1] | 528.30 |
| 572 | 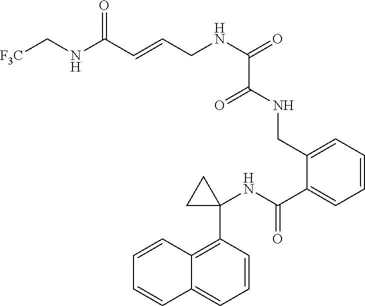 [SMILES: O=C(/C=C/CNC(=O)C(=O)NCc1ccccc1C(=O)NC1(c2cccc3ccccc23)CC1)NCC(F)(F)F] | 553.30 |
| 573 | 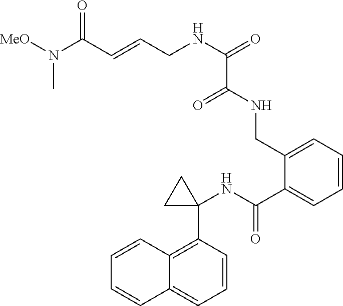 [SMILES: CON(C)C(=O)/C=C/CNC(=O)C(=O)NCc1ccccc1C(=O)NC1(c2cccc3ccccc23)CC1] | 515.25 |
| 574 | 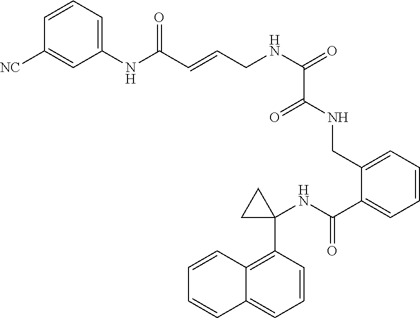 [SMILES: N#Cc1cccc(NC(=O)/C=C/CNC(=O)C(=O)NCc2ccccc2C(=O)NC2(c3cccc4ccccc34)CC2)c1] | 572.30 |
| 575 | 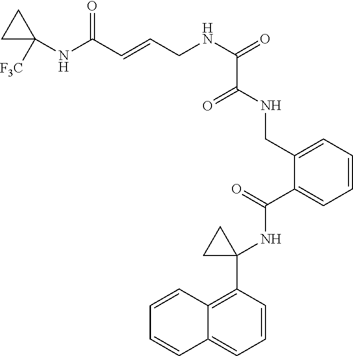 [SMILES: O=C(/C=C/CNC(=O)C(=O)NCc1ccccc1C(=O)NC1(c2cccc3ccccc23)CC1)NC1(C(F)(F)F)CC1] | 579.25 |
| 576 | 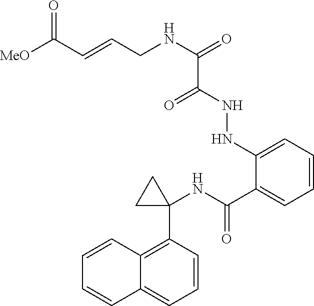 [SMILES: COC(=O)/C=C/CNC(=O)C(=O)NNc1ccccc1C(=O)NC1(c2cccc3ccccc23)CC1] | 487.20 |
| 577 | 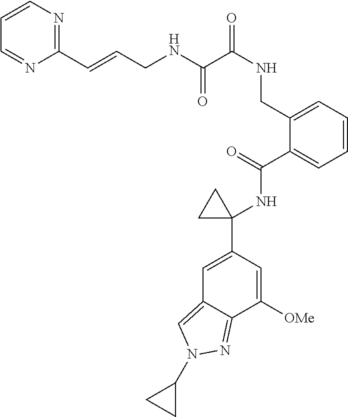 [SMILES: COc1cc(C2(NC(=O)c3ccccc3CNC(=O)C(=O)NC/C=C/c3ncccn3)CC2)cc2cn(C3CC3)nc12] | 566.30 |
| 578 | 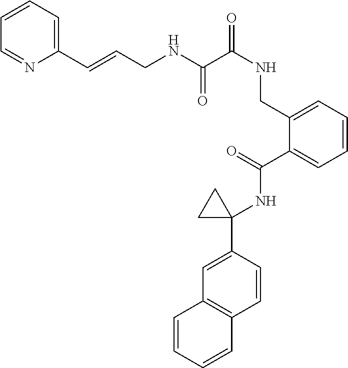 [SMILES: O=C(NC/C=C/c1ccccn1)C(=O)NCc1ccccc1C(=O)NC1(c2ccc3ccccc3c2)CC1] | 505.32 |
| 579 | 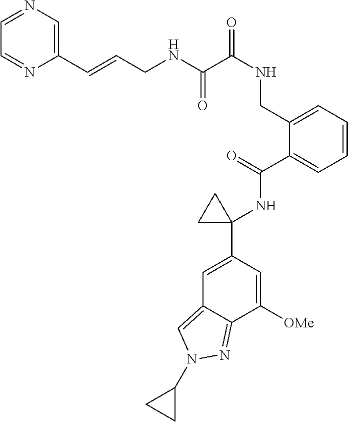 [SMILES: COc1cc(C2(NC(=O)c3ccccc3CNC(=O)C(=O)NC/C=C/c3cnccn3)CC2)cc2cn(C3CC3)nc12] | 566.35 |
| 580 | 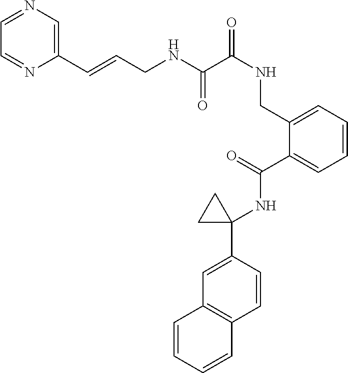 [SMILES: O=C(NC/C=C/c1cnccn1)C(=O)NCc1ccccc1C(=O)NC1(c2ccc3ccccc3c2)CC1] | 506.25 |
| 581 | 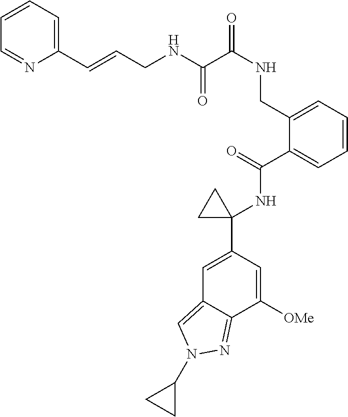 [SMILES: COc1cc(C2(NC(=O)c3ccccc3CNC(=O)C(=O)NC/C=C/c3ccccn3)CC2)cc2cn(C3CC3)nc12] | 565.34 |
| 582 | 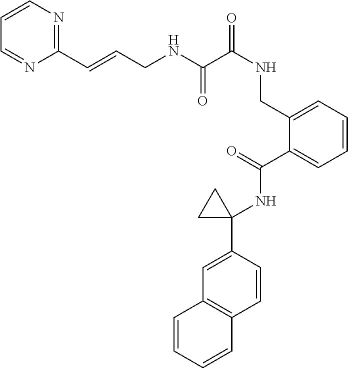 [SMILES: O=C(NC/C=C/c1ncccn1)C(=O)NCc1ccccc1C(=O)NC1(c2ccc3ccccc3c2)CC1] | 506.25 |
| 583 | 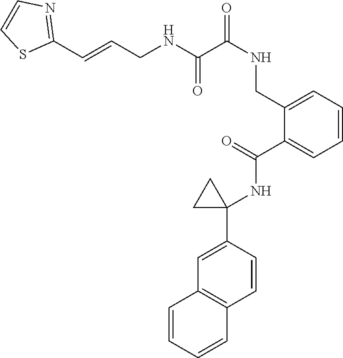 [SMILES: O=C(NC/C=C/c1nccs1)C(=O)NCc1ccccc1C(=O)NC1(c2ccc3ccccc3c2)CC1] | 511.23 |

TABLE 2
The following examples are prepared employing similar methods as described above.

| Example | Structure |
|---|---|
| 1a | 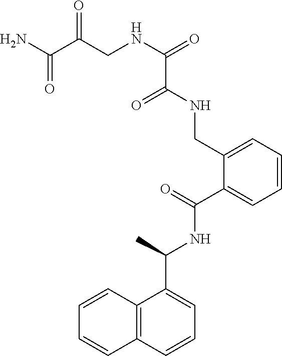 [SMILES: C[C@@H](NC(=O)c1ccccc1CNC(=O)C(=O)NCC(=O)C(N)=O)c1cccc2ccccc12] |
| 2a | 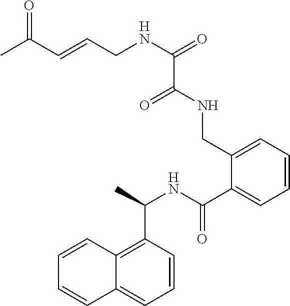 [SMILES: CC(=O)/C=C/CNC(=O)C(=O)NCc1ccccc1C(=O)N[C@H](C)c1cccc2ccccc12] |
| 3a | 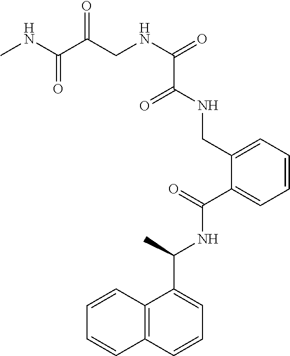 [SMILES: CNC(=O)C(=O)CNC(=O)C(=O)NCc1ccccc1C(=O)N[C@H](C)c1cccc2ccccc12] |
| 4a | 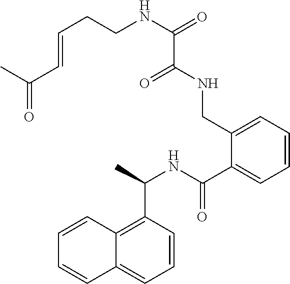 [SMILES: CC(=O)/C=C/CCNC(=O)C(=O)NCc1ccccc1C(=O)N[C@H](C)c1cccc2ccccc12] |
| 5a | 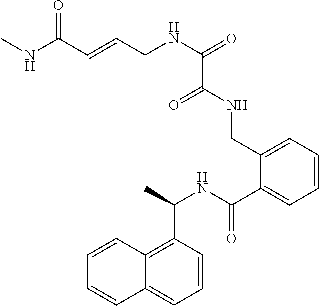 [SMILES: CNC(=O)/C=C/CNC(=O)C(=O)NCc1ccccc1C(=O)N[C@H](C)c1cccc2ccccc12] |
| 6a | 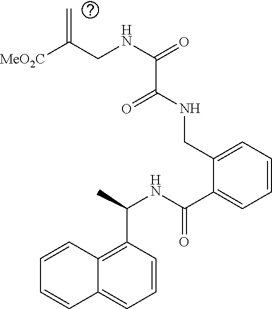 [SMILES: C=C(CNC(=O)C(=O)NCc1ccccc1C(=O)N[C@H](C)c1cccc2ccccc12)C(=O)OC] |
| 7a | 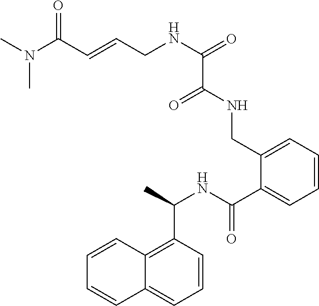 [SMILES: C[C@@H](NC(=O)c1ccccc1CNC(=O)C(=O)NC/C=C/C(=O)N(C)C)c1cccc2ccccc12] |
| 8a | 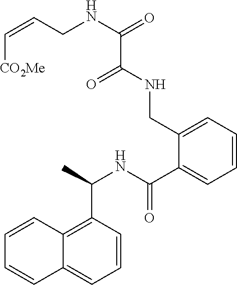 [SMILES: COC(=O)/C=C\CNC(=O)C(=O)NCc1ccccc1C(=O)N[C@H](C)c1cccc2ccccc12] |
| 9a | 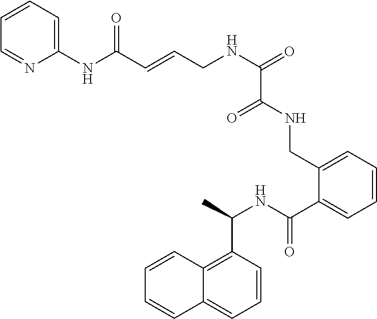 [SMILES: C[C@@H](NC(=O)c1ccccc1CNC(=O)C(=O)NC/C=C/C(=O)Nc1ccccn1)c1cccc2ccccc12] |
| 10a | 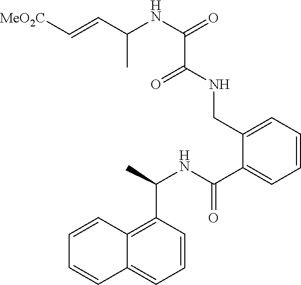 [SMILES: COC(=O)/C=C/C(C)NC(=O)C(=O)NCc1ccccc1C(=O)N[C@H](C)c1cccc2ccccc12] |
| 11a | 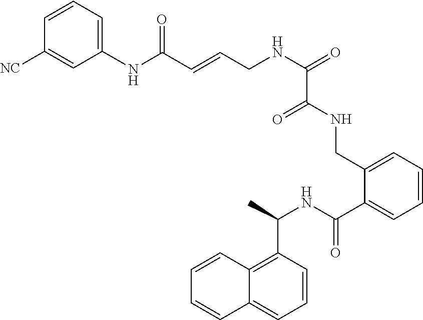 [SMILES: C[C@@H](NC(=O)c1ccccc1CNC(=O)C(=O)NC/C=C/C(=O)Nc1cccc(C#N)c1)c1cccc2ccccc12] |
| 12a | 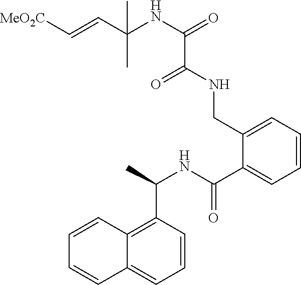 [SMILES: COC(=O)/C=C/C(C)(C)NC(=O)C(=O)NCc1ccccc1C(=O)N[C@H](C)c1cccc2ccccc12] |
| 13a | 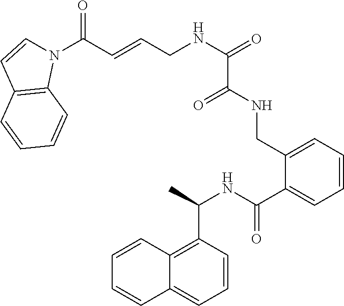 [SMILES: C[C@@H](NC(=O)c1ccccc1CNC(=O)C(=O)NC/C=C/C(=O)n1ccc2ccccc21)c1cccc2ccccc12] |
| 14a | 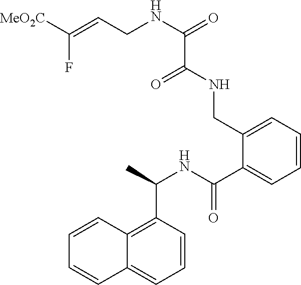 [SMILES: COC(=O)/C(F)=C/CNC(=O)C(=O)NCc1ccccc1C(=O)N[C@H](C)c1cccc2ccccc12] |
| 15a | 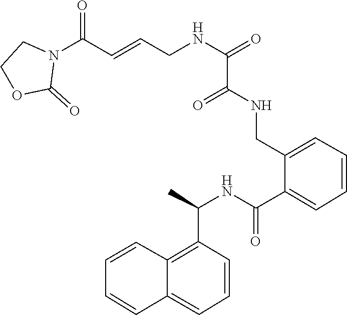 [SMILES: C[C@@H](NC(=O)c1ccccc1CNC(=O)C(=O)NC/C=C/C(=O)N1CCOC1=O)c1cccc2ccccc12] |
| 16a | 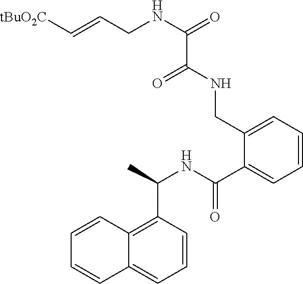 [SMILES: C[C@@H](NC(=O)c1ccccc1CNC(=O)C(=O)NC/C=C/C(=O)OC(C)(C)C)c1cccc2ccccc12] |
| 17a | 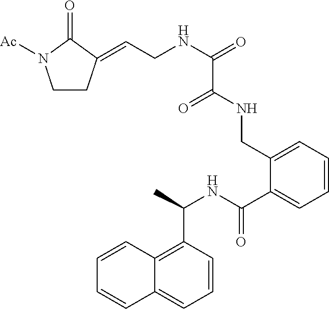 [SMILES: CC(=O)N1CC/C(=C\CNC(=O)C(=O)NCc2ccccc2C(=O)N[C@H](C)c2cccc3ccccc23)C1=O] |
| 18a | 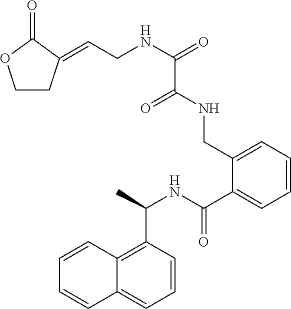 [SMILES: C[C@@H](NC(=O)c1ccccc1CNC(=O)C(=O)NC/C=C1\CCOC1=O)c1cccc2ccccc12] |
| 19a | 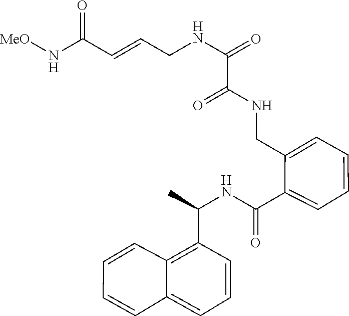 [SMILES: CONC(=O)/C=C/CNC(=O)C(=O)NCc1ccccc1C(=O)N[C@H](C)c1cccc2ccccc12] |
| 20a | 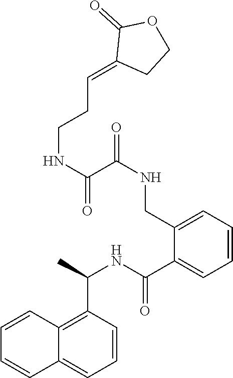 [SMILES: C[C@@H](NC(=O)c1ccccc1CNC(=O)C(=O)NCC/C=C1\CCOC1=O)c1cccc2ccccc12] |
| 21a | 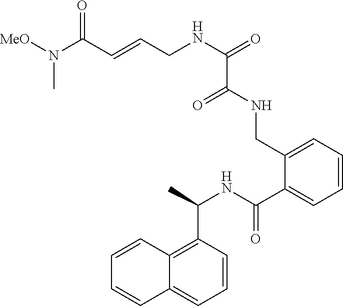 [SMILES: CON(C)C(=O)/C=C/CNC(=O)C(=O)NCc1ccccc1C(=O)N[C@H](C)c1cccc2ccccc12] |
| 22a | 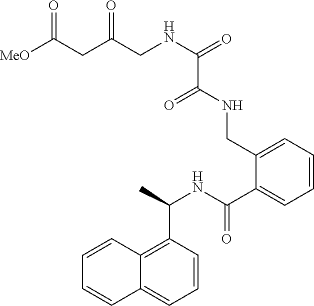 [SMILES: COC(=O)CC(=O)CNC(=O)C(=O)NCc1ccccc1C(=O)N[C@H](C)c1cccc2ccccc12] |
| 23a | 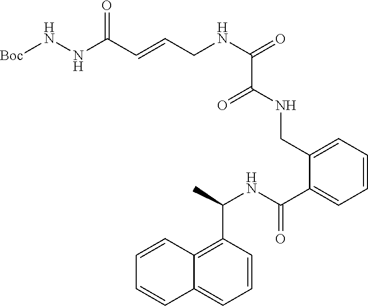 [SMILES: C[C@@H](NC(=O)c1ccccc1CNC(=O)C(=O)NC/C=C/C(=O)NNC(=O)OC(C)(C)C)c1cccc2ccccc12] |
| 24a | 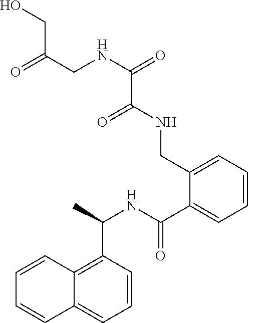 |
| 25a | 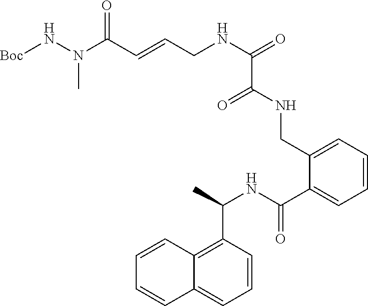 |
| 26a | 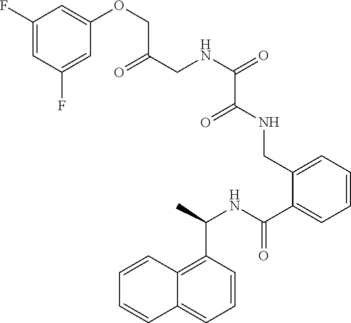 [SMILES: C[C@@H](NC(=O)c1ccccc1CNC(=O)C(=O)NCC(=O)COc1cc(F)cc(F)c1)c1cccc2ccccc12] |
| 27a | 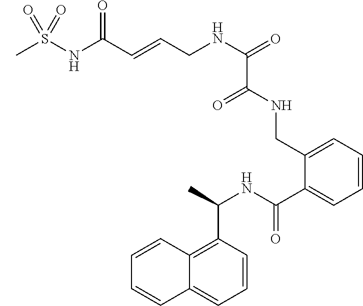 |
| 28a | 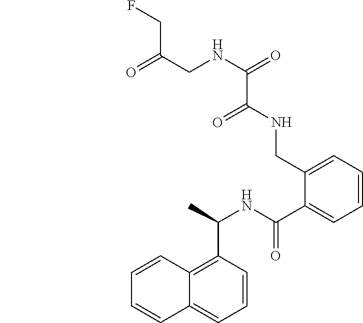 |
| 29a | 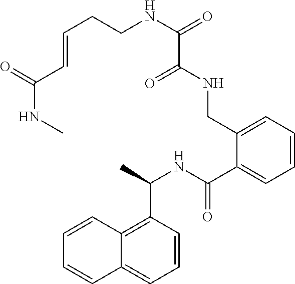 [SMILES: CNC(=O)/C=C/CCNC(=O)C(=O)NCc1ccccc1C(=O)N[C@H](C)c1cccc2ccccc12] |
| 30a | 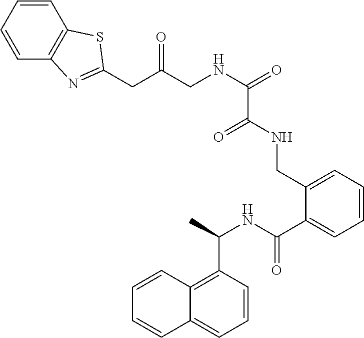 [SMILES: C[C@@H](NC(=O)c1ccccc1CNC(=O)C(=O)NCC(=O)Cc1nc2ccccc2s1)c1cccc2ccccc12] |
| 31a | 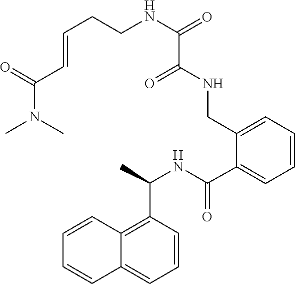 [SMILES: C[C@@H](NC(=O)c1ccccc1CNC(=O)C(=O)NCC/C=C/C(=O)N(C)C)c1cccc2ccccc12] |
| 32a | 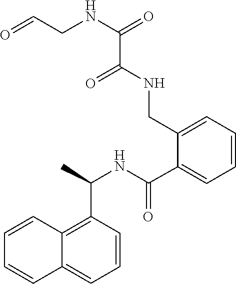 [SMILES: C[C@@H](NC(=O)c1ccccc1CNC(=O)C(=O)NCC=O)c1cccc2ccccc12] |
| 33a | 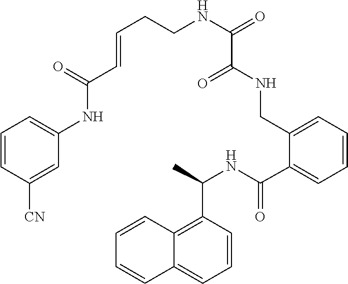 [SMILES: C[C@@H](NC(=O)c1ccccc1CNC(=O)C(=O)NCC/C=C/C(=O)Nc1cccc(C#N)c1)c1cccc2ccccc12] |
| 34a | 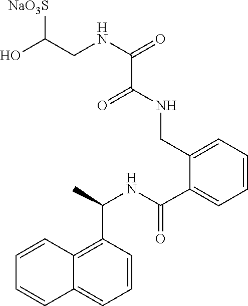 |
| 35a | 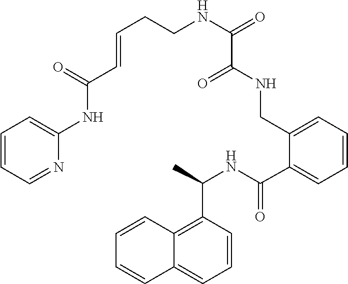 [SMILES: C[C@@H](NC(=O)c1ccccc1CNC(=O)C(=O)NCC/C=C/C(=O)Nc1ccccn1)c1cccc2ccccc12] |
| 36a | 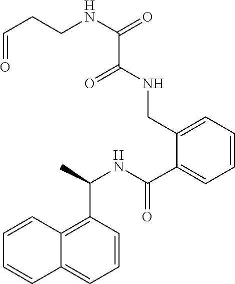 [SMILES: C[C@@H](NC(=O)c1ccccc1CNC(=O)C(=O)NCCC=O)c1cccc2ccccc12] |
| 37a | 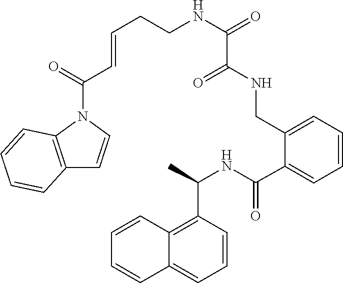 [SMILES: C[C@@H](NC(=O)c1ccccc1CNC(=O)C(=O)NCC/C=C/C(=O)n1ccc2ccccc21)c1cccc2ccccc12] |
| 38a | 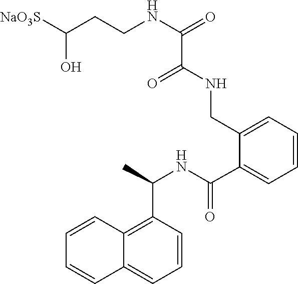 |
| 39a | 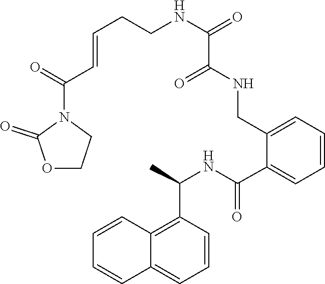 [SMILES: C[C@@H](NC(=O)c1ccccc1CNC(=O)C(=O)NCC/C=C/C(=O)N1CCOC1=O)c1cccc2ccccc12] |
| 40a | 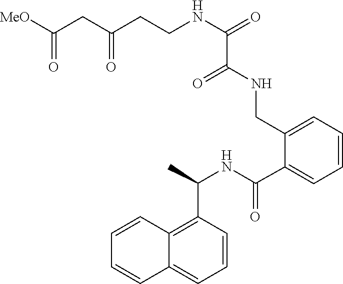 [SMILES: COC(=O)CC(=O)CCNC(=O)C(=O)NCc1ccccc1C(=O)N[C@H](C)c1cccc2ccccc12] |
| 41a | 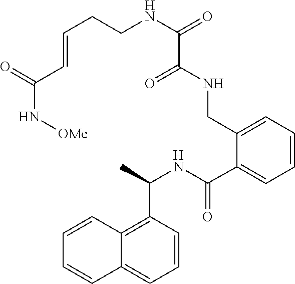 [SMILES: CONC(=O)/C=C/CCNC(=O)C(=O)NCc1ccccc1C(=O)N[C@H](C)c1cccc2ccccc12] |
| 42a | 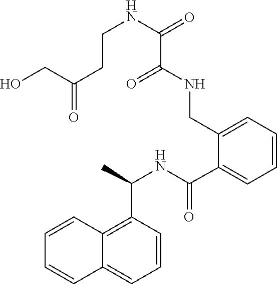 [SMILES: C[C@@H](NC(=O)c1ccccc1CNC(=O)C(=O)NCCC(=O)CO)c1cccc2ccccc12] |
| 43a | 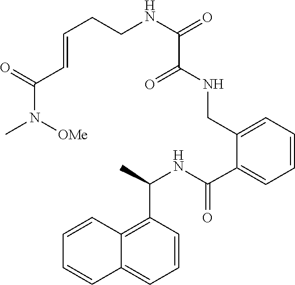 [SMILES: CON(C)C(=O)/C=C/CCNC(=O)C(=O)NCc1ccccc1C(=O)N[C@H](C)c1cccc2ccccc12] |
| 44a | 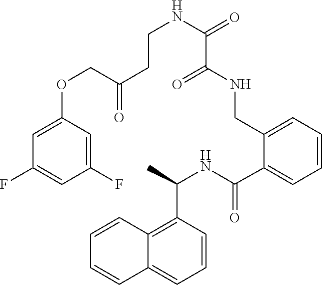 [SMILES: C[C@@H](NC(=O)c1ccccc1CNC(=O)C(=O)NCCC(=O)COc1cc(F)cc(F)c1)c1cccc2ccccc12] |
| 45a | 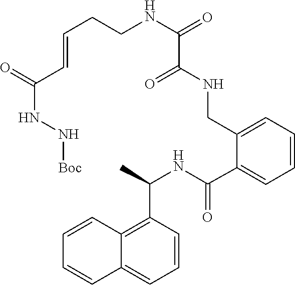 [SMILES: C[C@@H](NC(=O)c1ccccc1CNC(=O)C(=O)NCC/C=C/C(=O)NNC(=O)OC(C)(C)C)c1cccc2ccccc12] |
| 46a | 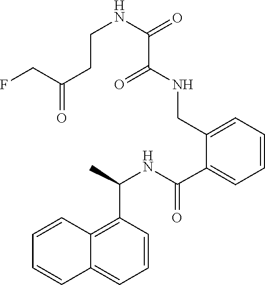 [SMILES: C[C@@H](NC(=O)c1ccccc1CNC(=O)C(=O)NCCC(=O)CF)c1cccc2ccccc12] |
| 47a | 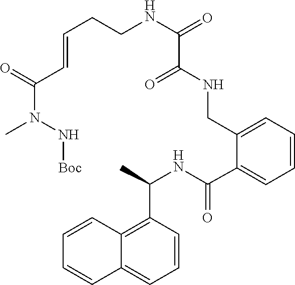 [SMILES: C[C@@H](NC(=O)c1ccccc1CNC(=O)C(=O)NCC/C=C/C(=O)N(C)NC(=O)OC(C)(C)C)c1cccc2ccccc12] |
| 48a | 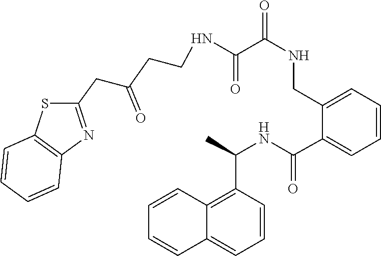 [SMILES: C[C@@H](NC(=O)c1ccccc1CNC(=O)C(=O)NCCC(=O)Cc1nc2ccccc2s1)c1cccc2ccccc12] |
| 49a | 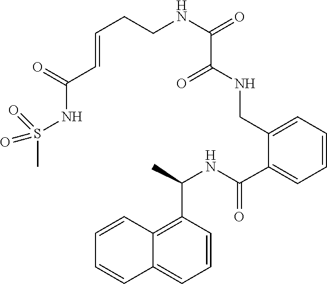 [SMILES: C[C@@H](NC(=O)c1ccccc1CNC(=O)C(=O)NCC/C=C/C(=O)NS(C)(=O)=O)c1cccc2ccccc12] |
| 50a | 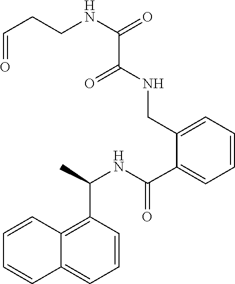 [SMILES: C[C@@H](NC(=O)c1ccccc1CNC(=O)C(=O)NCCC=O)c1cccc2ccccc12] |
| 51a | 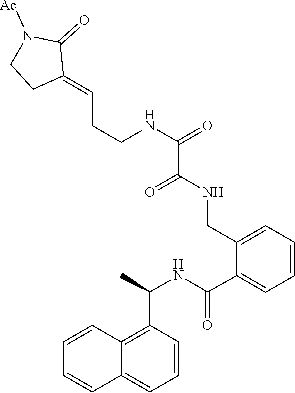 [SMILES: CC(=O)N1CC/C(=C\CCNC(=O)C(=O)NCc2ccccc2C(=O)N[C@H](C)c2cccc3ccccc23)C1=O] |
| 52a | 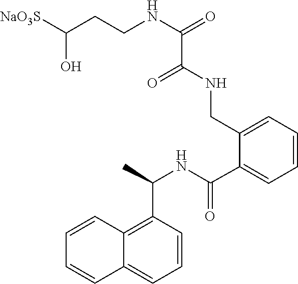 |
| 53a | 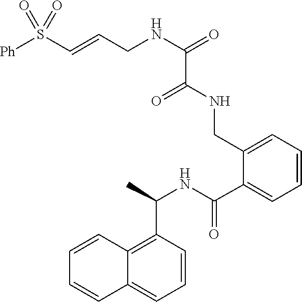 [SMILES: C[C@@H](NC(=O)c1ccccc1CNC(=O)C(=O)NC/C=C/S(=O)(=O)c1ccccc1)c1cccc2ccccc12] |
| 54a | 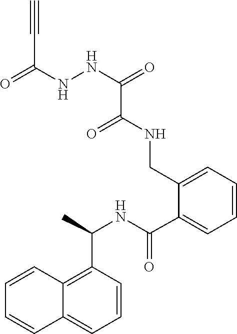 [SMILES: C#CC(=O)NNC(=O)C(=O)NCc1ccccc1C(=O)N[C@H](C)c1cccc2ccccc12] |
| 55a | 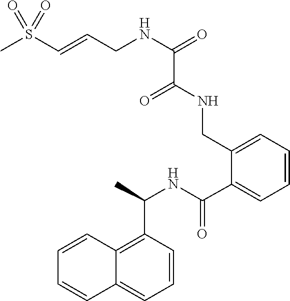 [SMILES: C[C@@H](NC(=O)c1ccccc1CNC(=O)C(=O)NC/C=C/S(C)(=O)=O)c1cccc2ccccc12] |
| 56a | 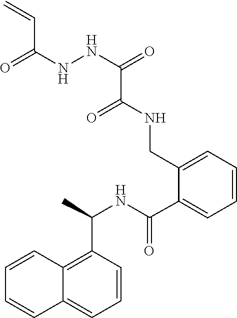 [SMILES: C=CC(=O)NNC(=O)C(=O)NCc1ccccc1C(=O)N[C@H](C)c1cccc2ccccc12] |
| 57a | 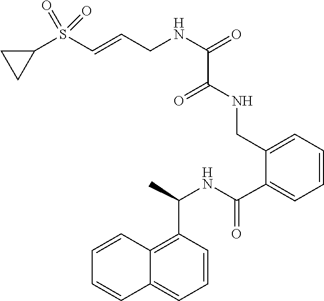 [SMILES: C[C@@H](NC(=O)c1ccccc1CNC(=O)C(=O)NC/C=C/S(=O)(=O)C1CC1)c1cccc2ccccc12] |
| 58a | 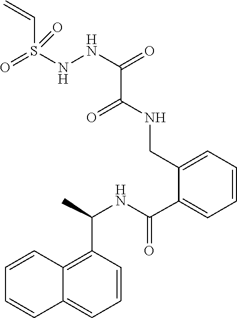 [SMILES: C=CS(=O)(=O)NNC(=O)C(=O)NCc1ccccc1C(=O)N[C@H](C)c1cccc2ccccc12] |
| 59a | 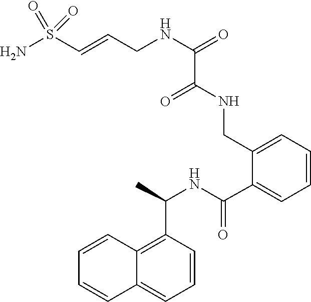 [SMILES: C[C@@H](NC(=O)c1ccccc1CNC(=O)C(=O)NC/C=C/S(N)(=O)=O)c1cccc2ccccc12] |
| 60a | 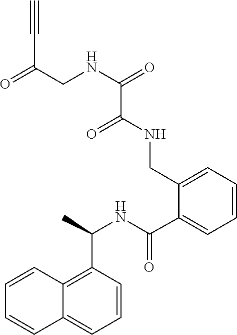 [SMILES: C#CC(=O)CNC(=O)C(=O)NCc1ccccc1C(=O)N[C@H](C)c1cccc2ccccc12] |
| 61a | 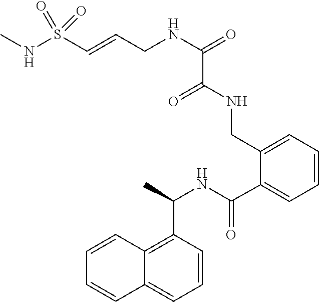 [SMILES: CNS(=O)(=O)/C=C/CNC(=O)C(=O)NCc1ccccc1C(=O)N[C@H](C)c1cccc2ccccc12] |
| 62a | 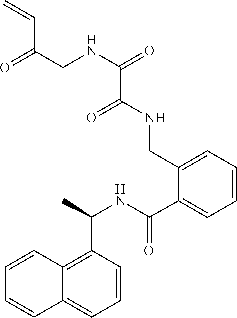 [SMILES: C=CC(=O)CNC(=O)C(=O)NCc1ccccc1C(=O)N[C@H](C)c1cccc2ccccc12] |
| 63a | 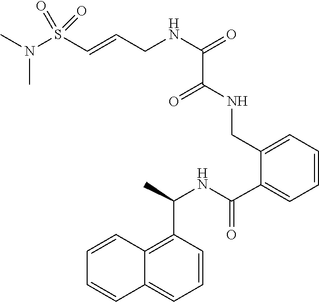 [SMILES: C[C@@H](NC(=O)c1ccccc1CNC(=O)C(=O)NC/C=C/S(=O)(=O)N(C)C)c1cccc2ccccc12] |
| 64a | 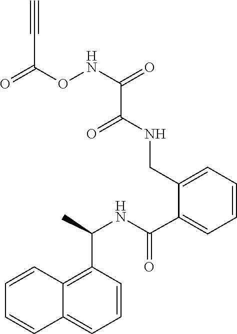 [SMILES: C#CC(=O)ONC(=O)C(=O)NCc1ccccc1C(=O)N[C@H](C)c1cccc2ccccc12] |
| 65a | 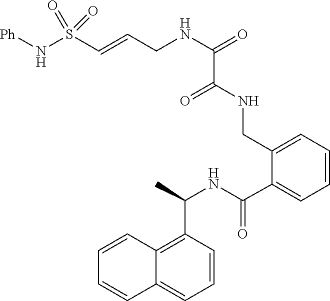 [SMILES: C[C@@H](NC(=O)c1ccccc1CNC(=O)C(=O)NC/C=C/S(=O)(=O)Nc1ccccc1)c1cccc2ccccc12] |
| 66a | 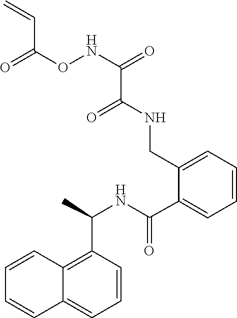 [SMILES: C=CC(=O)ONC(=O)C(=O)NCc1ccccc1C(=O)N[C@H](C)c1cccc2ccccc12] |
| 67a | 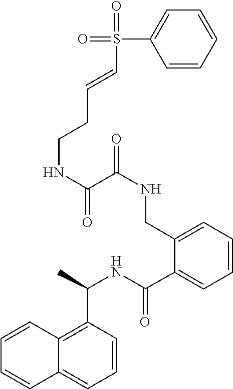 [SMILES: C[C@@H](NC(=O)c1ccccc1CNC(=O)C(=O)NCC/C=C/S(=O)(=O)c1ccccc1)c1cccc2ccccc12] |
| 68a | 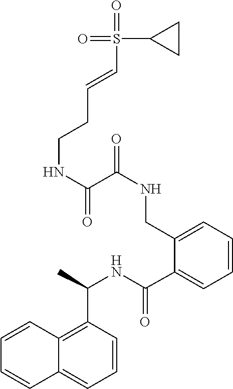 [SMILES: C[C@@H](NC(=O)c1ccccc1CNC(=O)C(=O)NCC/C=C/S(=O)(=O)C1CC1)c1cccc2ccccc12] |
| 69a | 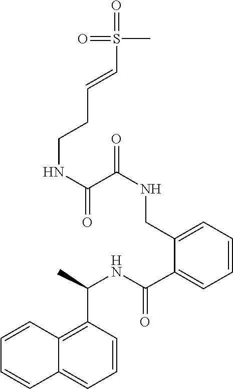 [SMILES: C[C@@H](NC(=O)c1ccccc1CNC(=O)C(=O)NCC/C=C/S(C)(=O)=O)c1cccc2ccccc12] |
| 70a | 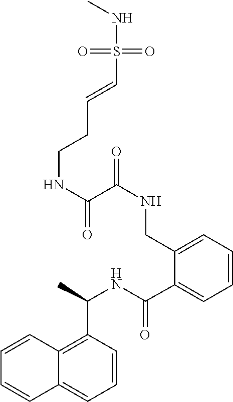 [SMILES: CNS(=O)(=O)/C=C/CCNC(=O)C(=O)NCc1ccccc1C(=O)N[C@H](C)c1cccc2ccccc12] |
| 71a | 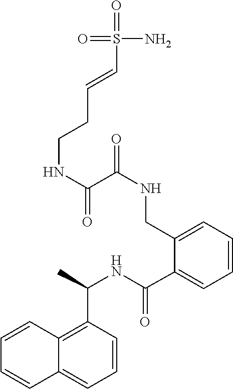 [SMILES: C[C@@H](NC(=O)c1ccccc1CNC(=O)C(=O)NCC/C=C/S(N)(=O)=O)c1cccc2ccccc12] |
| 72a | 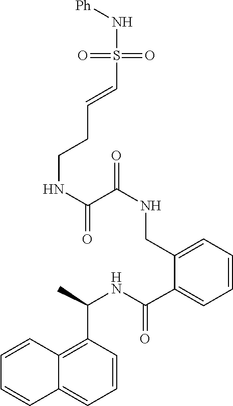 [SMILES: C[C@@H](NC(=O)c1ccccc1CNC(=O)C(=O)NCC/C=C/S(=O)(=O)Nc1ccccc1)c1cccc2ccccc12] |
| 73a | 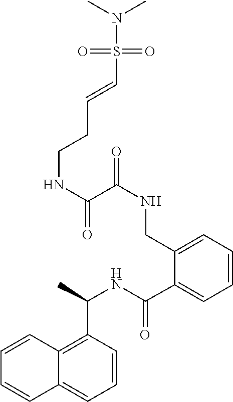 [SMILES: C[C@@H](NC(=O)c1ccccc1CNC(=O)C(=O)NCC/C=C/S(=O)(=O)N(C)C)c1cccc2ccccc12] |
| 74a | 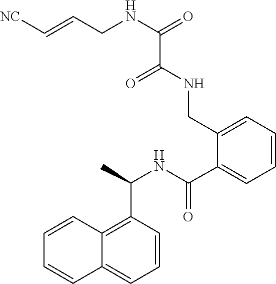 [SMILES: C[C@@H](NC(=O)c1ccccc1CNC(=O)C(=O)NC/C=C/C#N)c1cccc2ccccc12] |
| 75a | 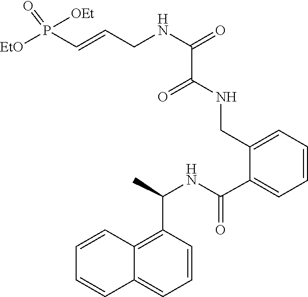 [SMILES: CCOP(=O)(/C=C/CNC(=O)C(=O)NCc1ccccc1C(=O)N[C@H](C)c1cccc2ccccc12)OCC] |
| 76a | 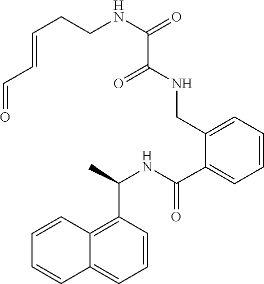 [SMILES: C[C@@H](NC(=O)c1ccccc1CNC(=O)C(=O)NCC/C=C/C=O)c1cccc2ccccc12] |
| 77a | 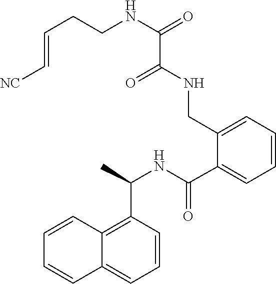 [SMILES: C[C@@H](NC(=O)c1ccccc1CNC(=O)C(=O)NCC/C=C/C#N)c1cccc2ccccc12] |
| 78a | 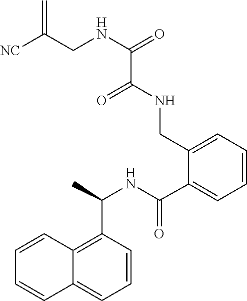 [SMILES: C=C(C#N)CNC(=O)C(=O)NCc1ccccc1C(=O)N[C@H](C)c1cccc2ccccc12] |
| 79a | 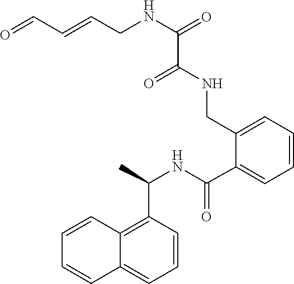 [SMILES: C[C@@H](NC(=O)c1ccccc1CNC(=O)C(=O)NC/C=C/C=O)c1cccc2ccccc12] |
| 80a | 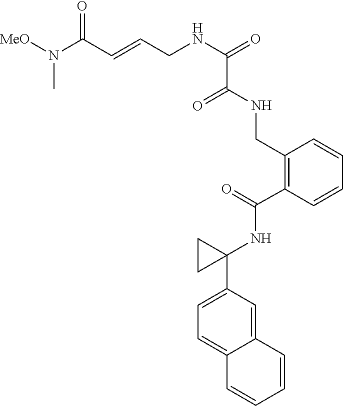 [SMILES: CON(C)C(=O)/C=C/CNC(=O)C(=O)NCc1ccccc1C(=O)NC1(c2ccc3ccccc3c2)CC1] |
| 81a | 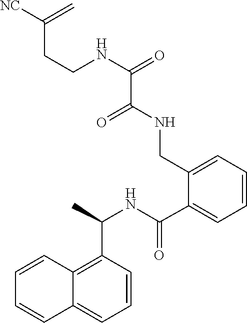 [SMILES: C=C(C#N)CCNC(=O)C(=O)NCc1ccccc1C(=O)N[C@H](C)c1cccc2ccccc12] |
| 82a | 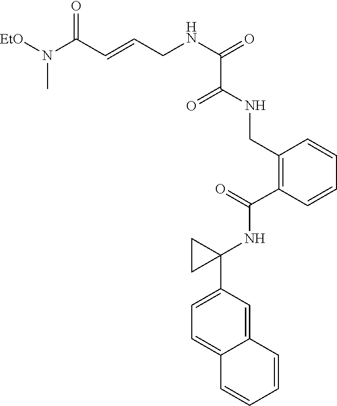 [SMILES: CCON(C)C(=O)/C=C/CNC(=O)C(=O)NCc1ccccc1C(=O)NC1(c2ccc3ccccc3c2)CC1] |
| 83a | 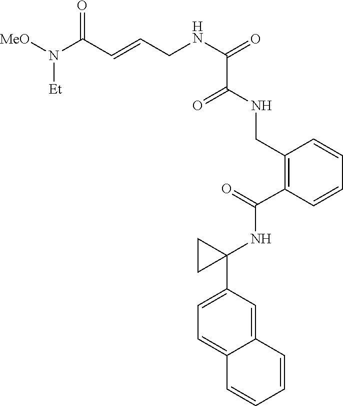 [SMILES: CCN(OC)C(=O)/C=C/CNC(=O)C(=O)NCc1ccccc1C(=O)NC1(c2ccc3ccccc3c2)CC1] |
| 84a | 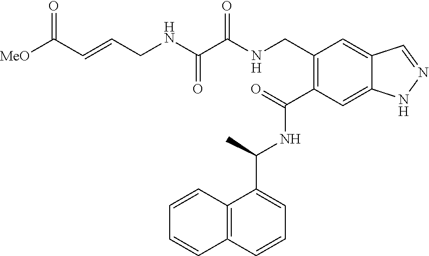 [SMILES: COC(=O)/C=C/CNC(=O)C(=O)NCc1cc2cn[nH]c2cc1C(=O)N[C@H](C)c1cccc2ccccc12] |
| 85a | 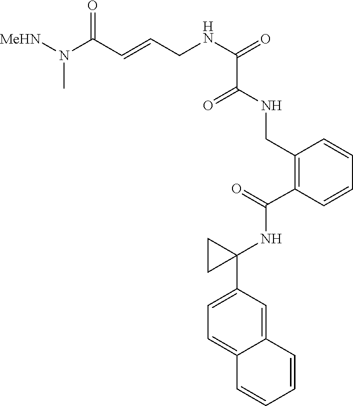 [SMILES: CNN(C)C(=O)/C=C/CNC(=O)C(=O)NCc1ccccc1C(=O)NC1(c2ccc3ccccc3c2)CC1] |
| 86a | 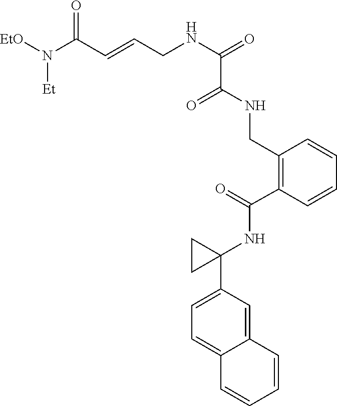 [SMILES: CCON(CC)C(=O)/C=C/CNC(=O)C(=O)NCc1ccccc1C(=O)NC1(c2ccc3ccccc3c2)CC1] |
| 87a | 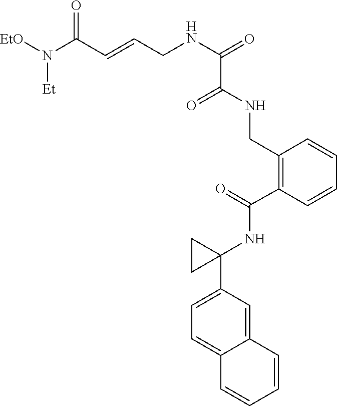 [SMILES: CCON(CC)C(=O)/C=C/CNC(=O)C(=O)NCc1ccccc1C(=O)NC1(c2ccc3ccccc3c2)CC1] |
| 88a | 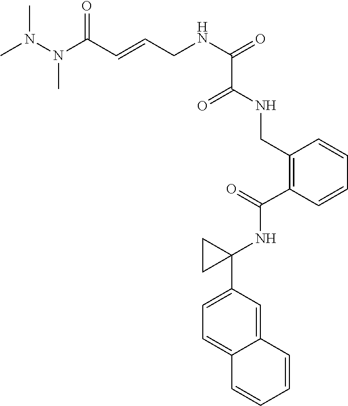 [SMILES: CN(C)N(C)C(=O)/C=C/CNC(=O)C(=O)NCc1ccccc1C(=O)NC1(c2ccc3ccccc3c2)CC1] |
| 89a | 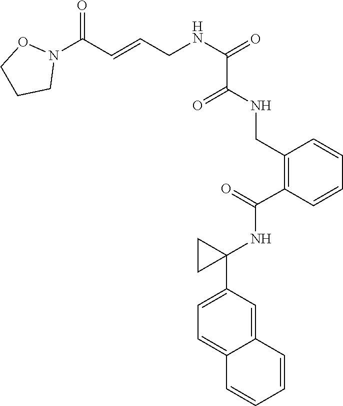 [SMILES: O=C(NC/C=C/C(=O)N1CCCO1)C(=O)NCc1ccccc1C(=O)NC1(c2ccc3ccccc3c2)CC1] |
| 90a | 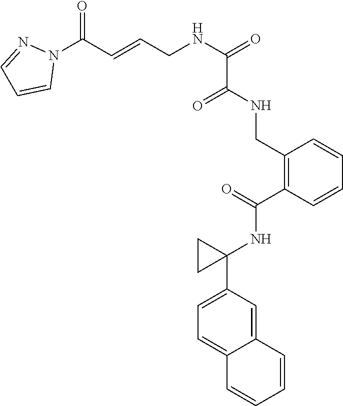 [SMILES: O=C(NC/C=C/C(=O)n1cccn1)C(=O)NCc1ccccc1C(=O)NC1(c2ccc3ccccc3c2)CC1] |
| 91a | 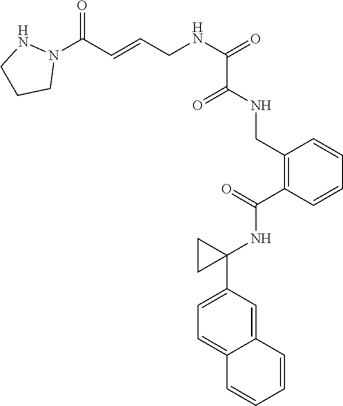 [SMILES: O=C(NC/C=C/C(=O)N1CCCN1)C(=O)NCc1ccccc1C(=O)NC1(c2ccc3ccccc3c2)CC1] |

### Biological Activity

SARS-CoV-2 Papain-like (PLpro) protease biochemical enzyme inhibition assay: Ubiquitin-modified at the C-terminal with a masked fluorophore is used as a substrate for PLpro which cleaves the fluorophore and the unmasked free fluorophore generates a fluorescent signal. The fluorophore is either Rhodamine or AMC. A similar assay is also explored using ISG15-AMC. Procedure: 1) Transfer compounds to 384w assay plates using Echo according to the general platemaps. 2) Add 10 μL of 1× Assay Buffer to LOW control wells and 10 μL of 2× PLpro solution to other wells in the assay plate according to the platemap, spin at 800 rpm for 1 min and incubate at RT for 30 mins. 3) Add 10 μL of 2× substrate solution to each well of enzyme solution according to platemap. Spin at 800 rpm for 1 min and incubate assay at RT in the dark for 30 mins. 4) Spin at 800 rpm and read plates on Virology Envision for Ub-R110 the Ex/Em was 485/535 nm. 5) Normalize data to high and low controls and determine IC50 by fitting data to normalized response versus inhibitor (variable slope) using GraphPad Prism 7. All experiments were run in duplicate, and IC₅₀ ranges are reported as follows: A<0.1 μM; B 0.1-1 μM; C 1-10 μM; D>10 μM.

SARS-CoV-2 Replicon Assay (Huh-7): A SARS-CoV-2 replicon expressing a Renilla luciferase reporter and rendered non-infectious due to deletions in the S, E, M, 3a, 3b, 6, 7a, 7b, and 8 open reading frames was utilized for evaluating compound activity against the autologous SARS-CoV-2 proteins in a cell-based assay. The SARS-CoV-2 replicon construct is a single bacterial artificial chromosome (BAC) encoded fragment (Codex DNA). The replicon fragment is amplified and linearized from the BAC by PCR using Platinum SuperFi II PCR Master Mix (Invitrogen) and the following primers: forward 5′-CGC ACG GTT ATG TGG ACC CTG-3′ (SEQ ID NO. 1) and reverse 5′-TTT TTT TTT TTT TTT TTT TTT TTT TTT TGT CAT TCT CCT AAG AAG CTA TTA-3′ (SEQ ID NO. 2). SARS-CoV-2 replicon RNA is synthesized by in vitro transcription using mMESSAGE mMACHINE T7 Ultra (Invitrogen). A plasmid encoding codon-optimized SARS-CoV-2 N is linearized by restriction digestion and used as a template for in vitro transcription using mMESSAGE mMACHINE T7 Ultra to produce SARS-CoV-2 N RNA. RNA is purified using the Monarch RNA cleanup kit (New England Biolabs). White 384-well tissue-culture-treated clear-bottom plates are used in this assay. Using a Labcyte ECHO liquid dispenser, 3-fold serial dilutions of compounds suspended in DMSO are added to the inner 308 wells in duplicate in a total volume of 125 nL per well. Two columns are treated only with 125 nL DMSO, to be used as controls. In a typical assay, 650 ng SARS-CoV-2 replicon RNA and 650 ng codon-optimized SARS-CoV-2 N RNA are mixed with 100 μL of HuH-7 cells suspended in Buffer R (Neon Transfection System, Invitrogen) at a final concentration of 1E7 cells/mL and co-electroporated using the Neon Transfection System at 1700V-20 ms-1 pulse. HuH-7 cells at 1E7 cells/mL without RNA are electroporated as a low control. For each 100 μL electroporated cell suspension, 1150 μL pre-warmed (37° C.) media (DMEM, 1× GlutaMAX, 10% FBS) is added to the cells for a final concentration of 800 cells/μL. The electroporated cells are seeded into the 308 inner compound and control wells of the 384-well plate at 20,000 cells per well in a total volume of 25 μL. One drug-free DMSO-treated column of wells is seeded with cells electroporated with replicon and N RNA as a high control. Another drug-free DMSO-treated column of wells is seeded with cells electroporated without RNA as a low control. The unused outer wells are filled with 25 μL of moat media containing 1% penicillin/streptomycin. Plates are incubated at 37° C. in a CO₂ humidity-controlled incubator for approximately 20 hours, then brought to room temperature. 25 μL of room temperature 1× Renilla-Glo Luciferase Assay Reagent (Promega) are added to each well and incubated at room temperature for 10 minutes and luminescence was measured on a Perkin Elmer EnVision. The Renilla-Glo reagent quantifies the amount of luciferase activity, which gives a measure of replicon activity present. The half-maximal effective concentration (EC₅₀) was determined using GraphPad Prism. Percent residual activity of the replicon is determined after normalizing the curve to the mean RLU high control at 100% and the mean RLU low control at 0%. EC₅₀ curves are generated using a variable slope four-parameter logistic model with equation Y=100/(1+X\hat{ }HillSlope)/(EC₅₀\hat{ }HillSlope). A <1 μM; B 1-10 μM; C>10 μM.

SARS-CoV-2 BSL3 Assay (Vero 76): Test compounds are serially diluted using eight half-log dilutions in test medium (MEM supplemented with 2% FBS and 50 pg/mL gentamicin). Each dilution is added to 5 wells of a 96-well plate with 80-100% confluent Vero 76 cells. Three wells of each dilution are infected with virus (SARS-CoV-2 USA-WA1/2020), and two wells remain uninfected as toxicity controls. Six wells are infected and untreated as virus controls, and six wells are uninfected and untreated as cell controls. Viruses are prepared to achieve the lowest possible multiplicity of infection (MOI˜0.002) that would yield >80% cytopathic effect (CPE) at 6 days. Plates are incubated at 37±2° C., 5% CO₂. For neutral red assay, on day 6 post-infection, once untreated virus control wells reach maximum CPE, plates are stained with neutral red dye for approximately 2 hours (±15 minutes). Supernatant dye is removed, and wells are rinsed with PBS, and the incorporated dye is extracted in 50:50 Sorensen citrate buffer/ethanol for >30 minutes and the optical density is read on a spectrophotometer at 540 nm. Optical densities are converted to percent of cell controls and normalized to the virus control, then the concentration of test compound required to inhibit CPE by 50% (EC₅₀) is calculated by regression analysis. The concentration of compound that would cause 50% cell death in the absence of virus was similarly calculated (CC₅₀). EC₅₀ ranges are reported as follows: A<1 μM; B 1-10 μM; C>10 μM.

TABLE 3
Summary of Activities

| | | SARS-CoV-2 | SARS-CoV-2 |
|---|---|---|---|
| Example # | PLpro IC₅₀ | replicon EC₅₀ | BSL3 EC₅₀ |
| 1 | A | A | A |
| 2 | A | A | A |
| 3 | A | A | A |
| 4 | A | A | |
| 5 | D | | |
| 6 | A | B | |
| 7 | D | | |
| 8 | A | A | |
| 9 | C | | |
| 10 | D | | |
| 11 | D | | |
| 12 | D | | |
| 13 | D | | |
| 14 | C | | |
| 15 | D | | |
| 16 | C | | |
| 17 | D | | |
| 18 | C | | |
| 19 | D | | |
| 20 | C | | |
| 21 | A | B | |
| 22 | B | C | |
| 23 | D | | |
| 24 | A | A | |
| 25 | A | A | |
| 26 | C | | |
| 27 | D | | |
| 28 | A | A | |
| 29 | A | B | |
| 30 | B | | |
| 31 | D | | |
| 32 | A | | |
| 33 | B | C | |
| 34 | A | | |
| 35 | A | | |
| 36 | D | | |
| 37 | B | | |
| 38 | A | | |
| 39 | B | | |
| 40 | A | | |
| 41 | D | | |
| 42 | D | | |
| 43 | A | | |
| 44 | A | | |
| 45 | C | | |
| 46 | A | | |
| 47 | B | | |
| 48 | D | | |
| 49 | D | | |
| 50 | D | | |
| 51 | D | | |
| 52 | C | | |
| 53 | A | A | |
| 54 | A | A | |
| 55 | A | B | |
| 56 | A | A | |
| 57 | C | | |
| 58 | D | | |
| 59 | A | A | |
| 60 | B | | |
| 61 | A | A | |
| 62 | A | B | |
| 63 | D | | |
| 64 | D | | |
| 65 | D | | |
| 66 | D | | |
| 67 | D | | |
| 68 | D | | |
| 69 | A | A | |
| 70 | B | A | |
| 71 | A | A | |
| 72 | B | | |
| 73 | A | A | |
| 74 | A | B | |
| 75 | D | | |
| 76 | D | | |
| 77 | A | B | |
| 78 | B | | |
| 79 | A | A | |
| 80 | D | | |
| 81 | A | A | |
| 82 | A | A | |
| 83 | A | A | |
| 84 | A | A | |
| 85 | A | A | |
| 86 | A | A | |
| 87 | A | A | |
| 88 | A | A | |
| 89 | D | | |
| 90 | A | B | |
| 91 | D | | |
| 92 | C | | |
| 93 | D | | |
| 94 | A | A | |
| 95 | B | | |
| 96 | D | | |
| 97 | B | A | |
| 98 | A | A | |
| 99 | A | A | |
| 100 | A | A | |
| 101 | B | | |
| 102 | D | | |
| 103 | A | A | |
| 104 | A | A | |
| 105 | A | A | |
| 106 | A | A | |
| 107 | A | A | |
| 108 | D | | |
| 109 | A | A | |
| 110 | C | | |
| 111 | C | | |
| 112 | A | | |
| 113 | A | | |
| 114 | A | A | |
| 115 | A | A | |
| 116 | A | | |
| 117 | A | B | |
| 118 | D | | |
| 119 | C | | |
| 120 | D | | |
| 121 | A | A | |
| 122 | D | | |
| 123 | D | | |
| 124 | C | | |
| 125 | A | B | A |
| 126 | A | A | A |
| 127 | A | B | |
| 128 | A | A | |
| 129 | A | A | |
| 130 | A | B | |
| 131 | A | A | |
| 132 | A | A | |
| 133 | A | B | |
| 134 | A | A | |
| 135 | A | A | |
| 136 | A | A | |
| 137 | A | A | |
| 138 | A | A | |
| 139 | A | A | |
| 140 | A | A | |
| 141 | B | | |
| 142 | B | | |
| 143 | C | | |
| 144 | A | A | |
| 145 | A | A | |
| 146 | A | A | |
| 147 | A | A | |
| 148 | A | | |
| 149 | A | A | |
| 150 | C | | |
| 151 | A | A | |
| 152 | A | A | |
| 153 | A | A | |
| 154 | A | A | |
| 155 | A | A | |
| 156 | A | A | |
| 157 | C | — | |
| 158 | A | A | |
| 159 | A | A | |
| 160 | A | A | |
| 161 | A | A | |
| 162 | B | — | |
| 163 | A | A | |
| 164 | C | — | |
| 165 | A | B | |
| 166 | A | A | |
| 167 | A | A | |
| 168 | A | A | |
| 169 | D | — | |
| 170 | C | — | |
| 171 | A | A | |
| 172 | A | A | |
| 173 | A | A | |
| 174 | A | A | |
| 175 | B | B | |
| 176 | A | A | |
| 177 | B | B | |
| 178 | A | A | |
| 179 | A | A | |
| 180 | C | B | |
| 181 | A | A | |
| 182 | D | — | |
| 183 | A | A | |
| 184 | A | A | |
| 185 | A | A | |
| 186 | A | A | |
| 187 | A | A | |
| 188 | A | A | |
| 189 | A | A | |
| 190 | A | A | |
| 191 | A | A | |
| 192 | A | A | |
| 193 | A | A | |
| 194 | A | A | |
| 195 | A | A | |
| 196 | A | A | |
| 197 | A | A | |
| 198 | A | A | |
| 199 | A | A | |
| 200 | A | A | |
| 201 | A | A | |
| 202 | D | — | |
| 203 | D | — | |
| 204 | D | — | |
| 205 | D | — | |
| 206 | A | A | |
| 207 | D | — | |
| 208 | D | — | |
| 209 | A | A | |
| 210 | — | — | |
| 211 | A | A | |
| 212 | B | B | |
| 213 | A | A | |
| 214 | A | A | |
| 215 | A | A | |
| 216 | A | A | |
| 217 | B | C | |
| 218 | B | C | |
| 219 | A | B | |
| 220 | A | B | |
| 221 | A | B | |
| 222 | A | C | |
| 223 | A | A | |
| 224 | A | A | |
| 225 | A | A | |
| 226 | A | A | |
| 227 | A | A | |
| 228 | A | A | |
| 229 | A | B | |
| 230 | A | A | |
| 231 | A | C | |
| 232 | A | A | |
| 233 | A | C | |
| 234 | A | A | |
| 235 | A | C | |
| 236 | B | C | |
| 237 | A | A | |
| 238 | A | A | |
| 239 | A | C | |
| 240 | A | C | |
| 241 | A | B | |
| 242 | A | A | |
| 243 | A | A | |
| 244 | A | A | |
| 245 | A | A | |
| 246 | A | A | |
| 247 | A | A | |
| 248 | A | A | |
| 249 | A | A | |
| 250 | A | A | |
| 251 | A | A | |
| 252 | A | A | |
| 253 | A | A | |
| 254 | A | A | |
| 255 | A | A | |
| 256 | A | — | |
| 257 | A | A | |
| 258 | A | A | |
| 259 | A | A | |
| 260 | A | A | |
| 261 | A | A | |
| 262 | A | A | |
| 263 | A | A | |
| 264 | A | A | |
| 265 | A | A | |
| 266 | A | A | |
| 267 | A | A | |
| 268 | B | — | |
| 269 | A | A | |
| 270 | A | A | |
| 271 | A | A | |
| 272 | A | A | |
| 273 | A | A | |
| 274 | A | A | |
| 275 | A | A | |
| 276 | A | A | |
| 277 | A | A | |
| 278 | B | — | |
| 279 | A | A | |
| 280 | A | A | |
| 281 | A | A | |
| 282 | B | — | |
| 283 | A | A | |
| 284 | A | A | |
| 285 | A | A | |
| 286 | A | A | |
| 287 | A | A | |
| 288 | A | B | |
| 289 | A | A | |
| 290 | A | A | |
| 291 | D | — | |
| 292 | A | B | |
| 293 | A | A | |
| 294 | A | A | |
| 295 | A | A | |
| 296 | A | A | |
| 297 | A | A | |
| 298 | A | A | |
| 299 | A | A | |
| 300 | A | A | |
| 301 | A | A | |
| 302 | A | A | |
| 303 | A | A | |
| 304 | A | B | |
| 305 | A | A | |
| 306 | A | A | |
| 307 | A | B | |
| 308 | A | A | |
| 309 | C | — | |
| 310 | A | A | |
| 311 | A | A | |
| 312 | A | B | |
| 313 | A | A | |
| 314 | B | B | |
| 315 | C | — | |
| 316 | B | — | |
| 317 | A | — | |
| 318 | A | A | |
| 319 | A | B | |
| 320 | A | A | |
| 321 | A | B | |
| 322 | A | A | |
| 323 | A | A | |
| 324 | A | B | |
| 325 | D | — | |
| 326 | A | B | |
| 327 | A | B | |
| 328 | A | B | |
| 329 | A | A | |
| 330 | A | B | |
| 331 | A | A | |
| 332 | A | — | |
| 333 | A | A | |
| 334 | A | A | |
| 335 | A | A | |
| 336 | A | A | |
| 337 | A | A | |
| 338 | A | A | |
| 339 | A | | |
| 340 | B | — | |
| 341 | B | C | |
| 342 | A | A | |
| 343 | A | A | |
| 344 | A | A | |
| 345 | A | A | |
| 346 | A | A | |
| 347 | A | A | |
| 348 | A | B | |
| 349 | A | A | |
| 350 | A | A | |
| 351 | A | A | |
| 352 | A | A | |
| 353 | A | A | |
| 354 | A | A | |
| 355 | A | A | |
| 356 | A | B | |
| 357 | A | A | |
| 358 | A | A | |
| 359 | A | B | |
| 360 | B | B | |
| 361 | D | — | |
| 362 | A | A | |
| 363 | B | A | |
| 364 | D | — | |
| 365 | D | — | |
| 366 | A | B | |
| 367 | A | A | A |
| 368 | D | — | |
| 369 | A | B | |
| 370 | B | B | |
| 371 | C | — | |
| 372 | D | — | |
| 373 | B | — | |
| 374 | A | — | |
| 375 | C | — | |
| 376 | B | — | |
| 377 | B | — | |
| 378 | B | — | |
| 379 | A | — | |
| 380 | B | C | |
| 381 | A | A | |
| 382 | A | B | |
| 383 | A | A | |
| 384 | A | A | |
| 385 | A | A | |
| 386 | A | A | |
| 387 | A | A | |
| 388 | A | — | |
| 389 | A | — | |
| 390 | A | — | |
| 391 | A | — | |
| 392 | A | B | |
| 393 | A | B | |
| 394 | A | A | |
| 395 | B | — | |
| 396 | A | A | |
| 397 | B | — | |
| 398 | A | A | |
| 399 | A | A | |
| 400 | A | — | |
| 401 | C | — | |
| 402 | A | A | |
| 403 | C | — | |
| 404 | A | A | |
| 405 | B | — | |
| 406 | A | — | |
| 407 | A | — | |
| 408 | A | — | |
| 409 | A | B | |
| 410 | B | — | |
| 411 | A | B | |
| 412 | C | — | |
| 413 | A | A | |
| 414 | B | — | |
| 415 | A | B | |
| 416 | — | A | |
| 417 | — | A | |
| 418 | A | A | |
| 419 | A | A | |
| 420 | C | — | |
| 421 | C | — | |
| 422 | A | — | |
| 423 | A | B | |
| 424 | A | B | |
| 425 | A | A | |
| 426 | A | B | |
| 427 | A | A | |
| 428 | A | B | |
| 429 | A | A | |
| 430 | B | — | |
| 431 | B | B | |
| 432 | B | B | |
| 433 | C | — | |
| 434 | A | B | |
| 435 | A | A | |
| 436 | B | — | |
| 437 | B | — | |
| 438 | A | A | |
| 439 | A | A | |
| 440 | A | A | |
| 441 | A | A | |
| 442 | A | A | |
| 443 | A | A | |
| 444 | A | A | |
| 445 | A | B | |
| 446 | A | A | |
| 447 | A | A | |
| 448 | A | B | |
| 449 | A | A | |
| 450 | A | A | |
| 451 | A | B | |
| 452 | A | B | |
| 453 | A | A | |
| 454 | A | B | |
| 455 | B | C | |
| 456 | A | B | |
| 457 | A | B | B |
| 458 | A | A | |
| 459 | A | B | |
| 460 | A | A | |
| 461 | A | A | |
| 462 | A | A | |
| 463 | B | B | |
| 464 | A | B | |
| 465 | A | A | |
| 466 | D | — | |
| 467 | A | A | |
| 468 | B | — | |
| 469 | A | B | |
| 470 | A | B | |
| 471 | A | A | |
| 472 | B | C | |
| 473 | B | B | |
| 474 | B | — | |
| 475 | B | | |
| 476 | B | B | |
| 477 | A | A | |
| 478 | B | — | |
| 479 | B | — | |
| 480 | A | B | |
| 481 | B | B | |
| 482 | A | — | |
| 483 | B | — | |
| 484 | A | A | |
| 485 | A | A | |
| 486 | A | A | |
| 487 | A | A | |
| 488 | A | — | |
| 489 | A | A | |
| 490 | A | A | |
| 491 | A | A | |
| 492 | A | A | |
| 493 | A | A | |
| 494 | A | A | |
| 495 | A | A | |
| 496 | A | A | |
| 497 | D | — | |
| 498 | B | — | |
| 499 | C | — | |
| 500 | A | A | |
| 501 | C | — | |
| 502 | B | — | |
| 503 | C | — | |
| 504 | A | A | |
| 505 | C | — | |
| 506 | D | — | |
| 507 | D | — | |
| 508 | B | — | |
| 509 | A | A | |
| 510 | — | — | |
| 511 | C | — | |
| 512 | C | | |
| 513 | D | | |
| 514 | — | | |
| 515 | — | | |
| 516 | A | | |
| 517 | — | | |
| 518 | A | A | A |
| 519 | A | A | |
| 520 | A | A | |
| 521 | B | | |
| 522 | B | | |
| 523 | D | | |
| 524 | A | A | |
| 525 | A | A | B |
| 526 | A | A | |
| 527 | A | A | A |
| 528 | A | A | A |
| 529 | A | A | A |
| 530 | B | | |
| 531 | A | | |
| 532 | A | A | |
| 533 | B | | |
| 534 | B | | |
| 535 | A | A | B |
| 536 | A | A | A |
| 537 | A | A | A |
| 538 | B | B | |
| 539 | B | A | |
| 540 | D | | |
| 541 | B | C | |
| 542 | D | | |
| 543 | B | C | |
| 544 | C | | |
| 545 | B | C | |
| 546 | C | | |
| 547 | D | | |
| 548 | B | | |
| 549 | B | | |
| 550 | D | | |
| 551 | C | | |
| 552 | B | | |
| 553 | B | | |
| 554 | B | B | |
| 555 | B | B | |
| 556 | B | B | |
| 557 | A | B | |
| 558 | B | B | |
| 559 | B | B | |
| 560 | B | B | |
| 561 | B | B | |
| 562 | D | | |
| 563 | A | C | |
| 564 | B | B | |
| 565 | C | | |
| 566 | B | B | |
| 567 | B | B | |
| 568 | B | B | |
| 569 | B | A | |
| 571 | B | B | |
| 572 | B | C | |
| 573 | B | B | |
| 574 | B | B | |
| 575 | B | C | |
| 576 | A | A | |
| 577 | A | A | |
| 578 | B | | |
| 579 | B | | |
| 580 | B | | |
| 581 | B | | |
| 582 | A | A | |
| 583 | B | | |

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

1. A compound represented by Formula (I), or a pharmaceutically acceptable salt thereof, 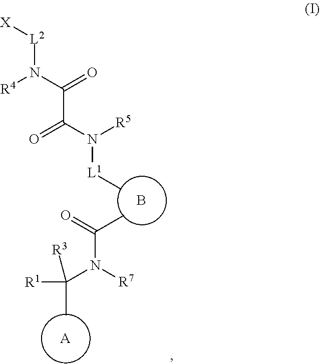
wherein
A is selected from the group consisting of optionally substituted aryl, optionally substituted heteroaryl, optionally substituted C₃-C₈ cycloalkyl, and optionally substituted 3- to 8-membered heterocycloalkyl;
B is selected from the group consisting of optionally substituted aryl, optionally substituted heteroaryl, optionally substituted C₃-C₈ cycloalkyl, and optionally substituted 3- to 8-membered heterocycloalkyl;
R¹ and R³ are each independently selected from the group consisting of hydrogen, optionally substituted C₁-C₄ alkyl, optionally substituted C₂-C₄ alkenyl, optionally substituted C₂-C₄ alkynyl, and optionally substituted C₃-C₆ cycloalkyl; alternatively, R¹ and R³ are taken together with the carbon atom to which they are attached to form an optionally substituted 3- to 8-membered carbocyclic or 3- to 8-membered heterocyclic ring;
R⁴, R⁵, and R⁷ are each independently selected from the group consisting of hydrogen, optionally substituted C₁-C₆ alkyl, optionally substituted C₂-C₆ alkenyl, optionally substituted C₂-C₆ alkynyl, optionally substituted C₃-C₈ cycloalkyl, optionally substituted 3- to 8-membered heterocycloalkyl, optionally substituted aryl, and optionally substituted heteroaryl;
L¹ and L² are each independently selected from the group consisting of —(CR²¹R²³)_{q}—, —CR²¹═CR²², —C≡C—, —(CR²¹R²³)_{q}—O—, —(CR²¹R²³)_{q}—S—, —(CR²¹R²³)_{q}—NR¹²—, 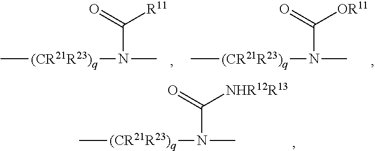
—(CR²¹R²³)_{q}—NR¹²C(O)—, —(CR²¹R²³)_{q}—NR¹²C(O)O—, —(CR²¹R²³)_{q}—NR¹²C(O)NR¹³—, —(CR²¹R²³)_{q}—C(O)N(R¹²)—, —(CR²¹R²³)_{q}— N(R¹²)C(O)—, —(CR²¹R²³)_{q}—C(O)—, —(CR²¹R²³)_{q}—OC(O)—, —(CR²¹R²³)_{q}—S(O)₂—, —(CR²¹R²³)_{q}—S(O)—, —(CR²¹R²³)_{q}—S(O)(NR¹²)—, —(CR²¹R²³)_{q}—(NR¹²)S(O)—, —(CR²¹R²³)_{q}—S(O)₂NR¹²—, —(CR²¹R²³)_{q}— NR¹²S(O)₂—, optionally substituted C₃-C₁₂ cycloalkyl, optionally substituted C₃-C₁₂ cycloalkenyl, optionally substituted 3- to 12-membered heterocycloalkyl, optionally substituted aryl, optionally substituted arylalkyl, optionally substituted heteroaryl, and optionally substituted heteroarylalkyl;
L¹ connects to B through a carbon, nitrogen, sulfur, or oxygen atom;
q is 0, 1, 2, 3 or 4;
each R¹¹ is independently selected from the group consisting of hydrogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
R¹² and R¹³ at each occurrence are independently selected from the group consisting of hydrogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
alternatively R¹² and R¹³ are taken together with the nitrogen atom to which they are attached to form an optionally substituted 3- to 8-membered heterocyclic ring;
R²¹ and R²² at each occurrence are independently selected from the group consisting of hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl; in certain embodiments, R²¹ and R²² are both hydrogen;
R²³ at each occurrence is independently selected from the group consisting of hydrogen, halogen, —OR¹¹, —OC(O)R¹¹, —OC(O)OR¹¹, —OC(O)NR¹²R¹³, —NR¹²R¹³, —NR¹²C(O)R¹¹, —NR¹²C(O)OR¹³, —NR₁₂C(O)NR¹²R¹³, —C(O)NR¹²R¹³, —N₃, —CN, optionally substituted C₁-C₈ alkyl, optionally substituted C₂-C₈ alkenyl, optionally substituted C₂-C₈ alkynyl, optionally substituted C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
alternatively, R⁴ and a substituent of L² are taken together with the intervening atoms to form 3- to 8-membered heterocyclic ring;
X is selected from the group consisting of hydrogen, halogen, —CN, —C(O)R²⁵, —CH(OH)SO₃R²⁶, —C(O)NR²⁷R²⁸, —C(O)OR²⁷, —C(O)C(O)OR²⁷, —C(O)C(O)NR²⁷R²⁸, —C(O)(CR²¹R²³)C(O)OR²⁷, —C(O)(CR²¹R²³)C(O)NR²⁷R²⁸, —C(O)S(O)₂NR²⁷R²⁸, —C(O)S(O)NR²⁷R²⁸, 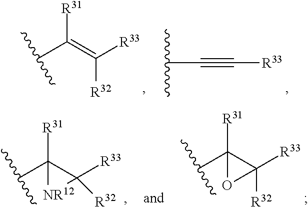
R²⁵ is hydrogen, halogen, hydroxy, or optionally substituted C₁-C₈ alkyl;
R²⁶ is hydrogen or Na⁺;
R²⁷ and R²⁸ at each occurrence are each independently selected from the group consisting of hydrogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted 3- to 8-membered heterocycloalkyl, optionally substituted aryl, optionally substituted arylalkyl, optionally substituted heteroaryl, and optionally substituted heteroarylalkyl;
alternatively, R²⁷ and R²⁸ are taken together with the nitrogen atom to which they are attached to form an optionally substituted 3- to 8-membered heterocyclic ring;
R³¹, R³² and R³³ are each independently selected from the group consisting of hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted 3- to 8-membered heterocycloalkyl, optionally substituted aryl, optionally substituted heteroaryl, —CN, —NO₂, —NO, —C(O)R²¹, —CH(OH)SO₃R²⁶; —C(O)NR²⁷R²⁸, —C(O)OR²⁷, —C(O)SR²⁷, —S(O)₂R²⁷, —S(O)₂OR²⁷, —S(O)R²⁷, —S(O)OR²⁷, —S(O)₂NR²⁷R²⁸, —S(O)NR²⁷R²¹, —P(O)R²⁷R²⁸, —P(O)OR²⁷R²⁸, —P(O)OR²⁷OR²⁸, —P(O)NR¹²R²⁷R²⁸, —P(O)NR¹²R¹³NR²⁷R²⁸, —P(O)NR¹²R¹³OR²⁸, —C(O)C(O)NR²⁷R²⁸, —C(O)C(O)OR²⁷, —C(O)S(O)₂NR²⁷R²⁸, and —C(O)S(O)NR²⁷R²⁸;
alternatively, R³¹ and R³³ are taken together with the carbon atoms to which they are attached to form an optionally substituted C₄-C₈ cycloalkyl, C₄-C₈ cycloalkenyl or optionally substituted C₄-C₈ cycloalkenyl or 4- to 8-membered heterocyclic ring; alternatively, R³² and R³³ are taken together with the carbon atom to which they are attached to form an optionally substituted C₃-C₈ cycloalkyl or optionally substituted 3- to 8-membered heterocyclic ring;
alternatively, R³¹ and L² are taken together with the nitrogen, carbon, sulfur, or oxygen atom to which they are attached to form an optionally substituted 3- to 8-membered heterocyclic ring; and
alternatively, R³² and a substituent of L² are taken together with the intervening atoms to form an optionally substituted 4- to 8-membered heterocyclic ring.

2. The compound of claim 1, wherein A is derived from one of the following by removal of one hydrogen atom and A is optionally substituted:

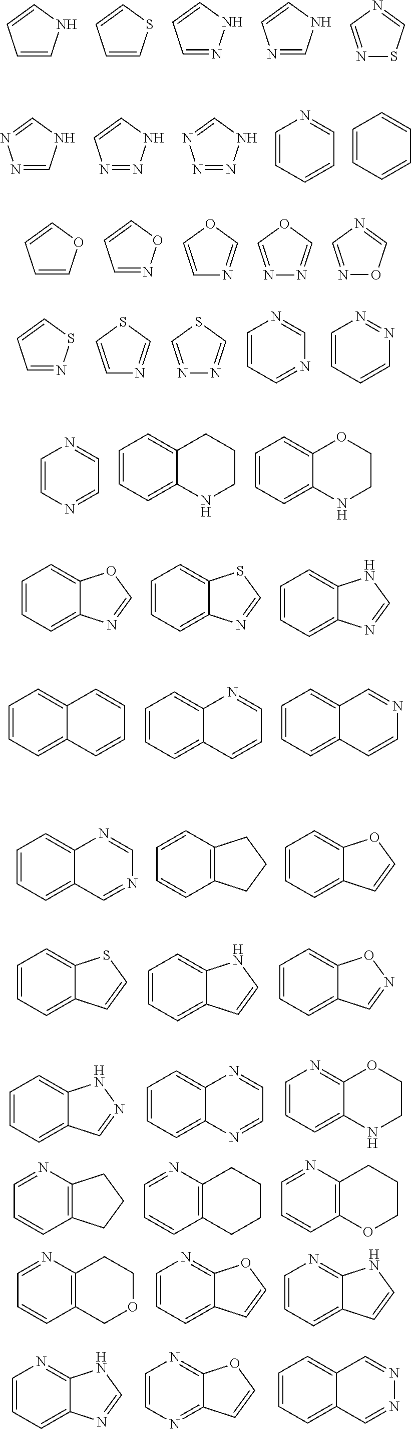

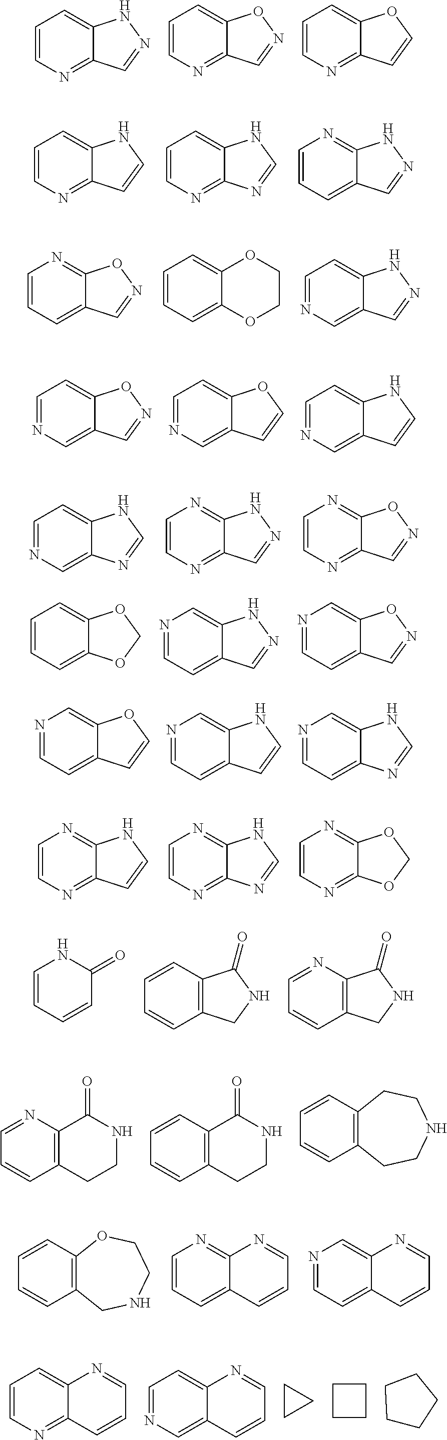

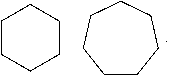

3. The compound of claim 1, B is optionally substituted phenyl.

4. The compound of claim 1, represented by Formula (V-1) or Formula (V-2), or a pharmaceutically acceptable salt thereof: 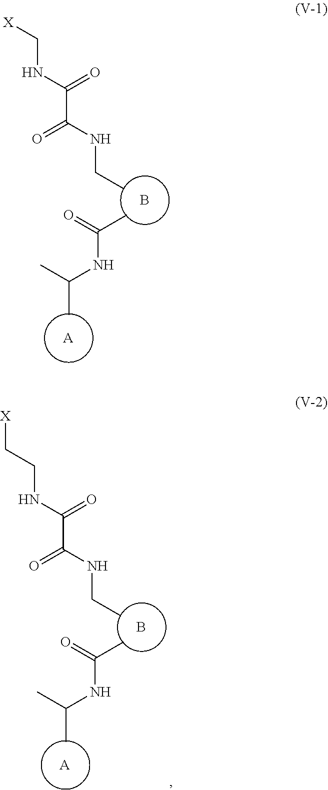
wherein A, B, and X are as defined in claim 1.

5. The compound of claim 1, represented by Formula (XV-1) or Formula (XV-2), or a pharmaceutically acceptable salt thereof: 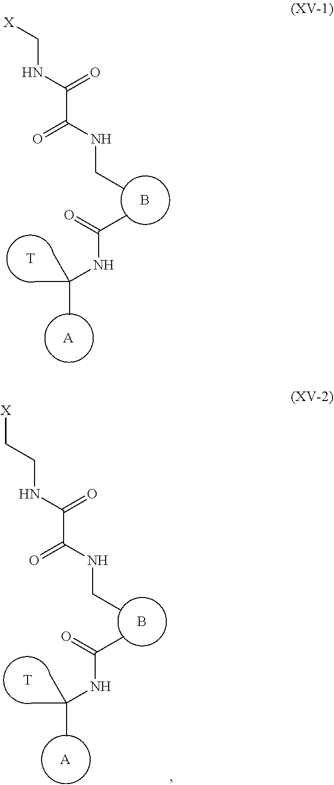
wherein T is selected from the group consisting of optionally substituted C₃-C₈ cycloalkyl, optionally substituted 3- to 8-membered heterocycloalkyl, and optionally substituted C₃-C₈ cycloalkenyl; A, B, and X are as defined in claim 1.

6. The compound of claim 1, represented by one of Formulas (XXIII-1)˜(XXIII-4), or a pharmaceutically acceptable salt thereof: 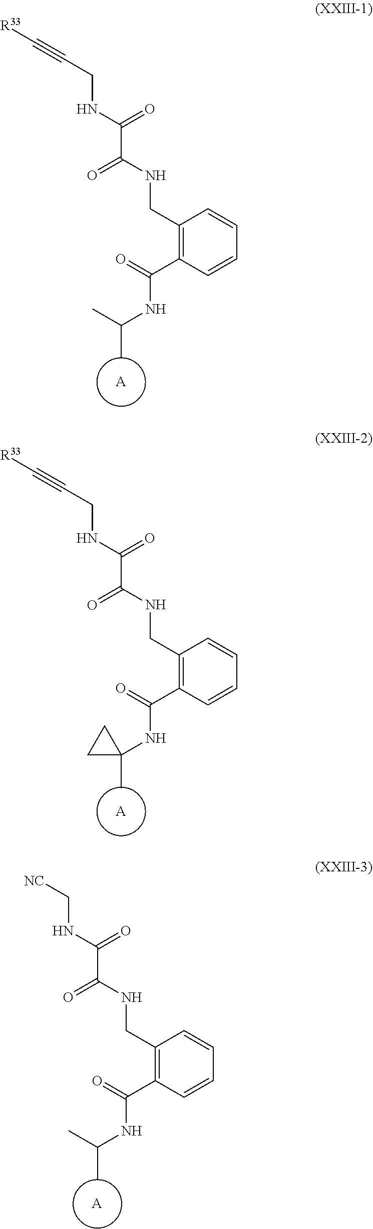 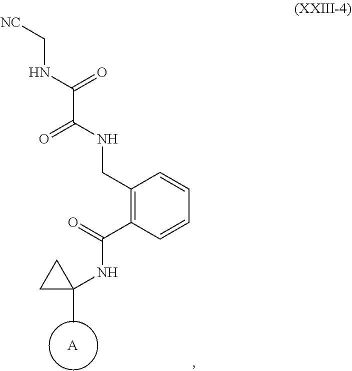
wherein A and R³³ are as defined in claim 1.

7. The compound of claim 1, selected from the compounds set forth below or a pharmaceutically acceptable salt thereof:

| Compound | Structure |
|---|---|
| 1 | 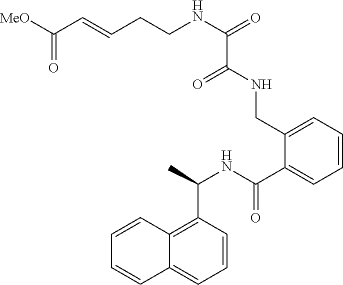 [SMILES: COC(=O)/C=C/CCNC(=O)C(=O)NCc1ccccc1C(=O)N[C@H](C)c1cccc2ccccc12] |
| 2 | 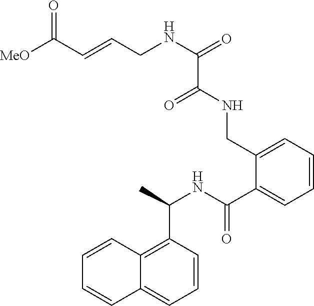 [SMILES: COC(=O)/C=C/CNC(=O)C(=O)NCc1ccccc1C(=O)N[C@H](C)c1cccc2ccccc12] |
| 3 | 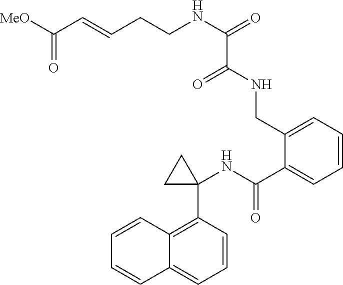 [SMILES: COC(=O)/C=C/CCNC(=O)C(=O)NCc1ccccc1C(=O)NC1(c2cccc3ccccc23)CC1] |
| 4 | 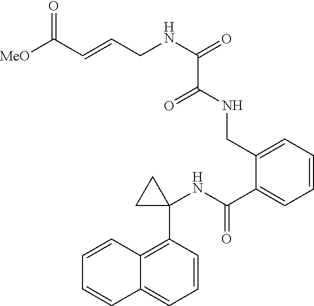 [SMILES: COC(=O)/C=C/CNC(=O)C(=O)NCc1ccccc1C(=O)NC1(c2cccc3ccccc23)CC1] |
| 5 | 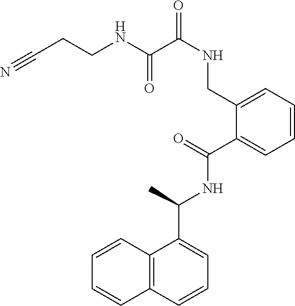 [SMILES: C[C@@H](NC(=O)c1ccccc1CNC(=O)C(=O)NCCC#N)c1cccc2ccccc12] |
| 6 | 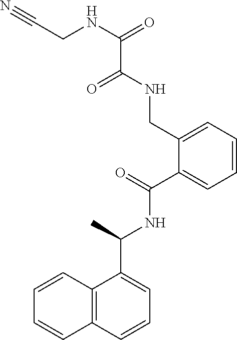 [SMILES: C[C@@H](NC(=O)c1ccccc1CNC(=O)C(=O)NCC#N)c1cccc2ccccc12] |
| 7 | 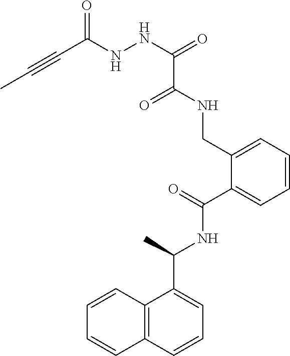 [SMILES: CC#CC(=O)NNC(=O)C(=O)NCc1ccccc1C(=O)N[C@H](C)c1cccc2ccccc12] |
| 8 | 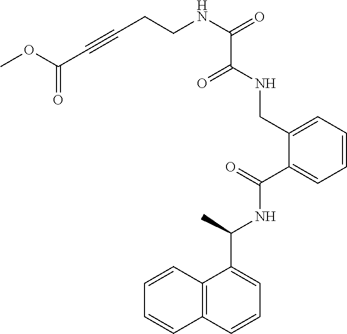 [SMILES: COC(=O)C#CCCNC(=O)C(=O)NCc1ccccc1C(=O)N[C@H](C)c1cccc2ccccc12] |
| 9 | 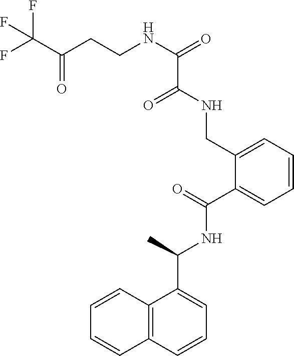 [SMILES: C[C@@H](NC(=O)c1ccccc1CNC(=O)C(=O)NCCC(=O)C(F)(F)F)c1cccc2ccccc12] |
| 10 | 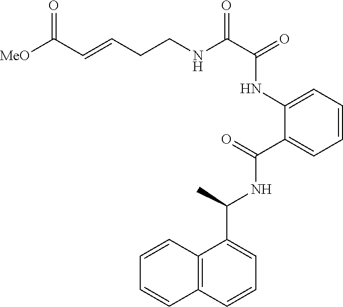 [SMILES: COC(=O)/C=C/CCNC(=O)C(=O)Nc1ccccc1C(=O)N[C@H](C)c1cccc2ccccc12] |
| 11 | 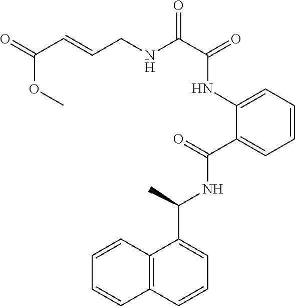 [SMILES: COC(=O)/C=C/CNC(=O)C(=O)Nc1ccccc1C(=O)N[C@H](C)c1cccc2ccccc12] |
| 12 | 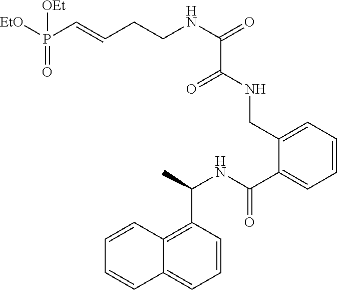 [SMILES: CCOP(=O)(/C=C/CCNC(=O)C(=O)NCc1ccccc1C(=O)N[C@H](C)c1cccc2ccccc12)OCC] |
| 13 | 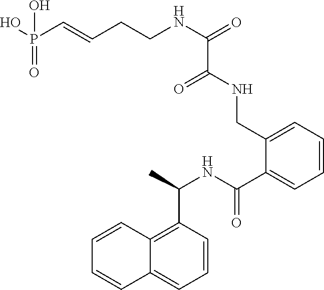 [SMILES: C[C@@H](NC(=O)c1ccccc1CNC(=O)C(=O)NCC/C=C/P(=O)(O)O)c1cccc2ccccc12] |
| 14 | 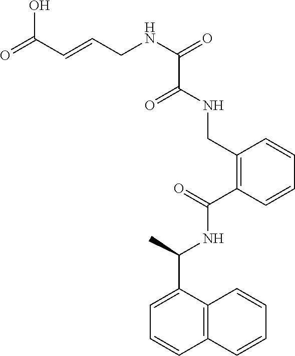 [SMILES: C[C@@H](NC(=O)c1ccccc1CNC(=O)C(=O)NC/C=C/C(=O)O)c1cccc2ccccc12] |
| 15 | 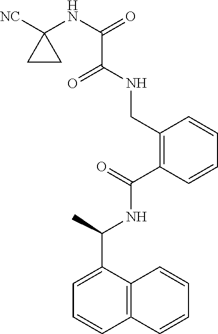 [SMILES: C[C@@H](NC(=O)c1ccccc1CNC(=O)C(=O)NC1(C#N)CC1)c1cccc2ccccc12] |
| 16 | 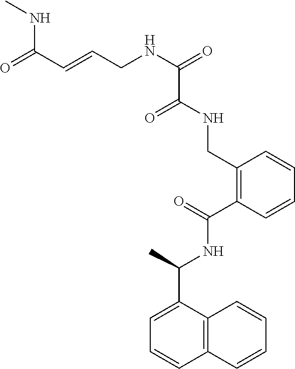 [SMILES: CNC(=O)/C=C/CNC(=O)C(=O)NCc1ccccc1C(=O)N[C@H](C)c1cccc2ccccc12] |
| 17 | 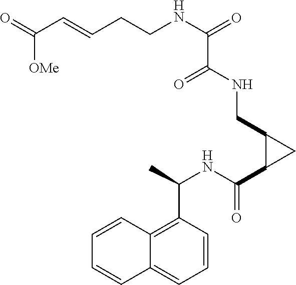 |
| 18 | 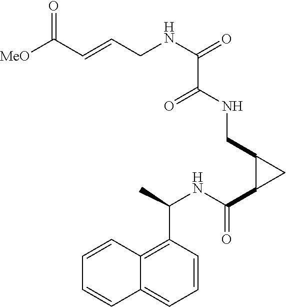 |
| 19 | 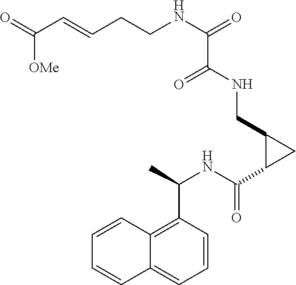 |
| 20 | 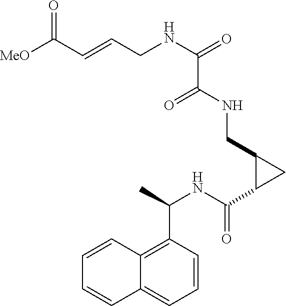 |
| 21 | 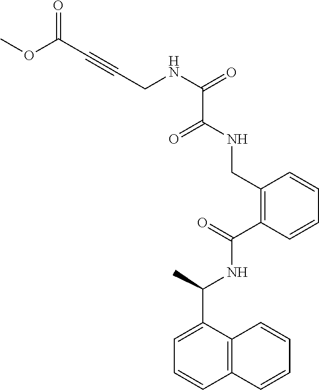 [SMILES: COC(=O)C#CCNC(=O)C(=O)NCc1ccccc1C(=O)N[C@H](C)c1cccc2ccccc12] |
| 22 | 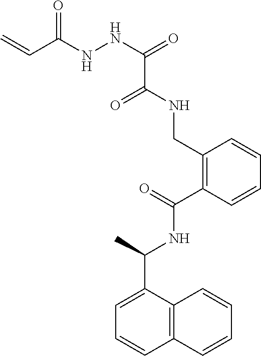 [SMILES: C=CC(=O)NNC(=O)C(=O)NCc1ccccc1C(=O)N[C@H](C)c1cccc2ccccc12] |
| 23 | 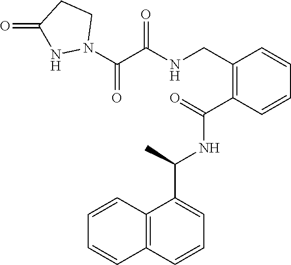 [SMILES: C[C@@H](NC(=O)c1ccccc1CNC(=O)C(=O)N1CCC(=O)N1)c1cccc2ccccc12] |
| 24 | 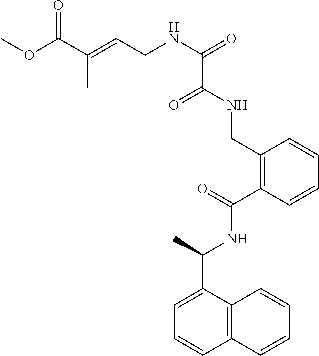 [SMILES: COC(=O)/C(C)=C/CNC(=O)C(=O)NCc1ccccc1C(=O)N[C@H](C)c1cccc2ccccc12] |
| 25 | 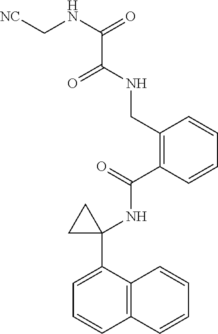 [SMILES: N#CCNC(=O)C(=O)NCc1ccccc1C(=O)NC1(c2cccc3ccccc23)CC1] |
| 26 | 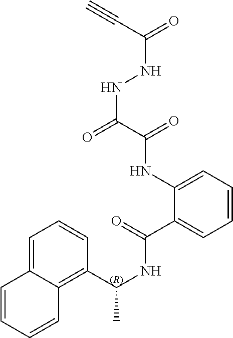 |
| 27 | 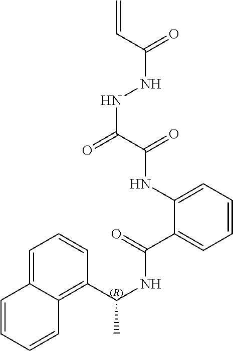 |
| 28 | 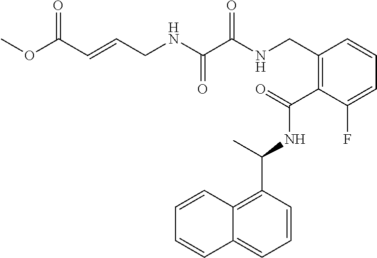 [SMILES: COC(=O)/C=C/CNC(=O)C(=O)NCc1cccc(F)c1C(=O)N[C@H](C)c1cccc2ccccc12] |
| 29 | 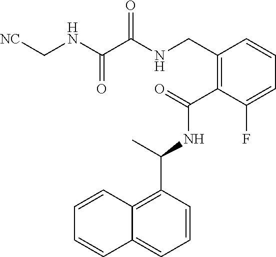 [SMILES: C[C@@H](NC(=O)c1c(F)cccc1CNC(=O)C(=O)NCC#N)c1cccc2ccccc12] |
| 30 | 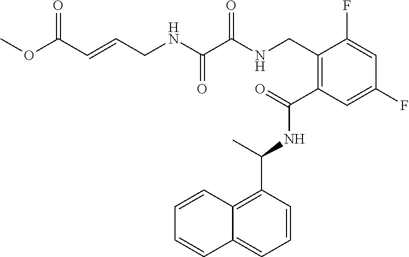 [SMILES: COC(=O)/C=C/CNC(=O)C(=O)NCc1c(F)cc(F)cc1C(=O)N[C@H](C)c1cccc2ccccc12] |
| 31 | 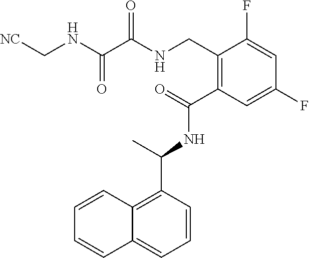 [SMILES: C[C@@H](NC(=O)c1cc(F)cc(F)c1CNC(=O)C(=O)NCC#N)c1cccc2ccccc12] |
| 32 | 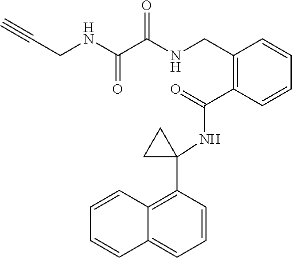 [SMILES: C#CCNC(=O)C(=O)NCc1ccccc1C(=O)NC1(c2cccc3ccccc23)CC1] |
| 33 | 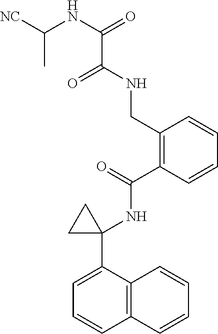 [SMILES: CC(C#N)NC(=O)C(=O)NCc1ccccc1C(=O)NC1(c2cccc3ccccc23)CC1] |
| 34 | 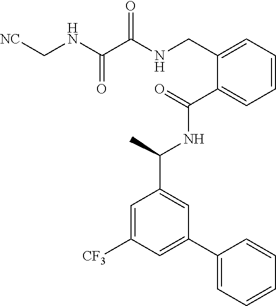 [SMILES: C[C@@H](NC(=O)c1ccccc1CNC(=O)C(=O)NCC#N)c1cc(-c2ccccc2)cc(C(F)(F)F)c1] |
| 35 | 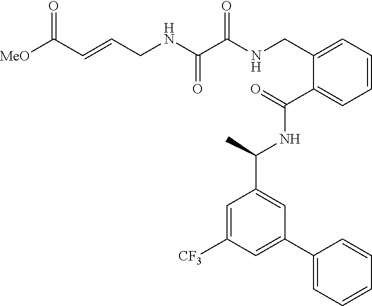 [SMILES: COC(=O)/C=C/CNC(=O)C(=O)NCc1ccccc1C(=O)N[C@H](C)c1cc(-c2ccccc2)cc(C(F)(F)F)c1] |
| 36 | 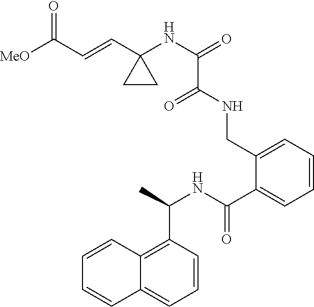 [SMILES: COC(=O)/C=C/C1(NC(=O)C(=O)NCc2ccccc2C(=O)N[C@H](C)c2cccc3ccccc23)CC1] |
| 37 | 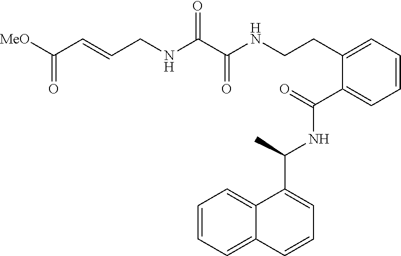 [SMILES: COC(=O)/C=C/CNC(=O)C(=O)NCCc1ccccc1C(=O)N[C@H](C)c1cccc2ccccc12] |
| 38 | 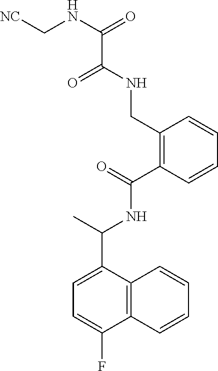 [SMILES: CC(NC(=O)c1ccccc1CNC(=O)C(=O)NCC#N)c1ccc(F)c2ccccc12] |
| 39 | 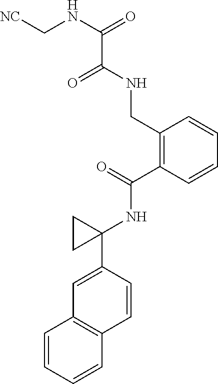 [SMILES: N#CCNC(=O)C(=O)NCc1ccccc1C(=O)NC1(c2ccc3ccccc3c2)CC1] |
| 40 | 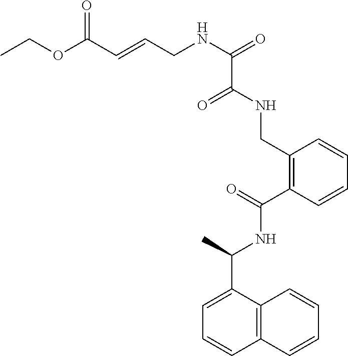 [SMILES: CCOC(=O)/C=C/CNC(=O)C(=O)NCc1ccccc1C(=O)N[C@H](C)c1cccc2ccccc12] |
| 41 | 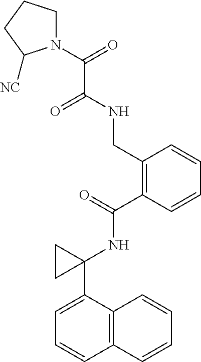 [SMILES: N#CC1CCCN1C(=O)C(=O)NCc1ccccc1C(=O)NC1(c2cccc3ccccc23)CC1] |
| 42 | 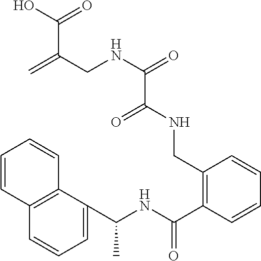 [SMILES: C=C(CNC(=O)C(=O)NCc1ccccc1C(=O)N[C@H](C)c1cccc2ccccc12)C(=O)O] |
| 43 | 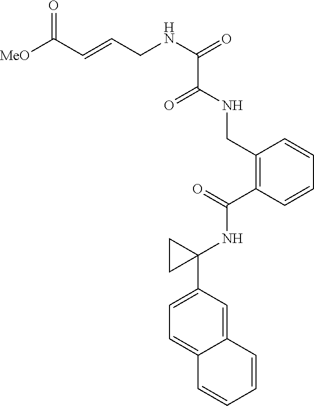 [SMILES: COC(=O)/C=C/CNC(=O)C(=O)NCc1ccccc1C(=O)NC1(c2ccc3ccccc3c2)CC1] |
| 44 | 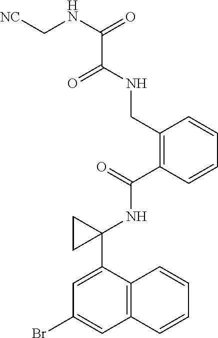 [SMILES: N#CCNC(=O)C(=O)NCc1ccccc1C(=O)NC1(c2cc(Br)cc3ccccc23)CC1] |
| 45 | 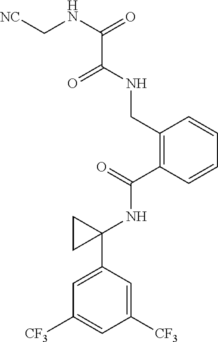 [SMILES: N#CCNC(=O)C(=O)NCc1ccccc1C(=O)NC1(c2cc(C(F)(F)F)cc(C(F)(F)F)c2)CC1] |
| 46 | 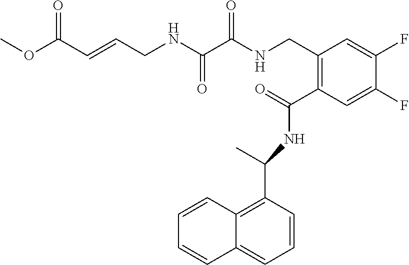 [SMILES: COC(=O)/C=C/CNC(=O)C(=O)NCc1cc(F)c(F)cc1C(=O)N[C@H](C)c1cccc2ccccc12] |
| 47 | 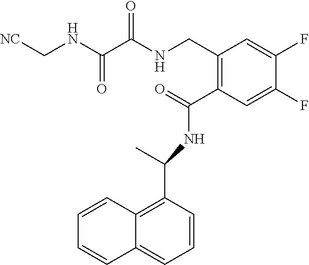 [SMILES: C[C@@H](NC(=O)c1cc(F)c(F)cc1CNC(=O)C(=O)NCC#N)c1cccc2ccccc12] |
| 48 | 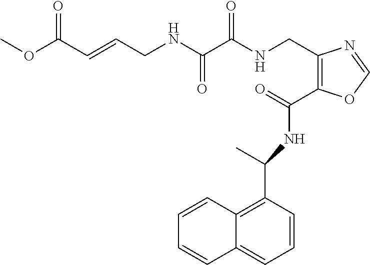 [SMILES: COC(=O)/C=C/CNC(=O)C(=O)NCc1ncoc1C(=O)N[C@H](C)c1cccc2ccccc12] |
| 49 | 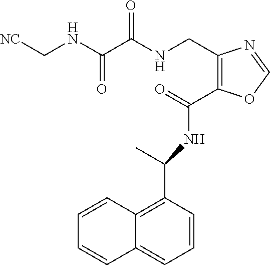 [SMILES: C[C@@H](NC(=O)c1ocnc1CNC(=O)C(=O)NCC#N)c1cccc2ccccc12] |
| 50 | 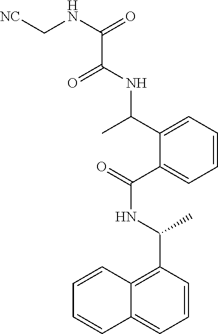 |
| 51 | 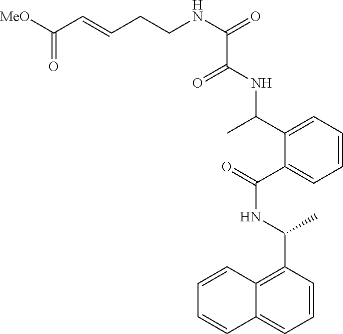 |
| 52 | 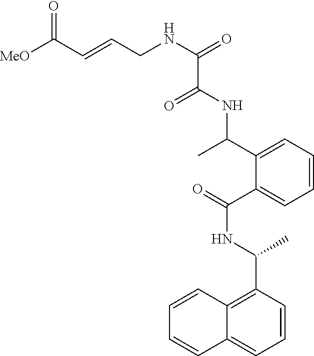 |
| 53 | 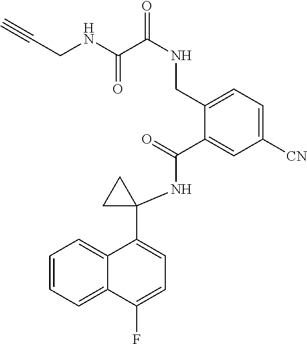 [SMILES: C#CCNC(=O)C(=O)NCc1ccc(C#N)cc1C(=O)NC1(c2ccc(F)c3ccccc23)CC1] |
| 54 | 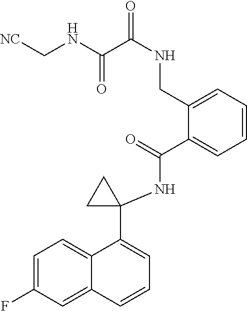 [SMILES: N#CCNC(=O)C(=O)NCc1ccccc1C(=O)NC1(c2cccc3cc(F)ccc23)CC1] |
| 55 | 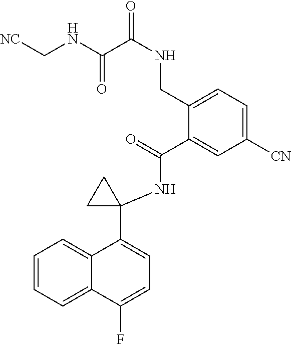 [SMILES: N#CCNC(=O)C(=O)NCc1ccc(C#N)cc1C(=O)NC1(c2ccc(F)c3ccccc23)CC1] |
| 56 | 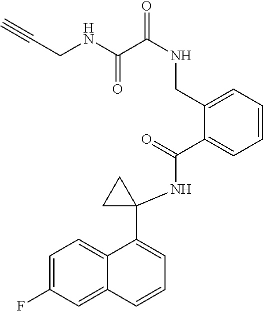 [SMILES: C#CCNC(=O)C(=O)NCc1ccccc1C(=O)NC1(c2cccc3cc(F)ccc23)CC1] |
| 57 | 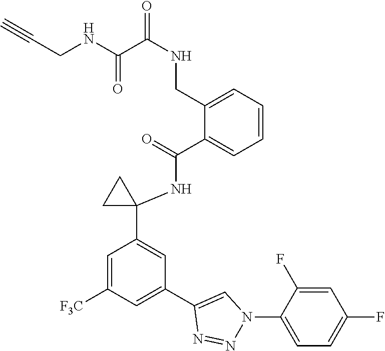 [SMILES: C#CCNC(=O)C(=O)NCc1ccccc1C(=O)NC1(c2cc(-c3cn(-c4ccc(F)cc4F)nn3)cc(C(F)(F)F)c2)CC1] |
| 58 | 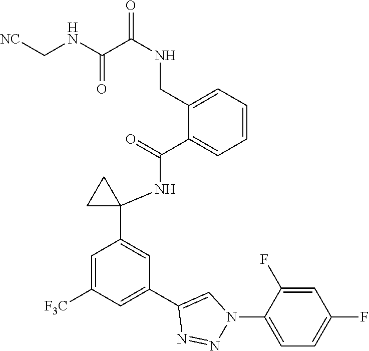 [SMILES: N#CCNC(=O)C(=O)NCc1ccccc1C(=O)NC1(c2cc(-c3cn(-c4ccc(F)cc4F)nn3)cc(C(F)(F)F)c2)CC1] |
| 59 | 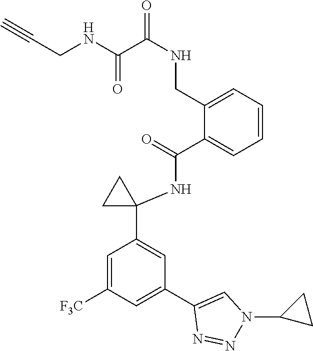 [SMILES: C#CCNC(=O)C(=O)NCc1ccccc1C(=O)NC1(c2cc(-c3cn(C4CC4)nn3)cc(C(F)(F)F)c2)CC1] |
| 60 | 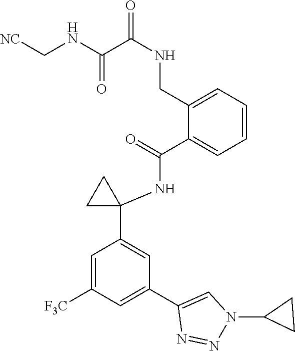 [SMILES: N#CCNC(=O)C(=O)NCc1ccccc1C(=O)NC1(c2cc(-c3cn(C4CC4)nn3)cc(C(F)(F)F)c2)CC1] |
| 61 | 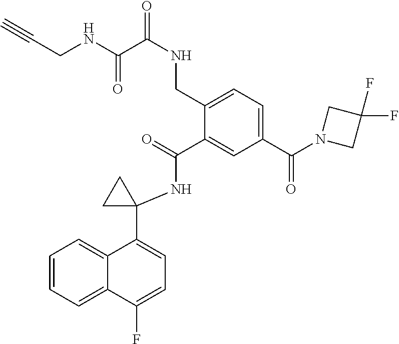 [SMILES: C#CCNC(=O)C(=O)NCc1ccc(C(=O)N2CC(F)(F)C2)cc1C(=O)NC1(c2ccc(F)c3ccccc23)CC1] |
| 62 | 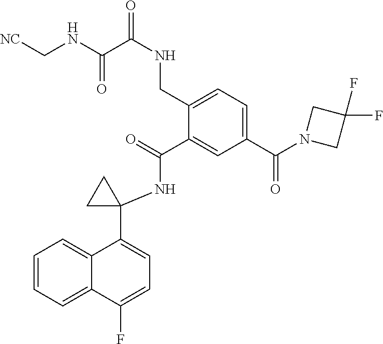 [SMILES: N#CCNC(=O)C(=O)NCc1ccc(C(=O)N2CC(F)(F)C2)cc1C(=O)NC1(c2ccc(F)c3ccccc23)CC1] |
| 63 | 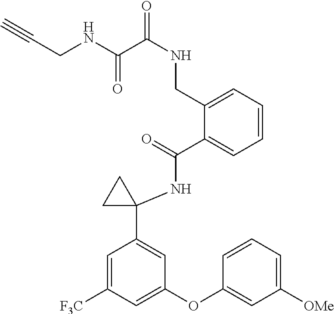 [SMILES: C#CCNC(=O)C(=O)NCc1ccccc1C(=O)NC1(c2cc(Oc3cccc(OC)c3)cc(C(F)(F)F)c2)CC1] |
| 64 | 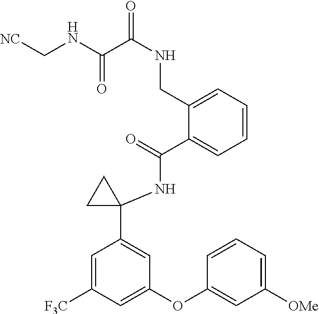 [SMILES: COc1cccc(Oc2cc(C(F)(F)F)cc(C3(NC(=O)c4ccccc4CNC(=O)C(=O)NCC#N)CC3)c2)c1] |
| 65 | 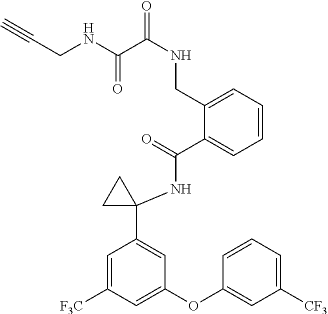 [SMILES: C#CCNC(=O)C(=O)NCc1ccccc1C(=O)NC1(c2cc(Oc3cccc(C(F)(F)F)c3)cc(C(F)(F)F)c2)CC1] |
| 66 | 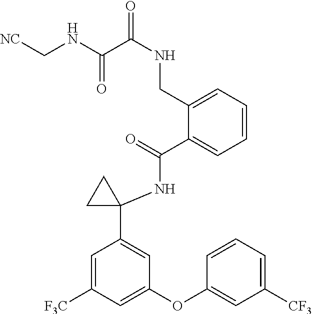 [SMILES: N#CCNC(=O)C(=O)NCc1ccccc1C(=O)NC1(c2cc(Oc3cccc(C(F)(F)F)c3)cc(C(F)(F)F)c2)CC1] |
| 67 | 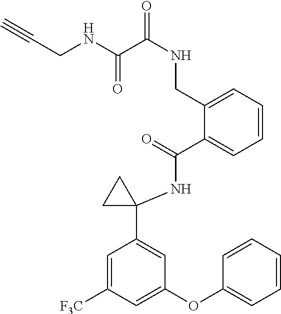 [SMILES: C#CCNC(=O)C(=O)NCc1ccccc1C(=O)NC1(c2cc(Oc3ccccc3)cc(C(F)(F)F)c2)CC1] |
| 68 | 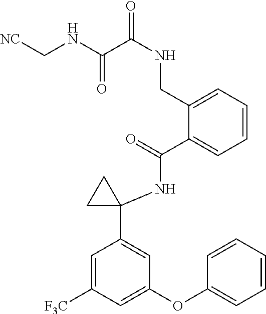 [SMILES: N#CCNC(=O)C(=O)NCc1ccccc1C(=O)NC1(c2cc(Oc3ccccc3)cc(C(F)(F)F)c2)CC1] |
| 69 | 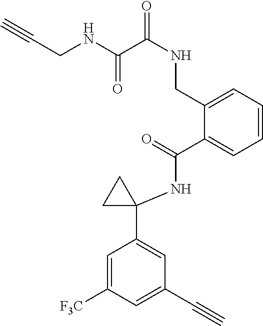 [SMILES: C#CCNC(=O)C(=O)NCc1ccccc1C(=O)NC1(c2cc(C#C)cc(C(F)(F)F)c2)CC1] |
| 70 | 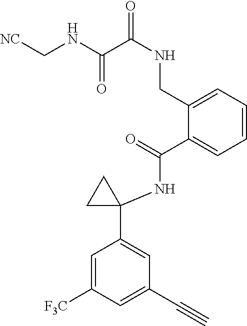 [SMILES: C#Cc1cc(C(F)(F)F)cc(C2(NC(=O)c3ccccc3CNC(=O)C(=O)NCC#N)CC2)c1] |
| 71 | 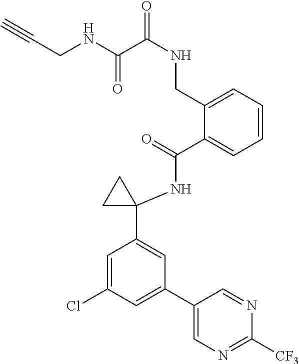 [SMILES: C#CCNC(=O)C(=O)NCc1ccccc1C(=O)NC1(c2cc(Cl)cc(-c3cnc(C(F)(F)F)nc3)c2)CC1] |
| 72 | 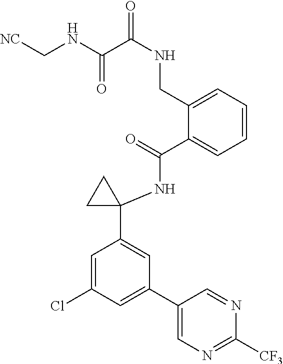 [SMILES: N#CCNC(=O)C(=O)NCc1ccccc1C(=O)NC1(c2cc(Cl)cc(-c3cnc(C(F)(F)F)nc3)c2)CC1] |
| 73 | 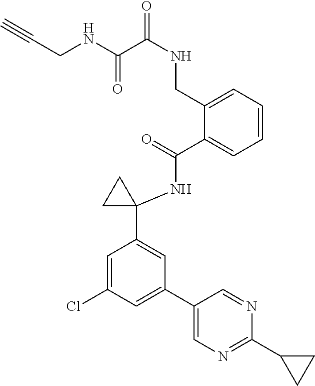 [SMILES: C#CCNC(=O)C(=O)NCc1ccccc1C(=O)NC1(c2cc(Cl)cc(-c3cnc(C4CC4)nc3)c2)CC1] |
| 74 | 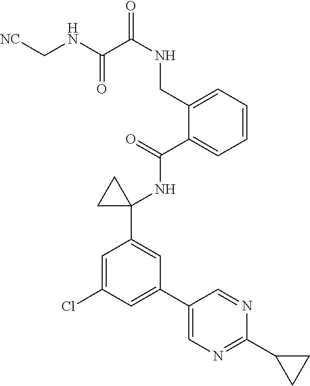 [SMILES: N#CCNC(=O)C(=O)NCc1ccccc1C(=O)NC1(c2cc(Cl)cc(-c3cnc(C4CC4)nc3)c2)CC1] |
| 75 | 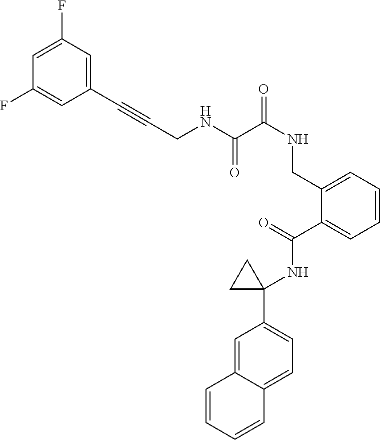 [SMILES: O=C(NCC#Cc1cc(F)cc(F)c1)C(=O)NCc1ccccc1C(=O)NC1(c2ccc3ccccc3c2)CC1] |
| 76 | 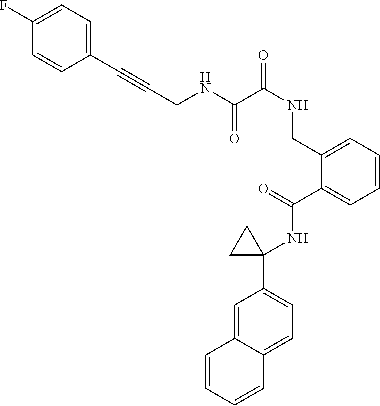 [SMILES: O=C(NCC#Cc1ccc(F)cc1)C(=O)NCc1ccccc1C(=O)NC1(c2ccc3ccccc3c2)CC1] |
| 77 | 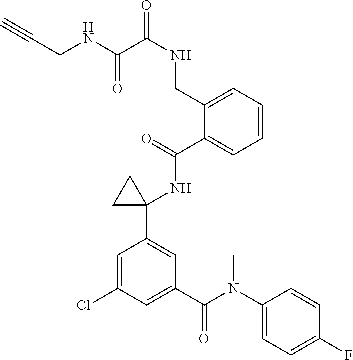 [SMILES: C#CCNC(=O)C(=O)NCc1ccccc1C(=O)NC1(c2cc(Cl)cc(C(=O)N(C)c3ccc(F)cc3)c2)CC1] |
| 78 | 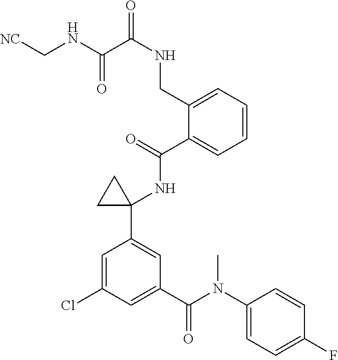 [SMILES: CN(C(=O)c1cc(Cl)cc(C2(NC(=O)c3ccccc3CNC(=O)C(=O)NCC#N)CC2)c1)c1ccc(F)cc1] |
| 79 | 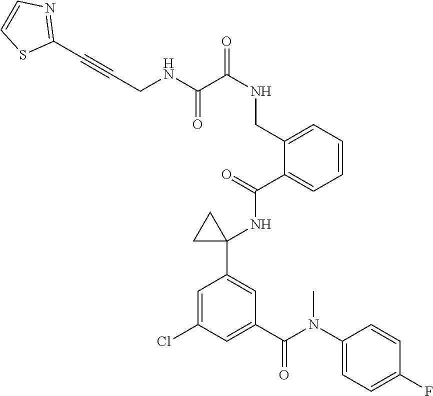 [SMILES: CN(C(=O)c1cc(Cl)cc(C2(NC(=O)c3ccccc3CNC(=O)C(=O)NCC#Cc3nccs3)CC2)c1)c1ccc(F)cc1] |
| 80 | 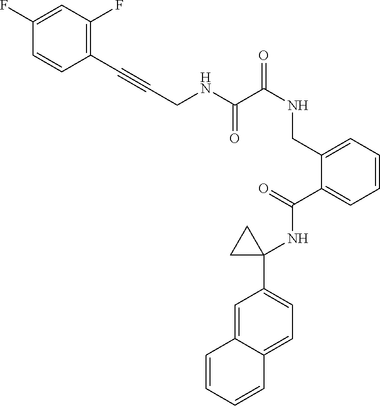 [SMILES: O=C(NCC#Cc1ccc(F)cc1F)C(=O)NCc1ccccc1C(=O)NC1(c2ccc3ccccc3c2)CC1] |
| 81 | 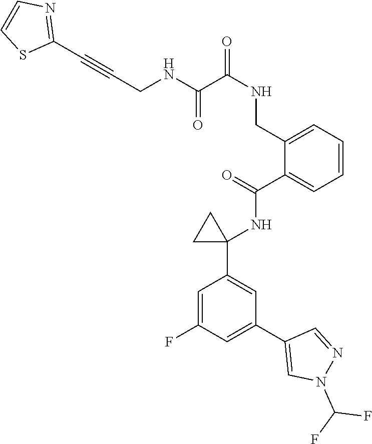 [SMILES: O=C(NCC#Cc1nccs1)C(=O)NCc1ccccc1C(=O)NC1(c2cc(F)cc(-c3cnn(C(F)F)c3)c2)CC1] |
| 82 | 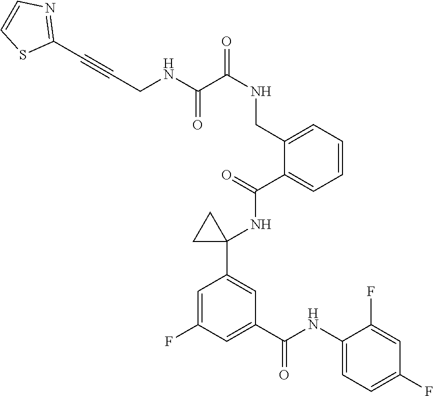 [SMILES: O=C(NCC#Cc1nccs1)C(=O)NCc1ccccc1C(=O)NC1(c2cc(F)cc(C(=O)Nc3ccc(F)cc3F)c2)CC1] |
| 83 | 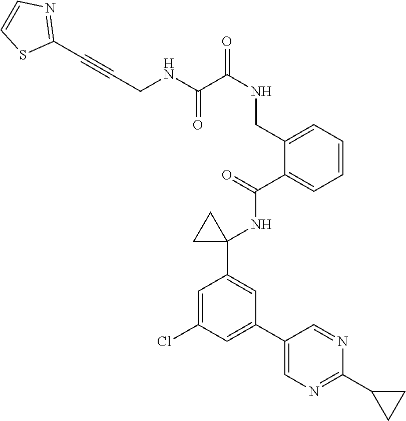 [SMILES: O=C(NCC#Cc1nccs1)C(=O)NCc1ccccc1C(=O)NC1(c2cc(Cl)cc(-c3cnc(C4CC4)nc3)c2)CC1] |
| 84 | 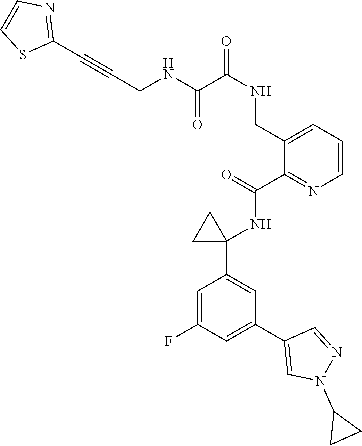 [SMILES: O=C(NCC#Cc1nccs1)C(=O)NCc1cccnc1C(=O)NC1(c2cc(F)cc(-c3cnn(C4CC4)c3)c2)CC1] |
| 85 | 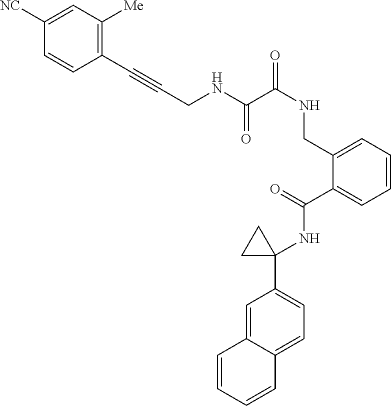 [SMILES: Cc1cc(C#N)ccc1C#CCNC(=O)C(=O)NCc1ccccc1C(=O)NC1(c2ccc3ccccc3c2)CC1] |
| 86 | 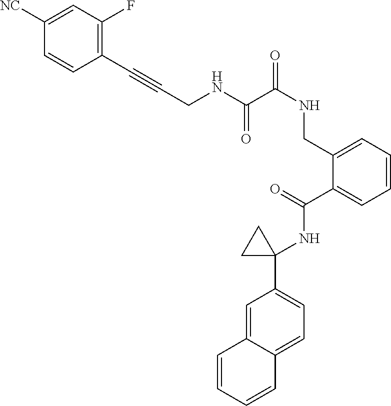 [SMILES: N#Cc1ccc(C#CCNC(=O)C(=O)NCc2ccccc2C(=O)NC2(c3ccc4ccccc4c3)CC2)c(F)c1] |
| 87 | 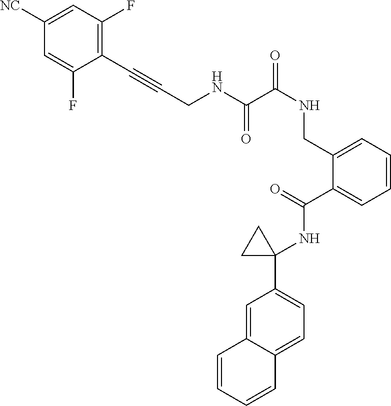 [SMILES: N#Cc1cc(F)c(C#CCNC(=O)C(=O)NCc2ccccc2C(=O)NC2(c3ccc4ccccc4c3)CC2)c(F)c1] |
| 88 | 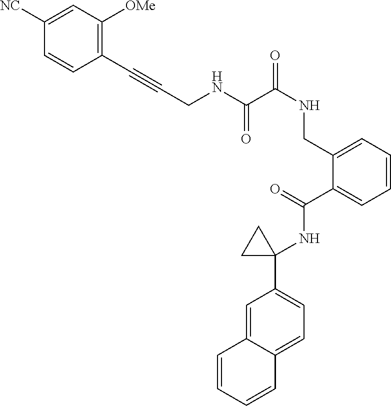 [SMILES: COc1cc(C#N)ccc1C#CCNC(=O)C(=O)NCc1ccccc1C(=O)NC1(c2ccc3ccccc3c2)CC1] |
| 89 | 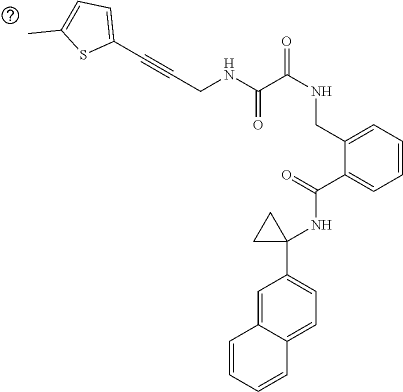 [SMILES: Cc1ccc(C#CCNC(=O)C(=O)NCc2ccccc2C(=O)NC2(c3ccc4ccccc4c3)CC2)s1] |
| 90 | 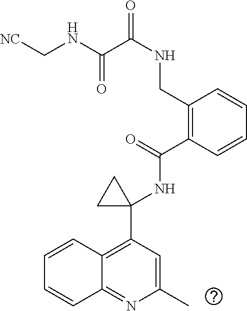 [SMILES: Cc1cc(C2(NC(=O)c3ccccc3CNC(=O)C(=O)NCC#N)CC2)c2ccccc2n1] |
| 91 | 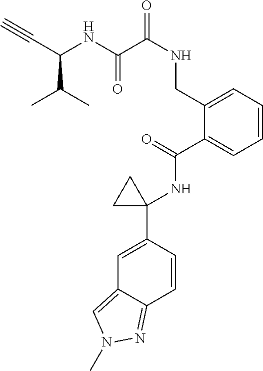 [SMILES: C#C[C@@H](NC(=O)C(=O)NCc1ccccc1C(=O)NC1(c2ccc3nn(C)cc3c2)CC1)C(C)C] |
| 92 | 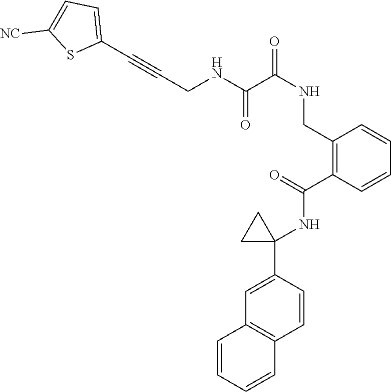 [SMILES: N#Cc1ccc(C#CCNC(=O)C(=O)NCc2ccccc2C(=O)NC2(c3ccc4ccccc4c3)CC2)s1] |
| 93 | 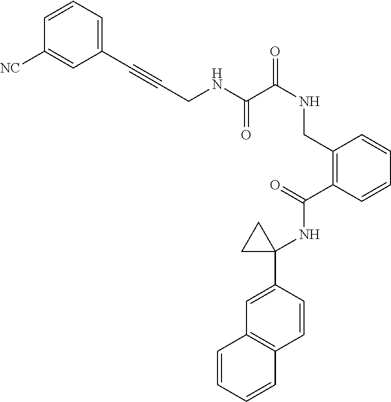 [SMILES: N#Cc1cccc(C#CCNC(=O)C(=O)NCc2ccccc2C(=O)NC2(c3ccc4ccccc4c3)CC2)c1] |
| 94 | 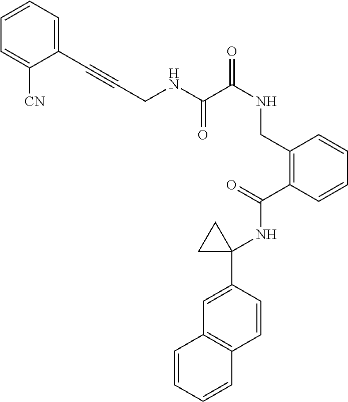 [SMILES: N#Cc1ccccc1C#CCNC(=O)C(=O)NCc1ccccc1C(=O)NC1(c2ccc3ccccc3c2)CC1] |
| 95 | 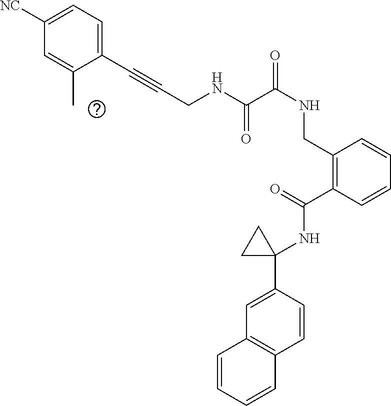 [SMILES: Cc1cc(C#N)ccc1C#CCNC(=O)C(=O)NCc1ccccc1C(=O)NC1(c2ccc3ccccc3c2)CC1] |
| 96 | 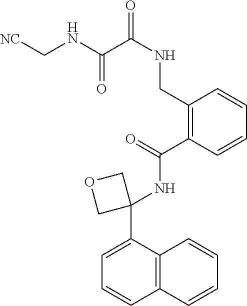 [SMILES: N#CCNC(=O)C(=O)NCc1ccccc1C(=O)NC1(c2cccc3ccccc23)COC1] |
| 97 | 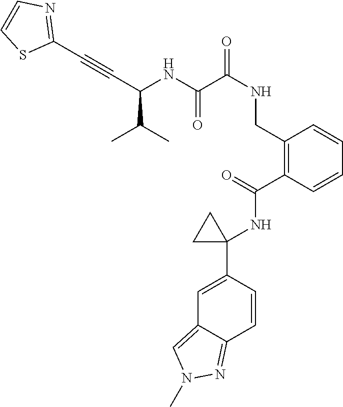 [SMILES: CC(C)[C@@H](C#Cc1nccs1)NC(=O)C(=O)NCc1ccccc1C(=O)NC1(c2ccc3nn(C)cc3c2)CC1] |
| 98 | 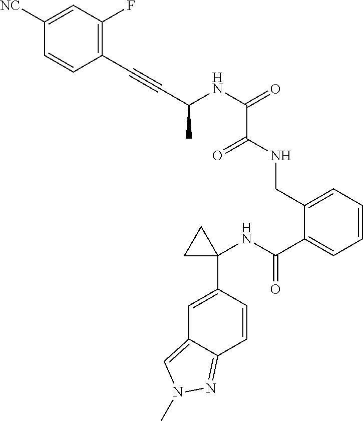 [SMILES: C[C@@H](C#Cc1ccc(C#N)cc1F)NC(=O)C(=O)NCc1ccccc1C(=O)NC1(c2ccc3nn(C)cc3c2)CC1] |
| 99 | 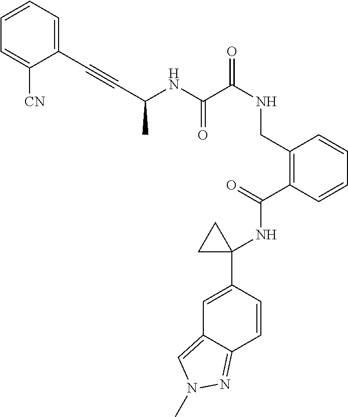 [SMILES: C[C@@H](C#Cc1ccccc1C#N)NC(=O)C(=O)NCc1ccccc1C(=O)NC1(c2ccc3nn(C)cc3c2)CC1] |
| 100 | 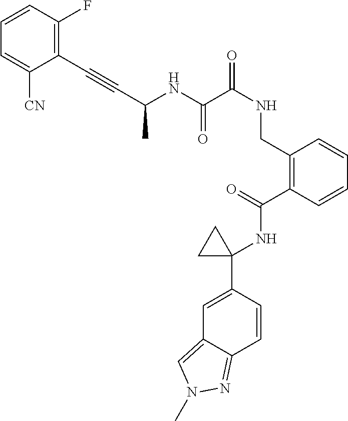 [SMILES: C[C@@H](C#Cc1c(F)cccc1C#N)NC(=O)C(=O)NCc1ccccc1C(=O)NC1(c2ccc3nn(C)cc3c2)CC1] |
| 101 | 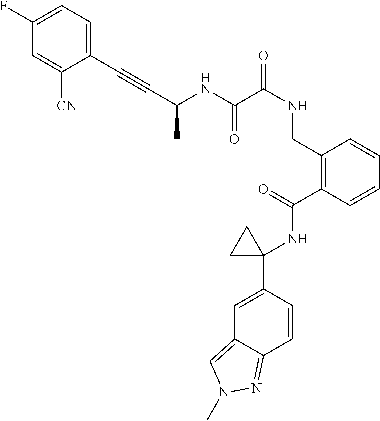 [SMILES: C[C@@H](C#Cc1ccc(F)cc1C#N)NC(=O)C(=O)NCc1ccccc1C(=O)NC1(c2ccc3nn(C)cc3c2)CC1] |
| 102 | 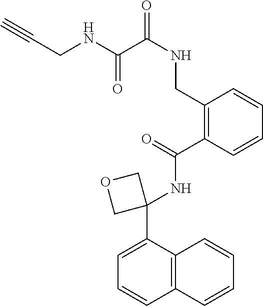 [SMILES: C#CCNC(=O)C(=O)NCc1ccccc1C(=O)NC1(c2cccc3ccccc23)COC1] |
| 103 | 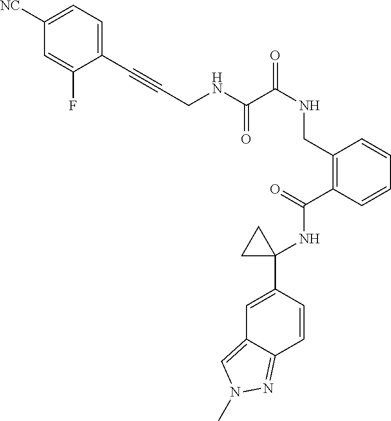 [SMILES: Cn1cc2cc(C3(NC(=O)c4ccccc4CNC(=O)C(=O)NCC#Cc4ccc(C#N)cc4F)CC3)ccc2n1] |
| 104 | 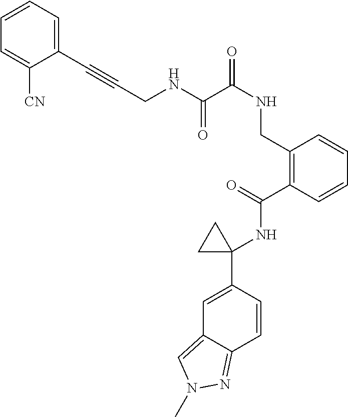 [SMILES: Cn1cc2cc(C3(NC(=O)c4ccccc4CNC(=O)C(=O)NCC#Cc4ccccc4C#N)CC3)ccc2n1] |
| 105 | 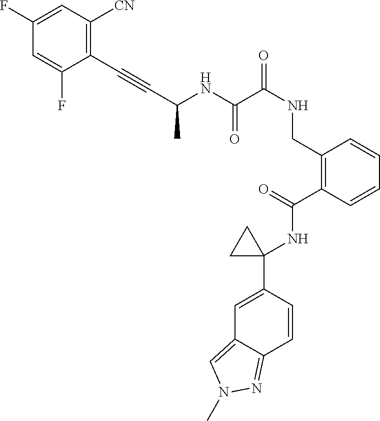 [SMILES: C[C@@H](C#Cc1c(F)cc(F)cc1C#N)NC(=O)C(=O)NCc1ccccc1C(=O)NC1(c2ccc3nn(C)cc3c2)CC1] |
| 106 | 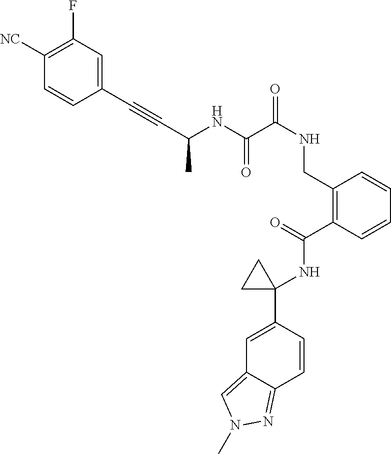 [SMILES: C[C@@H](C#Cc1ccc(C#N)c(F)c1)NC(=O)C(=O)NCc1ccccc1C(=O)NC1(c2ccc3nn(C)cc3c2)CC1] |
| 107 | 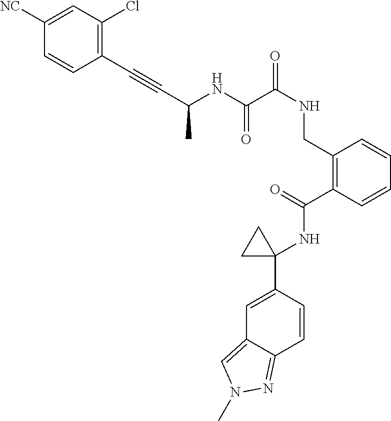 [SMILES: C[C@@H](C#Cc1ccc(C#N)cc1Cl)NC(=O)C(=O)NCc1ccccc1C(=O)NC1(c2ccc3nn(C)cc3c2)CC1] |
| 108 | 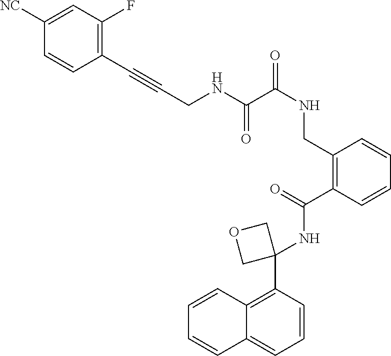 [SMILES: N#Cc1ccc(C#CCNC(=O)C(=O)NCc2ccccc2C(=O)NC2(c3cccc4ccccc34)COC2)c(F)c1] |
| 109 | 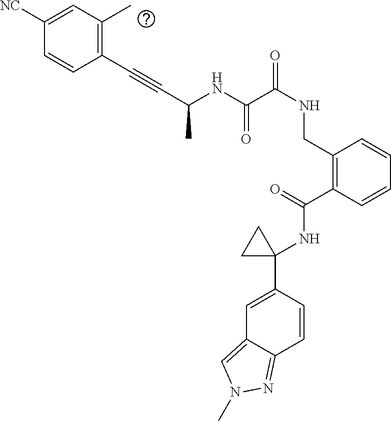 [SMILES: Cc1cc(C#N)ccc1C#C[C@H](C)NC(=O)C(=O)NCc1ccccc1C(=O)NC1(c2ccc3nn(C)cc3c2)CC1] |
| 110 | 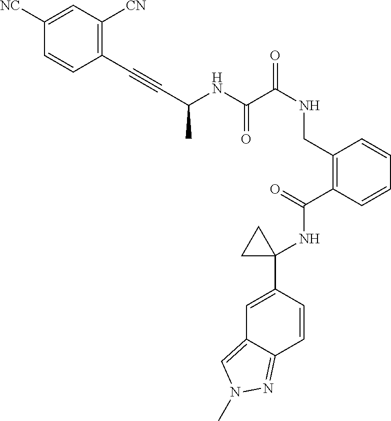 [SMILES: C[C@@H](C#Cc1ccc(C#N)cc1C#N)NC(=O)C(=O)NCc1ccccc1C(=O)NC1(c2ccc3nn(C)cc3c2)CC1] |
| 111 | 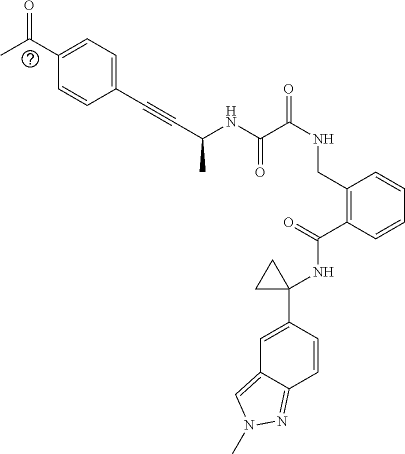 [SMILES: CC(=O)c1ccc(C#C[C@H](C)NC(=O)C(=O)NCc2ccccc2C(=O)NC2(c3ccc4nn(C)cc4c3)CC2)cc1] |
| 112 | 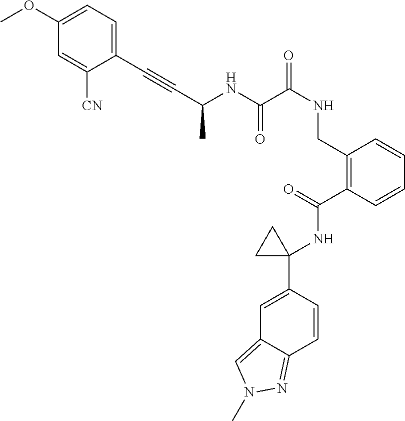 [SMILES: COc1ccc(C#C[C@H](C)NC(=O)C(=O)NCc2ccccc2C(=O)NC2(c3ccc4nn(C)cc4c3)CC2)c(C#N)c1] |
| 113 | 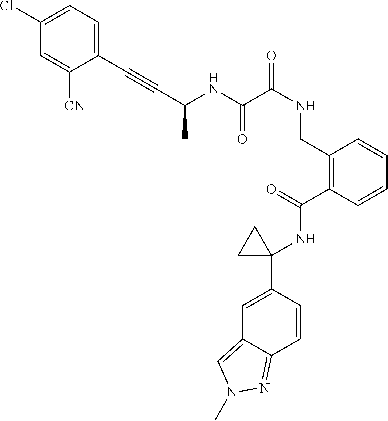 [SMILES: C[C@@H](C#Cc1ccc(Cl)cc1C#N)NC(=O)C(=O)NCc1ccccc1C(=O)NC1(c2ccc3nn(C)cc3c2)CC1] |
| 114 | 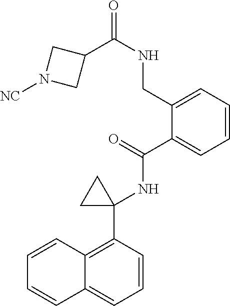 [SMILES: N#CN1CC(C(=O)NCc2ccccc2C(=O)NC2(c3cccc4ccccc34)CC2)C1] |
| 115 | 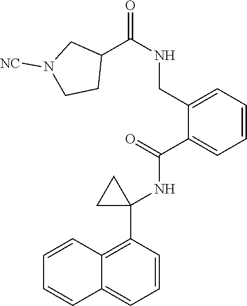 [SMILES: N#CN1CCC(C(=O)NCc2ccccc2C(=O)NC2(c3cccc4ccccc34)CC2)C1] |
| 116 | 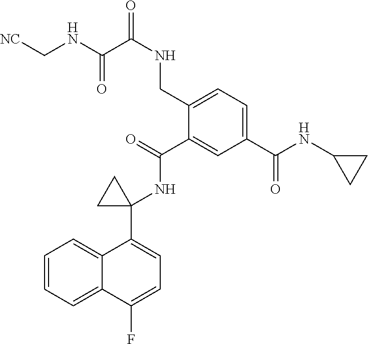 [SMILES: N#CCNC(=O)C(=O)NCc1ccc(C(=O)NC2CC2)cc1C(=O)NC1(c2ccc(F)c3ccccc23)CC1] |
| 117 | 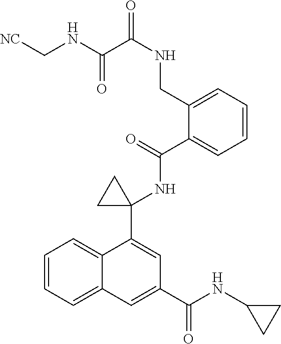 [SMILES: N#CCNC(=O)C(=O)NCc1ccccc1C(=O)NC1(c2cc(C(=O)NC3CC3)cc3ccccc23)CC1] |
| 118 | 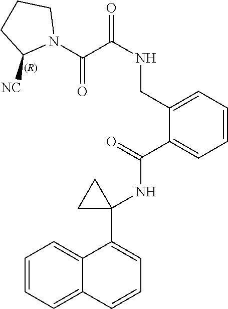 |
| 119 | 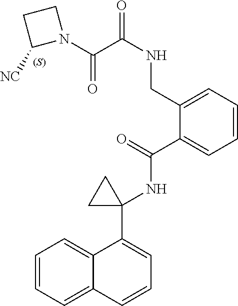 |
| 120 | 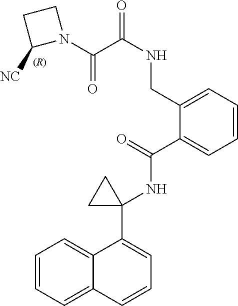 |
| 121 | 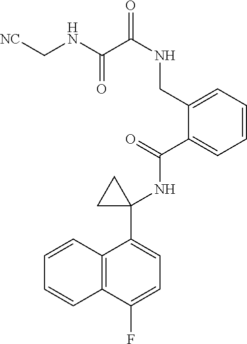 [SMILES: N#CCNC(=O)C(=O)NCc1ccccc1C(=O)NC1(c2ccc(F)c3ccccc23)CC1] |
| 122 | 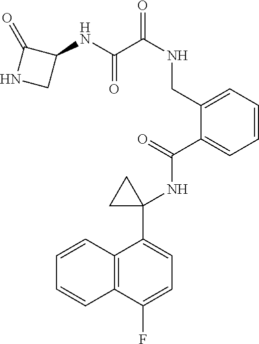 [SMILES: O=C(NCc1ccccc1C(=O)NC1(c2ccc(F)c3ccccc23)CC1)C(=O)N[C@H]1CNC1=O] |
| 123 | 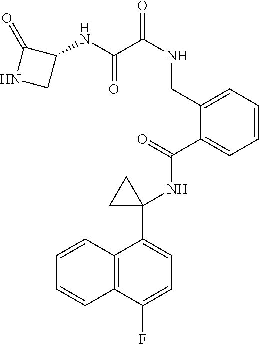 [SMILES: O=C(NCc1ccccc1C(=O)NC1(c2ccc(F)c3ccccc23)CC1)C(=O)N[C@@H]1CNC1=O] |
| 124 | 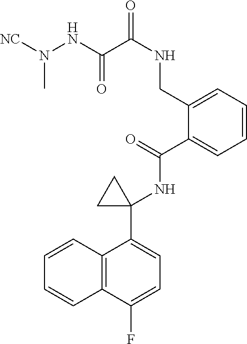 [SMILES: CN(C#N)NC(=O)C(=O)NCc1ccccc1C(=O)NC1(c2ccc(F)c3ccccc23)CC1] |
| 125 | 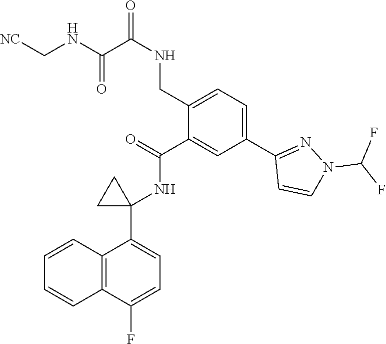 [SMILES: N#CCNC(=O)C(=O)NCc1ccc(-c2ccn(C(F)F)n2)cc1C(=O)NC1(c2ccc(F)c3ccccc23)CC1] |
| 126 | 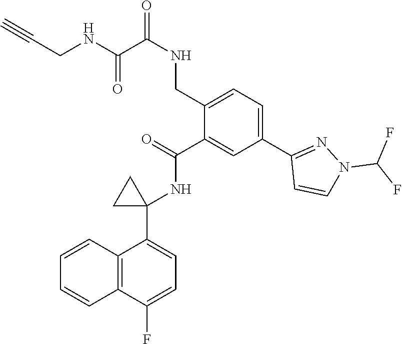 [SMILES: C#CCNC(=O)C(=O)NCc1ccc(-c2ccn(C(F)F)n2)cc1C(=O)NC1(c2ccc(F)c3ccccc23)CC1] |
| 127 | 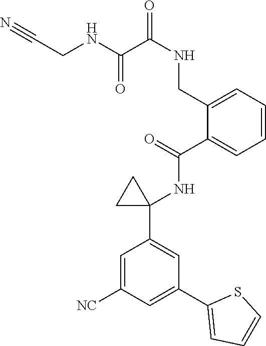 [SMILES: N#CCNC(=O)C(=O)NCc1ccccc1C(=O)NC1(c2cc(C#N)cc(-c3cccs3)c2)CC1] |
| 128 | 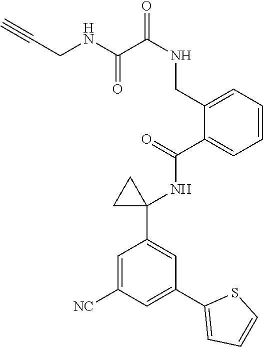 [SMILES: C#CCNC(=O)C(=O)NCc1ccccc1C(=O)NC1(c2cc(C#N)cc(-c3cccs3)c2)CC1] |
| 129 | 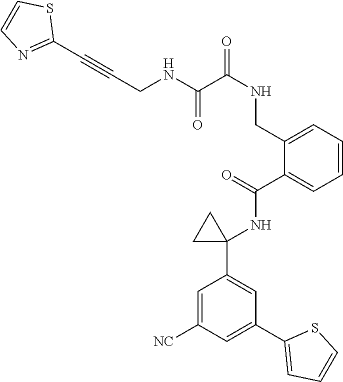 [SMILES: N#Cc1cc(-c2cccs2)cc(C2(NC(=O)c3ccccc3CNC(=O)C(=O)NCC#Cc3nccs3)CC2)c1] |
| 130 | 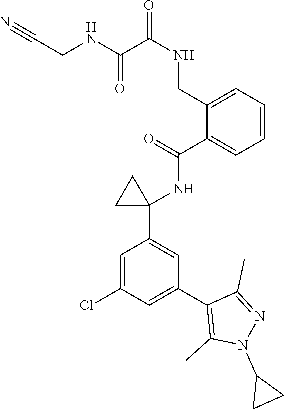 [SMILES: Cc1nn(C2CC2)c(C)c1-c1cc(Cl)cc(C2(NC(=O)c3ccccc3CNC(=O)C(=O)NCC#N)CC2)c1] |
| 131 | 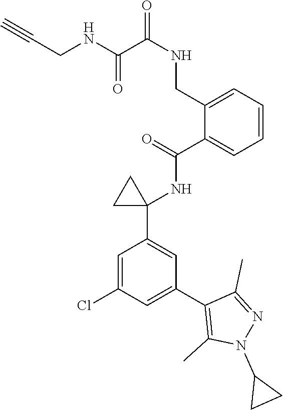 [SMILES: C#CCNC(=O)C(=O)NCc1ccccc1C(=O)NC1(c2cc(Cl)cc(-c3c(C)nn(C4CC4)c3C)c2)CC1] |
| 132 | 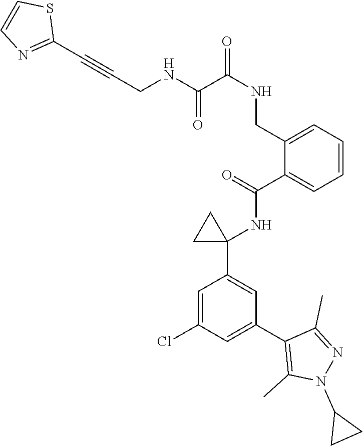 [SMILES: Cc1nn(C2CC2)c(C)c1-c1cc(Cl)cc(C2(NC(=O)c3ccccc3CNC(=O)C(=O)NCC#Cc3nccs3)CC2)c1] |
| 133 | 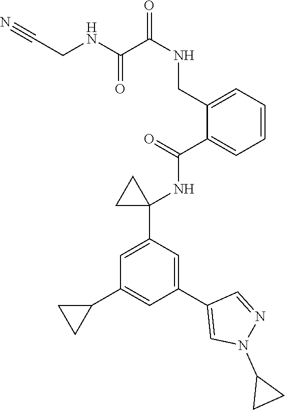 [SMILES: N#CCNC(=O)C(=O)NCc1ccccc1C(=O)NC1(c2cc(-c3cnn(C4CC4)c3)cc(C3CC3)c2)CC1] |
| 134 | 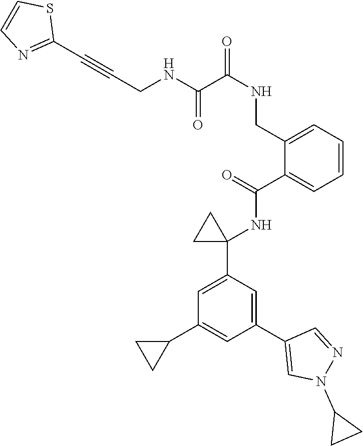 [SMILES: O=C(NCC#Cc1nccs1)C(=O)NCc1ccccc1C(=O)NC1(c2cc(-c3cnn(C4CC4)c3)cc(C3CC3)c2)CC1] |
| 135 | 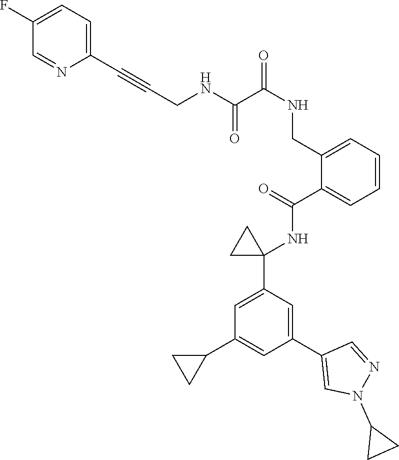 [SMILES: O=C(NCC#Cc1ccc(F)cn1)C(=O)NCc1ccccc1C(=O)NC1(c2cc(-c3cnn(C4CC4)c3)cc(C3CC3)c2)CC1] |
| 136 | 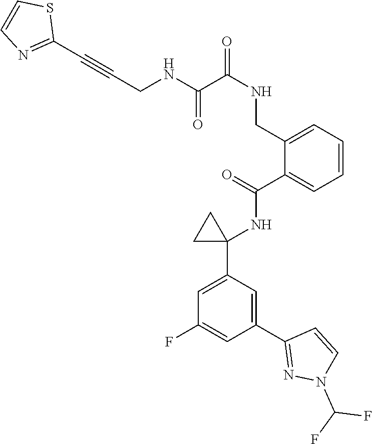 [SMILES: O=C(NCC#Cc1nccs1)C(=O)NCc1ccccc1C(=O)NC1(c2cc(F)cc(-c3ccn(C(F)F)n3)c2)CC1] |
| 137 | 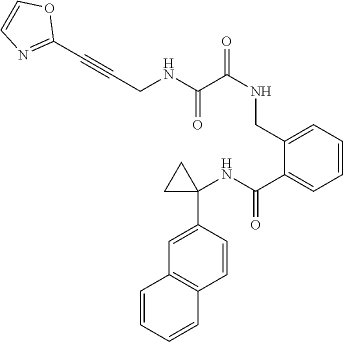 [SMILES: O=C(NCC#Cc1ncco1)C(=O)NCc1ccccc1C(=O)NC1(c2ccc3ccccc3c2)CC1] |
| 138 | 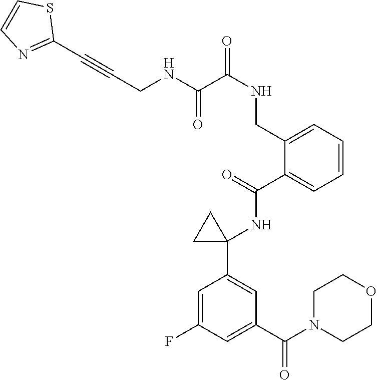 [SMILES: O=C(NCC#Cc1nccs1)C(=O)NCc1ccccc1C(=O)NC1(c2cc(F)cc(C(=O)N3CCOCC3)c2)CC1] |
| 139 | 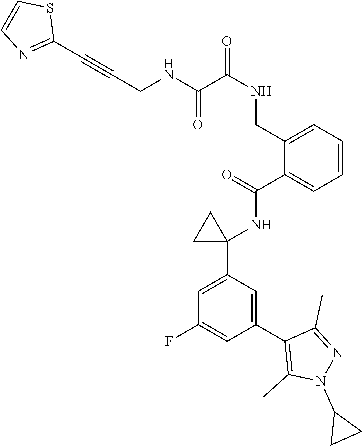 [SMILES: Cc1nn(C2CC2)c(C)c1-c1cc(F)cc(C2(NC(=O)c3ccccc3CNC(=O)C(=O)NCC#Cc3nccs3)CC2)c1] |
| 140 | 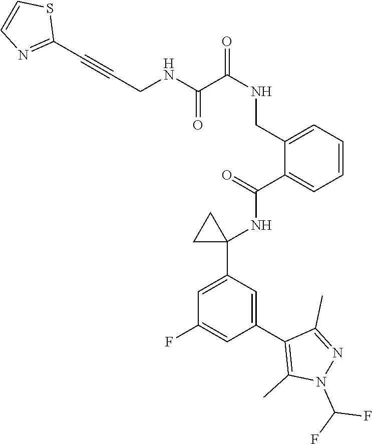 [SMILES: Cc1nn(C(F)F)c(C)c1-c1cc(F)cc(C2(NC(=O)c3ccccc3CNC(=O)C(=O)NCC#Cc3nccs3)CC2)c1] |
| 141 | 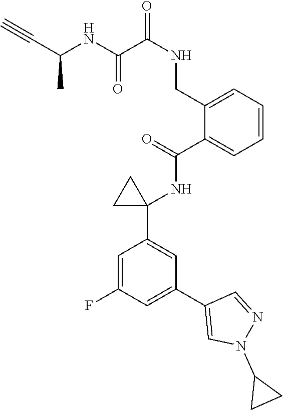 [SMILES: C#C[C@H](C)NC(=O)C(=O)NCc1ccccc1C(=O)NC1(c2cc(F)cc(-c3cnn(C4CC4)c3)c2)CC1] |
| 142 | 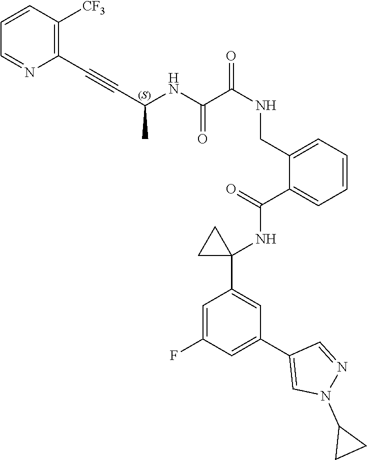 |
| 143 | 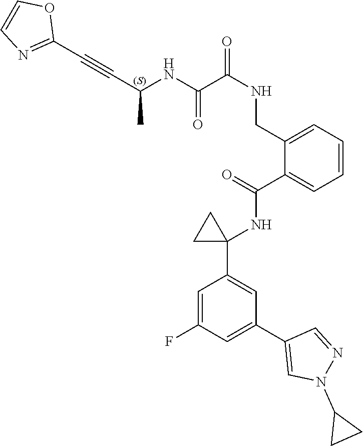 |
| 144 | 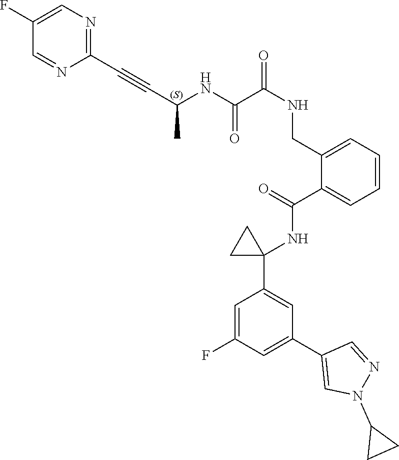 |
| 145 | 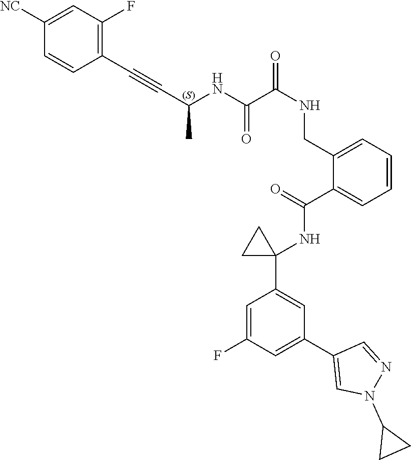 |
| 146 | 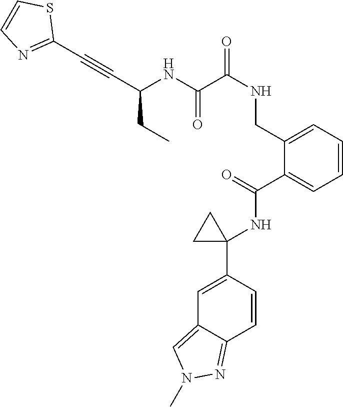 [SMILES: CC[C@@H](C#Cc1nccs1)NC(=O)C(=O)NCc1ccccc1C(=O)NC1(c2ccc3nn(C)cc3c2)CC1] |
| 147 | 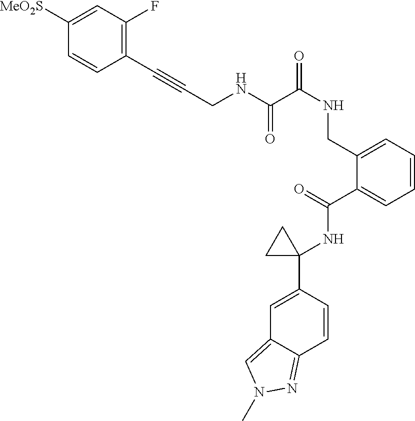 [SMILES: Cn1cc2cc(C3(NC(=O)c4ccccc4CNC(=O)C(=O)NCC#Cc4ccc(S(C)(=O)=O)cc4F)CC3)ccc2n1] |
| 148 | 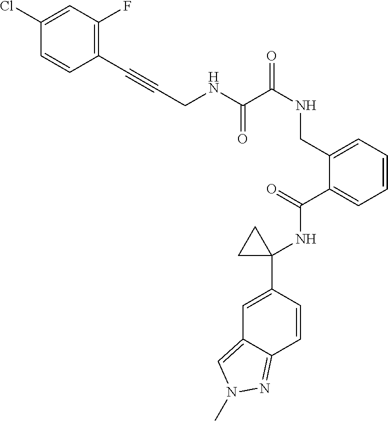 [SMILES: Cn1cc2cc(C3(NC(=O)c4ccccc4CNC(=O)C(=O)NCC#Cc4ccc(Cl)cc4F)CC3)ccc2n1] |
| 149 | 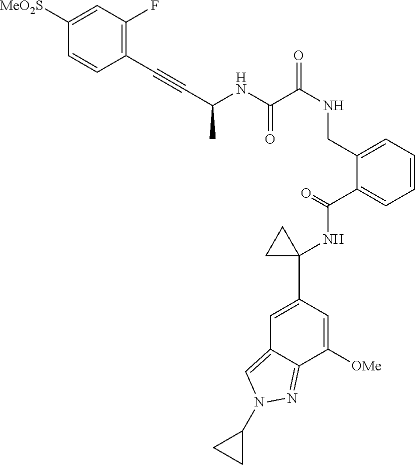 [SMILES: COc1cc(C2(NC(=O)c3ccccc3CNC(=O)C(=O)N[C@@H](C)C#Cc3ccc(S(C)(=O)=O)cc3F)CC2)cc2cn(C3CC3)nc12] |
| 150 | 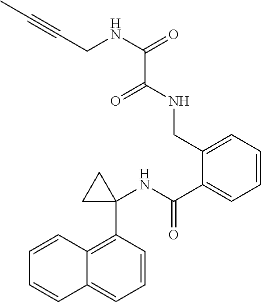 [SMILES: CC#CCNC(=O)C(=O)NCc1ccccc1C(=O)NC1(c2cccc3ccccc23)CC1] |
| 151 | 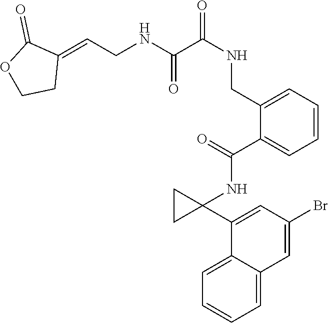 [SMILES: O=C(NC/C=C1\CCOC1=O)C(=O)NCc1ccccc1C(=O)NC1(c2cc(Br)cc3ccccc23)CC1] |
| 152 | 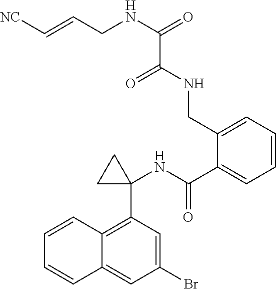 [SMILES: N#C/C=C/CNC(=O)C(=O)NCc1ccccc1C(=O)NC1(c2cc(Br)cc3ccccc23)CC1] |
| 153 | 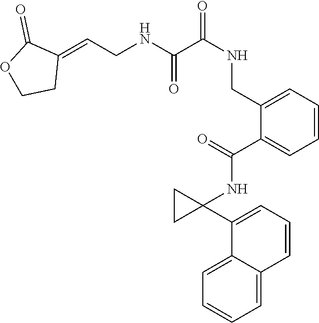 [SMILES: O=C(NC/C=C1\CCOC1=O)C(=O)NCc1ccccc1C(=O)NC1(c2cccc3ccccc23)CC1] |
| 154 | 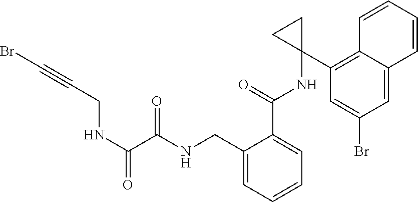 [SMILES: O=C(NCC#CBr)C(=O)NCc1ccccc1C(=O)NC1(c2cc(Br)cc3ccccc23)CC1] |
| 155 | 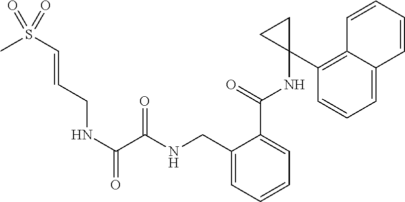 [SMILES: CS(=O)(=O)/C=C/CNC(=O)C(=O)NCc1ccccc1C(=O)NC1(c2cccc3ccccc23)CC1] |
| 156 | 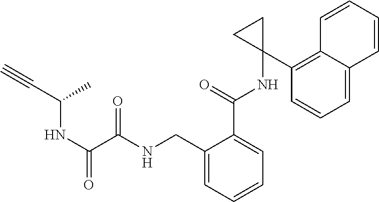 [SMILES: C#C[C@H](C)NC(=O)C(=O)NCc1ccccc1C(=O)NC1(c2cccc3ccccc23)CC1] |
| 157 | 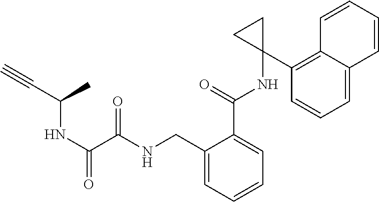 [SMILES: C#C[C@@H](C)NC(=O)C(=O)NCc1ccccc1C(=O)NC1(c2cccc3ccccc23)CC1] |
| 158 | 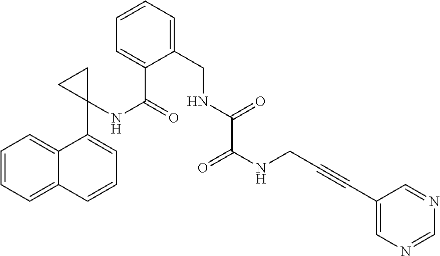 [SMILES: O=C(NCC#Cc1cncnc1)C(=O)NCc1ccccc1C(=O)NC1(c2cccc3ccccc23)CC1] |
| 159 | 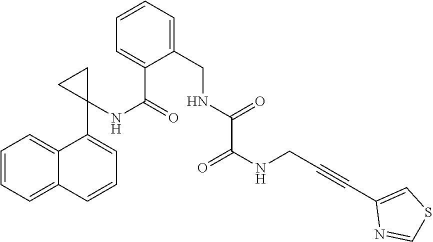 [SMILES: O=C(NCC#Cc1cscn1)C(=O)NCc1ccccc1C(=O)NC1(c2cccc3ccccc23)CC1] |
| 160 | 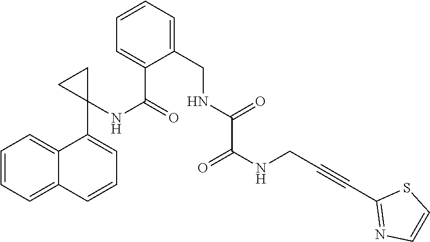 [SMILES: O=C(NCC#Cc1nccs1)C(=O)NCc1ccccc1C(=O)NC1(c2cccc3ccccc23)CC1] |
| 161 | 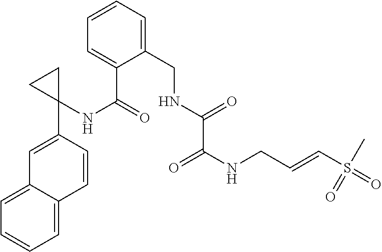 [SMILES: CS(=O)(=O)/C=C/CNC(=O)C(=O)NCc1ccccc1C(=O)NC1(c2ccc3ccccc3c2)CC1] |
| 162 | 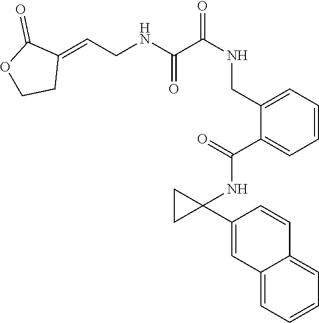 [SMILES: O=C(NC/C=C1\CCOC1=O)C(=O)NCc1ccccc1C(=O)NC1(c2ccc3ccccc3c2)CC1] |
| 163 | 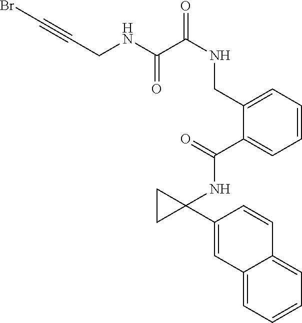 [SMILES: O=C(NCC#CBr)C(=O)NCc1ccccc1C(=O)NC1(c2ccc3ccccc3c2)CC1] |
| 164 | 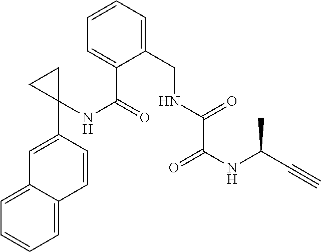 [SMILES: C#C[C@H](C)NC(=O)C(=O)NCc1ccccc1C(=O)NC1(c2ccc3ccccc3c2)CC1] |
| 165 | 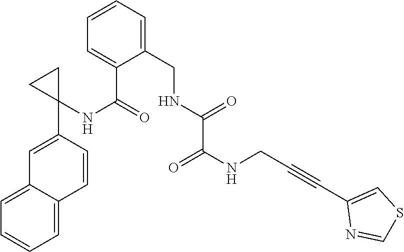 [SMILES: O=C(NCC#Cc1cscn1)C(=O)NCc1ccccc1C(=O)NC1(c2ccc3ccccc3c2)CC1] |
| 166 | 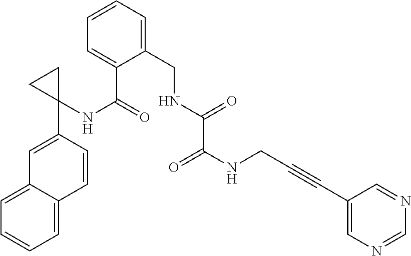 [SMILES: O=C(NCC#Cc1cncnc1)C(=O)NCc1ccccc1C(=O)NC1(c2ccc3ccccc3c2)CC1] |
| 167 | 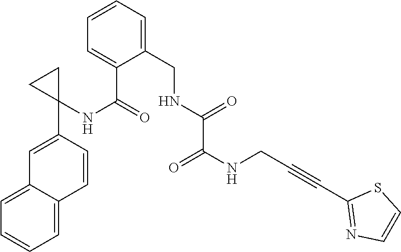 [SMILES: O=C(NCC#Cc1nccs1)C(=O)NCc1ccccc1C(=O)NC1(c2ccc3ccccc3c2)CC1] |
| 168 | 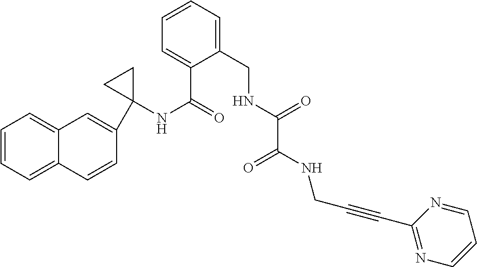 [SMILES: O=C(NCC#Cc1ncccn1)C(=O)NCc1ccccc1C(=O)NC1(c2ccc3ccccc3c2)CC1] |
| 169 | 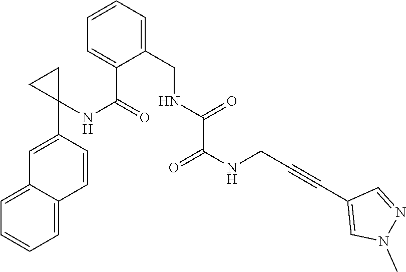 [SMILES: Cn1cc(C#CCNC(=O)C(=O)NCc2ccccc2C(=O)NC2(c3ccc4ccccc4c3)CC2)cn1] |
| 170 | 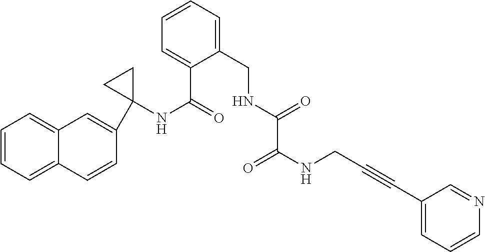 [SMILES: O=C(NCC#Cc1cccnc1)C(=O)NCc1ccccc1C(=O)NC1(c2ccc3ccccc3c2)CC1] |
| 171 | 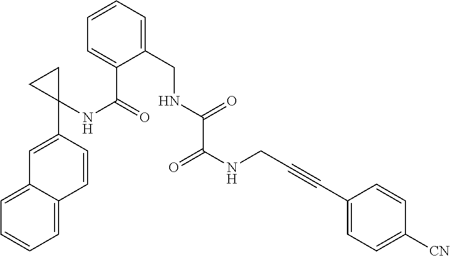 [SMILES: N#Cc1ccc(C#CCNC(=O)C(=O)NCc2ccccc2C(=O)NC2(c3ccc4ccccc4c3)CC2)cc1] |
| 172 | 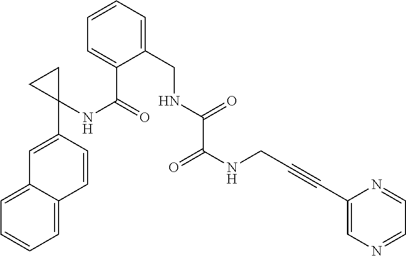 [SMILES: O=C(NCC#Cc1cnccn1)C(=O)NCc1ccccc1C(=O)NC1(c2ccc3ccccc3c2)CC1] |
| 173 | 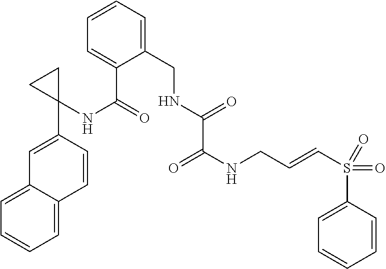 [SMILES: O=C(NC/C=C/S(=O)(=O)c1ccccc1)C(=O)NCc1ccccc1C(=O)NC1(c2ccc3ccccc3c2)CC1] |
| 174 | 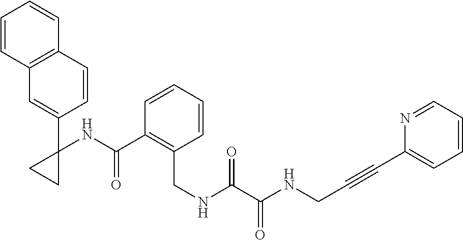 [SMILES: O=C(NCC#Cc1ccccn1)C(=O)NCc1ccccc1C(=O)NC1(c2ccc3ccccc3c2)CC1] |
| 175 | 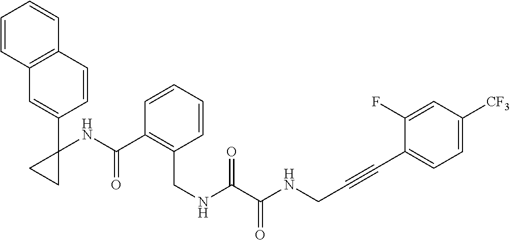 [SMILES: O=C(NCC#Cc1ccc(C(F)(F)F)cc1F)C(=O)NCc1ccccc1C(=O)NC1(c2ccc3ccccc3c2)CC1] |
| 176 | 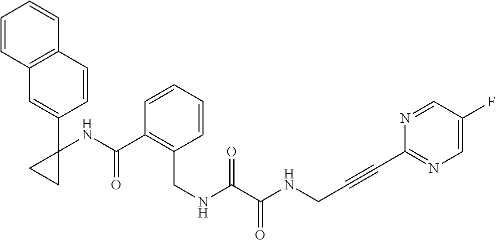 [SMILES: O=C(NCC#Cc1ncc(F)cn1)C(=O)NCc1ccccc1C(=O)NC1(c2ccc3ccccc3c2)CC1] |
| 177 | 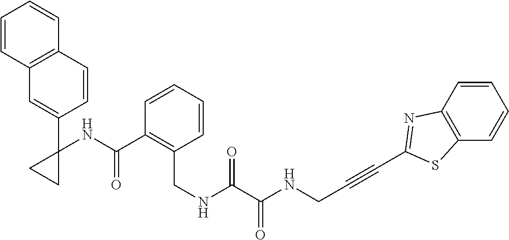 [SMILES: O=C(NCC#Cc1nc2ccccc2s1)C(=O)NCc1ccccc1C(=O)NC1(c2ccc3ccccc3c2)CC1] |
| 178 | 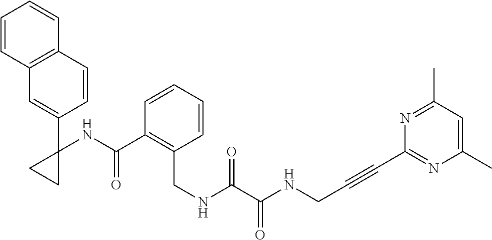 [SMILES: Cc1cc(C)nc(C#CCNC(=O)C(=O)NCc2ccccc2C(=O)NC2(c3ccc4ccccc4c3)CC2)n1] |
| 179 | 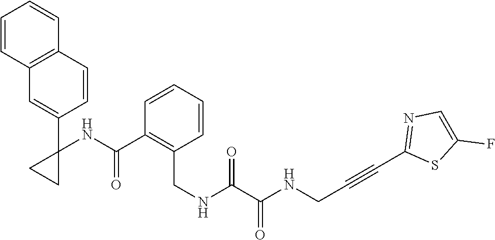 [SMILES: O=C(NCC#Cc1ncc(F)s1)C(=O)NCc1ccccc1C(=O)NC1(c2ccc3ccccc3c2)CC1] |
| 180 | 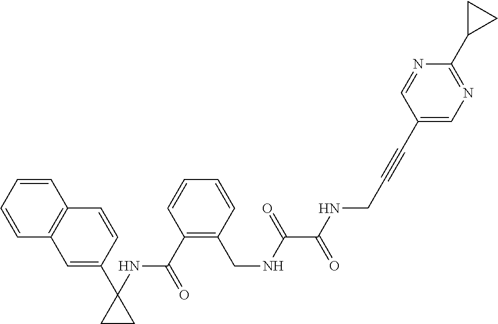 [SMILES: O=C(NCC#Cc1cnc(C2CC2)nc1)C(=O)NCc1ccccc1C(=O)NC1(c2ccc3ccccc3c2)CC1] |
| 181 | 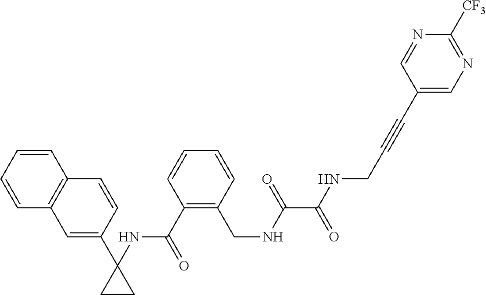 [SMILES: O=C(NCC#Cc1cnc(C(F)(F)F)nc1)C(=O)NCc1ccccc1C(=O)NC1(c2ccc3ccccc3c2)CC1] |
| 182 | 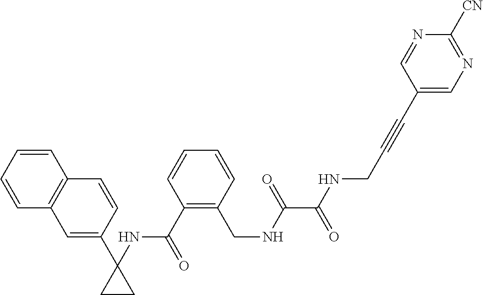 [SMILES: N#Cc1ncc(C#CCNC(=O)C(=O)NCc2ccccc2C(=O)NC2(c3ccc4ccccc4c3)CC2)cn1] |
| 183 | 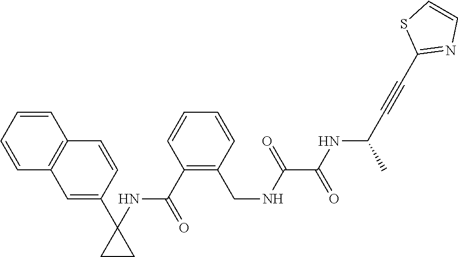 [SMILES: C[C@@H](C#Cc1nccs1)NC(=O)C(=O)NCc1ccccc1C(=O)NC1(c2ccc3ccccc3c2)CC1] |
| 184 | 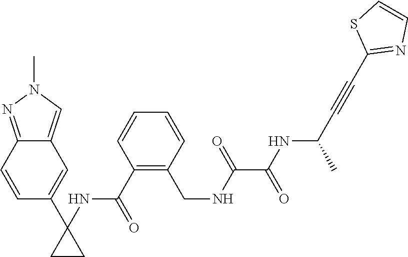 [SMILES: C[C@@H](C#Cc1nccs1)NC(=O)C(=O)NCc1ccccc1C(=O)NC1(c2ccc3nn(C)cc3c2)CC1] |
| 185 | 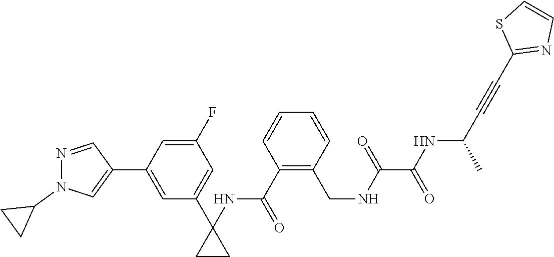 [SMILES: C[C@@H](C#Cc1nccs1)NC(=O)C(=O)NCc1ccccc1C(=O)NC1(c2cc(F)cc(-c3cnn(C4CC4)c3)c2)CC1] |
| 186 | 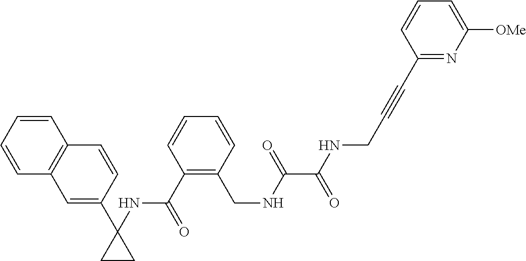 [SMILES: COc1cccc(C#CCNC(=O)C(=O)NCc2ccccc2C(=O)NC2(c3ccc4ccccc4c3)CC2)n1] |
| 187 | 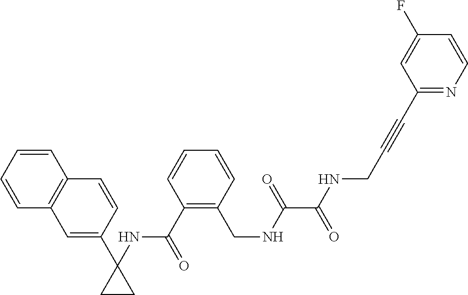 [SMILES: O=C(NCC#Cc1cc(F)ccn1)C(=O)NCc1ccccc1C(=O)NC1(c2ccc3ccccc3c2)CC1] |
| 188 | 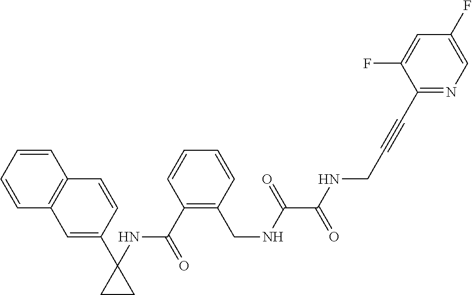 [SMILES: O=C(NCC#Cc1ncc(F)cc1F)C(=O)NCc1ccccc1C(=O)NC1(c2ccc3ccccc3c2)CC1] |
| 189 | 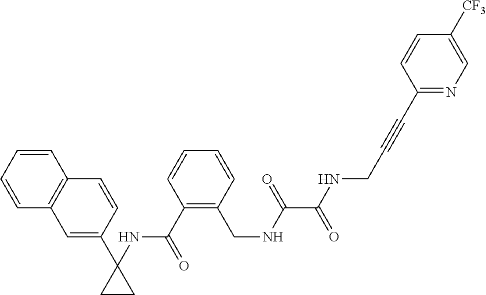 [SMILES: O=C(NCC#Cc1ccc(C(F)(F)F)cn1)C(=O)NCc1ccccc1C(=O)NC1(c2ccc3ccccc3c2)CC1] |
| 190 | 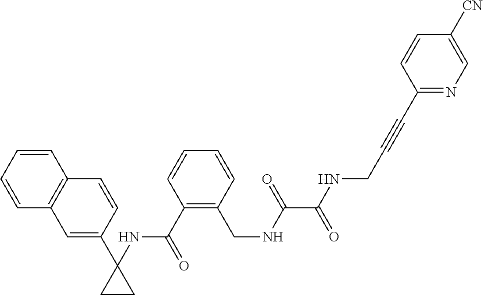 [SMILES: N#Cc1ccc(C#CCNC(=O)C(=O)NCc2ccccc2C(=O)NC2(c3ccc4ccccc4c3)CC2)nc1] |
| 191 | 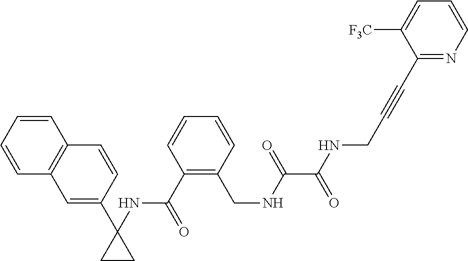 [SMILES: O=C(NCC#Cc1ncccc1C(F)(F)F)C(=O)NCc1ccccc1C(=O)NC1(c2ccc3ccccc3c2)CC1] |
| 192 | 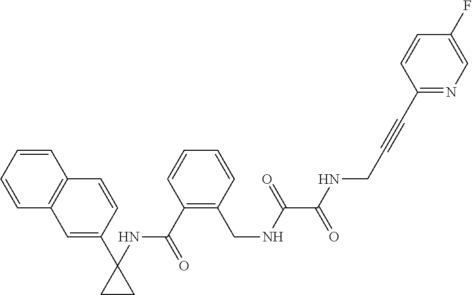 [SMILES: O=C(NCC#Cc1ccc(F)cn1)C(=O)NCc1ccccc1C(=O)NC1(c2ccc3ccccc3c2)CC1] |
| 193 | 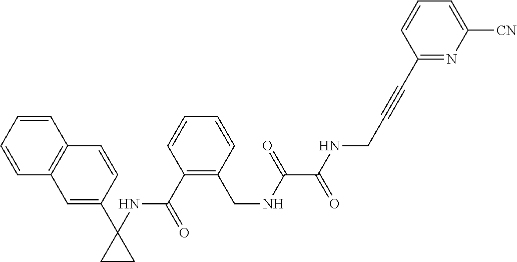 [SMILES: N#Cc1cccc(C#CCNC(=O)C(=O)NCc2ccccc2C(=O)NC2(c3ccc4ccccc4c3)CC2)n1] |
| 194 | 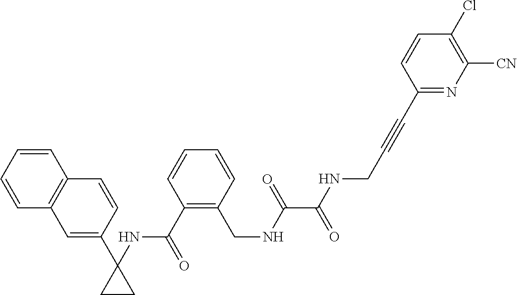 [SMILES: N#Cc1nc(C#CCNC(=O)C(=O)NCc2ccccc2C(=O)NC2(c3ccc4ccccc4c3)CC2)ccc1Cl] |
| 195 | 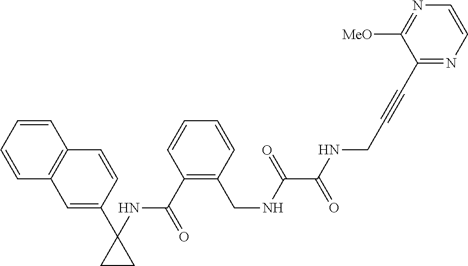 [SMILES: COc1nccnc1C#CCNC(=O)C(=O)NCc1ccccc1C(=O)NC1(c2ccc3ccccc3c2)CC1] |
| 196 | 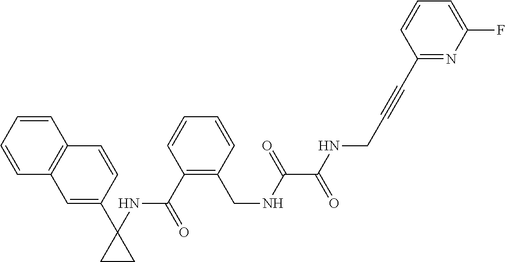 [SMILES: O=C(NCC#Cc1cccc(F)n1)C(=O)NCc1ccccc1C(=O)NC1(c2ccc3ccccc3c2)CC1] |
| 197 | 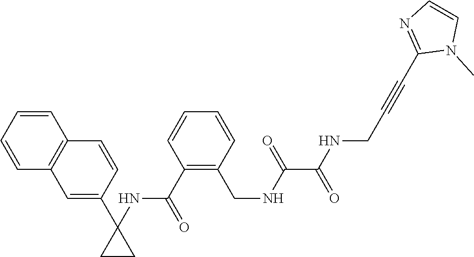 [SMILES: Cn1ccnc1C#CCNC(=O)C(=O)NCc1ccccc1C(=O)NC1(c2ccc3ccccc3c2)CC1] |
| 198 | 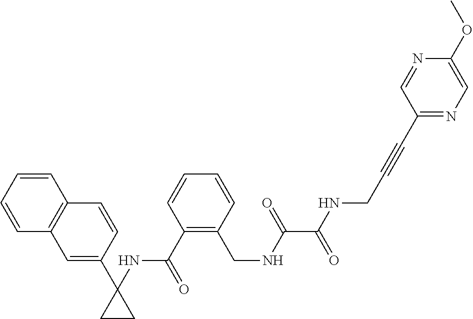 [SMILES: COc1cnc(C#CCNC(=O)C(=O)NCc2ccccc2C(=O)NC2(c3ccc4ccccc4c3)CC2)cn1] |
| 199 | 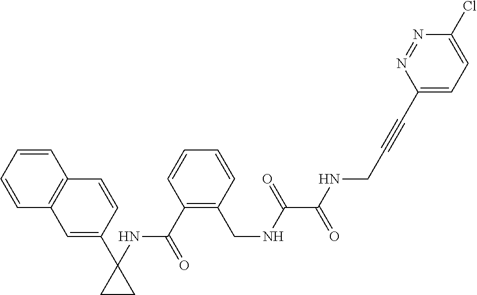 [SMILES: O=C(NCC#Cc1ccc(Cl)nn1)C(=O)NCc1ccccc1C(=O)NC1(c2ccc3ccccc3c2)CC1] |
| 200 | 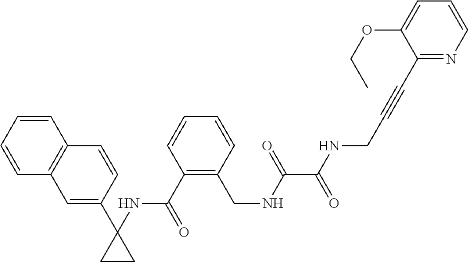 [SMILES: CCOc1cccnc1C#CCNC(=O)C(=O)NCc1ccccc1C(=O)NC1(c2ccc3ccccc3c2)CC1] |
| 201 | 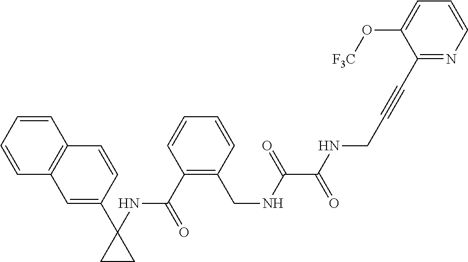 [SMILES: O=C(NCC#Cc1ncccc1OC(F)(F)F)C(=O)NCc1ccccc1C(=O)NC1(c2ccc3ccccc3c2)CC1] |
| 202 | 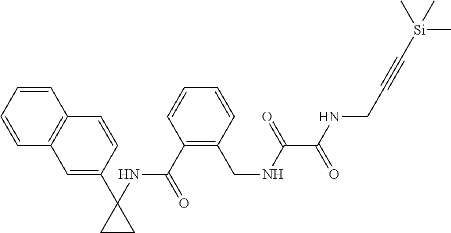 [SMILES: C[Si](C)(C)C#CCNC(=O)C(=O)NCc1ccccc1C(=O)NC1(c2ccc3ccccc3c2)CC1] |
| 203 | 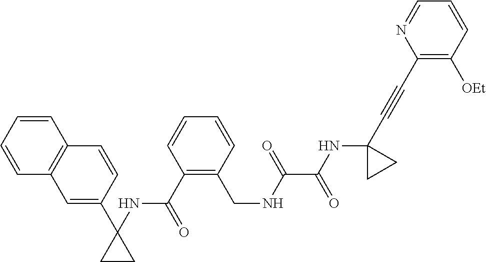 [SMILES: CCOc1cccnc1C#CC1(NC(=O)C(=O)NCc2ccccc2C(=O)NC2(c3ccc4ccccc4c3)CC2)CC1] |
| 204 | 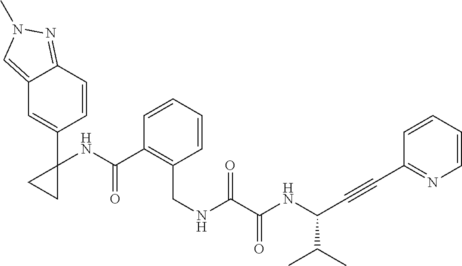 [SMILES: CC(C)[C@@H](C#Cc1ccccn1)NC(=O)C(=O)NCc1ccccc1C(=O)NC1(c2ccc3nn(C)cc3c2)CC1] |
| 205 | 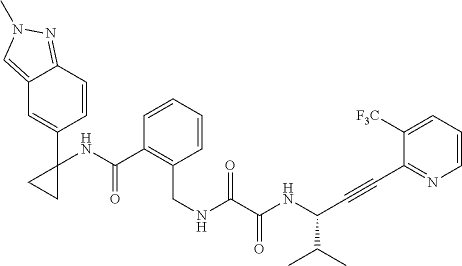 [SMILES: CC(C)[C@@H](C#Cc1ncccc1C(F)(F)F)NC(=O)C(=O)NCc1ccccc1C(=O)NC1(c2ccc3nn(C)cc3c2)CC1] |
| 206 | 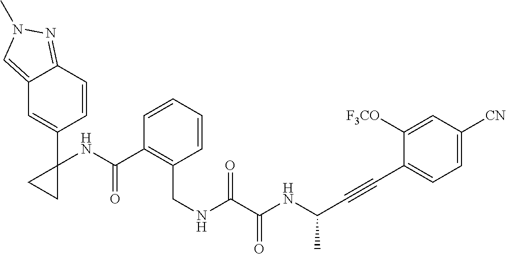 [SMILES: C[C@@H](C#Cc1ccc(C#N)cc1OC(F)(F)F)NC(=O)C(=O)NCc1ccccc1C(=O)NC1(c2ccc3nn(C)cc3c2)CC1] |
| 207 | 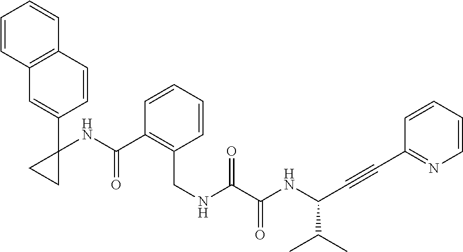 [SMILES: CC(C)[C@@H](C#Cc1ccccn1)NC(=O)C(=O)NCc1ccccc1C(=O)NC1(c2ccc3ccccc3c2)CC1] |
| 208 | 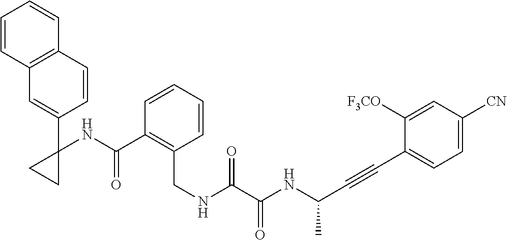 [SMILES: C[C@@H](C#Cc1ccc(C#N)cc1OC(F)(F)F)NC(=O)C(=O)NCc1ccccc1C(=O)NC1(c2ccc3ccccc3c2)CC1] |
| 209 | 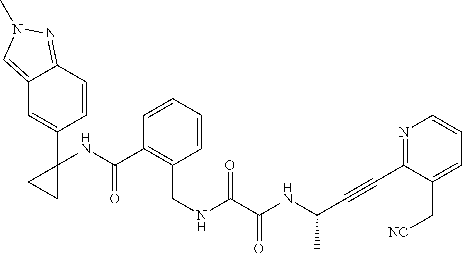 [SMILES: C[C@@H](C#Cc1ncccc1CC#N)NC(=O)C(=O)NCc1ccccc1C(=O)NC1(c2ccc3nn(C)cc3c2)CC1] |
| 210 | 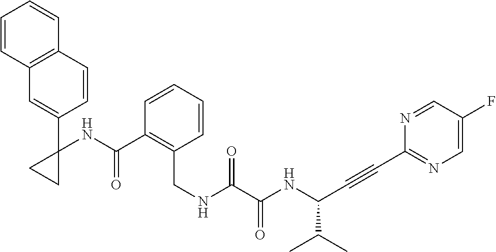 [SMILES: CC(C)[C@@H](C#Cc1ncc(F)cn1)NC(=O)C(=O)NCc1ccccc1C(=O)NC1(c2ccc3ccccc3c2)CC1] |
| 211 | 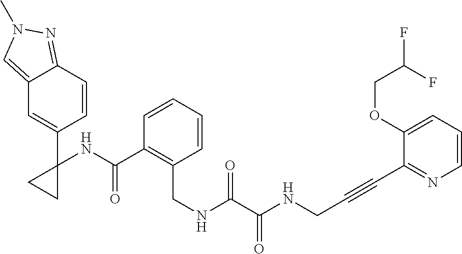 [SMILES: Cn1cc2cc(C3(NC(=O)c4ccccc4CNC(=O)C(=O)NCC#Cc4ncccc4OCC(F)F)CC3)ccc2n1] |
| 212 | 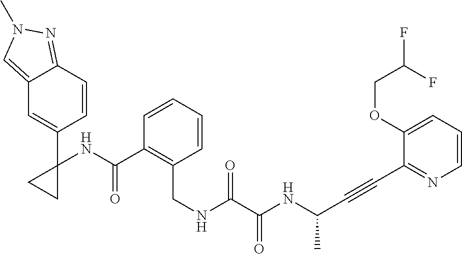 [SMILES: C[C@@H](C#Cc1ncccc1OCC(F)F)NC(=O)C(=O)NCc1ccccc1C(=O)NC1(c2ccc3nn(C)cc3c2)CC1] |
| 213 | 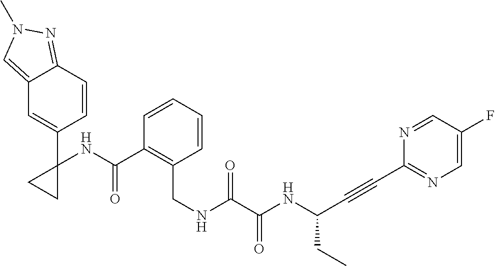 [SMILES: CC[C@@H](C#Cc1ncc(F)cn1)NC(=O)C(=O)NCc1ccccc1C(=O)NC1(c2ccc3nn(C)cc3c2)CC1] |
| 214 | 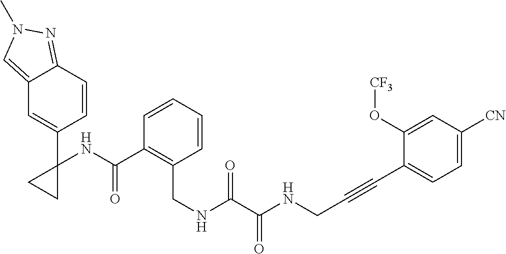 [SMILES: Cn1cc2cc(C3(NC(=O)c4ccccc4CNC(=O)C(=O)NCC#Cc4ccc(C#N)cc4OC(F)(F)F)CC3)ccc2n1] |
| 215 | 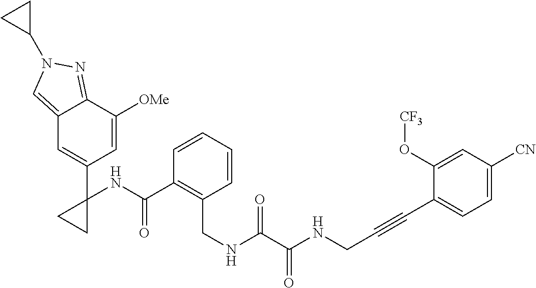 [SMILES: COc1cc(C2(NC(=O)c3ccccc3CNC(=O)C(=O)NCC#Cc3ccc(C#N)cc3OC(F)(F)F)CC2)cc2cn(C3CC3)nc12] |
| 216 | 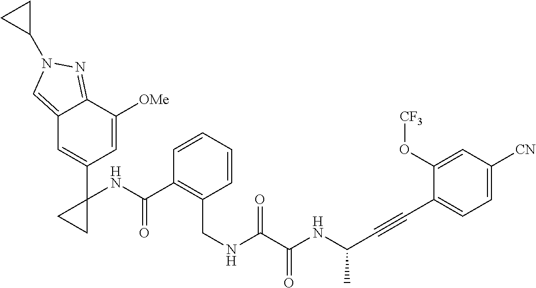 [SMILES: COc1cc(C2(NC(=O)c3ccccc3CNC(=O)C(=O)N[C@@H](C)C#Cc3ccc(C#N)cc3OC(F)(F)F)CC2)cc2cn(C3CC3)nc12] |
| 217 | 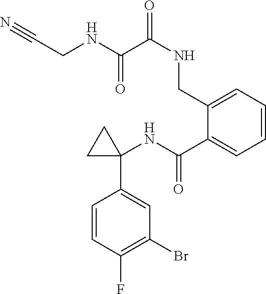 [SMILES: N#CCNC(=O)C(=O)NCc1ccccc1C(=O)NC1(c2ccc(F)c(Br)c2)CC1] |
| 218 | 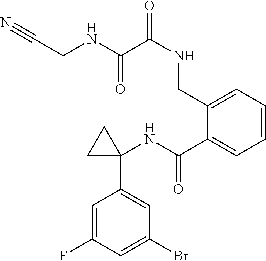 [SMILES: N#CCNC(=O)C(=O)NCc1ccccc1C(=O)NC1(c2cc(F)cc(Br)c2)CC1] |
| 219 | 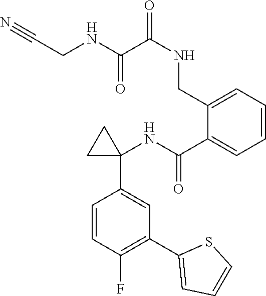 [SMILES: N#CCNC(=O)C(=O)NCc1ccccc1C(=O)NC1(c2ccc(F)c(-c3cccs3)c2)CC1] |
| 220 | 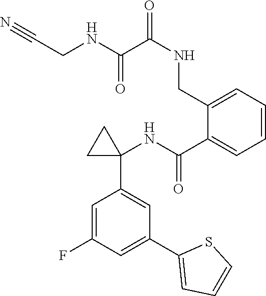 [SMILES: N#CCNC(=O)C(=O)NCc1ccccc1C(=O)NC1(c2cc(F)cc(-c3cccs3)c2)CC1] |
| 221 | 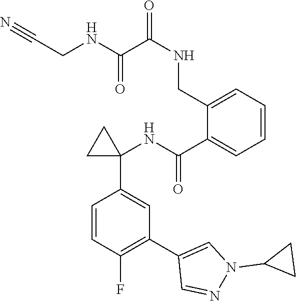 [SMILES: N#CCNC(=O)C(=O)NCc1ccccc1C(=O)NC1(c2ccc(F)c(-c3cnn(C4CC4)c3)c2)CC1] |
| 222 | 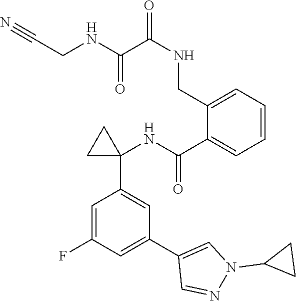 [SMILES: N#CCNC(=O)C(=O)NCc1ccccc1C(=O)NC1(c2cc(F)cc(-c3cnn(C4CC4)c3)c2)CC1] |
| 223 | 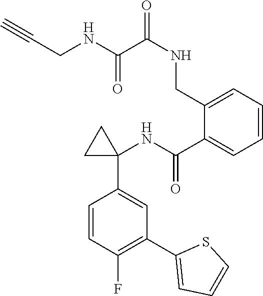 [SMILES: C#CCNC(=O)C(=O)NCc1ccccc1C(=O)NC1(c2ccc(F)c(-c3cccs3)c2)CC1] |
| 224 | 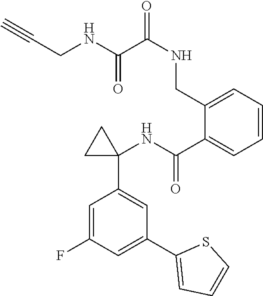 [SMILES: C#CCNC(=O)C(=O)NCc1ccccc1C(=O)NC1(c2cc(F)cc(-c3cccs3)c2)CC1] |
| 225 | 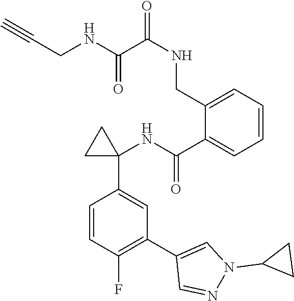 [SMILES: C#CCNC(=O)C(=O)NCc1ccccc1C(=O)NC1(c2ccc(F)c(-c3cnn(C4CC4)c3)c2)CC1] |
| 226 | 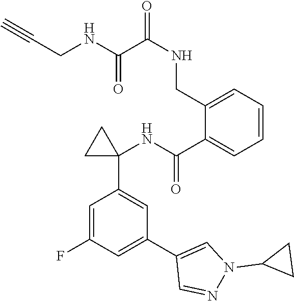 [SMILES: C#CCNC(=O)C(=O)NCc1ccccc1C(=O)NC1(c2cc(F)cc(-c3cnn(C4CC4)c3)c2)CC1] |
| 227 | 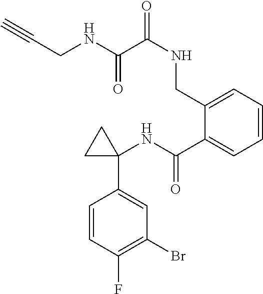 [SMILES: C#CCNC(=O)C(=O)NCc1ccccc1C(=O)NC1(c2ccc(F)c(Br)c2)CC1] |
| 228 | 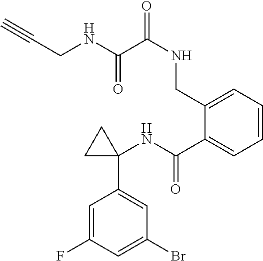 [SMILES: C#CCNC(=O)C(=O)NCc1ccccc1C(=O)NC1(c2cc(F)cc(Br)c2)CC1] |
| 229 | 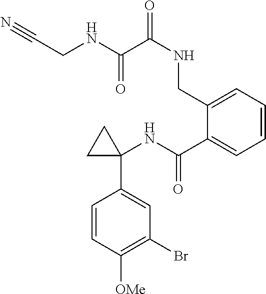 [SMILES: COc1ccc(C2(NC(=O)c3ccccc3CNC(=O)C(=O)NCC#N)CC2)cc1Br] |
| 230 | 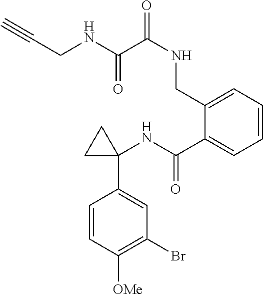 [SMILES: C#CCNC(=O)C(=O)NCc1ccccc1C(=O)NC1(c2ccc(OC)c(Br)c2)CC1] |
| 231 | 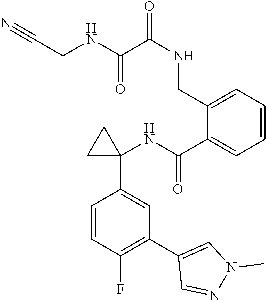 [SMILES: Cn1cc(-c2cc(C3(NC(=O)c4ccccc4CNC(=O)C(=O)NCC#N)CC3)ccc2F)cn1] |
| 232 | 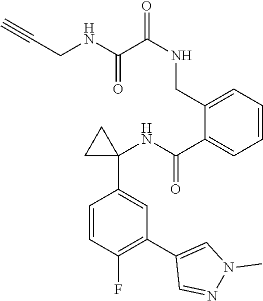 [SMILES: C#CCNC(=O)C(=O)NCc1ccccc1C(=O)NC1(c2ccc(F)c(-c3cnn(C)c3)c2)CC1] |
| 233 | 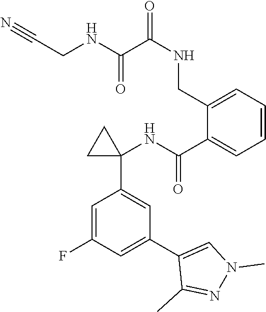 [SMILES: Cc1nn(C)cc1-c1cc(F)cc(C2(NC(=O)c3ccccc3CNC(=O)C(=O)NCC#N)CC2)c1] |
| 234 | 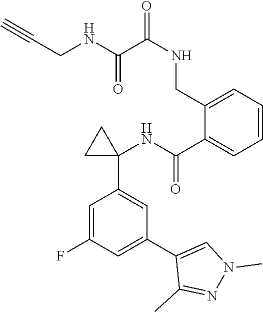 [SMILES: C#CCNC(=O)C(=O)NCc1ccccc1C(=O)NC1(c2cc(F)cc(-c3cn(C)nc3C)c2)CC1] |
| 235 | 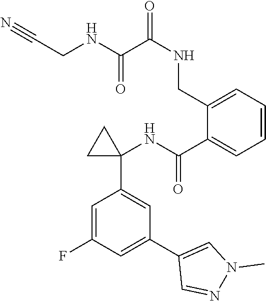 [SMILES: Cn1cc(-c2cc(F)cc(C3(NC(=O)c4ccccc4CNC(=O)C(=O)NCC#N)CC3)c2)cn1] |
| 236 | 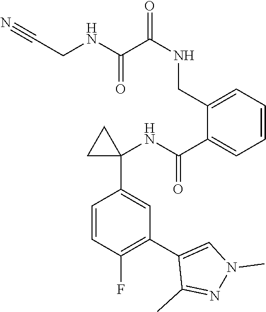 [SMILES: Cc1nn(C)cc1-c1cc(C2(NC(=O)c3ccccc3CNC(=O)C(=O)NCC#N)CC2)ccc1F] |
| 237 | 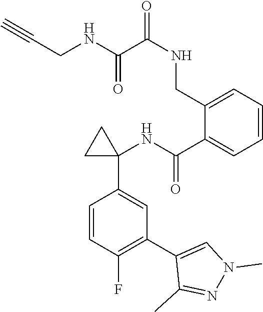 [SMILES: C#CCNC(=O)C(=O)NCc1ccccc1C(=O)NC1(c2ccc(F)c(-c3cn(C)nc3C)c2)CC1] |
| 238 | 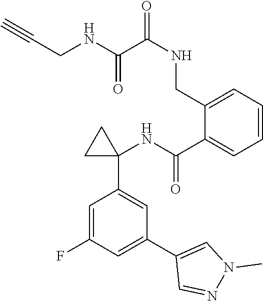 [SMILES: C#CCNC(=O)C(=O)NCc1ccccc1C(=O)NC1(c2cc(F)cc(-c3cnn(C)c3)c2)CC1] |
| 239 | 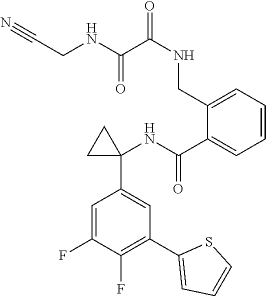 [SMILES: N#CCNC(=O)C(=O)NCc1ccccc1C(=O)NC1(c2cc(F)c(F)c(-c3cccs3)c2)CC1] |
| 240 | 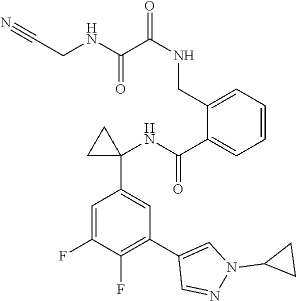 [SMILES: N#CCNC(=O)C(=O)NCc1ccccc1C(=O)NC1(c2cc(F)c(F)c(-c3cnn(C4CC4)c3)c2)CC1] |
| 241 | 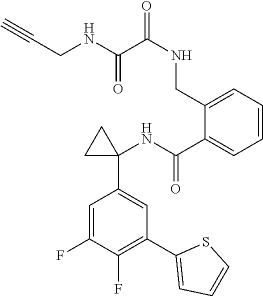 [SMILES: C#CCNC(=O)C(=O)NCc1ccccc1C(=O)NC1(c2cc(F)c(F)c(-c3cccs3)c2)CC1] |
| 242 | 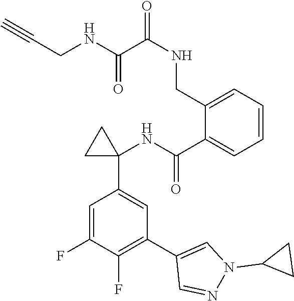 [SMILES: C#CCNC(=O)C(=O)NCc1ccccc1C(=O)NC1(c2cc(F)c(F)c(-c3cnn(C4CC4)c3)c2)CC1] |
| 243 | 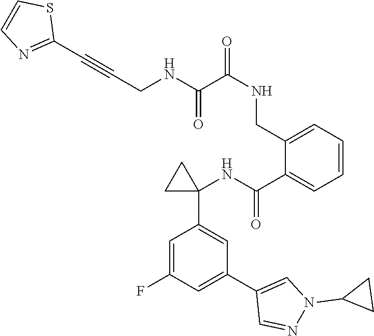 [SMILES: O=C(NCC#Cc1nccs1)C(=O)NCc1ccccc1C(=O)NC1(c2cc(F)cc(-c3cnn(C4CC4)c3)c2)CC1] |
| 244 | 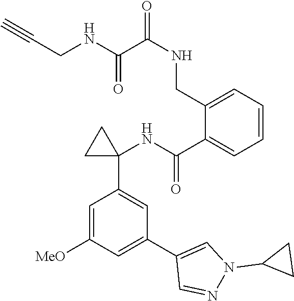 [SMILES: C#CCNC(=O)C(=O)NCc1ccccc1C(=O)NC1(c2cc(OC)cc(-c3cnn(C4CC4)c3)c2)CC1] |
| 245 | 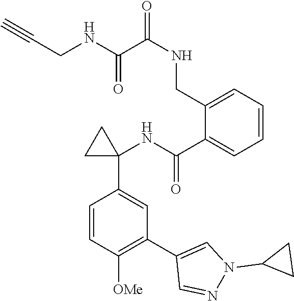 [SMILES: C#CCNC(=O)C(=O)NCc1ccccc1C(=O)NC1(c2ccc(OC)c(-c3cnn(C4CC4)c3)c2)CC1] |
| 246 | 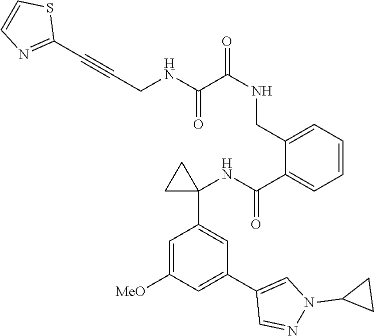 [SMILES: COc1cc(-c2cnn(C3CC3)c2)cc(C2(NC(=O)c3ccccc3CNC(=O)C(=O)NCC#Cc3nccs3)CC2)c1] |
| 247 | 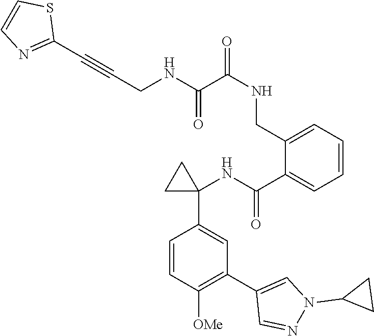 [SMILES: COc1ccc(C2(NC(=O)c3ccccc3CNC(=O)C(=O)NCC#Cc3nccs3)CC2)cc1-c1cnn(C2CC2)c1] |
| 248 | 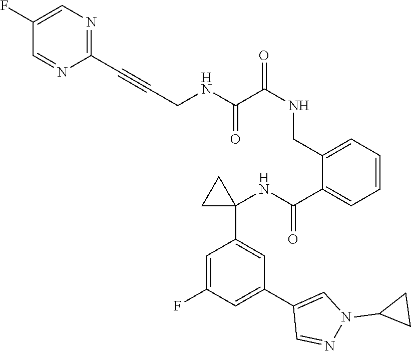 [SMILES: O=C(NCC#Cc1ncc(F)cn1)C(=O)NCc1ccccc1C(=O)NC1(c2cc(F)cc(-c3cnn(C4CC4)c3)c2)CC1] |
| 249 | 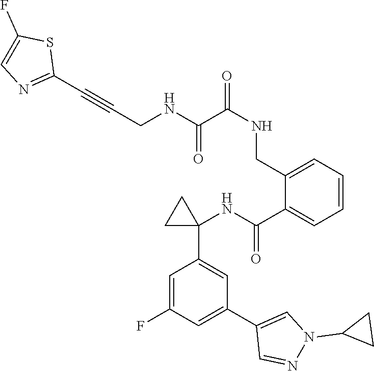 [SMILES: O=C(NCC#Cc1ncc(F)s1)C(=O)NCc1ccccc1C(=O)NC1(c2cc(F)cc(-c3cnn(C4CC4)c3)c2)CC1] |
| 250 | 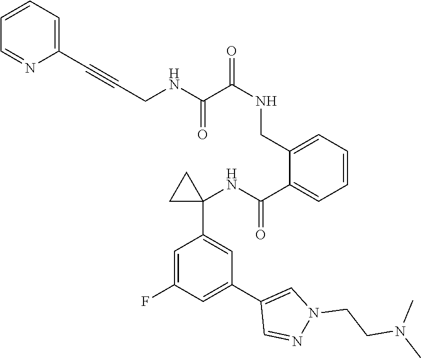 [SMILES: CN(C)CCn1cc(-c2cc(F)cc(C3(NC(=O)c4ccccc4CNC(=O)C(=O)NCC#Cc4ccccn4)CC3)c2)cn1] |
| 251 | 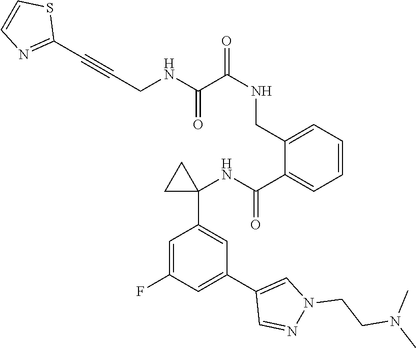 [SMILES: CN(C)CCn1cc(-c2cc(F)cc(C3(NC(=O)c4ccccc4CNC(=O)C(=O)NCC#Cc4nccs4)CC3)c2)cn1] |
| 252 | 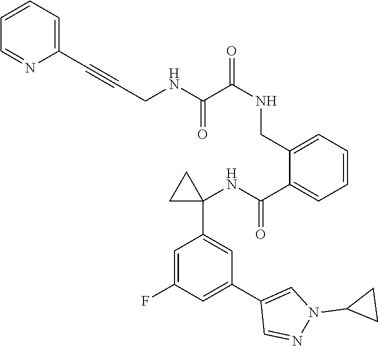 [SMILES: O=C(NCC#Cc1ccccn1)C(=O)NCc1ccccc1C(=O)NC1(c2cc(F)cc(-c3cnn(C4CC4)c3)c2)CC1] |
| 253 | 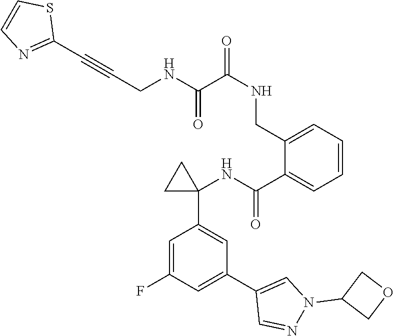 [SMILES: O=C(NCC#Cc1nccs1)C(=O)NCc1ccccc1C(=O)NC1(c2cc(F)cc(-c3cnn(C4COC4)c3)c2)CC1] |
| 254 | 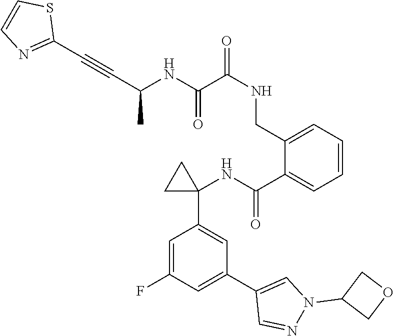 [SMILES: C[C@@H](C#Cc1nccs1)NC(=O)C(=O)NCc1ccccc1C(=O)NC1(c2cc(F)cc(-c3cnn(C4COC4)c3)c2)CC1] |
| 255 | 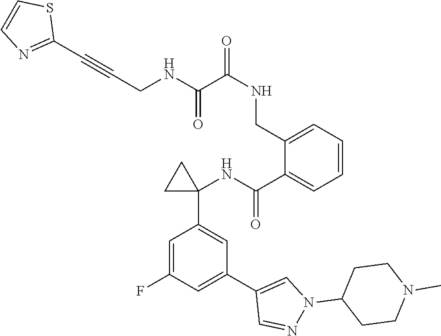 [SMILES: CN1CCC(n2cc(-c3cc(F)cc(C4(NC(=O)c5ccccc5CNC(=O)C(=O)NCC#Cc5nccs5)CC4)c3)cn2)CC1] |
| 256 | 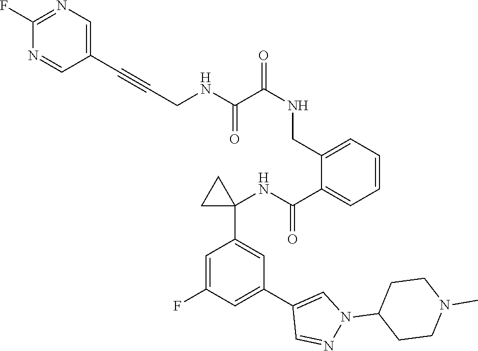 [SMILES: CN1CCC(n2cc(-c3cc(F)cc(C4(NC(=O)c5ccccc5CNC(=O)C(=O)NCC#Cc5cnc(F)nc5)CC4)c3)cn2)CC1] |
| 257 | 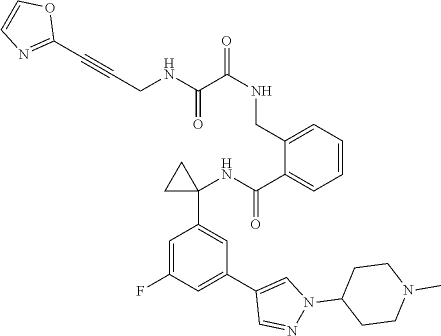 [SMILES: CN1CCC(n2cc(-c3cc(F)cc(C4(NC(=O)c5ccccc5CNC(=O)C(=O)NCC#Cc5ncco5)CC4)c3)cn2)CC1] |
| 258 | 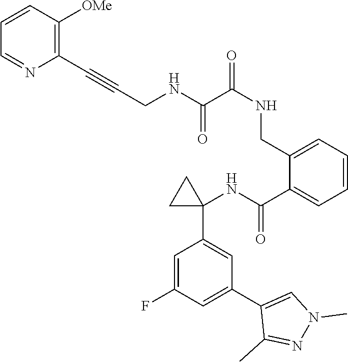 [SMILES: COc1cccnc1C#CCNC(=O)C(=O)NCc1ccccc1C(=O)NC1(c2cc(F)cc(-c3cn(C)nc3C)c2)CC1] |
| 259 | 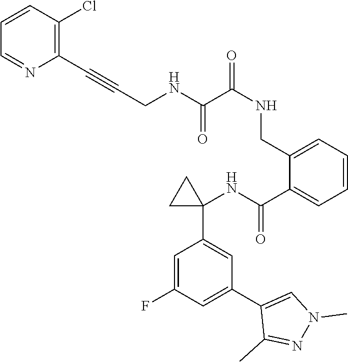 [SMILES: Cc1nn(C)cc1-c1cc(F)cc(C2(NC(=O)c3ccccc3CNC(=O)C(=O)NCC#Cc3ncccc3Cl)CC2)c1] |
| 260 | 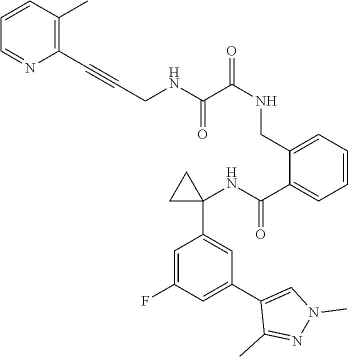 [SMILES: Cc1cccnc1C#CCNC(=O)C(=O)NCc1ccccc1C(=O)NC1(c2cc(F)cc(-c3cn(C)nc3C)c2)CC1] |
| 261 | 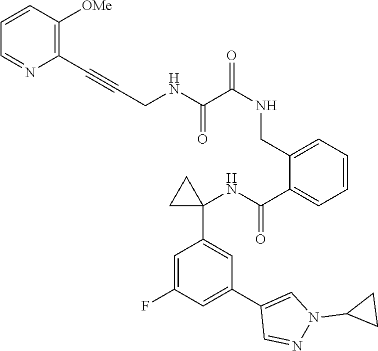 [SMILES: COc1cccnc1C#CCNC(=O)C(=O)NCc1ccccc1C(=O)NC1(c2cc(F)cc(-c3cnn(C4CC4)c3)c2)CC1] |
| 262 | 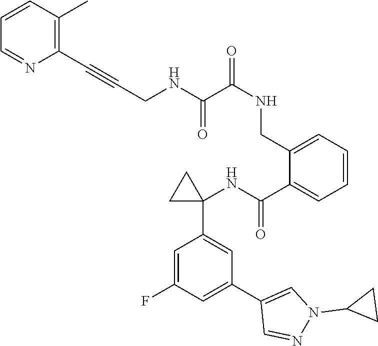 [SMILES: Cc1cccnc1C#CCNC(=O)C(=O)NCc1ccccc1C(=O)NC1(c2cc(F)cc(-c3cnn(C4CC4)c3)c2)CC1] |
| 263 | 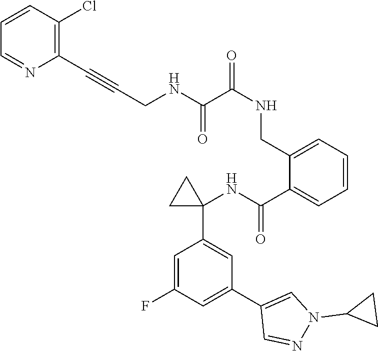 [SMILES: O=C(NCC#Cc1ncccc1Cl)C(=O)NCc1ccccc1C(=O)NC1(c2cc(F)cc(-c3cnn(C4CC4)c3)c2)CC1] |
| 264 | 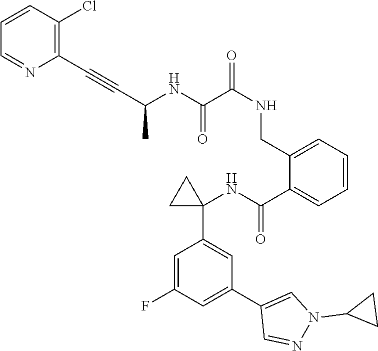 [SMILES: C[C@@H](C#Cc1ncccc1Cl)NC(=O)C(=O)NCc1ccccc1C(=O)NC1(c2cc(F)cc(-c3cnn(C4CC4)c3)c2)CC1] |
| 265 | 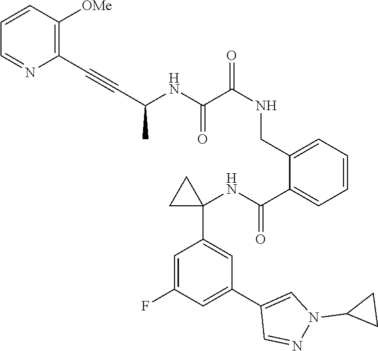 [SMILES: COc1cccnc1C#C[C@H](C)NC(=O)C(=O)NCc1ccccc1C(=O)NC1(c2cc(F)cc(-c3cnn(C4CC4)c3)c2)CC1] |
| 266 | 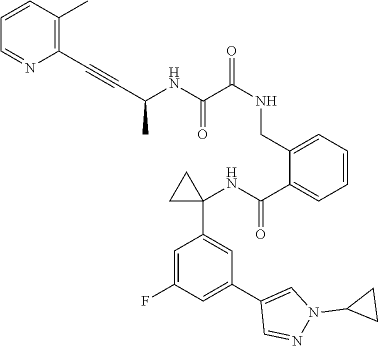 [SMILES: Cc1cccnc1C#C[C@H](C)NC(=O)C(=O)NCc1ccccc1C(=O)NC1(c2cc(F)cc(-c3cnn(C4CC4)c3)c2)CC1] |
| 267 | 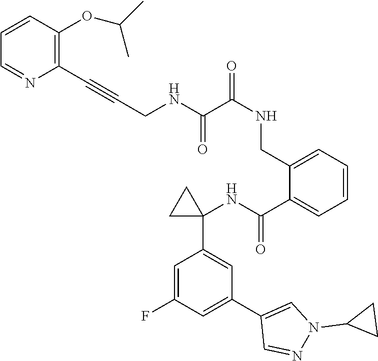 [SMILES: CC(C)Oc1cccnc1C#CCNC(=O)C(=O)NCc1ccccc1C(=O)NC1(c2cc(F)cc(-c3cnn(C4CC4)c3)c2)CC1] |
| 268 | 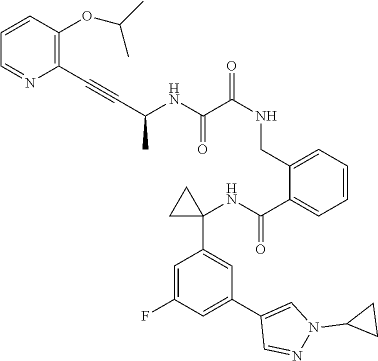 [SMILES: CC(C)Oc1cccnc1C#C[C@H](C)NC(=O)C(=O)NCc1ccccc1C(=O)NC1(c2cc(F)cc(-c3cnn(C4CC4)c3)c2)CC1] |
| 269 | 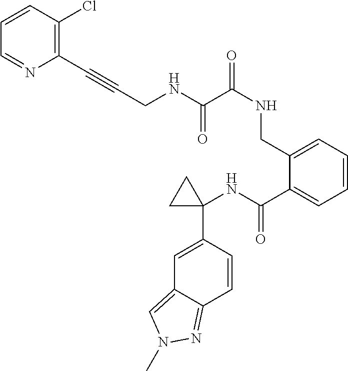 [SMILES: Cn1cc2cc(C3(NC(=O)c4ccccc4CNC(=O)C(=O)NCC#Cc4ncccc4Cl)CC3)ccc2n1] |
| 270 | 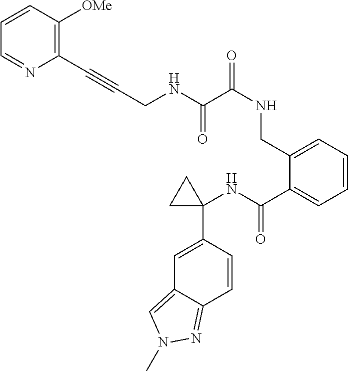 [SMILES: COc1cccnc1C#CCNC(=O)C(=O)NCc1ccccc1C(=O)NC1(c2ccc3nn(C)cc3c2)CC1] |
| 271 | 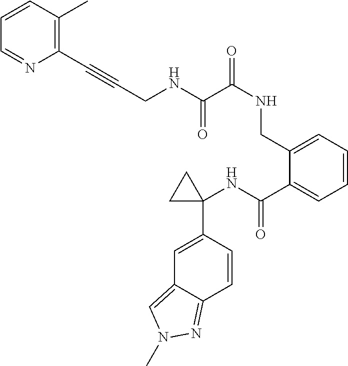 [SMILES: Cc1cccnc1C#CCNC(=O)C(=O)NCc1ccccc1C(=O)NC1(c2ccc3nn(C)cc3c2)CC1] |
| 272 | 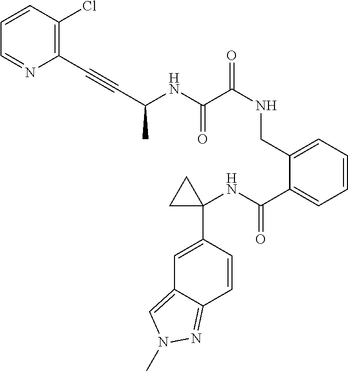 [SMILES: C[C@@H](C#Cc1ncccc1Cl)NC(=O)C(=O)NCc1ccccc1C(=O)NC1(c2ccc3nn(C)cc3c2)CC1] |
| 273 | 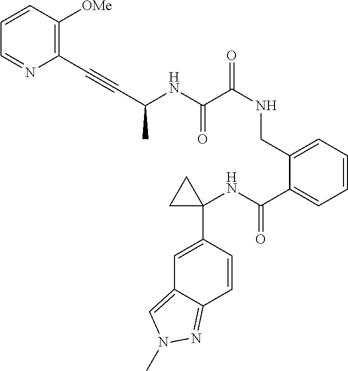 [SMILES: COc1cccnc1C#C[C@H](C)NC(=O)C(=O)NCc1ccccc1C(=O)NC1(c2ccc3nn(C)cc3c2)CC1] |
| 274 | 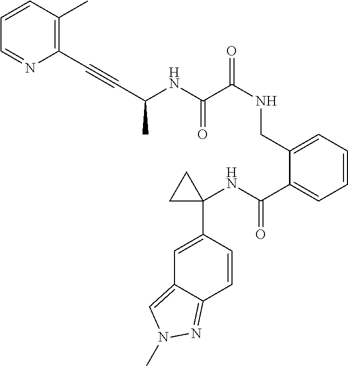 [SMILES: Cc1cccnc1C#C[C@H](C)NC(=O)C(=O)NCc1ccccc1C(=O)NC1(c2ccc3nn(C)cc3c2)CC1] |
| 275 | 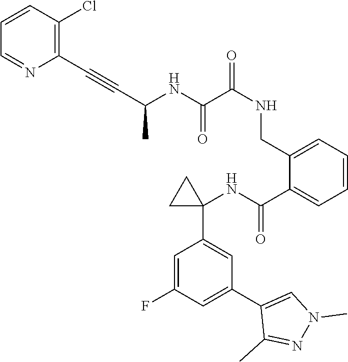 [SMILES: Cc1nn(C)cc1-c1cc(F)cc(C2(NC(=O)c3ccccc3CNC(=O)C(=O)N[C@@H](C)C#Cc3ncccc3Cl)CC2)c1] |
| 276 | 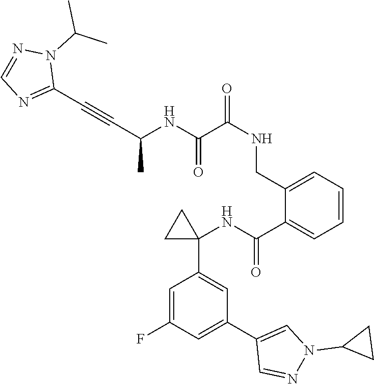 [SMILES: CC(C)n1ncnc1C#C[C@H](C)NC(=O)C(=O)NCc1ccccc1C(=O)NC1(c2cc(F)cc(-c3cnn(C4CC4)c3)c2)CC1] |
| 277 | 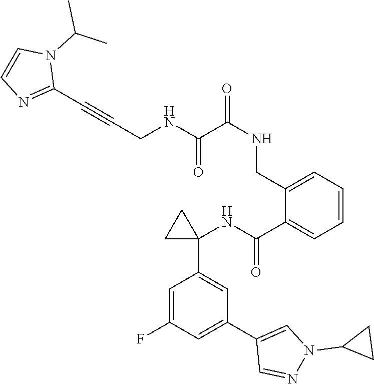 [SMILES: CC(C)n1ccnc1C#CCNC(=O)C(=O)NCc1ccccc1C(=O)NC1(c2cc(F)cc(-c3cnn(C4CC4)c3)c2)CC1] |
| 278 | 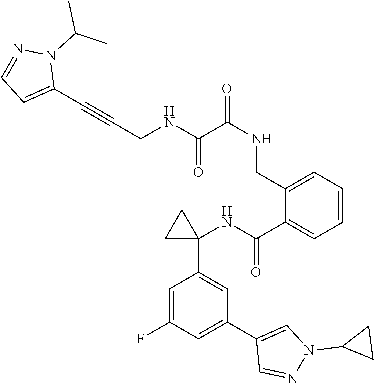 [SMILES: CC(C)n1nccc1C#CCNC(=O)C(=O)NCc1ccccc1C(=O)NC1(c2cc(F)cc(-c3cnn(C4CC4)c3)c2)CC1] |
| 279 | 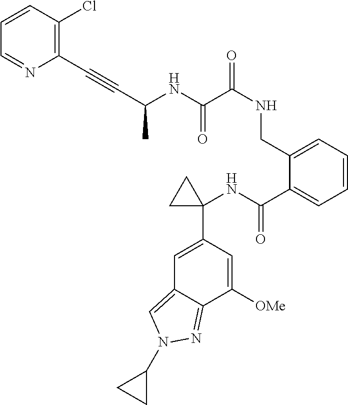 [SMILES: COc1cc(C2(NC(=O)c3ccccc3CNC(=O)C(=O)N[C@@H](C)C#Cc3ncccc3Cl)CC2)cc2cn(C3CC3)nc12] |
| 280 | 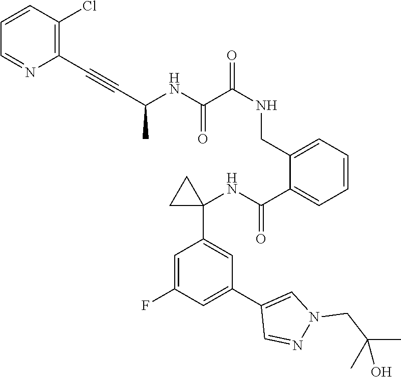 [SMILES: C[C@@H](C#Cc1ncccc1Cl)NC(=O)C(=O)NCc1ccccc1C(=O)NC1(c2cc(F)cc(-c3cnn(CC(C)(C)O)c3)c2)CC1] |
| 281 | 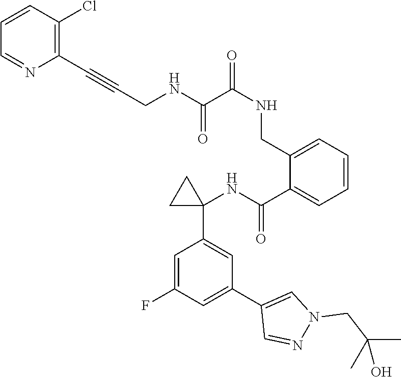 [SMILES: CC(C)(O)Cn1cc(-c2cc(F)cc(C3(NC(=O)c4ccccc4CNC(=O)C(=O)NCC#Cc4ncccc4Cl)CC3)c2)cn1] |
| 282 | 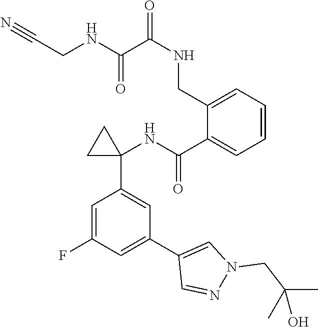 [SMILES: CC(C)(O)Cn1cc(-c2cc(F)cc(C3(NC(=O)c4ccccc4CNC(=O)C(=O)NCC#N)CC3)c2)cn1] |
| 283 | 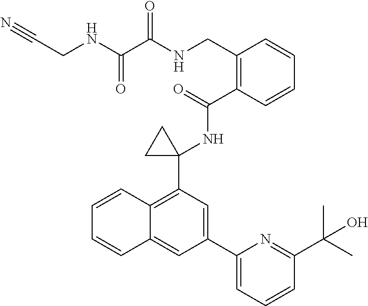 [SMILES: CC(C)(O)c1cccc(-c2cc(C3(NC(=O)c4ccccc4CNC(=O)C(=O)NCC#N)CC3)c3ccccc3c2)n1] |
| 284 | 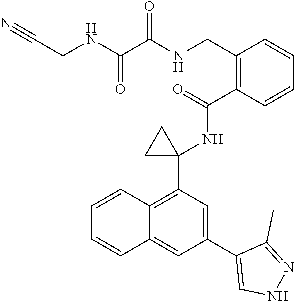 [SMILES: Cc1n[nH]cc1-c1cc(C2(NC(=O)c3ccccc3CNC(=O)C(=O)NCC#N)CC2)c2ccccc2c1] |
| 285 | 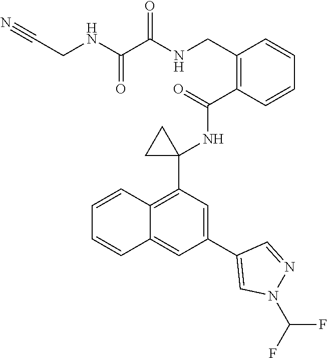 [SMILES: N#CCNC(=O)C(=O)NCc1ccccc1C(=O)NC1(c2cc(-c3cnn(C(F)F)c3)cc3ccccc23)CC1] |
| 286 | 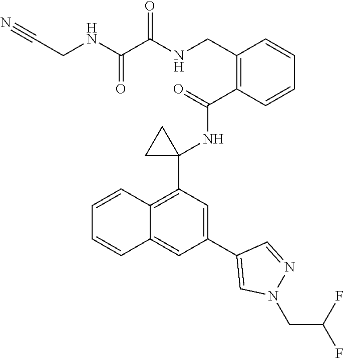 [SMILES: N#CCNC(=O)C(=O)NCc1ccccc1C(=O)NC1(c2cc(-c3cnn(CC(F)F)c3)cc3ccccc23)CC1] |
| 287 | 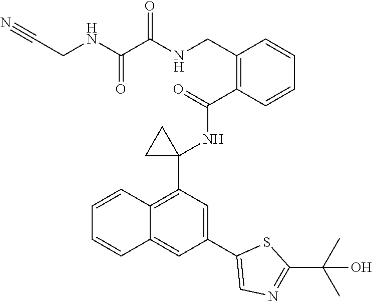 [SMILES: CC(C)(O)c1ncc(-c2cc(C3(NC(=O)c4ccccc4CNC(=O)C(=O)NCC#N)CC3)c3ccccc3c2)s1] |
| 288 | 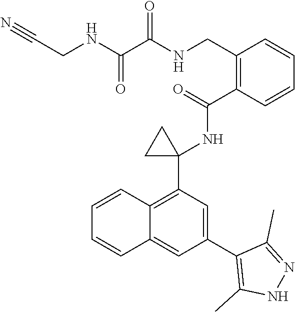 [SMILES: Cc1n[nH]c(C)c1-c1cc(C2(NC(=O)c3ccccc3CNC(=O)C(=O)NCC#N)CC2)c2ccccc2c1] |
| 289 | 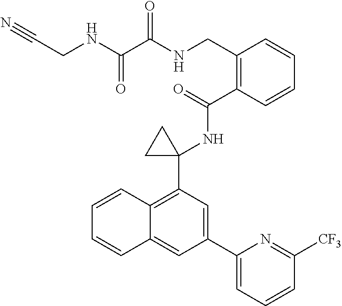 [SMILES: N#CCNC(=O)C(=O)NCc1ccccc1C(=O)NC1(c2cc(-c3cccc(C(F)(F)F)n3)cc3ccccc23)CC1] |
| 290 | 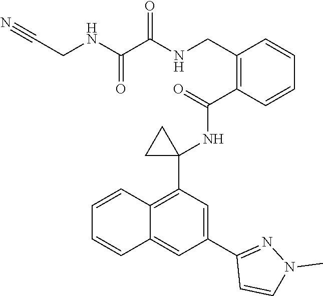 [SMILES: Cn1ccc(-c2cc(C3(NC(=O)c4ccccc4CNC(=O)C(=O)NCC#N)CC3)c3ccccc3c2)n1] |
| 291 | 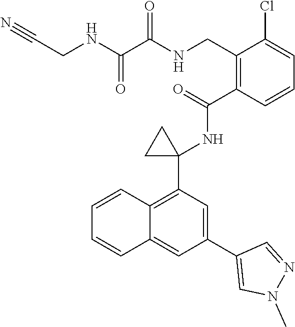 [SMILES: Cn1cc(-c2cc(C3(NC(=O)c4cccc(Cl)c4CNC(=O)C(=O)NCC#N)CC3)c3ccccc3c2)cn1] |
| 292 | 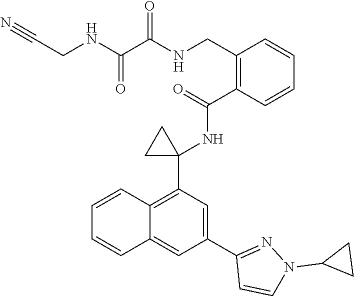 [SMILES: N#CCNC(=O)C(=O)NCc1ccccc1C(=O)NC1(c2cc(-c3ccn(C4CC4)n3)cc3ccccc23)CC1] |
| 293 | 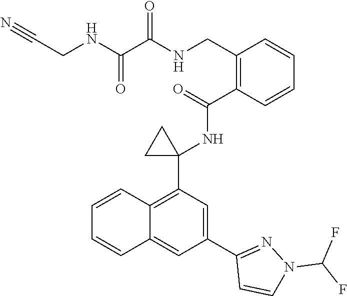 [SMILES: N#CCNC(=O)C(=O)NCc1ccccc1C(=O)NC1(c2cc(-c3ccn(C(F)F)n3)cc3ccccc23)CC1] |
| 294 | 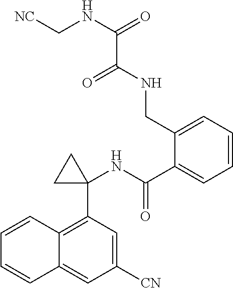 [SMILES: N#CCNC(=O)C(=O)NCc1ccccc1C(=O)NC1(c2cc(C#N)cc3ccccc23)CC1] |
| 295 | 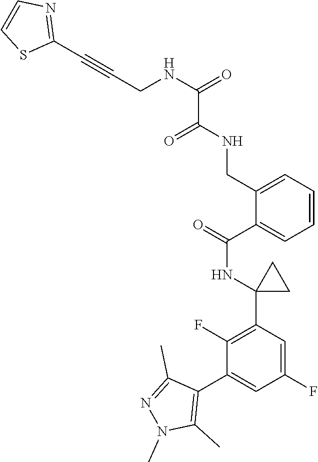 [SMILES: Cc1nn(C)c(C)c1-c1cc(F)cc(C2(NC(=O)c3ccccc3CNC(=O)C(=O)NCC#Cc3nccs3)CC2)c1F] |
| 296 | 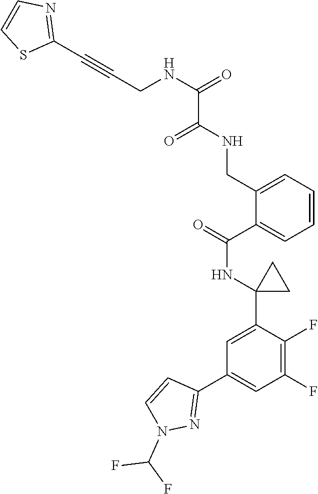 [SMILES: O=C(NCC#Cc1nccs1)C(=O)NCc1ccccc1C(=O)NC1(c2cc(-c3ccn(C(F)F)n3)cc(F)c2F)CC1] |
| 297 | 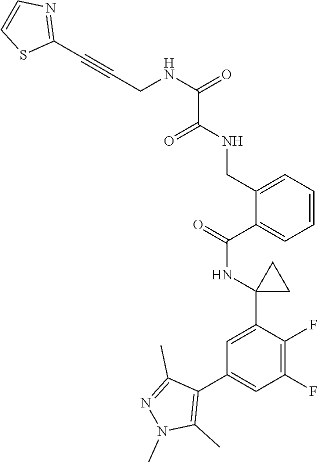 [SMILES: Cc1nn(C)c(C)c1-c1cc(F)c(F)c(C2(NC(=O)c3ccccc3CNC(=O)C(=O)NCC#Cc3nccs3)CC2)c1] |
| 298 | 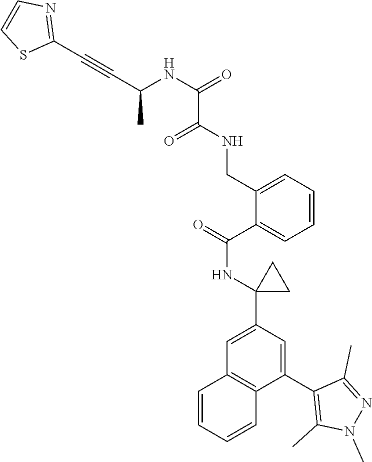 [SMILES: Cc1nn(C)c(C)c1-c1cc(C2(NC(=O)c3ccccc3CNC(=O)C(=O)N[C@@H](C)C#Cc3nccs3)CC2)cc2ccccc12] |
| 299 | 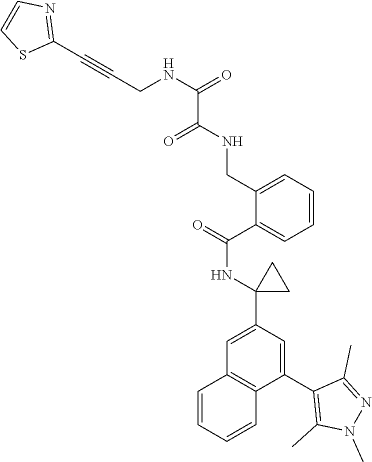 [SMILES: Cc1nn(C)c(C)c1-c1cc(C2(NC(=O)c3ccccc3CNC(=O)C(=O)NCC#Cc3nccs3)CC2)cc2ccccc12] |
| 300 | 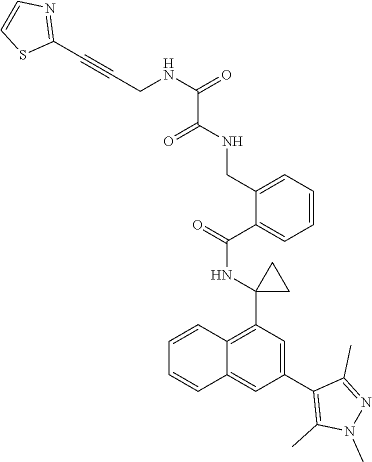 [SMILES: Cc1nn(C)c(C)c1-c1cc(C2(NC(=O)c3ccccc3CNC(=O)C(=O)NCC#Cc3nccs3)CC2)c2ccccc2c1] |
| 301 | 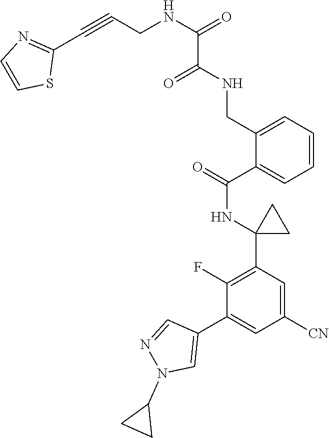 [SMILES: N#Cc1cc(-c2cnn(C3CC3)c2)c(F)c(C2(NC(=O)c3ccccc3CNC(=O)C(=O)NCC#Cc3nccs3)CC2)c1] |
| 302 | 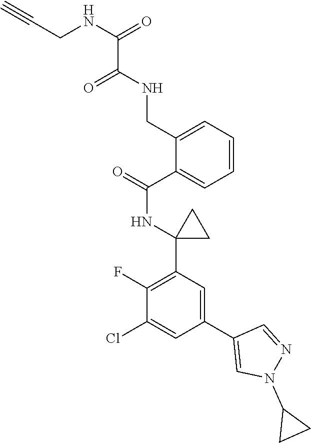 [SMILES: C#CCNC(=O)C(=O)NCc1ccccc1C(=O)NC1(c2cc(-c3cnn(C4CC4)c3)cc(Cl)c2F)CC1] |
| 303 | 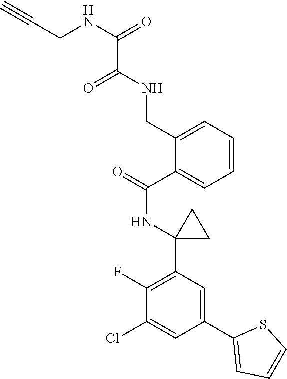 [SMILES: C#CCNC(=O)C(=O)NCc1ccccc1C(=O)NC1(c2cc(-c3cccs3)cc(Cl)c2F)CC1] |
| 304 | 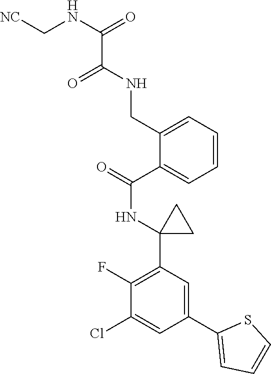 [SMILES: N#CCNC(=O)C(=O)NCc1ccccc1C(=O)NC1(c2cc(-c3cccs3)cc(Cl)c2F)CC1] |
| 305 | 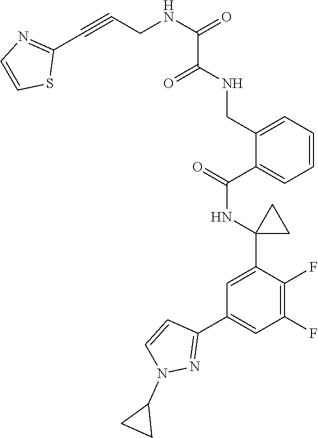 [SMILES: O=C(NCC#Cc1nccs1)C(=O)NCc1ccccc1C(=O)NC1(c2cc(-c3ccn(C4CC4)n3)cc(F)c2F)CC1] |
| 306 | 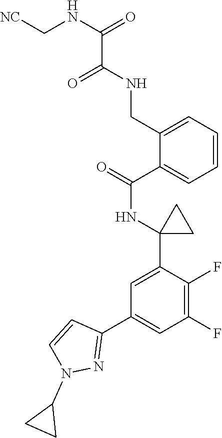 [SMILES: N#CCNC(=O)C(=O)NCc1ccccc1C(=O)NC1(c2cc(-c3ccn(C4CC4)n3)cc(F)c2F)CC1] |
| 307 | 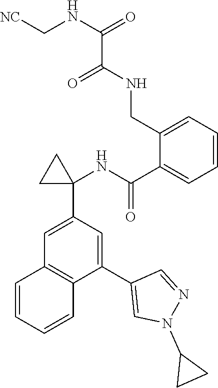 [SMILES: N#CCNC(=O)C(=O)NCc1ccccc1C(=O)NC1(c2cc(-c3cnn(C4CC4)c3)c3ccccc3c2)CC1] |
| 308 | 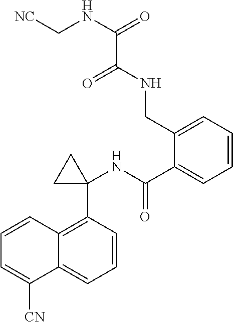 [SMILES: N#CCNC(=O)C(=O)NCc1ccccc1C(=O)NC1(c2cccc3c(C#N)cccc23)CC1] |
| 309 | 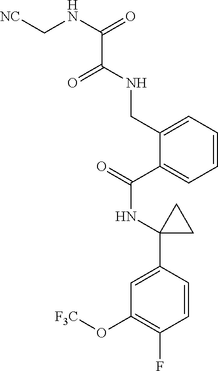 [SMILES: N#CCNC(=O)C(=O)NCc1ccccc1C(=O)NC1(c2ccc(F)c(OC(F)(F)F)c2)CC1] |
| 310 | 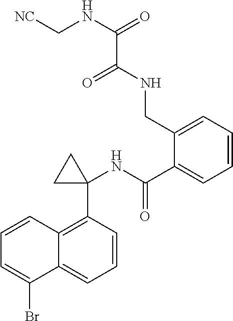 [SMILES: N#CCNC(=O)C(=O)NCc1ccccc1C(=O)NC1(c2cccc3c(Br)cccc23)CC1] |
| 311 | 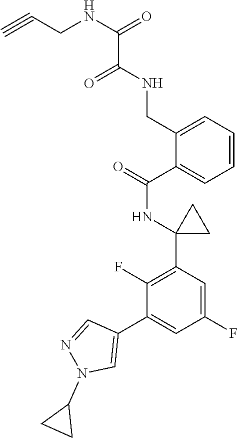 [SMILES: C#CCNC(=O)C(=O)NCc1ccccc1C(=O)NC1(c2cc(F)cc(-c3cnn(C4CC4)c3)c2F)CC1] |
| 312 | 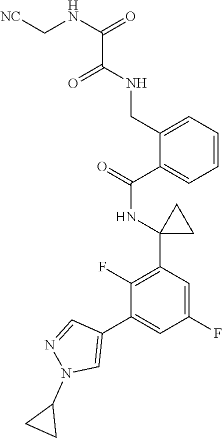 [SMILES: N#CCNC(=O)C(=O)NCc1ccccc1C(=O)NC1(c2cc(F)cc(-c3cnn(C4CC4)c3)c2F)CC1] |
| 313 | 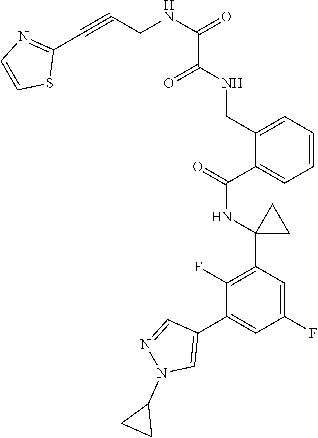 [SMILES: O=C(NCC#Cc1nccs1)C(=O)NCc1ccccc1C(=O)NC1(c2cc(F)cc(-c3cnn(C4CC4)c3)c2F)CC1] |
| 314 | 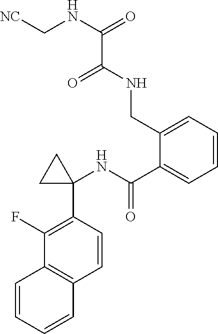 [SMILES: N#CCNC(=O)C(=O)NCc1ccccc1C(=O)NC1(c2ccc3ccccc3c2F)CC1] |
| 315 | 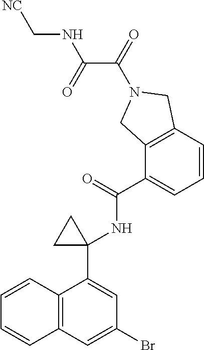 [SMILES: N#CCNC(=O)C(=O)N1Cc2cccc(C(=O)NC3(c4cc(Br)cc5ccccc45)CC3)c2C1] |
| 316 | 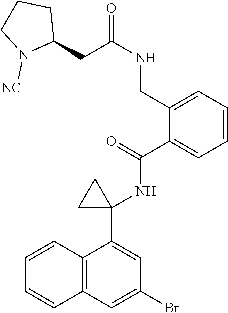 [SMILES: N#CN1CCC[C@H]1CC(=O)NCc1ccccc1C(=O)NC1(c2cc(Br)cc3ccccc23)CC1] |
| 317 | 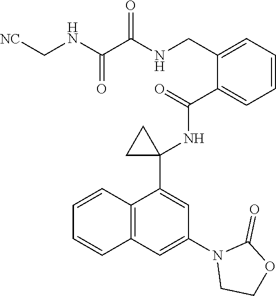 [SMILES: N#CCNC(=O)C(=O)NCc1ccccc1C(=O)NC1(c2cc(N3CCOC3=O)cc3ccccc23)CC1] |
| 318 | 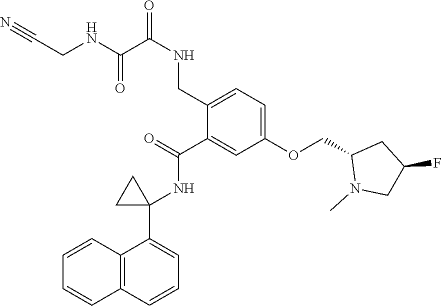 [SMILES: CN1C[C@H](F)C[C@H]1COc1ccc(CNC(=O)C(=O)NCC#N)c(C(=O)NC2(c3cccc4ccccc34)CC2)c1] |
| 319 | 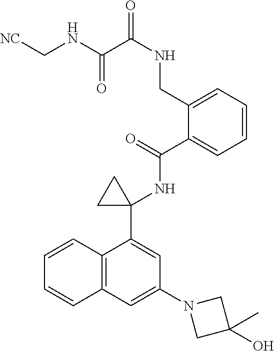 [SMILES: CC1(O)CN(c2cc(C3(NC(=O)c4ccccc4CNC(=O)C(=O)NCC#N)CC3)c3ccccc3c2)C1] |
| 320 | 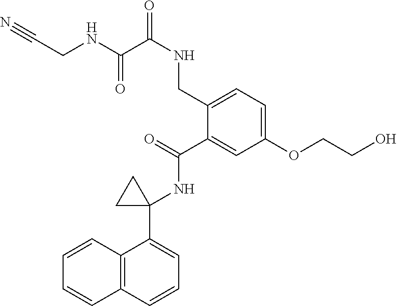 [SMILES: N#CCNC(=O)C(=O)NCc1ccc(OCCO)cc1C(=O)NC1(c2cccc3ccccc23)CC1] |
| 321 | 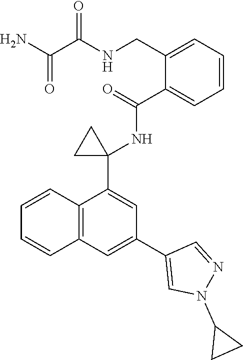 [SMILES: NC(=O)C(=O)NCc1ccccc1C(=O)NC1(c2cc(-c3cnn(C4CC4)c3)cc3ccccc23)CC1] |
| 322 | 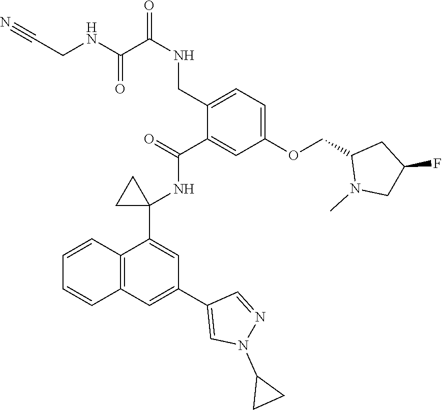 |
| 323 | 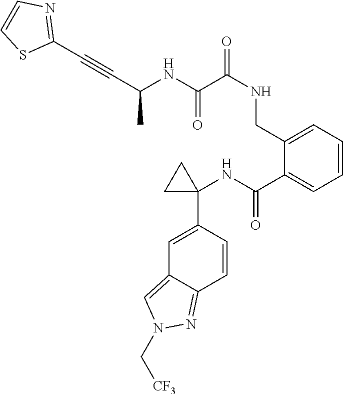 [SMILES: C[C@@H](C#Cc1nccs1)NC(=O)C(=O)NCc1ccccc1C(=O)NC1(c2ccc3nn(CC(F)(F)F)cc3c2)CC1] |
| 324 | 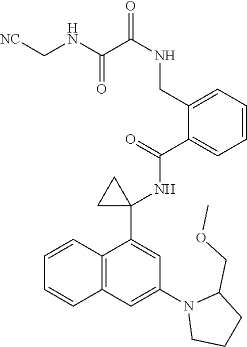 [SMILES: COCC1CCCN1c1cc(C2(NC(=O)c3ccccc3CNC(=O)C(=O)NCC#N)CC2)c2ccccc2c1] |
| 325 | 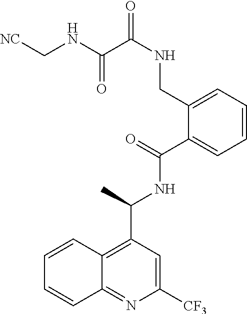 [SMILES: C[C@@H](NC(=O)c1ccccc1CNC(=O)C(=O)NCC#N)c1cc(C(F)(F)F)nc2ccccc12] |
| 326 | 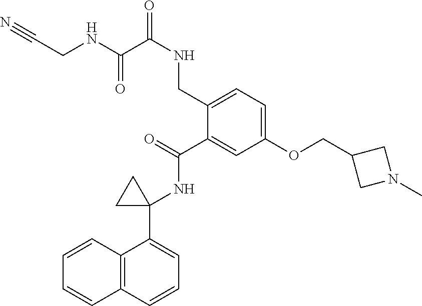 [SMILES: CN1CC(COc2ccc(CNC(=O)C(=O)NCC#N)c(C(=O)NC3(c4cccc5ccccc45)CC3)c2)C1] |
| 327 | 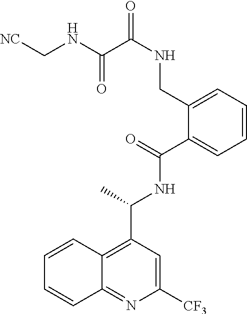 [SMILES: C[C@H](NC(=O)c1ccccc1CNC(=O)C(=O)NCC#N)c1cc(C(F)(F)F)nc2ccccc12] |
| 328 | 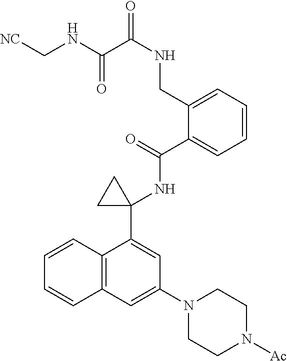 [SMILES: CC(=O)N1CCN(c2cc(C3(NC(=O)c4ccccc4CNC(=O)C(=O)NCC#N)CC3)c3ccccc3c2)CC1] |
| 329 | 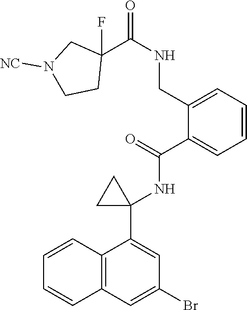 [SMILES: N#CN1CCC(F)(C(=O)NCc2ccccc2C(=O)NC2(c3cc(Br)cc4ccccc34)CC2)C1] |
| 330 | 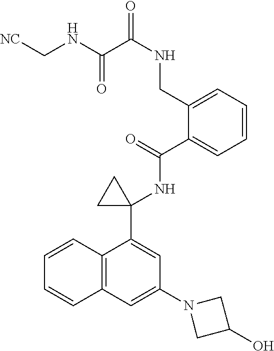 [SMILES: N#CCNC(=O)C(=O)NCc1ccccc1C(=O)NC1(c2cc(N3CC(O)C3)cc3ccccc23)CC1] |
| 331 | 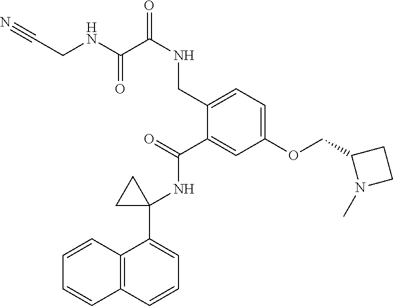 |
| 332 | 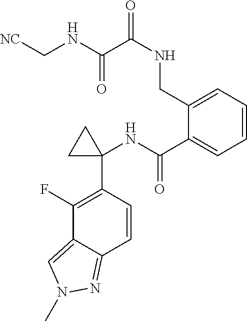 [SMILES: Cn1cc2c(F)c(C3(NC(=O)c4ccccc4CNC(=O)C(=O)NCC#N)CC3)ccc2n1] |
| 333 | 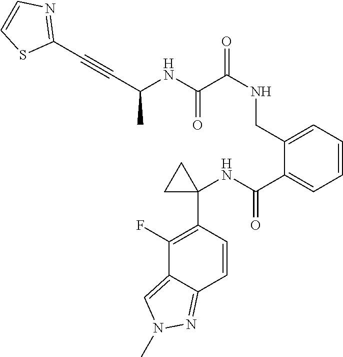 [SMILES: C[C@@H](C#Cc1nccs1)NC(=O)C(=O)NCc1ccccc1C(=O)NC1(c2ccc3nn(C)cc3c2F)CC1] |
| 334 | 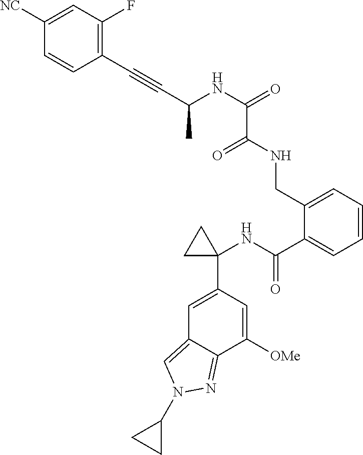 [SMILES: COc1cc(C2(NC(=O)c3ccccc3CNC(=O)C(=O)N[C@@H](C)C#Cc3ccc(C#N)cc3F)CC2)cc2cn(C3CC3)nc12] |
| 335 | 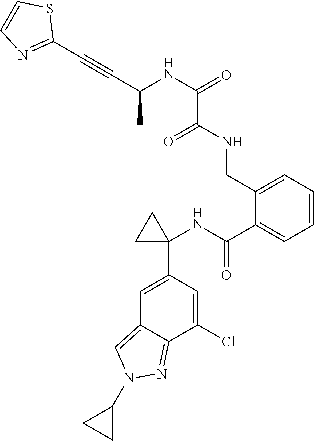 [SMILES: C[C@@H](C#Cc1nccs1)NC(=O)C(=O)NCc1ccccc1C(=O)NC1(c2cc(Cl)c3nn(C4CC4)cc3c2)CC1] |
| 336 | 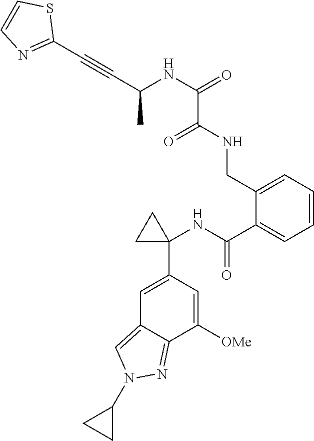 [SMILES: COc1cc(C2(NC(=O)c3ccccc3CNC(=O)C(=O)N[C@@H](C)C#Cc3nccs3)CC2)cc2cn(C3CC3)nc12] |
| 337 | 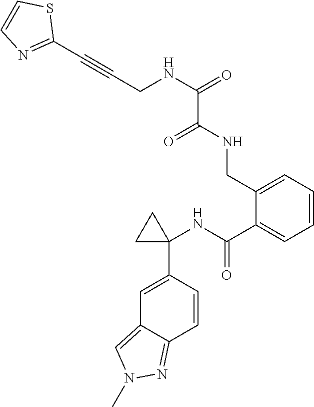 [SMILES: Cn1cc2cc(C3(NC(=O)c4ccccc4CNC(=O)C(=O)NCC#Cc4nccs4)CC3)ccc2n1] |
| 338 | 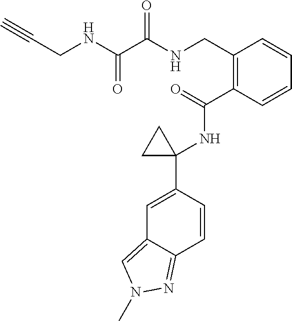 [SMILES: C#CCNC(=O)C(=O)NCc1ccccc1C(=O)NC1(c2ccc3nn(C)cc3c2)CC1] |
| 339 | 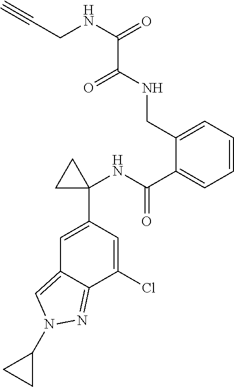 [SMILES: C#CCNC(=O)C(=O)NCc1ccccc1C(=O)NC1(c2cc(Cl)c3nn(C4CC4)cc3c2)CC1] |
| 340 | 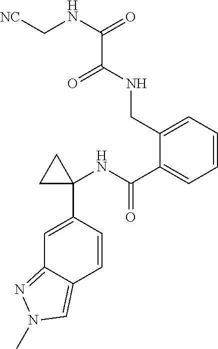 [SMILES: Cn1cc2ccc(C3(NC(=O)c4ccccc4CNC(=O)C(=O)NCC#N)CC3)cc2n1] |
| 341 | 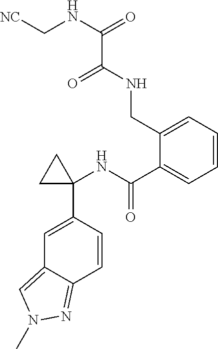 [SMILES: Cn1cc2cc(C3(NC(=O)c4ccccc4CNC(=O)C(=O)NCC#N)CC3)ccc2n1] |
| 342 | 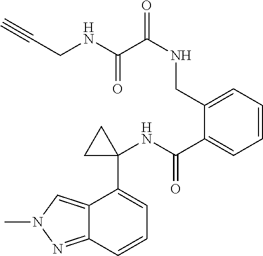 [SMILES: C#CCNC(=O)C(=O)NCc1ccccc1C(=O)NC1(c2cccc3nn(C)cc23)CC1] |
| 343 | 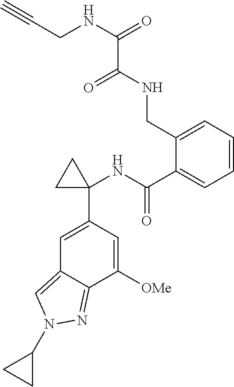 [SMILES: C#CCNC(=O)C(=O)NCc1ccccc1C(=O)NC1(c2cc(OC)c3nn(C4CC4)cc3c2)CC1] |
| 344 | 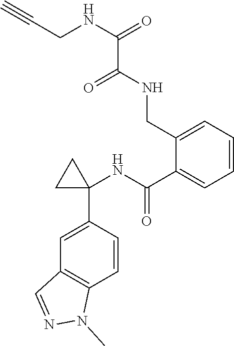 [SMILES: C#CCNC(=O)C(=O)NCc1ccccc1C(=O)NC1(c2ccc3c(cnn3C)c2)CC1] |
| 345 | 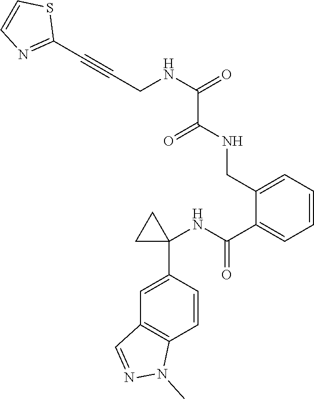 [SMILES: Cn1ncc2cc(C3(NC(=O)c4ccccc4CNC(=O)C(=O)NCC#Cc4nccs4)CC3)ccc21] |
| 346 | 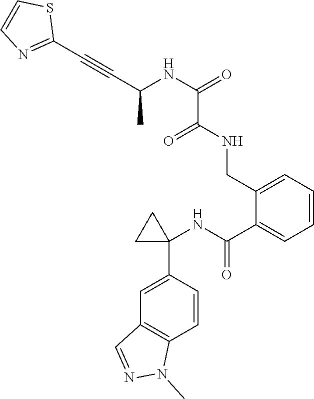 [SMILES: C[C@@H](C#Cc1nccs1)NC(=O)C(=O)NCc1ccccc1C(=O)NC1(c2ccc3c(cnn3C)c2)CC1] |
| 347 | 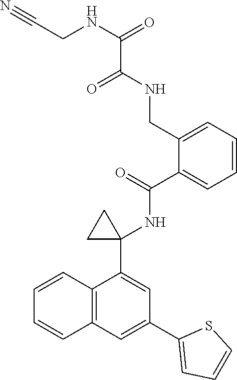 [SMILES: N#CCNC(=O)C(=O)NCc1ccccc1C(=O)NC1(c2cc(-c3cccs3)cc3ccccc23)CC1] |
| 348 | 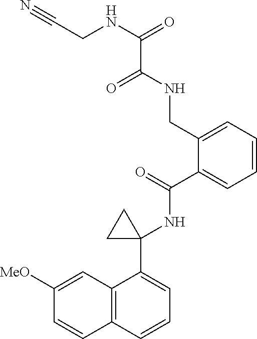 [SMILES: COc1ccc2cccc(C3(NC(=O)c4ccccc4CNC(=O)C(=O)NCC#N)CC3)c2c1] |
| 349 | 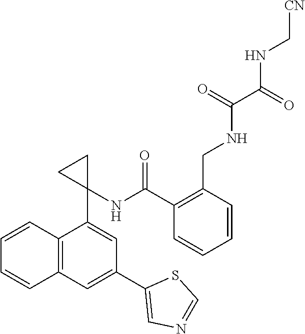 [SMILES: N#CCNC(=O)C(=O)NCc1ccccc1C(=O)NC1(c2cc(-c3cncs3)cc3ccccc23)CC1] |
| 350 | 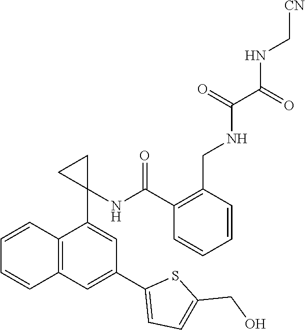 [SMILES: N#CCNC(=O)C(=O)NCc1ccccc1C(=O)NC1(c2cc(-c3ccc(CO)s3)cc3ccccc23)CC1] |
| 351 | 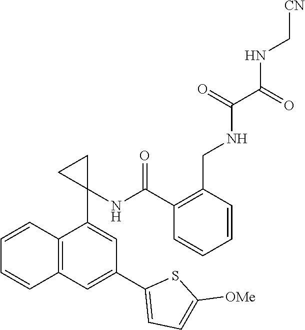 [SMILES: COc1ccc(-c2cc(C3(NC(=O)c4ccccc4CNC(=O)C(=O)NCC#N)CC3)c3ccccc3c2)s1] |
| 352 | 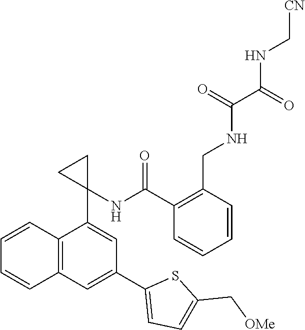 [SMILES: COCc1ccc(-c2cc(C3(NC(=O)c4ccccc4CNC(=O)C(=O)NCC#N)CC3)c3ccccc3c2)s1] |
| 353 | 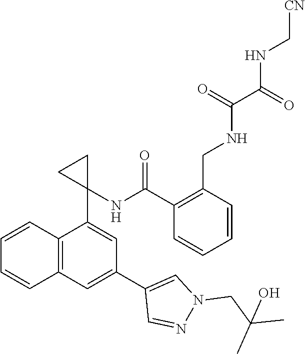 [SMILES: CC(C)(O)Cn1cc(-c2cc(C3(NC(=O)c4ccccc4CNC(=O)C(=O)NCC#N)CC3)c3ccccc3c2)cn1] |
| 354 | 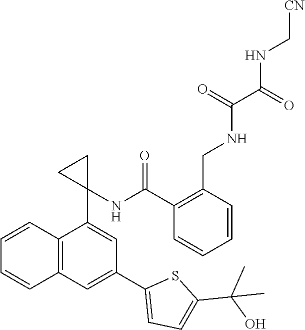 [SMILES: CC(C)(O)c1ccc(-c2cc(C3(NC(=O)c4ccccc4CNC(=O)C(=O)NCC#N)CC3)c3ccccc3c2)s1] |
| 355 | 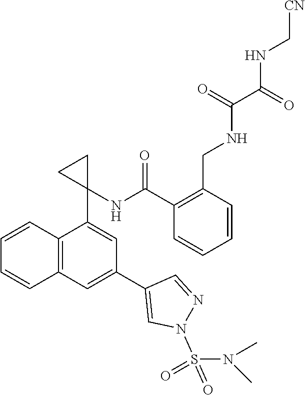 [SMILES: CN(C)S(=O)(=O)n1cc(-c2cc(C3(NC(=O)c4ccccc4CNC(=O)C(=O)NCC#N)CC3)c3ccccc3c2)cn1] |
| 356 | 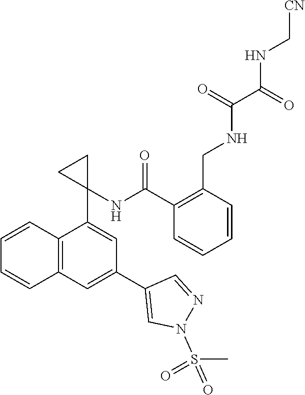 [SMILES: CS(=O)(=O)n1cc(-c2cc(C3(NC(=O)c4ccccc4CNC(=O)C(=O)NCC#N)CC3)c3ccccc3c2)cn1] |
| 357 | 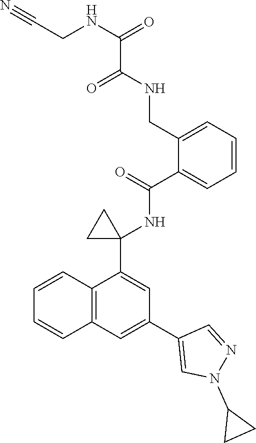 [SMILES: N#CCNC(=O)C(=O)NCc1ccccc1C(=O)NC1(c2cc(-c3cnn(C4CC4)c3)cc3ccccc23)CC1] |
| 358 | 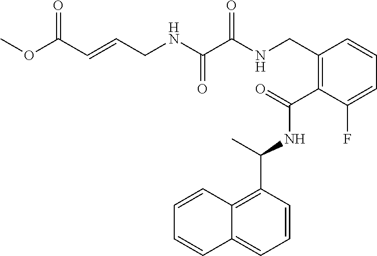 [SMILES: COC(=O)/C=C/CNC(=O)C(=O)NCc1cccc(F)c1C(=O)N[C@H](C)c1cccc2ccccc12] |
| 359 | 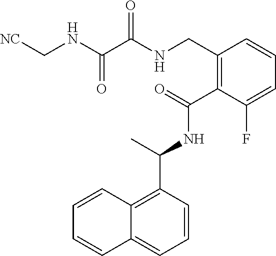 [SMILES: C[C@@H](NC(=O)c1c(F)cccc1CNC(=O)C(=O)NCC#N)c1cccc2ccccc12] |
| 360 | 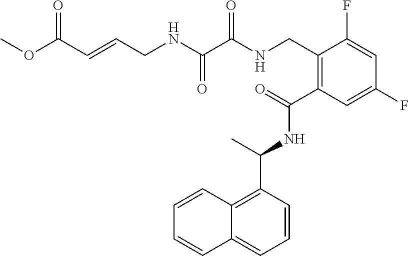 [SMILES: COC(=O)/C=C/CNC(=O)C(=O)NCc1c(F)cc(F)cc1C(=O)N[C@H](C)c1cccc2ccccc12] |
| 361 | 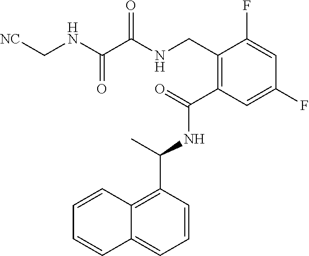 [SMILES: C[C@@H](NC(=O)c1cc(F)cc(F)c1CNC(=O)C(=O)NCC#N)c1cccc2ccccc12] |
| 362 | 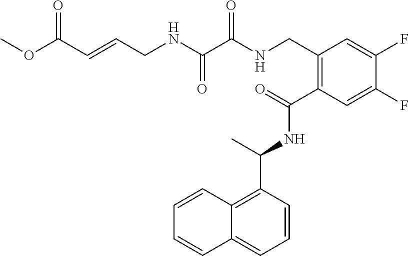 [SMILES: COC(=O)/C=C/CNC(=O)C(=O)NCc1cc(F)c(F)cc1C(=O)N[C@H](C)c1cccc2ccccc12] |
| 363 | 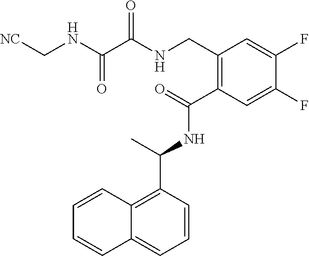 [SMILES: C[C@@H](NC(=O)c1cc(F)c(F)cc1CNC(=O)C(=O)NCC#N)c1cccc2ccccc12] |
| 364 | 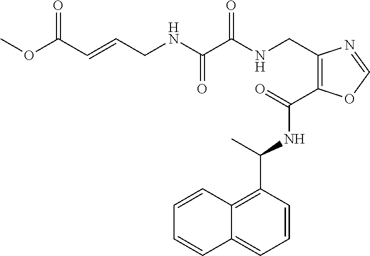 [SMILES: COC(=O)/C=C/CNC(=O)C(=O)NCc1ncoc1C(=O)N[C@H](C)c1cccc2ccccc12] |
| 365 | 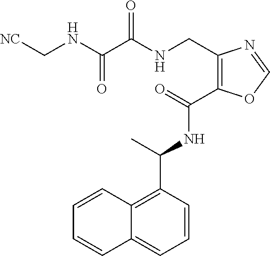 [SMILES: C[C@@H](NC(=O)c1ocnc1CNC(=O)C(=O)NCC#N)c1cccc2ccccc12] |
| 366 | 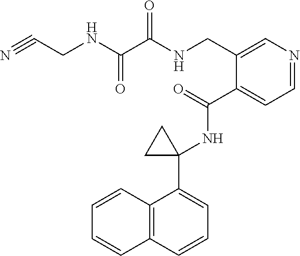 [SMILES: N#CCNC(=O)C(=O)NCc1cnccc1C(=O)NC1(c2cccc3ccccc23)CC1] |
| 367 | 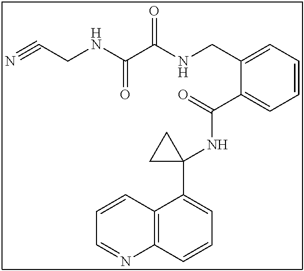 [SMILES: N#CCNC(=O)C(=O)NCc1ccccc1C(=O)NC1(c2cccc3ncccc23)CC1] |
| 368 | 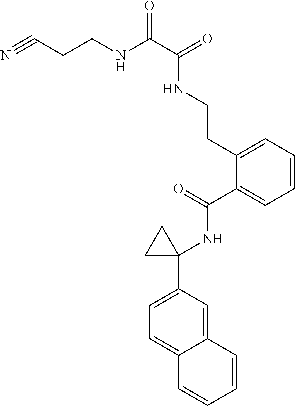 [SMILES: N#CCCNC(=O)C(=O)NCCc1ccccc1C(=O)NC1(c2ccc3ccccc3c2)CC1] |
| 369 | 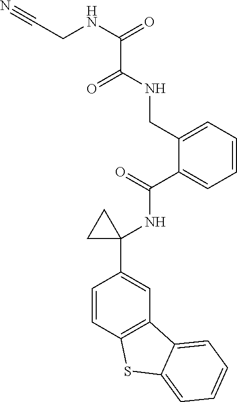 [SMILES: N#CCNC(=O)C(=O)NCc1ccccc1C(=O)NC1(c2ccc3sc4ccccc4c3c2)CC1] |
| 370 | 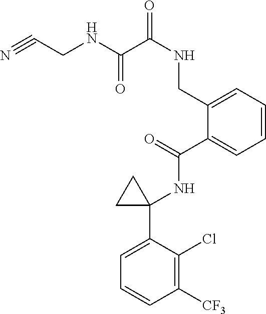 [SMILES: N#CCNC(=O)C(=O)NCc1ccccc1C(=O)NC1(c2cccc(C(F)(F)F)c2Cl)CC1] |
| 371 | 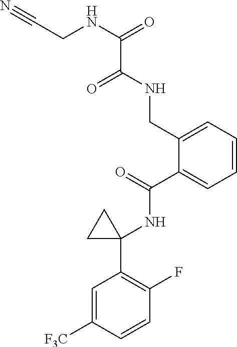 [SMILES: N#CCNC(=O)C(=O)NCc1ccccc1C(=O)NC1(c2cc(C(F)(F)F)ccc2F)CC1] |
| 372 | 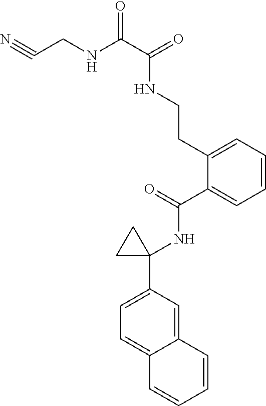 [SMILES: N#CCNC(=O)C(=O)NCCc1ccccc1C(=O)NC1(c2ccc3ccccc3c2)CC1] |
| 373 | 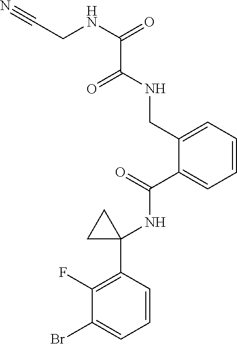 [SMILES: N#CCNC(=O)C(=O)NCc1ccccc1C(=O)NC1(c2cccc(Br)c2F)CC1] |
| 374 | 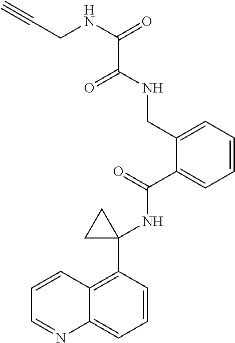 [SMILES: C#CCNC(=O)C(=O)NCc1ccccc1C(=O)NC1(c2cccc3ncccc23)CC1] |
| 375 | 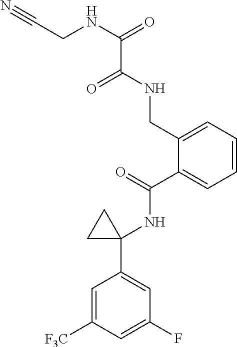 [SMILES: N#CCNC(=O)C(=O)NCc1ccccc1C(=O)NC1(c2cc(F)cc(C(F)(F)F)c2)CC1] |
| 376 | 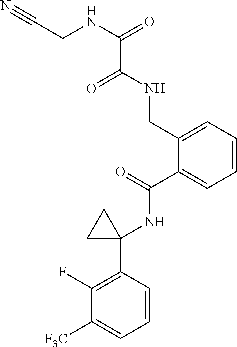 [SMILES: N#CCNC(=O)C(=O)NCc1ccccc1C(=O)NC1(c2cccc(C(F)(F)F)c2F)CC1] |
| 377 | 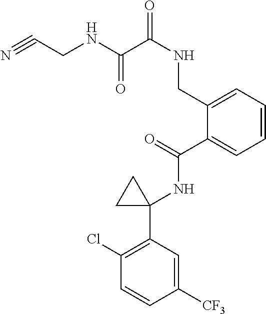 [SMILES: N#CCNC(=O)C(=O)NCc1ccccc1C(=O)NC1(c2cc(C(F)(F)F)ccc2Cl)CC1] |
| 378 | 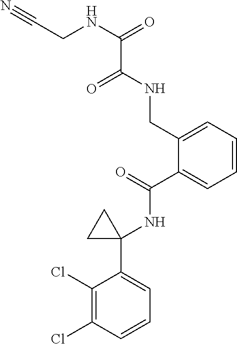 [SMILES: N#CCNC(=O)C(=O)NCc1ccccc1C(=O)NC1(c2cccc(Cl)c2Cl)CC1] |
| 379 | 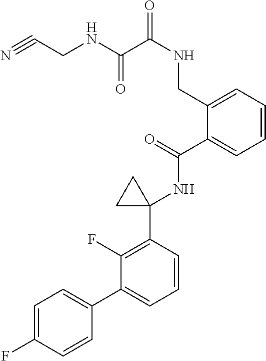 [SMILES: N#CCNC(=O)C(=O)NCc1ccccc1C(=O)NC1(c2cccc(-c3ccc(F)cc3)c2F)CC1] |
| 380 | 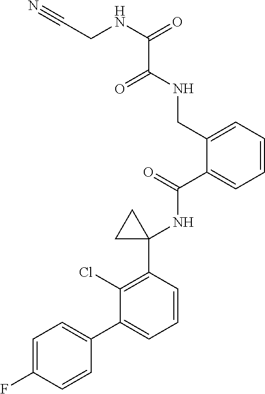 [SMILES: N#CCNC(=O)C(=O)NCc1ccccc1C(=O)NC1(c2cccc(-c3ccc(F)cc3)c2Cl)CC1] |
| 381 | 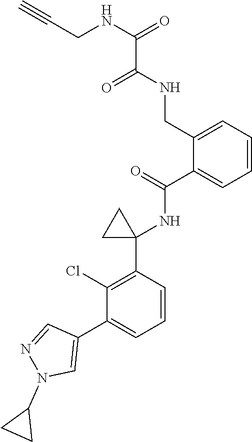 [SMILES: C#CCNC(=O)C(=O)NCc1ccccc1C(=O)NC1(c2cccc(-c3cnn(C4CC4)c3)c2Cl)CC1] |
| 382 | 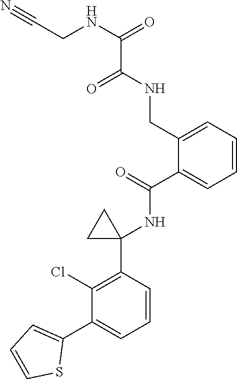 [SMILES: N#CCNC(=O)C(=O)NCc1ccccc1C(=O)NC1(c2cccc(-c3cccs3)c2Cl)CC1] |
| 383 | 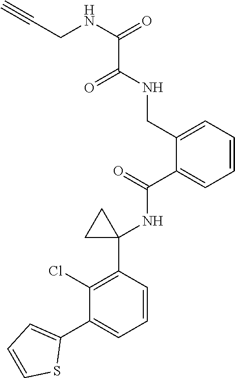 [SMILES: C#CCNC(=O)C(=O)NCc1ccccc1C(=O)NC1(c2cccc(-c3cccs3)c2Cl)CC1] |
| 384 | 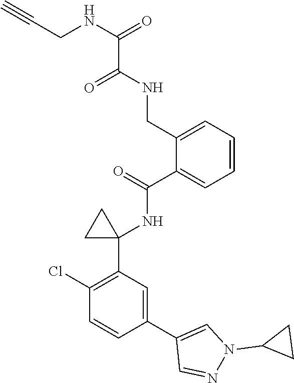 [SMILES: C#CCNC(=O)C(=O)NCc1ccccc1C(=O)NC1(c2cc(-c3cnn(C4CC4)c3)ccc2Cl)CC1] |
| 385 | 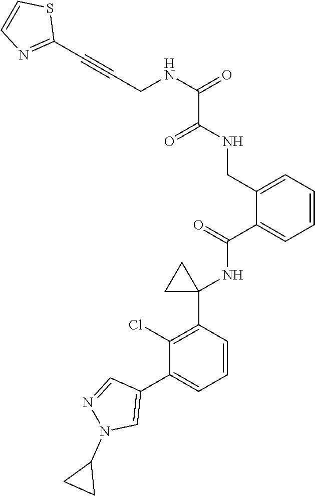 [SMILES: O=C(NCC#Cc1nccs1)C(=O)NCc1ccccc1C(=O)NC1(c2cccc(-c3cnn(C4CC4)c3)c2Cl)CC1] |
| 386 | 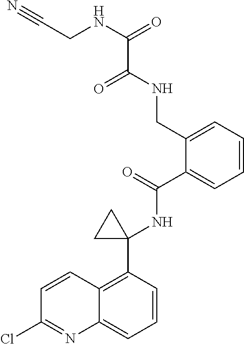 [SMILES: N#CCNC(=O)C(=O)NCc1ccccc1C(=O)NC1(c2cccc3nc(Cl)ccc23)CC1] |
| 387 | 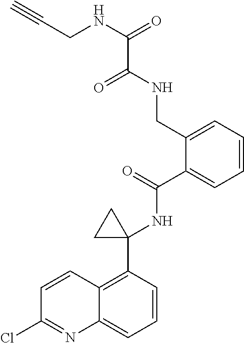 [SMILES: C#CCNC(=O)C(=O)NCc1ccccc1C(=O)NC1(c2cccc3nc(Cl)ccc23)CC1] |
| 388 | 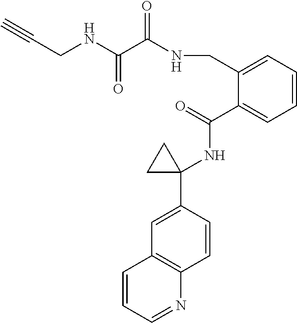 [SMILES: C#CCNC(=O)C(=O)NCc1ccccc1C(=O)NC1(c2ccc3ncccc3c2)CC1] |
| 389 | 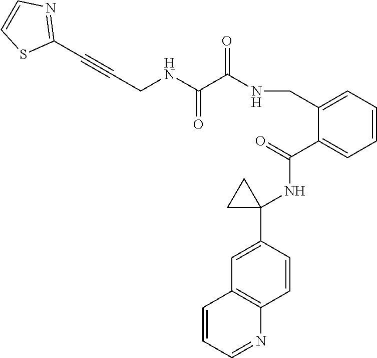 [SMILES: O=C(NCC#Cc1nccs1)C(=O)NCc1ccccc1C(=O)NC1(c2ccc3ncccc3c2)CC1] |
| 390 | 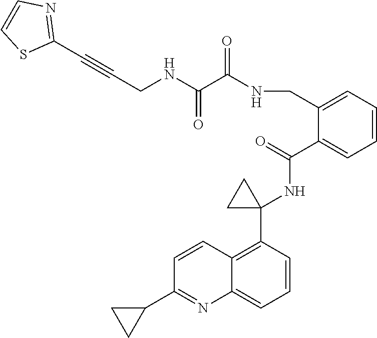 [SMILES: O=C(NCC#Cc1nccs1)C(=O)NCc1ccccc1C(=O)NC1(c2cccc3nc(C4CC4)ccc23)CC1] |
| 391 | 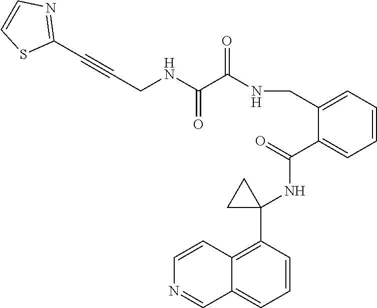 [SMILES: O=C(NCC#Cc1nccs1)C(=O)NCc1ccccc1C(=O)NC1(c2cccc3cnccc23)CC1] |
| 392 | 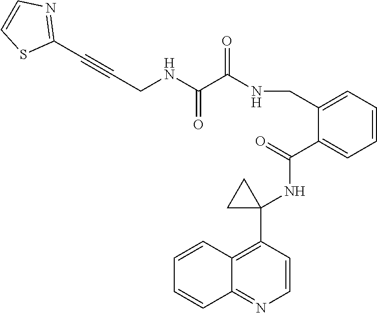 [SMILES: O=C(NCC#Cc1nccs1)C(=O)NCc1ccccc1C(=O)NC1(c2ccnc3ccccc23)CC1] |
| 393 | 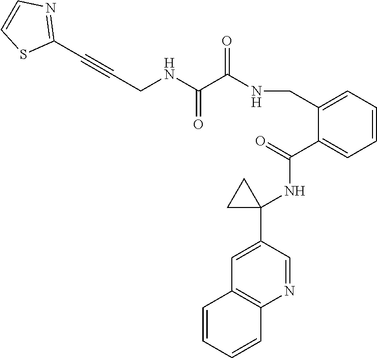 [SMILES: O=C(NCC#Cc1nccs1)C(=O)NCc1ccccc1C(=O)NC1(c2cnc3ccccc3c2)CC1] |
| 394 | 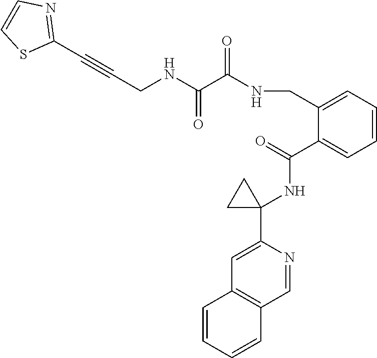 [SMILES: O=C(NCC#Cc1nccs1)C(=O)NCc1ccccc1C(=O)NC1(c2cc3ccccc3cn2)CC1] |
| 395 | 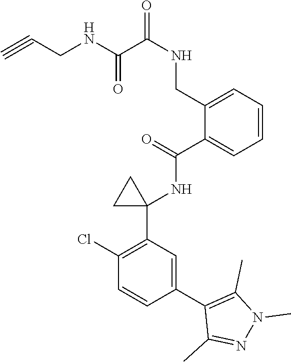 [SMILES: C#CCNC(=O)C(=O)NCc1ccccc1C(=O)NC1(c2cc(-c3c(C)nn(C)c3C)ccc2Cl)CC1] |
| 396 | 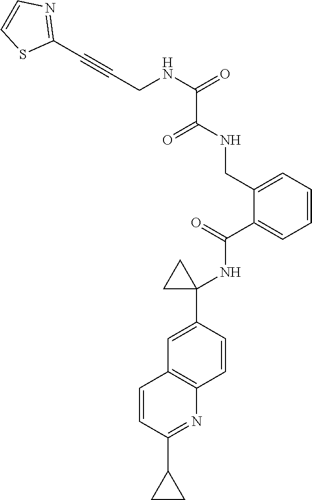 [SMILES: O=C(NCC#Cc1nccs1)C(=O)NCc1ccccc1C(=O)NC1(c2ccc3nc(C4CC4)ccc3c2)CC1] |
| 397 | 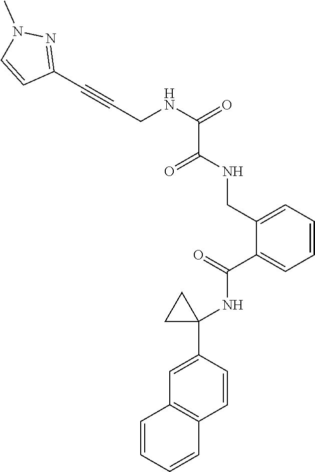 [SMILES: Cn1ccc(C#CCNC(=O)C(=O)NCc2ccccc2C(=O)NC2(c3ccc4ccccc4c3)CC2)n1] |
| 398 | 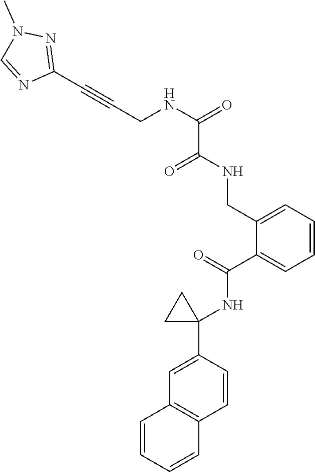 [SMILES: Cn1cnc(C#CCNC(=O)C(=O)NCc2ccccc2C(=O)NC2(c3ccc4ccccc4c3)CC2)n1] |
| 399 | 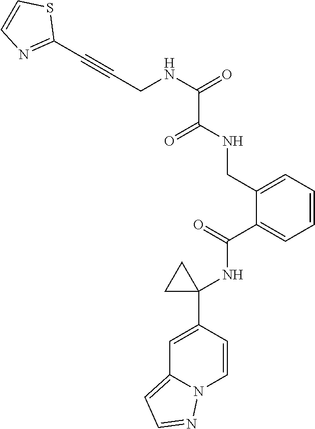 [SMILES: O=C(NCC#Cc1nccs1)C(=O)NCc1ccccc1C(=O)NC1(c2ccn3nccc3c2)CC1] |
| 400 | 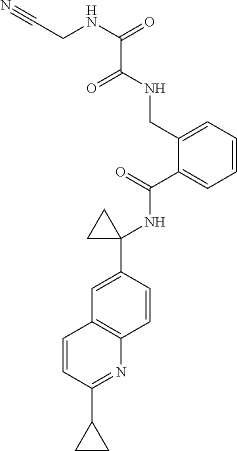 [SMILES: N#CCNC(=O)C(=O)NCc1ccccc1C(=O)NC1(c2ccc3nc(C4CC4)ccc3c2)CC1] |
| 401 | 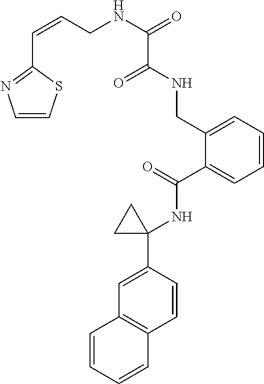 [SMILES: O=C(NC/C=C\c1nccs1)C(=O)NCc1ccccc1C(=O)NC1(c2ccc3ccccc3c2)CC1] |
| 402 | 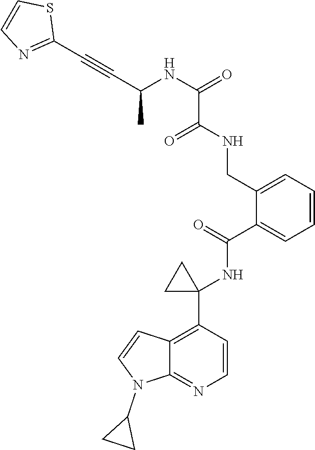 [SMILES: C[C@@H](C#Cc1nccs1)NC(=O)C(=O)NCc1ccccc1C(=O)NC1(c2ccnc3c2ccn3C2CC2)CC1] |
| 403 | 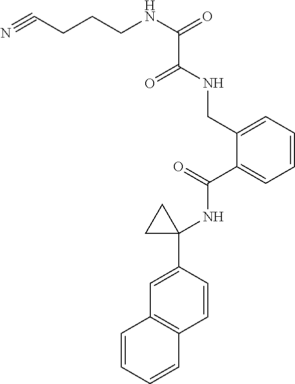 [SMILES: N#CCCCNC(=O)C(=O)NCc1ccccc1C(=O)NC1(c2ccc3ccccc3c2)CC1] |
| 404 | 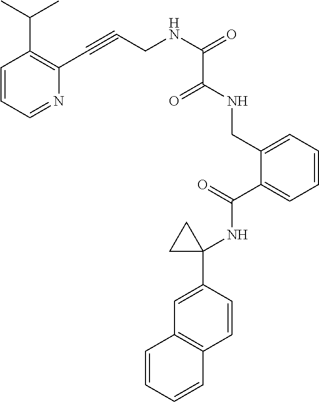 [SMILES: CC(C)c1cccnc1C#CCNC(=O)C(=O)NCc1ccccc1C(=O)NC1(c2ccc3ccccc3c2)CC1] |
| 405 | 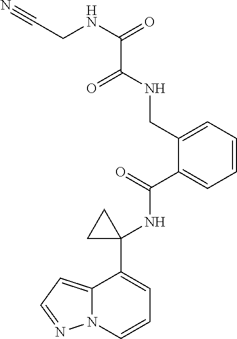 [SMILES: N#CCNC(=O)C(=O)NCc1ccccc1C(=O)NC1(c2cccn3nccc23)CC1] |
| 406 | 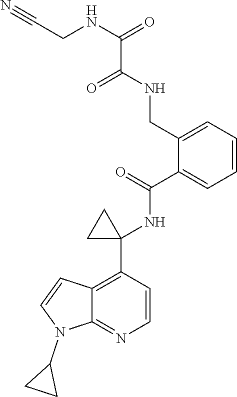 [SMILES: N#CCNC(=O)C(=O)NCc1ccccc1C(=O)NC1(c2ccnc3c2ccn3C2CC2)CC1] |
| 407 | 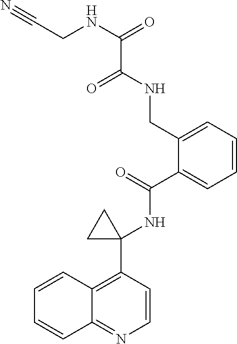 [SMILES: N#CCNC(=O)C(=O)NCc1ccccc1C(=O)NC1(c2ccnc3ccccc23)CC1] |
| 408 | 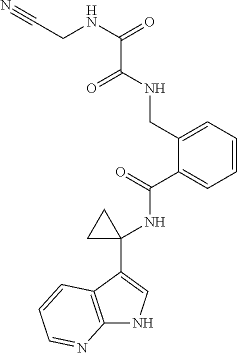 [SMILES: N#CCNC(=O)C(=O)NCc1ccccc1C(=O)NC1(c2c[nH]c3ncccc23)CC1] |
| 409 | 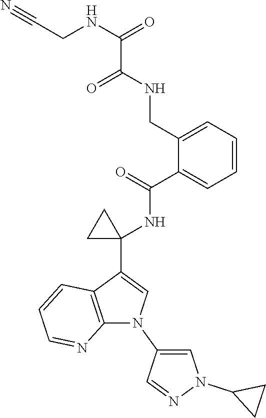 [SMILES: N#CCNC(=O)C(=O)NCc1ccccc1C(=O)NC1(c2cn(-c3cnn(C4CC4)c3)c3ncccc23)CC1] |
| 410 | 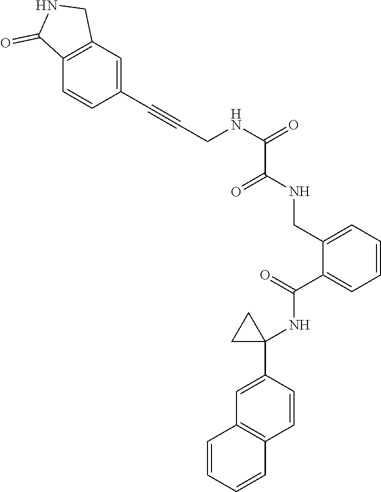 [SMILES: O=C(NCC#Cc1ccc2c(c1)CNC2=O)C(=O)NCc1ccccc1C(=O)NC1(c2ccc3ccccc3c2)CC1] |
| 411 | 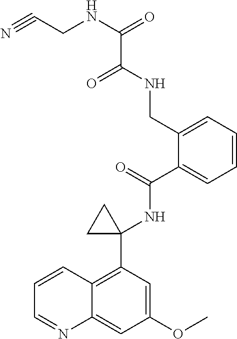 [SMILES: COc1cc(C2(NC(=O)c3ccccc3CNC(=O)C(=O)NCC#N)CC2)c2cccnc2c1] |
| 412 | 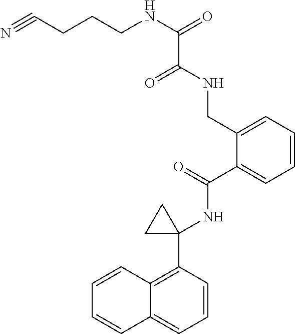 [SMILES: N#CCCCNC(=O)C(=O)NCc1ccccc1C(=O)NC1(c2cccc3ccccc23)CC1] |
| 413 | 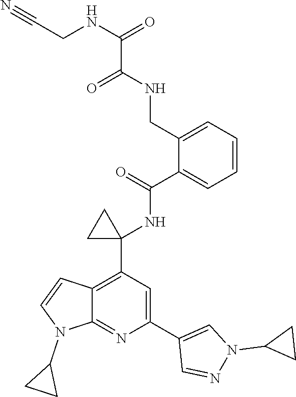 [SMILES: N#CCNC(=O)C(=O)NCc1ccccc1C(=O)NC1(c2cc(-c3cnn(C4CC4)c3)nc3c2ccn3C2CC2)CC1] |
| 414 | 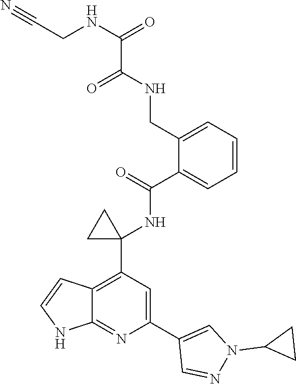 [SMILES: N#CCNC(=O)C(=O)NCc1ccccc1C(=O)NC1(c2cc(-c3cnn(C4CC4)c3)nc3[nH]ccc23)CC1] |
| 415 | 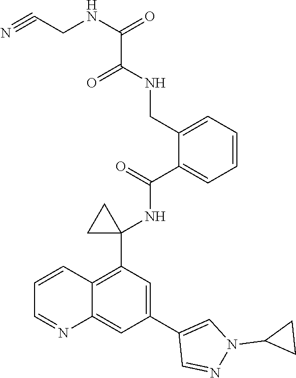 [SMILES: N#CCNC(=O)C(=O)NCc1ccccc1C(=O)NC1(c2cc(-c3cnn(C4CC4)c3)cc3ncccc23)CC1] |
| 416 | 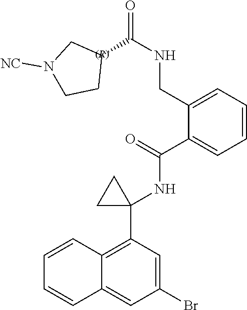 |
| 417 | 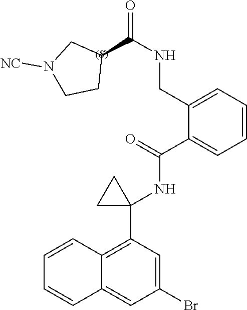 |
| 418 | 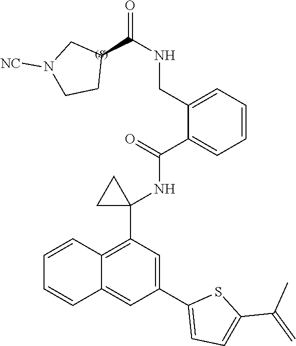 |
| 419 | 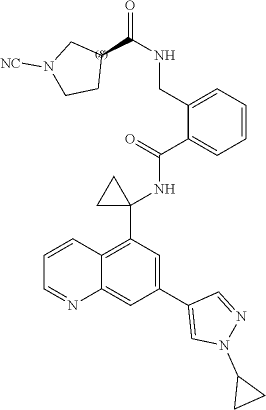 |
| 420 | 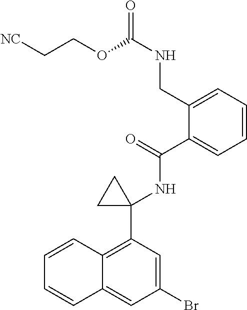 |
| 421 | 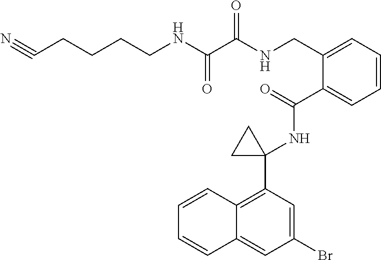 [SMILES: N#CCCCCNC(=O)C(=O)NCc1ccccc1C(=O)NC1(c2cc(Br)cc3ccccc23)CC1] |
| 422 | 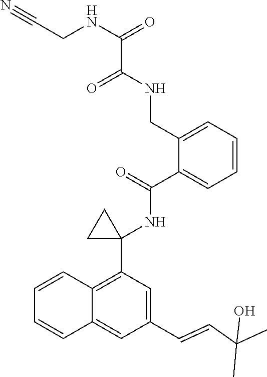 [SMILES: CC(C)(O)/C=C/c1cc(C2(NC(=O)c3ccccc3CNC(=O)C(=O)NCC#N)CC2)c2ccccc2c1] |
| 423 | 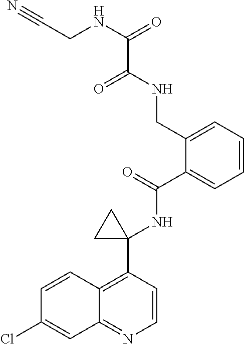 [SMILES: N#CCNC(=O)C(=O)NCc1ccccc1C(=O)NC1(c2ccnc3cc(Cl)ccc23)CC1] |
| 424 | 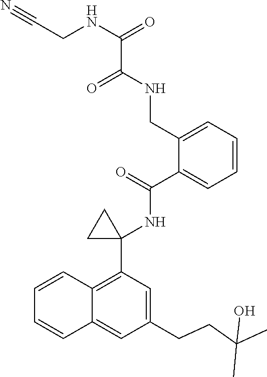 [SMILES: CC(C)(O)CCc1cc(C2(NC(=O)c3ccccc3CNC(=O)C(=O)NCC#N)CC2)c2ccccc2c1] |
| 425 | 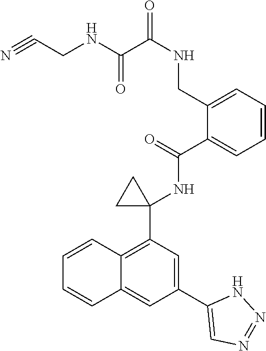 [SMILES: N#CCNC(=O)C(=O)NCc1ccccc1C(=O)NC1(c2cc(-c3cnn[nH]3)cc3ccccc23)CC1] |
| 426 | 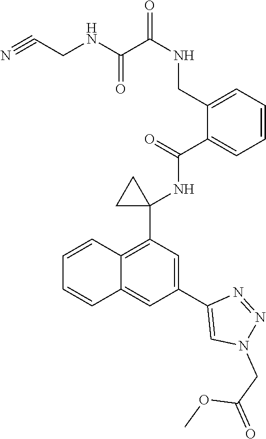 [SMILES: COC(=O)Cn1cc(-c2cc(C3(NC(=O)c4ccccc4CNC(=O)C(=O)NCC#N)CC3)c3ccccc3c2)nn1] |
| 427 | 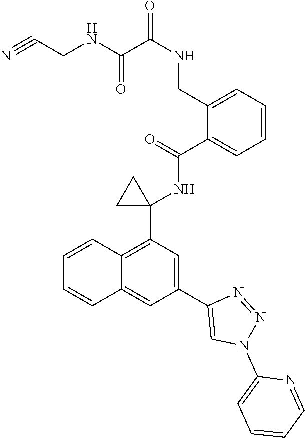 [SMILES: N#CCNC(=O)C(=O)NCc1ccccc1C(=O)NC1(c2cc(-c3cn(-c4ccccn4)nn3)cc3ccccc23)CC1] |
| 428 | 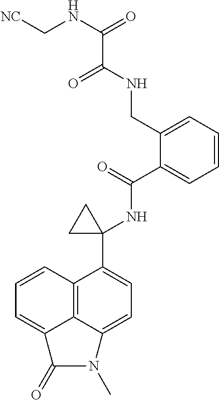 [SMILES: CN1C(=O)c2cccc3c(C4(NC(=O)c5ccccc5CNC(=O)C(=O)NCC#N)CC4)ccc1c23] |
| 429 | 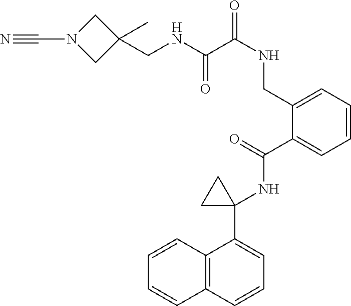 [SMILES: CC1(CNC(=O)C(=O)NCc2ccccc2C(=O)NC2(c3cccc4ccccc34)CC2)CN(C#N)C1] |
| 430 | 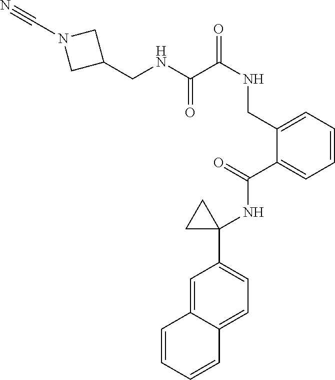 [SMILES: N#CN1CC(CNC(=O)C(=O)NCc2ccccc2C(=O)NC2(c3ccc4ccccc4c3)CC2)C1] |
| 431 | 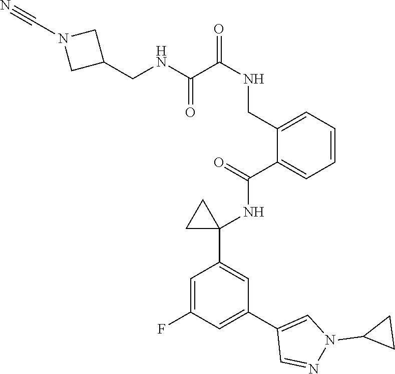 [SMILES: N#CN1CC(CNC(=O)C(=O)NCc2ccccc2C(=O)NC2(c3cc(F)cc(-c4cnn(C5CC5)c4)c3)CC2)C1] |
| 432 | 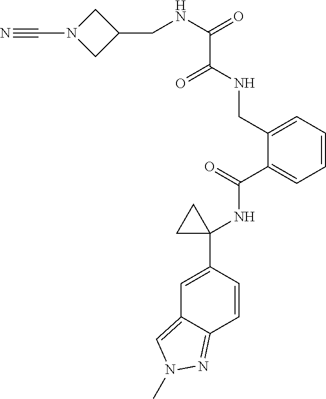 [SMILES: Cn1cc2cc(C3(NC(=O)c4ccccc4CNC(=O)C(=O)NCC4CN(C#N)C4)CC3)ccc2n1] |
| 433 | 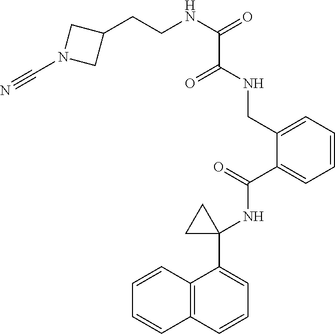 [SMILES: N#CN1CC(CCNC(=O)C(=O)NCc2ccccc2C(=O)NC2(c3cccc4ccccc34)CC2)C1] |
| 434 | 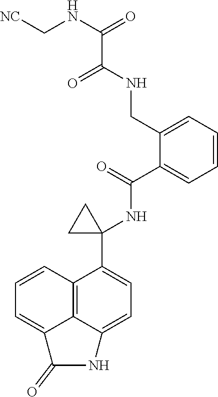 [SMILES: N#CCNC(=O)C(=O)NCc1ccccc1C(=O)NC1(c2ccc3c4c(cccc24)C(=O)N3)CC1] |
| 435 | 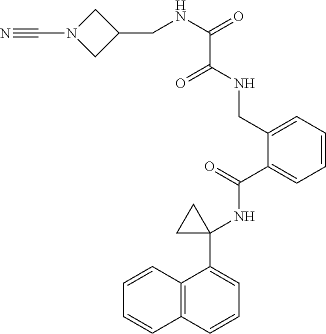 [SMILES: N#CN1CC(CNC(=O)C(=O)NCc2ccccc2C(=O)NC2(c3cccc4ccccc34)CC2)C1] |
| 436 | 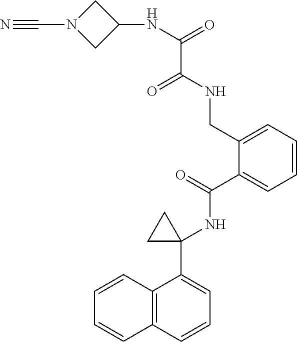 [SMILES: N#CN1CC(NC(=O)C(=O)NCc2ccccc2C(=O)NC2(c3cccc4ccccc34)CC2)C1] |
| 437 | 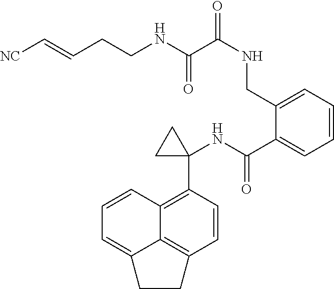 [SMILES: N#C/C=C/CCNC(=O)C(=O)NCc1ccccc1C(=O)NC1(c2ccc3c4c(cccc24)CC3)CC1] |
| 438 | 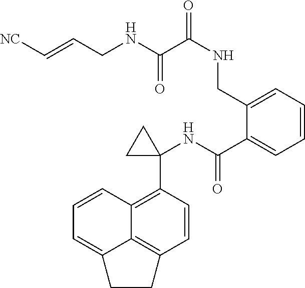 [SMILES: N#C/C=C/CNC(=O)C(=O)NCc1ccccc1C(=O)NC1(c2ccc3c4c(cccc24)CC3)CC1] |
| 439 | 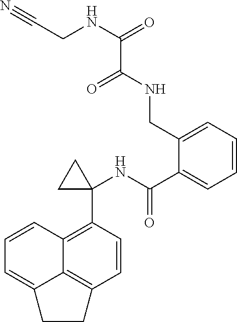 [SMILES: N#CCNC(=O)C(=O)NCc1ccccc1C(=O)NC1(c2ccc3c4c(cccc24)CC3)CC1] |
| 440 | 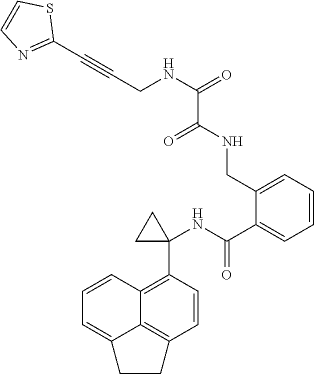 [SMILES: O=C(NCC#Cc1nccs1)C(=O)NCc1ccccc1C(=O)NC1(c2ccc3c4c(cccc24)CC3)CC1] |
| 441 | 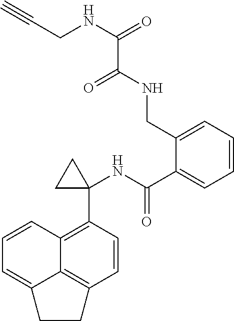 [SMILES: C#CCNC(=O)C(=O)NCc1ccccc1C(=O)NC1(c2ccc3c4c(cccc24)CC3)CC1] |
| 442 | 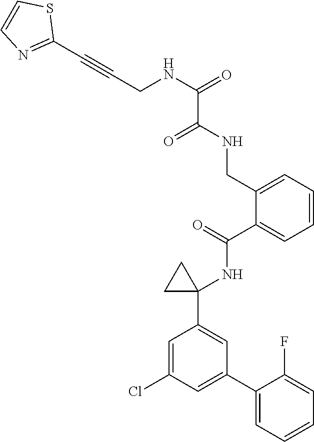 [SMILES: O=C(NCC#Cc1nccs1)C(=O)NCc1ccccc1C(=O)NC1(c2cc(Cl)cc(-c3ccccc3F)c2)CC1] |
| 443 | 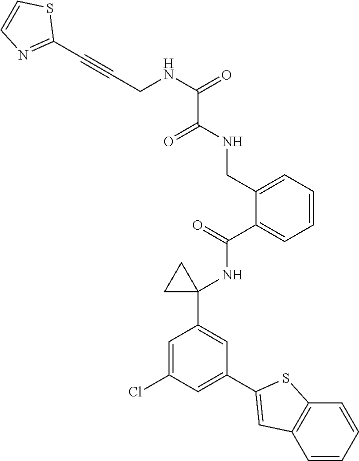 [SMILES: O=C(NCC#Cc1nccs1)C(=O)NCc1ccccc1C(=O)NC1(c2cc(Cl)cc(-c3cc4ccccc4s3)c2)CC1] |
| 444 | 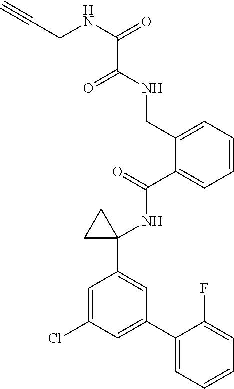 [SMILES: C#CCNC(=O)C(=O)NCc1ccccc1C(=O)NC1(c2cc(Cl)cc(-c3ccccc3F)c2)CC1] |
| 445 | 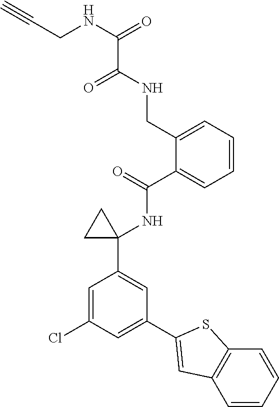 [SMILES: C#CCNC(=O)C(=O)NCc1ccccc1C(=O)NC1(c2cc(Cl)cc(-c3cc4ccccc4s3)c2)CC1] |
| 446 | 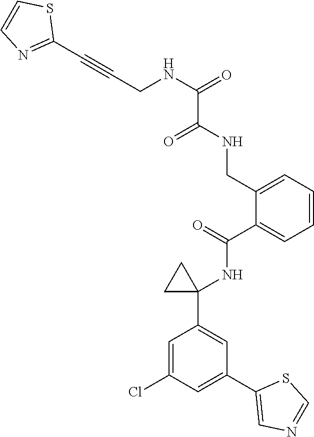 [SMILES: O=C(NCC#Cc1nccs1)C(=O)NCc1ccccc1C(=O)NC1(c2cc(Cl)cc(-c3cncs3)c2)CC1] |
| 447 | 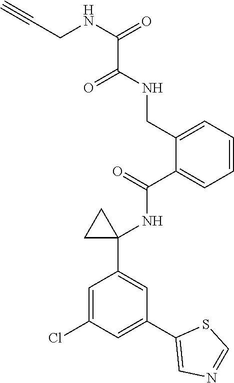 [SMILES: C#CCNC(=O)C(=O)NCc1ccccc1C(=O)NC1(c2cc(Cl)cc(-c3cncs3)c2)CC1] |
| 448 | 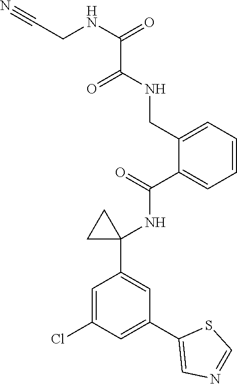 [SMILES: N#CCNC(=O)C(=O)NCc1ccccc1C(=O)NC1(c2cc(Cl)cc(-c3cncs3)c2)CC1] |
| 449 | 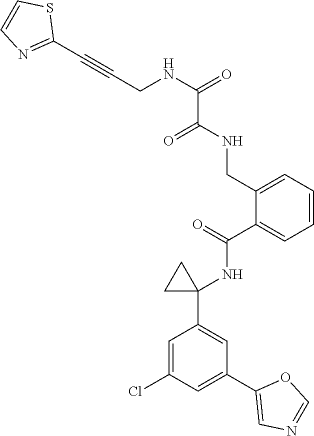 [SMILES: O=C(NCC#Cc1nccs1)C(=O)NCc1ccccc1C(=O)NC1(c2cc(Cl)cc(-c3cnco3)c2)CC1] |
| 450 | 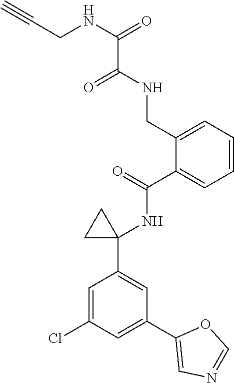 [SMILES: C#CCNC(=O)C(=O)NCc1ccccc1C(=O)NC1(c2cc(Cl)cc(-c3cnco3)c2)CC1] |
| 451 | 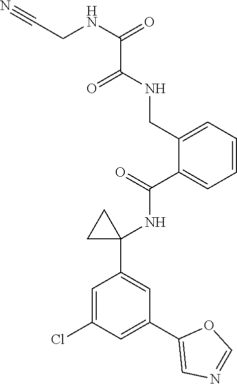 [SMILES: N#CCNC(=O)C(=O)NCc1ccccc1C(=O)NC1(c2cc(Cl)cc(-c3cnco3)c2)CC1] |
| 452 | 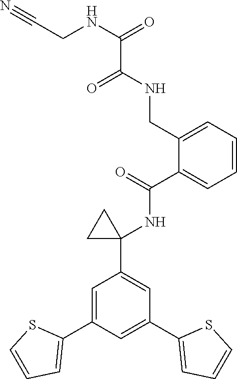 [SMILES: N#CCNC(=O)C(=O)NCc1ccccc1C(=O)NC1(c2cc(-c3cccs3)cc(-c3cccs3)c2)CC1] |
| 453 | 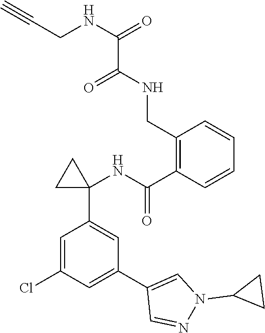 [SMILES: C#CCNC(=O)C(=O)NCc1ccccc1C(=O)NC1(c2cc(Cl)cc(-c3cnn(C4CC4)c3)c2)CC1] |
| 454 | 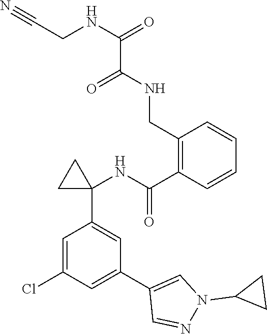 [SMILES: N#CCNC(=O)C(=O)NCc1ccccc1C(=O)NC1(c2cc(Cl)cc(-c3cnn(C4CC4)c3)c2)CC1] |
| 455 | 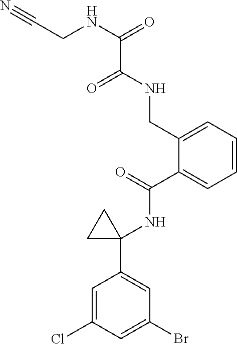 [SMILES: N#CCNC(=O)C(=O)NCc1ccccc1C(=O)NC1(c2cc(Cl)cc(Br)c2)CC1] |
| 456 | 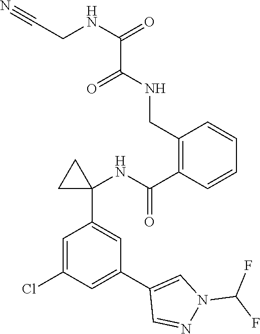 [SMILES: N#CCNC(=O)C(=O)NCc1ccccc1C(=O)NC1(c2cc(Cl)cc(-c3cnn(C(F)F)c3)c2)CC1] |
| 457 | 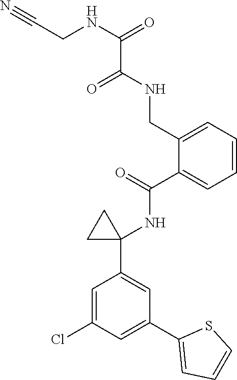 [SMILES: N#CCNC(=O)C(=O)NCc1ccccc1C(=O)NC1(c2cc(Cl)cc(-c3cccs3)c2)CC1] |
| 458 | 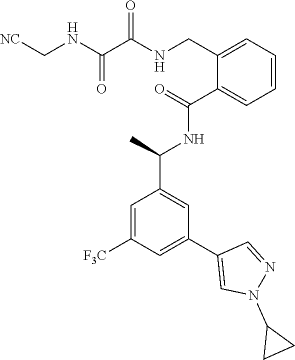 [SMILES: C[C@@H](NC(=O)c1ccccc1CNC(=O)C(=O)NCC#N)c1cc(-c2cnn(C3CC3)c2)cc(C(F)(F)F)c1] |
| 459 | 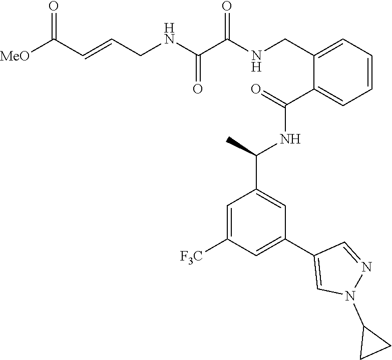 [SMILES: COC(=O)/C=C/CNC(=O)C(=O)NCc1ccccc1C(=O)N[C@H](C)c1cc(-c2cnn(C3CC3)c2)cc(C(F)(F)F)c1] |
| 460 | 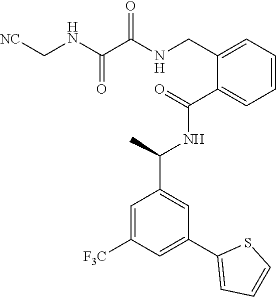 [SMILES: C[C@@H](NC(=O)c1ccccc1CNC(=O)C(=O)NCC#N)c1cc(-c2cccs2)cc(C(F)(F)F)c1] |
| 461 | 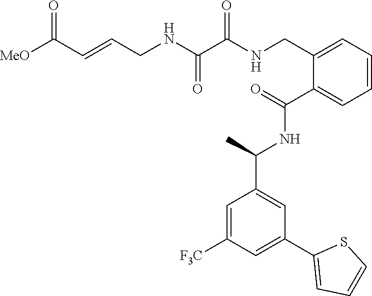 [SMILES: COC(=O)/C=C/CNC(=O)C(=O)NCc1ccccc1C(=O)N[C@H](C)c1cc(-c2cccs2)cc(C(F)(F)F)c1] |
| 462 | 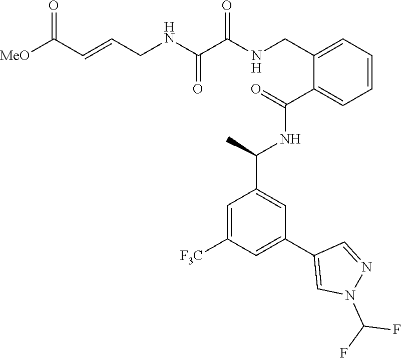 [SMILES: COC(=O)/C=C/CNC(=O)C(=O)NCc1ccccc1C(=O)N[C@H](C)c1cc(-c2cnn(C(F)F)c2)cc(C(F)(F)F)c1] |
| 463 | 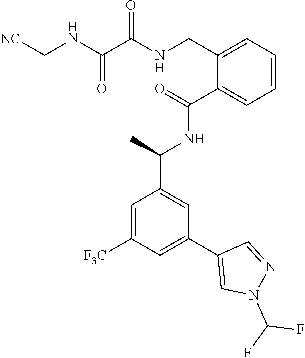 [SMILES: C[C@@H](NC(=O)c1ccccc1CNC(=O)C(=O)NCC#N)c1cc(-c2cnn(C(F)F)c2)cc(C(F)(F)F)c1] |
| 464 | 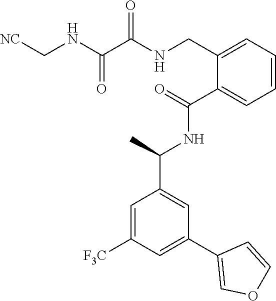 [SMILES: C[C@@H](NC(=O)c1ccccc1CNC(=O)C(=O)NCC#N)c1cc(-c2ccoc2)cc(C(F)(F)F)c1] |
| 465 | 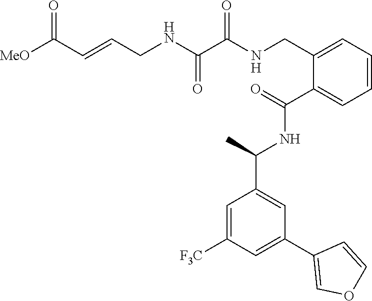 [SMILES: COC(=O)/C=C/CNC(=O)C(=O)NCc1ccccc1C(=O)N[C@H](C)c1cc(-c2ccoc2)cc(C(F)(F)F)c1] |
| 466 | 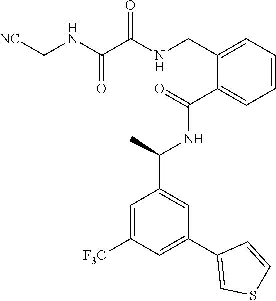 [SMILES: C[C@@H](NC(=O)c1ccccc1CNC(=O)C(=O)NCC#N)c1cc(-c2ccsc2)cc(C(F)(F)F)c1] |
| 467 | 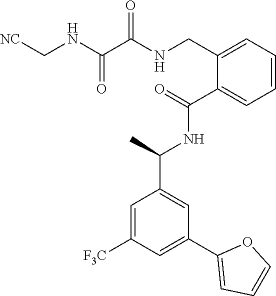 [SMILES: C[C@@H](NC(=O)c1ccccc1CNC(=O)C(=O)NCC#N)c1cc(-c2ccco2)cc(C(F)(F)F)c1] |
| 468 | 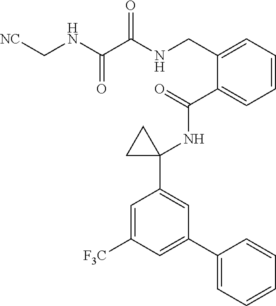 [SMILES: N#CCNC(=O)C(=O)NCc1ccccc1C(=O)NC1(c2cc(-c3ccccc3)cc(C(F)(F)F)c2)CC1] |
| 469 | 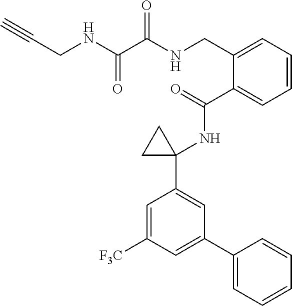 [SMILES: C#CCNC(=O)C(=O)NCc1ccccc1C(=O)NC1(c2cc(-c3ccccc3)cc(C(F)(F)F)c2)CC1] |
| 470 | 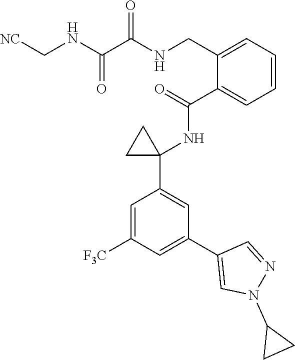 [SMILES: N#CCNC(=O)C(=O)NCc1ccccc1C(=O)NC1(c2cc(-c3cnn(C4CC4)c3)cc(C(F)(F)F)c2)CC1] |
| 471 | 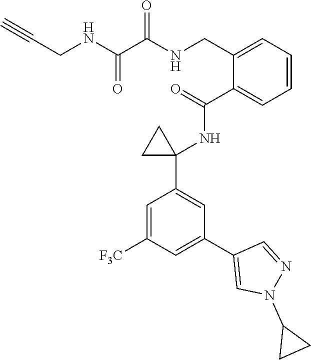 [SMILES: C#CCNC(=O)C(=O)NCc1ccccc1C(=O)NC1(c2cc(-c3cnn(C4CC4)c3)cc(C(F)(F)F)c2)CC1] |
| 472 | 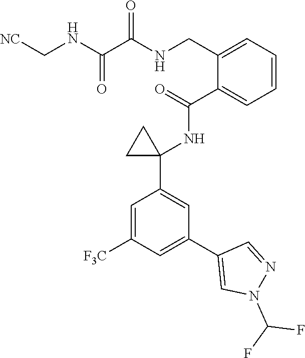 [SMILES: N#CCNC(=O)C(=O)NCc1ccccc1C(=O)NC1(c2cc(-c3cnn(C(F)F)c3)cc(C(F)(F)F)c2)CC1] |
| 473 | 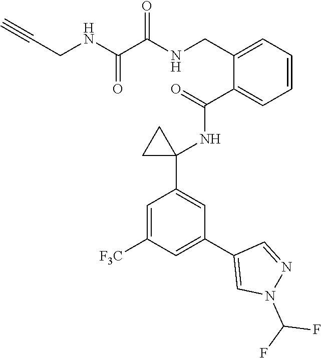 [SMILES: C#CCNC(=O)C(=O)NCc1ccccc1C(=O)NC1(c2cc(-c3cnn(C(F)F)c3)cc(C(F)(F)F)c2)CC1] |
| 474 | 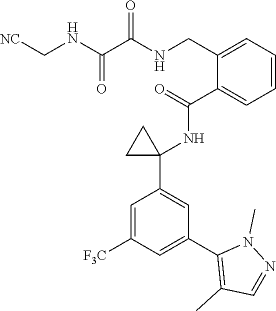 [SMILES: Cc1cnn(C)c1-c1cc(C(F)(F)F)cc(C2(NC(=O)c3ccccc3CNC(=O)C(=O)NCC#N)CC2)c1] |
| 475 | 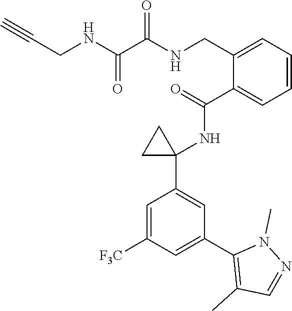 [SMILES: C#CCNC(=O)C(=O)NCc1ccccc1C(=O)NC1(c2cc(-c3c(C)cnn3C)cc(C(F)(F)F)c2)CC1] |
| 476 | 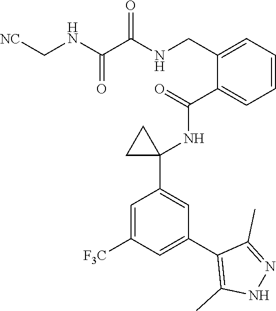 [SMILES: Cc1n[nH]c(C)c1-c1cc(C(F)(F)F)cc(C2(NC(=O)c3ccccc3CNC(=O)C(=O)NCC#N)CC2)c1] |
| 477 | 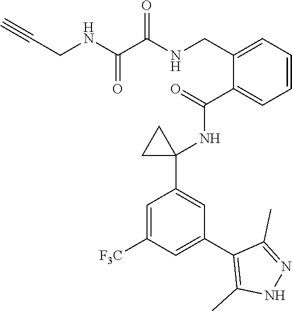 [SMILES: C#CCNC(=O)C(=O)NCc1ccccc1C(=O)NC1(c2cc(-c3c(C)n[nH]c3C)cc(C(F)(F)F)c2)CC1] |
| 478 | 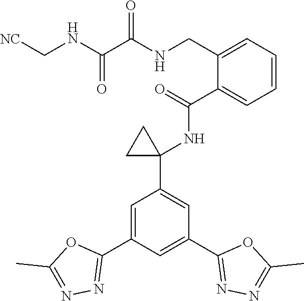 [SMILES: Cc1nnc(-c2cc(-c3nnc(C)o3)cc(C3(NC(=O)c4ccccc4CNC(=O)C(=O)NCC#N)CC3)c2)o1] |
| 479 | 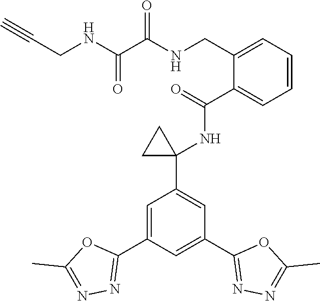 [SMILES: C#CCNC(=O)C(=O)NCc1ccccc1C(=O)NC1(c2cc(-c3nnc(C)o3)cc(-c3nnc(C)o3)c2)CC1] |
| 480 | 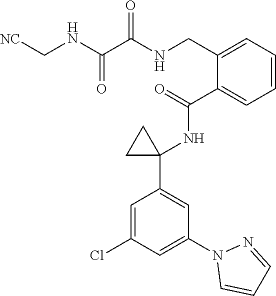 [SMILES: N#CCNC(=O)C(=O)NCc1ccccc1C(=O)NC1(c2cc(Cl)cc(-n3cccn3)c2)CC1] |
| 481 | 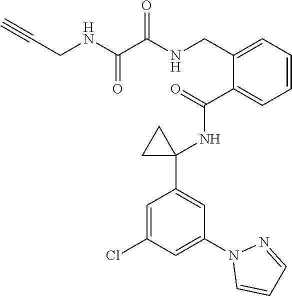 [SMILES: C#CCNC(=O)C(=O)NCc1ccccc1C(=O)NC1(c2cc(Cl)cc(-n3cccn3)c2)CC1] |
| 482 | 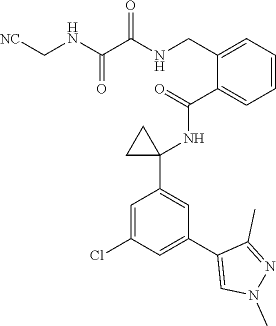 [SMILES: Cc1nn(C)cc1-c1cc(Cl)cc(C2(NC(=O)c3ccccc3CNC(=O)C(=O)NCC#N)CC2)c1] |
| 483 | 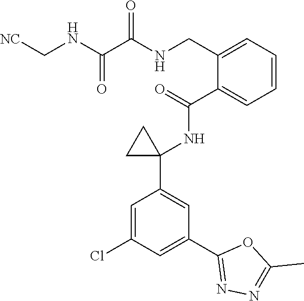 [SMILES: Cc1nnc(-c2cc(Cl)cc(C3(NC(=O)c4ccccc4CNC(=O)C(=O)NCC#N)CC3)c2)o1] |
| 484 | 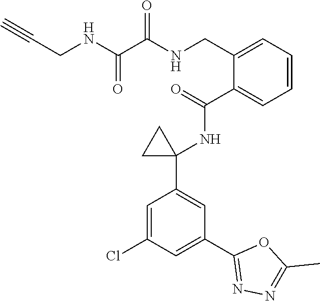 [SMILES: C#CCNC(=O)C(=O)NCc1ccccc1C(=O)NC1(c2cc(Cl)cc(-c3nnc(C)o3)c2)CC1] |
| 485 | 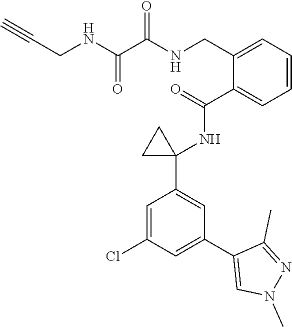 [SMILES: C#CCNC(=O)C(=O)NCc1ccccc1C(=O)NC1(c2cc(Cl)cc(-c3cn(C)nc3C)c2)CC1] |
| 486 | 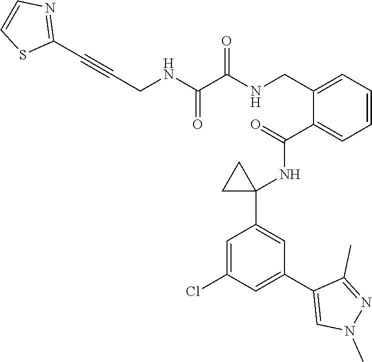 [SMILES: Cc1nn(C)cc1-c1cc(Cl)cc(C2(NC(=O)c3ccccc3CNC(=O)C(=O)NCC#Cc3nccs3)CC2)c1] |
| 487 | 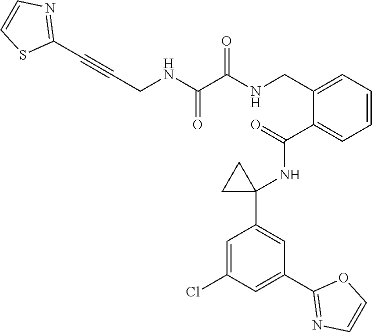 [SMILES: O=C(NCC#Cc1nccs1)C(=O)NCc1ccccc1C(=O)NC1(c2cc(Cl)cc(-c3ncco3)c2)CC1] |
| 488 | 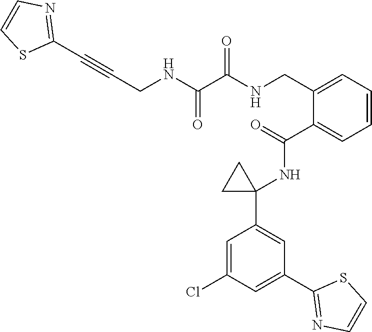 [SMILES: O=C(NCC#Cc1nccs1)C(=O)NCc1ccccc1C(=O)NC1(c2cc(Cl)cc(-c3nccs3)c2)CC1] |
| 489 | 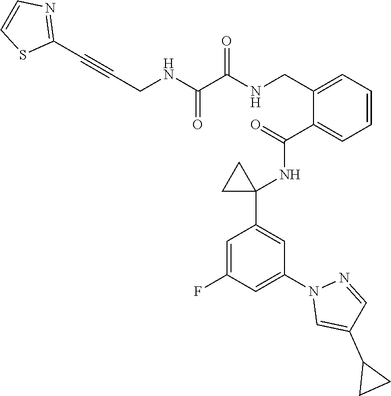 [SMILES: O=C(NCC#Cc1nccs1)C(=O)NCc1ccccc1C(=O)NC1(c2cc(F)cc(-n3cc(C4CC4)cn3)c2)CC1] |
| 490 | 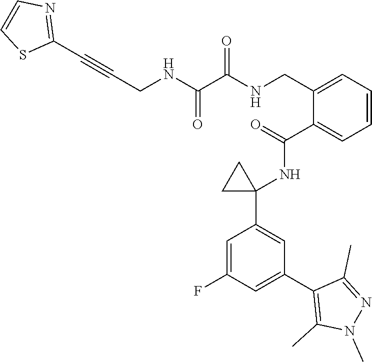 [SMILES: Cc1nn(C)c(C)c1-c1cc(F)cc(C2(NC(=O)c3ccccc3CNC(=O)C(=O)NCC#Cc3nccs3)CC2)c1] |
| 491 | 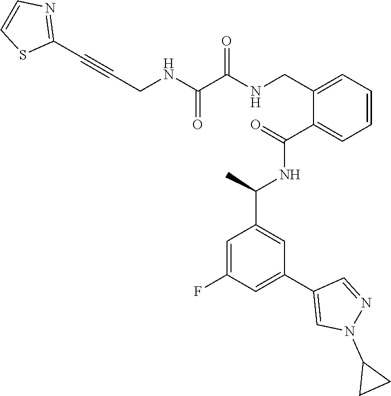 [SMILES: C[C@@H](NC(=O)c1ccccc1CNC(=O)C(=O)NCC#Cc1nccs1)c1cc(F)cc(-c2cnn(C3CC3)c2)c1] |
| 492 | 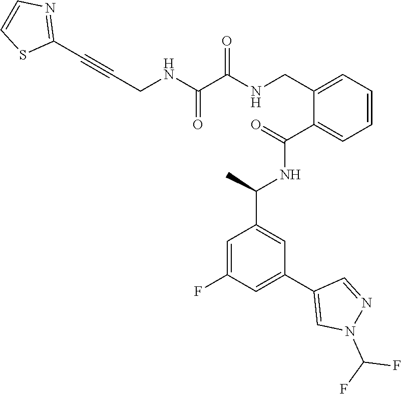 [SMILES: C[C@@H](NC(=O)c1ccccc1CNC(=O)C(=O)NCC#Cc1nccs1)c1cc(F)cc(-c2cnn(C(F)F)c2)c1] |
| 493 | 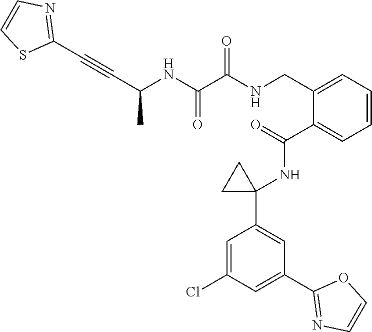 [SMILES: C[C@@H](C#Cc1nccs1)NC(=O)C(=O)NCc1ccccc1C(=O)NC1(c2cc(Cl)cc(-c3ncco3)c2)CC1] |
| 494 | 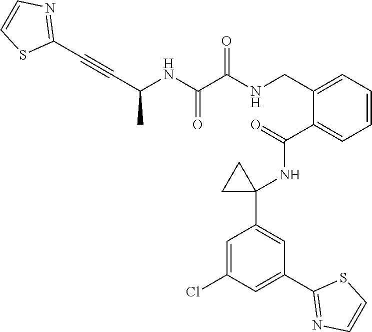 [SMILES: C[C@@H](C#Cc1nccs1)NC(=O)C(=O)NCc1ccccc1C(=O)NC1(c2cc(Cl)cc(-c3nccs3)c2)CC1] |
| 495 | 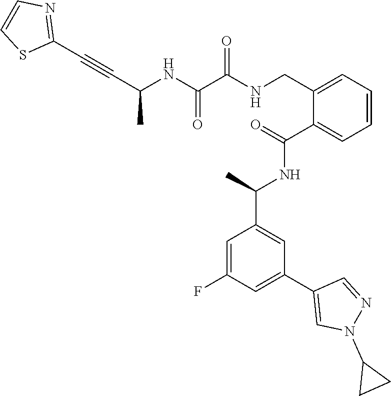 [SMILES: C[C@@H](C#Cc1nccs1)NC(=O)C(=O)NCc1ccccc1C(=O)N[C@H](C)c1cc(F)cc(-c2cnn(C3CC3)c2)c1] |
| 496 | 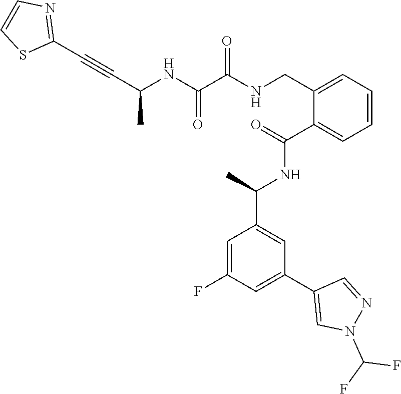 [SMILES: C[C@@H](C#Cc1nccs1)NC(=O)C(=O)NCc1ccccc1C(=O)N[C@H](C)c1cc(F)cc(-c2cnn(C(F)F)c2)c1] |
| 497 | 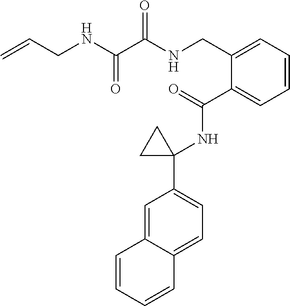 [SMILES: C=CCNC(=O)C(=O)NCc1ccccc1C(=O)NC1(c2ccc3ccccc3c2)CC1] |
| 498 | 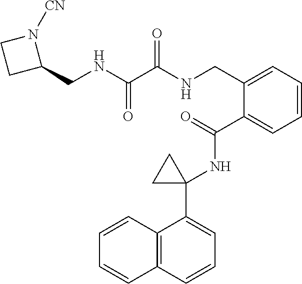 [SMILES: N#CN1CC[C@@H]1CNC(=O)C(=O)NCc1ccccc1C(=O)NC1(c2cccc3ccccc23)CC1] |
| 499 | 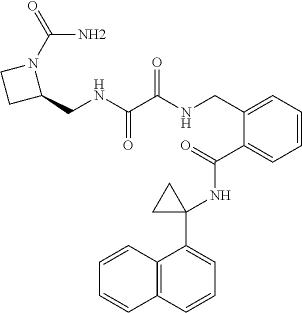 [SMILES: NC(=O)N1CC[C@@H]1CNC(=O)C(=O)NCc1ccccc1C(=O)NC1(c2cccc3ccccc23)CC1] |
| 500 | 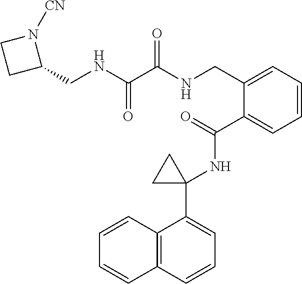 [SMILES: N#CN1CC[C@H]1CNC(=O)C(=O)NCc1ccccc1C(=O)NC1(c2cccc3ccccc23)CC1] |
| 501 | 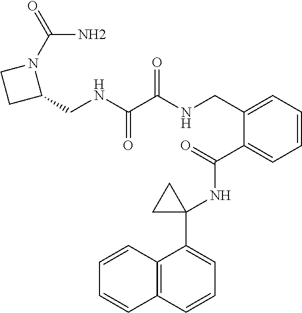 [SMILES: NC(=O)N1CC[C@H]1CNC(=O)C(=O)NCc1ccccc1C(=O)NC1(c2cccc3ccccc23)CC1] |
| 502 | 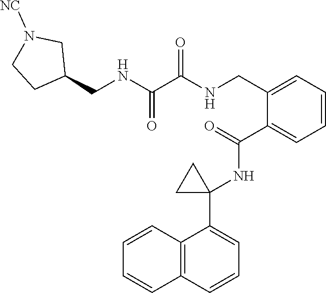 [SMILES: N#CN1CC[C@H](CNC(=O)C(=O)NCc2ccccc2C(=O)NC2(c3cccc4ccccc34)CC2)C1] |
| 503 | 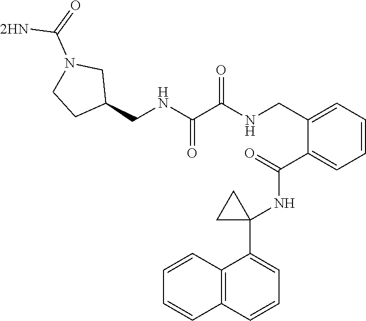 |
| 504 | 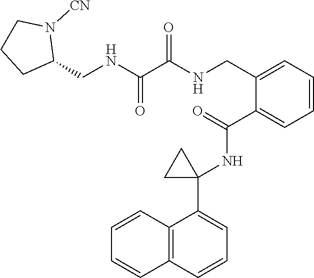 [SMILES: N#CN1CCC[C@H]1CNC(=O)C(=O)NCc1ccccc1C(=O)NC1(c2cccc3ccccc23)CC1] |
| 505 | 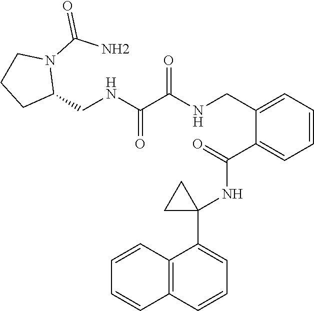 [SMILES: NC(=O)N1CCC[C@H]1CNC(=O)C(=O)NCc1ccccc1C(=O)NC1(c2cccc3ccccc23)CC1] |
| 506 | 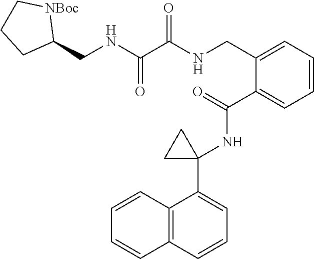 [SMILES: CC(C)(C)OC(=O)N1CCC[C@@H]1CNC(=O)C(=O)NCc1ccccc1C(=O)NC1(c2cccc3ccccc23)CC1] |
| 507 | 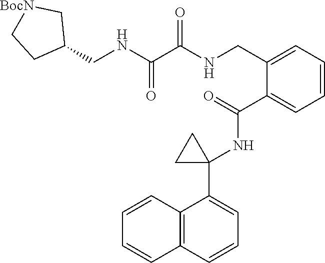 [SMILES: CC(C)(C)OC(=O)N1CC[C@@H](CNC(=O)C(=O)NCc2ccccc2C(=O)NC2(c3cccc4ccccc34)CC2)C1] |
| 508 | 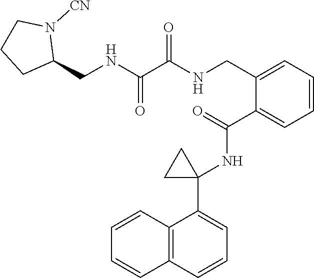 [SMILES: N#CN1CCC[C@@H]1CNC(=O)C(=O)NCc1ccccc1C(=O)NC1(c2cccc3ccccc23)CC1] |
| 509 | 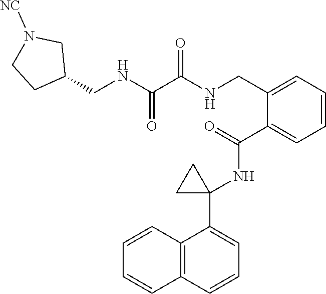 [SMILES: N#CN1CC[C@@H](CNC(=O)C(=O)NCc2ccccc2C(=O)NC2(c3cccc4ccccc34)CC2)C1] |
| 510 | 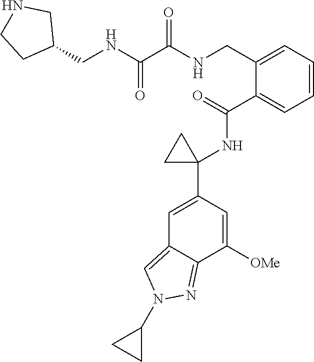 [SMILES: COc1cc(C2(NC(=O)c3ccccc3CNC(=O)C(=O)NC[C@@H]3CCNC3)CC2)cc2cn(C3CC3)nc12] |
| 511 | 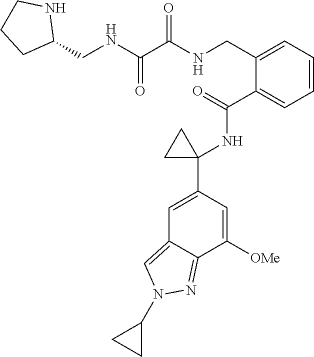 [SMILES: COc1cc(C2(NC(=O)c3ccccc3CNC(=O)C(=O)NC[C@@H]3CCCN3)CC2)cc2cn(C3CC3)nc12] |
| 512 | 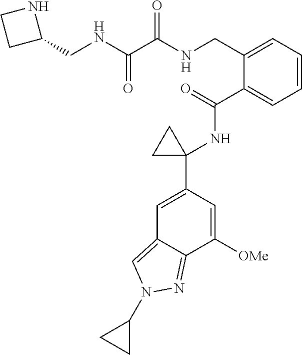 [SMILES: COc1cc(C2(NC(=O)c3ccccc3CNC(=O)C(=O)NC[C@@H]3CCN3)CC2)cc2cn(C3CC3)nc12] |
| 513 | 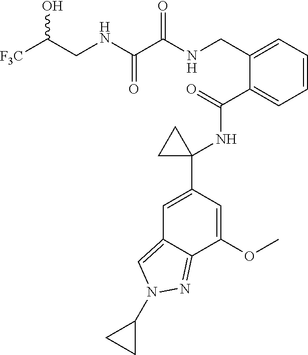 [SMILES: COc1cc(C2(NC(=O)c3ccccc3CNC(=O)C(=O)NCC(O)C(F)(F)F)CC2)cc2cn(C3CC3)nc12] |
| 514 | 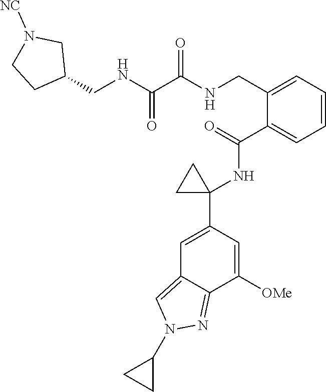 [SMILES: COc1cc(C2(NC(=O)c3ccccc3CNC(=O)C(=O)NC[C@@H]3CCN(C#N)C3)CC2)cc2cn(C3CC3)nc12] |
| 515 | 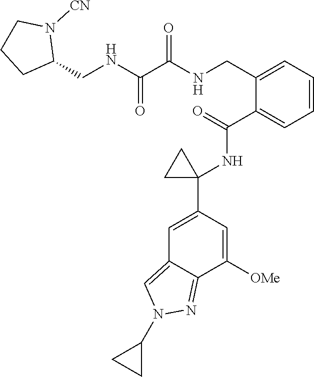 [SMILES: COc1cc(C2(NC(=O)c3ccccc3CNC(=O)C(=O)NC[C@@H]3CCCN3C#N)CC2)cc2cn(C3CC3)nc12] |
| 516 | 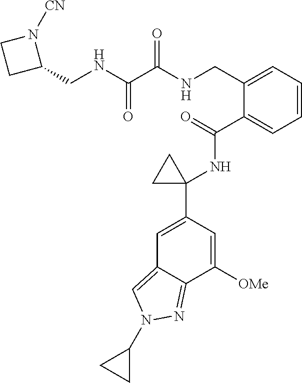 [SMILES: COc1cc(C2(NC(=O)c3ccccc3CNC(=O)C(=O)NC[C@@H]3CCN3C#N)CC2)cc2cn(C3CC3)nc12] |
| 517 | 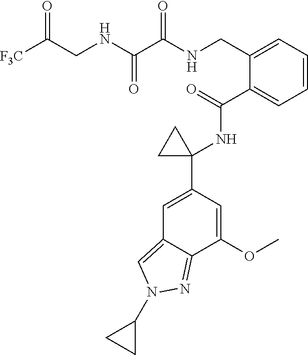 [SMILES: COc1cc(C2(NC(=O)c3ccccc3CNC(=O)C(=O)NCC(=O)C(F)(F)F)CC2)cc2cn(C3CC3)nc12] |
| 518 | 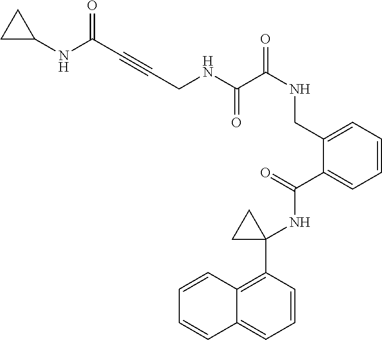 [SMILES: O=C(C#CCNC(=O)C(=O)NCc1ccccc1C(=O)NC1(c2cccc3ccccc23)CC1)NC1CC1] |
| 519 | 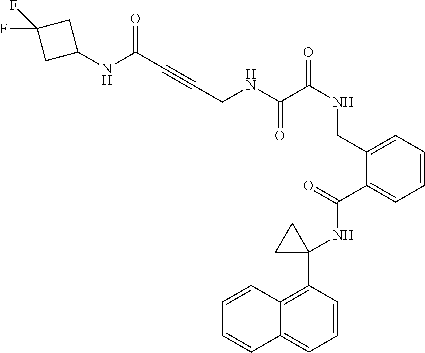 [SMILES: O=C(C#CCNC(=O)C(=O)NCc1ccccc1C(=O)NC1(c2cccc3ccccc23)CC1)NC1CC(F)(F)C1] |
| 520 | 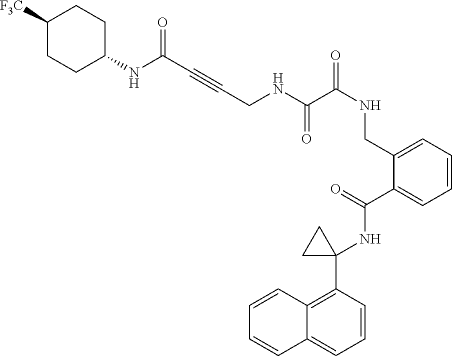 [SMILES: O=C(C#CCNC(=O)C(=O)NCc1ccccc1C(=O)NC1(c2cccc3ccccc23)CC1)N[C@H]1CC[C@H](C(F)(F)F)CC1] |
| 521 | 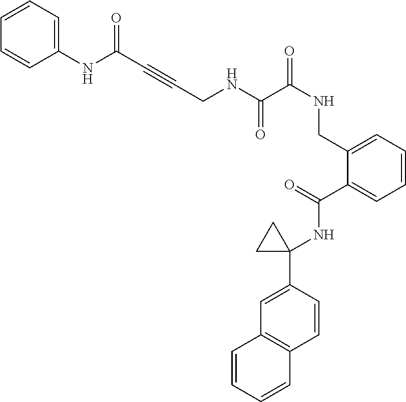 [SMILES: O=C(C#CCNC(=O)C(=O)NCc1ccccc1C(=O)NC1(c2ccc3ccccc3c2)CC1)Nc1ccccc1] |
| 522 | 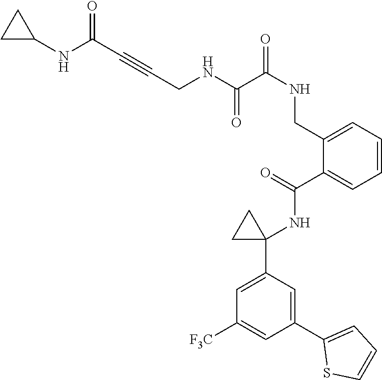 [SMILES: O=C(C#CCNC(=O)C(=O)NCc1ccccc1C(=O)NC1(c2cc(-c3cccs3)cc(C(F)(F)F)c2)CC1)NC1CC1] |
| 523 | 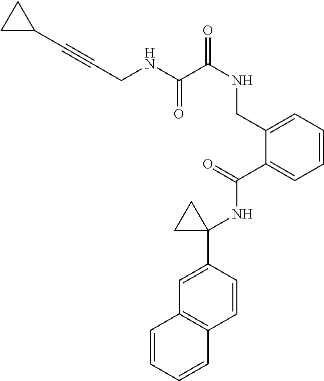 [SMILES: O=C(NCC#CC1CC1)C(=O)NCc1ccccc1C(=O)NC1(c2ccc3ccccc3c2)CC1] |
| 524 | 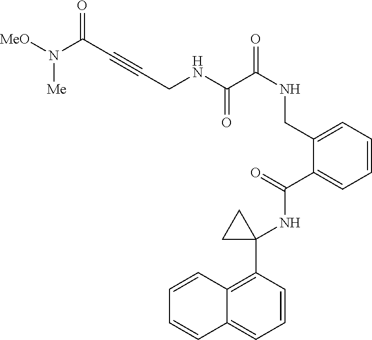 [SMILES: CON(C)C(=O)C#CCNC(=O)C(=O)NCc1ccccc1C(=O)NC1(c2cccc3ccccc23)CC1] |
| 525 | 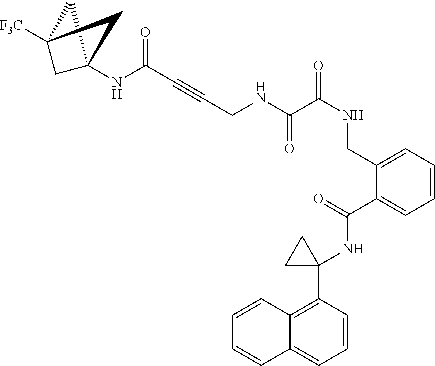 [SMILES: O=C(C#CCNC(=O)C(=O)NCc1ccccc1C(=O)NC1(c2cccc3ccccc23)CC1)N[C@]12C[C@](C(F)(F)F)(C1)C2] |
| 526 | 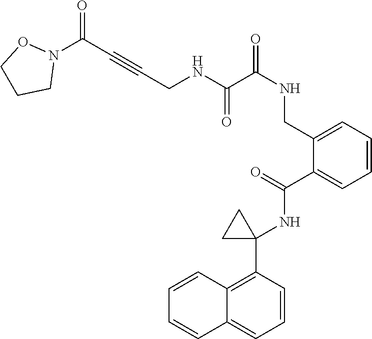 [SMILES: O=C(NCC#CC(=O)N1CCCO1)C(=O)NCc1ccccc1C(=O)NC1(c2cccc3ccccc23)CC1] |
| 527 | 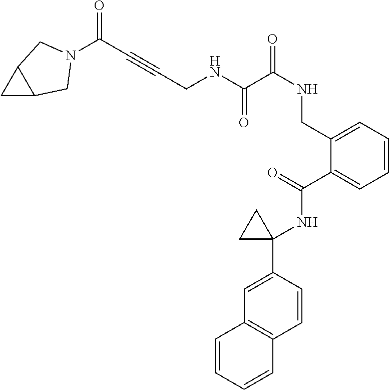 [SMILES: O=C(NCC#CC(=O)N1CC2CC2C1)C(=O)NCc1ccccc1C(=O)NC1(c2ccc3ccccc3c2)CC1] |
| 528 | 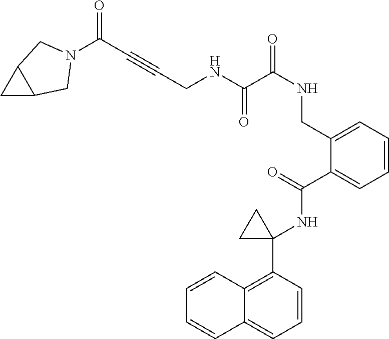 [SMILES: O=C(NCC#CC(=O)N1CC2CC2C1)C(=O)NCc1ccccc1C(=O)NC1(c2cccc3ccccc23)CC1] |
| 529 | 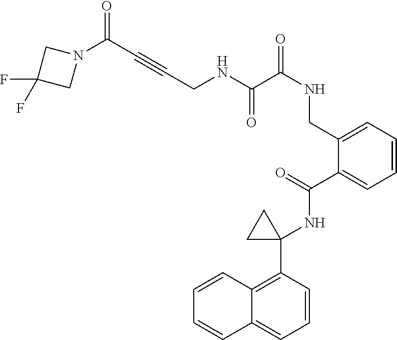 [SMILES: O=C(NCC#CC(=O)N1CC(F)(F)C1)C(=O)NCc1ccccc1C(=O)NC1(c2cccc3ccccc23)CC1] |
| 530 | 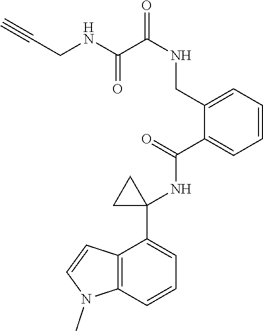 [SMILES: C#CCNC(=O)C(=O)NCc1ccccc1C(=O)NC1(c2cccc3c2ccn3C)CC1] |
| 531 | 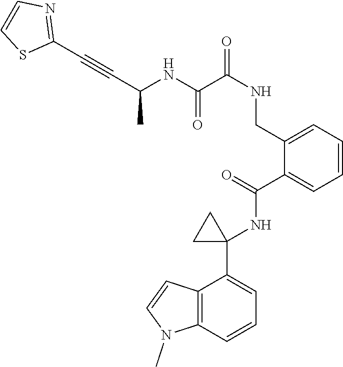 [SMILES: C[C@@H](C#Cc1nccs1)NC(=O)C(=O)NCc1ccccc1C(=O)NC1(c2cccc3c2ccn3C)CC1] |
| 532 | 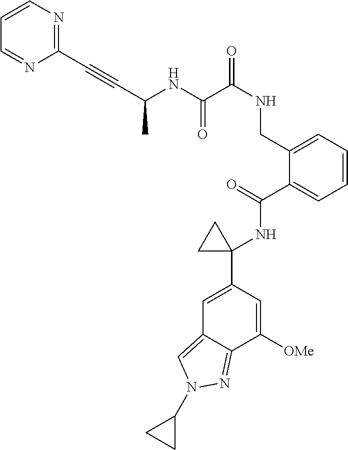 [SMILES: COc1cc(C2(NC(=O)c3ccccc3CNC(=O)C(=O)N[C@@H](C)C#Cc3ncccn3)CC2)cc2cn(C3CC3)nc12] |
| 533 | 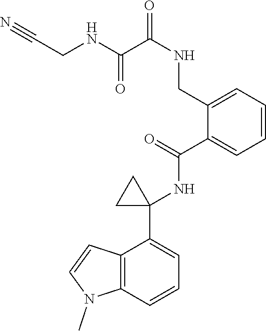 [SMILES: Cn1ccc2c(C3(NC(=O)c4ccccc4CNC(=O)C(=O)NCC#N)CC3)cccc21] |
| 534 | 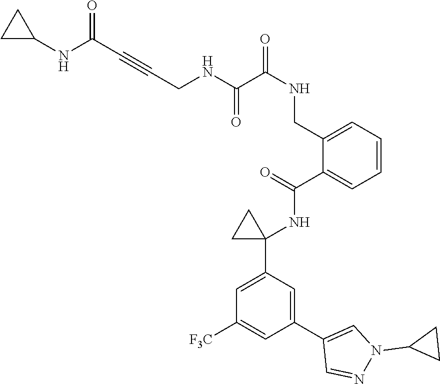 [SMILES: O=C(C#CCNC(=O)C(=O)NCc1ccccc1C(=O)NC1(c2cc(-c3cnn(C4CC4)c3)cc(C(F)(F)F)c2)CC1)NC1CC1] |
| 535 | 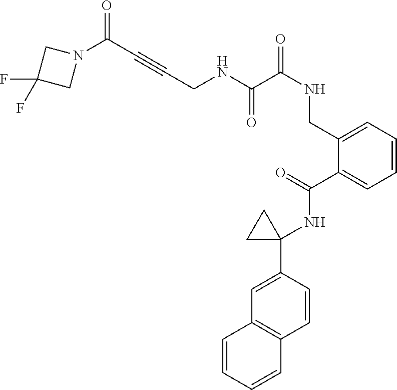 [SMILES: O=C(NCC#CC(=O)N1CC(F)(F)C1)C(=O)NCc1ccccc1C(=O)NC1(c2ccc3ccccc3c2)CC1] |
| 536 | 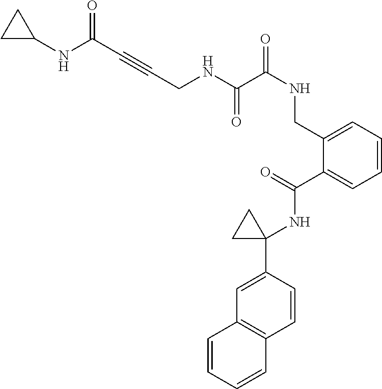 [SMILES: O=C(C#CCNC(=O)C(=O)NCc1ccccc1C(=O)NC1(c2ccc3ccccc3c2)CC1)NC1CC1] |
| 537 | 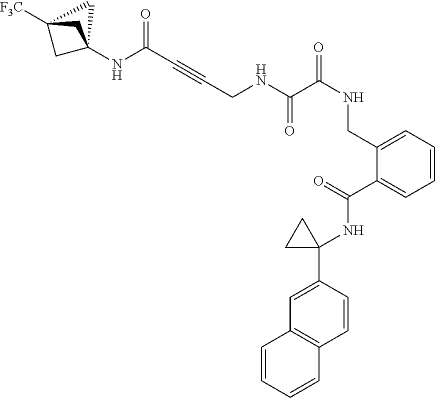 [SMILES: O=C(C#CCNC(=O)C(=O)NCc1ccccc1C(=O)NC1(c2ccc3ccccc3c2)CC1)N[C@]12C[C@](C(F)(F)F)(C1)C2] |
| 538 | 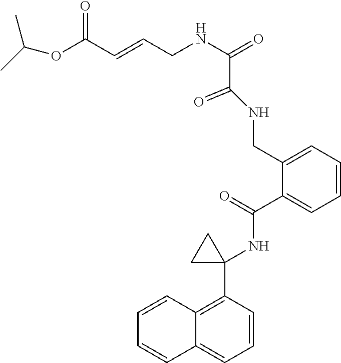 [SMILES: CC(C)OC(=O)/C=C/CNC(=O)C(=O)NCc1ccccc1C(=O)NC1(c2cccc3ccccc23)CC1] |
| 539 | 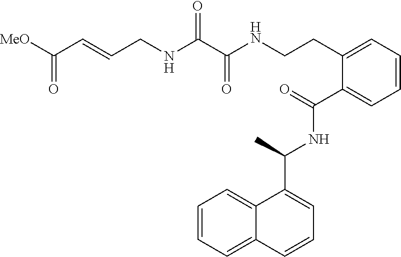 [SMILES: COC(=O)/C=C/CNC(=O)C(=O)NCCc1ccccc1C(=O)N[C@H](C)c1cccc2ccccc12] |
| 540 | 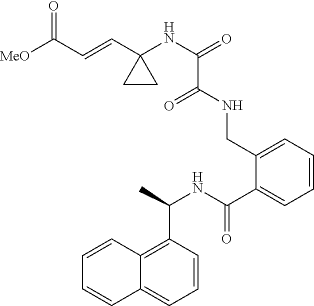 [SMILES: COC(=O)/C=C/C1(NC(=O)C(=O)NCc2ccccc2C(=O)N[C@H](C)c2cccc3ccccc23)CC1] |
| 541 | 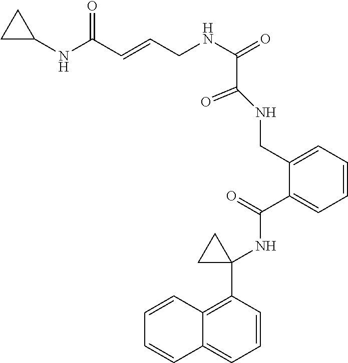 [SMILES: O=C(/C=C/CNC(=O)C(=O)NCc1ccccc1C(=O)NC1(c2cccc3ccccc23)CC1)NC1CC1] |
| 542 | 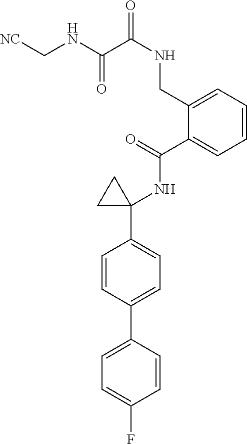 [SMILES: N#CCNC(=O)C(=O)NCc1ccccc1C(=O)NC1(c2ccc(-c3ccc(F)cc3)cc2)CC1] |
| 543 | 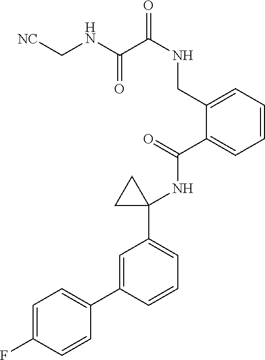 [SMILES: N#CCNC(=O)C(=O)NCc1ccccc1C(=O)NC1(c2cccc(-c3ccc(F)cc3)c2)CC1] |
| 544 | 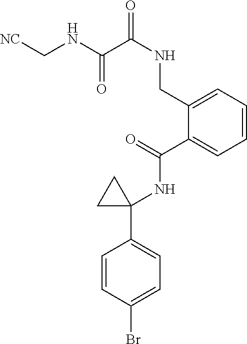 [SMILES: N#CCNC(=O)C(=O)NCc1ccccc1C(=O)NC1(c2ccc(Br)cc2)CC1] |
| 545 | 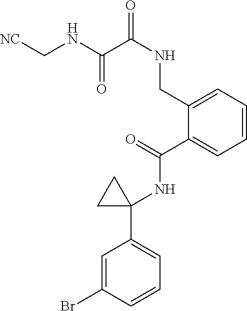 [SMILES: N#CCNC(=O)C(=O)NCc1ccccc1C(=O)NC1(c2cccc(Br)c2)CC1] |
| 546 | 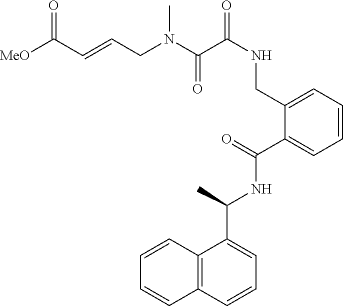 [SMILES: COC(=O)/C=C/CN(C)C(=O)C(=O)NCc1ccccc1C(=O)N[C@H](C)c1cccc2ccccc12] |
| 547 | 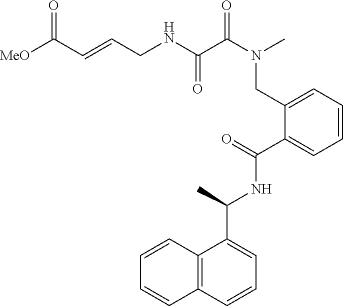 [SMILES: COC(=O)/C=C/CNC(=O)C(=O)N(C)Cc1ccccc1C(=O)N[C@H](C)c1cccc2ccccc12] |
| 548 | 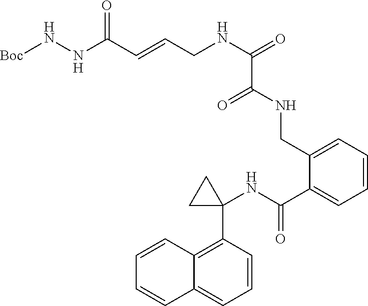 [SMILES: CC(C)(C)OC(=O)NNC(=O)/C=C/CNC(=O)C(=O)NCc1ccccc1C(=O)NC1(c2cccc3ccccc23)CC1] |
| 549 | 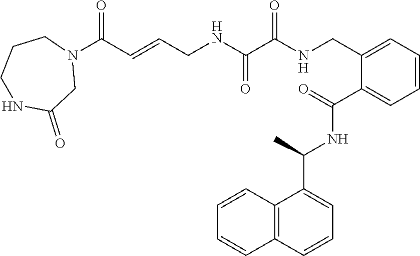 [SMILES: C[C@@H](NC(=O)c1ccccc1CNC(=O)C(=O)NC/C=C/C(=O)N1CCCNC(=O)C1)c1cccc2ccccc12] |
| 550 | 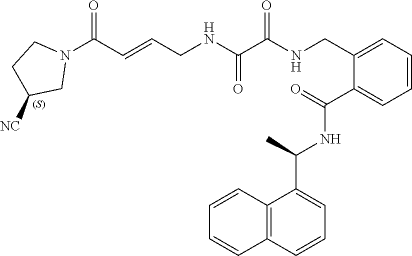 |
| 551 | 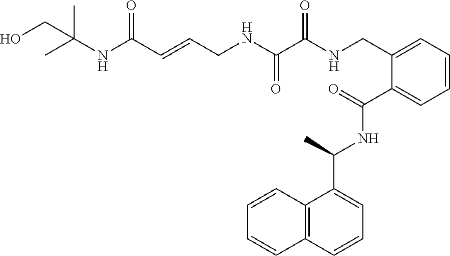 [SMILES: C[C@@H](NC(=O)c1ccccc1CNC(=O)C(=O)NC/C=C/C(=O)NC(C)(C)CO)c1cccc2ccccc12] |
| 552 | 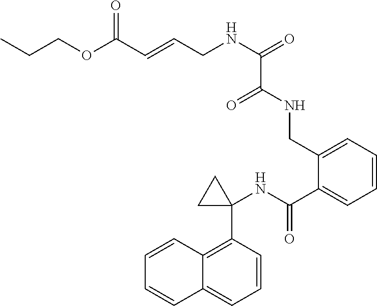 [SMILES: CCCOC(=O)/C=C/CNC(=O)C(=O)NCc1ccccc1C(=O)NC1(c2cccc3ccccc23)CC1] |
| 553 | 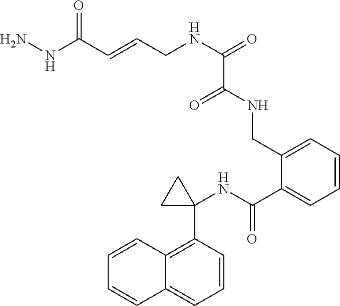 [SMILES: NNC(=O)/C=C/CNC(=O)C(=O)NCc1ccccc1C(=O)NC1(c2cccc3ccccc23)CC1] |
| 554 | 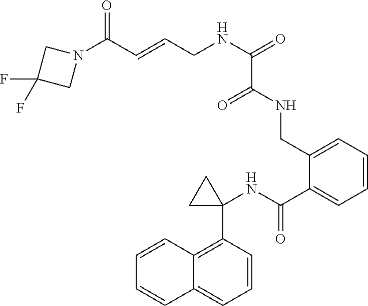 [SMILES: O=C(NC/C=C/C(=O)N1CC(F)(F)C1)C(=O)NCc1ccccc1C(=O)NC1(c2cccc3ccccc23)CC1] |
| 555 | 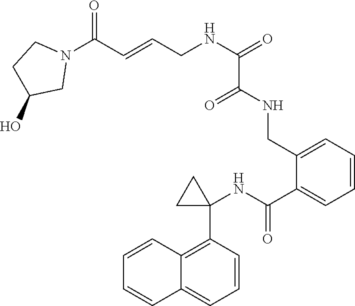 [SMILES: O=C(NC/C=C/C(=O)N1CC[C@H](O)C1)C(=O)NCc1ccccc1C(=O)NC1(c2cccc3ccccc23)CC1] |
| 556 | 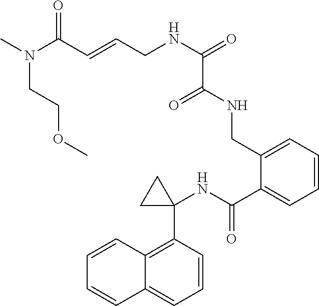 [SMILES: COCCN(C)C(=O)/C=C/CNC(=O)C(=O)NCc1ccccc1C(=O)NC1(c2cccc3ccccc23)CC1] |
| 557 | 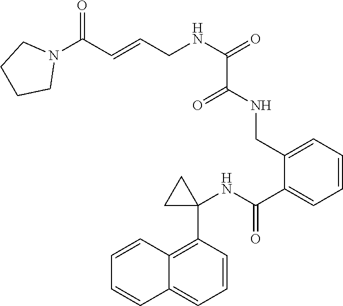 [SMILES: O=C(NC/C=C/C(=O)N1CCCC1)C(=O)NCc1ccccc1C(=O)NC1(c2cccc3ccccc23)CC1] |
| 558 | 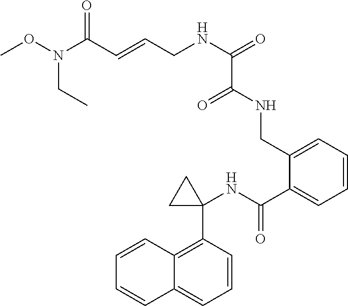 [SMILES: CCN(OC)C(=O)/C=C/CNC(=O)C(=O)NCc1ccccc1C(=O)NC1(c2cccc3ccccc23)CC1] |
| 559 | 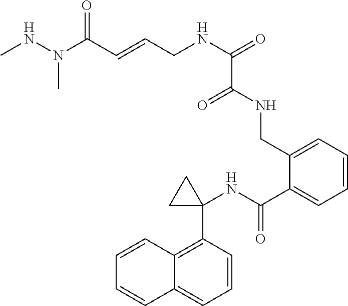 [SMILES: CNN(C)C(=O)/C=C/CNC(=O)C(=O)NCc1ccccc1C(=O)NC1(c2cccc3ccccc23)CC1] |
| 560 | 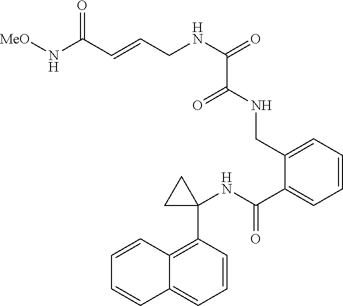 [SMILES: CONC(=O)/C=C/CNC(=O)C(=O)NCc1ccccc1C(=O)NC1(c2cccc3ccccc23)CC1] |
| 561 | 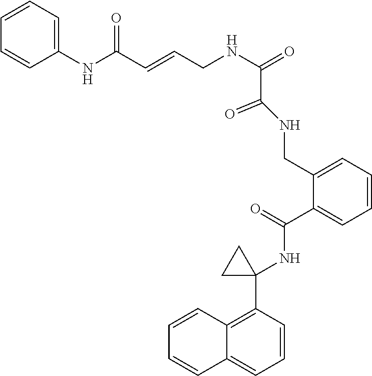 [SMILES: O=C(/C=C/CNC(=O)C(=O)NCc1ccccc1C(=O)NC1(c2cccc3ccccc23)CC1)Nc1ccccc1] |
| 562 | 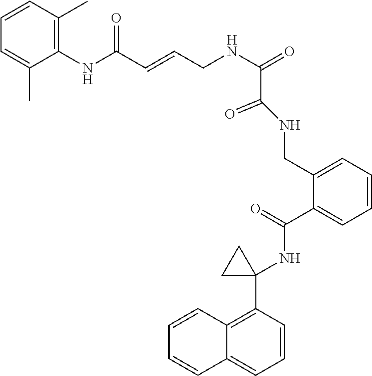 [SMILES: Cc1cccc(C)c1NC(=O)/C=C/CNC(=O)C(=O)NCc1ccccc1C(=O)NC1(c2cccc3ccccc23)CC1] |
| 563 | 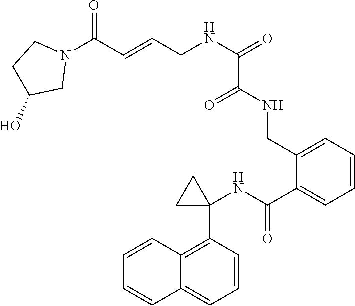 [SMILES: O=C(NC/C=C/C(=O)N1CC[C@@H](O)C1)C(=O)NCc1ccccc1C(=O)NC1(c2cccc3ccccc23)CC1] |
| 564 | 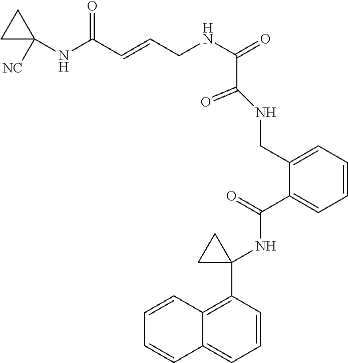 [SMILES: N#CC1(NC(=O)/C=C/CNC(=O)C(=O)NCc2ccccc2C(=O)NC2(c3cccc4ccccc34)CC2)CC1] |
| 565 | 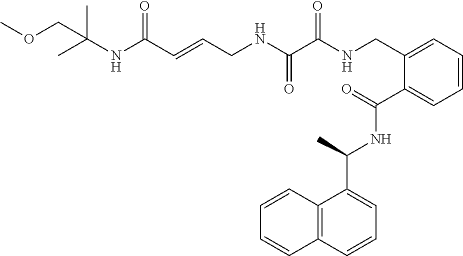 [SMILES: COCC(C)(C)NC(=O)/C=C/CNC(=O)C(=O)NCc1ccccc1C(=O)N[C@H](C)c1cccc2ccccc12] |
| 566 | 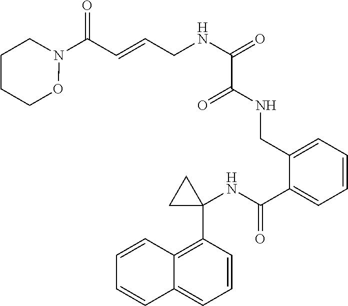 [SMILES: O=C(NC/C=C/C(=O)N1CCCCO1)C(=O)NCc1ccccc1C(=O)NC1(c2cccc3ccccc23)CC1] |
| 567 | 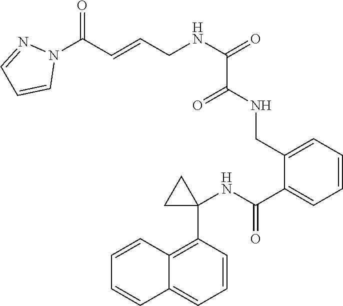 [SMILES: O=C(NC/C=C/C(=O)n1cccn1)C(=O)NCc1ccccc1C(=O)NC1(c2cccc3ccccc23)CC1] |
| 568 | 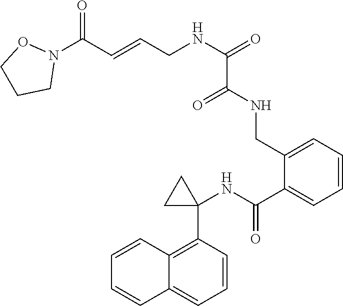 [SMILES: O=C(NC/C=C/C(=O)N1CCCO1)C(=O)NCc1ccccc1C(=O)NC1(c2cccc3ccccc23)CC1] |
| 569 | 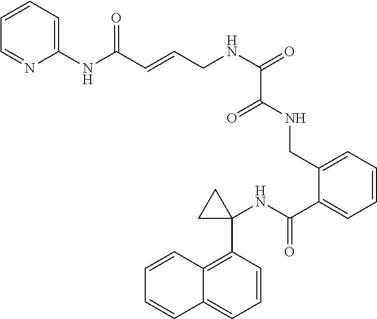 [SMILES: O=C(/C=C/CNC(=O)C(=O)NCc1ccccc1C(=O)NC1(c2cccc3ccccc23)CC1)Nc1ccccn1] |
| 571 | 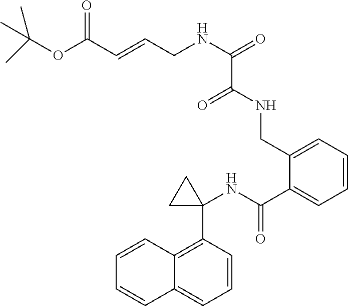 [SMILES: CC(C)(C)OC(=O)/C=C/CNC(=O)C(=O)NCc1ccccc1C(=O)NC1(c2cccc3ccccc23)CC1] |
| 572 | 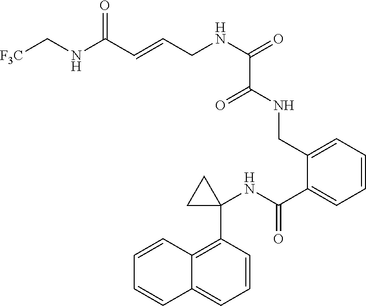 [SMILES: O=C(/C=C/CNC(=O)C(=O)NCc1ccccc1C(=O)NC1(c2cccc3ccccc23)CC1)NCC(F)(F)F] |
| 573 | 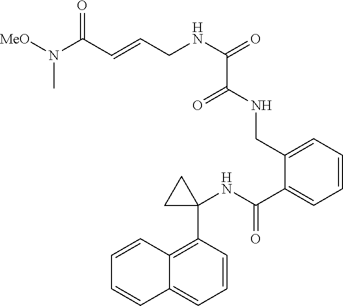 [SMILES: CON(C)C(=O)/C=C/CNC(=O)C(=O)NCc1ccccc1C(=O)NC1(c2cccc3ccccc23)CC1] |
| 574 | 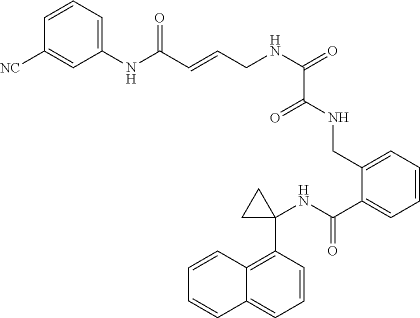 [SMILES: N#Cc1cccc(NC(=O)/C=C/CNC(=O)C(=O)NCc2ccccc2C(=O)NC2(c3cccc4ccccc34)CC2)c1] |
| 575 | 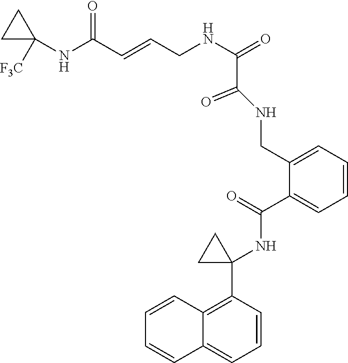 [SMILES: O=C(/C=C/CNC(=O)C(=O)NCc1ccccc1C(=O)NC1(c2cccc3ccccc23)CC1)NC1(C(F)(F)F)CC1] |
| 576 | 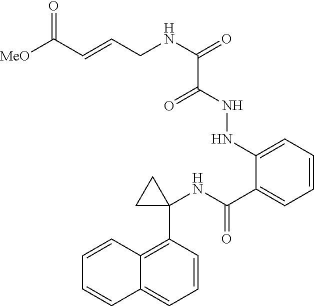 [SMILES: COC(=O)/C=C/CNC(=O)C(=O)NNc1ccccc1C(=O)NC1(c2cccc3ccccc23)CC1] |
| 577 | 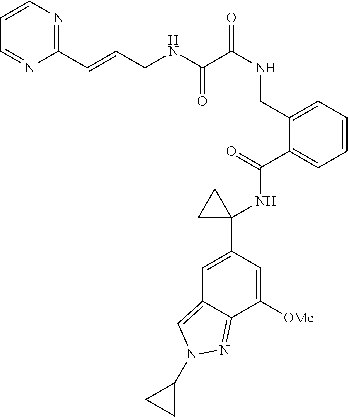 [SMILES: COc1cc(C2(NC(=O)c3ccccc3CNC(=O)C(=O)NC/C=C/c3ncccn3)CC2)cc2cn(C3CC3)nc12] |
| 578 | 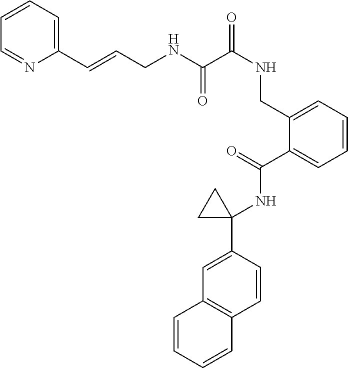 [SMILES: O=C(NC/C=C/c1ccccn1)C(=O)NCc1ccccc1C(=O)NC1(c2ccc3ccccc3c2)CC1] |
| 579 | 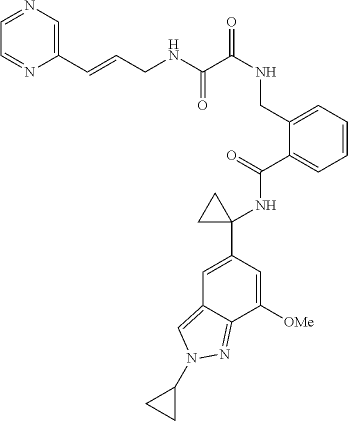 [SMILES: COc1cc(C2(NC(=O)c3ccccc3CNC(=O)C(=O)NC/C=C/c3cnccn3)CC2)cc2cn(C3CC3)nc12] |
| 580 | 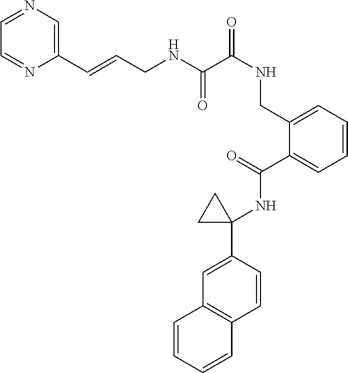 [SMILES: O=C(NC/C=C/c1cnccn1)C(=O)NCc1ccccc1C(=O)NC1(c2ccc3ccccc3c2)CC1] |
| 581 | 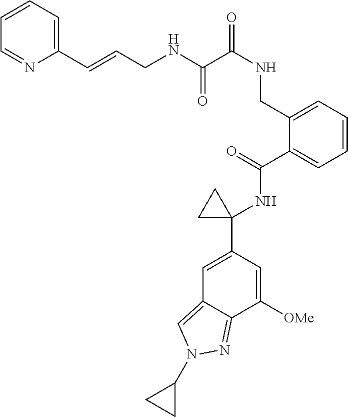 [SMILES: COc1cc(C2(NC(=O)c3ccccc3CNC(=O)C(=O)NC/C=C/c3ccccn3)CC2)cc2cn(C3CC3)nc12] |
| 582 | 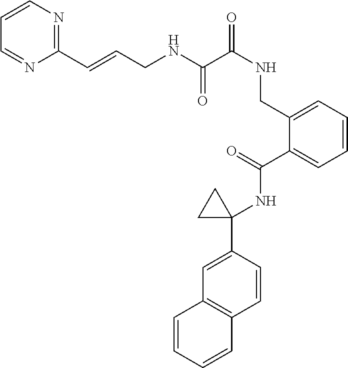 [SMILES: O=C(NC/C=C/c1ncccn1)C(=O)NCc1ccccc1C(=O)NC1(c2ccc3ccccc3c2)CC1] |
| 583 | 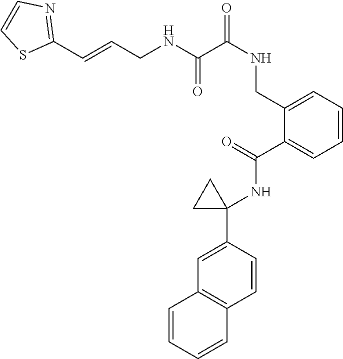 [SMILES: O=C(NC/C=C/c1nccs1)C(=O)NCc1ccccc1C(=O)NC1(c2ccc3ccccc3c2)CC1] |
| 1a | 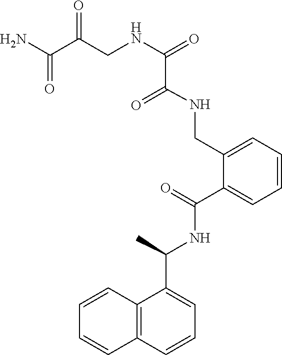 [SMILES: C[C@@H](NC(=O)c1ccccc1CNC(=O)C(=O)NCC(=O)C(N)=O)c1cccc2ccccc12] |
| 2a | 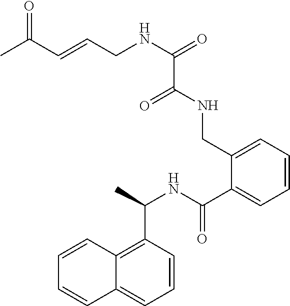 [SMILES: CC(=O)/C=C/CNC(=O)C(=O)NCc1ccccc1C(=O)N[C@H](C)c1cccc2ccccc12] |
| 3a | 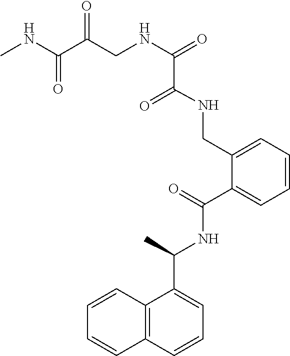 [SMILES: CNC(=O)C(=O)CNC(=O)C(=O)NCc1ccccc1C(=O)N[C@H](C)c1cccc2ccccc12] |
| 4a | 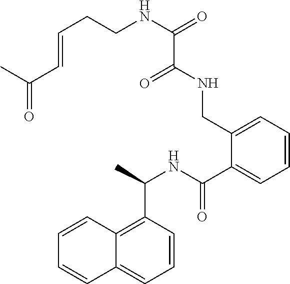 [SMILES: CC(=O)/C=C/CCNC(=O)C(=O)NCc1ccccc1C(=O)N[C@H](C)c1cccc2ccccc12] |
| 5a | 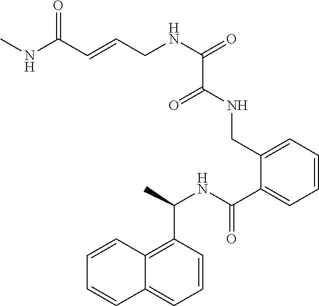 [SMILES: CNC(=O)/C=C/CNC(=O)C(=O)NCc1ccccc1C(=O)N[C@H](C)c1cccc2ccccc12] |
| 6a | 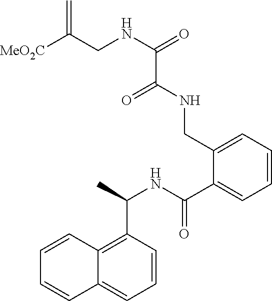 [SMILES: C=C(CNC(=O)C(=O)NCc1ccccc1C(=O)N[C@H](C)c1cccc2ccccc12)C(=O)OC] |
| 7a | 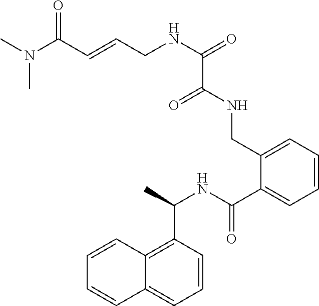 [SMILES: C[C@@H](NC(=O)c1ccccc1CNC(=O)C(=O)NC/C=C/C(=O)N(C)C)c1cccc2ccccc12] |
| 8a | 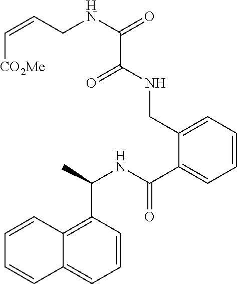 [SMILES: COC(=O)/C=C\CNC(=O)C(=O)NCc1ccccc1C(=O)N[C@H](C)c1cccc2ccccc12] |
| 9a | 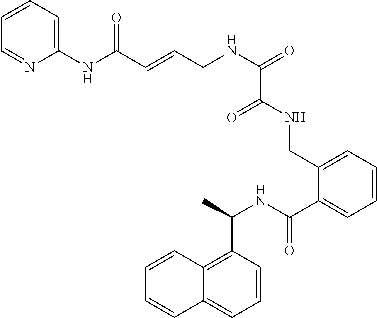 [SMILES: C[C@@H](NC(=O)c1ccccc1CNC(=O)C(=O)NC/C=C/C(=O)Nc1ccccn1)c1cccc2ccccc12] |
| 10a | 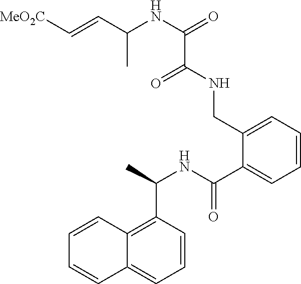 [SMILES: COC(=O)/C=C/C(C)NC(=O)C(=O)NCc1ccccc1C(=O)N[C@H](C)c1cccc2ccccc12] |
| 11a | 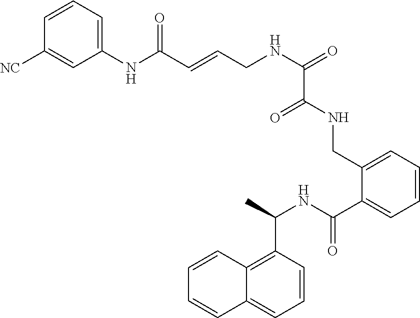 [SMILES: C[C@@H](NC(=O)c1ccccc1CNC(=O)C(=O)NC/C=C/C(=O)Nc1cccc(C#N)c1)c1cccc2ccccc12] |
| 12a | 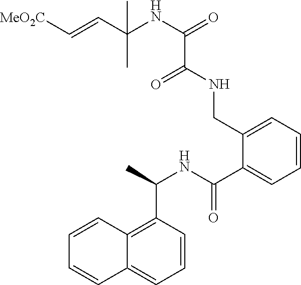 [SMILES: COC(=O)/C=C/C(C)(C)NC(=O)C(=O)NCc1ccccc1C(=O)N[C@H](C)c1cccc2ccccc12] |
| 13a | 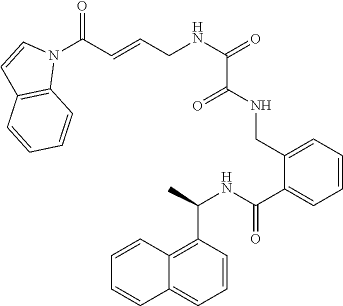 [SMILES: C[C@@H](NC(=O)c1ccccc1CNC(=O)C(=O)NC/C=C/C(=O)n1ccc2ccccc21)c1cccc2ccccc12] |
| 14a | 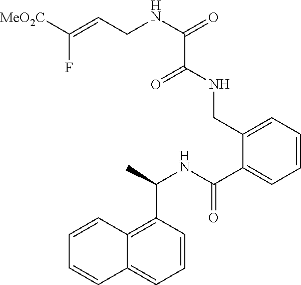 [SMILES: COC(=O)/C(F)=C/CNC(=O)C(=O)NCc1ccccc1C(=O)N[C@H](C)c1cccc2ccccc12] |
| 15a | 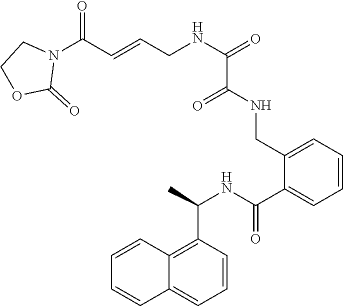 [SMILES: C[C@@H](NC(=O)c1ccccc1CNC(=O)C(=O)NC/C=C/C(=O)N1CCOC1=O)c1cccc2ccccc12] |
| 16a | 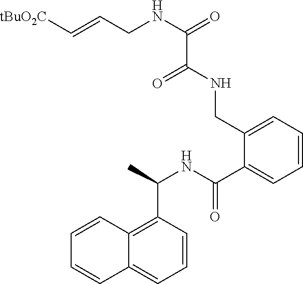 [SMILES: C[C@@H](NC(=O)c1ccccc1CNC(=O)C(=O)NC/C=C/C(=O)OC(C)(C)C)c1cccc2ccccc12] |
| 17a | 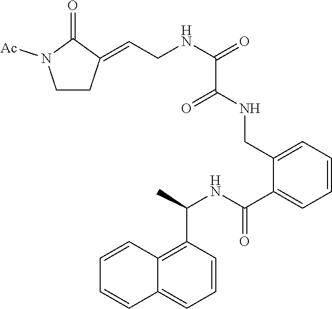 [SMILES: CC(=O)N1CC/C(=C\CNC(=O)C(=O)NCc2ccccc2C(=O)N[C@H](C)c2cccc3ccccc23)C1=O] |
| 18a | 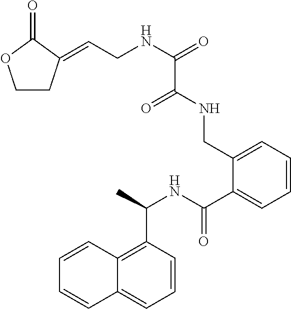 [SMILES: C[C@@H](NC(=O)c1ccccc1CNC(=O)C(=O)NC/C=C1\CCOC1=O)c1cccc2ccccc12] |
| 19a | 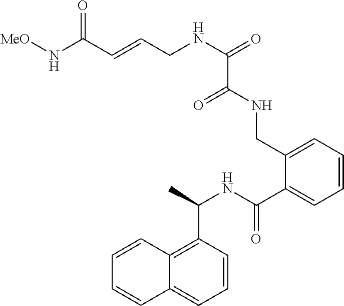 [SMILES: CONC(=O)/C=C/CNC(=O)C(=O)NCc1ccccc1C(=O)N[C@H](C)c1cccc2ccccc12] |
| 20a | 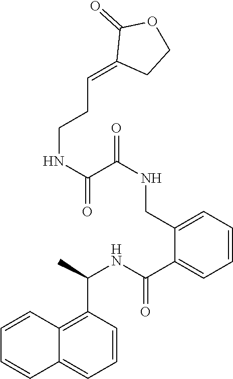 [SMILES: C[C@@H](NC(=O)c1ccccc1CNC(=O)C(=O)NCC/C=C1\CCOC1=O)c1cccc2ccccc12] |
| 21a | 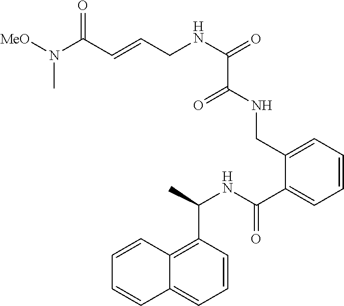 [SMILES: CON(C)C(=O)/C=C/CNC(=O)C(=O)NCc1ccccc1C(=O)N[C@H](C)c1cccc2ccccc12] |
| 22a | 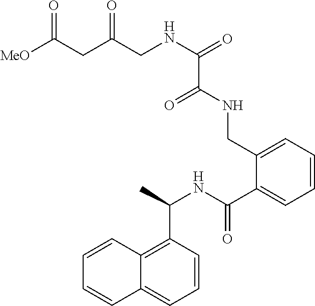 [SMILES: COC(=O)CC(=O)CNC(=O)C(=O)NCc1ccccc1C(=O)N[C@H](C)c1cccc2ccccc12] |
| 23a | 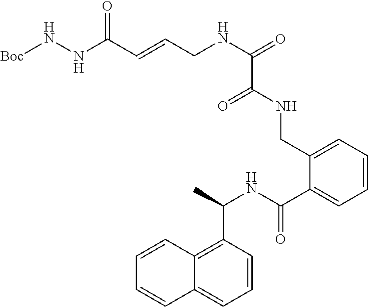 [SMILES: C[C@@H](NC(=O)c1ccccc1CNC(=O)C(=O)NC/C=C/C(=O)NNC(=O)OC(C)(C)C)c1cccc2ccccc12] |
| 24a | 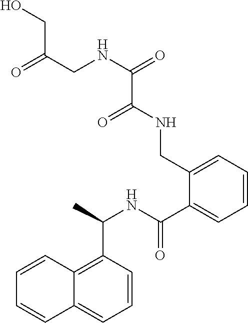 [SMILES: C[C@@H](NC(=O)c1ccccc1CNC(=O)C(=O)NCC(=O)CO)c1cccc2ccccc12] |
| 25a | 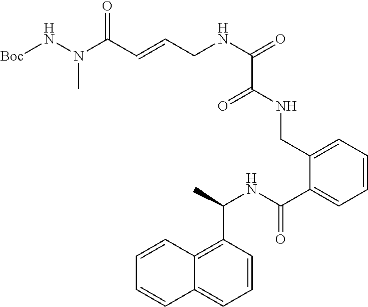 [SMILES: C[C@@H](NC(=O)c1ccccc1CNC(=O)C(=O)NC/C=C/C(=O)N(C)NC(=O)OC(C)(C)C)c1cccc2ccccc12] |
| 26a | 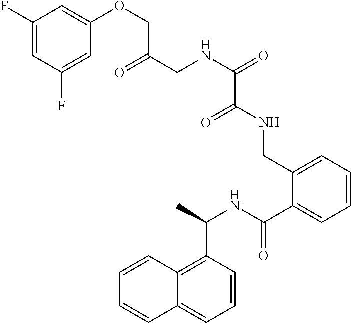 [SMILES: C[C@@H](NC(=O)c1ccccc1CNC(=O)C(=O)NCC(=O)COc1cc(F)cc(F)c1)c1cccc2ccccc12] |
| 27a | 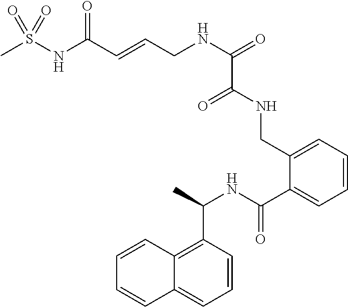 [SMILES: C[C@@H](NC(=O)c1ccccc1CNC(=O)C(=O)NC/C=C/C(=O)NS(C)(=O)=O)c1cccc2ccccc12] |
| 28a | 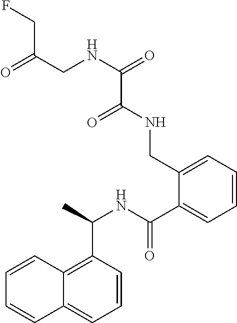 [SMILES: C[C@@H](NC(=O)c1ccccc1CNC(=O)C(=O)NCC(=O)CF)c1cccc2ccccc12] |
| 29a | 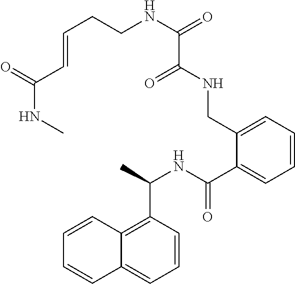 [SMILES: CNC(=O)/C=C/CCNC(=O)C(=O)NCc1ccccc1C(=O)N[C@H](C)c1cccc2ccccc12] |
| 30a | 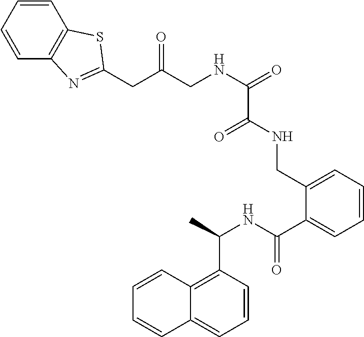 [SMILES: C[C@@H](NC(=O)c1ccccc1CNC(=O)C(=O)NCC(=O)Cc1nc2ccccc2s1)c1cccc2ccccc12] |
| 31a | 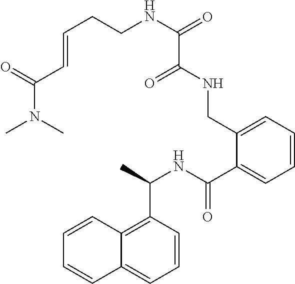 [SMILES: C[C@@H](NC(=O)c1ccccc1CNC(=O)C(=O)NCC/C=C/C(=O)N(C)C)c1cccc2ccccc12] |
| 32a | 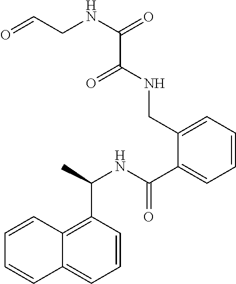 [SMILES: C[C@@H](NC(=O)c1ccccc1CNC(=O)C(=O)NCC=O)c1cccc2ccccc12] |
| 33a | 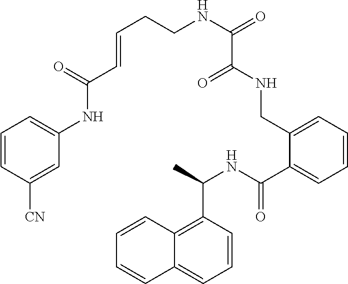 [SMILES: C[C@@H](NC(=O)c1ccccc1CNC(=O)C(=O)NCC/C=C/C(=O)Nc1cccc(C#N)c1)c1cccc2ccccc12] |
| 34a | 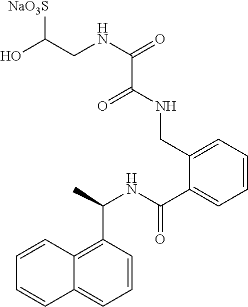 |
| 35a | 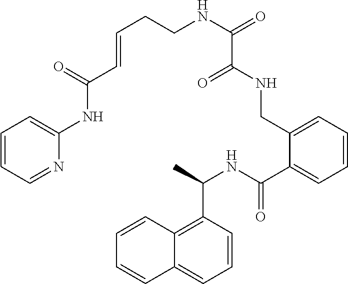 [SMILES: C[C@@H](NC(=O)c1ccccc1CNC(=O)C(=O)NCC/C=C/C(=O)Nc1ccccn1)c1cccc2ccccc12] |
| 36a | 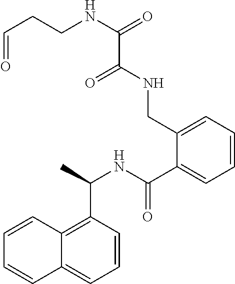 [SMILES: C[C@@H](NC(=O)c1ccccc1CNC(=O)C(=O)NCCC=O)c1cccc2ccccc12] |
| 37a | 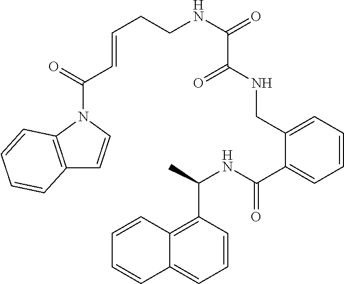 [SMILES: C[C@@H](NC(=O)c1ccccc1CNC(=O)C(=O)NCC/C=C/C(=O)n1ccc2ccccc21)c1cccc2ccccc12] |
| 38a | 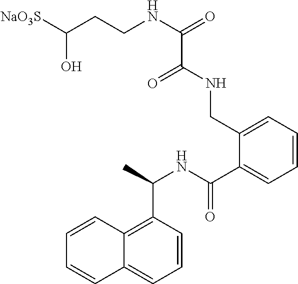 |
| 39a | 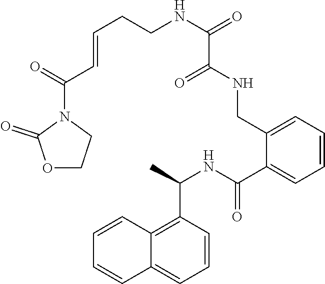 [SMILES: C[C@@H](NC(=O)c1ccccc1CNC(=O)C(=O)NCC/C=C/C(=O)N1CCOC1=O)c1cccc2ccccc12] |
| 40a | 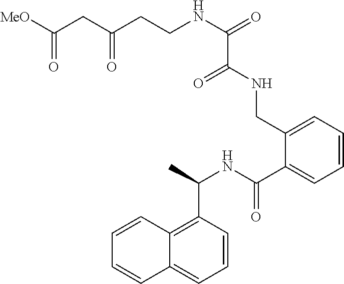 [SMILES: COC(=O)CC(=O)CCNC(=O)C(=O)NCc1ccccc1C(=O)N[C@H](C)c1cccc2ccccc12] |
| 41a | 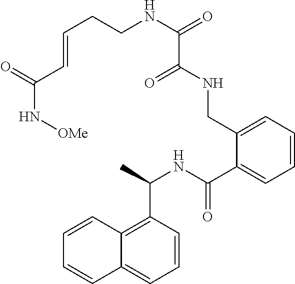 [SMILES: CONC(=O)/C=C/CCNC(=O)C(=O)NCc1ccccc1C(=O)N[C@H](C)c1cccc2ccccc12] |
| 42a | 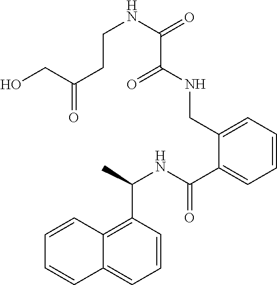 [SMILES: C[C@@H](NC(=O)c1ccccc1CNC(=O)C(=O)NCCC(=O)CO)c1cccc2ccccc12] |
| 43a | 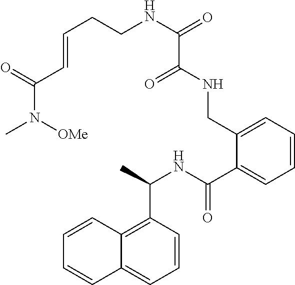 [SMILES: CON(C)C(=O)/C=C/CCNC(=O)C(=O)NCc1ccccc1C(=O)N[C@H](C)c1cccc2ccccc12] |
| 44a | 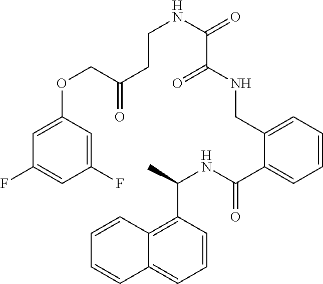 [SMILES: C[C@@H](NC(=O)c1ccccc1CNC(=O)C(=O)NCCC(=O)COc1cc(F)cc(F)c1)c1cccc2ccccc12] |
| 45a | 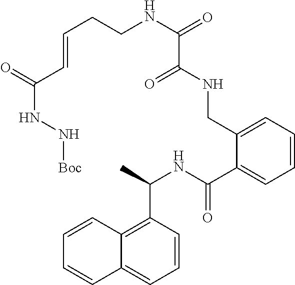 [SMILES: C[C@@H](NC(=O)c1ccccc1CNC(=O)C(=O)NCC/C=C/C(=O)NNC(=O)OC(C)(C)C)c1cccc2ccccc12] |
| 46a | 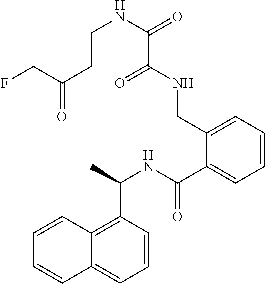 [SMILES: C[C@@H](NC(=O)c1ccccc1CNC(=O)C(=O)NCCC(=O)CF)c1cccc2ccccc12] |
| 47a | 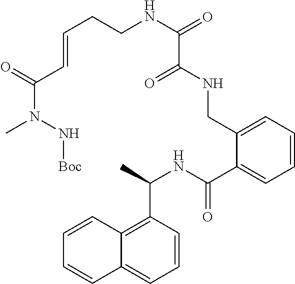 [SMILES: C[C@@H](NC(=O)c1ccccc1CNC(=O)C(=O)NCC/C=C/C(=O)N(C)NC(=O)OC(C)(C)C)c1cccc2ccccc12] |
| 48a | 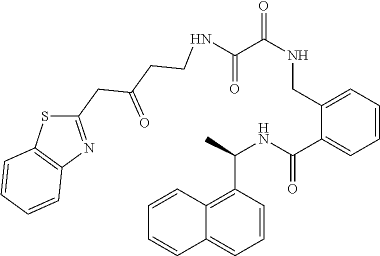 [SMILES: C[C@@H](NC(=O)c1ccccc1CNC(=O)C(=O)NCCC(=O)Cc1nc2ccccc2s1)c1cccc2ccccc12] |
| 49a | 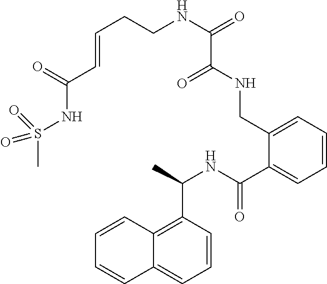 [SMILES: C[C@@H](NC(=O)c1ccccc1CNC(=O)C(=O)NCC/C=C/C(=O)NS(C)(=O)=O)c1cccc2ccccc12] |
| 50a | 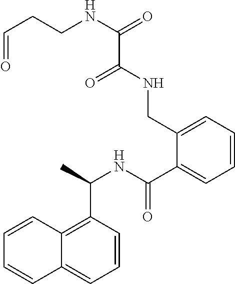 [SMILES: C[C@@H](NC(=O)c1ccccc1CNC(=O)C(=O)NCCC=O)c1cccc2ccccc12] |
| 51a | 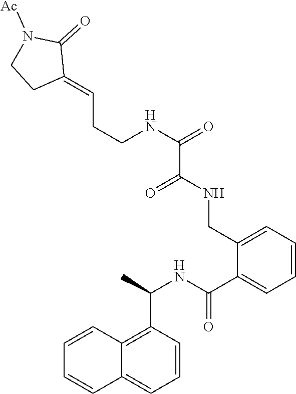 [SMILES: CC(=O)N1CC/C(=C\CCNC(=O)C(=O)NCc2ccccc2C(=O)N[C@H](C)c2cccc3ccccc23)C1=O] |
| 52a | 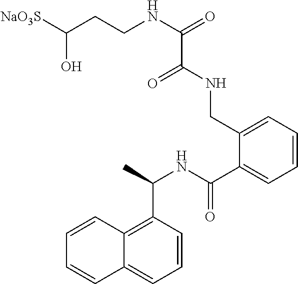 |
| 53a | 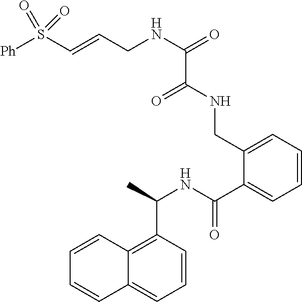 [SMILES: C[C@@H](NC(=O)c1ccccc1CNC(=O)C(=O)NC/C=C/S(=O)(=O)c1ccccc1)c1cccc2ccccc12] |
| 54a | 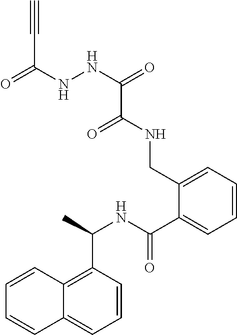 [SMILES: C#CC(=O)NNC(=O)C(=O)NCc1ccccc1C(=O)N[C@H](C)c1cccc2ccccc12] |
| 55a | 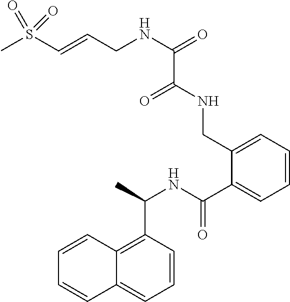 [SMILES: C[C@@H](NC(=O)c1ccccc1CNC(=O)C(=O)NC/C=C/S(C)(=O)=O)c1cccc2ccccc12] |
| 56a | 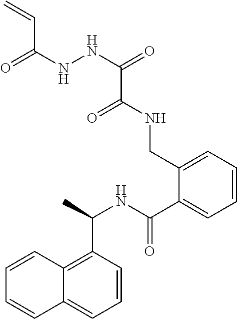 [SMILES: C=CC(=O)NNC(=O)C(=O)NCc1ccccc1C(=O)N[C@H](C)c1cccc2ccccc12] |
| 57a | 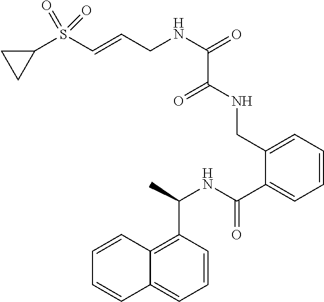 [SMILES: C[C@@H](NC(=O)c1ccccc1CNC(=O)C(=O)NC/C=C/S(=O)(=O)C1CC1)c1cccc2ccccc12] |
| 58a | 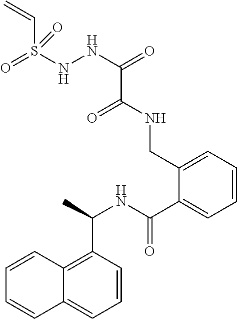 [SMILES: C=CS(=O)(=O)NNC(=O)C(=O)NCc1ccccc1C(=O)N[C@H](C)c1cccc2ccccc12] |
| 59a | 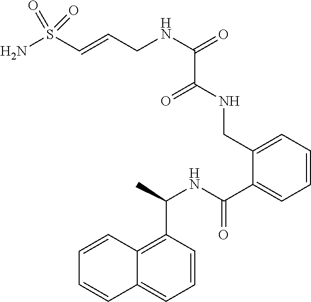 [SMILES: C[C@@H](NC(=O)c1ccccc1CNC(=O)C(=O)NC/C=C/S(N)(=O)=O)c1cccc2ccccc12] |
| 60a | 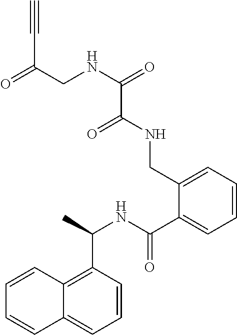 [SMILES: C#CC(=O)CNC(=O)C(=O)NCc1ccccc1C(=O)N[C@H](C)c1cccc2ccccc12] |
| 61a | 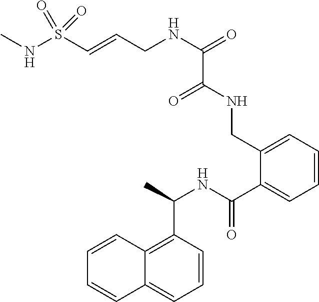 [SMILES: CNS(=O)(=O)/C=C/CNC(=O)C(=O)NCc1ccccc1C(=O)N[C@H](C)c1cccc2ccccc12] |
| 62a | 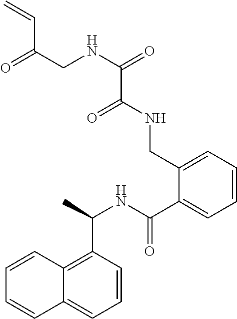 [SMILES: C=CC(=O)CNC(=O)C(=O)NCc1ccccc1C(=O)N[C@H](C)c1cccc2ccccc12] |
| 63a | 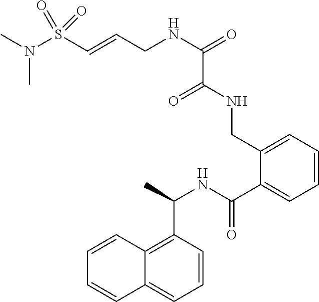 [SMILES: C[C@@H](NC(=O)c1ccccc1CNC(=O)C(=O)NC/C=C/S(=O)(=O)N(C)C)c1cccc2ccccc12] |
| 64a | 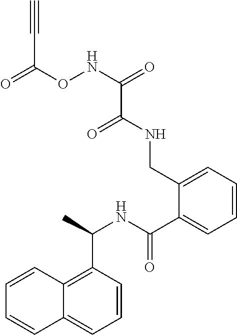 [SMILES: C#CC(=O)ONC(=O)C(=O)NCc1ccccc1C(=O)N[C@H](C)c1cccc2ccccc12] |
| 65a | 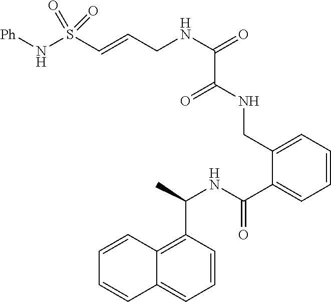 [SMILES: C[C@@H](NC(=O)c1ccccc1CNC(=O)C(=O)NC/C=C/S(=O)(=O)Nc1ccccc1)c1cccc2ccccc12] |
| 66a | 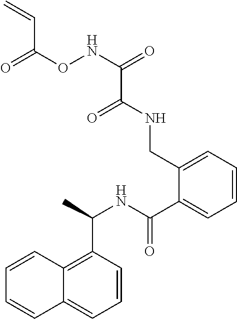 [SMILES: C=CC(=O)ONC(=O)C(=O)NCc1ccccc1C(=O)N[C@H](C)c1cccc2ccccc12] |
| 67a | 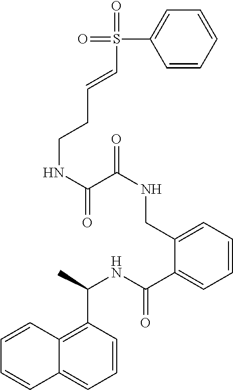 [SMILES: C[C@@H](NC(=O)c1ccccc1CNC(=O)C(=O)NCC/C=C/S(=O)(=O)c1ccccc1)c1cccc2ccccc12] |
| 68a | 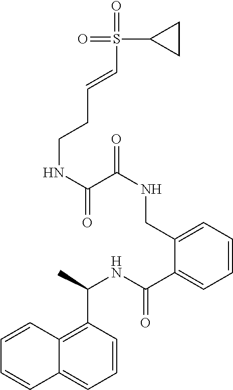 [SMILES: C[C@@H](NC(=O)c1ccccc1CNC(=O)C(=O)NCC/C=C/S(=O)(=O)C1CC1)c1cccc2ccccc12] |
| 69a | 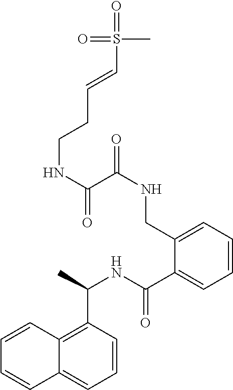 [SMILES: C[C@@H](NC(=O)c1ccccc1CNC(=O)C(=O)NCC/C=C/S(C)(=O)=O)c1cccc2ccccc12] |
| 70a | 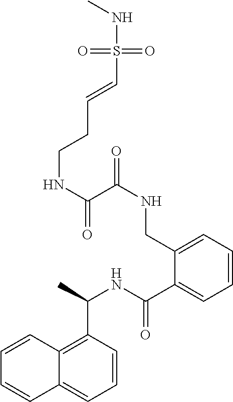 [SMILES: CNS(=O)(=O)/C=C/CCNC(=O)C(=O)NCc1ccccc1C(=O)N[C@H](C)c1cccc2ccccc12] |
| 71a | 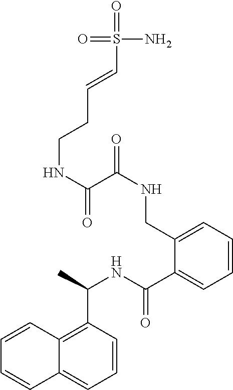 [SMILES: C[C@@H](NC(=O)c1ccccc1CNC(=O)C(=O)NCC/C=C/S(N)(=O)=O)c1cccc2ccccc12] |
| 72a | 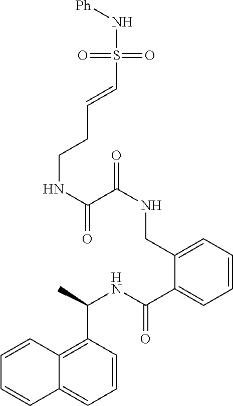 [SMILES: C[C@@H](NC(=O)c1ccccc1CNC(=O)C(=O)NCC/C=C/S(=O)(=O)Nc1ccccc1)c1cccc2ccccc12] |
| 73a | 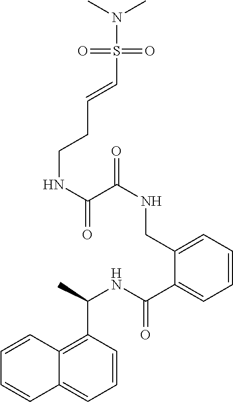 [SMILES: C[C@@H](NC(=O)c1ccccc1CNC(=O)C(=O)NCC/C=C/S(=O)(=O)N(C)C)c1cccc2ccccc12] |
| 74a | 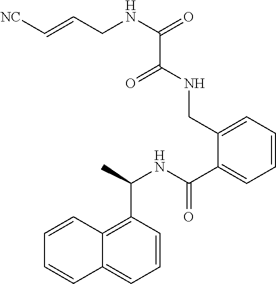 [SMILES: C[C@@H](NC(=O)c1ccccc1CNC(=O)C(=O)NC/C=C/C#N)c1cccc2ccccc12] |
| 75a | 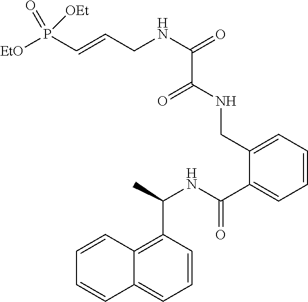 [SMILES: CCOP(=O)(/C=C/CNC(=O)C(=O)NCc1ccccc1C(=O)N[C@H](C)c1cccc2ccccc12)OCC] |
| 76a | 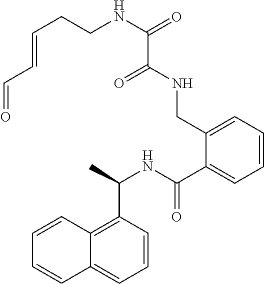 [SMILES: C[C@@H](NC(=O)c1ccccc1CNC(=O)C(=O)NCC/C=C/C=O)c1cccc2ccccc12] |
| 77a | 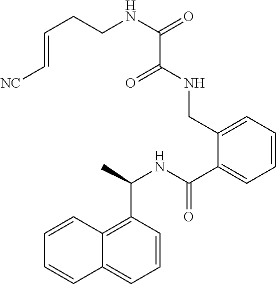 [SMILES: C[C@@H](NC(=O)c1ccccc1CNC(=O)C(=O)NCC/C=C/C#N)c1cccc2ccccc12] |
| 78a | 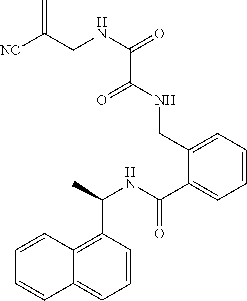 [SMILES: C=C(C#N)CNC(=O)C(=O)NCc1ccccc1C(=O)N[C@H](C)c1cccc2ccccc12] |
| 79a | 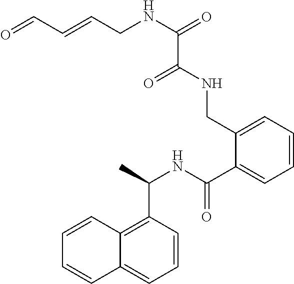 [SMILES: C[C@@H](NC(=O)c1ccccc1CNC(=O)C(=O)NC/C=C/C=O)c1cccc2ccccc12] |
| 80a | 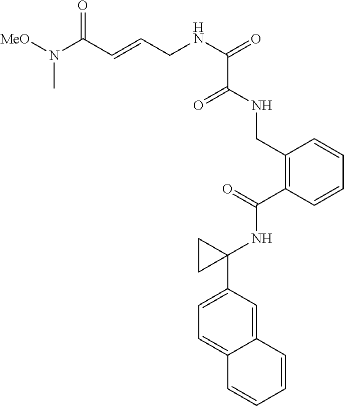 [SMILES: CON(C)C(=O)/C=C/CNC(=O)C(=O)NCc1ccccc1C(=O)NC1(c2ccc3ccccc3c2)CC1] |
| 81a | 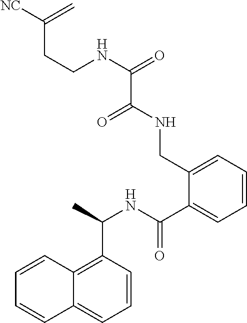 [SMILES: C=C(C#N)CCNC(=O)C(=O)NCc1ccccc1C(=O)N[C@H](C)c1cccc2ccccc12] |
| 82a | 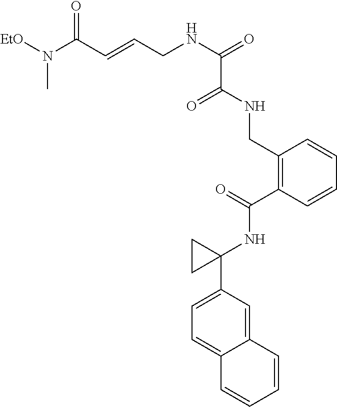 [SMILES: CCON(C)C(=O)/C=C/CNC(=O)C(=O)NCc1ccccc1C(=O)NC1(c2ccc3ccccc3c2)CC1] |
| 83a | 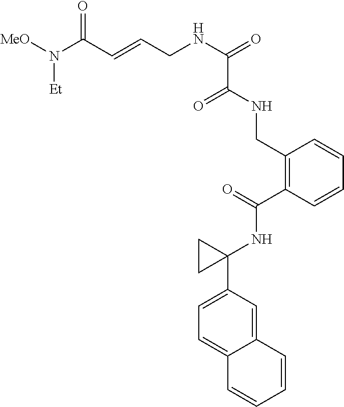 [SMILES: CCN(OC)C(=O)/C=C/CNC(=O)C(=O)NCc1ccccc1C(=O)NC1(c2ccc3ccccc3c2)CC1] |
| 84a | 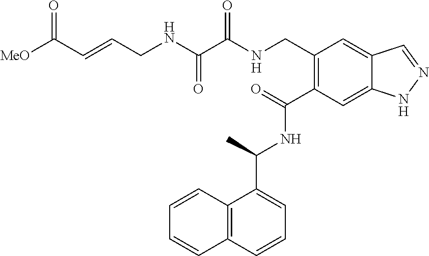 [SMILES: COC(=O)/C=C/CNC(=O)C(=O)NCc1cc2cn[nH]c2cc1C(=O)N[C@H](C)c1cccc2ccccc12] |
| 85a | 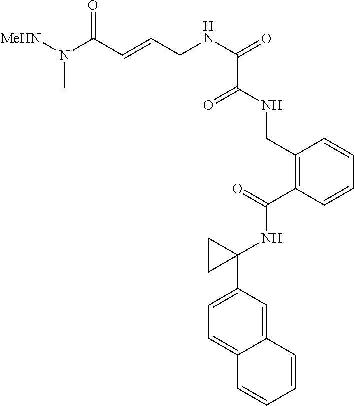 [SMILES: CNN(C)C(=O)/C=C/CNC(=O)C(=O)NCc1ccccc1C(=O)NC1(c2ccc3ccccc3c2)CC1] |
| 86a | 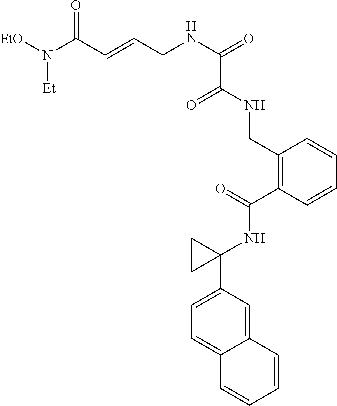 [SMILES: CCON(CC)C(=O)/C=C/CNC(=O)C(=O)NCc1ccccc1C(=O)NC1(c2ccc3ccccc3c2)CC1] |
| 87a | 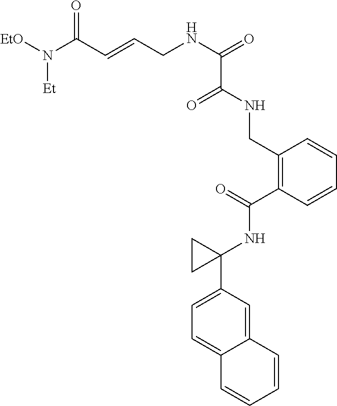 [SMILES: CCON(CC)C(=O)/C=C/CNC(=O)C(=O)NCc1ccccc1C(=O)NC1(c2ccc3ccccc3c2)CC1] |
| 88a | 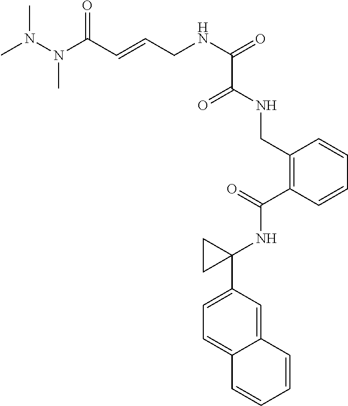 [SMILES: CN(C)N(C)C(=O)/C=C/CNC(=O)C(=O)NCc1ccccc1C(=O)NC1(c2ccc3ccccc3c2)CC1] |
| 89a | 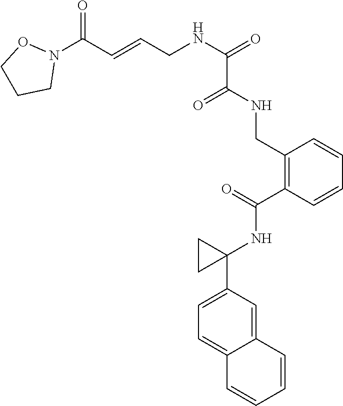 [SMILES: O=C(NC/C=C/C(=O)N1CCCO1)C(=O)NCc1ccccc1C(=O)NC1(c2ccc3ccccc3c2)CC1] |
| 90a | 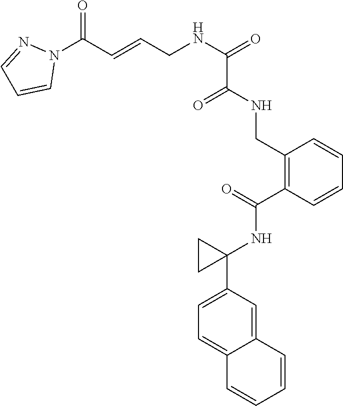 [SMILES: O=C(NC/C=C/C(=O)n1cccn1)C(=O)NCc1ccccc1C(=O)NC1(c2ccc3ccccc3c2)CC1] |
| 91a | 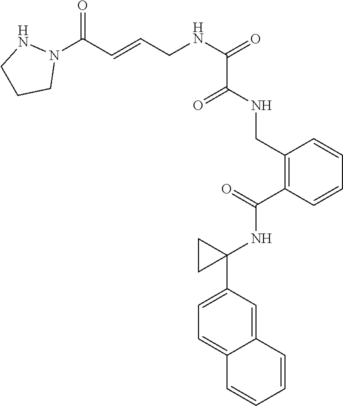 [SMILES: O=C(NC/C=C/C(=O)N1CCCN1)C(=O)NCc1ccccc1C(=O)NC1(c2ccc3ccccc3c2)CC1] |

8. A pharmaceutical composition comprising a compound according to 1 and a pharmaceutically acceptable carrier or excipient.

9. A method of treating or preventing an infection from an RNA-based virus, a coronavirus, a rhinovirus or a norovirus, in a subject in need thereof, the method comprising administering to the subject a therapeutically effective amount of the compound according to claim 1.

10. (canceled)

11. A method of treating or preventing a coronavirus infection in a subject in need thereof, comprising administering to the subject a therapeutically effective amount of a compound according to 1.

12. A method of inhibiting viral Papain-Like protease in a subject, comprising administering to said subject an effective amount of a compound according to claim 1.

13. The method according to claim 12, wherein the subject is a human.

14. A method of treating a respiratory disorder in a subject in need thereof, comprising administering to the subject an effective amount of a compound according to claim 1.

15. The method according to claim 14 wherein the respiratory disorder is acute asthma, lung disease secondary to environmental exposures, an acute lung infection, or a chronic lung infection.

16. The method according to claim 14, wherein the compound or pharmaceutical composition is administered orally, subcutaneously, intravenously or by inhalation.
